(19) United States
(12) Patent Application Publication
(10) Pub. No.: US 2018/0114928 A1
(43) Pub. Date: Apr. 26, 2018

(54) ORGANOMETALLIC COMPOUND, ORGANIC LIGHT-EMITTING DEVICE INCLUDING THE ORGANOMETALLIC COMPOUND, AND DIAGNOSTIC COMPOSITION INCLUDING THE ORGANOMETALLIC COMPOUND

(71) Applicant: SAMSUNG ELECTRONICS CO., LTD., Suwon-si (KR)

(72) Inventors: Jungin LEE, Hwaseong-si (KR); Kyuyoung HWANG, Anyang-si (KR); Soyeon KIM, Seoul (KR); Hyeonho CHOI, Seoul (KR); Seungyeon KWAK, Suwon-si (KR); Yoonhyun KWAK, Seoul (KR); Sunghun LEE, Hwaseong-si (KR); Byoungki CHOI, Hwaseong-si (KR)

(21) Appl. No.: 15/730,159

(22) Filed: Oct. 11, 2017

(30) Foreign Application Priority Data
Oct. 26, 2016 (KR) 10-2016-0140443

(51) Int. Cl. H01L 51/00; H01L 51/50; C07D 401/14; C09K 11/87

(52) U.S. Cl. CPC H01L 51/0087; H01L 51/50; C09K 2211/185; C09K 11/87; C07D 401/14

(57) ABSTRACT

An organometallic compound, represented by Formula 1:

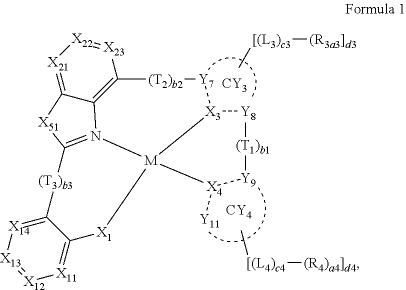

wherein, in Formula 1, groups and variables are the same as described in the specification.

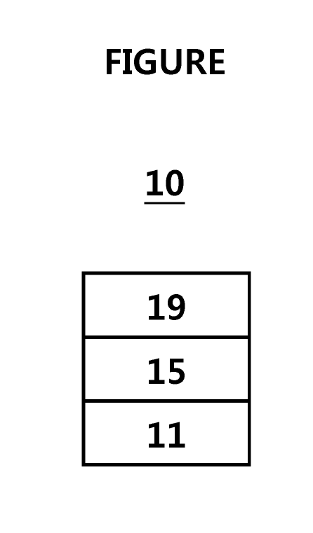

### CROSS-REFERENCE TO RELATED APPLICATION

This application claims priority to Korean Patent Application No. 10-2016-0140443, filed on Oct. 26, 2016, in the Korean Intellectual Property Office, and all the benefits accruing therefrom under 35 U.S.C. § 119, the content of which is incorporated herein in its entirety by reference.

### BACKGROUND

### 1. Field

One or more embodiments relate to an organometallic compound, an organic light-emitting device including the organometallic compound, and a diagnostic composition including the organometallic compound.

### 2. Description of the Related Art

Organic light-emitting devices (OLEDs) are self-emission devices, which have superior characteristics in terms of a viewing angle, a response time, and a brightness, a driving voltage, and a response speed, and which produce full-color images.

In an example, an organic light-emitting device includes an anode, a cathode, and an organic layer disposed between the anode and the cathode, wherein the organic layer includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transit from an excited state to a ground state, thereby generating light.

Meanwhile, luminescent compounds may be used to monitor, sense, or detect a biological material such as a cell protein. Examples of such luminescent compounds include a phosphorescent luminescent compound.

Various types of organic light emitting devices are known. However, there still remains a need in OLEDs having low driving voltage, high efficiency, high brightness, and long lifespan.

### SUMMARY

One or more embodiments include a novel organometallic compound, an organic light-emitting device including the organometallic compound, and a diagnostic composition including the organometallic compound.

Additional aspects will be set forth in part in the description which follows and, in part, will be apparent from the description, or may be learned by practice of the presented embodiments.

According to one or more embodiments, an organometallic compound is represented by Formula 1:

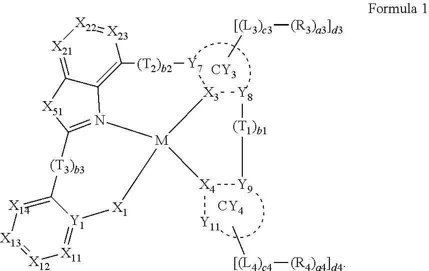

In Formula 1,
M may be beryllium (Be), magnesium (Mg), aluminum (Al), calcium (Ca), titanium (Ti), manganese (Mn), cobalt (Co), copper (Cu), zinc (Zn), gallium (Ga), germanium (Ge), zirconium (Zr), ruthenium (Ru), rhodium (Rh), palladium (Pd), silver (Ag), rhenium (Re), platinum (Pt), or gold (Au),
X₁ may be O or S, and a bond between X₁ and M may be a covalent bond,
X₃ and X₄ may each independently be N or C, a bond between N and M may be a coordinate bond, and one bond selected from a bond between X₃ and M and a bond between X₄ and M may be a covalent bond and the other thereof may be a coordinate bond,
Y₁, Y₇, Y₈, and Y₉ may each independently be C or N,
Y₁₁ may be C, N, O, or S,
a bond between X₃ and Y₇, a bond between X₃ and Y₈, a bond between X₄ and Y₉, and a bond between X₄ and Y₁₁ may each independently be a single bond or a double bond,
CY₃ and CY₄ may each independently be selected from a C₅-C₃₀ carbocyclic group and a C₁-C₃₀ heterocyclic group,
X₁₁ may be N or C-[(L₁₁)_{c11}-(R₁₁)ₐ₁₁], X₁₂ may be N or C-[(L₁₂)_{c12}-(R₁₂)ₐ₁₂], X₁₃ may be N or C-[(L₁₃)_{c13}-(R₁₃)ₐ₁₃], X₁₄ may be N or C-[(L₁₄)_{c14}-(R₁₄)ₐ₁₄], X₂₁ may be N or C-[(L₂₁)_{c21}-(R₂₁)ₐ₂₁], X₂₂ may be N or C-[(L₂₂)_{c22}-(R₂₂)ₐ₂₂], and X₂₃ may be N or C-[(L₂₃)_{c23}-(R₂₃)ₐ₂₃], provided that at least one of X₁₁ to X₁₄ and X₂₁ to X₂₃ is N,
T₁ to T₃ may each independently be selected from *—O—*′, *—S—*′, *—C(R₅)(R₆)—*′, *—C(R₅)=*′, *═C(R₅)—*′, *—C(R₅)═C(R₆)—*′, *—C(═O)—*′, *—C(═S)—*′, *—C≡C—*′, *—N(R₅)—*′, *—Si(R₅)(R₆)—*′, and *—P(R₅)(R₆)—*,
R₅ and R₆ may optionally be linked via a first linking group to form a substituted or unsubstituted C₅-C₃₀ carbocyclic group or a substituted or unsubstituted C₁-C₃₀ heterocyclic group,
b1 to b3 may each independently be 0, 1, 2, or 3, wherein, when b1 is zero, *-(T₁)_{b1}-*′ may be a single bond, when b1 is two or more, two or more groups T₁ may be identical to or different from each other, when b2 is zero, *-(T₂)_{b2}-*′ may be a single bond, when b2 is two or more, two or more groups T₂ may be identical to or different from each other, when b3 is zero, *-(T₃)_{b3}-*′ may be a single bond, and when b3 is two or more, two or more groups T₃ may be identical to or different from each other,
X₅₁ may be selected from O, S, N-[(L₇)_{c7}-(R₇)ₐ₇], C(R₇)(R₈), Si(R₇)(R₈), and C(═O),
R₇ and R₈ may optionally be linked via a second linking group to form a substituted or unsubstituted C₅-C₃₀ carbocyclic group or a substituted or unsubstituted C₁-C₃₀ heterocyclic group,
L₃, L₄, L₇, L₁₁ to L₁₄, and L₂₁ to L₂₃ may each independently be selected from a single bond, a substituted or unsubstituted C₅-C₃₀ carbocyclic group, and a substituted or unsubstituted C₁-C₃₀ heterocyclic group,
c3, c4, c7, cl11 to c14, and c21 to c23 may each independently be an integer from 1 to 5,
R₃ to R₈, R₁₁ to R₁₄, and R₂₁ to R₂₃ may each independently be selected from hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),
a3, a4, a7, a11 to a14, a21 to a23, d3, and d4 may each independently be 0, 1, 2, 3, 4, or 5,
two of *-(L₁₁)_{c11}-(R₁₁)ₐ₁₁, *-(L₁₂)_{c12}-(R₁₂)ₐ₁₂, *-(L₁₃)_{c13}-(R₁₃)ₐ₁₃, and *-(L₁₄)_{c14}-(R₁₄)ₐ₁₄ may optionally be linked to form a substituted or unsubstituted C₅-C₃₀ carbocyclic group or a substituted or unsubstituted C₁-C₃₀ heterocyclic group,
two of *-(L₂₁)_{c21}-(R₂₁)ₐ₂₁, *-(L₂₂)_{c22}-(R₂₂)ₐ₂₂, and *-(L₂₃)_{c23}-(R₂₃)ₐ₂₃ may optionally be linked to form a substituted or unsubstituted C₅-C₃₀ carbocyclic group or a substituted or unsubstituted C₁-C₃₀ heterocyclic group,
two of groups *-(L₃)_{c3}-(R₃)ₐ₃ in the number of d3 may optionally be linked to form a substituted or unsubstituted C₅-C₃₀ carbocyclic group or a substituted or unsubstituted C₁-C₃₀ heterocyclic group,
two of groups *-(L₄)_{c4}-(R₄)ₐ₄ in the number of d4 may optionally be linked to form a substituted or unsubstituted C₅-C₃₀ carbocyclic group or a substituted or unsubstituted C₁-C₃₀ heterocyclic group,
* and *′ each indicate a binding site to a neighboring atom,
at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₂-C₆₀ heteroaryloxy group, the substituted C₂-C₆₀ heteroarylthio group, the substituted C₃-C₆₀ heteroarylalkyl group, the substituted monovalent non-aromatic condensed polycyclic group, and substituted monovalent non-aromatic condensed heteropolycyclic group may be selected from:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and
—N(Q₃₁)(Q₃₂), —Si(Q₃₃)(Q₃₄)(Q₃₅), —B(Q₃₆)(Q₃₇), and —P(═O)(Q₃₈)(Q₃₉), and
Q₁ to Q₉, Q₁₁ to Q₁₉, Q₂₁ to Q₂₉, and Q₃₁ to Q₃₉ may each independently be selected from hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

According to one or more embodiments, an organic light-emitting device includes:
a first electrode;
a second electrode; and
an organic layer disposed between the first electrode and the second electrode,
wherein the organic layer includes an emission layer and at least one organometallic compound.

The organometallic compounds may act as a dopant in the emission layer.

According to one or more embodiments, a diagnostic composition includes at least one organometallic compound represented by Formula 1.

### BRIEF DESCRIPTION OF THE DRAWING

These and/or other aspects will become apparent and more readily appreciated from the following description of the embodiments, taken in conjunction with the FIGURE which is a schematic view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present disclosure. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10%, 5% of the stated value.

An organometallic compound according to an embodiment may be represented by Formula 1:

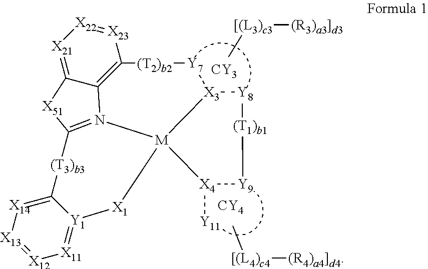

M in Formula 1 may be beryllium (Be), magnesium (Mg), aluminum (Al), calcium (Ca), titanium (Ti), manganese (Mn), cobalt (Co), copper (Cu), zinc (Zn), gallium (Ga), germanium (Ge), zirconium (Zr), ruthenium (Ru), rhodium (Rh), palladium (Pd), silver (Ag), rhenium (Re), platinum (Pt), or gold (Au).

In an embodiment, M in Formula 1 may be platinum, but embodiments of the present disclosure are not limited thereto.

The organometallic compound represented by Formula 1 may be a neutral compound which does not consist of an ion pair of an anion and a cation.

In Formula 1, X₁ may be O or S, and a bond between X₁ and M may be a covalent bond.

In Formula 1, X₃ and X₄ may each independently be N or C, a bond between N and M may be a coordinate bond, and one of a bond selected from a bond between X₃ and M and a bond between X₄ and M may be a covalent bond, and the other may be a coordinate bond.

For example, X₁ in Formula 1 may be 0, but embodiments of the present disclosure are not limited thereto.

In Formula 1, Y₁, Y₇, Y₈, and Y₉ may each independently be C or N, Y₁₁ may be C, N, O, or S, and a bond between X₃ and Y₇, a bond between X₃ and Y₈, a bond between X₄ and Y₉, and a bond between X₄ and Y₁₁ may each independently be a single bond or a double bond.

For example, Y₁, Y₇, Y₈, and Y₉ in Formula 1 may each be C, and Y₁₁ may be N or C.

In an embodiment, in Formula 1, X₃ may be C, X₄ may be N, a bond between X₃ and M may be a covalent bond, a bond between X₄ and M may be a coordinate bond, and Y₁, Y₇, Y₈, and Y₉ may each be C, but embodiments of the present disclosure are not limited thereto.

CY₃ and CY₄ in Formula 1 may each independently be selected from a C₅-C₃₀ carbocyclic group and a C₁-C₃₀ heterocyclic group.

For example, in Formula 1,
CY₃ may be selected from a benzene group, a naphthalene group, an anthracene group, a phenanthrene group, a triphenylene group, a pyrene group, a chrysene group, a cyclopentadiene group, an indene group, a fluorene group, a pyrrole group, an indole group, a carbazole group, a furan group, a benzofuran group, a dibenzofuran group, a thiophene group, a benzothiophene group, a dibenzothiophene group, a pyridine group, a pyrimidine group, and a 1,2,3,4-tetrahydronaphthalene group, and
CY₄ may be selected from a pyridine group, a pyrimidine group, a pyrazine group, a pyridazine group, a triazine group, a quinoline group, an isoquinoline group, a quinoxaline group, a quinazoline group, a phenanthroline group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, and a 5,6,7,8-tetrahydroquinoline group.

In an embodiment, in Formula 1,
CY₃ may be a benzene group, a naphthalene group, a fluorene group, a dibenzofuran group, a dibenzothiophene group, or a 1,2,3,4-tetrahydronaphthalene group, and
CY₄ may be a pyridine group, a pyrimidine group, a quinoline group, an isoquinoline group, a quinoxaline group, a quinazoline group, a 5,6,7,8-tetrahydroisoquinoline group, or a 5,6,7,8-tetrahydroquinoline group.

In an embodiment, in Formula 1, CY₃ may be a benzene group or a pyridine group, and CY₄ may be a pyridine group, a quinoline group, an isoquinoline group, a 5,6,7,8-tetrahydroisoquinoline group, or a 5,6,7,8-tetrahydroquinoline group, but embodiments of the present disclosure are not limited thereto.

In Formula 1, X₁₁ may be N or C-[(L₁₁)_{c11}-(R₁₁)ₐ₁₁], X₁₂ may be N or C-[(L₁₂)_{c12}-(R₁₂)ₐ₁₂], X₁₃ may be N or C-[(L₁₃)_{c13}-(R₁₃)ₐ₁₃], X₁₄ may be N or C-[(L₁₄)_{c14}-(R₁₄)ₐ₁₄], X₂₁ may be N or C-[(L₂₁)_{c21}-(R₂₁)ₐ₂₁], X₂₂ may be N or C-[(L₂₂)_{c22}-(R₂₂)ₐ₂₂], X₂₃ may be N or C-[(L₂₃)_{c23}-(R₂₃)ₐ₂₃], provided that at least one of X₁₁ to X₁₄ and X₂₁ to X₂₃ may be N.

For example, in Formula 1, 1) one or two of X₁₁ to X₁₄ may be N and all of X₂₁ to X₂₃ may not be N, 2) all of X₁₁ to X₁₄ may not be N and one or two of X₂₁ to X₂₃ may be N, or 3) one or two of X₁₁ to X₁₄ may be N and one or two of X₂₁ to X₂₃ may be N.

In an embodiment, in Formula 1, 1) one of X₁₁ to X₁₄ may be N and the others thereof may not be N, and all of X₂₁ to X₂₃ may not be N, 2) all of X₁₁ to X₁₄ may not be N, and one of X₂₁ to X₂₃ may be N and the others thereof may not be N, or 3) one of X₁₁ to X₁₄ may be N and the others may not be N, and one of X₂₁ to X₂₃ may be N and the others thereof may not be N.

T₁ to T₃ in Formula 1 may each independently be selected from *—O—*′, *—S—*′, *—C(R₅)(R₆)—*′, *—C(R₅)=*′, *═C(R₅)—*′, *—C(R₅)═C(R₆)—*′, *—C(═O)—*′, *—C(═S)—*′, *—C≡C—*′, *—N(R₅)—*′, *—Si(R₅)(R₆)—*′, and *—P(R₅)(R₆)—*′. R₅ and R₆ are the same as described below. * and *′ each indicate a binding site to a neighboring atom.

R₅ and R₆ may optionally be linked via a first linking group to form a substituted or unsubstituted C₅-C₃₀ carbocyclic group or a substituted or unsubstituted C₁-C₃₀ heterocyclic group.

In an embodiment, T₁ to T₃ in Formula 1 may each independently be selected from *—O—*′, *—S—*′, *—C(R₅)(R₆)—*′, *—N(R₅)—*′, *—Si(R₅)(R₆)—*′, and *—P(R₅)(R₆)—*′.

In one or more embodiments, in Formula 1,
T₁ to T₃ may each independently be selected from *—C(R₅)(R₆)—*′, *—Si(R₅)(R₆)—*′ and *—P(R₅)(R₆)—*′,
R₅ and R₆ may be linked via a first linking group,
the first linking group may be selected from a single bond, *—O—*′, *—S—*′, *—C(R₉)(R₁₀)—*′, *—C(R₉)=*′, *═C(R₉)—*′, *—C(R₉)═C(R₁₀)—*′, *—C(═O)—*′, *—C(═S)—*′, *—C≡C—*′, *—N(R₉)—*′, *—Si(R₉)(R₁₀)—*′, and *—P(R₉)(R₁₀)—*′,
R₉ and R₁₀ are the same as described in connection with R₅, and
* and *′ each indicate a binding site to a neighboring atom, but embodiments of the present disclosure are not limited thereto.

b1, b2, and b3 in Formula 1 respectively indicate the number of T₁, the number of T₂, and the number of T₃, and may each independently be 0, 1, 2, or 3. When b1 is zero, *-(T₁)_{b1}-*′ may be a single bond, when b1 is two or more, two or more groups T₁ may be identical to or different from each other, when b2 is zero, *-(T₂)_{b2}-*′ may be a single bond, when b2 is two or more, two or more groups T₂ may be identical to or different from each other, when b3 is zero, *-(T₃)_{b3}-*′ may be a single bond, and when b3 is two or more, two or more groups T₃ may be identical to or different from each other.

For example, b1 to b3 may each independently be 0 or 1.

The sum of b1, b2, and b3 in Formula 1 may be 0, or may be 1 or more.

In one or more embodiments, in Formula 1,
all of b1, b2, and b3 may be 0;
b1 may be 1 and b2 and b3 may each be 0;
b2 may be 1 and b1 and b3 may each be 0; or
b3 may be 1 and b1 and b2 may each be 0, but embodiments of the present disclosure are not limited thereto.

X₅₁ in Formula 1 may be selected from O, S, N(R₇), C(R₇)(R₈), Si(R₇)(R₈), and C(═O). R₇ and R₈ are the same as described herein.

R₇ and R₈ may optionally be linked via a second linking group to form a substituted or unsubstituted C₅-C₃₀ carbocyclic group or a substituted or unsubstituted C₁-C₃₀ heterocyclic group.

The first linking group and the second linking group may each independently be selected from a single bond, *—O—*′, *—S—*′, *—C(R₉)(R₁₀)—*′, *—C(R₉)=*′, *═C(R₉)—*′, *—C(R₉)═C(R₁₀)—*′, *—C(═O)—*′, *—C(═S)—*′, *—C≡C—*′, *—N(R₉)—*′, *—Si(R₉)(R₁₀)—*′, and *—P(R₉)(R₁₀)—*′, and R₉ and R₁₀ are the same as described herein.

In an embodiment, X₅₁ in Formula 1 may be selected from O, S, N-[(L₇)_{c7}-(R₇)ₐ₇], C(R₇)(R₈), Si(R₇)(R₈), and C(═O), but embodiments of the present disclosure are not limited thereto.

For example, X₅₁ in Formula 1 may be O, S, or N-[(L₇)_{c7}-(R₇)ₐ₇], but embodiments of the present disclosure are not limited thereto.

R₇ and R₈ may optionally be linked via a second linking group to form a substituted or unsubstituted C₅-C₃₀ carbocyclic group or a substituted or unsubstituted C₁-C₃₀ heterocyclic group.

L₃, L₄, L₇, L₁₁ to L₁₄, and L₂₁ to L₂₃ may each independently be selected from a single bond, a substituted or unsubstituted C₅-C₃₀ carbocyclic group, and a substituted or unsubstituted C₁-C₃₀ heterocyclic group.

For example, L₃, L₄, L₇, L₁₁ to L₁₄, and L₂₁ to L₂₃ may each independently be selected from:
a single bond, a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, and a benzothiadiazole group; and
a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, and a benzothiadiazole group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a pyridinyl group, a pyrimidinyl group, a triazinyl group, a fluorenyl group, a dimethylfluorenyl group, a diphenylfluorenyl group, a carbazolyl group, a phenylcarbazolyl group, a dibenzofuranyl group, a dibenzothiophenyl group, a dibenzosilolyl group, a dimethyldibenzosilolyl group, a diphenyldibenzosilolyl group, —N(Q₃₁)(Q₃₂), —Si(Q₃₃)(Q₃₄)(Q₃₅), —B(Q₃₆)(Q₃₇), and —P(═O)(Q₃₈)(Q₃₉), and
Q₃₁ to Q₃₉ may each independently be selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group; and
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group, each substituted with at least one selected from deuterium, a C₁-C₁₀ alkyl group, and a phenyl group,
but embodiments of the present disclosure are not limited thereto.

c3, c4, c7, c11, c12, c13, c14, c21, c22, and c23 respectively indicate the number of groups L₃, the number of groups L₄, the number of groups L₇, the number of groups L₁₁, the number of groups L₁₂, the number of groups L₁₃, the number of groups L₁₄, the number of groups L₂₁, the number of groups L₂₂, and the number of groups L₂₃, may each independently be an integer from 1 to 5, or may each independently be 1 or 2.

R₃ to R₈, R₁₁ to R₁₄, and R₂₁ to R₂₃ may each independently be selected from hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉). Q₁ to Q₀₉ are each independently the same as described herein.

For example, R₃ to R₈, R₁₁ to R₁₄, and R₂₁ to R₂₃ may each independently be selected from:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, and a C₁-C₂₀ alkoxy group;
a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and
—N(Q₁)(Q₂), —Si(Q₃)(Q₄)(Q₅), —B(Q₆)(Q₇), and —P(═O)(Q₈)(Q₉), and
Q₁ to Q₉ may each independently be selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

In an embodiment, R₃ to R₈, R₁₁ to R₁₄, and R₂₁ to R₂₃ may each independently be selected from:
hydrogen, deuterium, —F, a cyano group, a nitro group, —SF₅, a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group;
a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group, each substituted with at least one selected from deuterium, —F, —CD₃, —CD₂H, —CDH₂, —CF₃, —CF₂H, —CFH₂, a cyano group, a nitro group, a C₁-C₁₀ alkyl group, a C₁-C₁₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group; and
—N(Q₁)(Q₂), —Si(Q₃)(Q₄)(Q₅), —B(Q₆)(Q₇), and —P(═O)(Q₈)(Q₉), and
Q₁ to Q₉ are each independently the same as described herein.

In an embodiment, R₃ to R₈, R₁₁ to R₁₄, and R₂₁ to R₂₃ may each independently be selected from hydrogen, deuterium, —F, a cyano group, a nitro group, —SF₅, —CH₃, —CD₃, —CD₂H, —CDH₂, —CF₃, —CF₂H, —CFH₂, groups represented by Formulae 9-1 to 9-19, groups represented by Formulae 10-1 to 10-139, and —Si(Q₃)(Q₄)(Q₅), but embodiments of the present disclosure are not limited thereto:

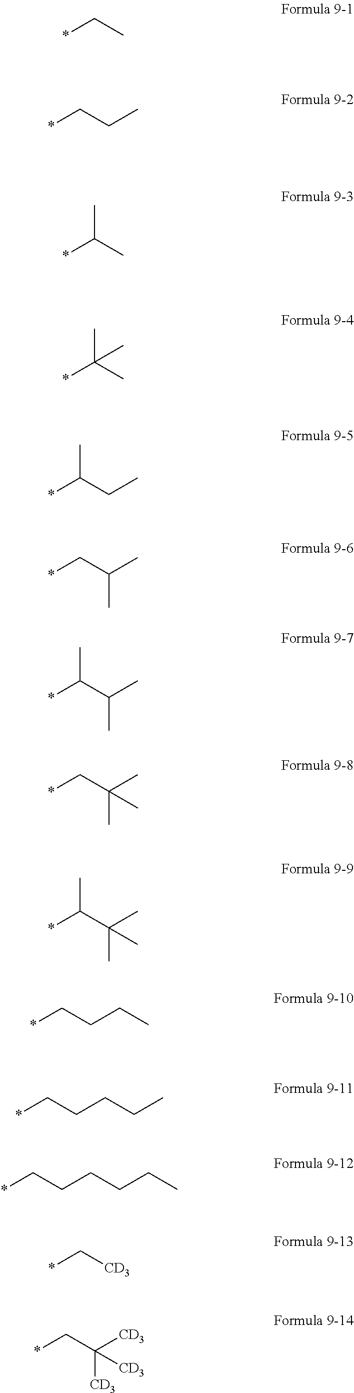

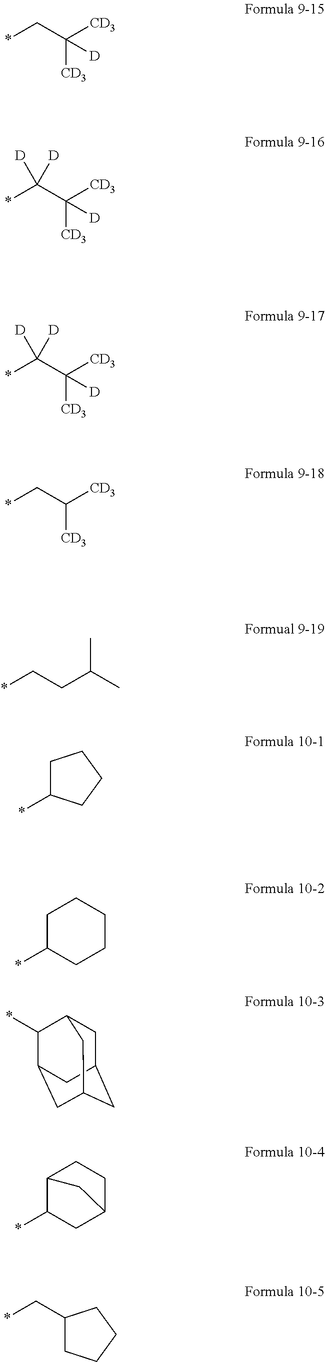

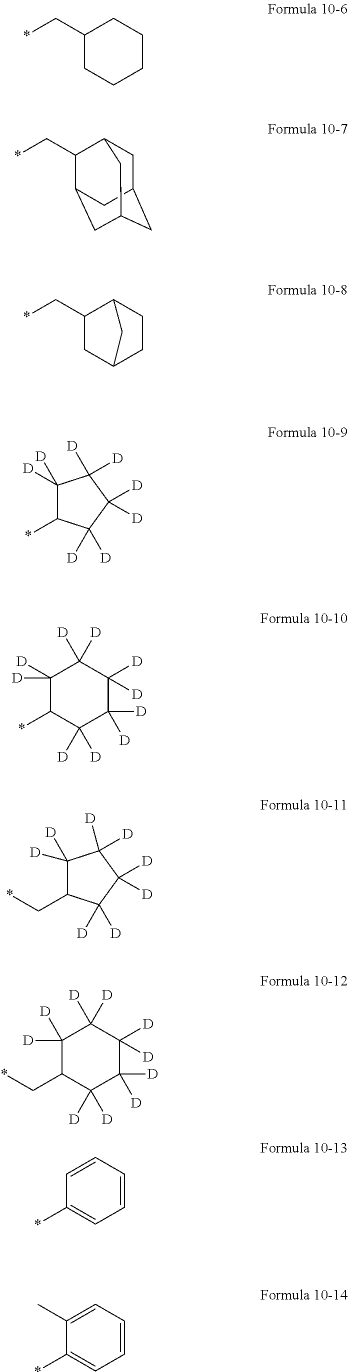

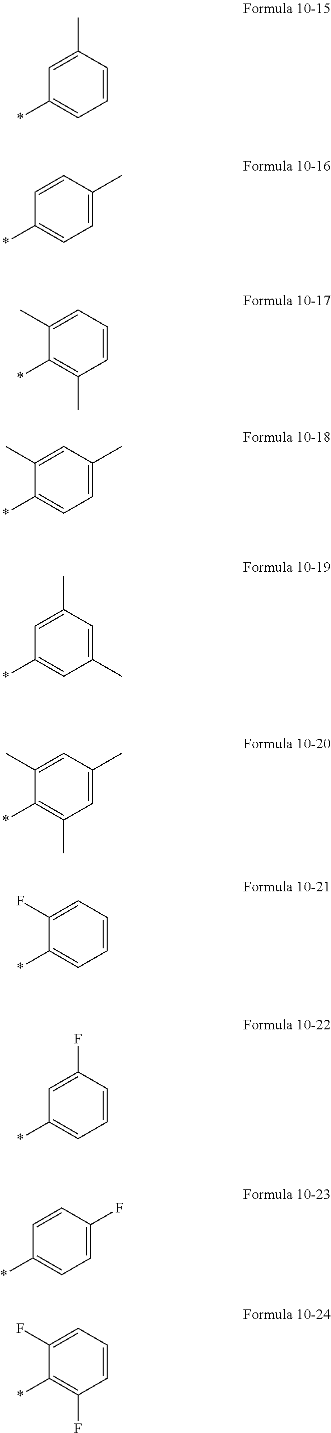

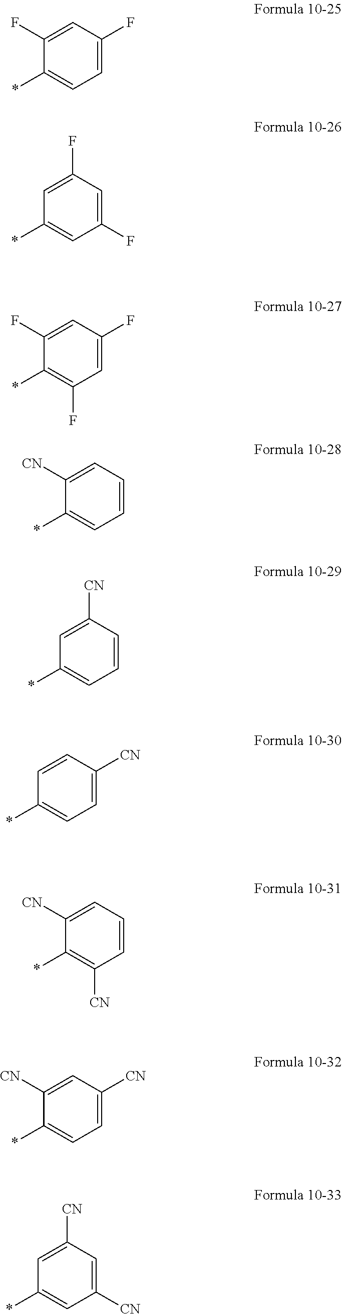

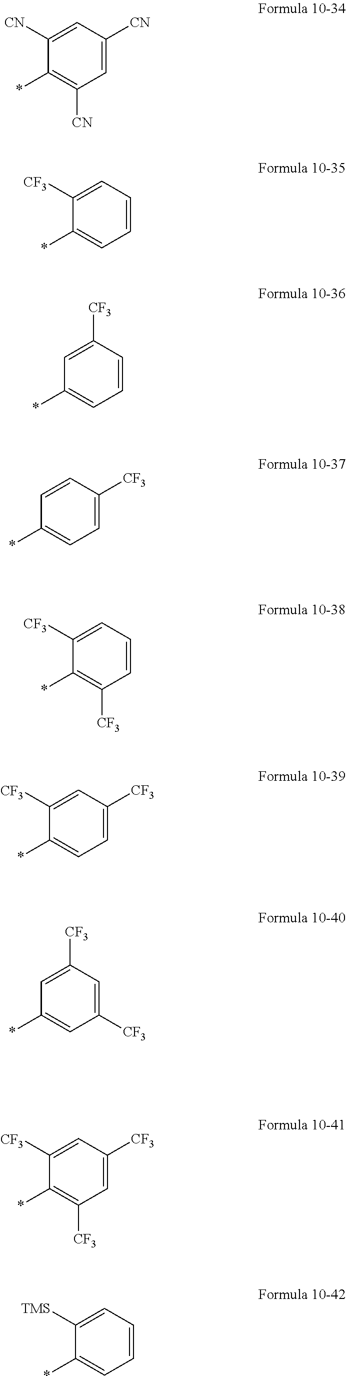

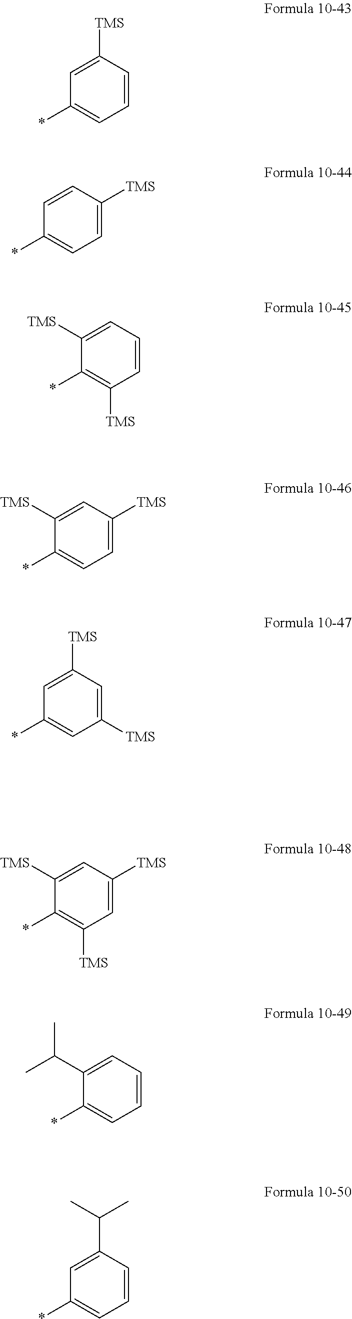

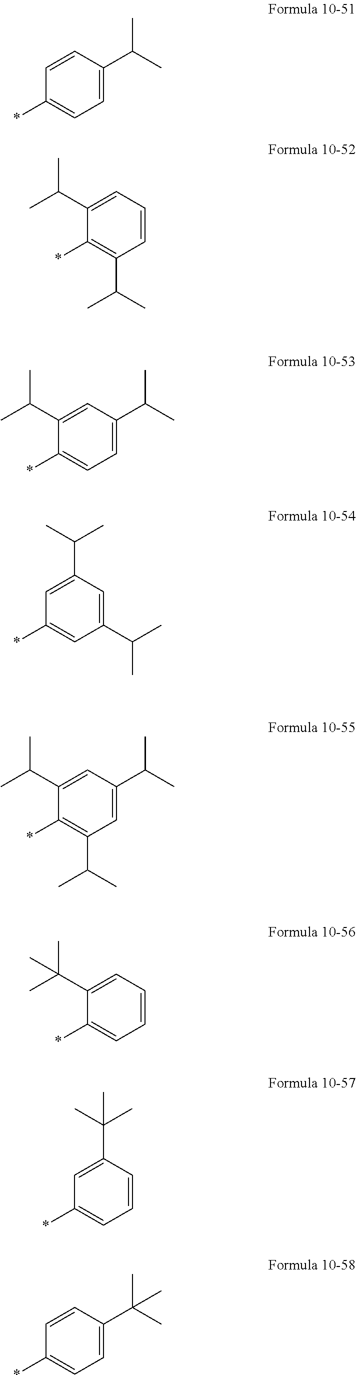

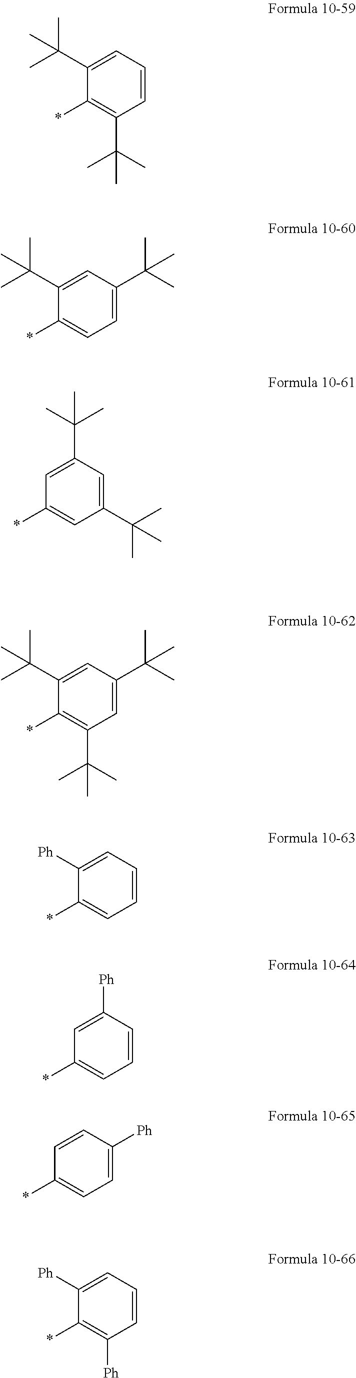

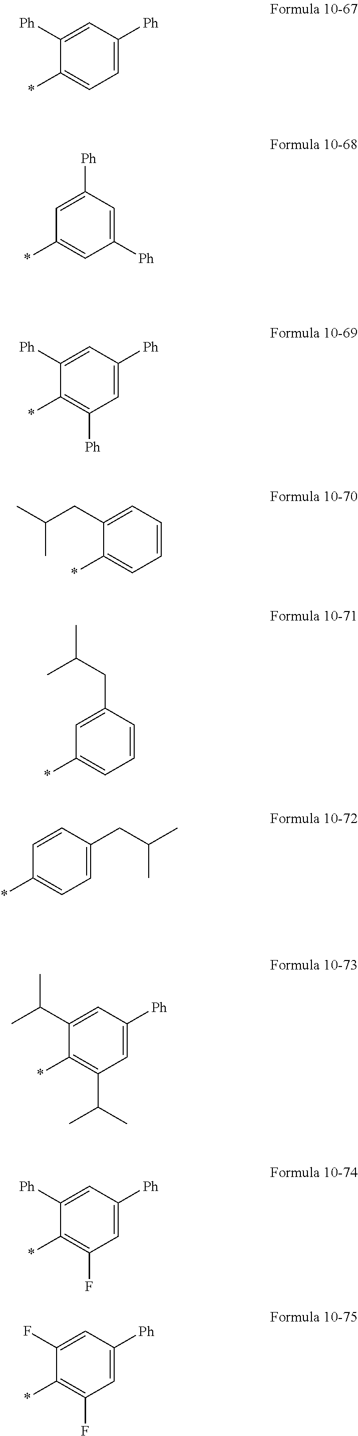

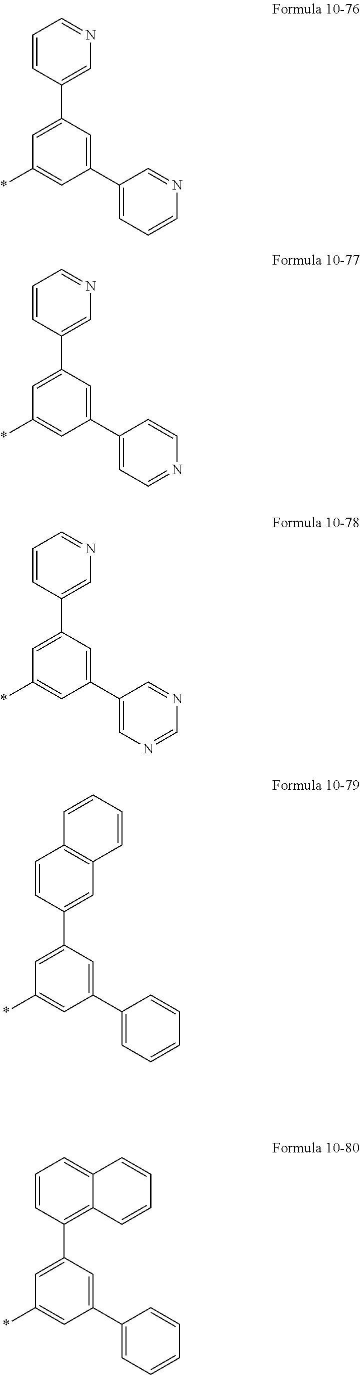 [SMILES: *c1cc(-c2cccnc2)cc(-c2cccnc2)c1 ; *c1cc(-c2ccncc2)cc(-c2cccnc2)c1 ; *c1cc(-c2cccnc2)cc(-c2cncnc2)c1 ; *c1cc(-c2ccccc2)cc(-c2ccc3ccccc3c2)c1 ; *c1cc(-c2ccccc2)cc(-c2cccc3ccccc23)c1]

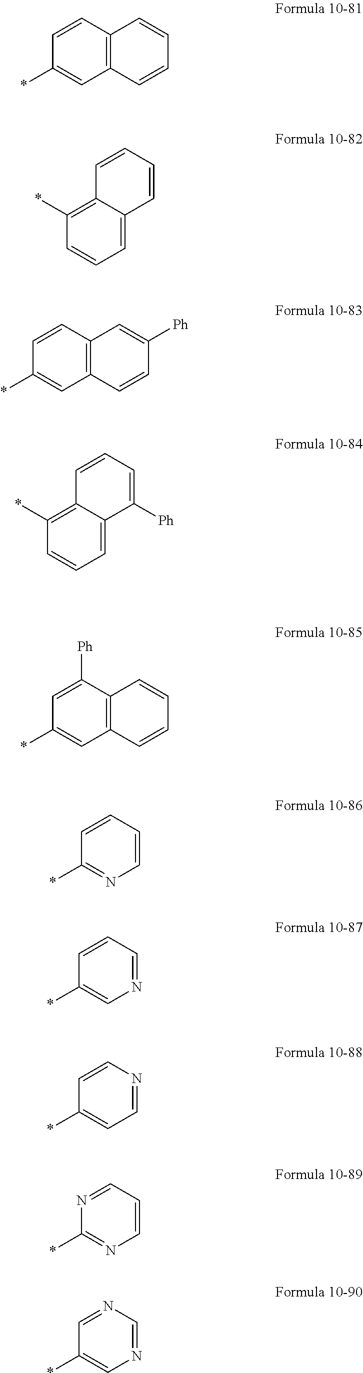

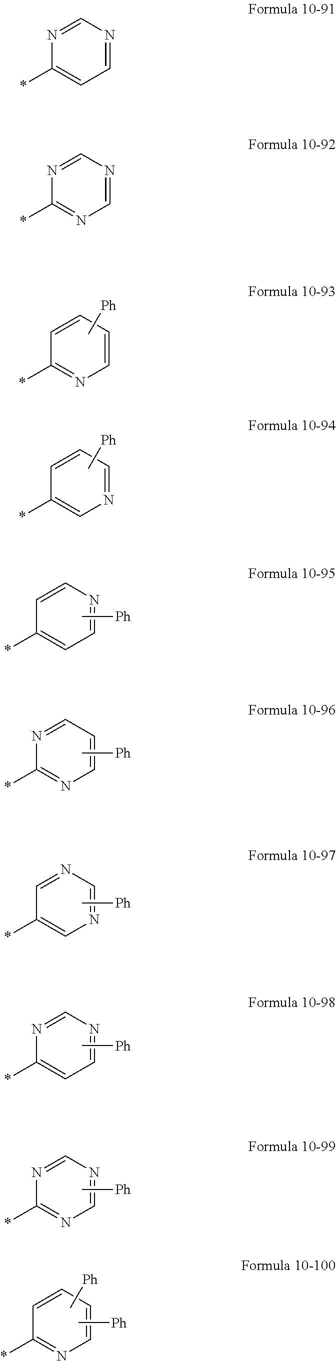

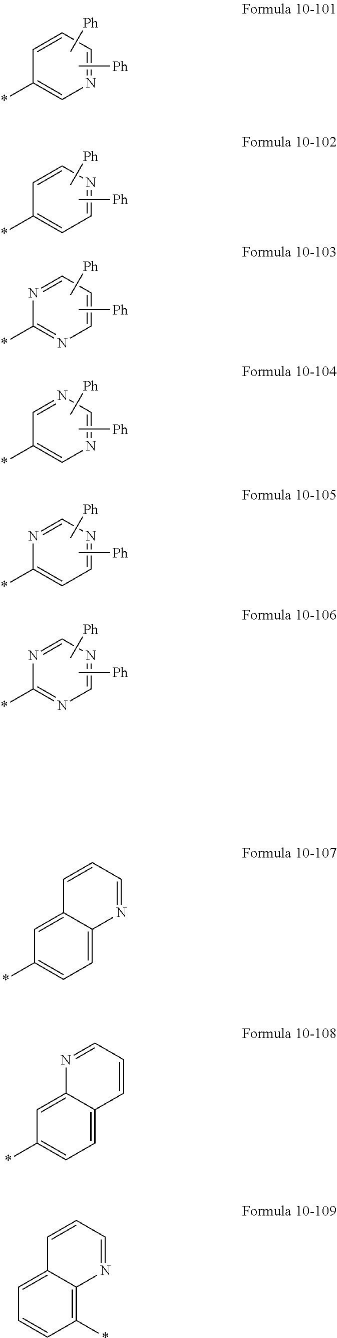

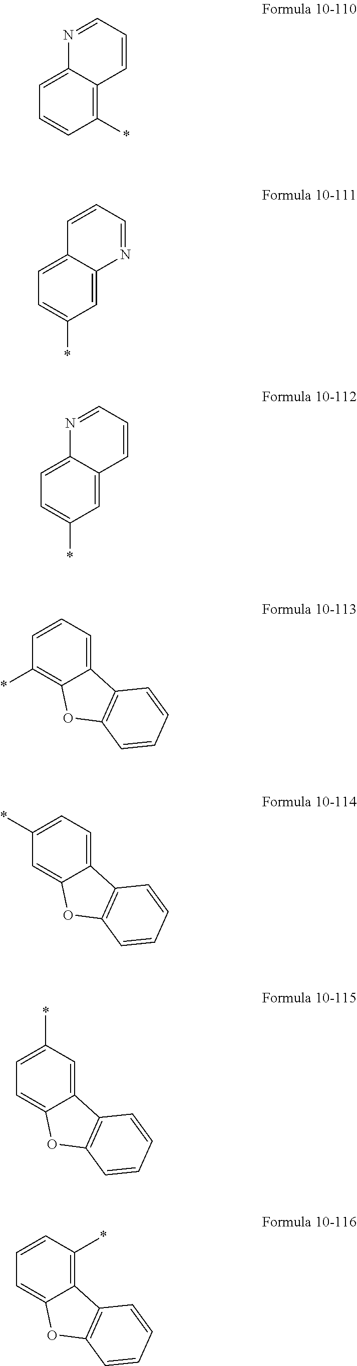

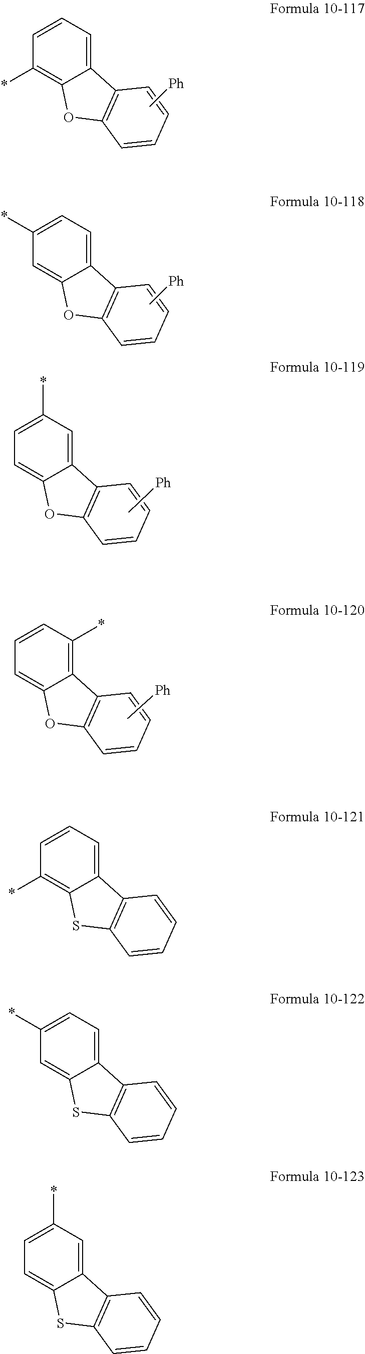

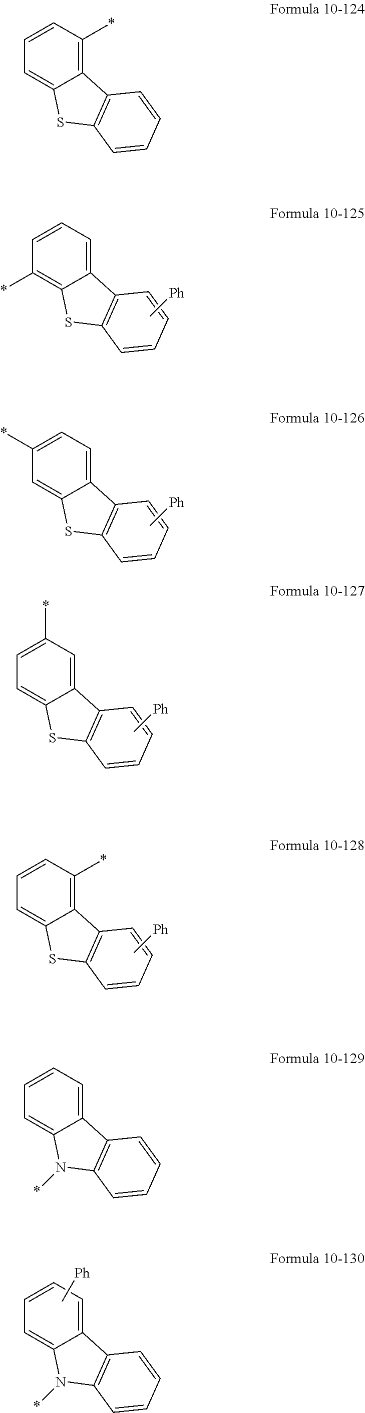

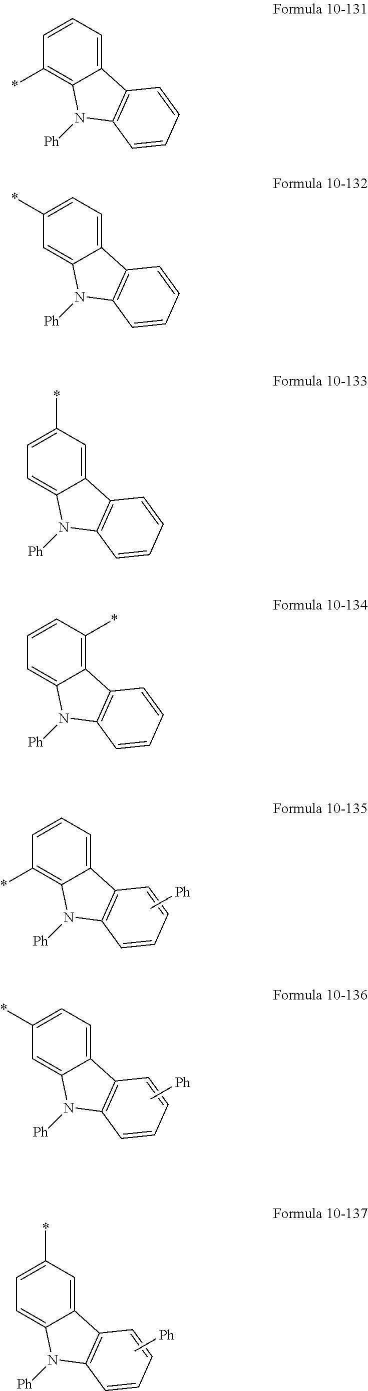

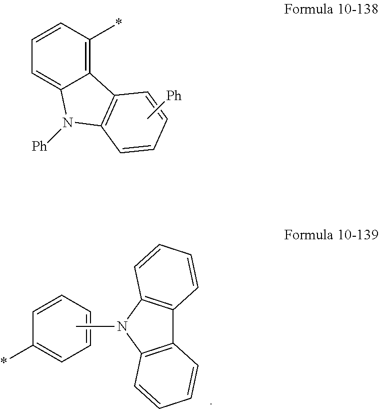

Q₃ to Q₉ may each independently be selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group, each substituted with at least one selected from an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group; and
deuterium, a C₁-C₁₀ alkyl group, and a phenyl group,
wherein, in Formulae 9-1 to 9-19 and 10-1 to 10-139,
* indicates a binding site to a neighboring atom,
Ph indicates a phenyl group, and
TMS indicates a trimethylsilyl group.

a3, a4, a7, a11 to a14, a21 to a23, d3, and d4 in Formula 1 may each independently be 0, 1, 2, 3, 4, or 5, for example, 0, 1, or 2.

In Formula 1, two of *-(L₁₁)_{c11}-(R₁₁)ₐ₁₁, *-(L₁₂)_{c12}-(R₁₂)ₐ₁₂, *-(L₁₃)_{c13}-(R₁₃)ₐ₁₃, and *-(L₁₄)_{c14}-(R₁₄)ₐ₁₄ may optionally be linked to form a substituted or unsubstituted C₅-C₃₀ carbocyclic group or a substituted or unsubstituted C₁-C₃₀ heterocyclic group, two of *-(L₂₁)_{c21}-(R₂₁)ₐ₂₁, *-(L₂₂)_{c22}-(R₂₂)ₐ₂₂, and *-(L₂₃)_{c23}-(R₂₃)ₐ₂₃ may optionally be linked to form a substituted or unsubstituted C₅-C₃₀ carbocyclic group or a substituted or unsubstituted C₁-C₃₀ heterocyclic group, two of groups *-(L₃)_{c3}-(R₃)ₐ₃ in the number of d3 may optionally be linked to form a substituted or unsubstituted C₅-C₃₀ carbocyclic group or a substituted or unsubstituted C₁-C₃₀ heterocyclic group, and two groups *-(L₄)_{c4}-(R₄)ₐ₄ in the number of d4 may optionally be linked to form a substituted or unsubstituted C₅-C₃₀ carbocyclic group or a substituted or unsubstituted C₁-C₃₀ heterocyclic group.

For example, in Formula 1, the substituted or unsubstituted C₅-C₃₀ carbocyclic group or the substituted or unsubstituted C₁-C₃₀ heterocyclic group, which may be formed by optionally linking two of *-(L₁₁)_{c11}-(R₁₁)ₐ₁₁, *-(L₁₂)_{c12}-(R₁₂)ₐ₁₂, *-(L₁₃)_{c13}-(R₁₃)ₐ₁₃, and *-(L₁₄)_{c14}-(R₁₄)ₐ₁₄, the substituted or unsubstituted C₅-C₃₀ carbocyclic group or the substituted or unsubstituted C₁-C₃₀ heterocyclic group, which may be formed by optionally linking two of *-(L₂₁)_{c21}-(R₂₁)ₐ₂₁, *-(L₂₂)_{c22}-(R₂₂)ₐ₂₂, and *-(L₂₃)_{c23}-(R₂₃)ₐ₂₃, the substituted or unsubstituted C₅-C₃₀ carbocyclic group or the substituted or unsubstituted C₁-C₃₀ heterocyclic group, which may be formed by optionally linking two of groups *-(L₃)_{c3}-(R₃)ₐ₃ in the number of d3, and the substituted or unsubstituted C₅-C₃₀ carbocyclic group or the substituted or unsubstituted C₁-C₃₀ heterocyclic group, which may be formed by optionally linking two of groups *-(L₄)_{c4}-(R₄)ₐ₄ in the number of d4, may be selected from:
a pentadiene group, a cyclohexane group, an adamantane group, a bicyclo[2.2.1]heptane group, a benzene group, a pyridine group, a pyrimidine group, a pyrazine group, a pyridazine group, a naphthalene group, an anthracene group, a tetracene group, a phenanthrene group, a dihydronaphthalene group, a phenalene group, a benzothiophene group, a benzofuran group, an indene group, and an indole group; and
a pentadiene group, a cyclohexane group, an adamantane group, a bicyclo[2.2.1]heptane group, a benzene group, a pyridine group, a pyrimidine group, a pyrazine group, a pyridazine group, a naphthalene group, an anthracene group, a tetracene group, a phenanthrene group, a dihydronaphthalene group, a phenalene group, a benzothiophene group, a benzofuran group, an indene group, and an indole group, each substituted with at least one R₁ₐ,
but embodiments of the present disclosure are not limited thereto.

R₁ₐ is the same as described in connection with R₃.

In an embodiment, a moiety represented by

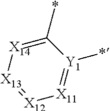

in Formula 1 may be selected from groups represented by Formulae CY1-1 to CY1-16, and
a moiety represented by

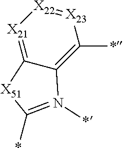

in Formula 1 may be selected from groups represented by Formulae CY2-1 to CY2-5:

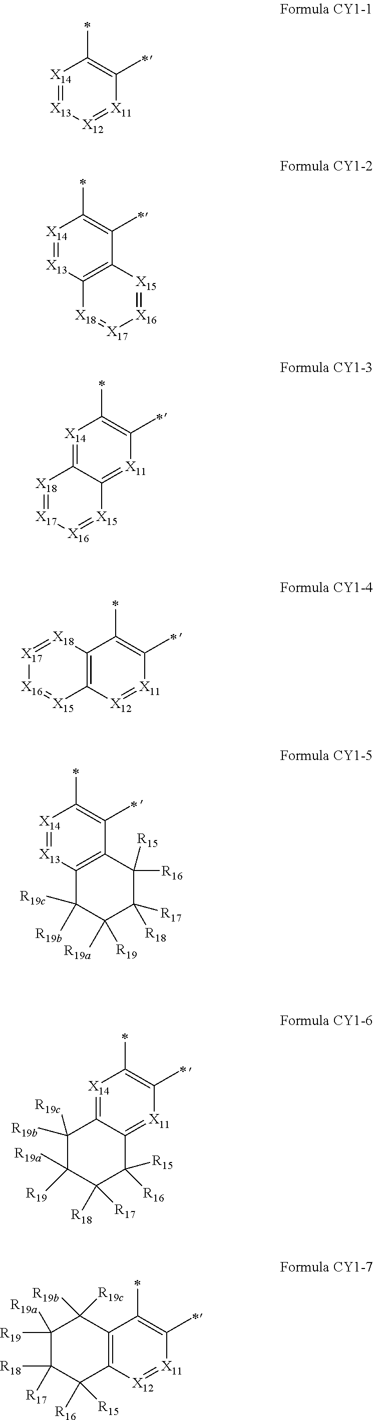

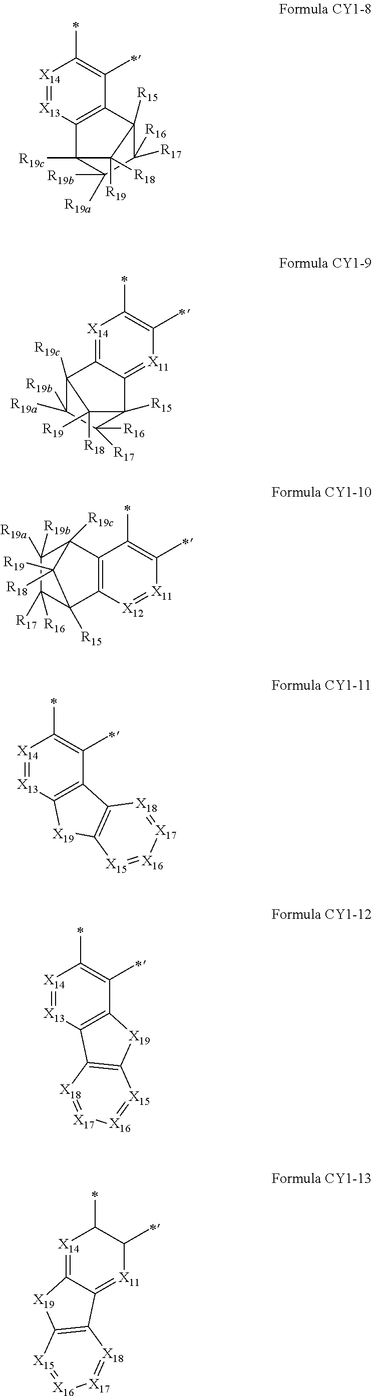

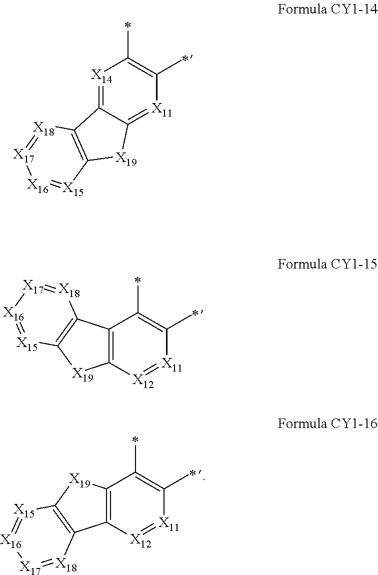

In Formulae CY1-1 to CY1-16,
X₁₁ may be N or C-[(L₁₁)_{c11}-(R₁₁)ₐ₁₁], X₁₂ may be N or C-[(L₁₂)_{c12}-(R₁₂)ₐ₁₂], X₁₃ may be N or C-[(L₁₃)_{c13}-(R₁₃)ₐ₁₃], X₁₄ may be N or C-[(L₁₄)_{c14}-(R₁₄)ₐ₁₄], X₁₅ may be N or C-[(L₁₅)_{c15}-(R₁₅)ₐ₁₅], X₁₆ may be N or C-[(L₁₆)_{c16}-(R₁₆)ₐ₁₆], X₁₇ may be N or C-[(L₁₇)_{c17}-(R₁₇)ₐ₁₇], and X₁₈ may be N or C-[(L₁₈)_{c18}-(R₁₈)ₐ₁₈],
X₁₉ may be C(R₁₉ₐ)(R_{19b}), N-[(L₁₉)_{c19}-(R₁₉)ₐ₁₉], O, S, or Si(R₁₉ₐ)(R_{19b}),
L₁₁ to L₁₉ are each independently the same as described in connection with L₁₁,
c11 to c19 are each independently the same as described in connection with c11,
R₁₁ to R₁₉ and R₁₉ₐ to R_{19c} are each independently the same as described in connection with R₁₁,
a11 to a19 are each independently the same as described in connection with a11, and
* and *′ each indicates a binding site to a neighboring atom:

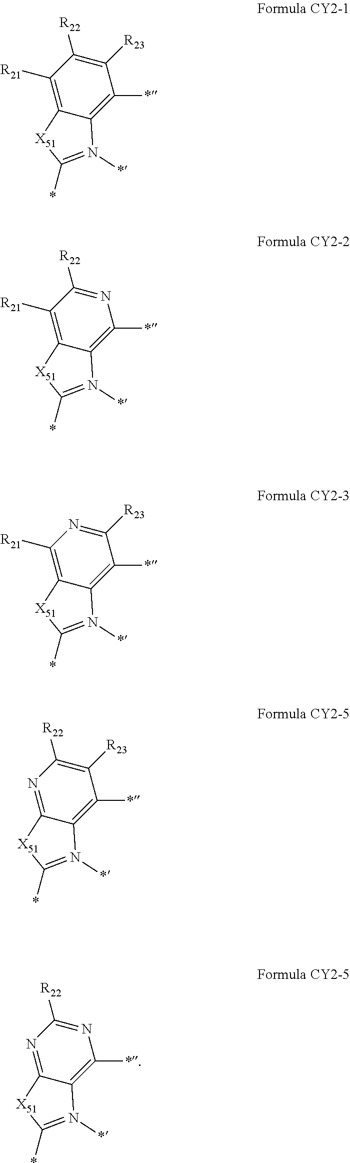

In Formulae CY2-1 to CY2-5,
X₅₁ is the same as described in connection with X₅₁,
R₂₁ to R₂₃ are each independently the same as described in connection with R₂₁, and
*, *′, and *″ each indicate a binding site to a neighboring atom.

For example,
1) a moiety represented by

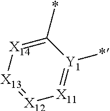

in Formula 1 may be selected from groups represented by Formulae CY1-1 to CY1-16, provided that i) at least one of X₁₁ to X₁₄ in Formula CY1-1 is N, at least one of X₁₃ and X₁₄ in Formulae CY1-2, CY1-5, CY1-8, CY1-11, and CY1-12 is N, at least one of X₁₁ and X₁₄ in Formulae CY1-3, CY1-6, CY1-9, CY1-13, and CY1-14 is N, and at least one of X₁₁ and X₁₂ in Formulae CY1-4, CY1-7, CY1-10, CY1-15, and CY1-16 is N, and ii) a moiety represented by

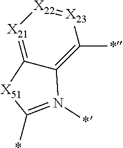

may be a group represented by Formula CY2-1;
2) a moiety represented by

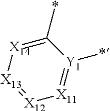

in Formula 1 may be selected from groups represented by Formulae CY1-1 to CY1-16, provided that i) all of X₁₁ to X₁₄ in Formulae CY1-1 to CY1-16 are not N, and ii) a moiety represented by

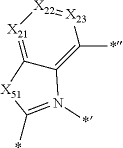

may be selected from groups represented by Formulae CY2-2 to CY2-5; or
3) a moiety represented by

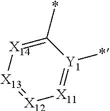

in Formula 1 may be selected from groups represented by Formulae CY1-1 to CY1-16, provided that i) at least one of X₁₁ to X₁₄ in Formula CY1-1 is N, at least one of X₁₃ and X₁₄ in Formulae CY1-2, CY1-5, CY1-8, CY1-11, and CY1-12 is N, at least one of X₁₁ and X₁₄ in Formulae CY1-3, CY1-6, CY1-9, CY1-13, and CY1-14 is N, and at least one of X₁₁ and X₁₂ in Formulae CY1-4, CY1-7, CY1-10, CY1-15, and CY1-16 is N, and ii) a moiety represented by

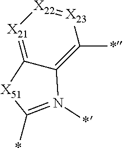

may be selected from groups represented by Formulae CY2-2 to CY2-5, but embodiments of the present disclosure are not limited thereto.

In one or more embodiments,
a moiety represented by

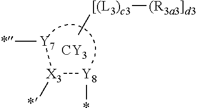

in Formula 1 may be selected from groups represented by Formulae CY3-1 to CY3-22:

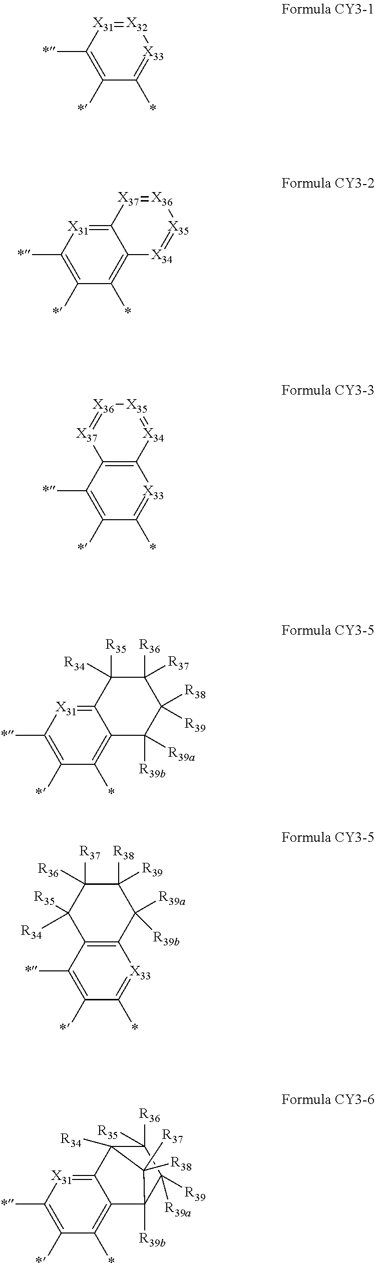

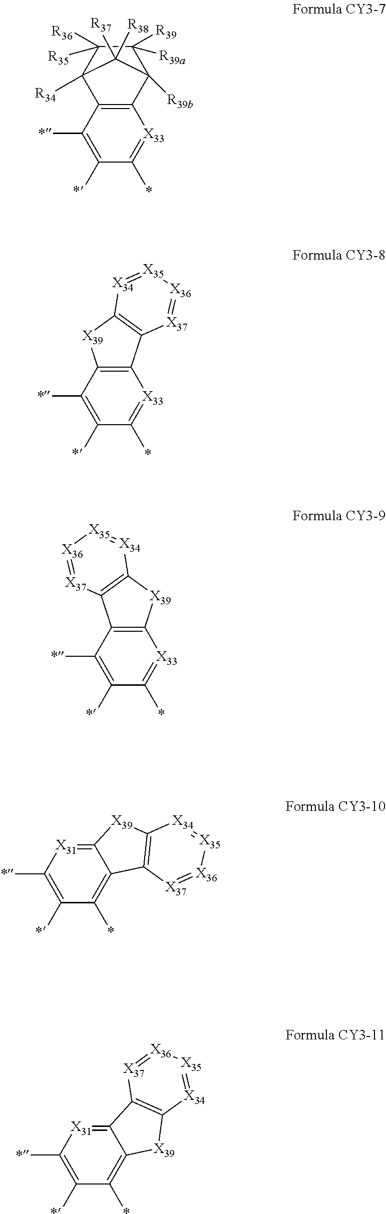

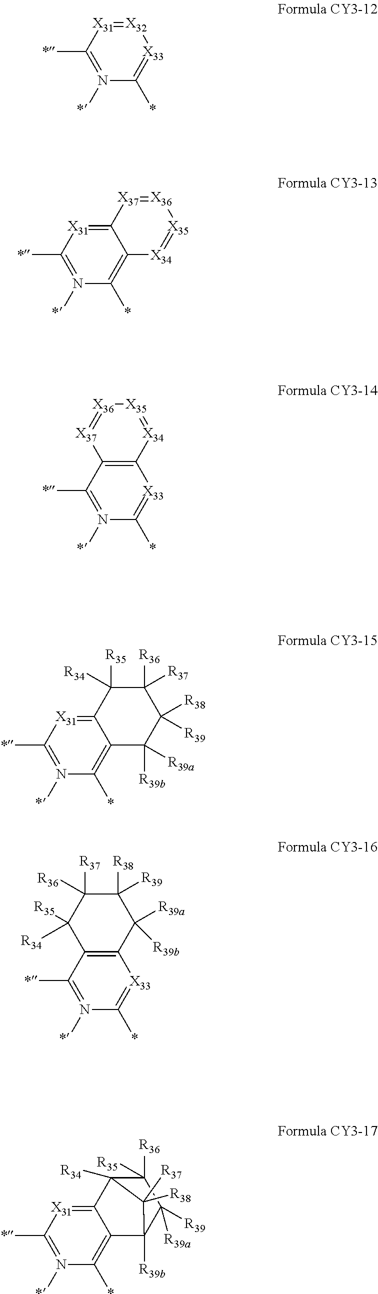

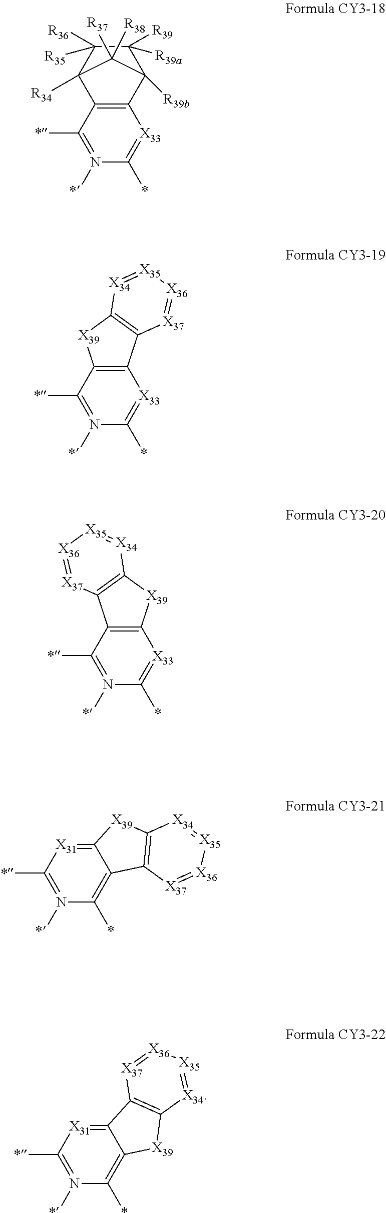

In Formulae CY3-1 to CY3-22,
X₃₁ may be N or C-[(L₃₁)_{c31}-(R₃₁)ₐ₃₁], X₃₂ may be N or C-[(L₃₂)_{c32}-(R₃₂)ₐ₃₂], X₃₃ may be N or C-[(L₃₃)_{c33}-(R₃₃)ₐ₃₃], X₃₄ may be N or C-[(L₃₄)_{c34}-(R₃₄)ₐ₃₄], X₃₅ may be N or C-[(L₃₅)_{c35}-(R₃₅)ₐ₃₅], X₃₆ may be N or C-[(L₃₆)_{c36}-(R₃₆)ₐ₃₆], and X₃₇ may be N or C-[(L₃₇)_{c37}-(R₃₇)ₐ₃₇],
X₃₉ may be C(R₃₉ₐ)(R_{39b}), N-[(L₃₉)_{c39}-(R₃₉)ₐ₃₉], O, S, or Si(R₃₉ₐ)(R_{39b}),
L₃₁ to L₃₉ are each independently the same as described in connection with L₃, c31 to c39 are each independently the same as described in connection with c3,
R₃₁ to R₃₉ and R₃₉ₐ to R_{39b} are each independently the same as described in connection with R₃,
a31 to a39 are each independently the same as described in connection with a3, and
*, *′, and *″ each indicate a binding site to a neighboring atom.

In one or more embodiments,

a moiety represented by

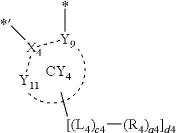

in Formula 1 may be selected from groups represented by Formulae CY4-1 to CY4-8:

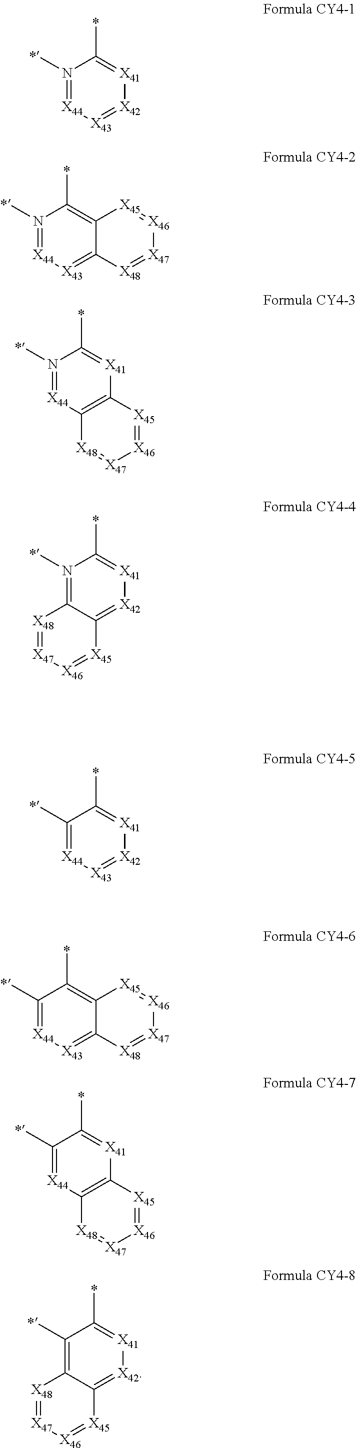

In Formulae CY4-1 to CY4-8,
X₄₁ may be N or C-[(L₄₁)_{c41}-(R₄₁)ₐ₄₁], X₄₂ may be N or C-[(L₄₂)_{c42}-(R₄₂)ₐ₄₂], X₄₃ may be N or C-[(L₄₃)_{c43}-(R₄₃)ₐ₄₃], X₄₄ may be N or C-[(L₄₄)_{c44}-(R₄₄)ₐ₄₄], X₄₅ may be N or C-[(L₄₅)_{c45}-(R₄₅)ₐ₄₅], X₄₆ may be N or C-[(L₄₆)_{c46}-(R₄₆)ₐ₄₆], X₄₇ may be N or C-[(L₄₇)_{c47}-(R₄₇)ₐ₄₇], and X₄₈ may be N or C-[(L₄₈)_{c48}-(R₄₈)ₐ₄₈],
L₄₁ to L₄₈ are each independently the same as described in connection with L₄,
c41 to c48 are each independently the same as described in connection with c4,
R₄₁ to R₄₈ are each independently the same as described in connection with R₄,
a41 to a48 are each independently the same as described in connection with a4, and
* and *′ each indicate a binding site to a neighboring atom.

In one or more embodiments, the organometallic compound may be represented by Formula 1-1 or 1-2,
1) in Formulae 1-1 and 1-2, a moiety represented by

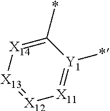

may be selected from groups represented by Formulae CY1-1(2) to CY1-1(8), and a moiety represented by

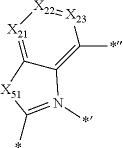

may be a group represented by Formula CY2-1;
2) in Formulae 1-1 and 1-2, a moiety represented by

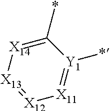

may be a group represented by Formula CY1-1(1), and a moiety represented by

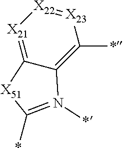

may be selected from groups represented by Formulae CY2-2 to CY2-5; or
3) in Formulae 1-1 and 1-2, a moiety represented by

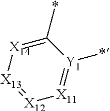

may be selected from groups represented by Formulae CY1-1(2) to CY1-1(8), and a moiety represented by

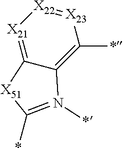

may be selected from groups represented by Formulae CY2-2 to CY2-5, but embodiments of the present disclosure are not limited thereto:

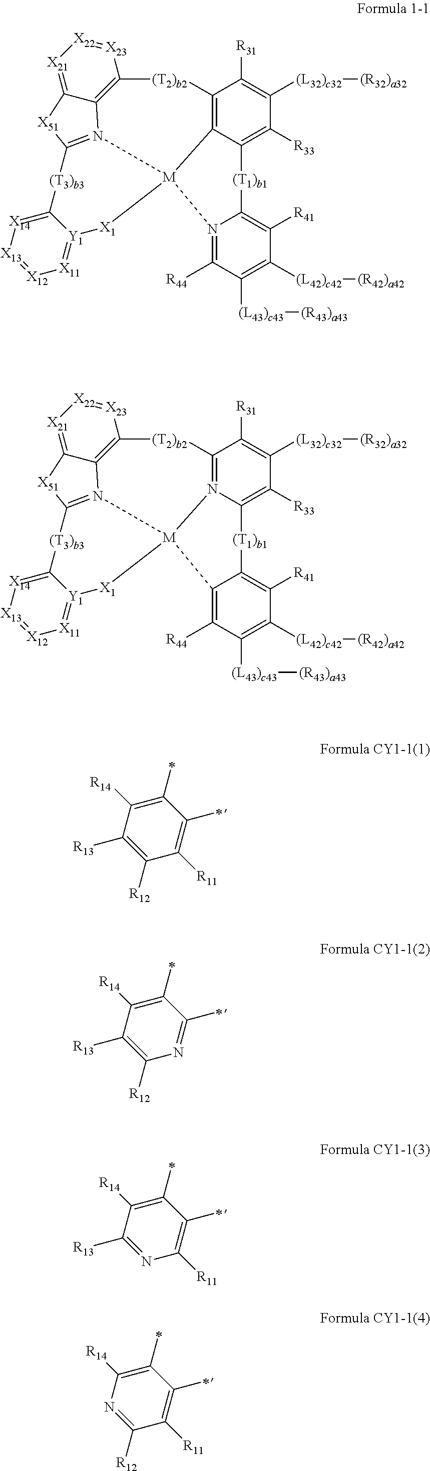

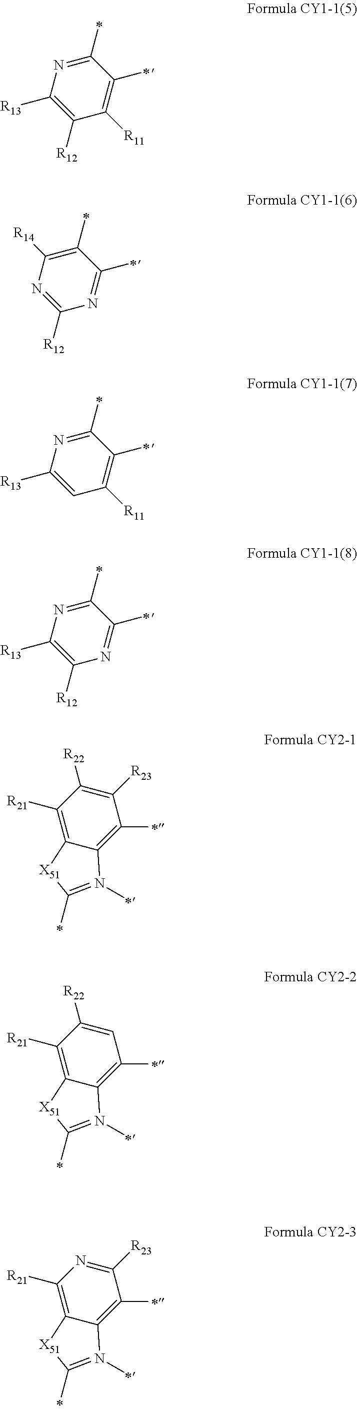

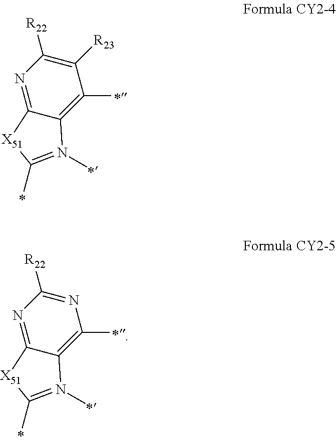

In Formulae 1-1, 1-2, CY1-1(1) to CY1-1(8), and CY2-1 to CY2-5,
M, X₁, Y₁, T₁ to T₃, b1 to b3, X₅₁, X₁₁ to X₁₄, and X₂₁ to X₂₃ are each independently the same as described herein,
L₃₂ is the same as described in connection with L₃,
c32 is the same as described in connection with c3,
R₃₁ to R₃₃ are each independently the same as described in connection with R₃,
a32 is the same as described in connection with a3,
L₄₂ and L₄₃ are each independently the same as described in connection with L₄,
c42 and c43 are each independently the same as described in connection with c4,
R₄₁ to R₄₄ are each independently the same as described in connection with R₄,
a42 and a43 are each independently the same as described in connection with a4,
R₁₁ to R₁₄ are each independently the same as described in connection with R₁₁,
R₂₁ to R₂₃ are each independently the same as described in connection with R₂₁, and
*, *′, and *″ each indicate a binding site to a neighboring atom.

For example, the organometallic compound may be one of Compounds 1 to 822, but embodiments of the present disclosure are not limited thereto:

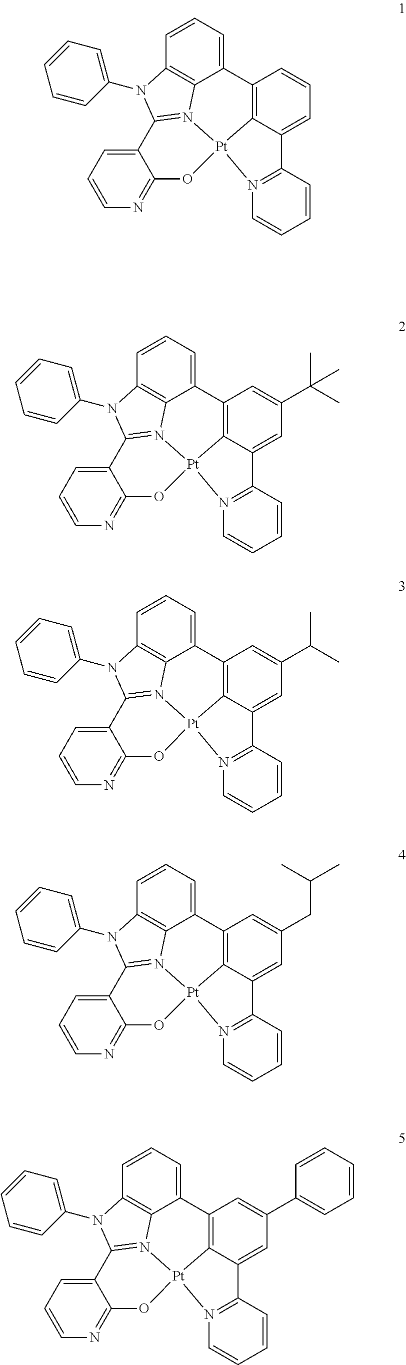

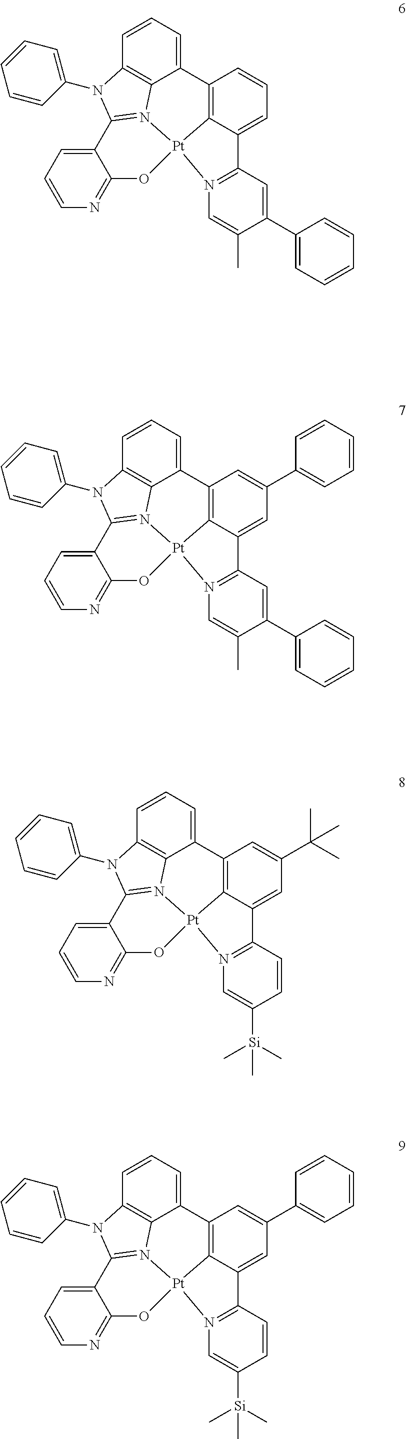

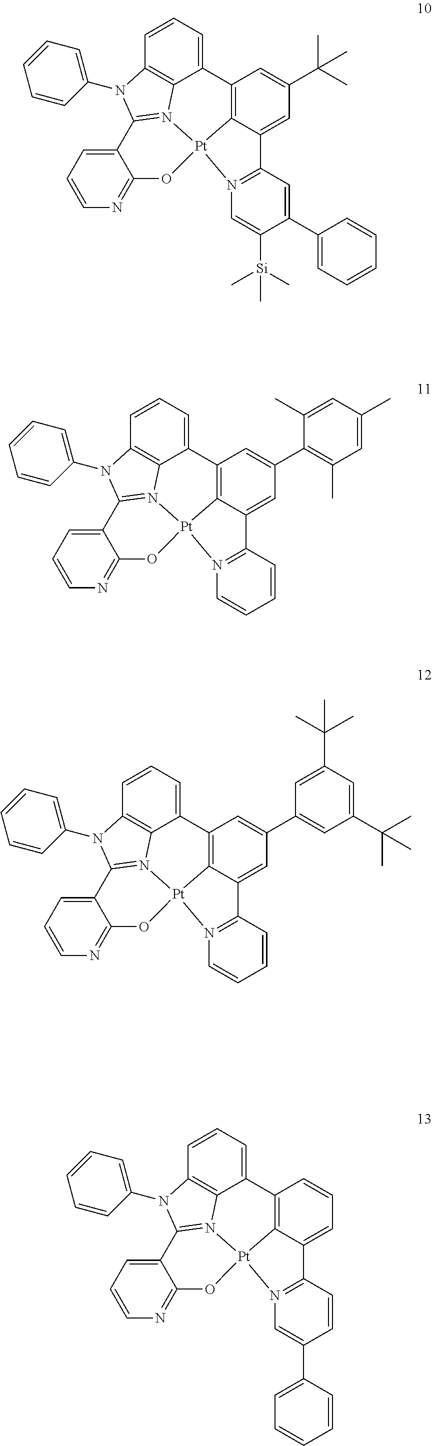

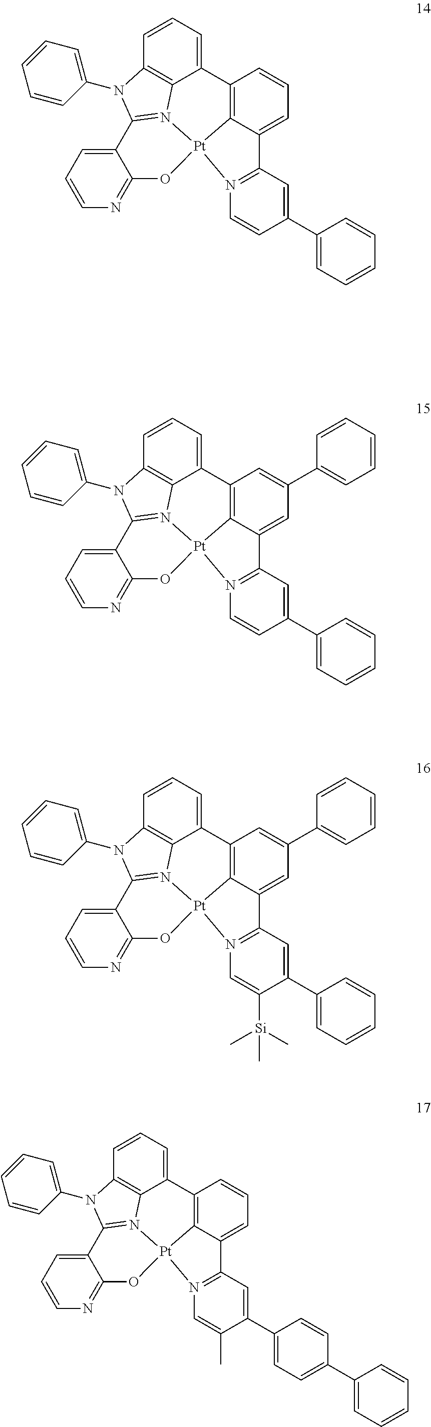

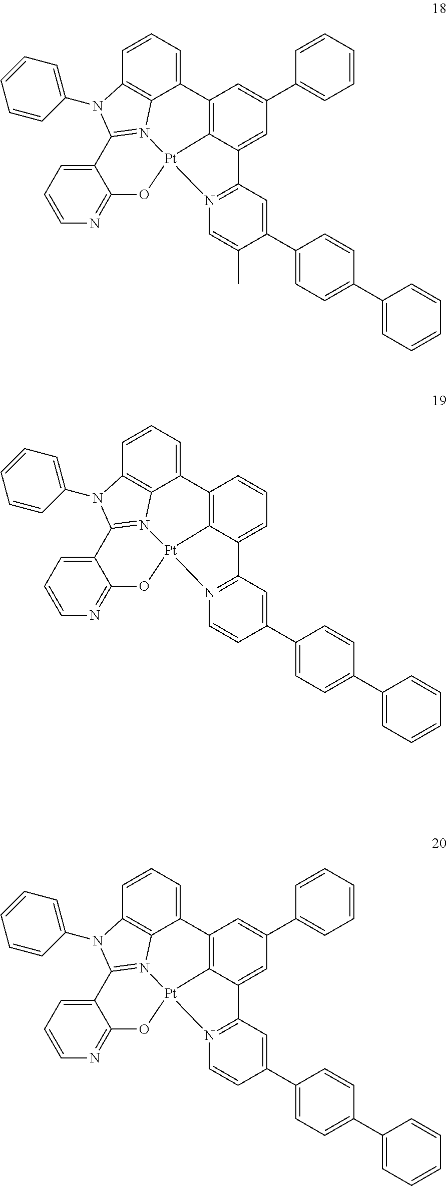

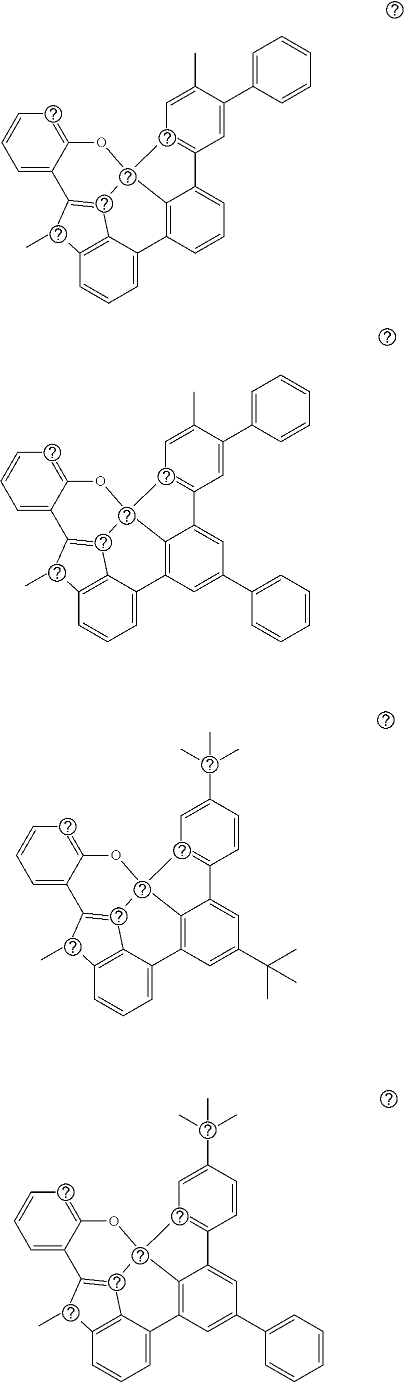 [SMILES: Cc1cc2c(cc1-c1ccccc1)-c1cccc3c1C21Oc2ccccc2C2=C1c1c-3cccc1C2C ; Cc1cc2c(cc1-c1ccccc1)-c1cc(-c3ccccc3)cc3c1C21Oc2ccccc2C2=C1c1c-3cccc1C2C ; CC1C2=C3c4c(cccc41)-c1cc(C(C)(C)C)cc4c1C3(Oc1ccccc12)c1cc(C(C)(C)C)ccc1-4 ; CC1C2=C3c4c(cccc41)-c1cc(-c4ccccc4)cc4c1C3(Oc1ccccc12)c1cc(C(C)(C)C)ccc1-4]

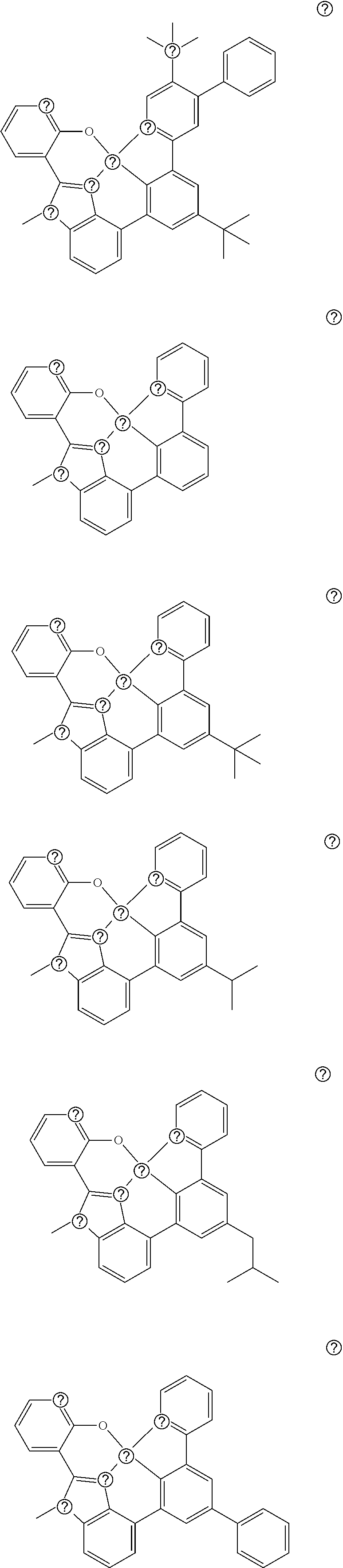 [SMILES: CC1C2=C3c4c(cccc41)-c1cc(C(C)(C)C)cc4c1C3(Oc1ccccc12)c1cc(C(C)(C)C)c(-c2ccccc2)cc1-4 ; CC1C2=C3c4c(cccc41)-c1cccc4c1C3(Oc1ccccc12)c1ccccc1-4 ; CC1C2=C3c4c(cccc41)-c1cc(C(C)(C)C)cc4c1C3(Oc1ccccc12)c1ccccc1-4 ; CC(C)c1cc2c3c(c1)-c1cccc4c1C1=C(c5ccccc5OC13c1ccccc1-2)C4C ; CC(C)Cc1cc2c3c(c1)-c1cccc4c1C1=C(c5ccccc5OC13c1ccccc1-2)C4C ; CC1C2=C3c4c(cccc41)-c1cc(-c4ccccc4)cc4c1C3(Oc1ccccc12)c1ccccc1-4]

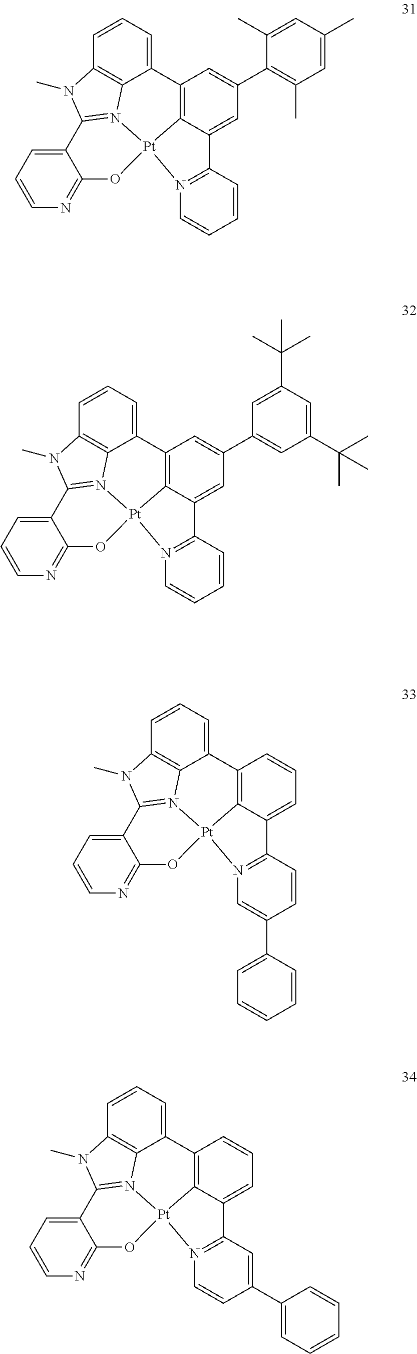

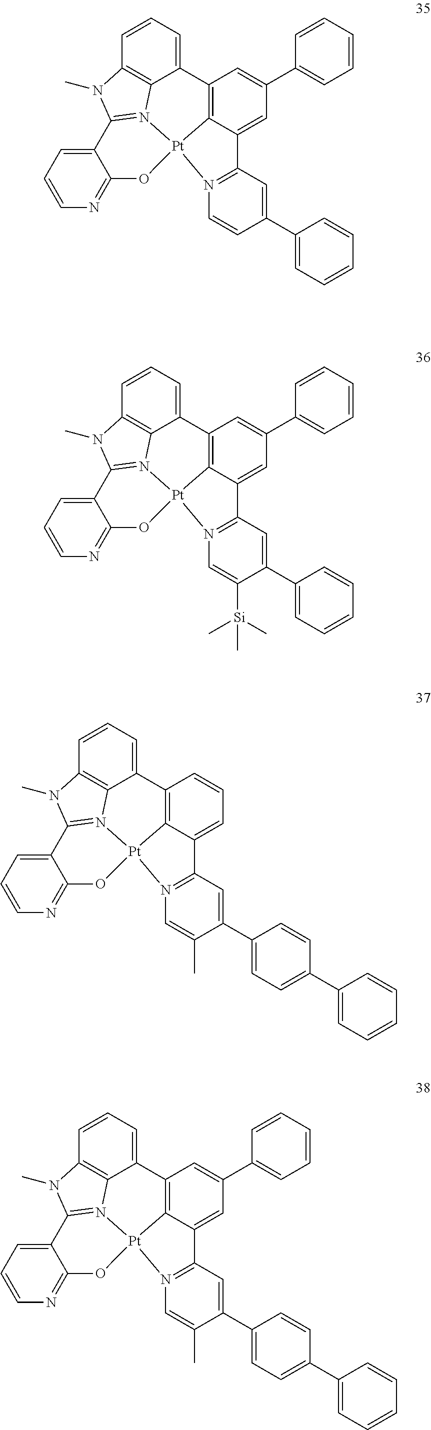

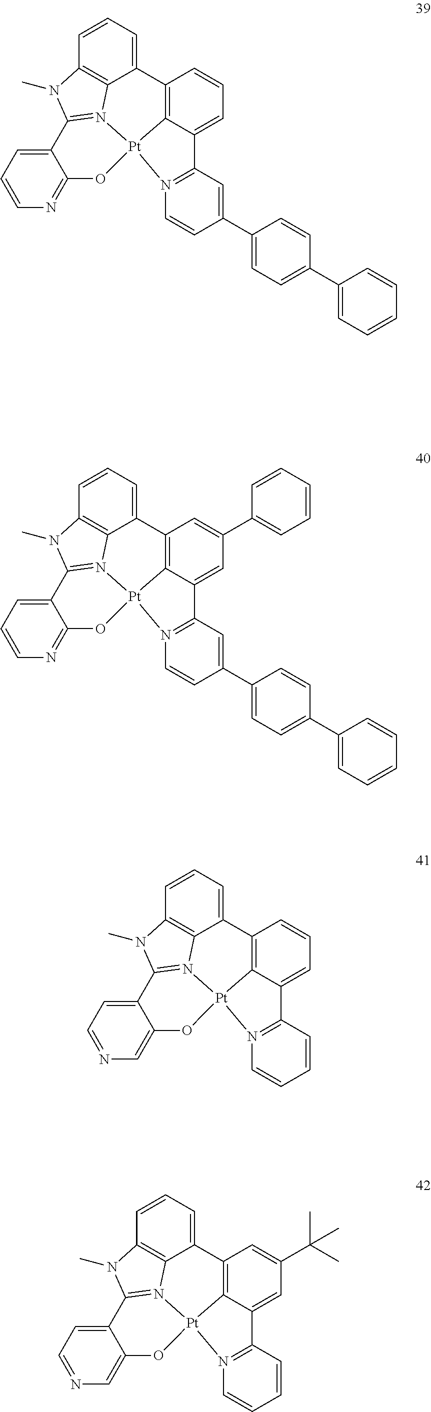

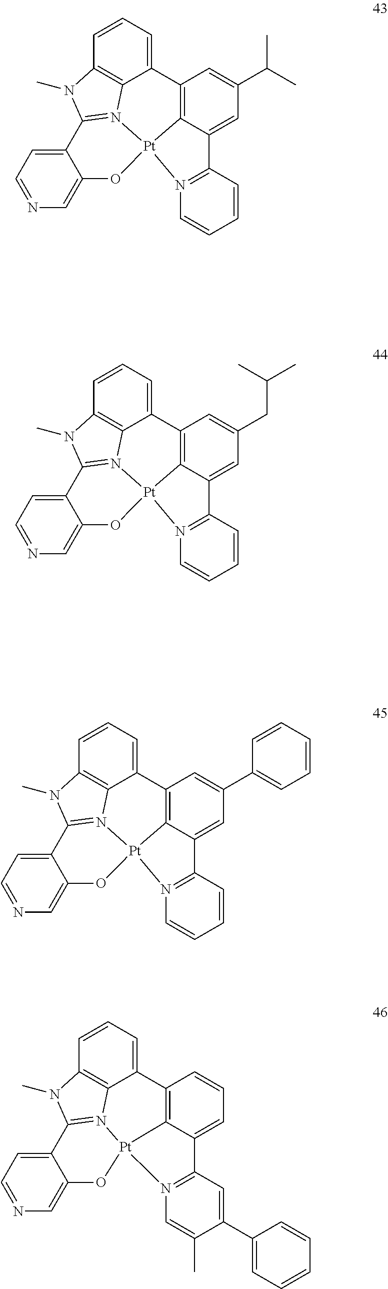

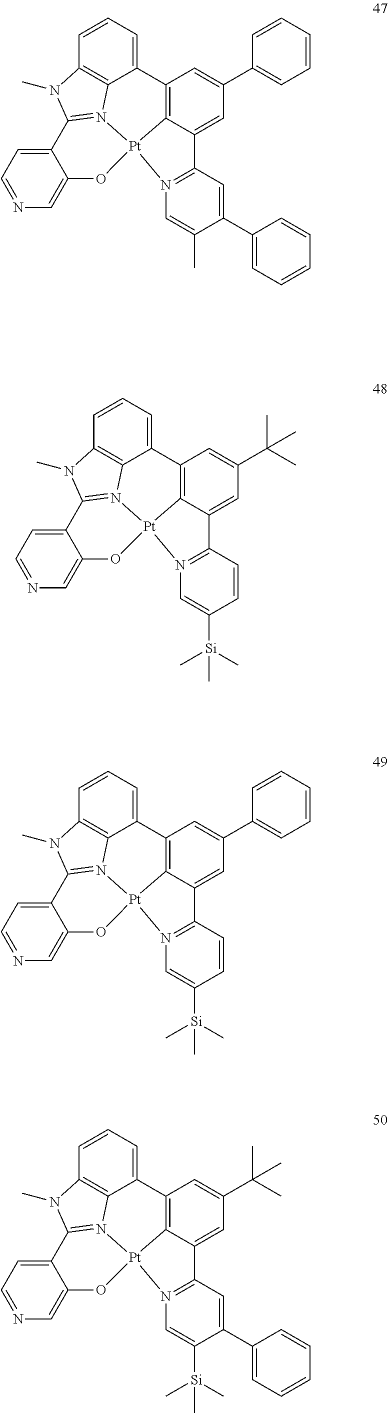

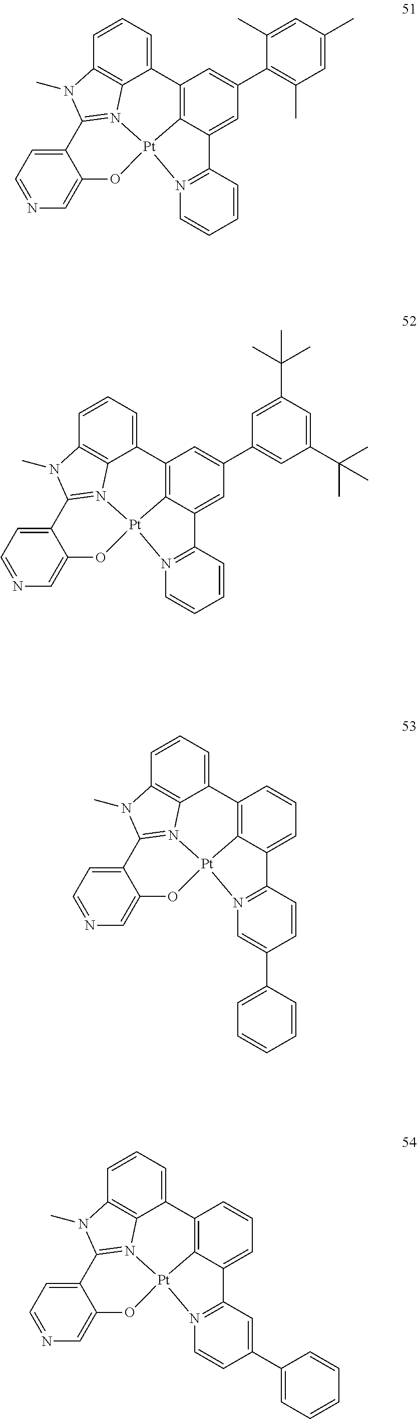

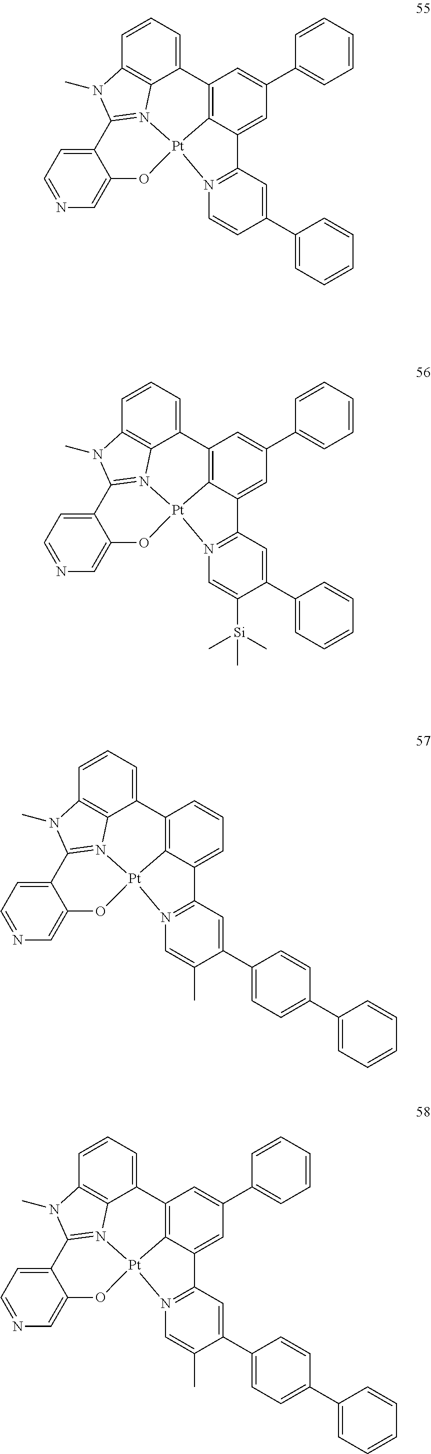

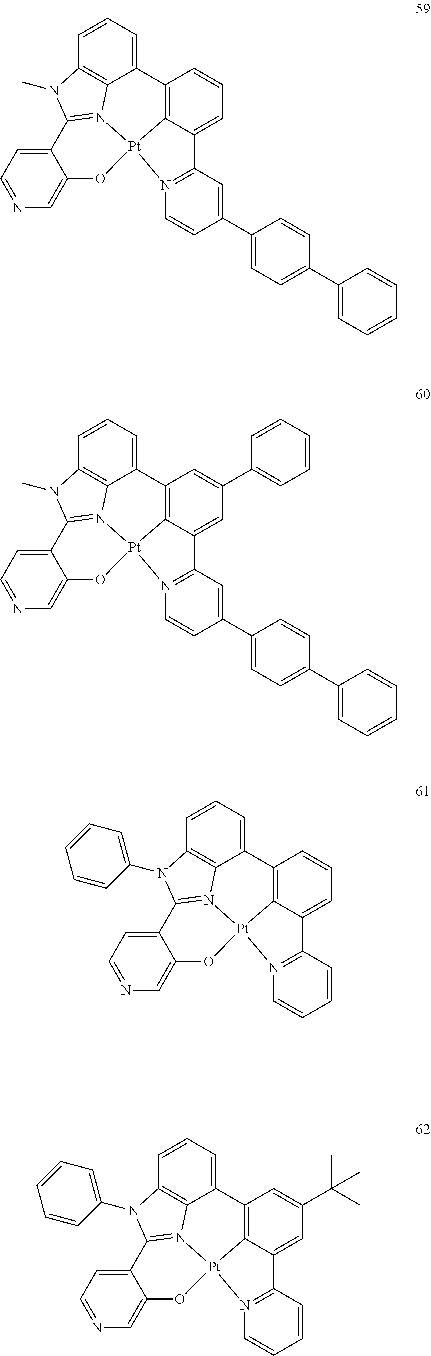

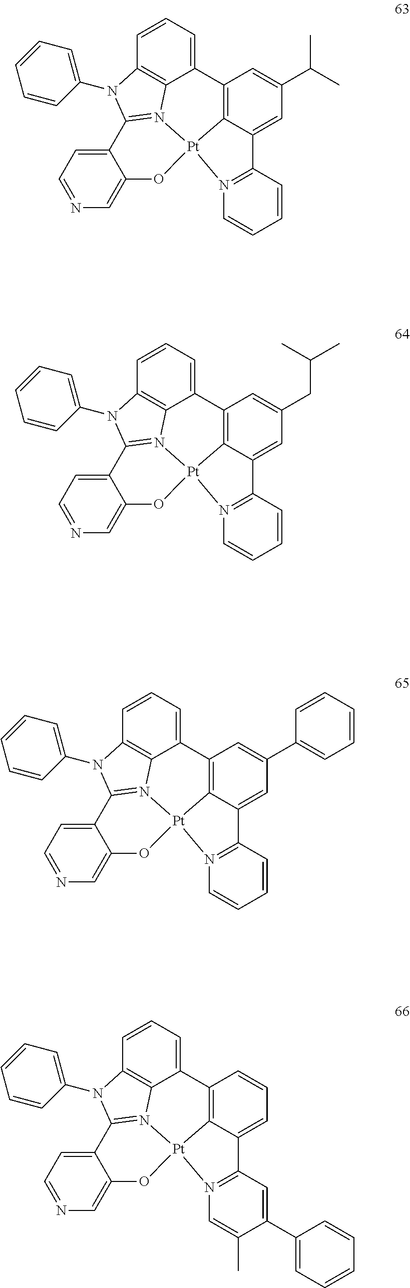

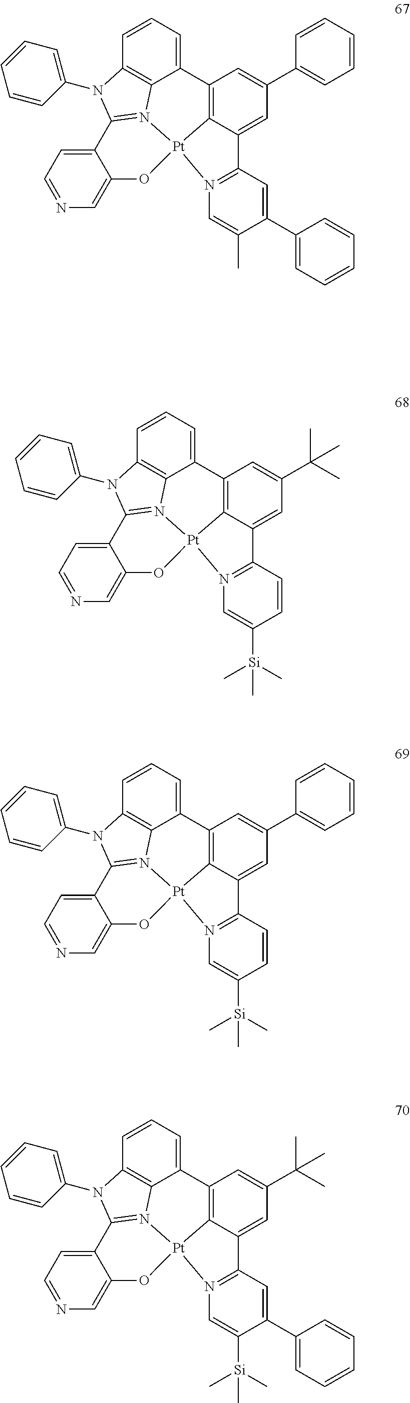

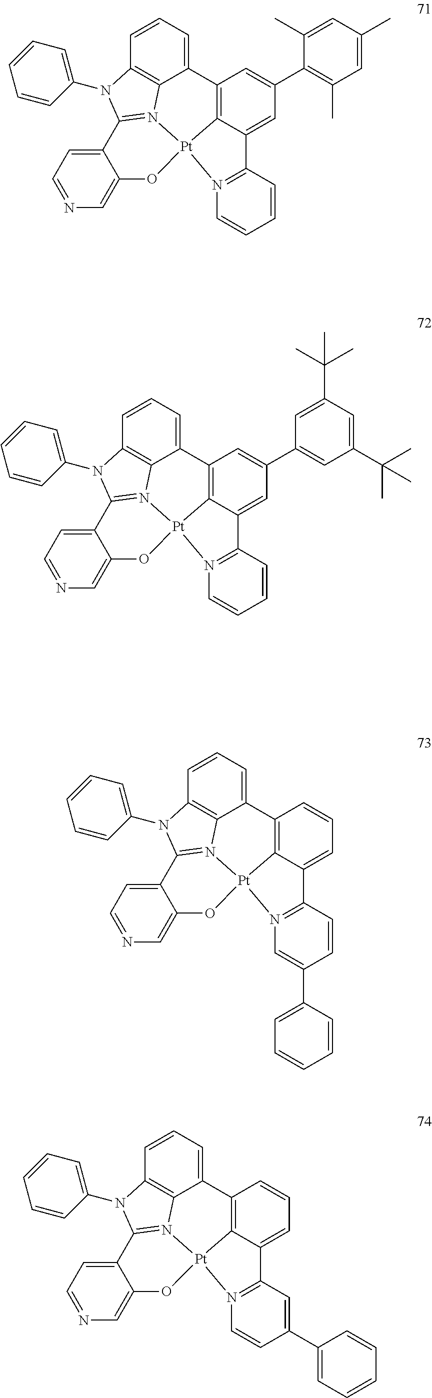

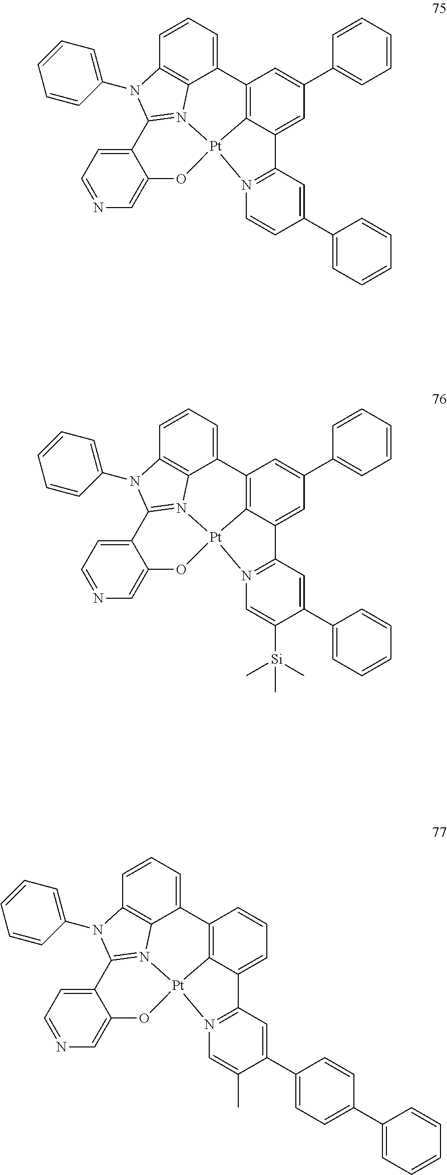

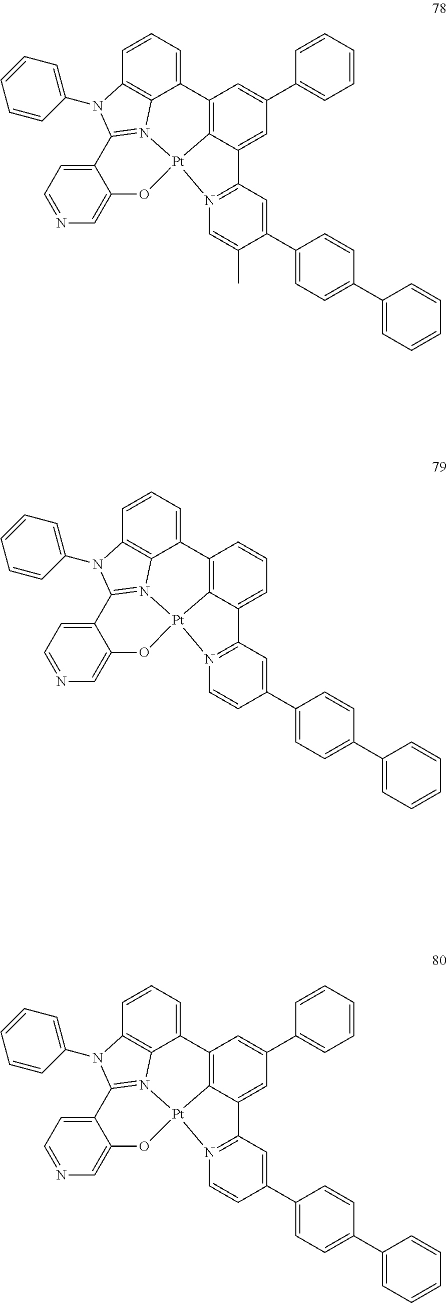

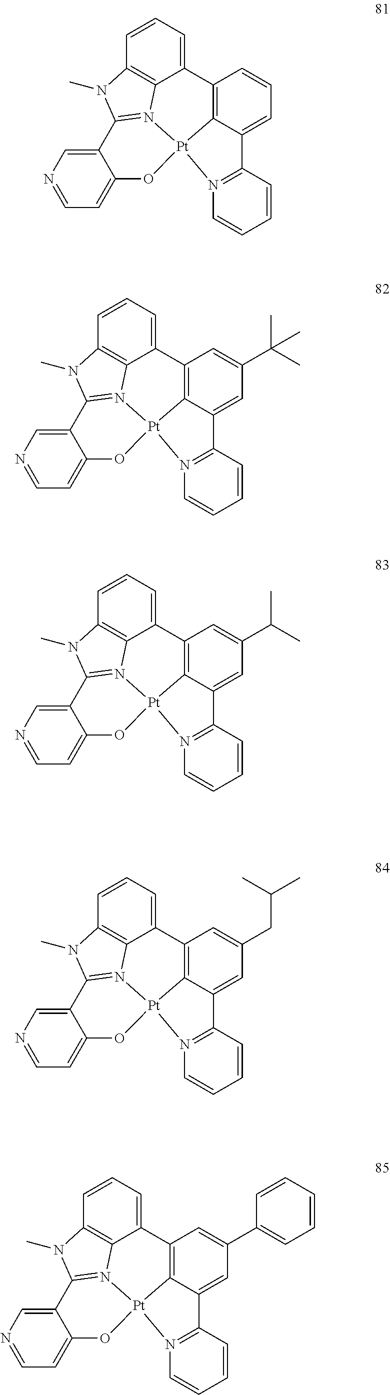

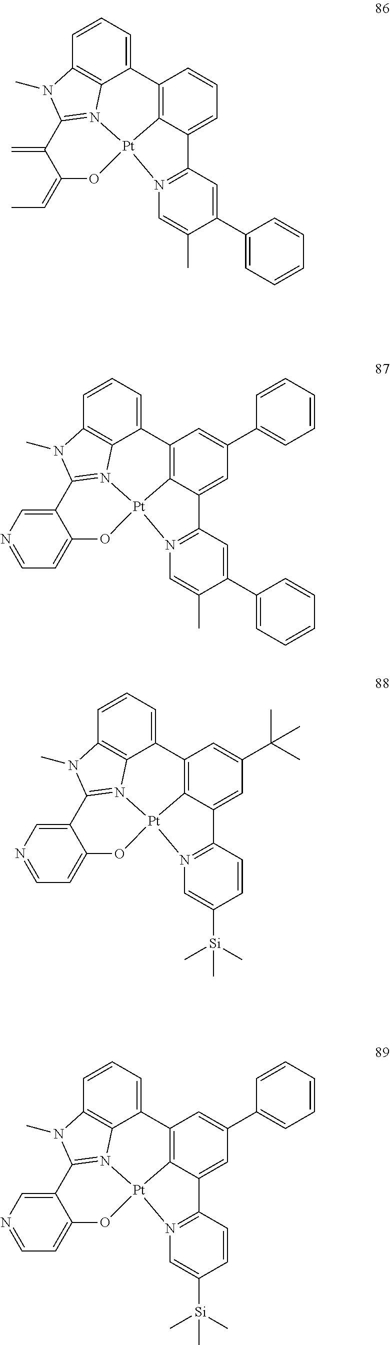

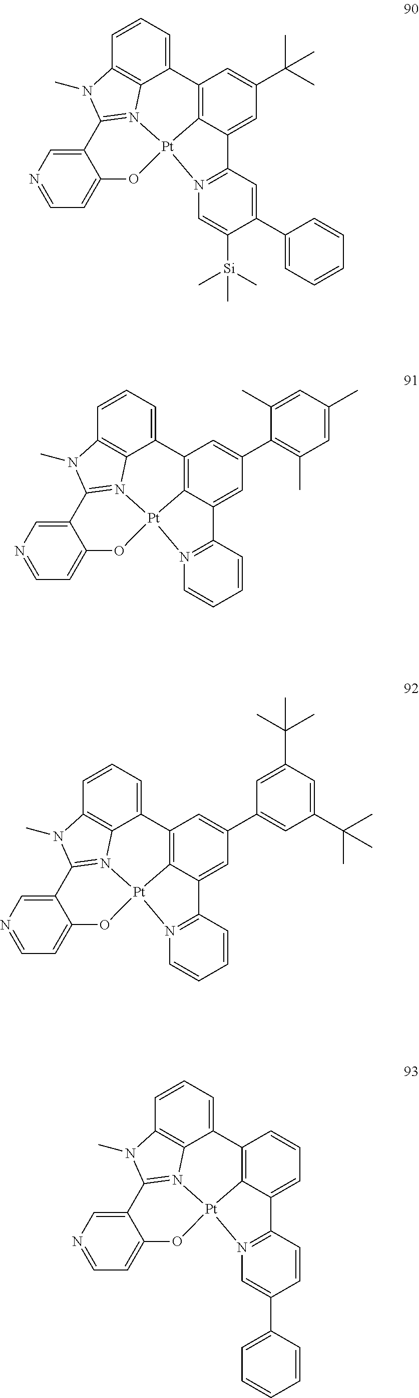

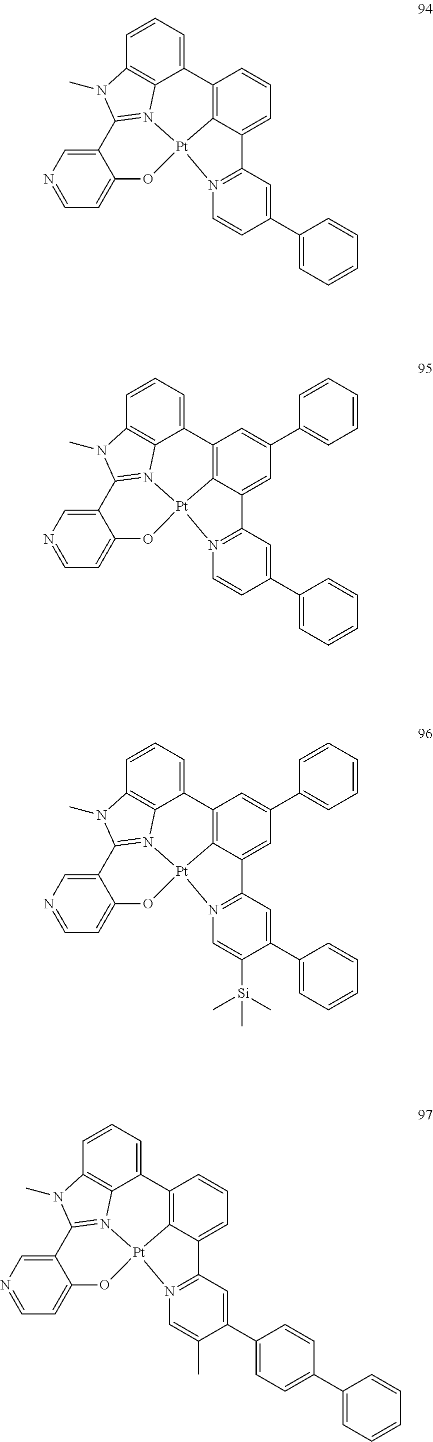

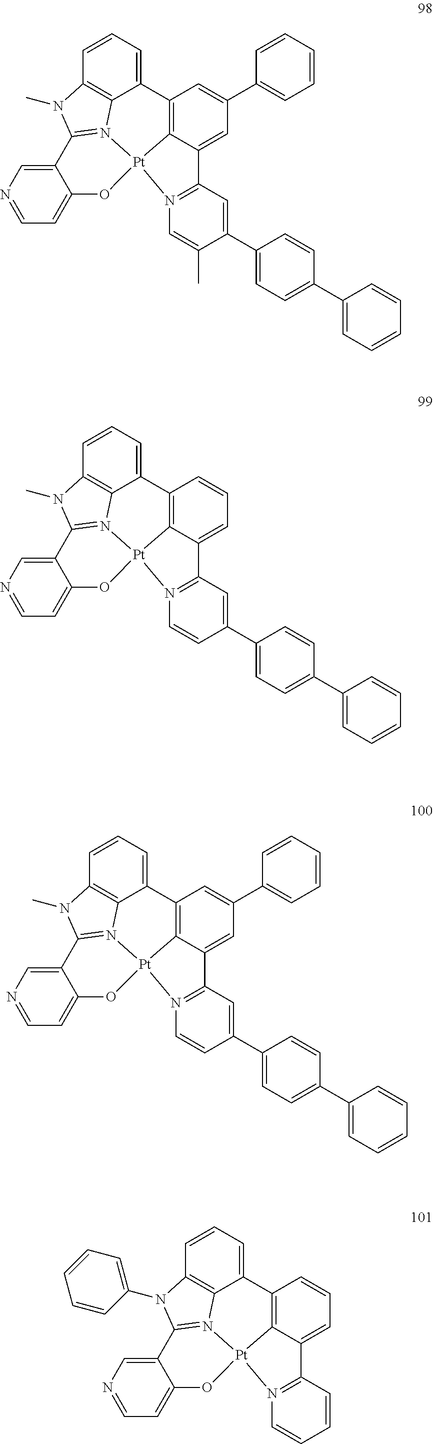

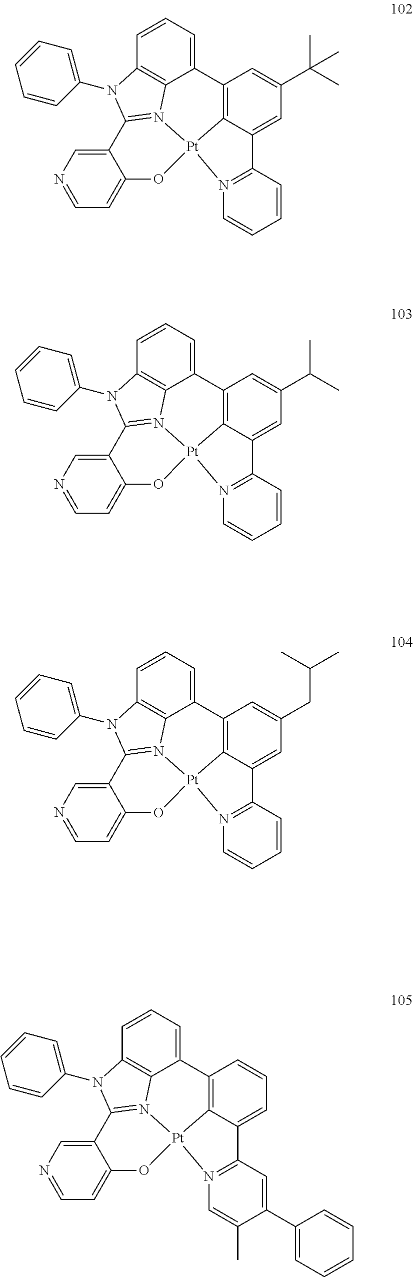

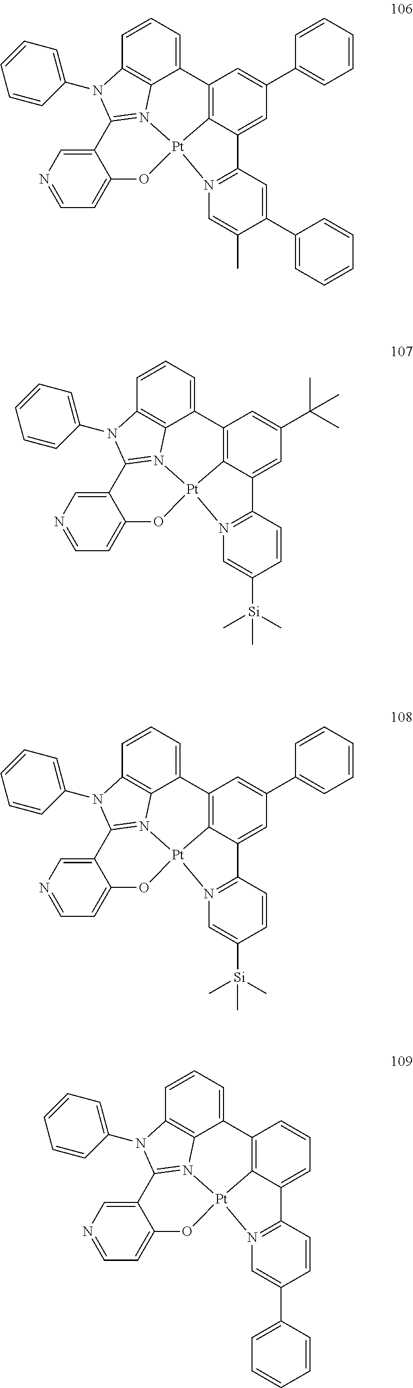

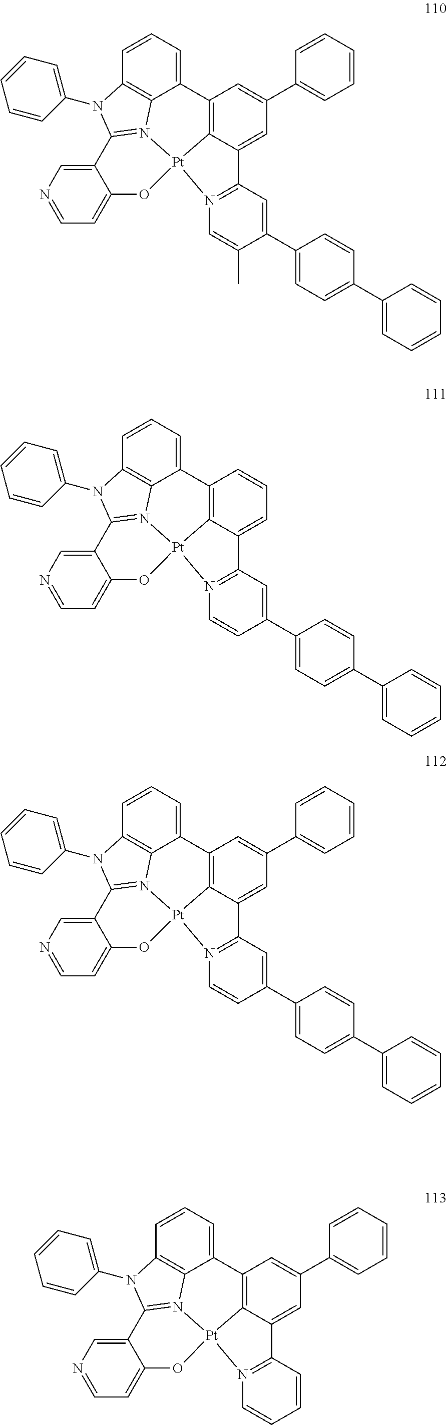

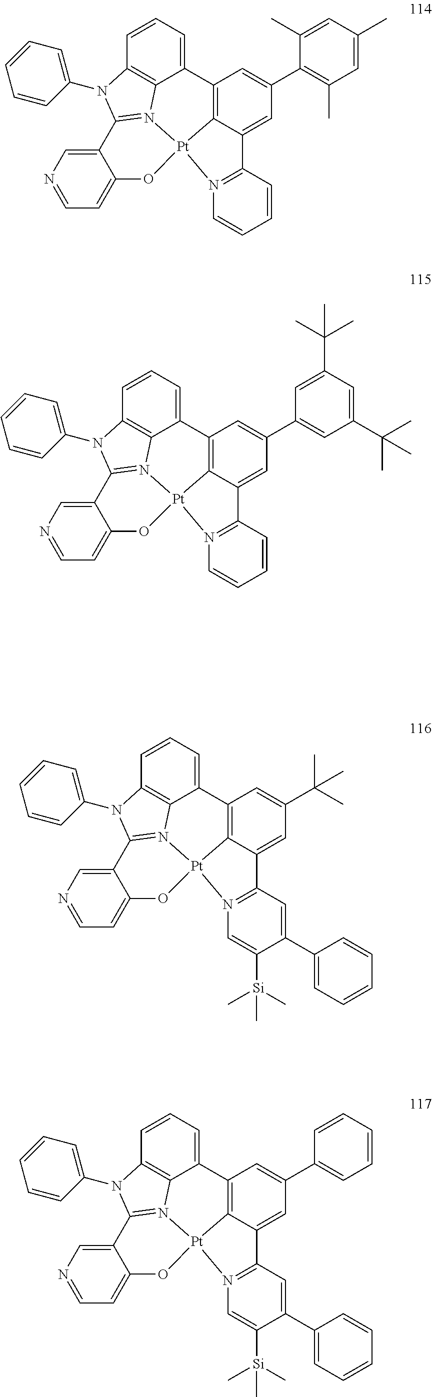

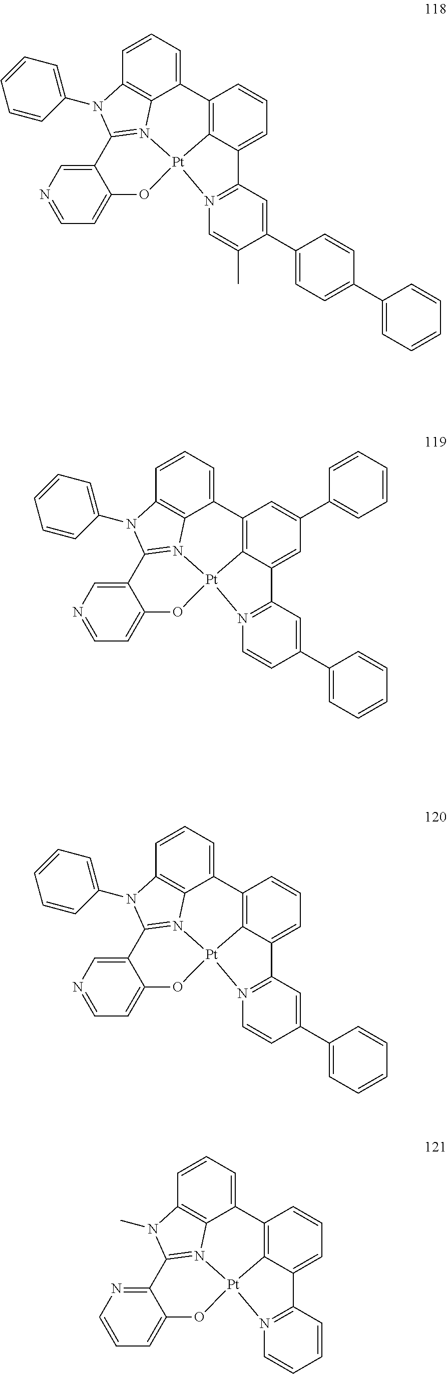

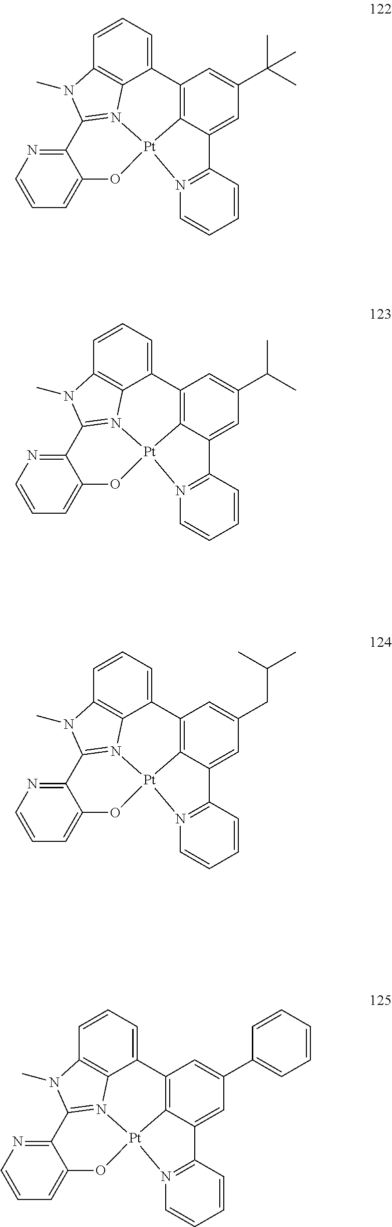

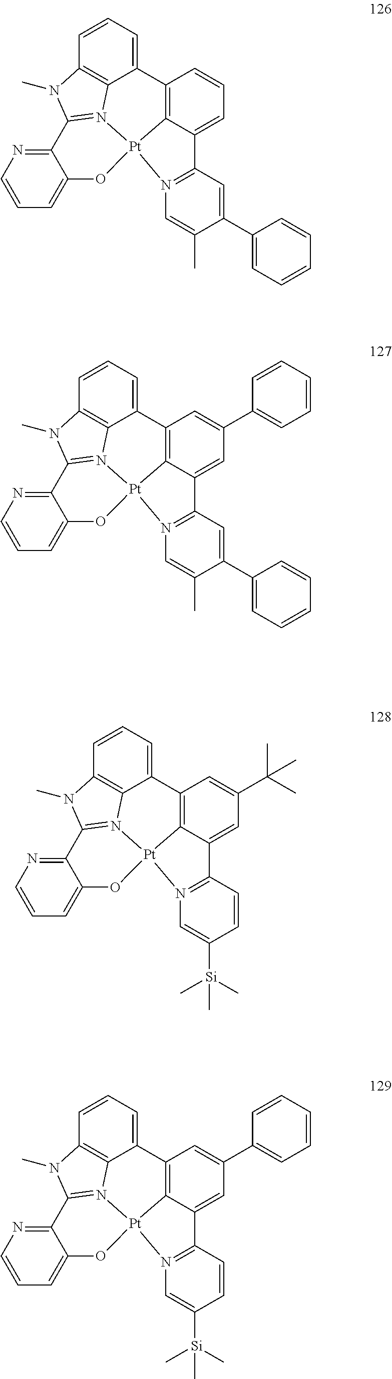

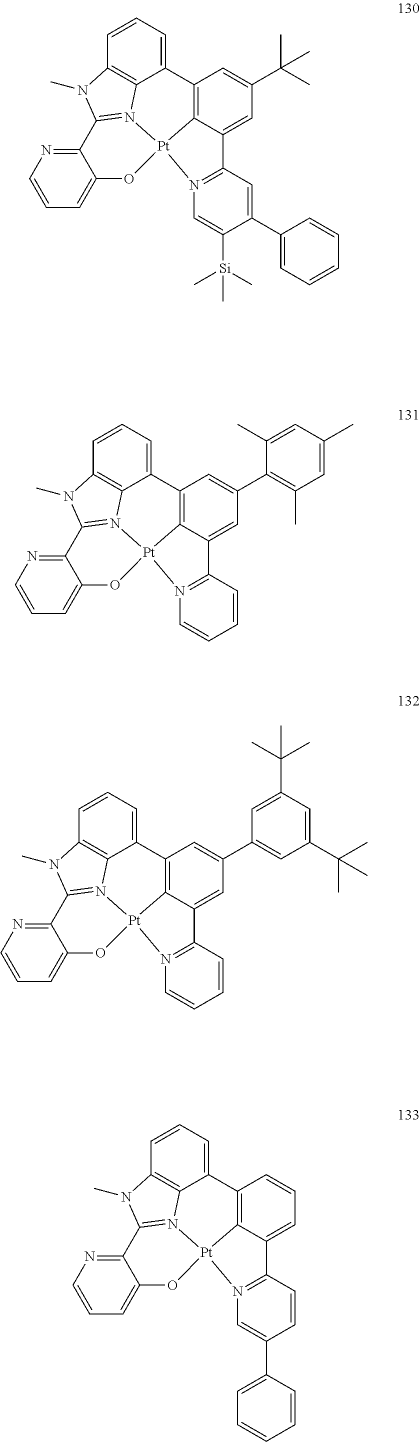

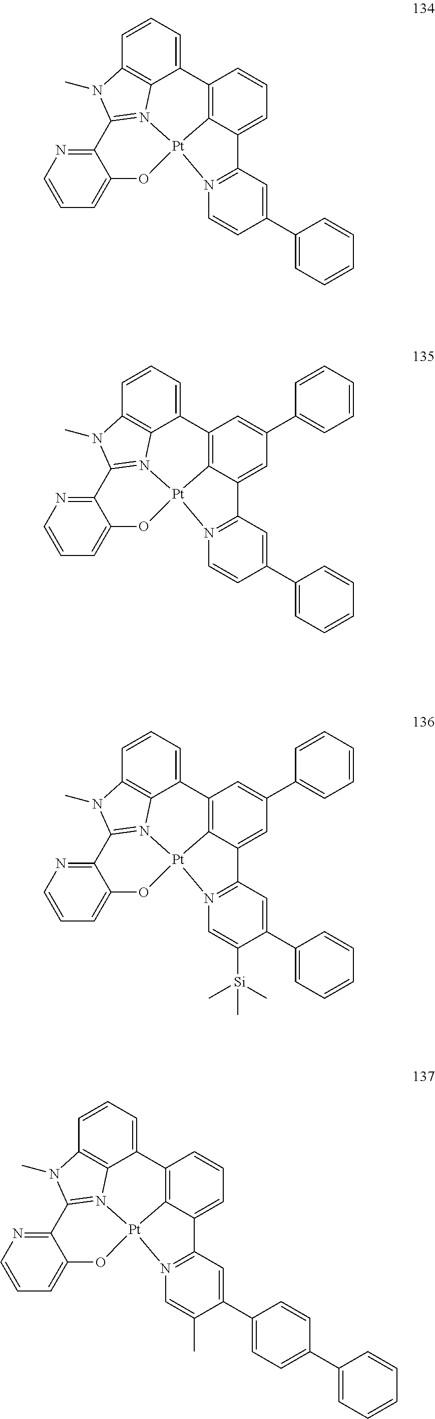

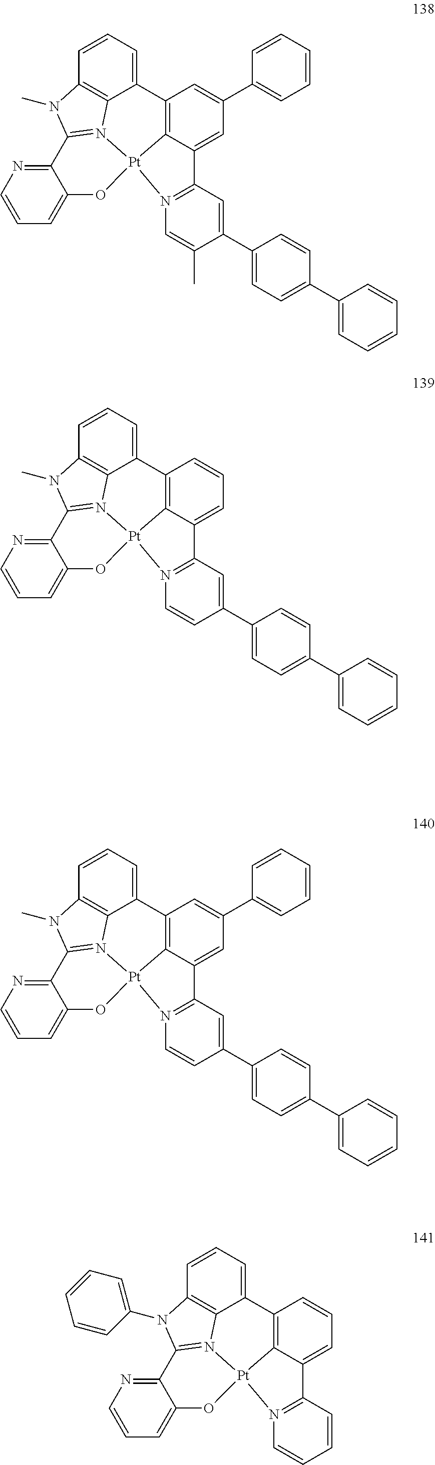

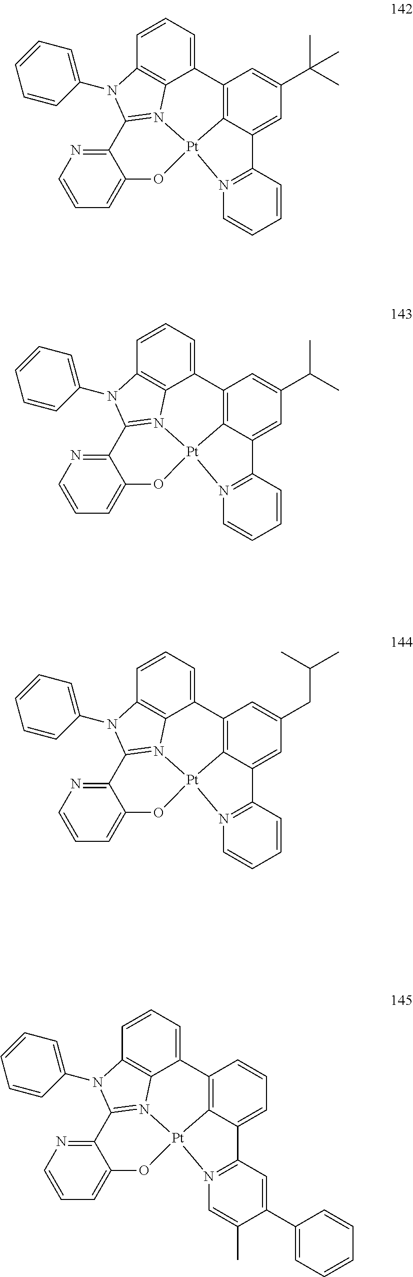

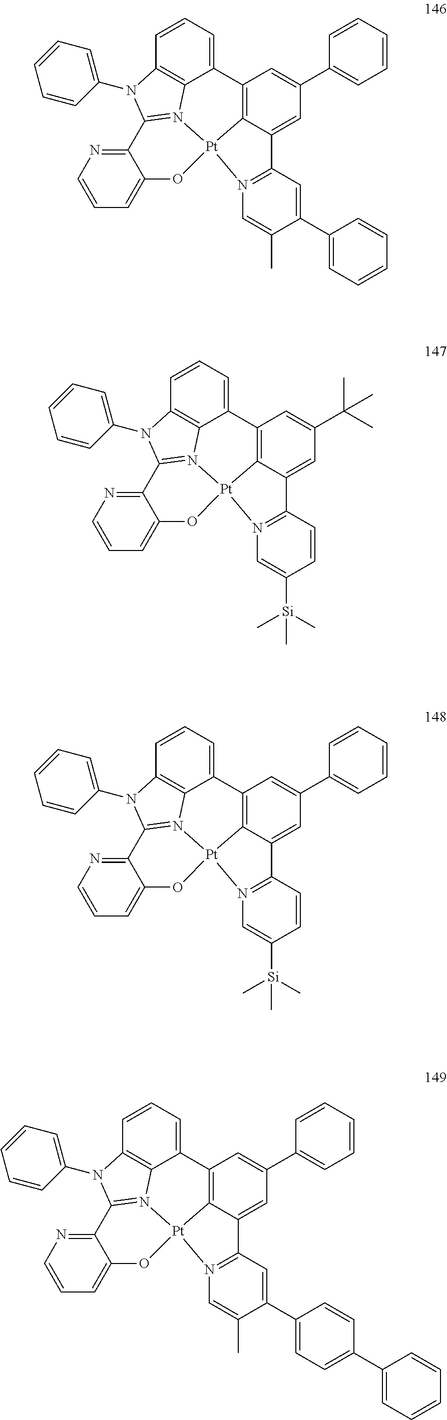

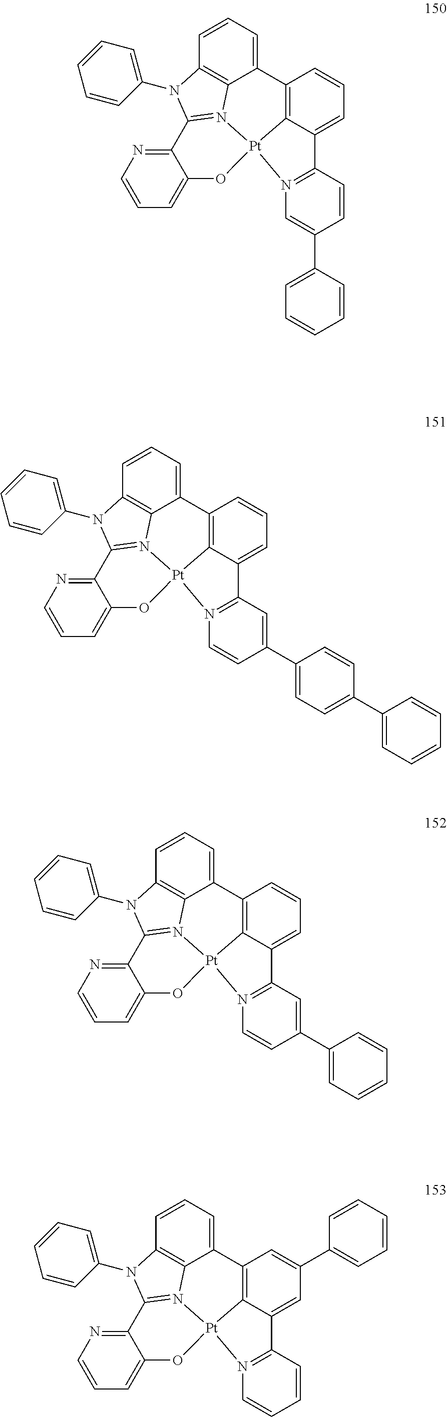

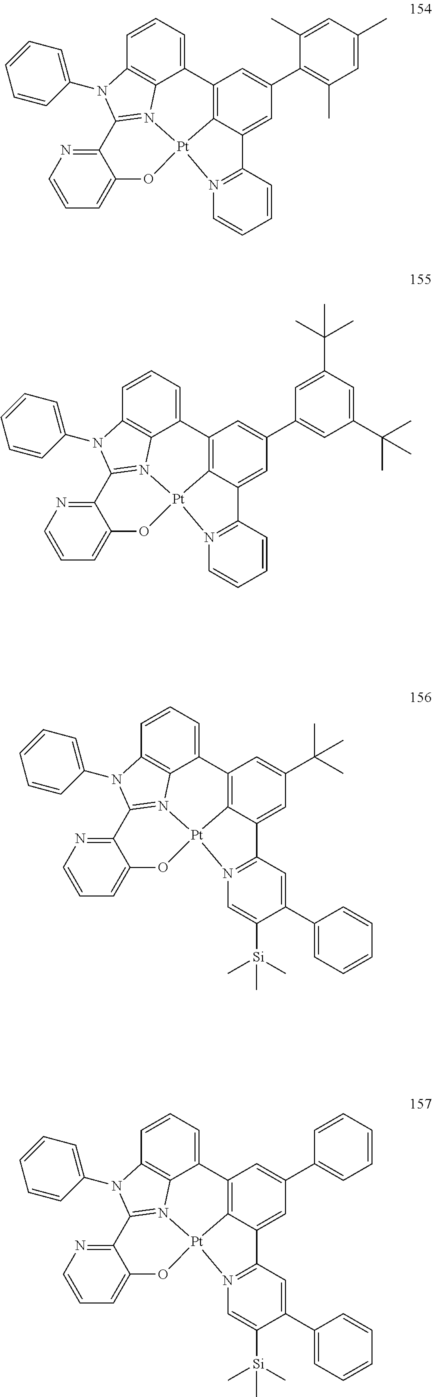

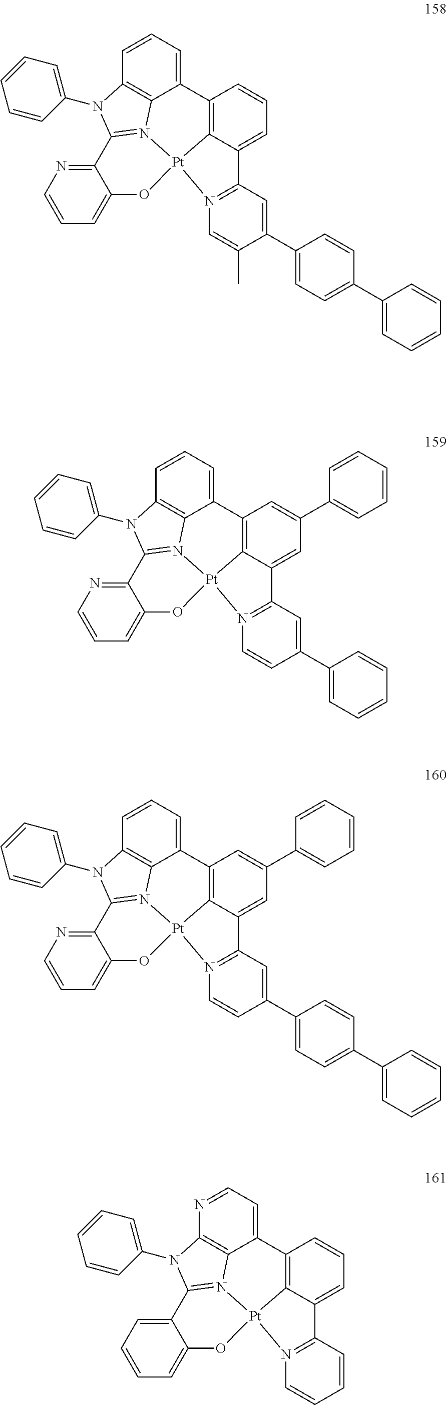

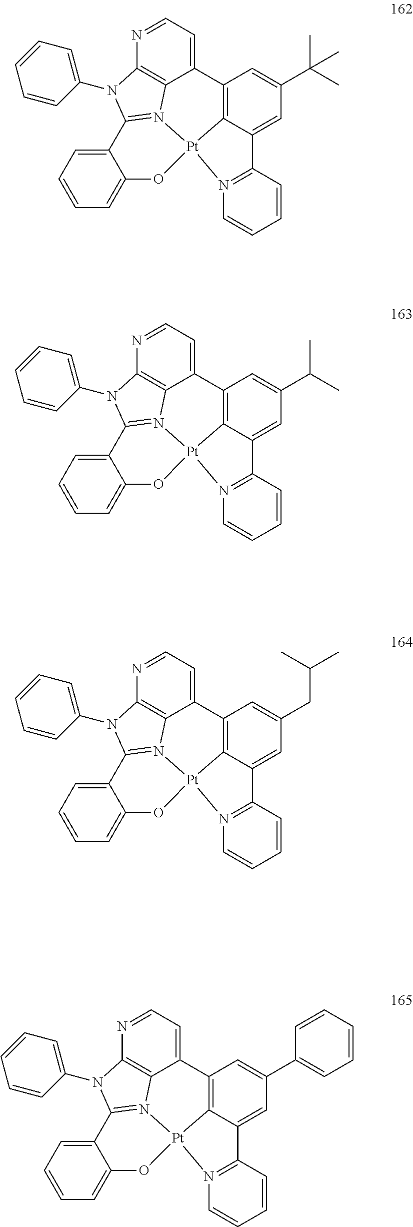

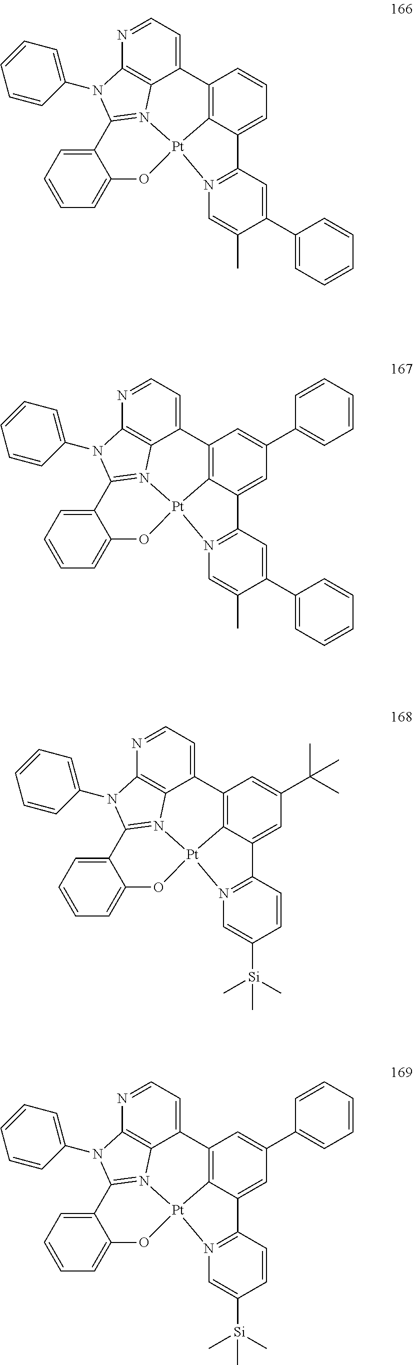

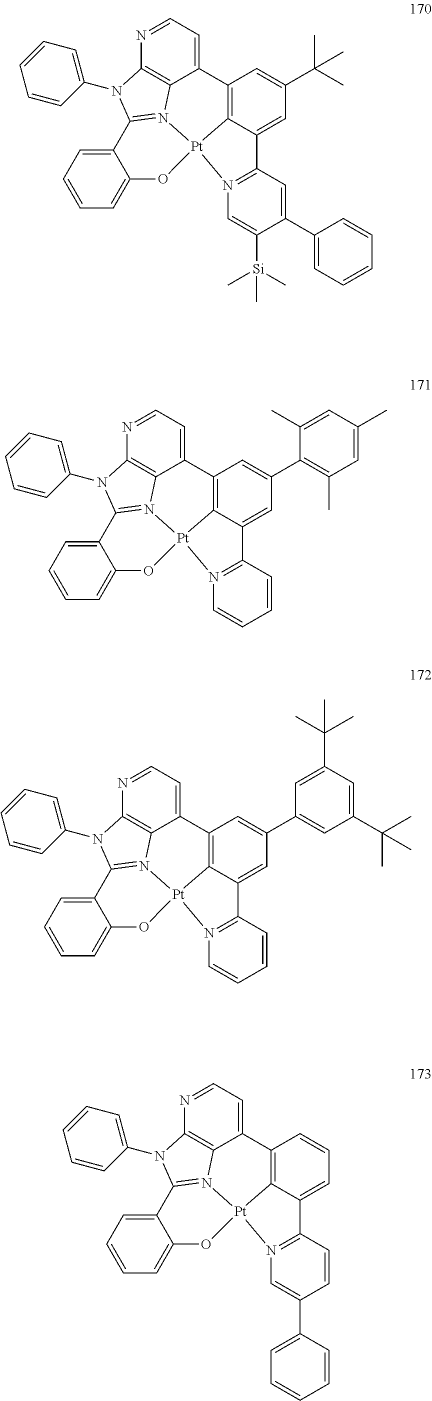

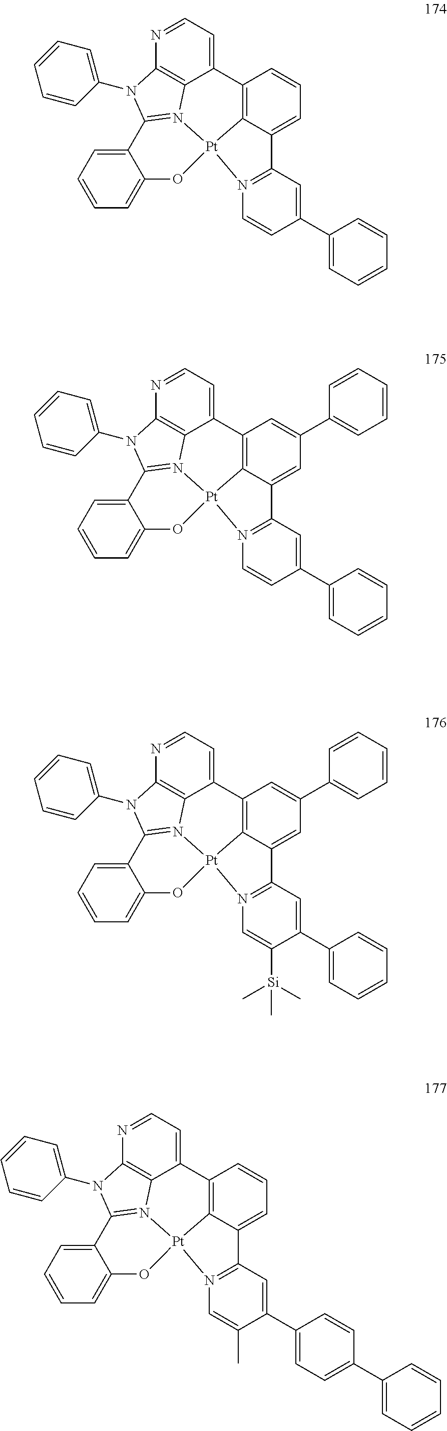

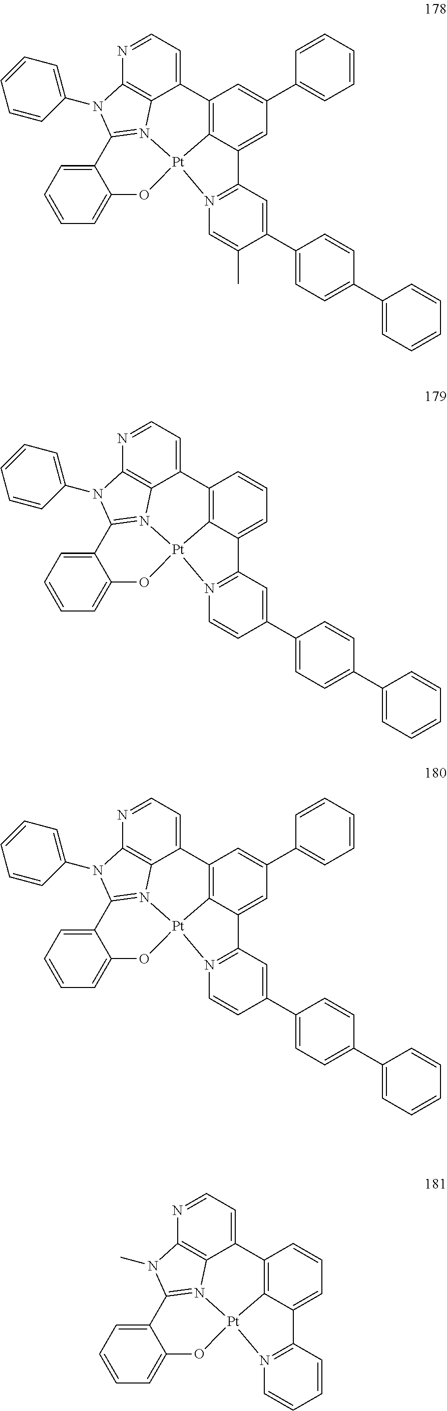

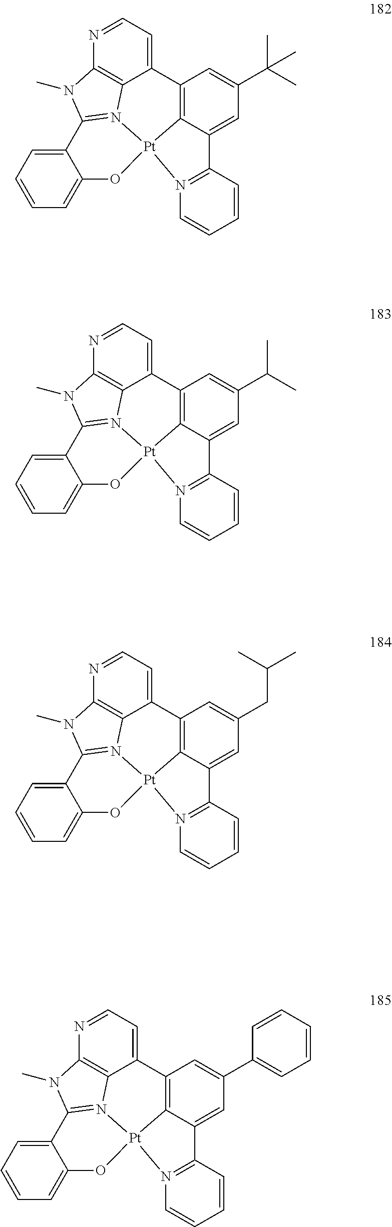

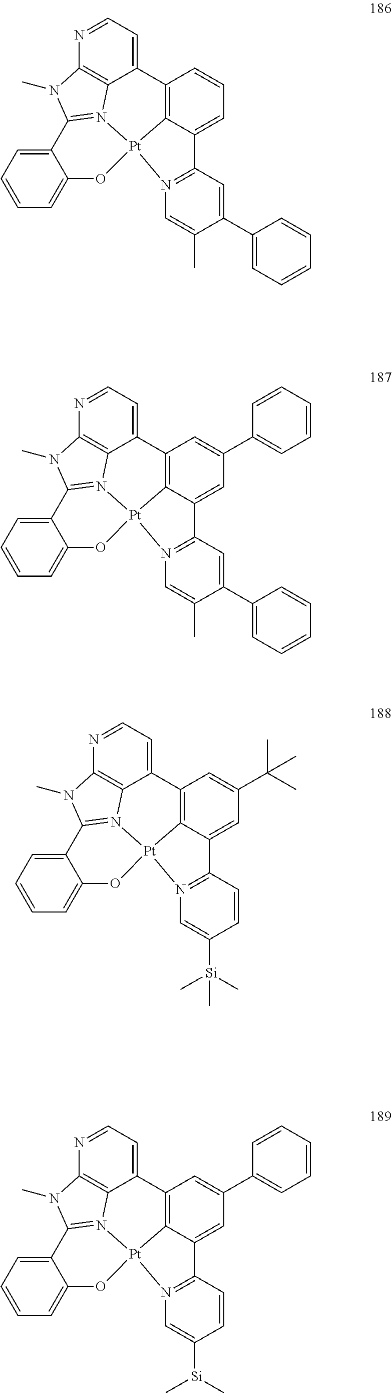

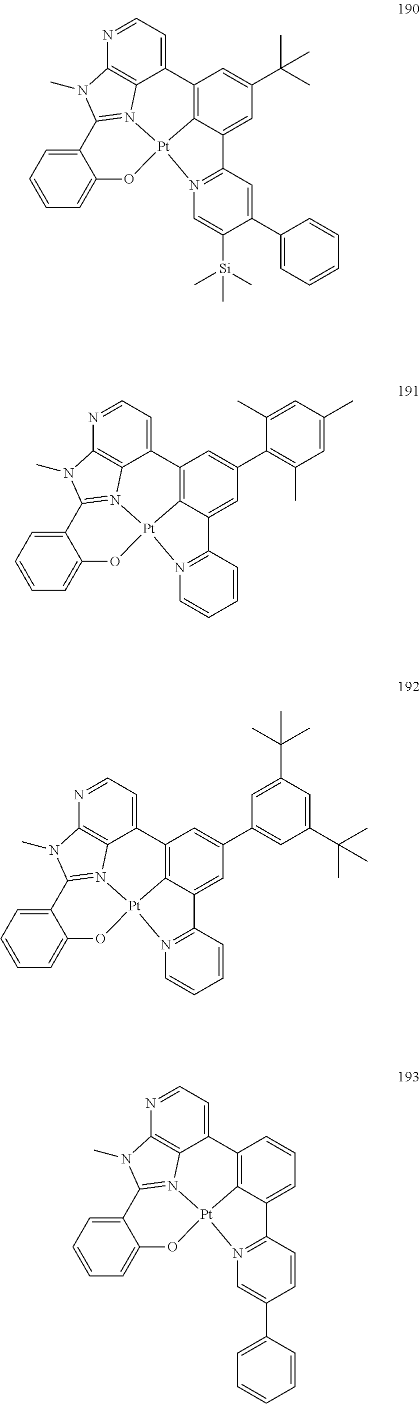

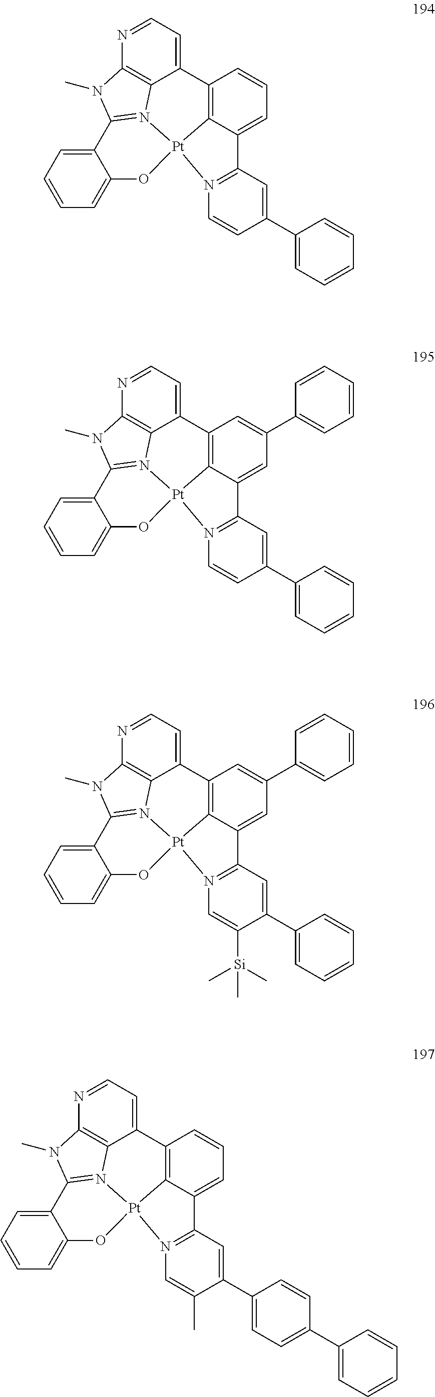

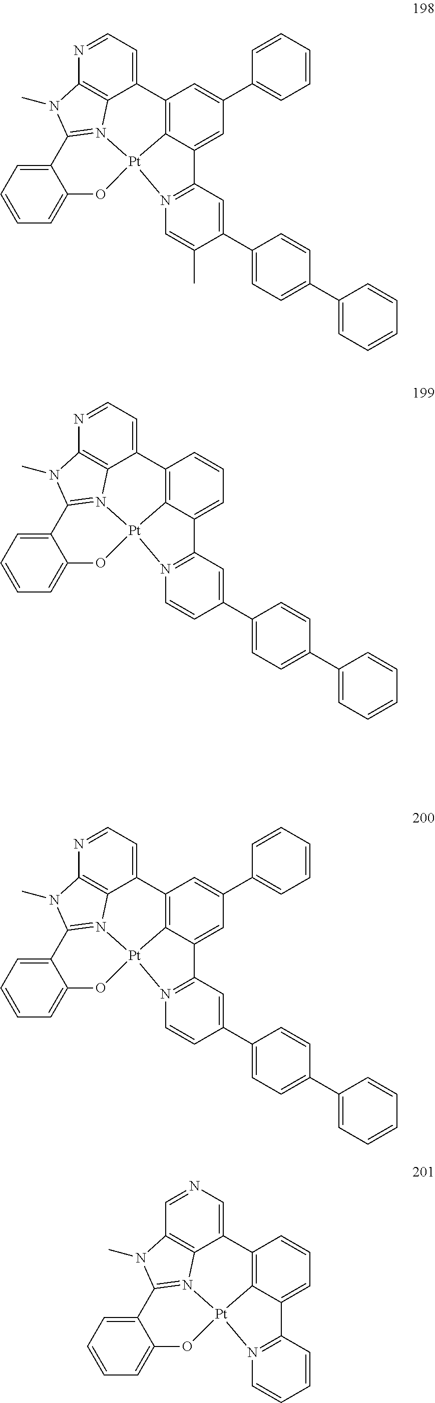

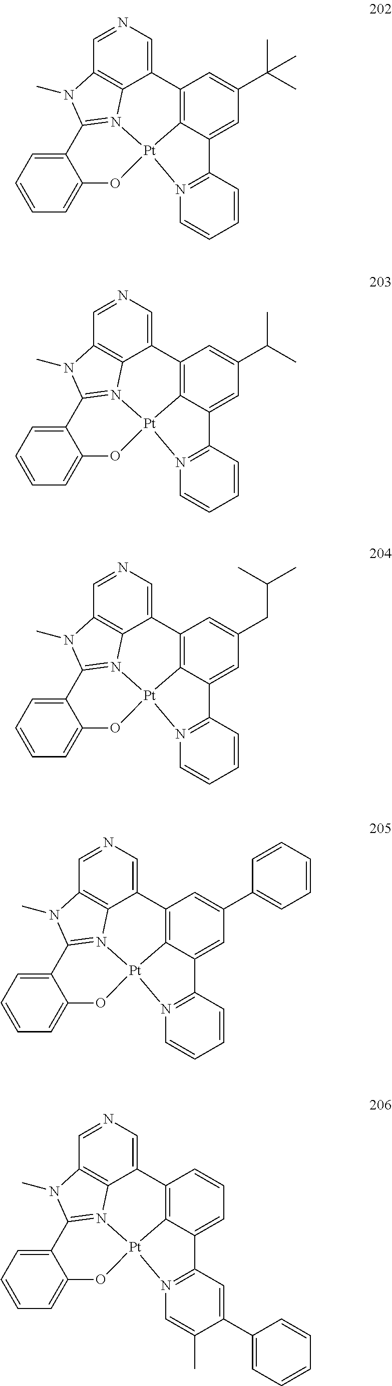

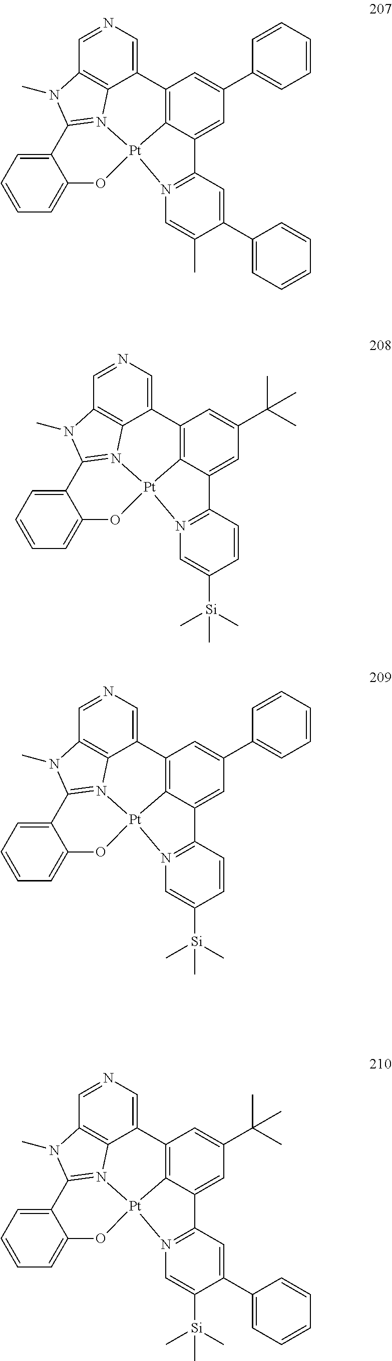

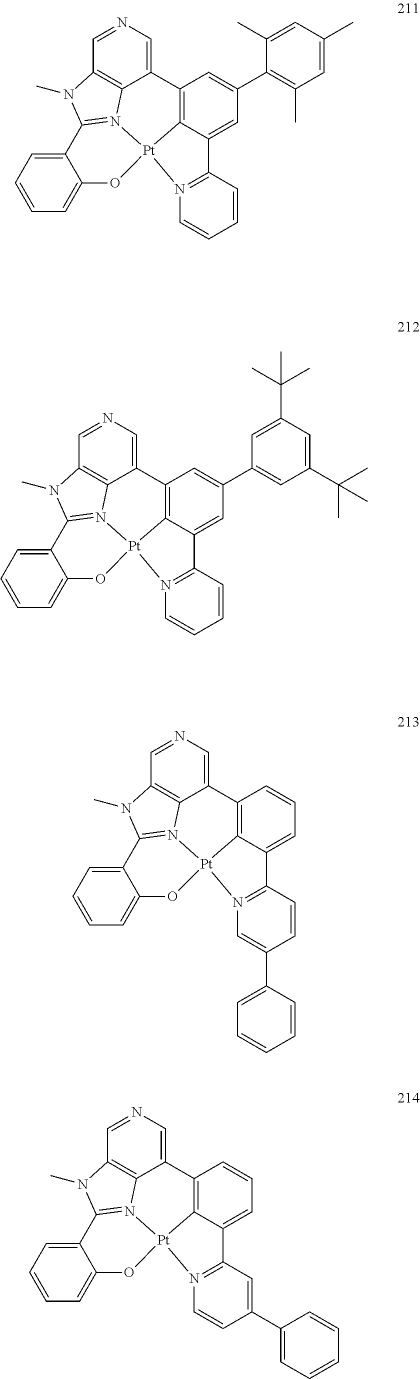

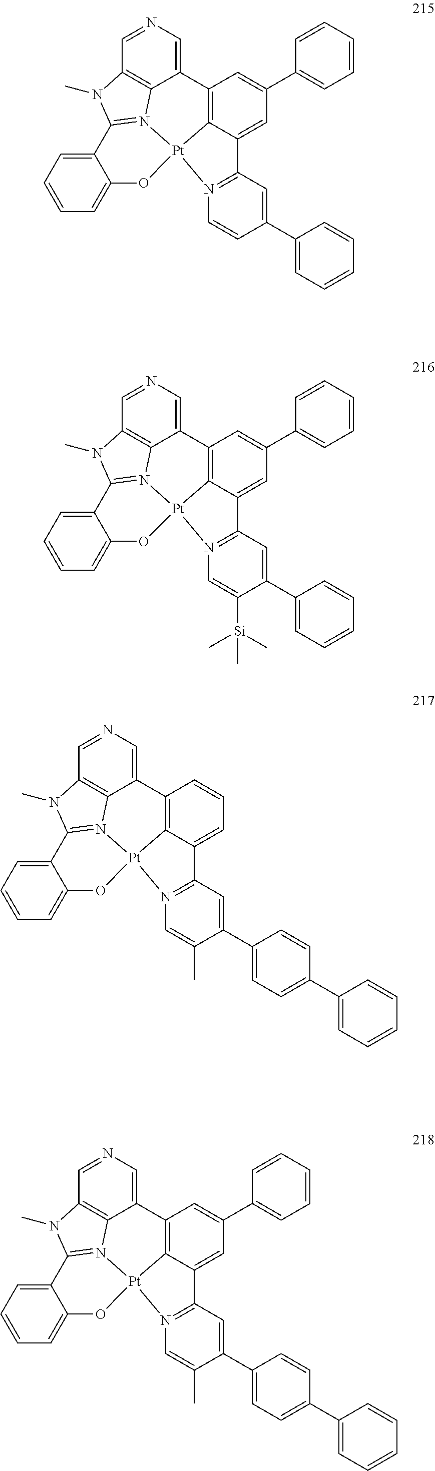

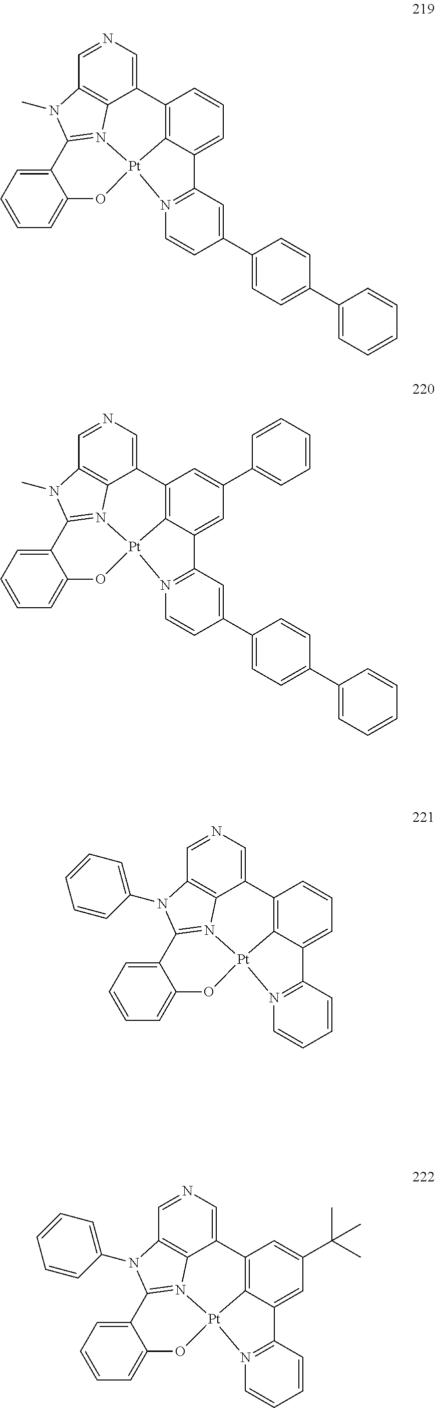

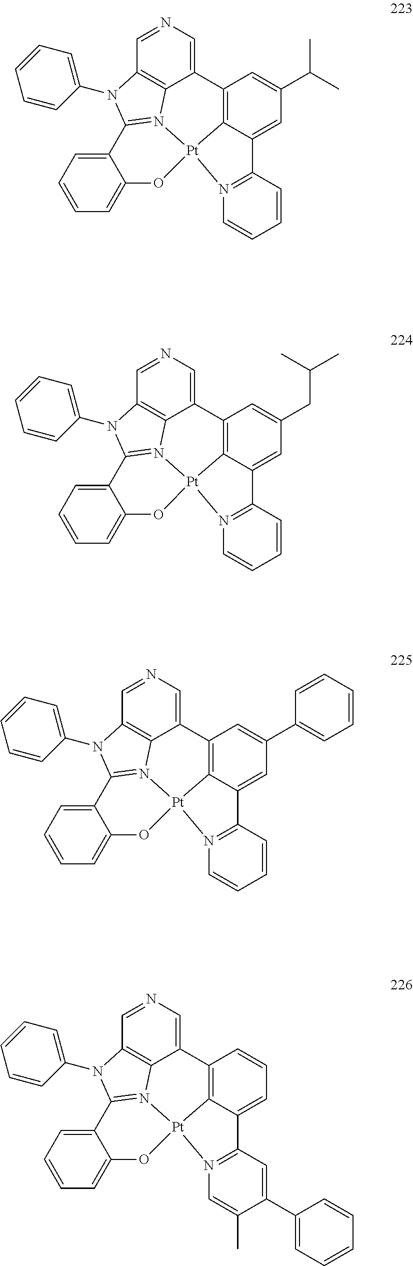

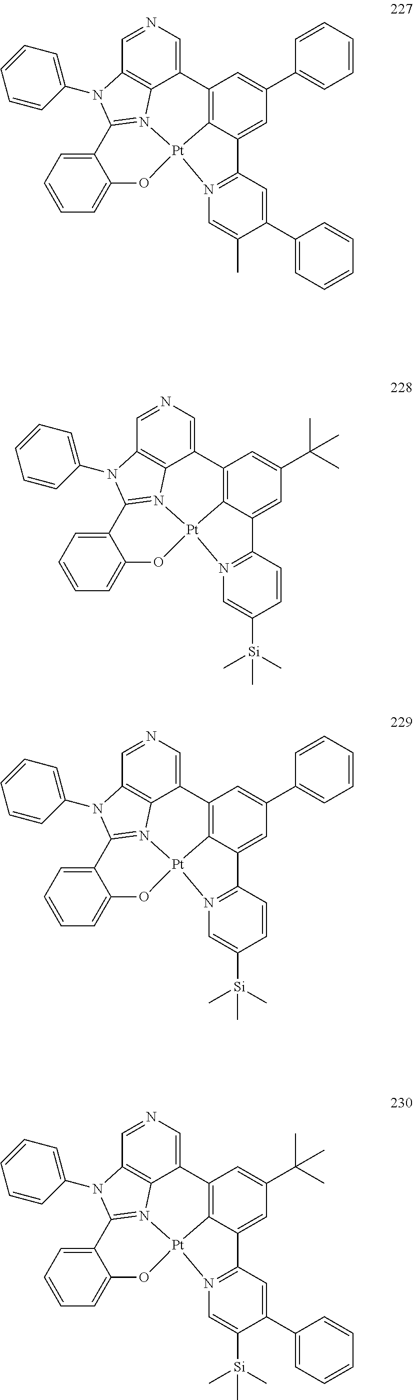

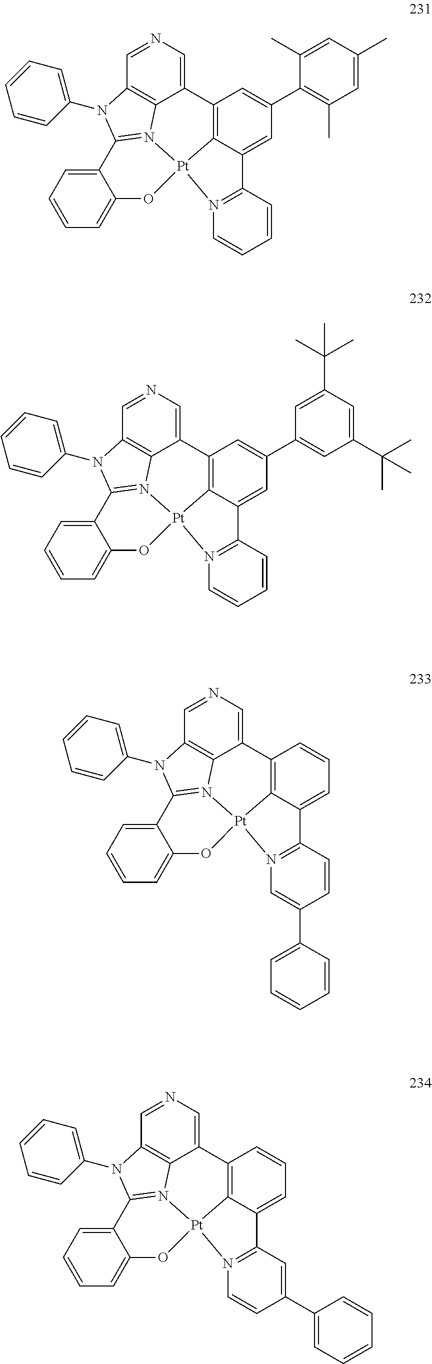

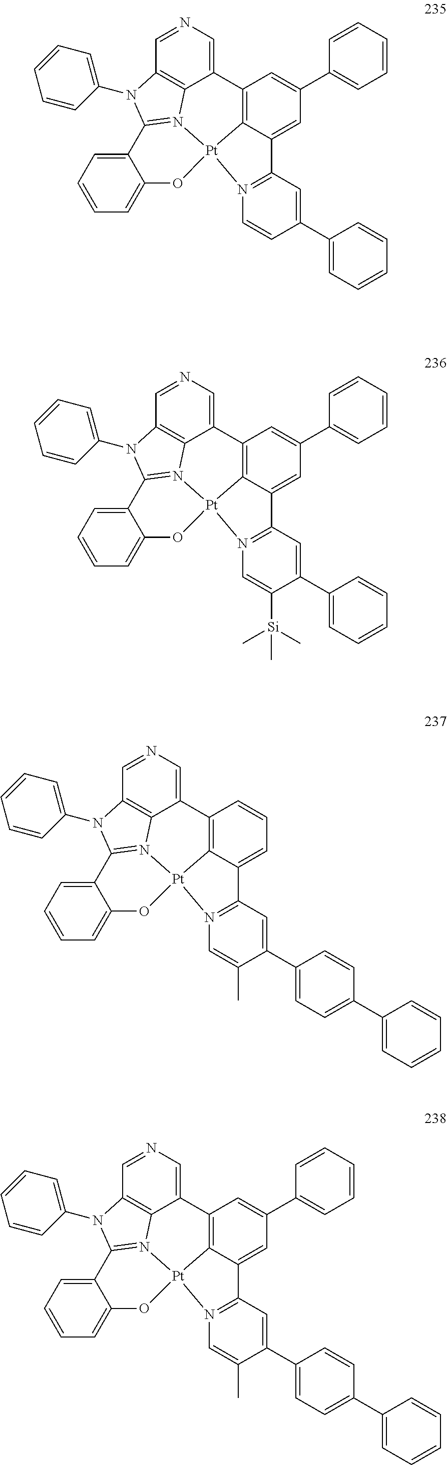

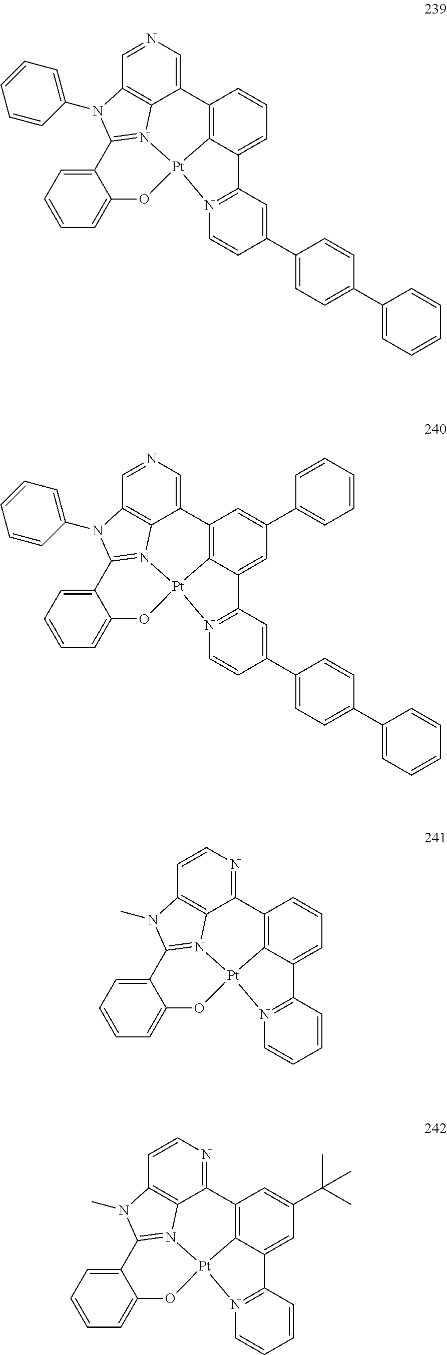

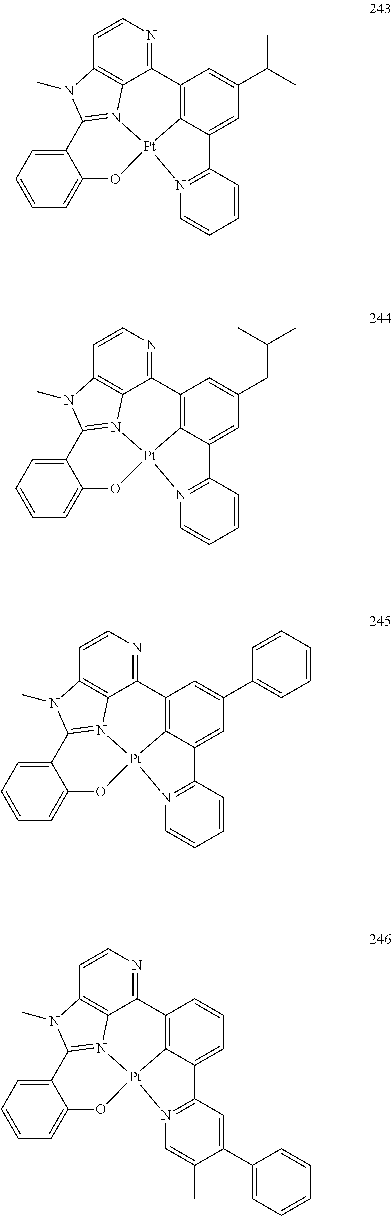

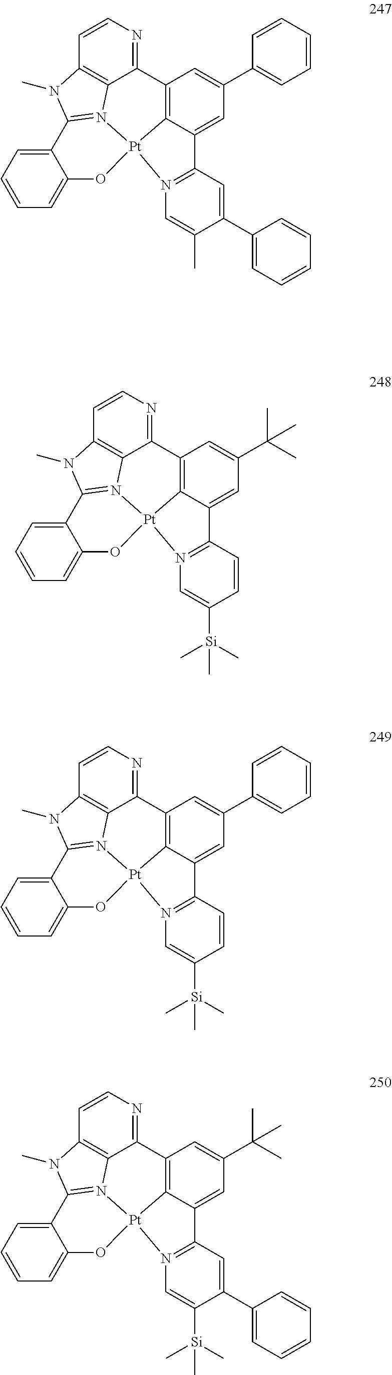

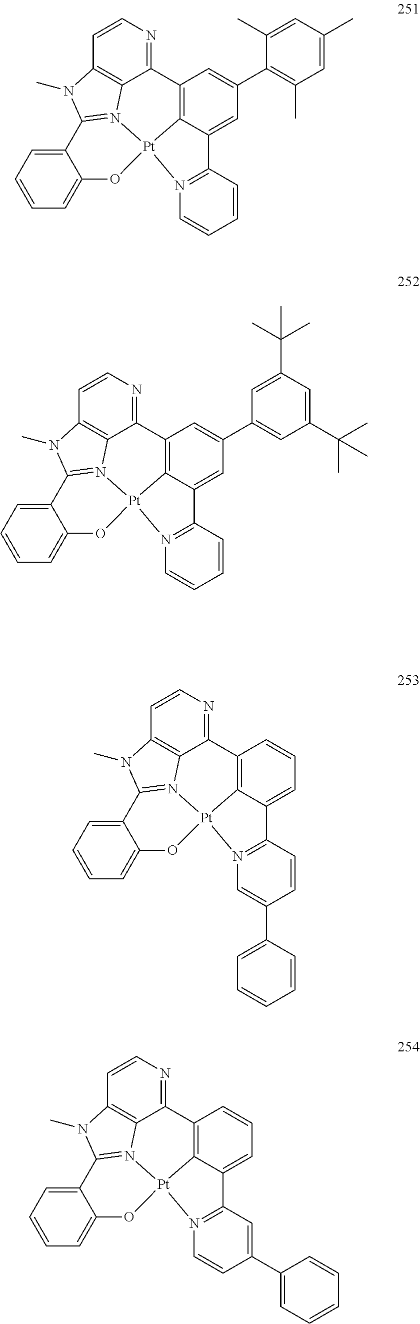

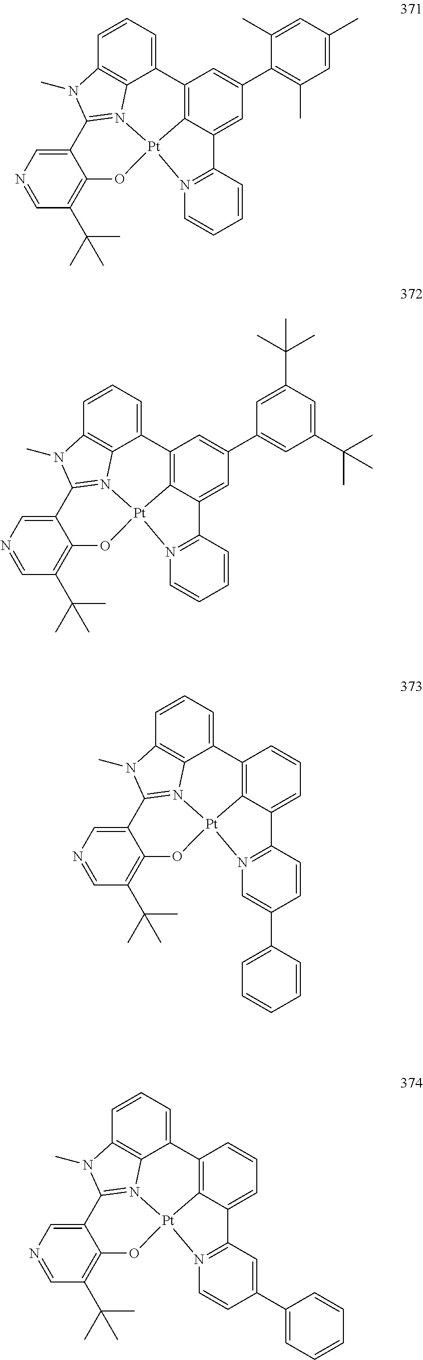

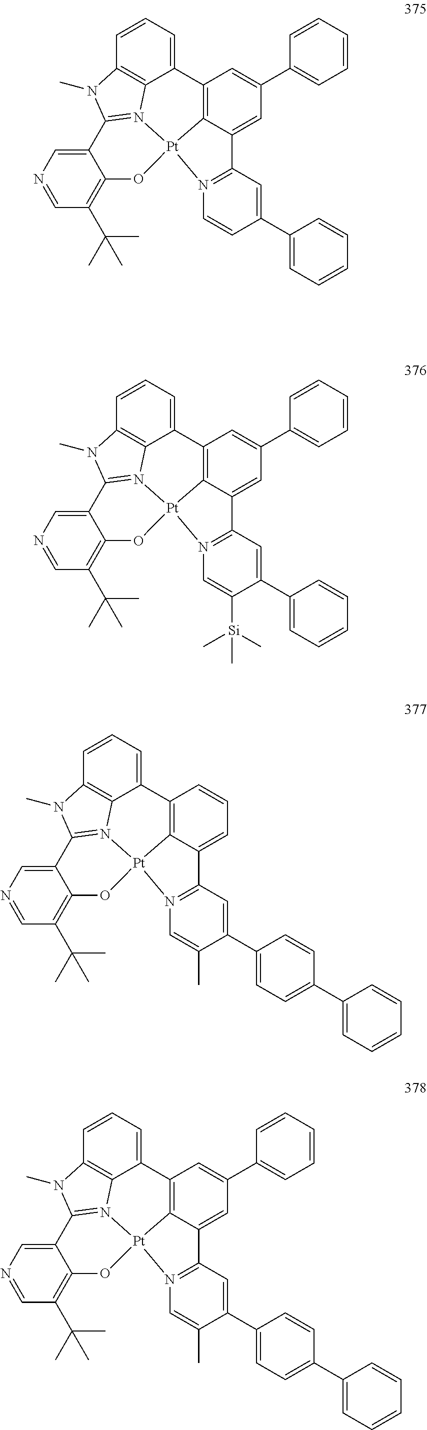

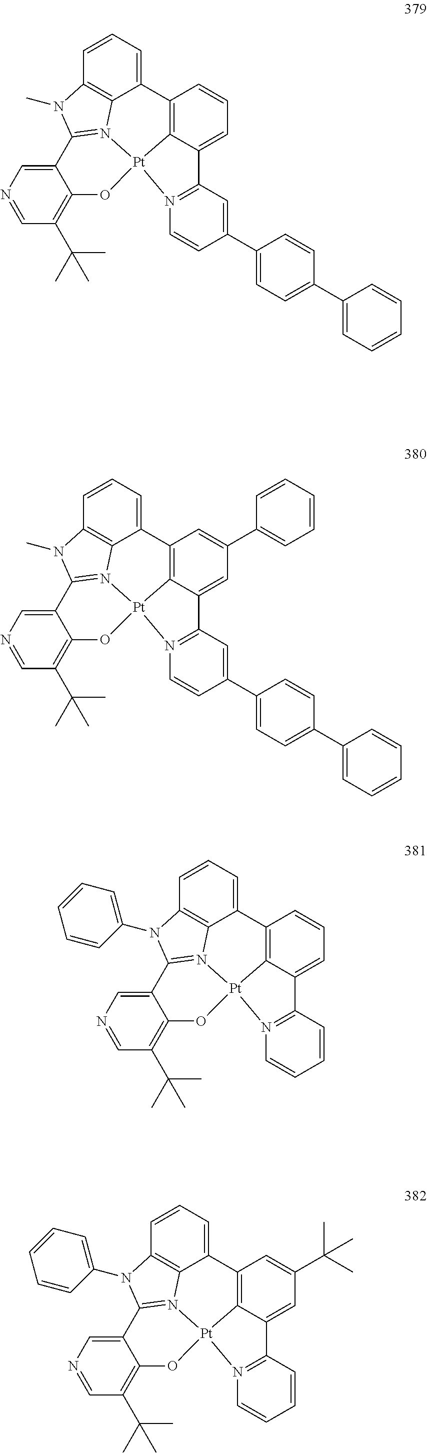

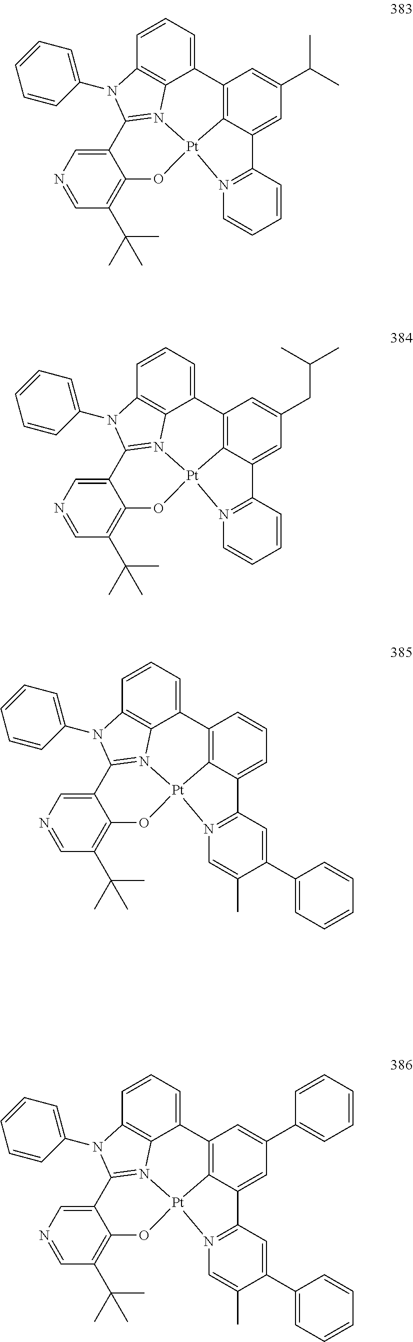

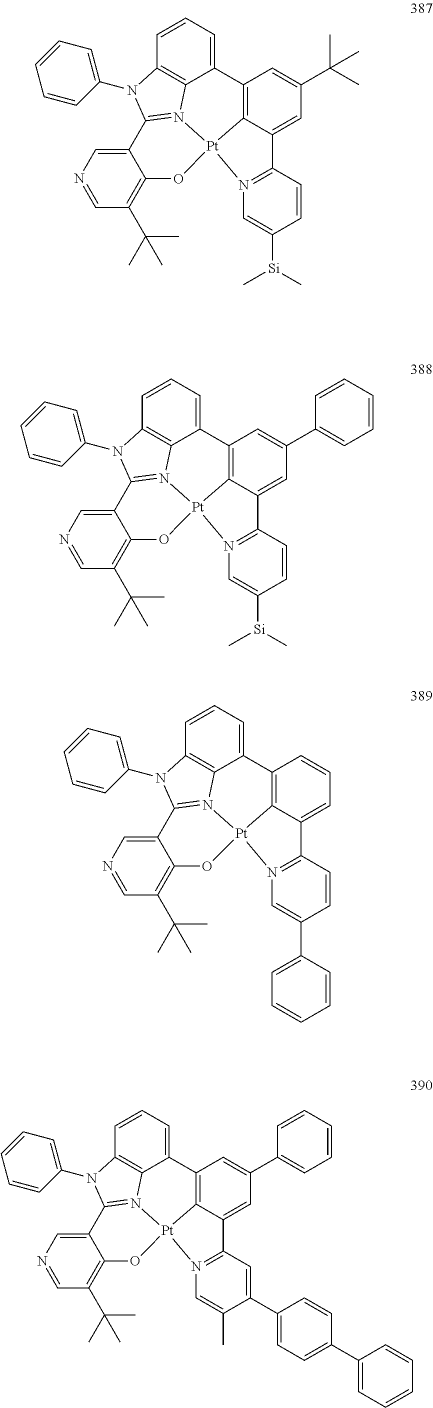

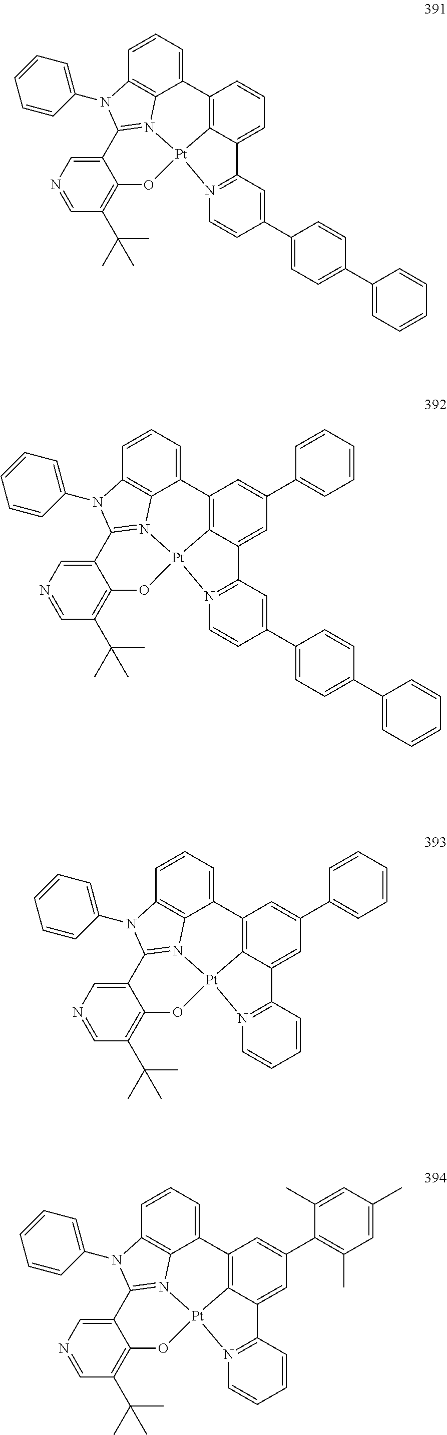

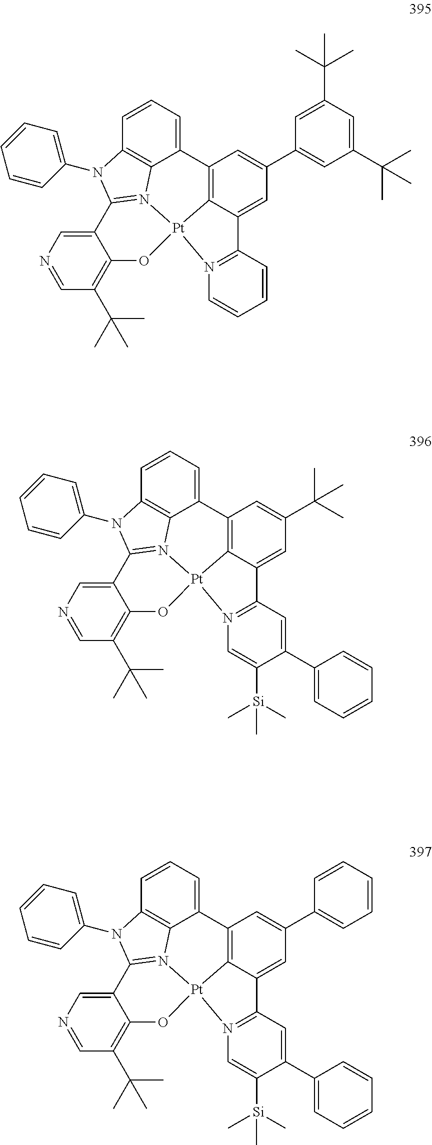

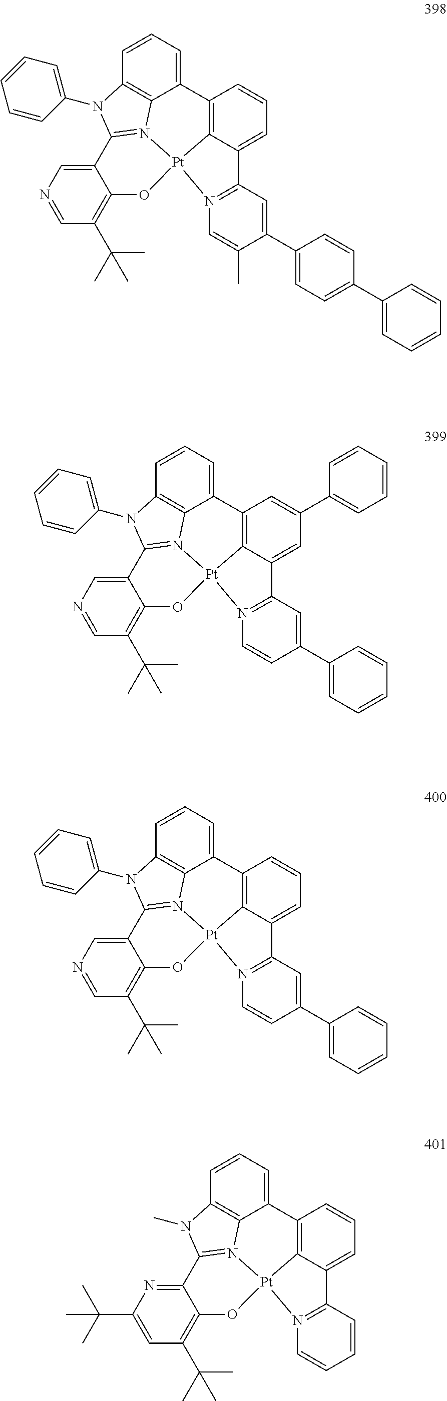

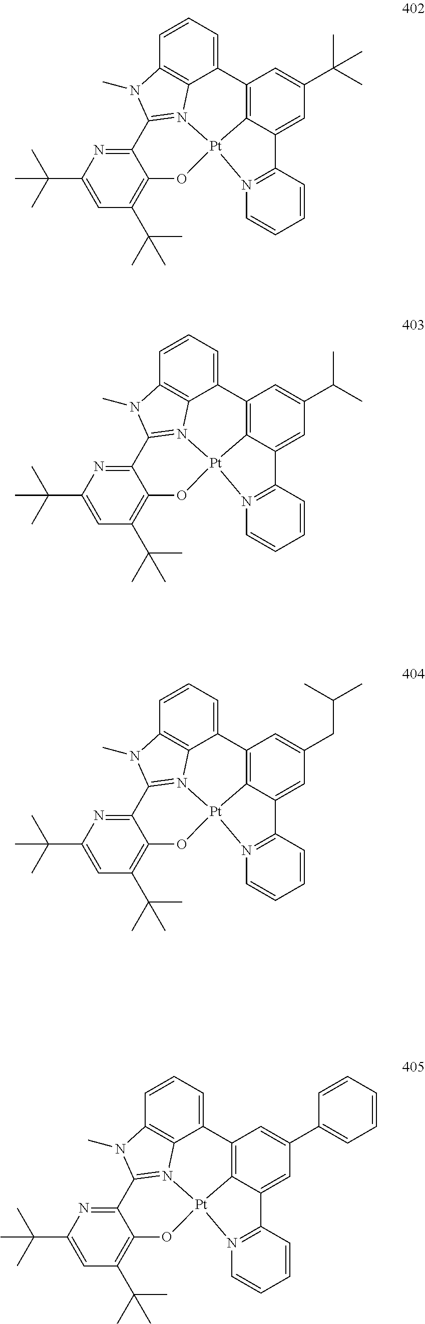

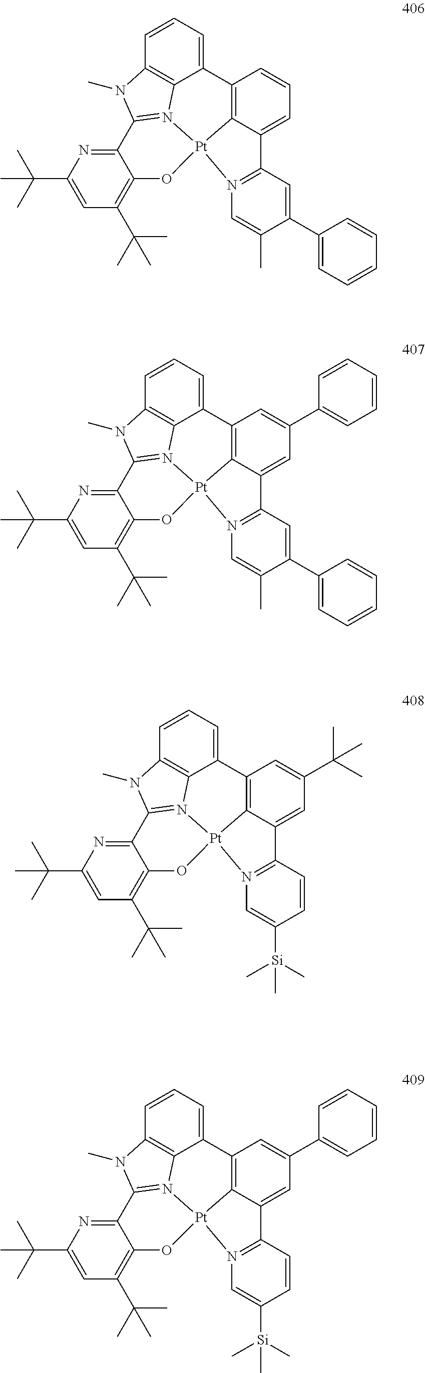

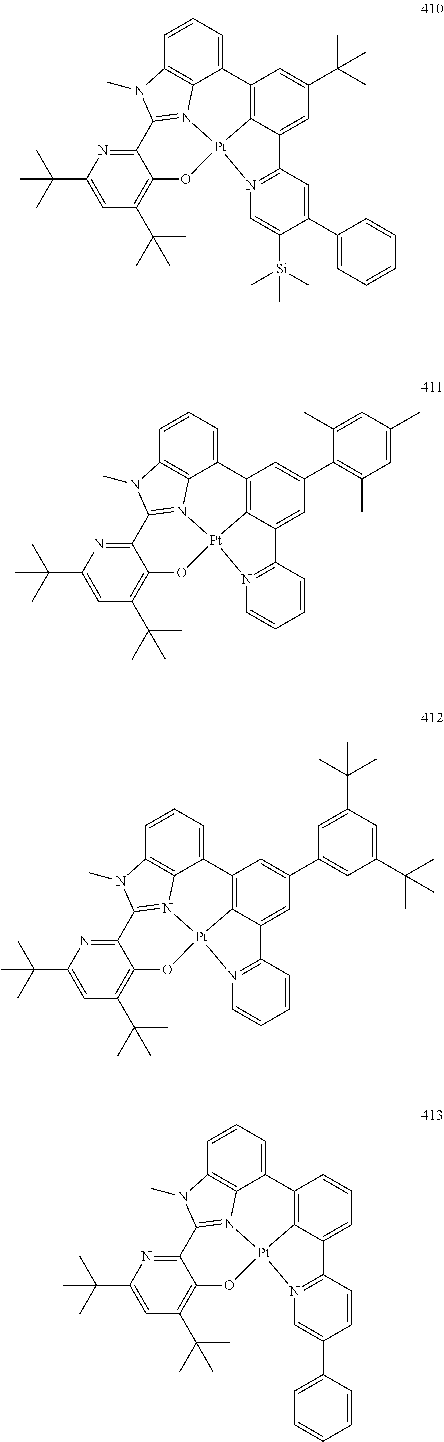

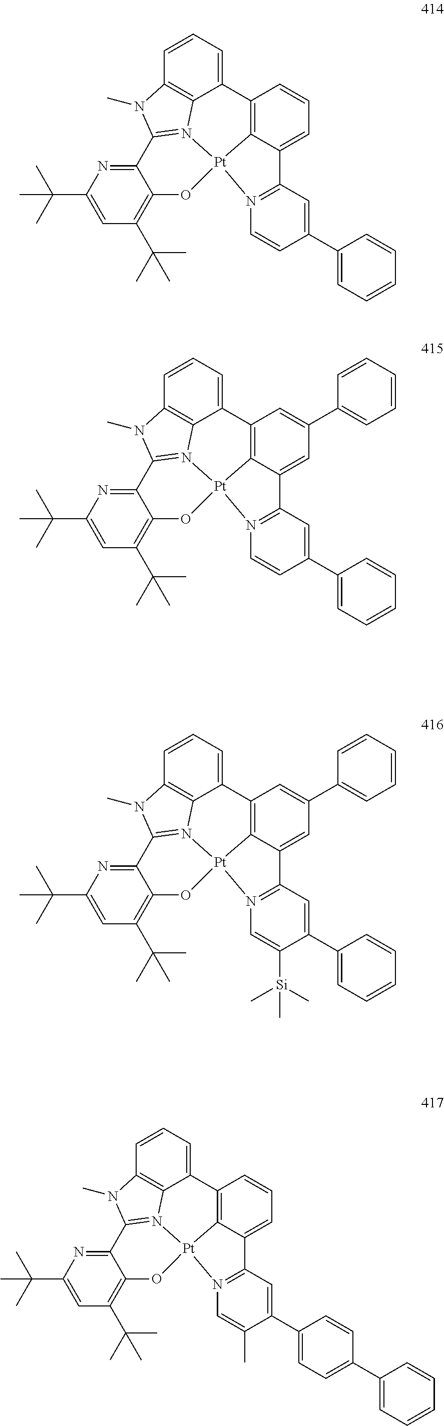

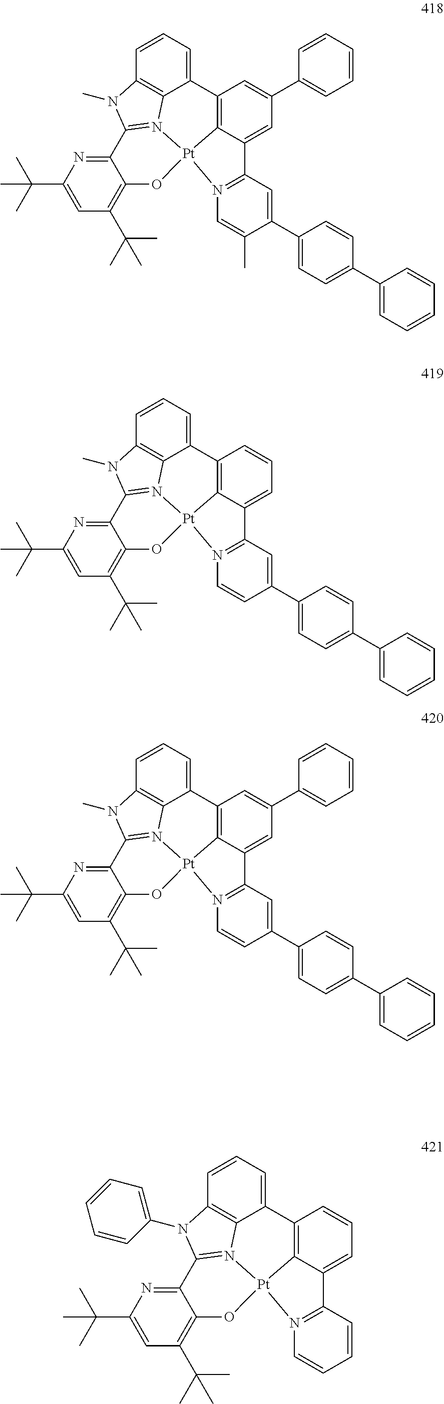

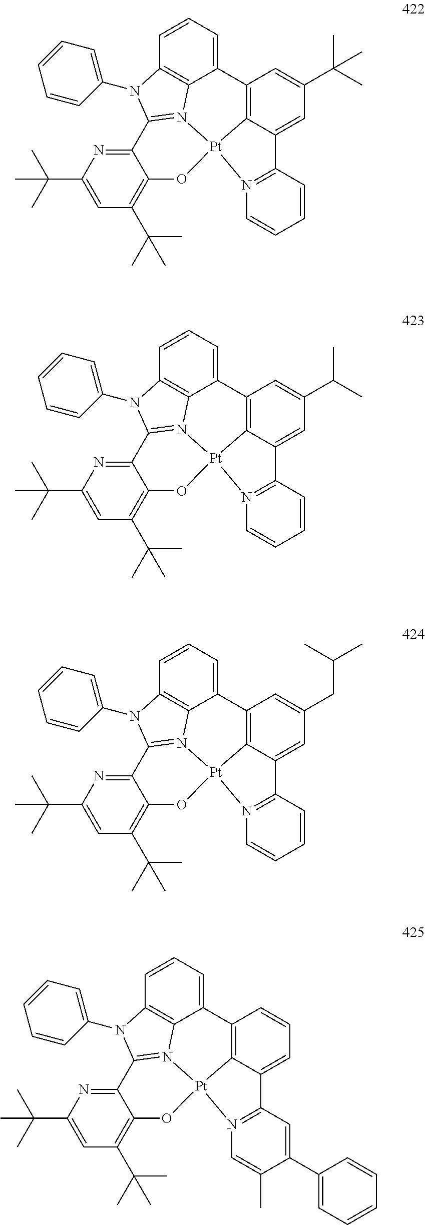

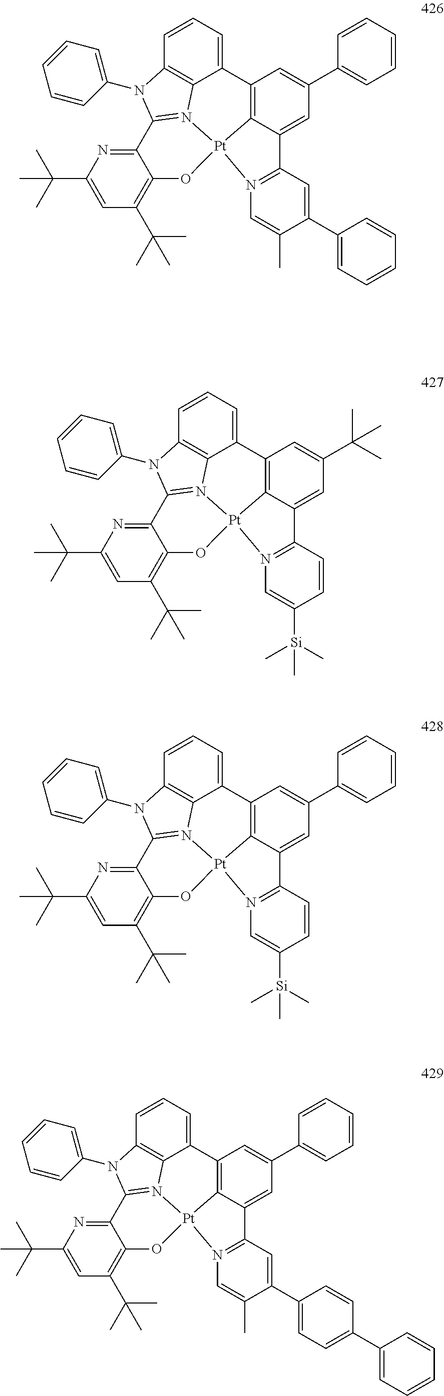

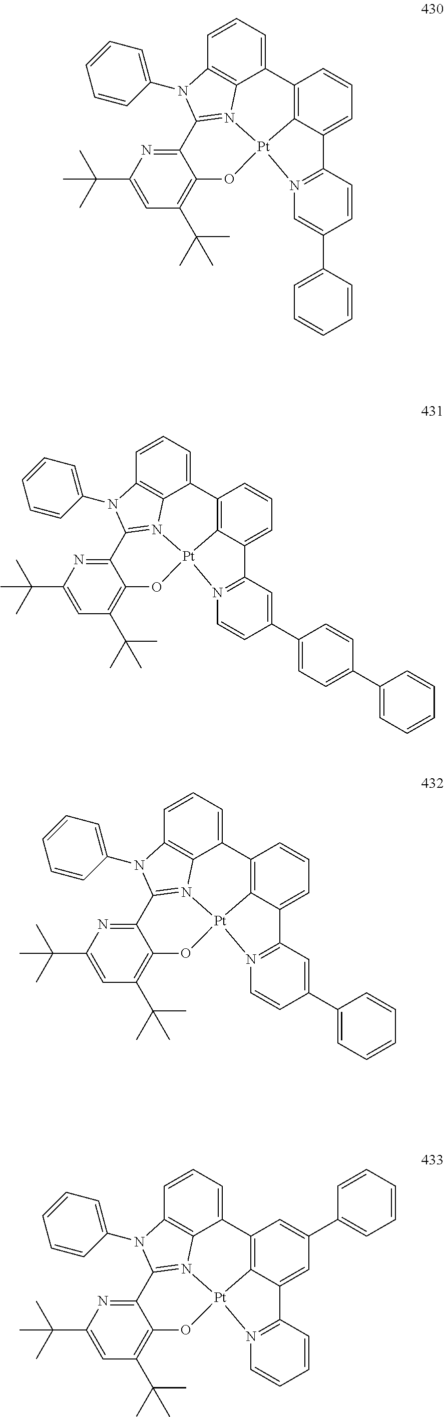

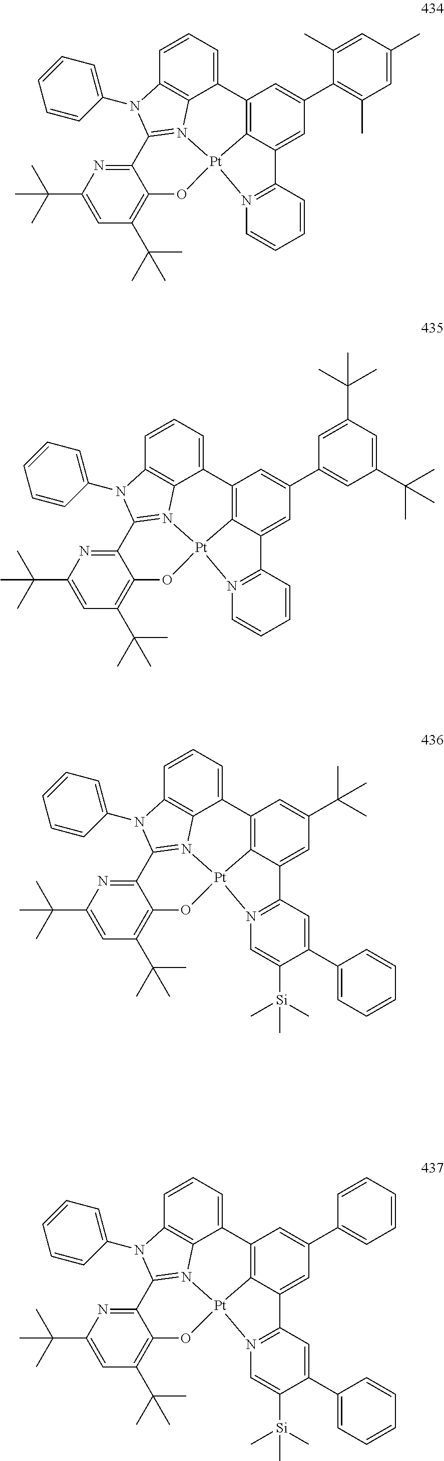

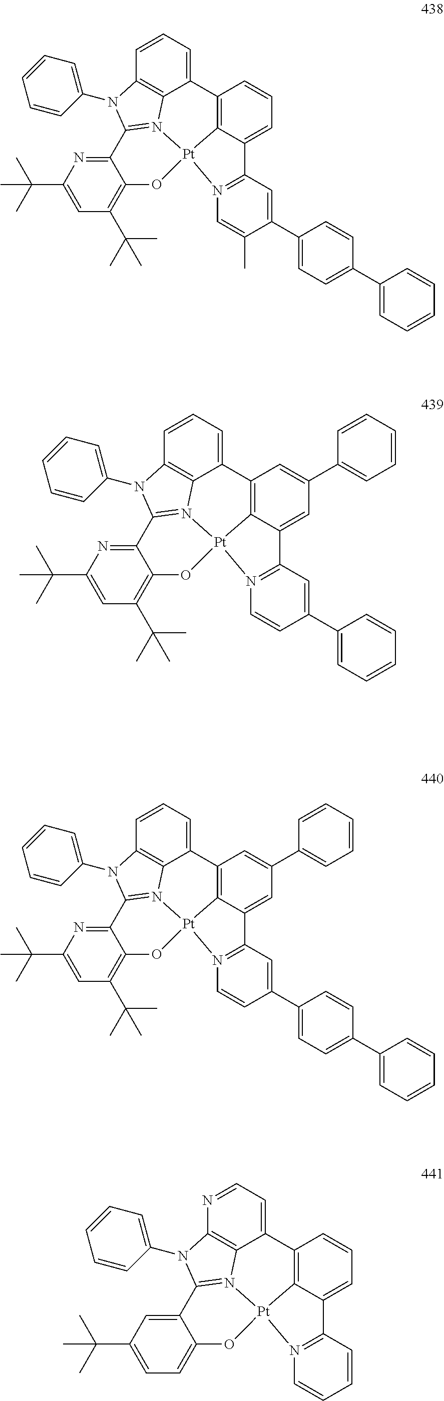

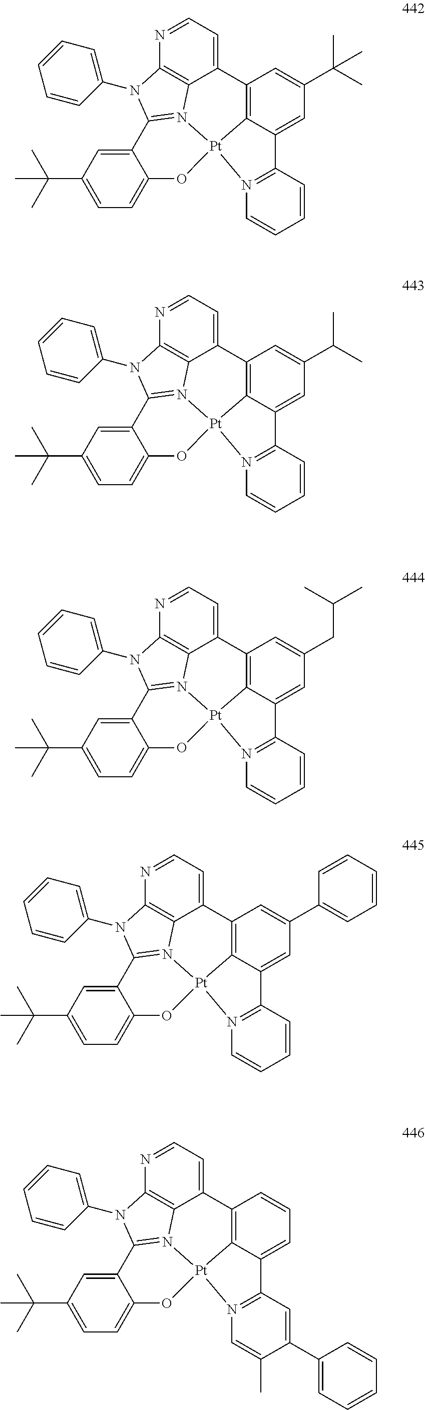

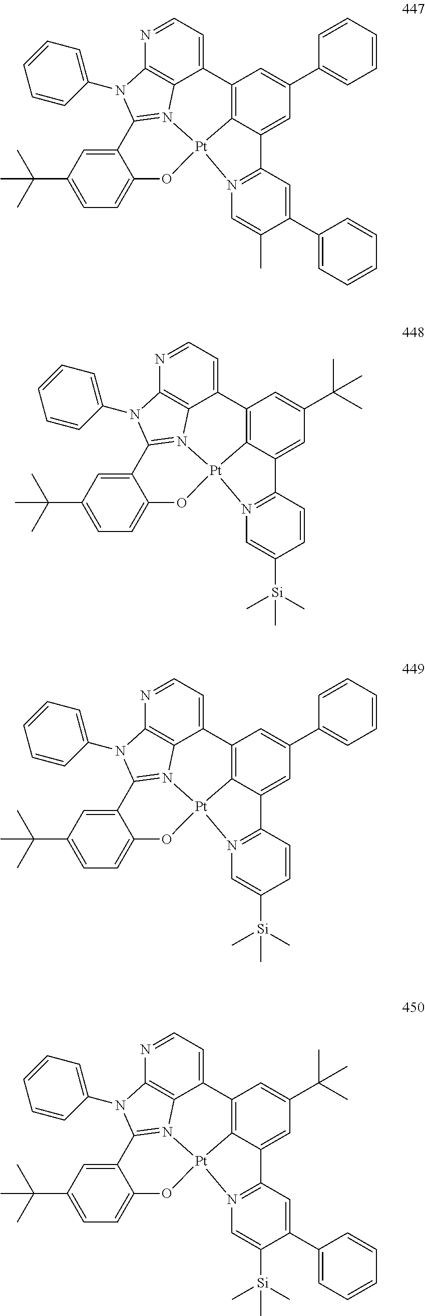

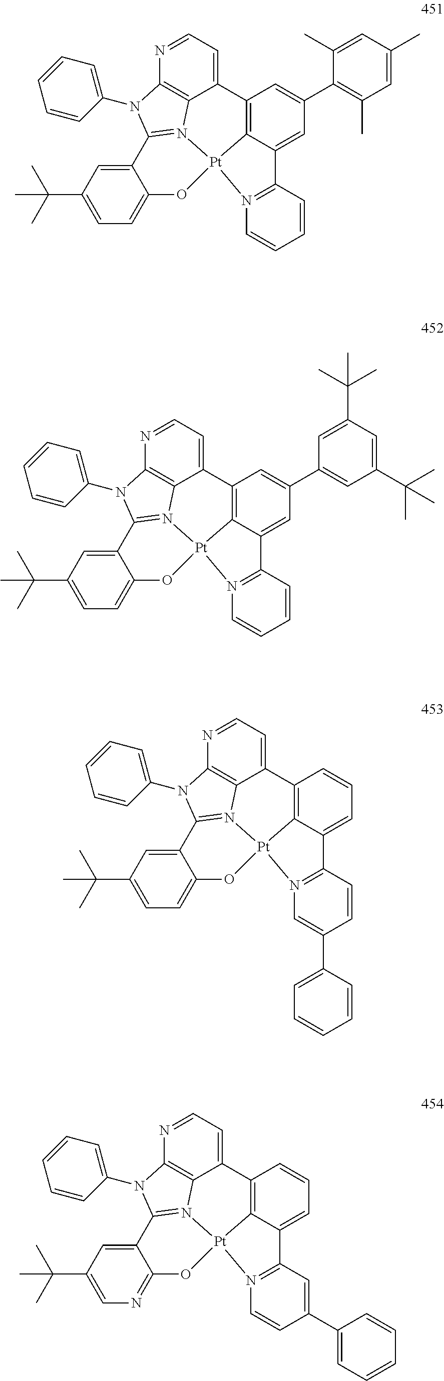

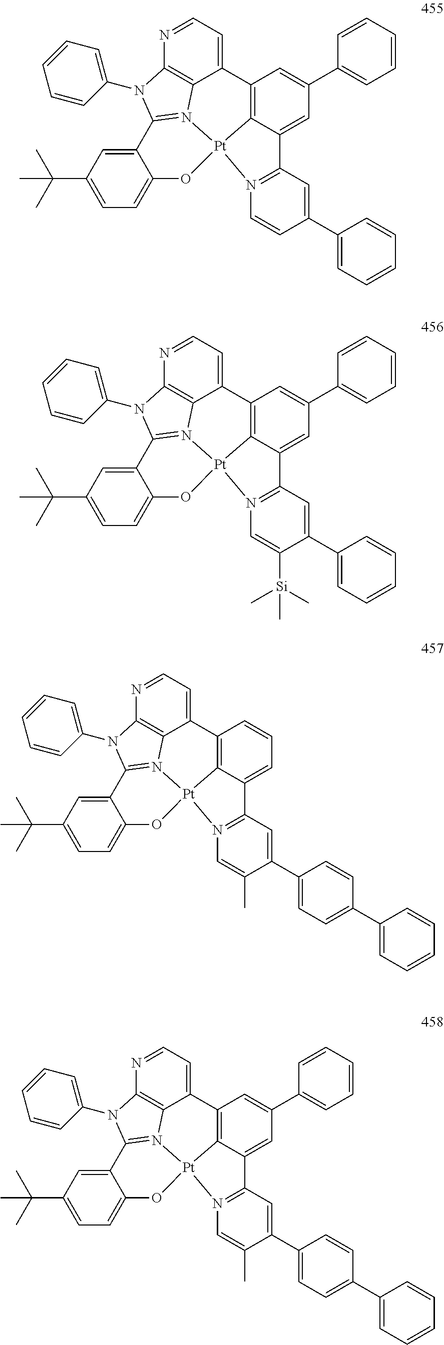

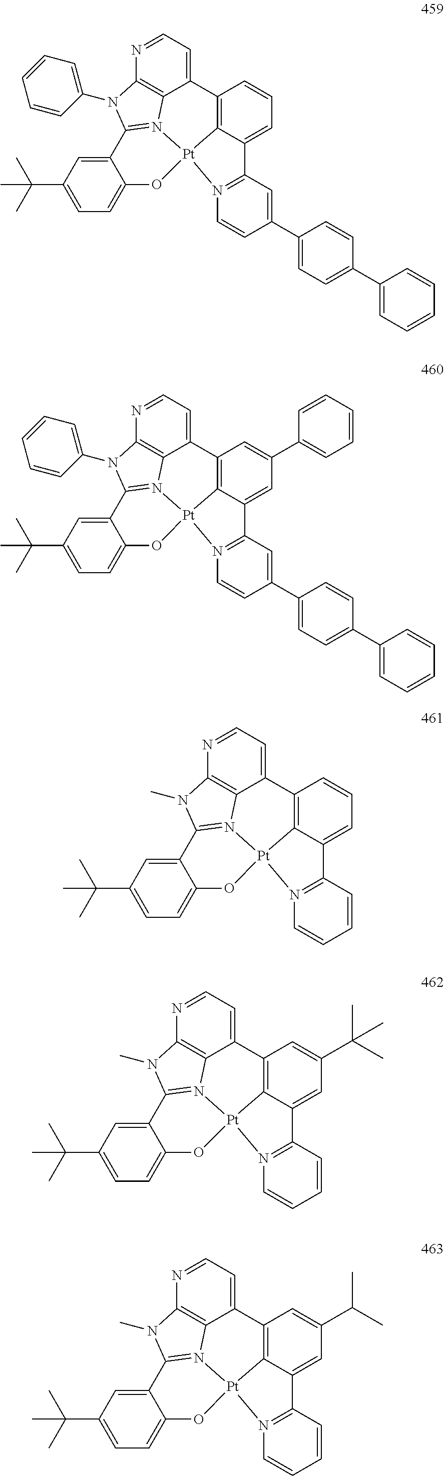

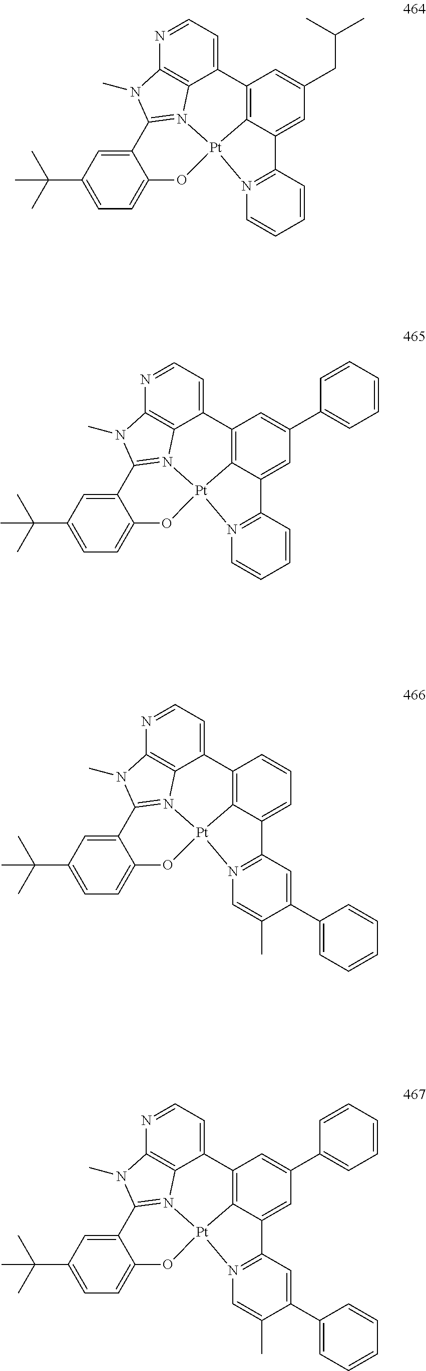

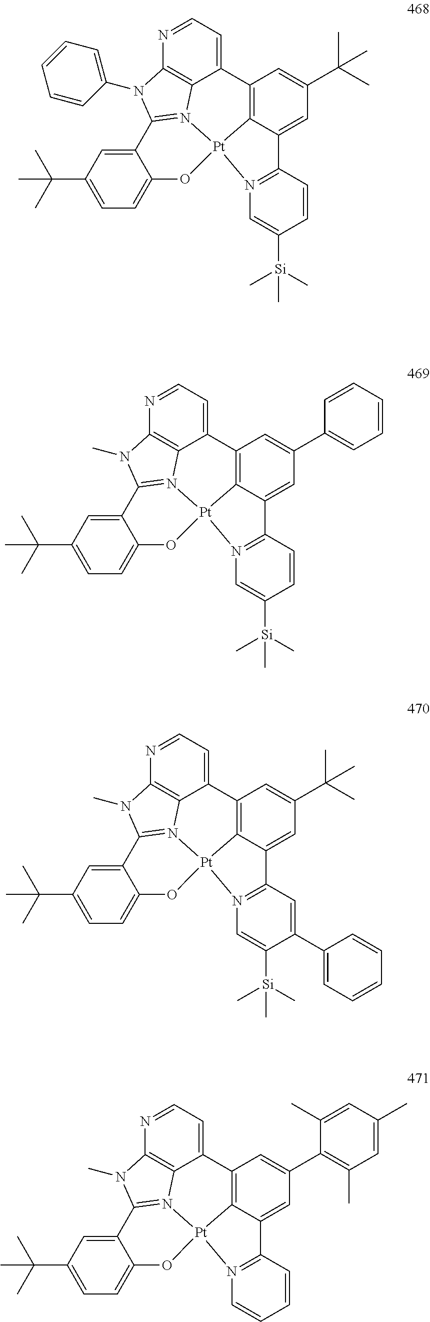

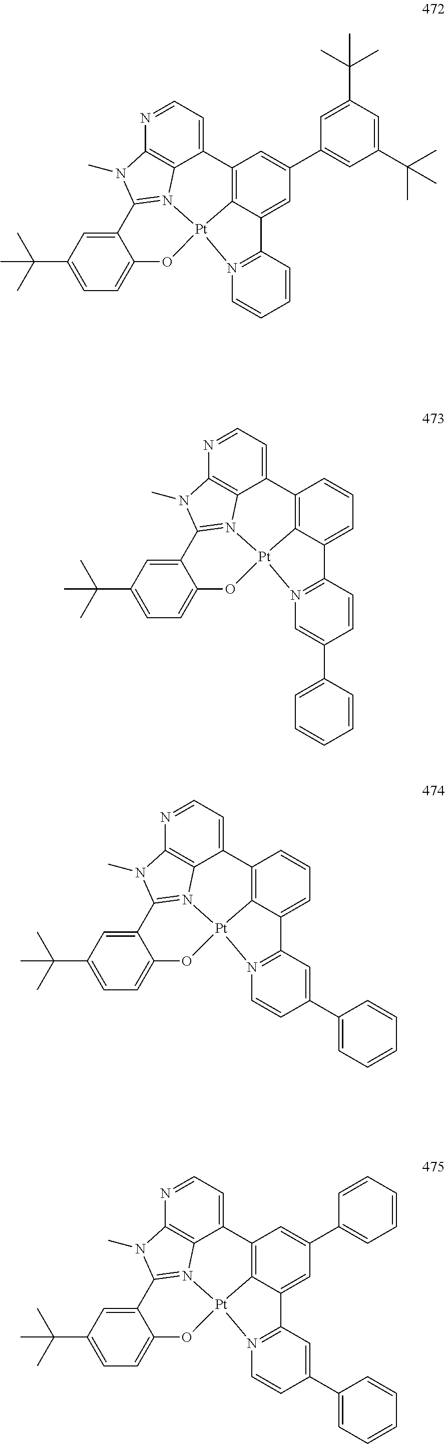

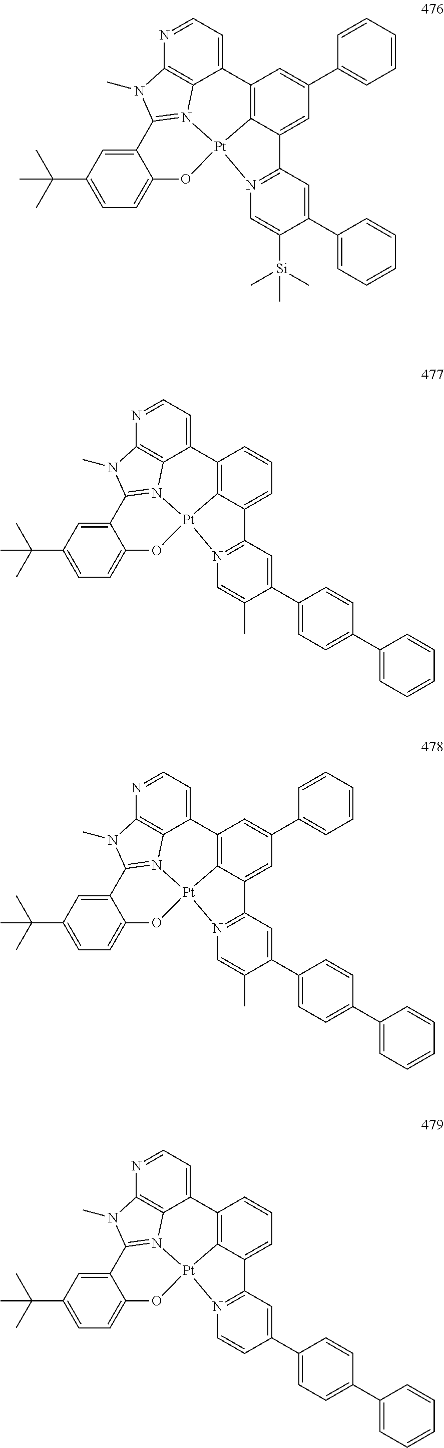

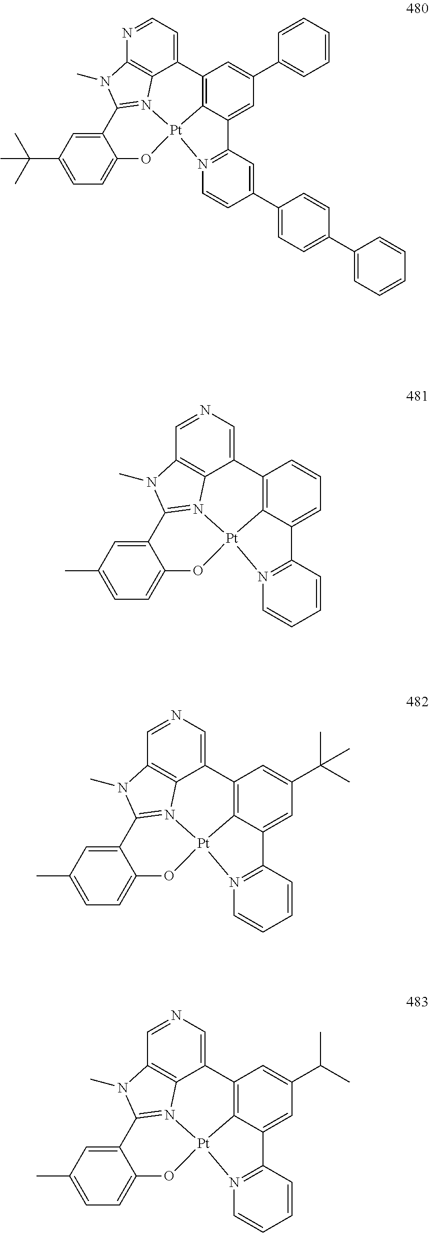

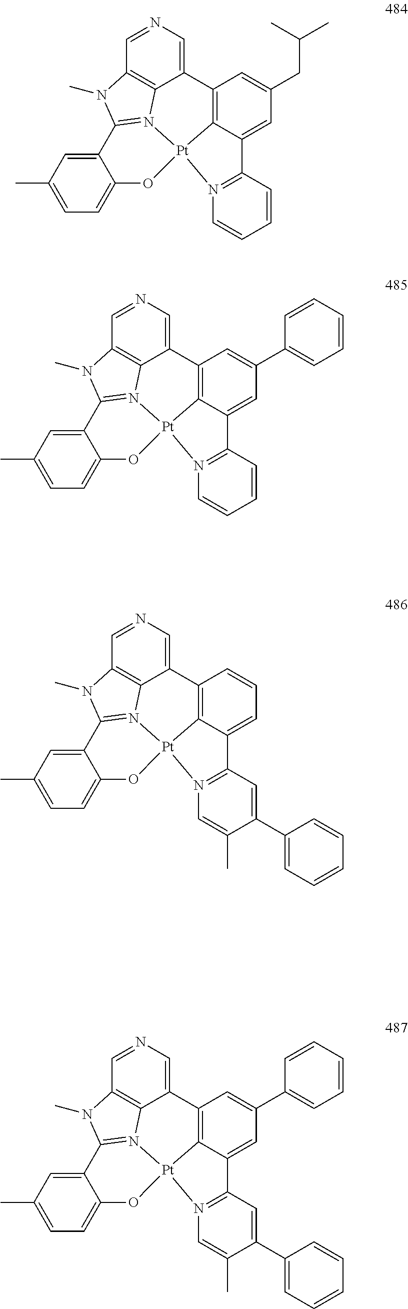

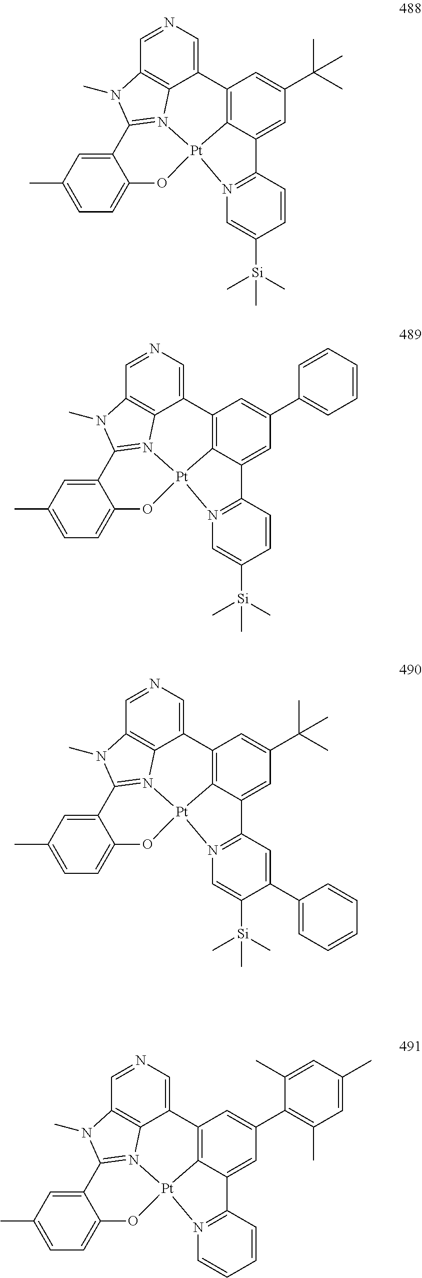

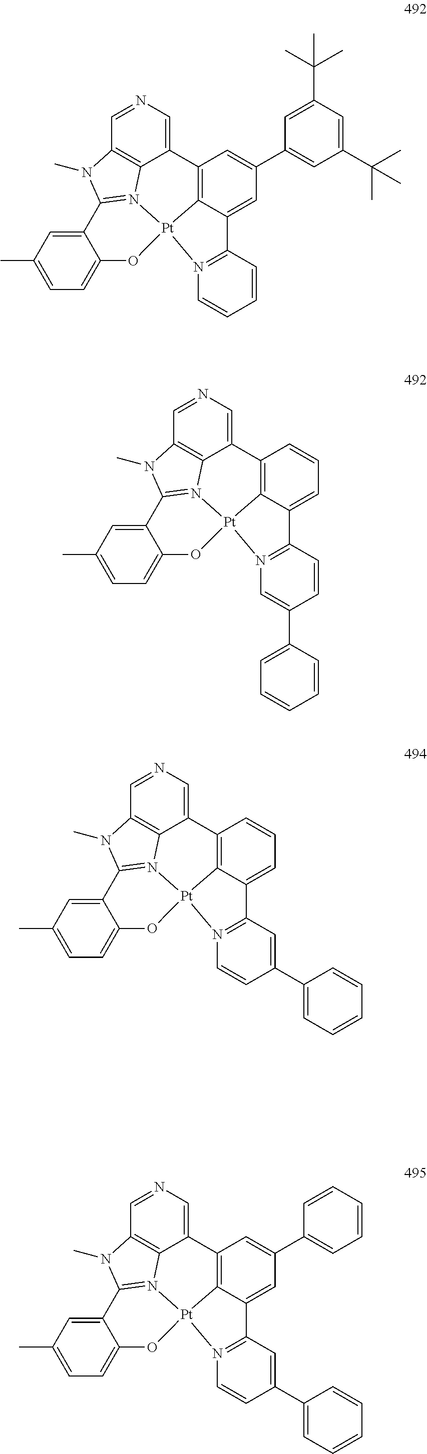

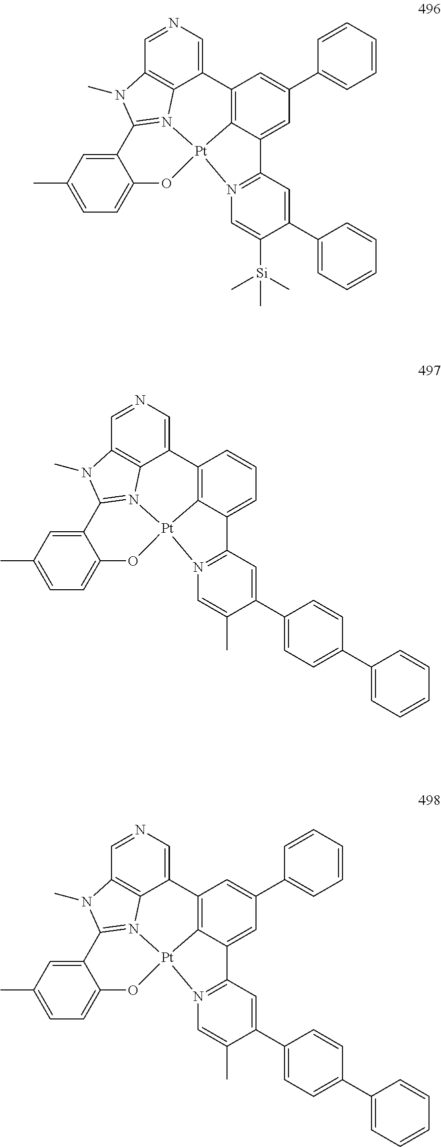

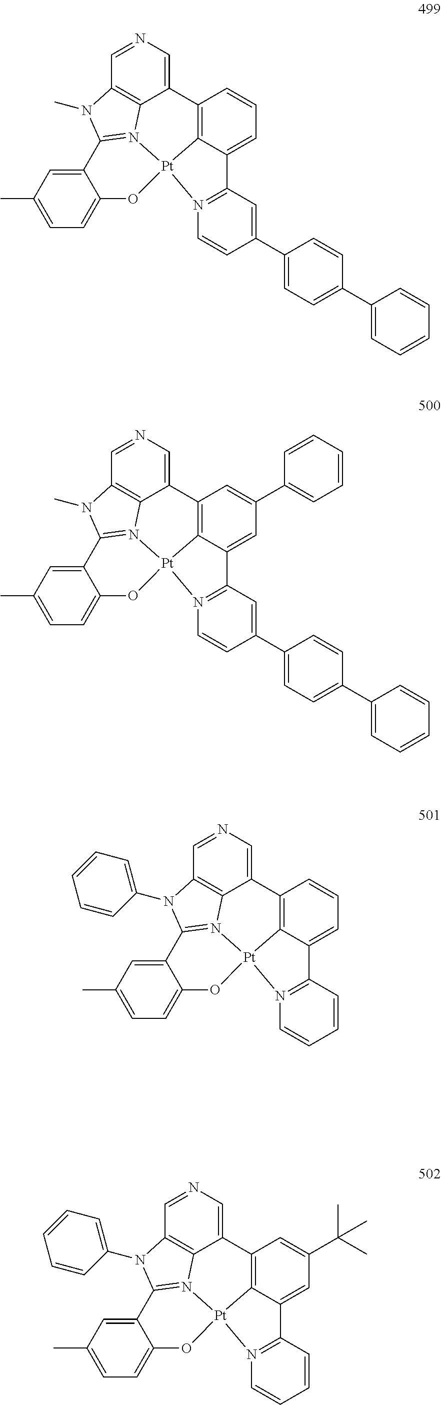

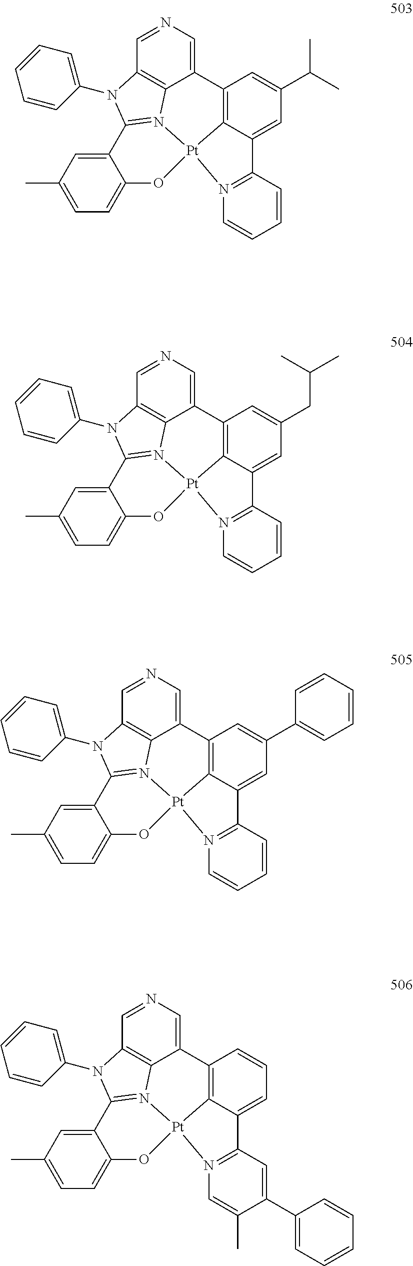

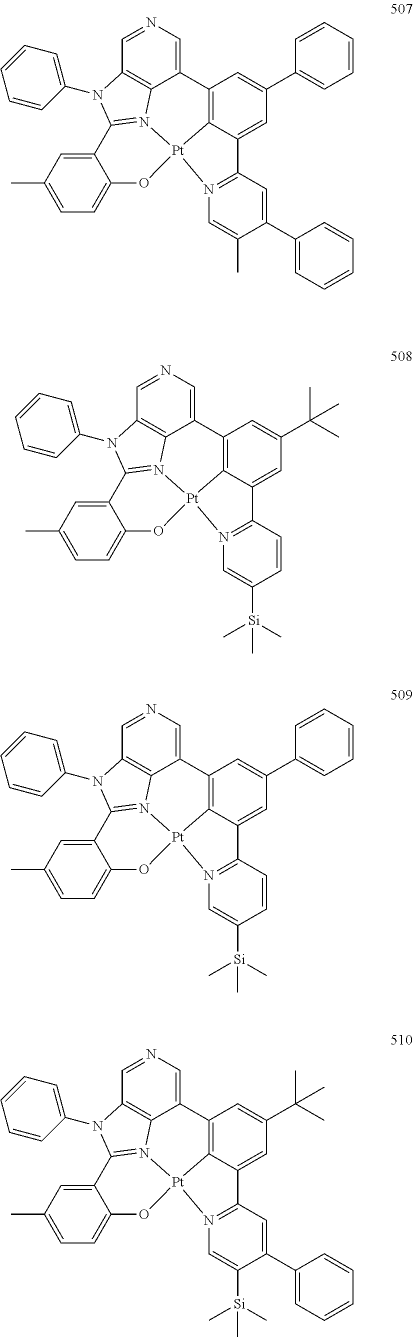

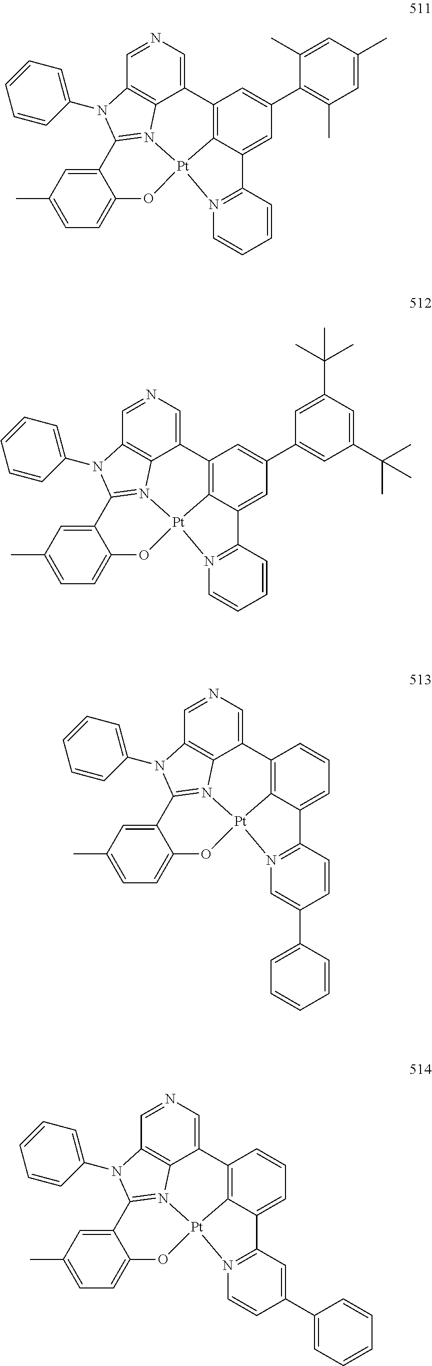

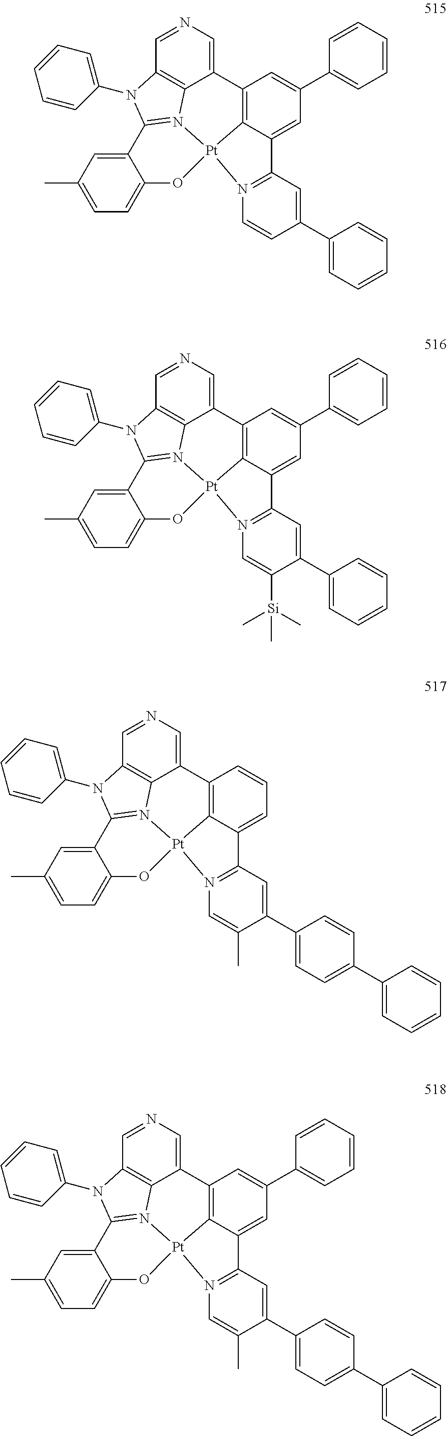

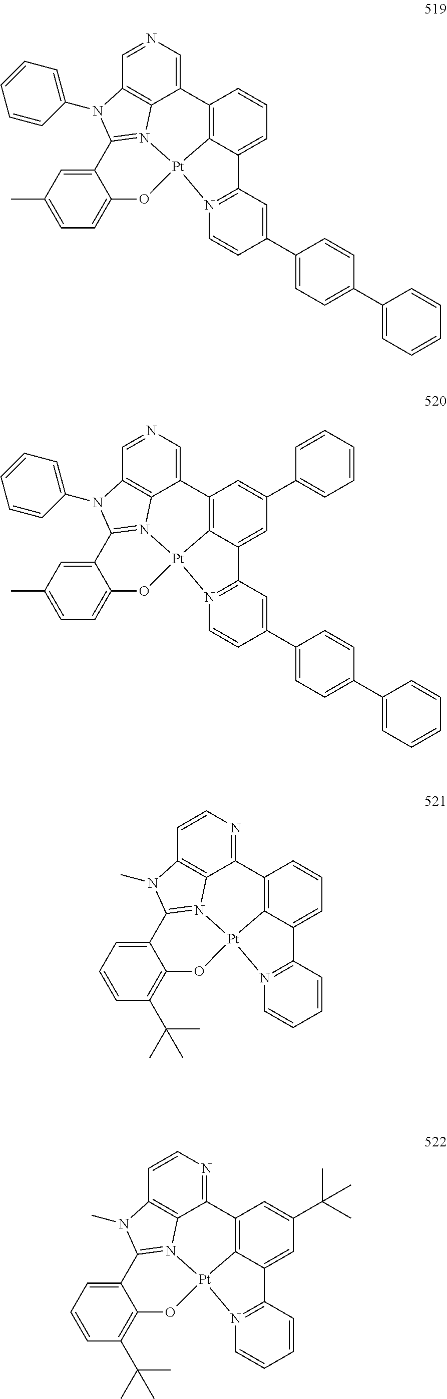

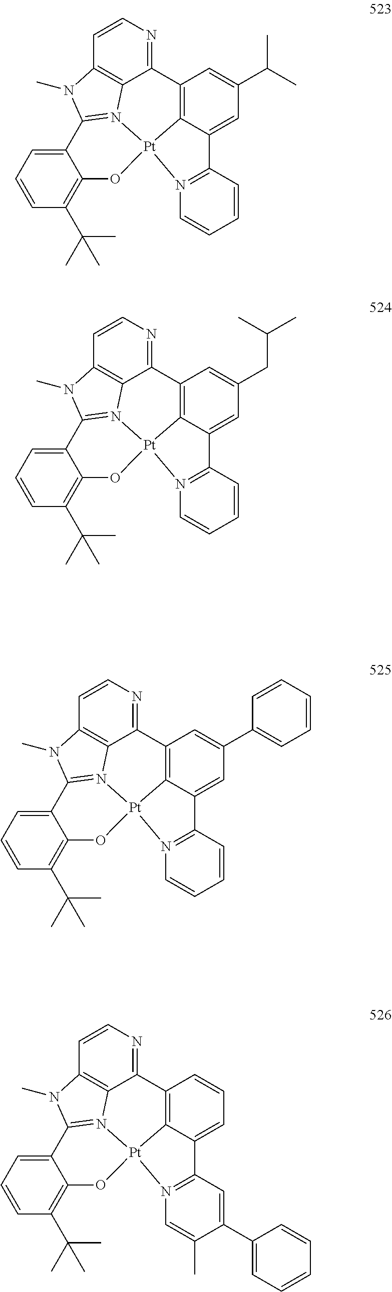

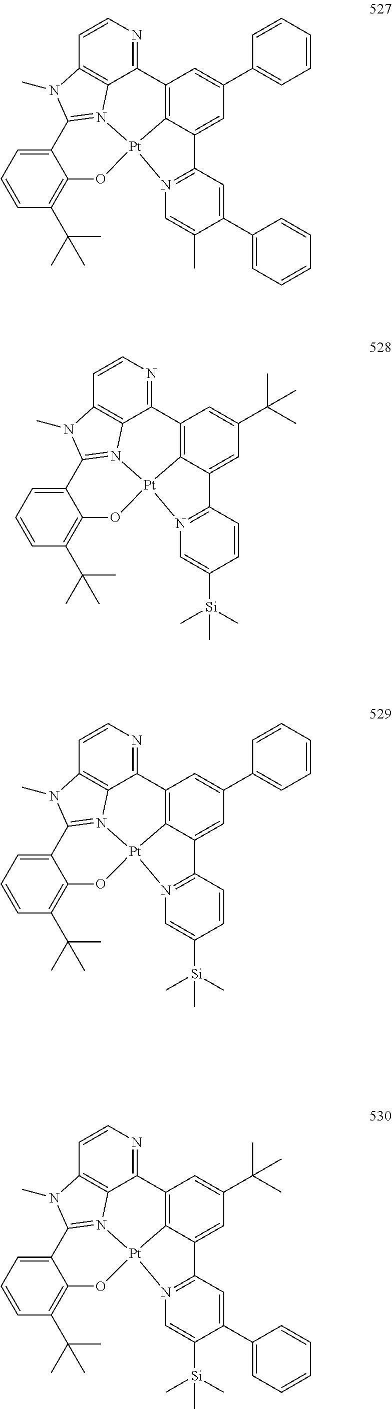

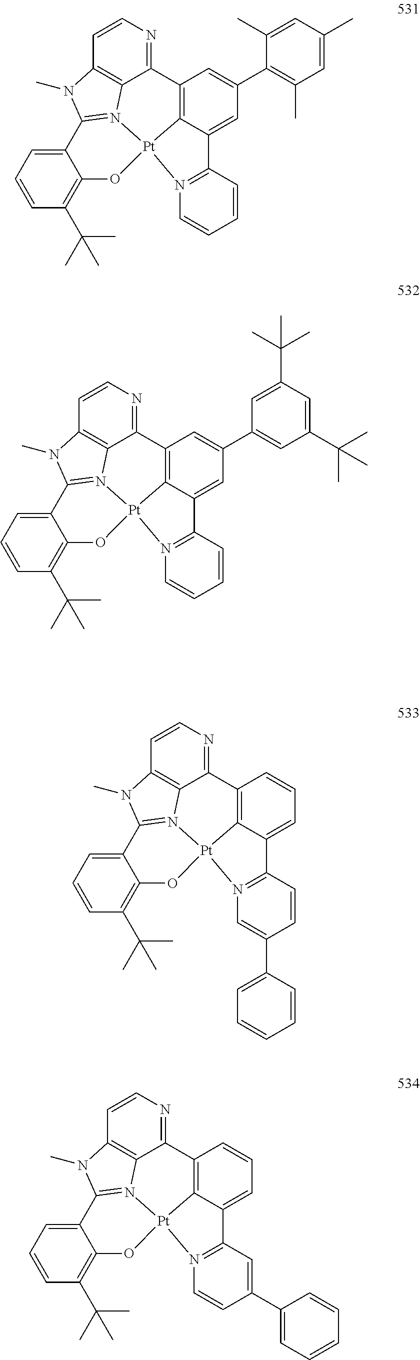

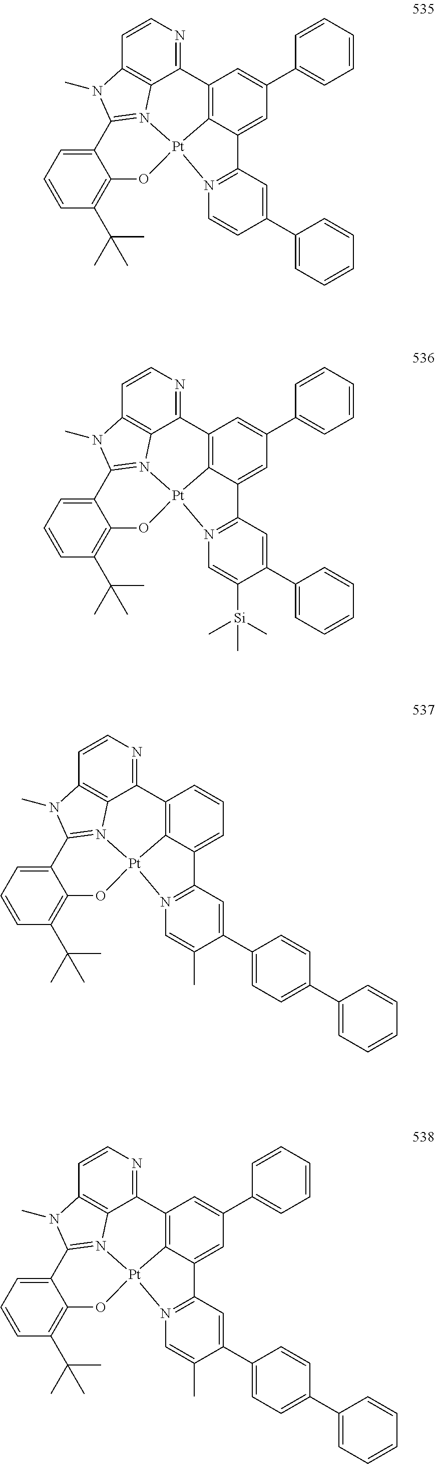

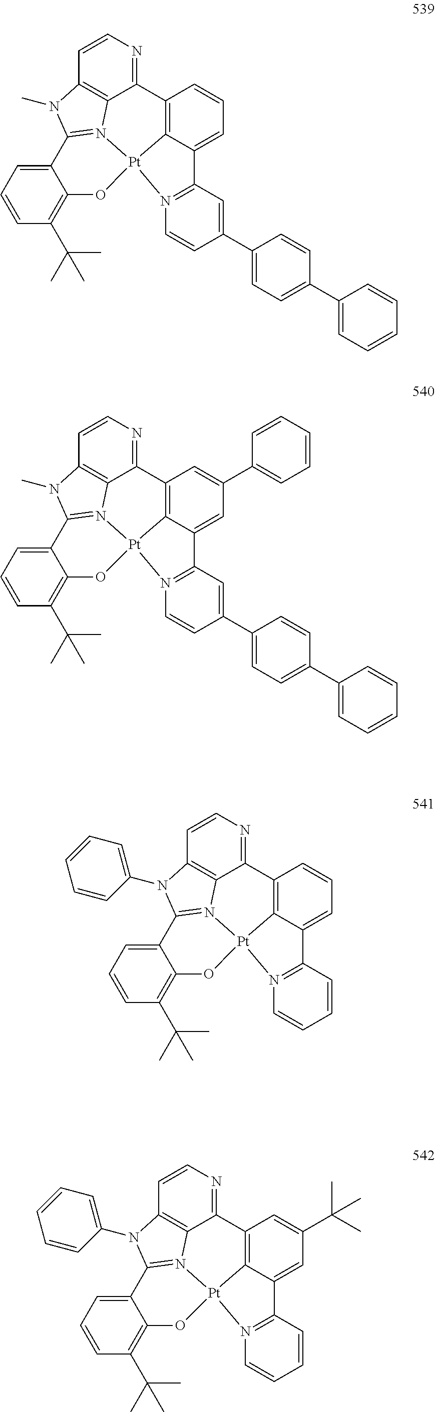

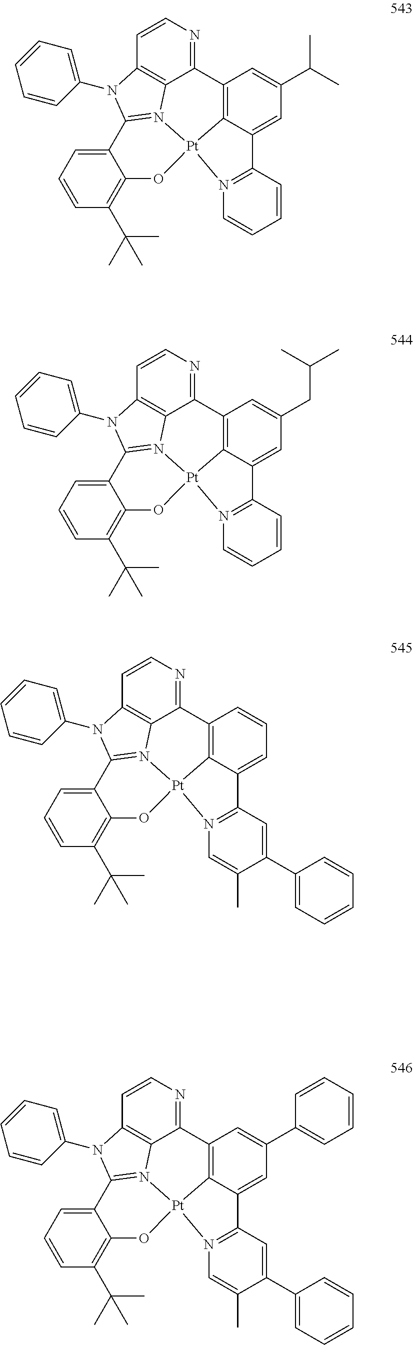

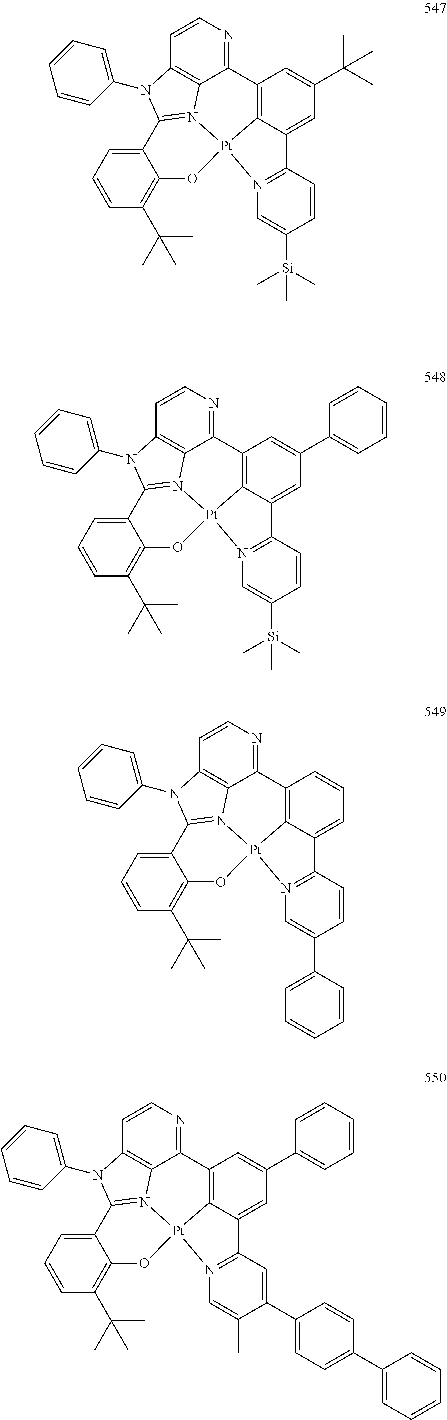

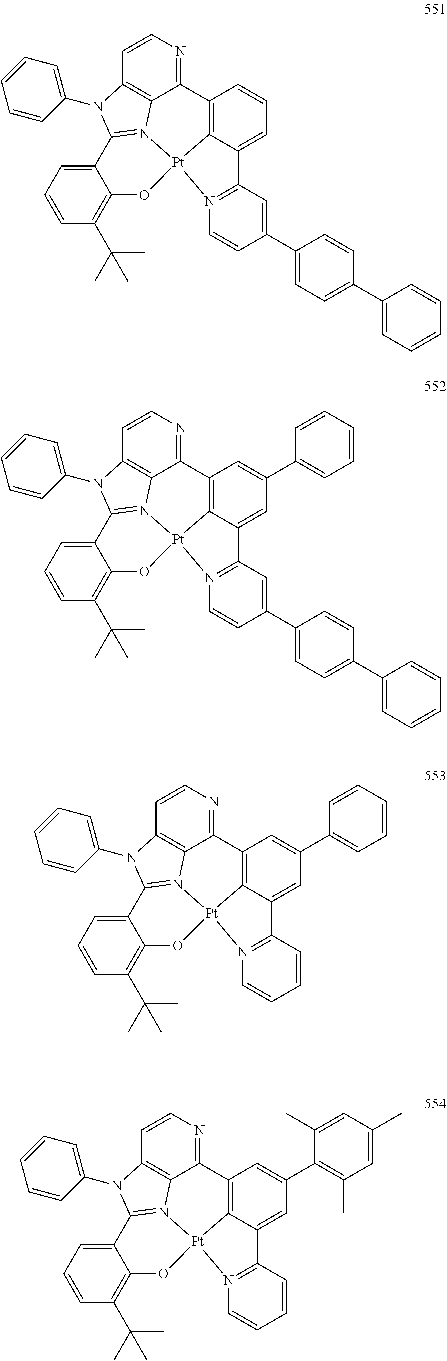

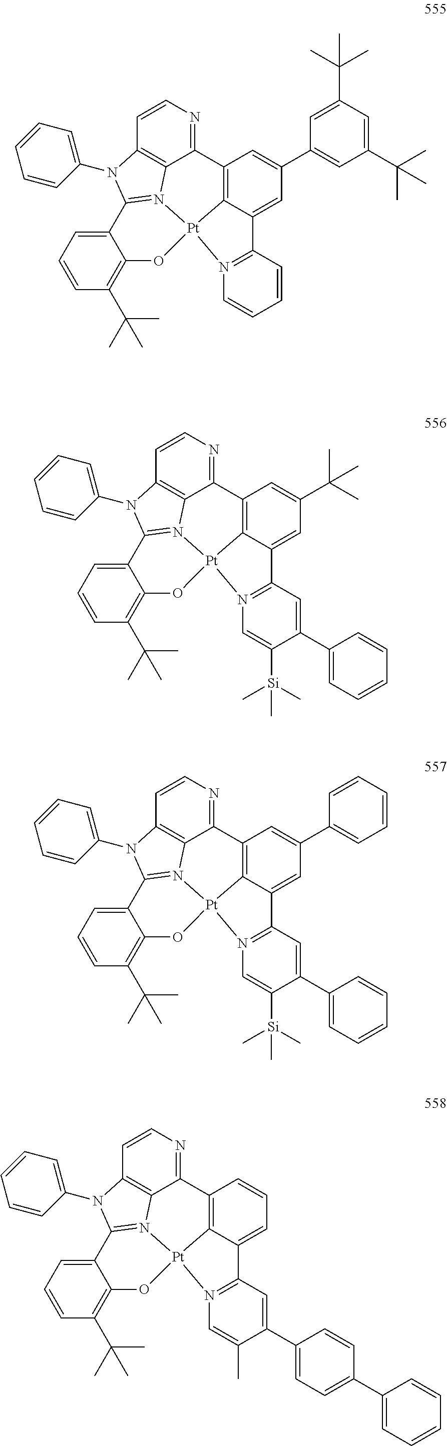

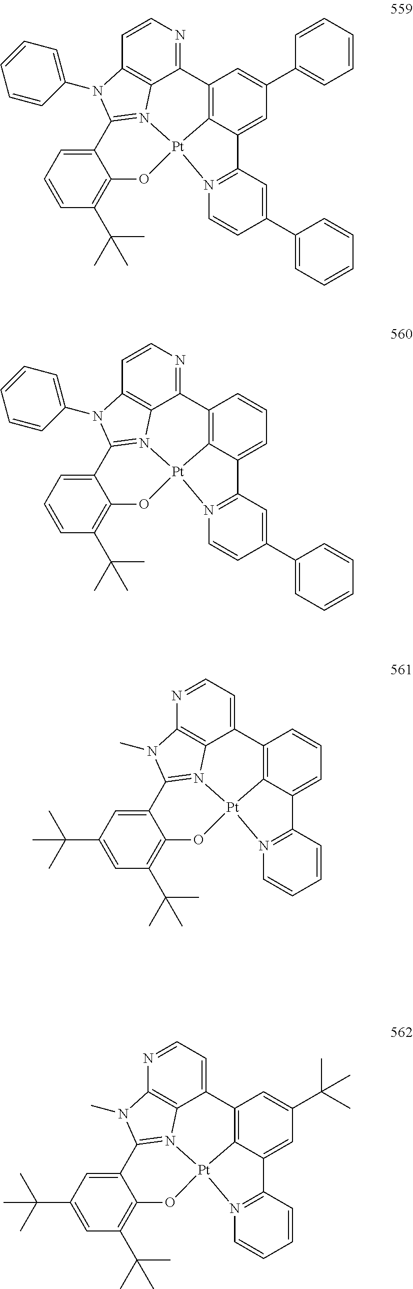

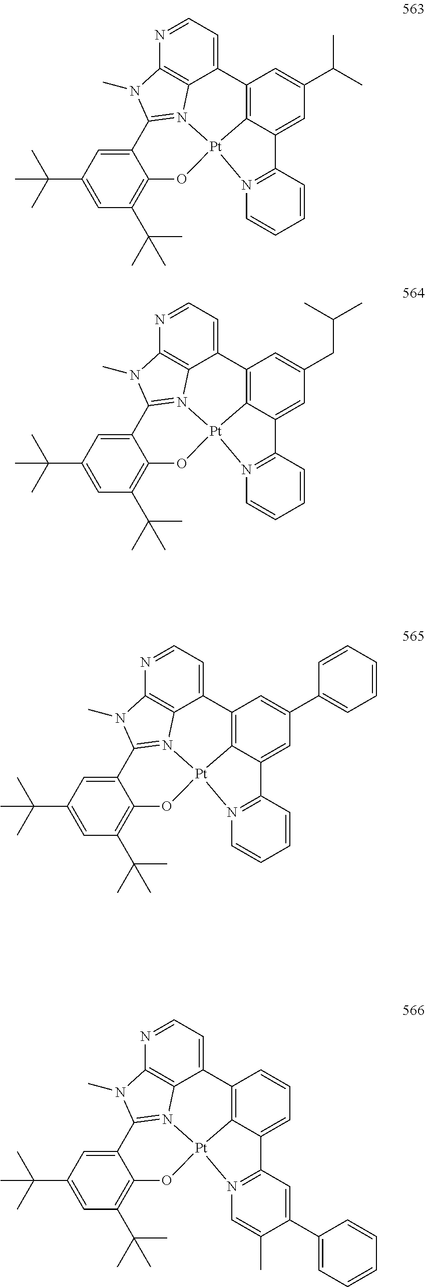

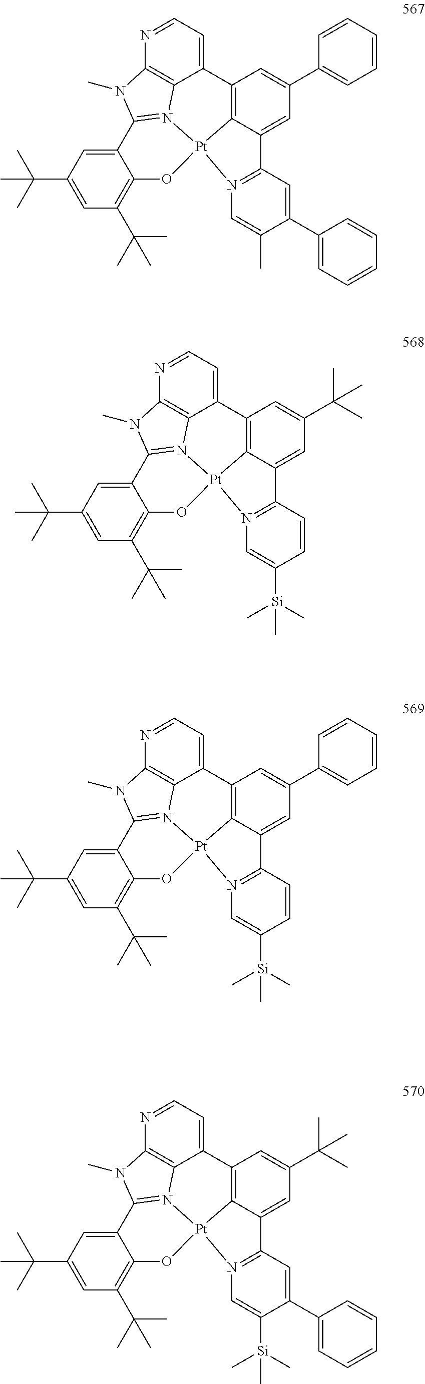

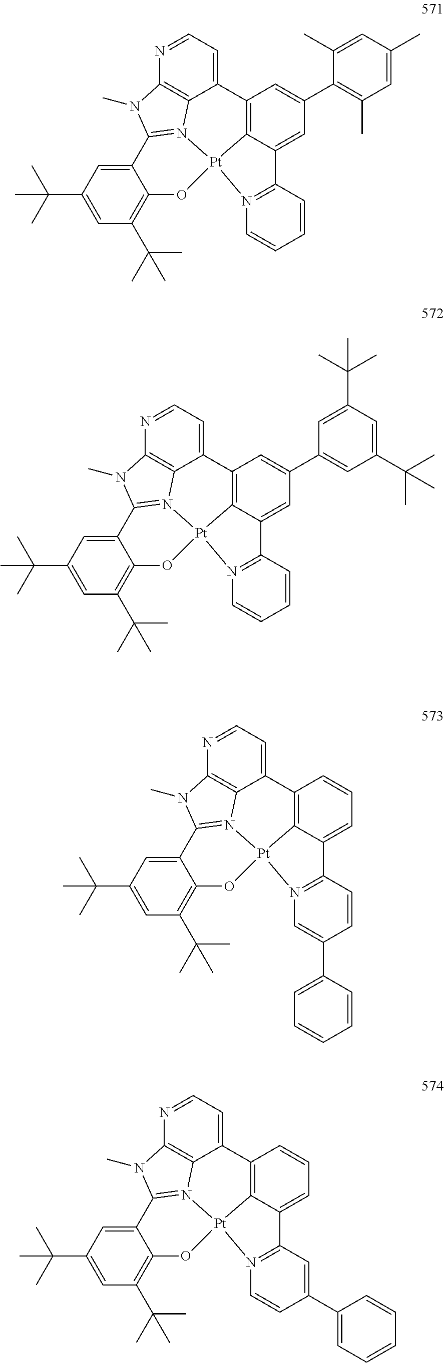

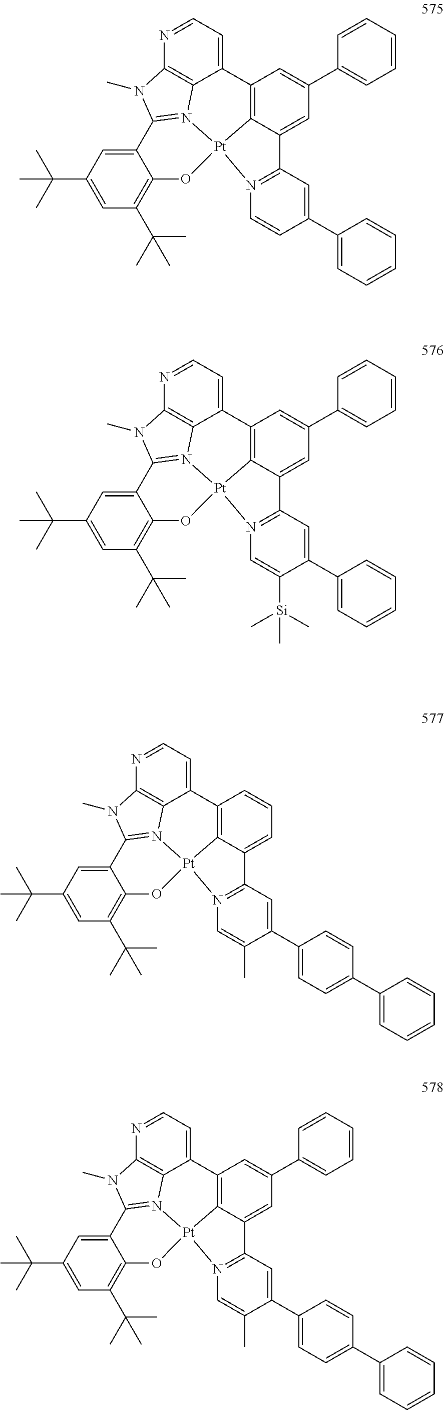

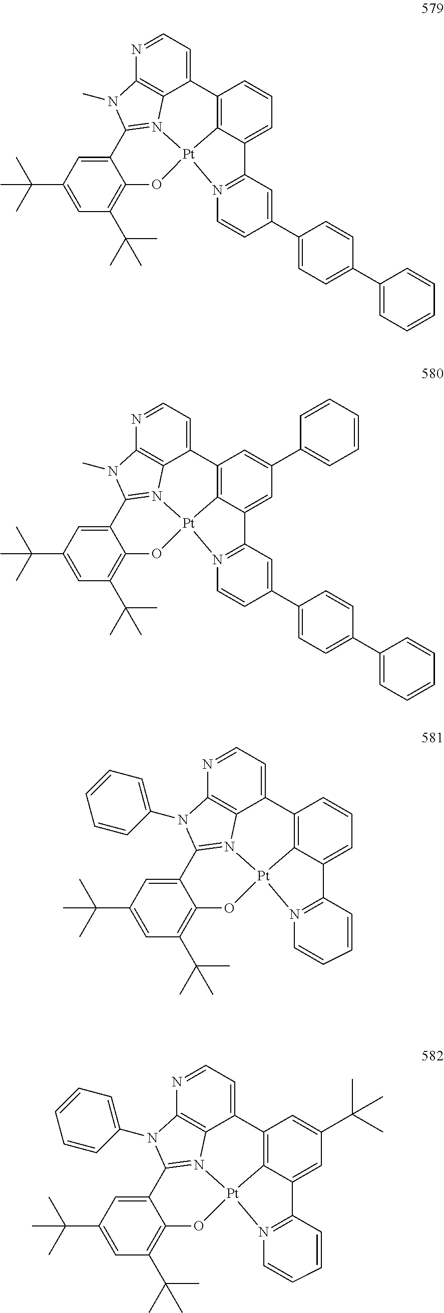

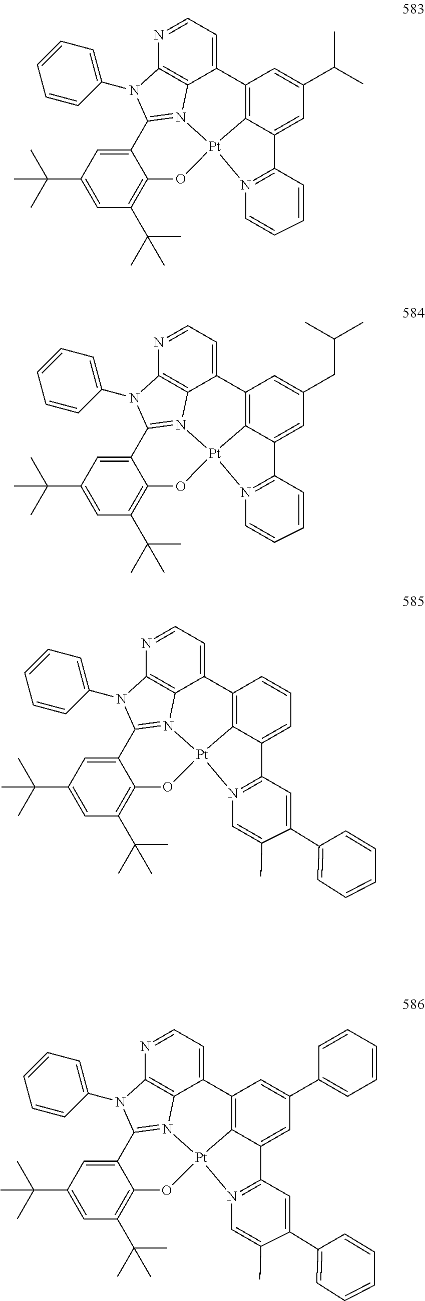

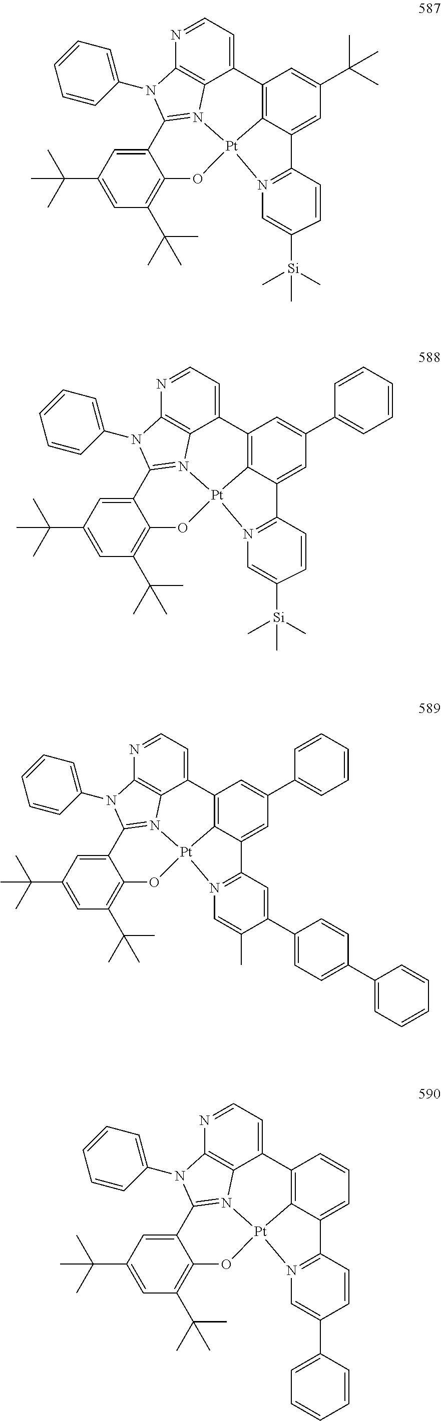

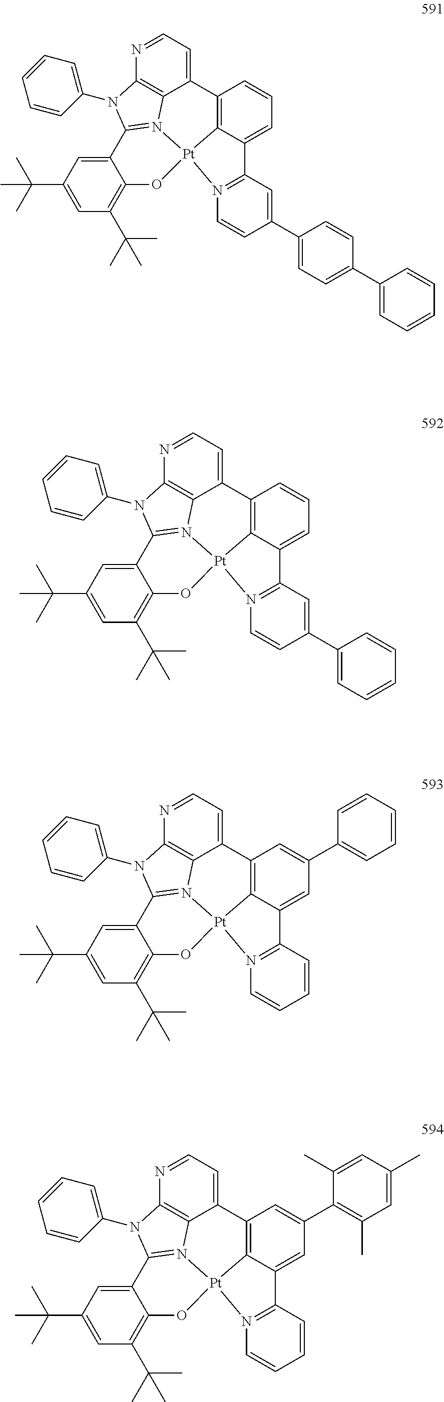

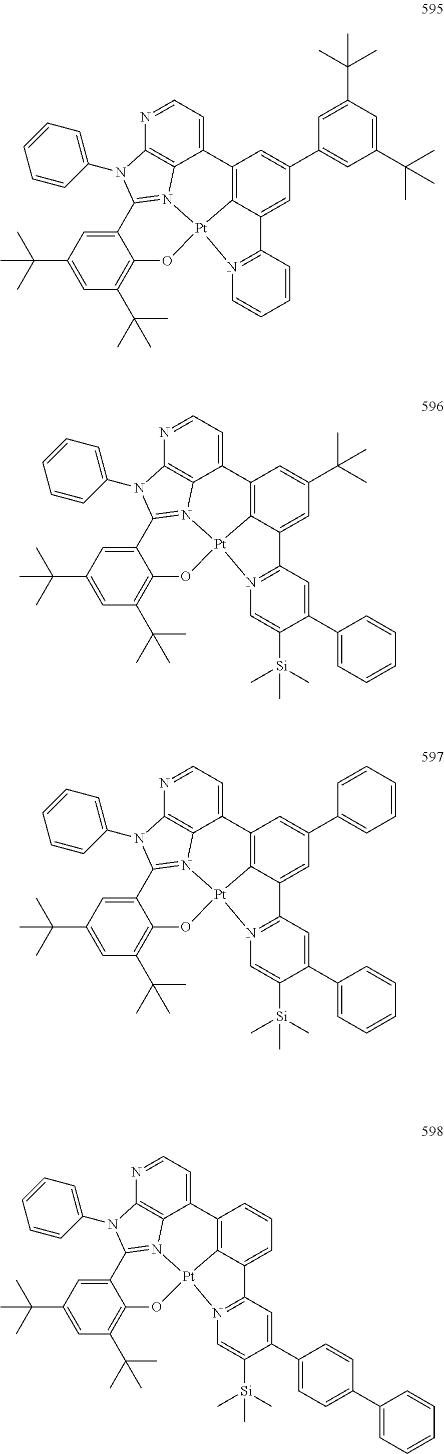

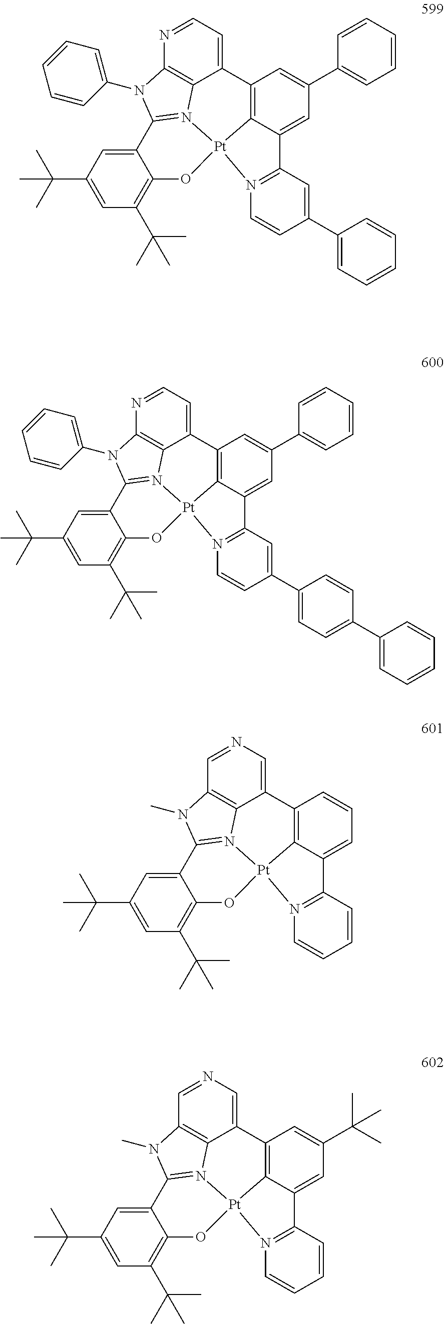

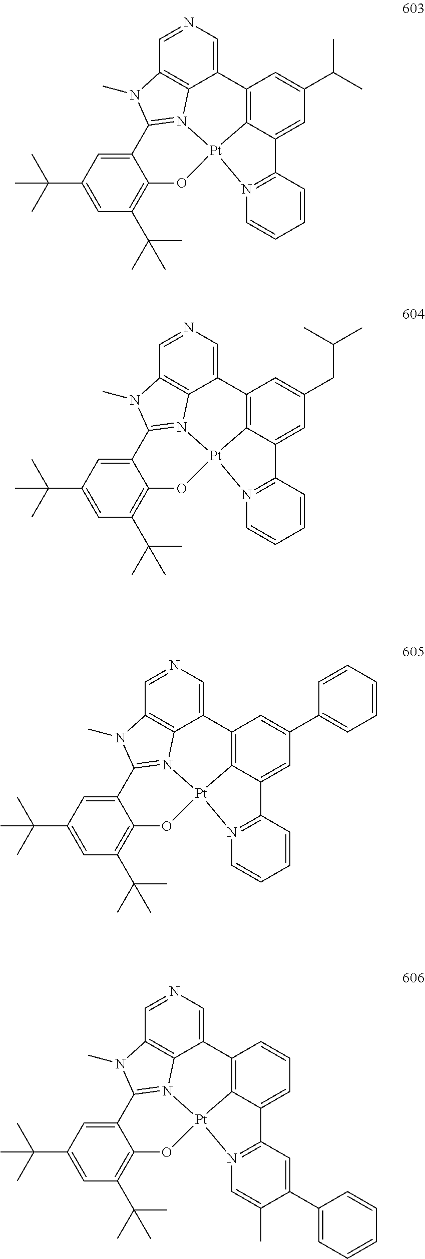

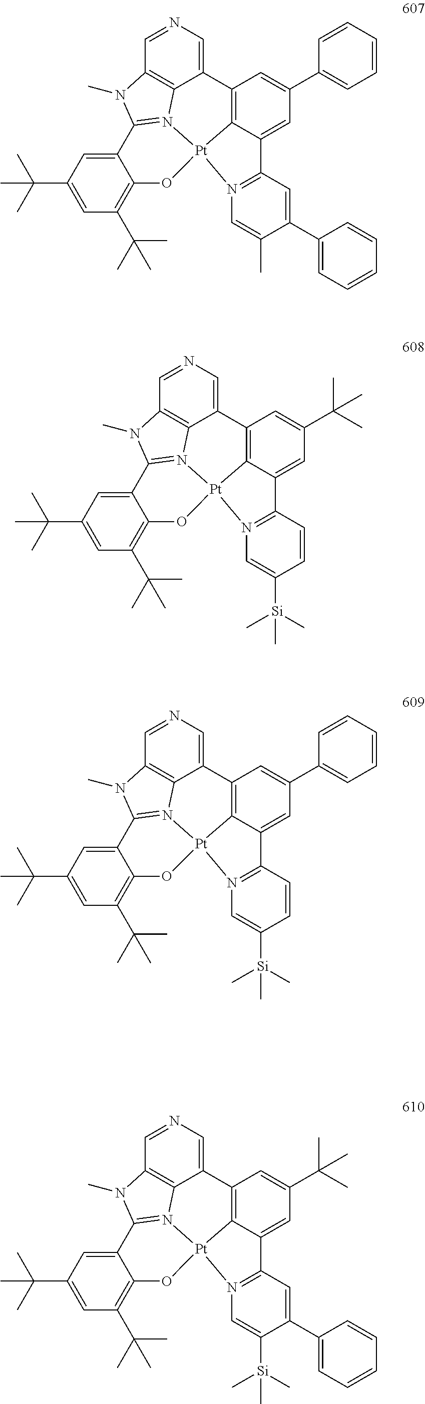

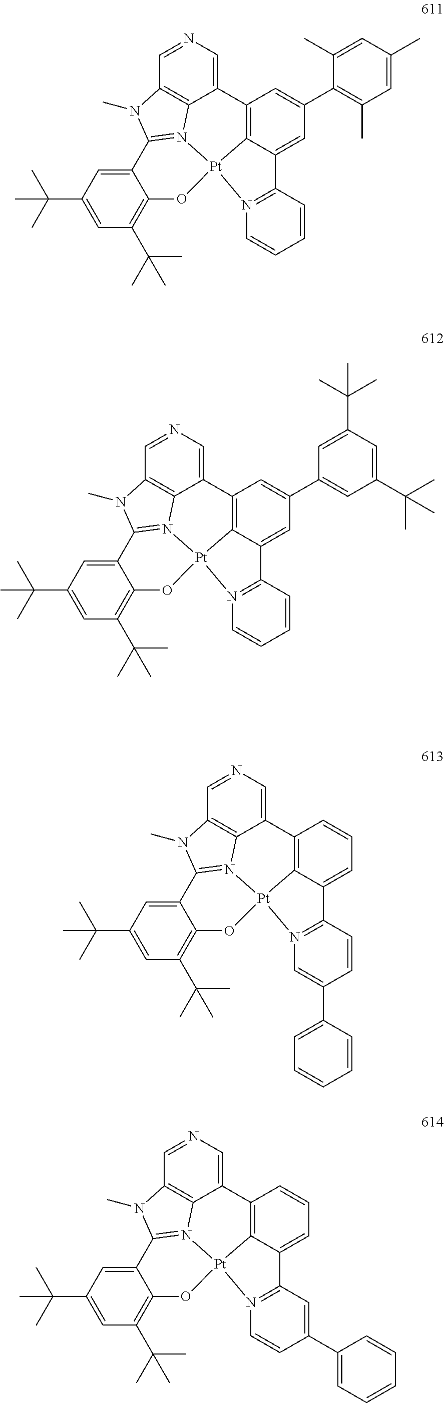

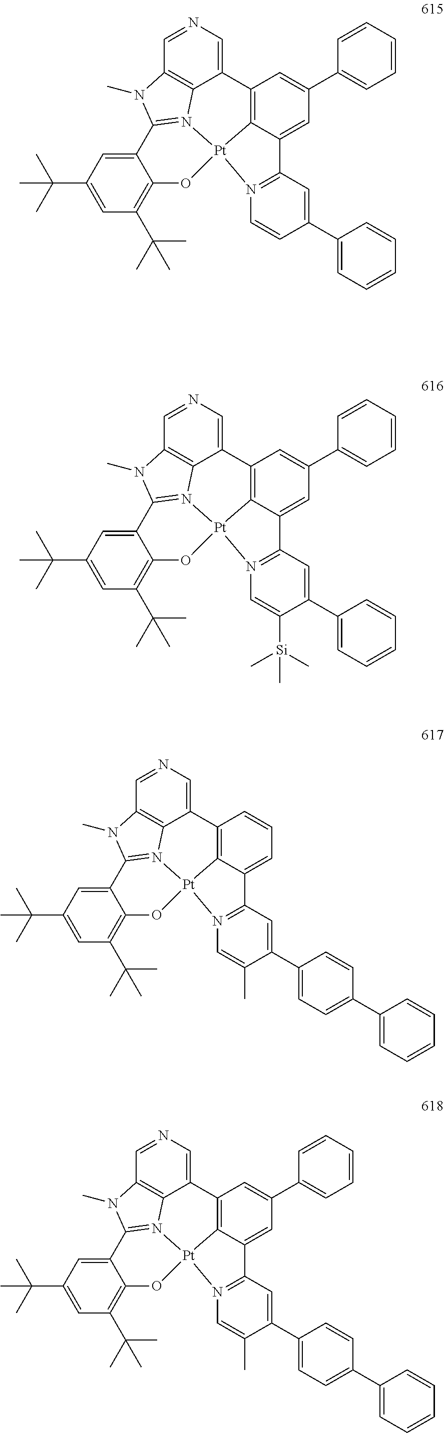

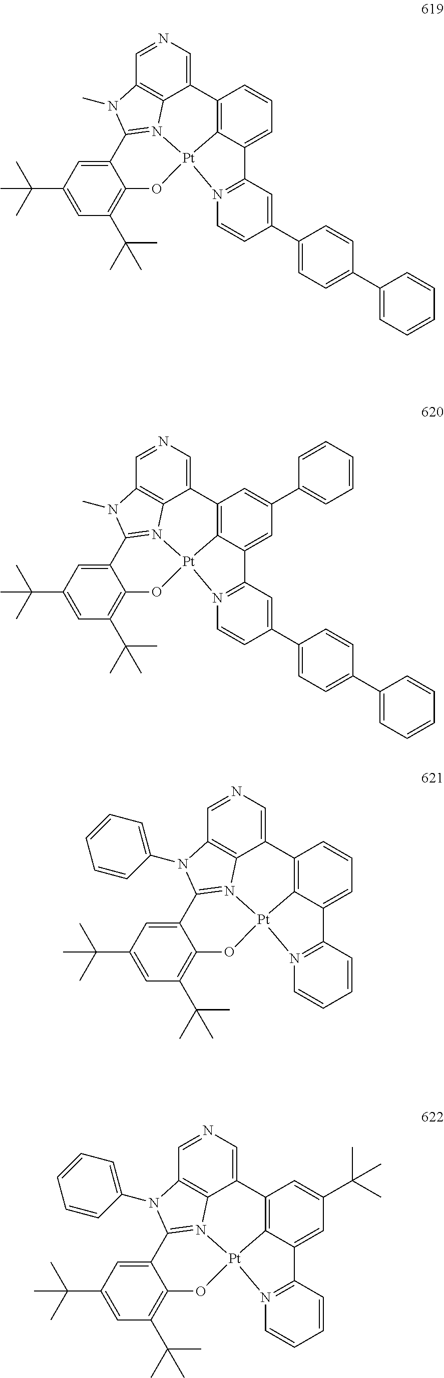

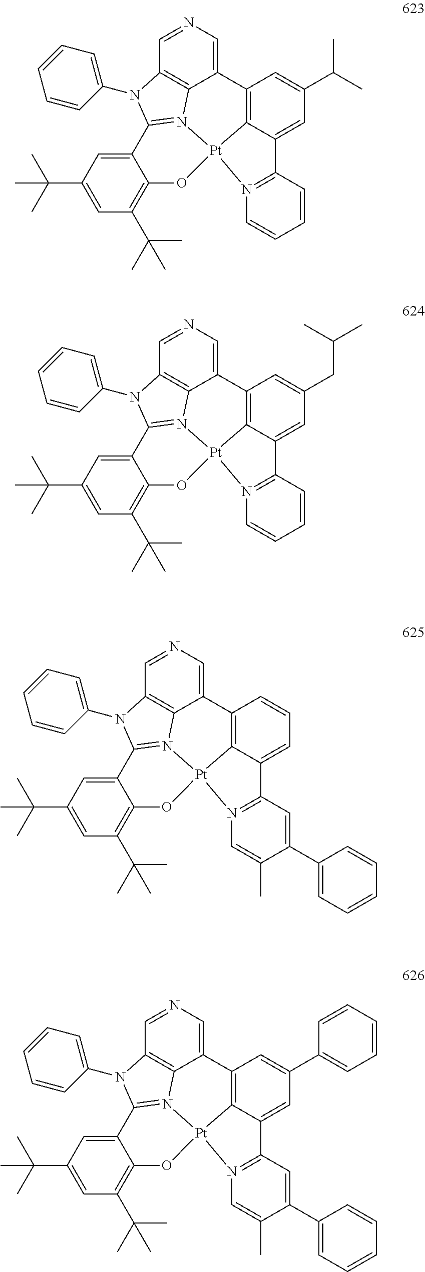

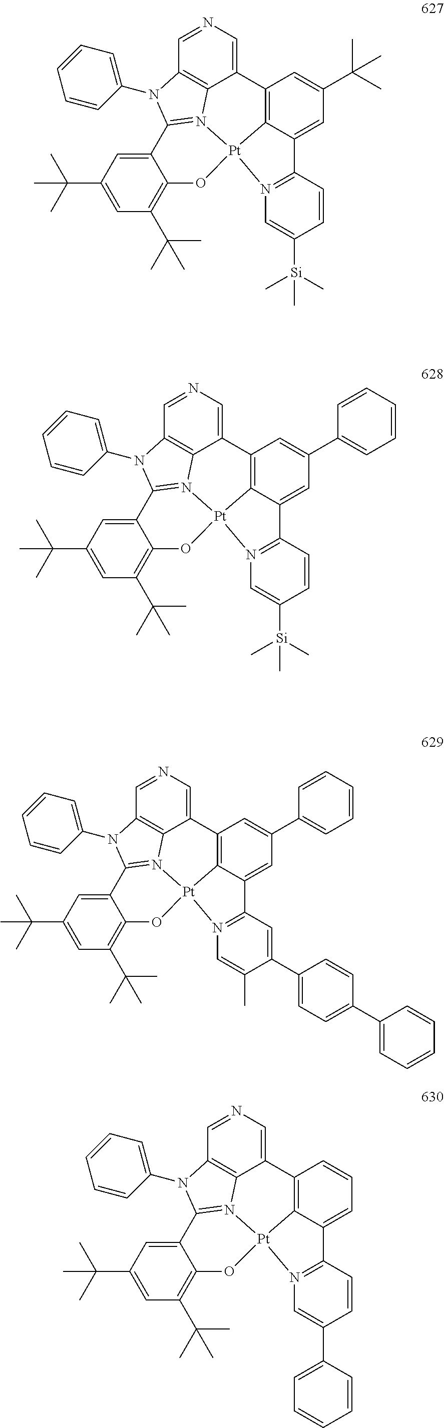

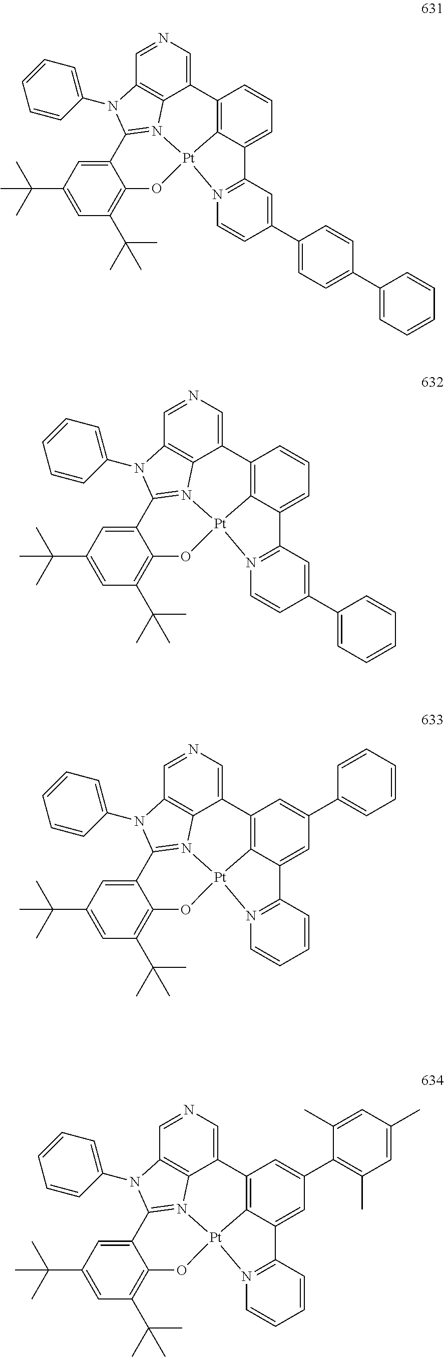

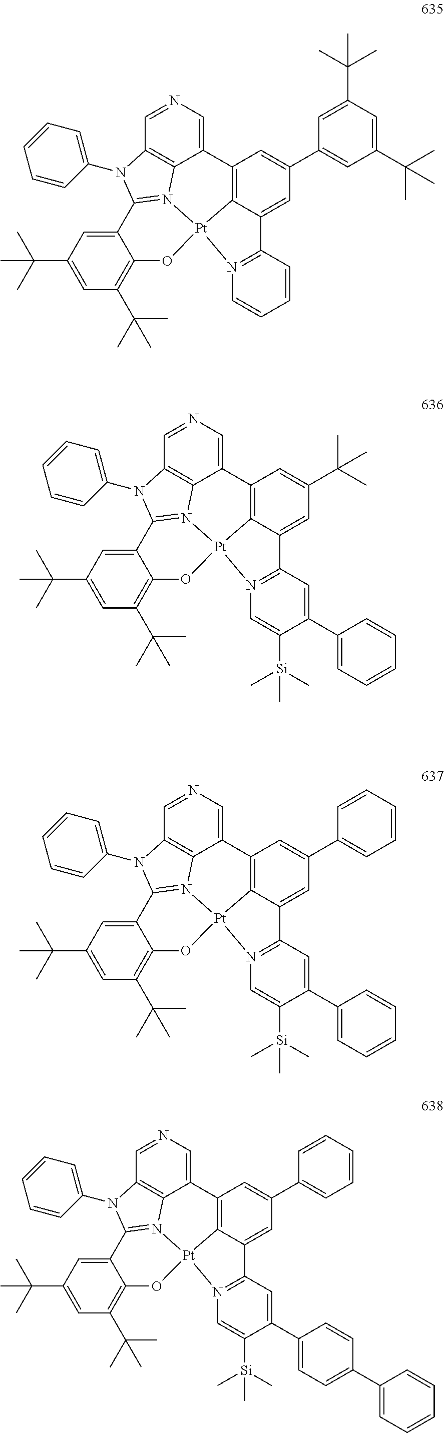

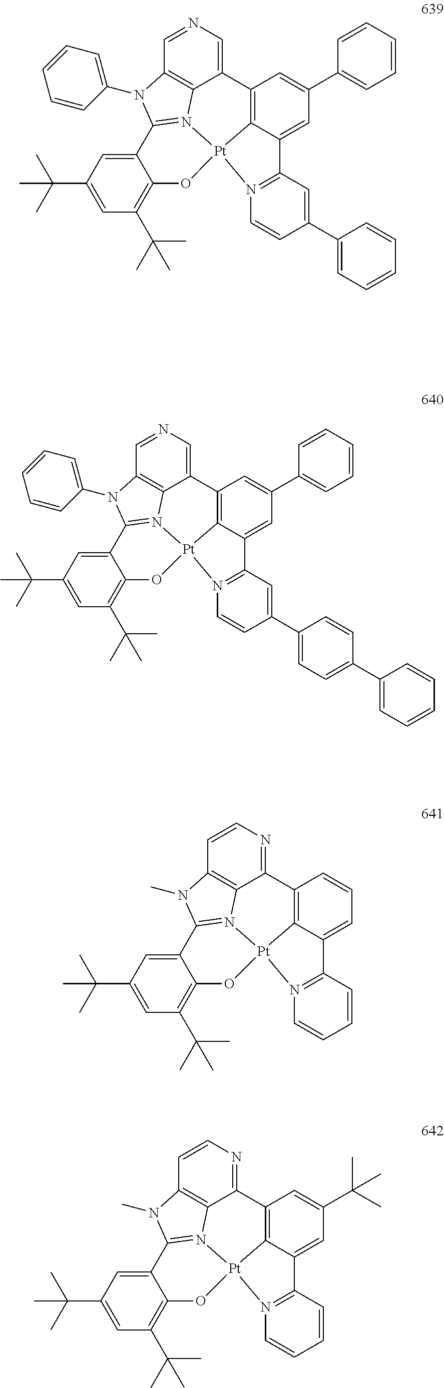

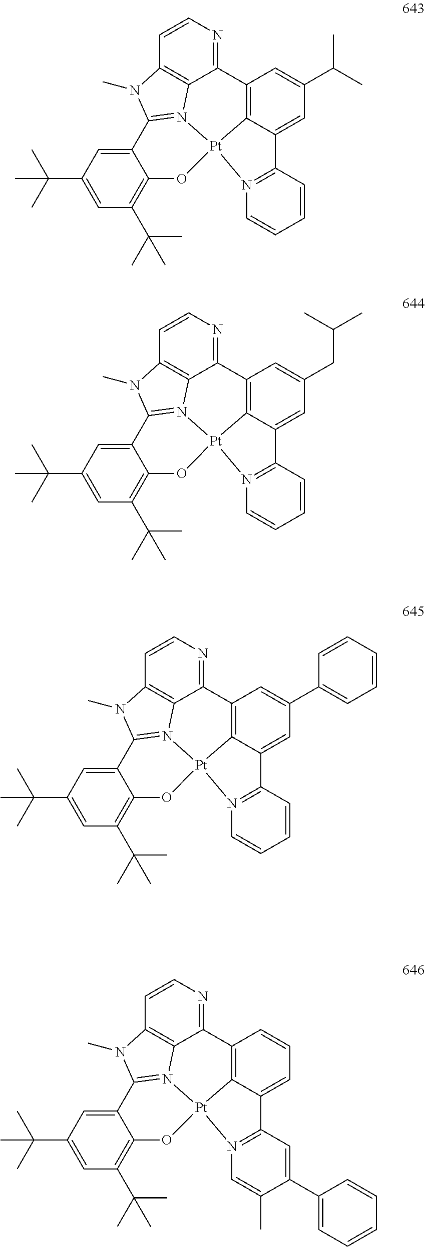

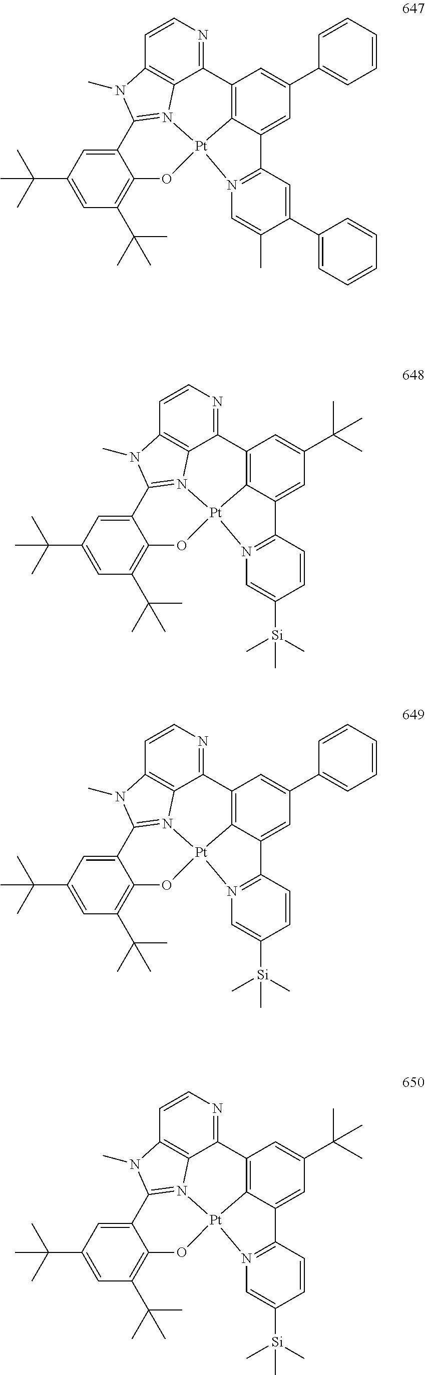

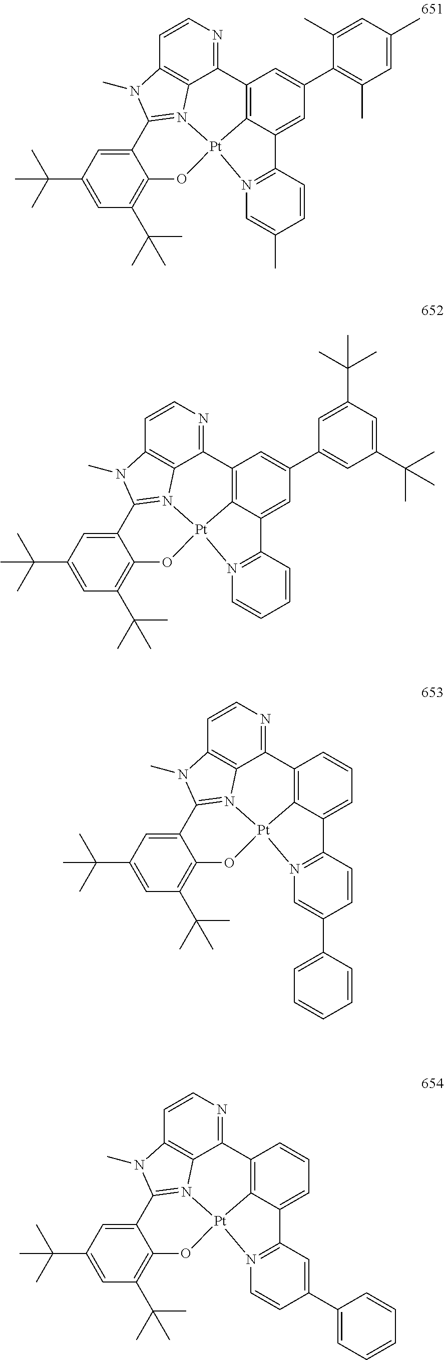

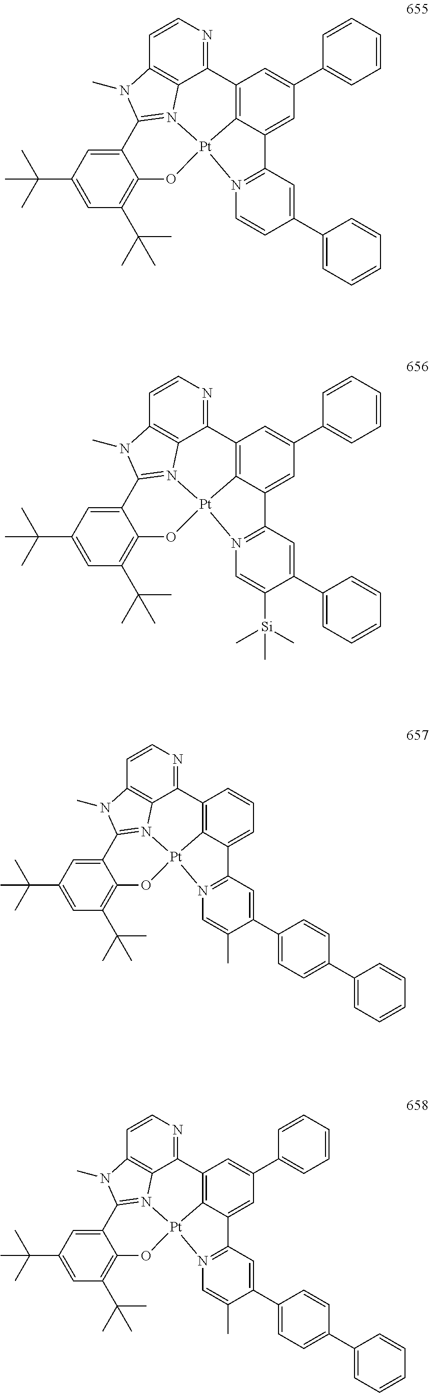

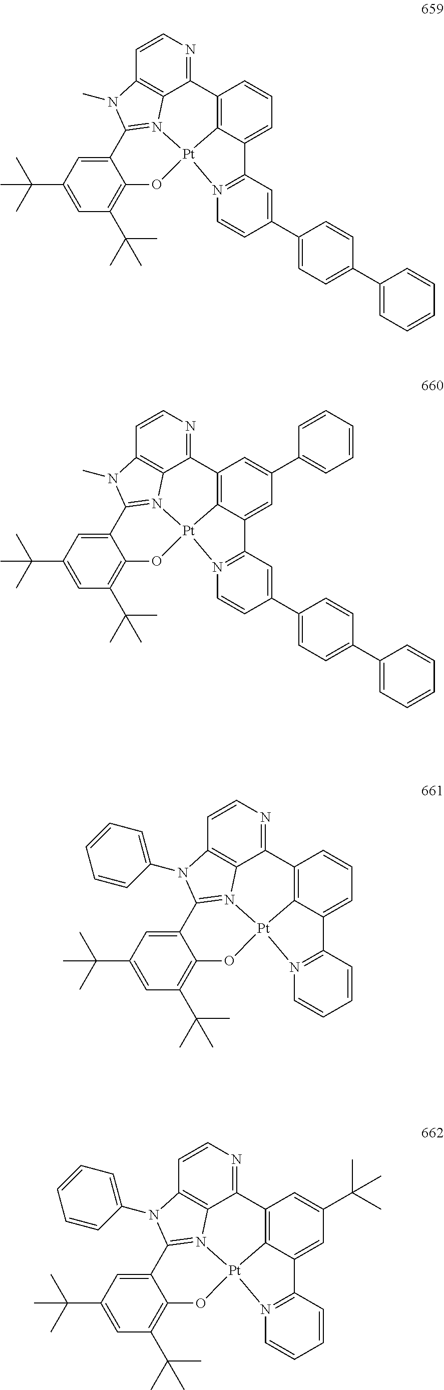

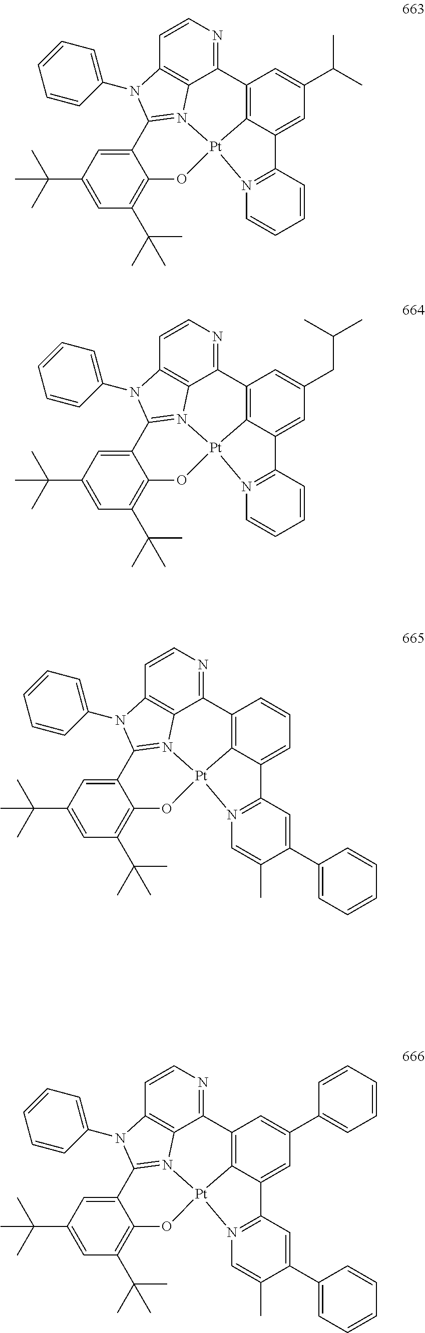

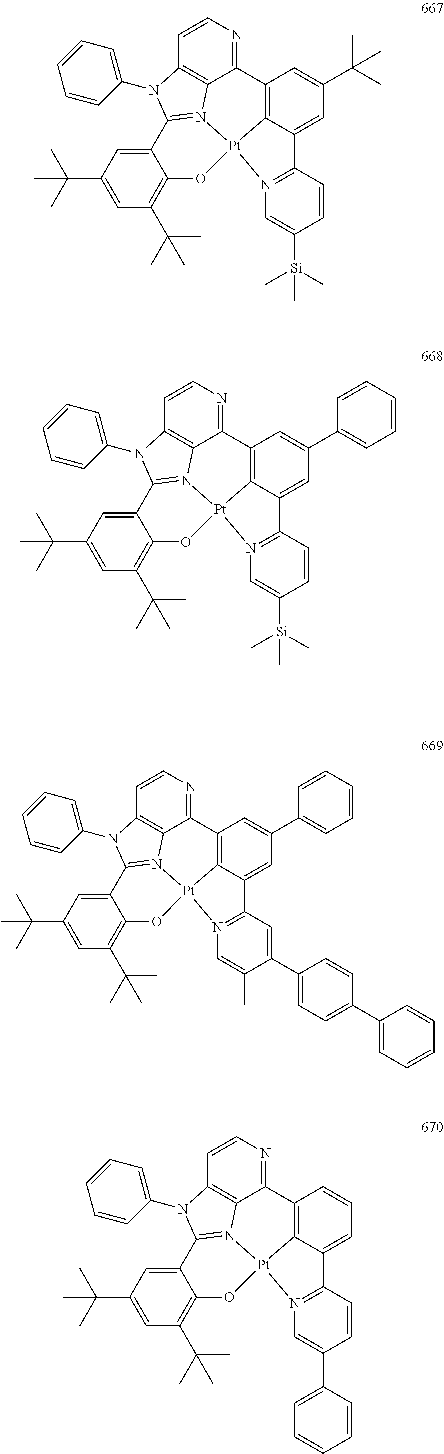

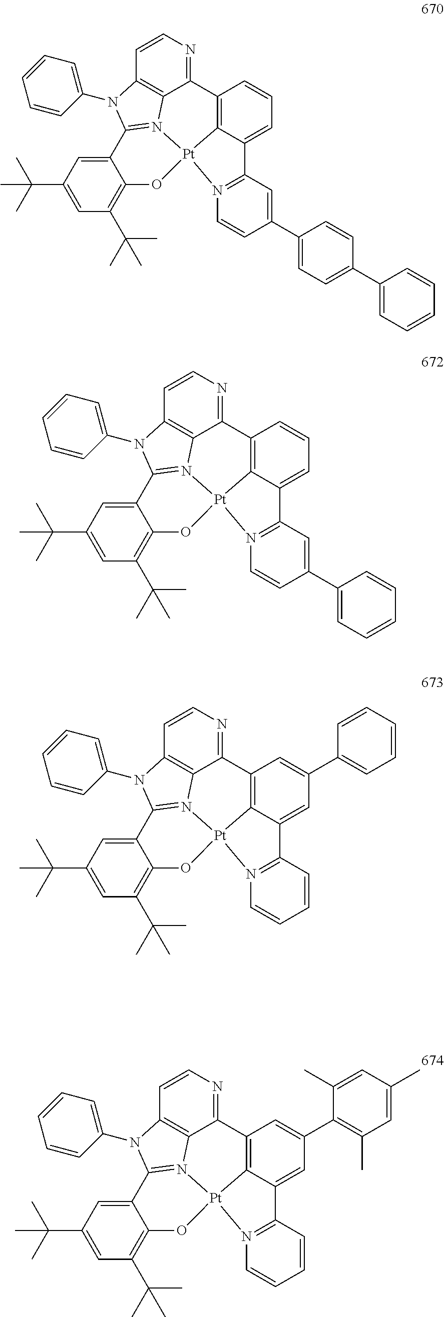

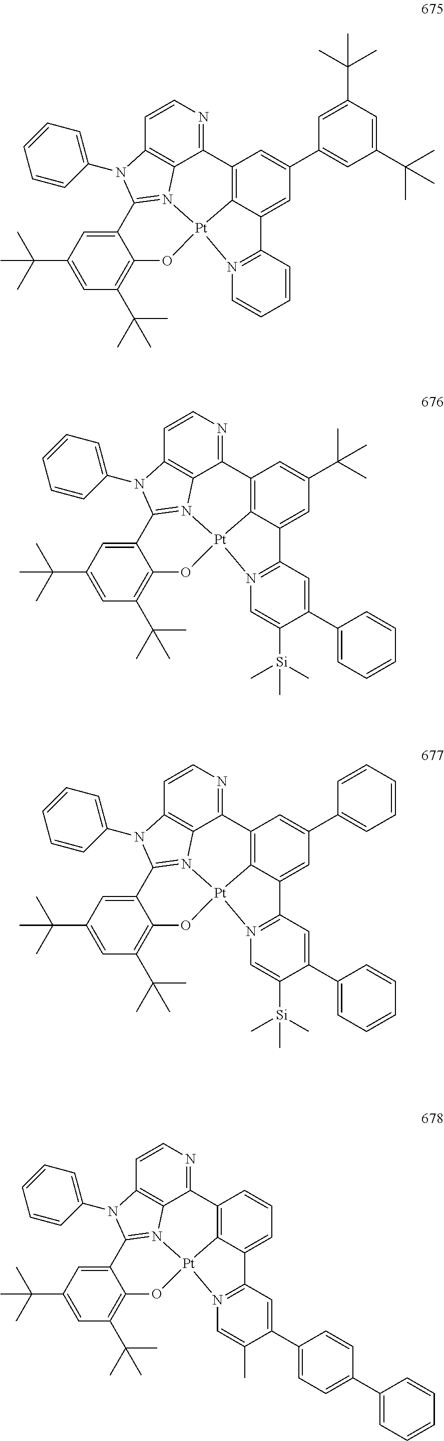

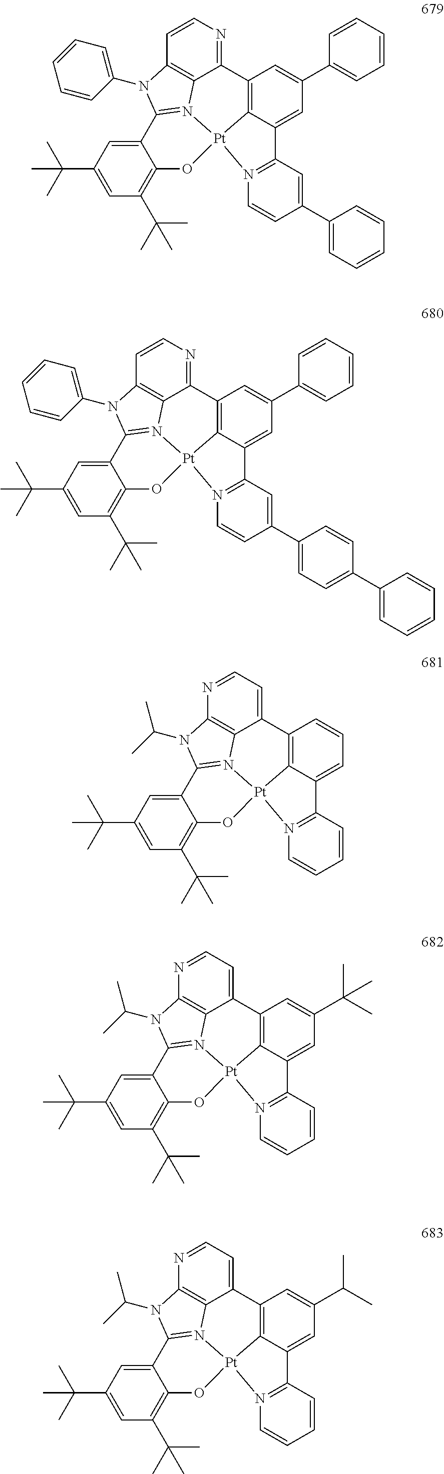

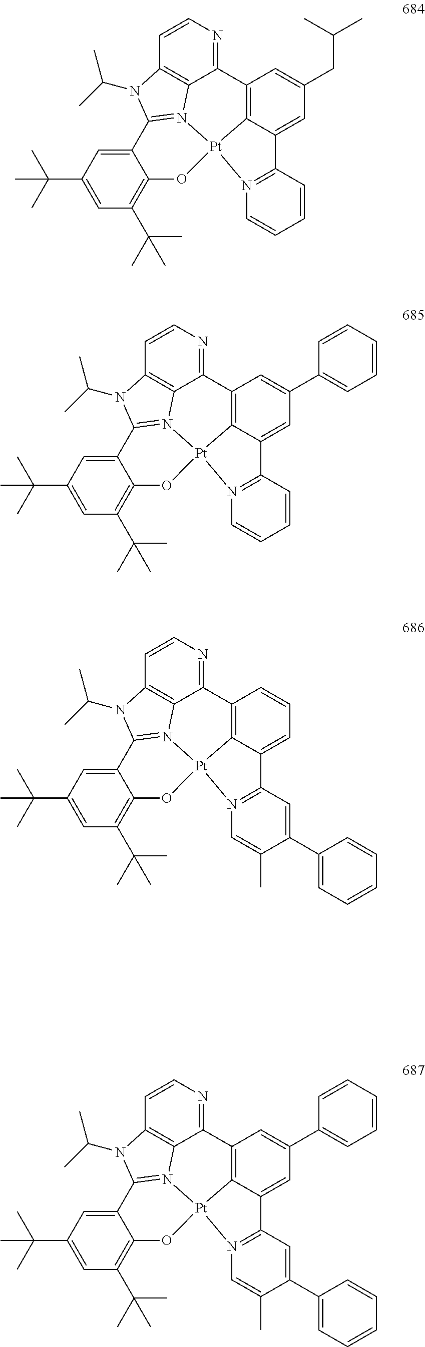

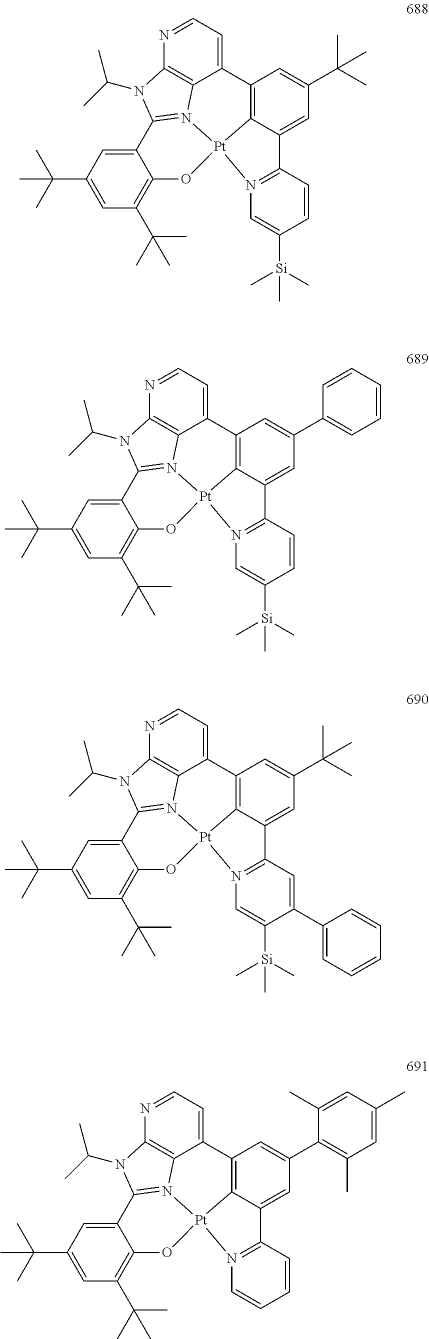

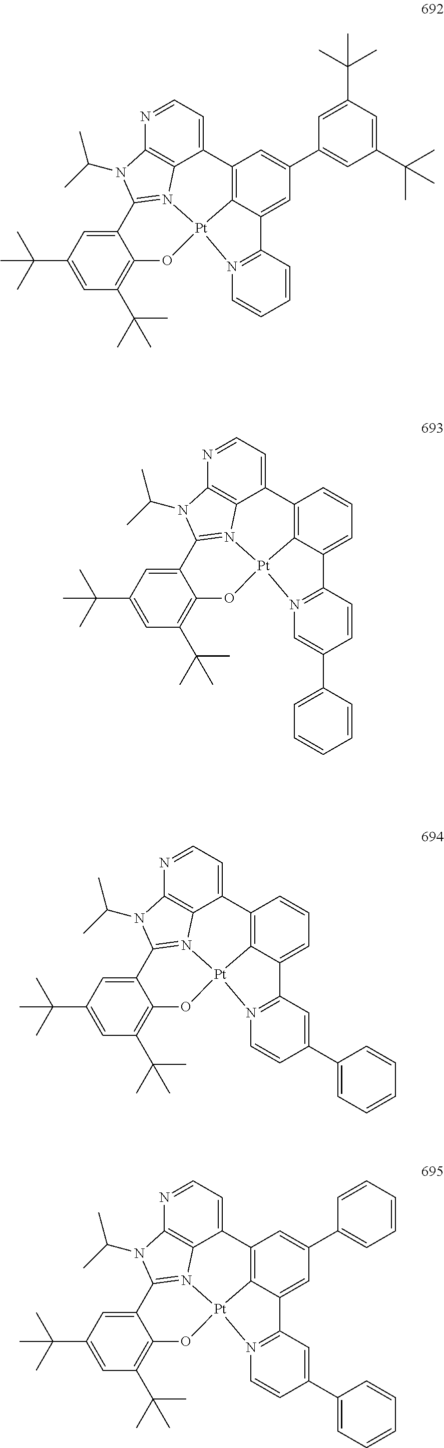

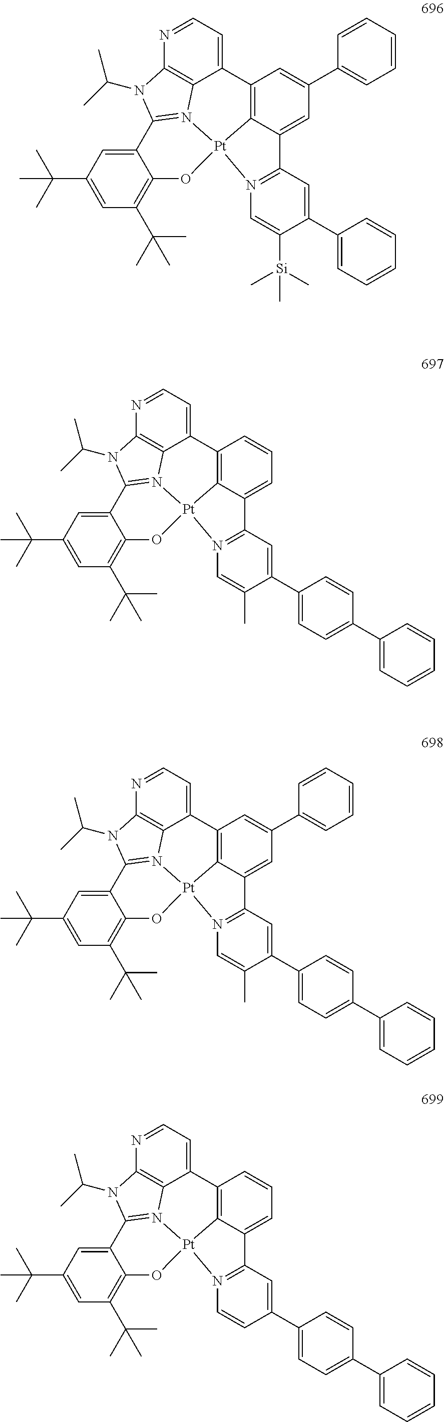

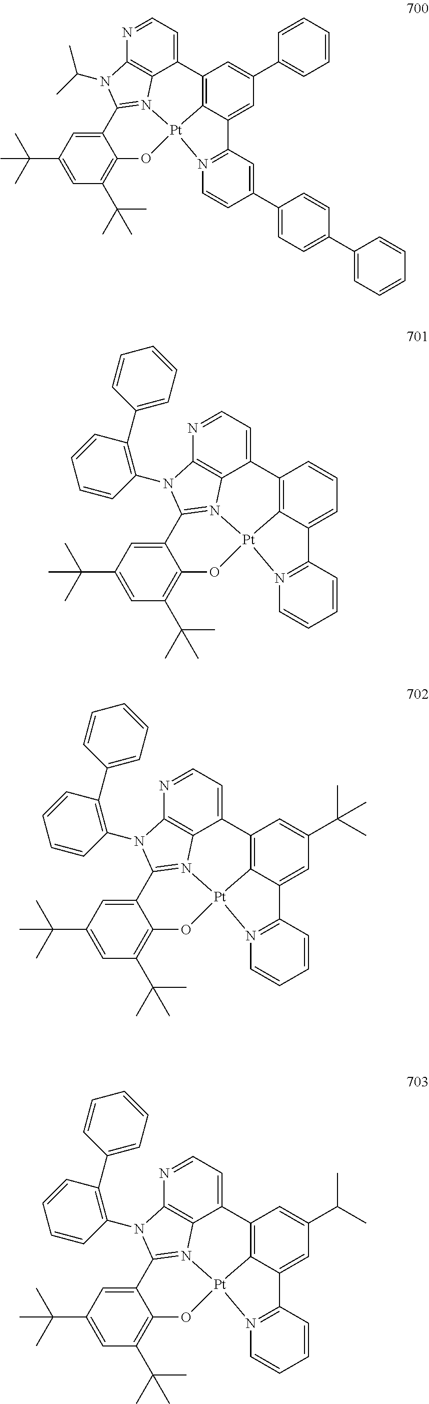

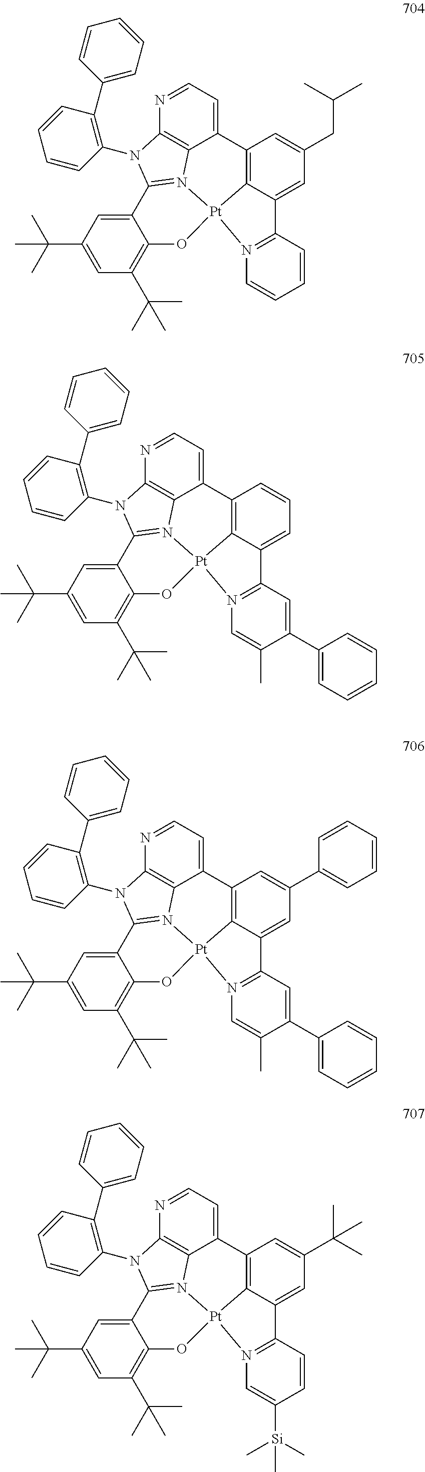

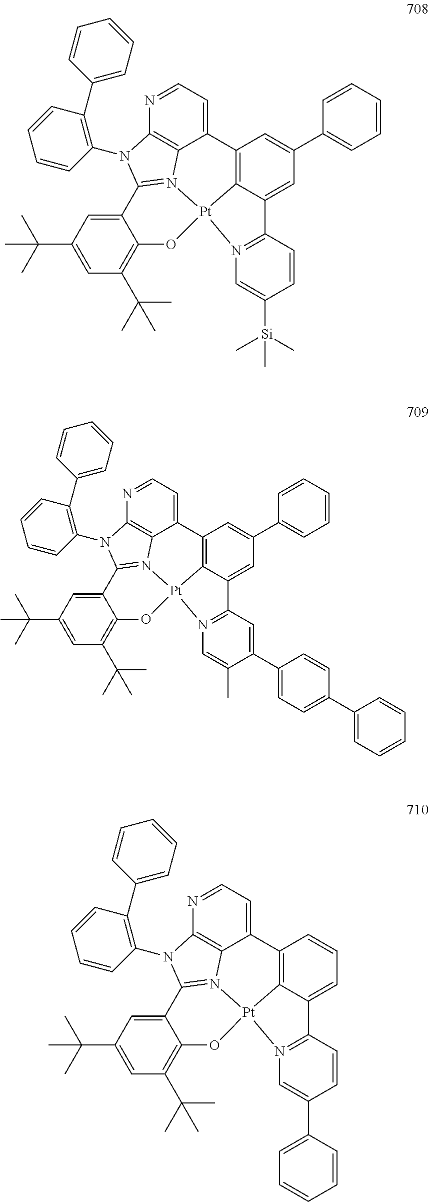

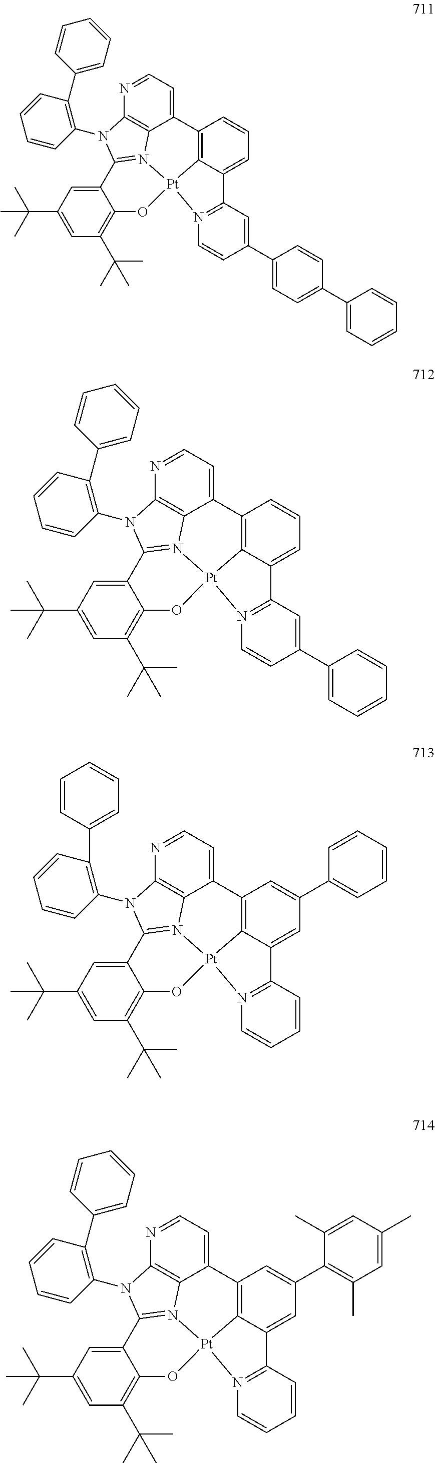

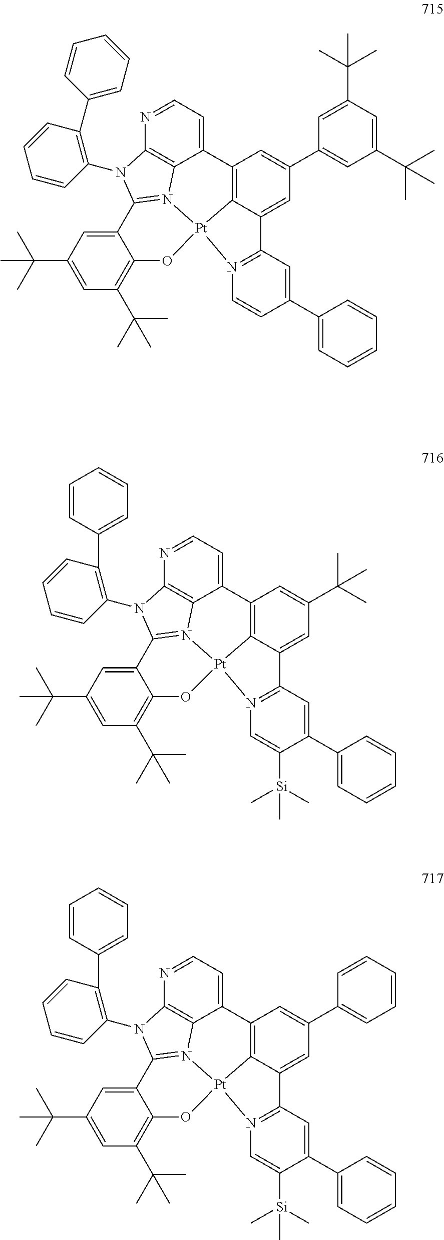

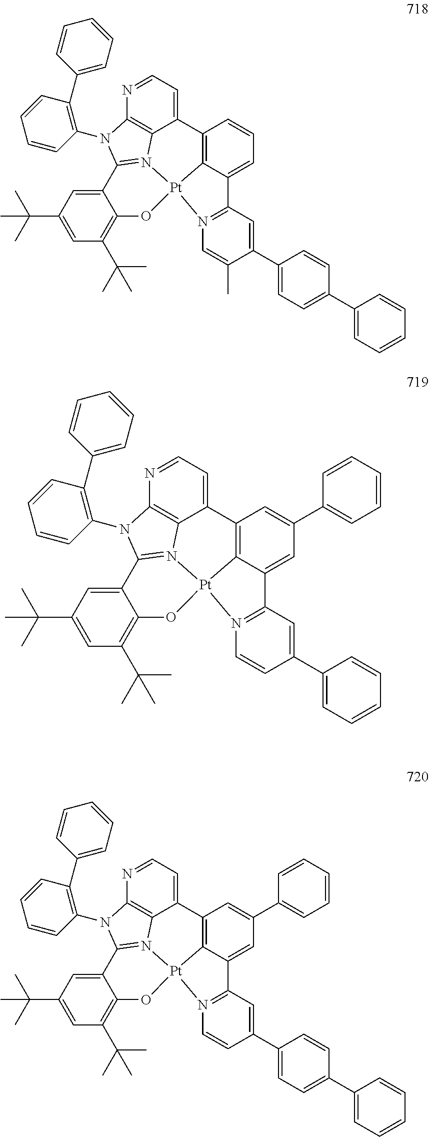

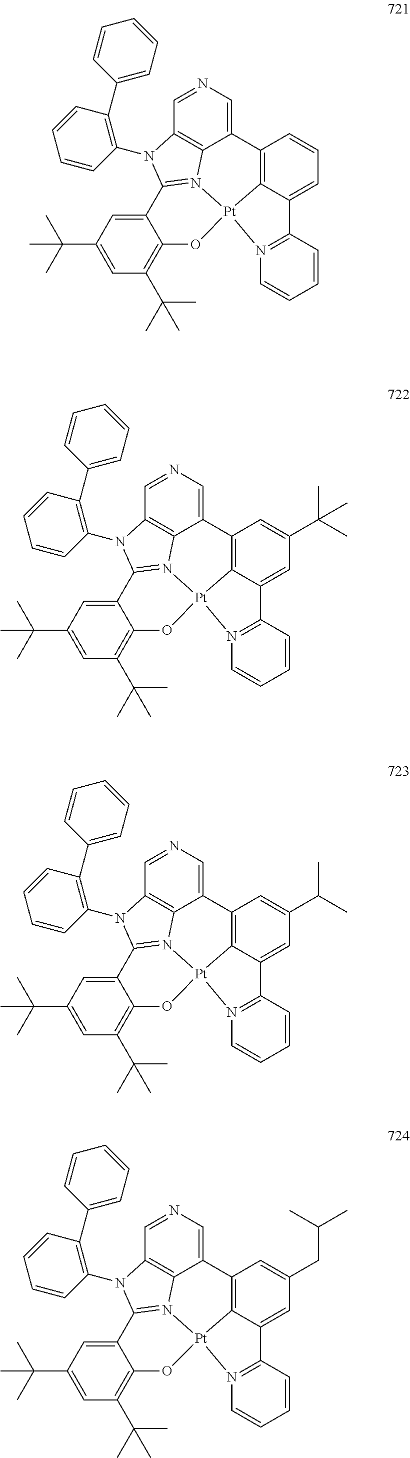

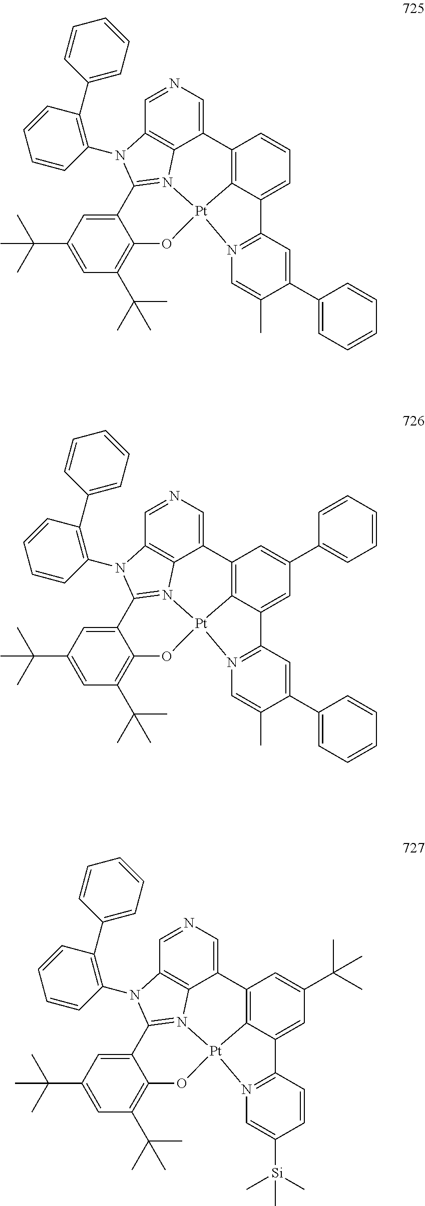

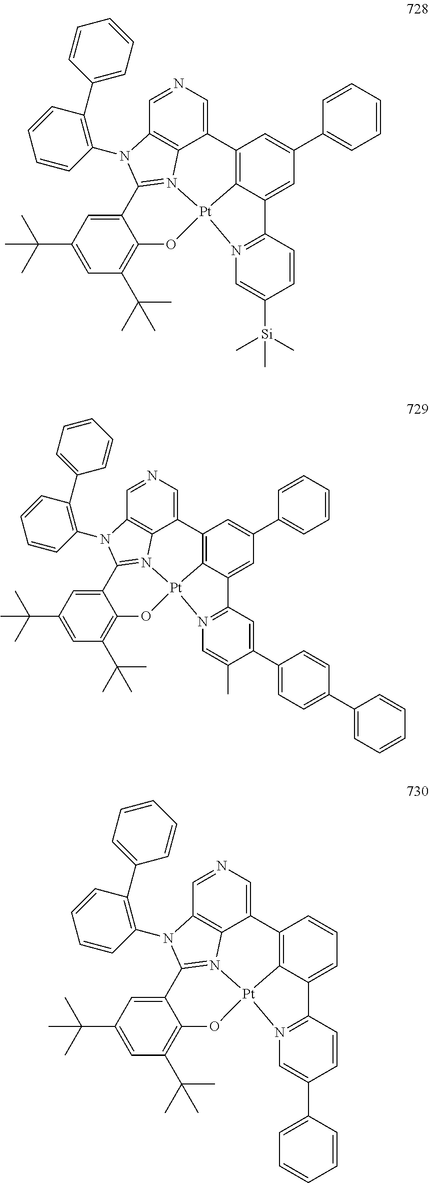

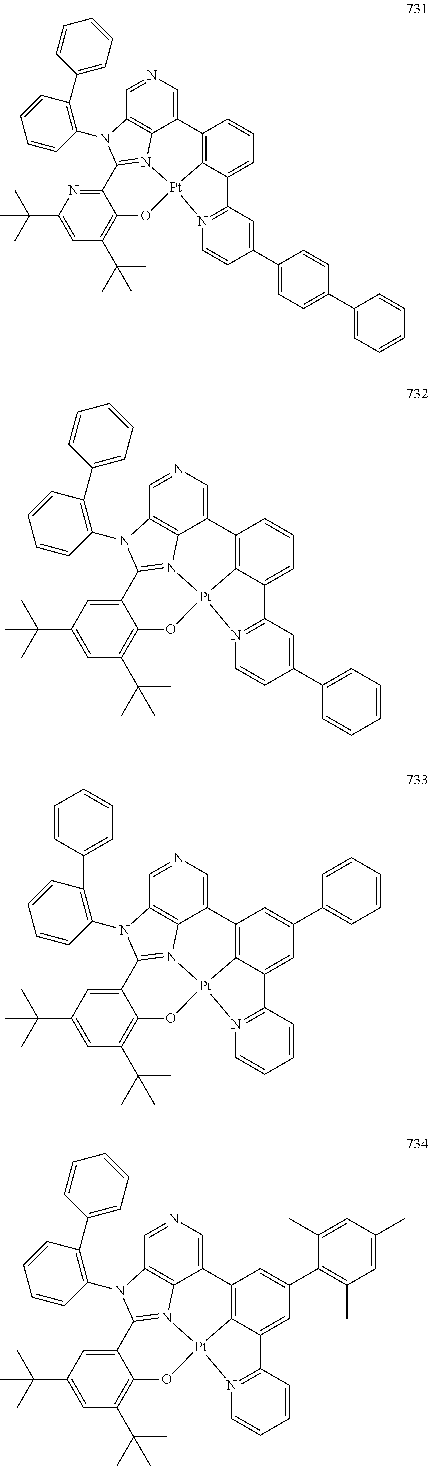

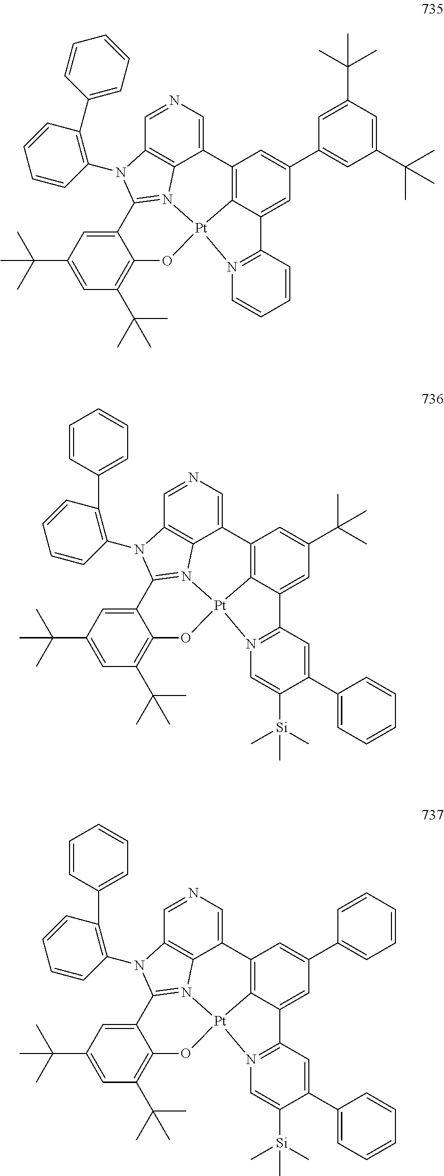

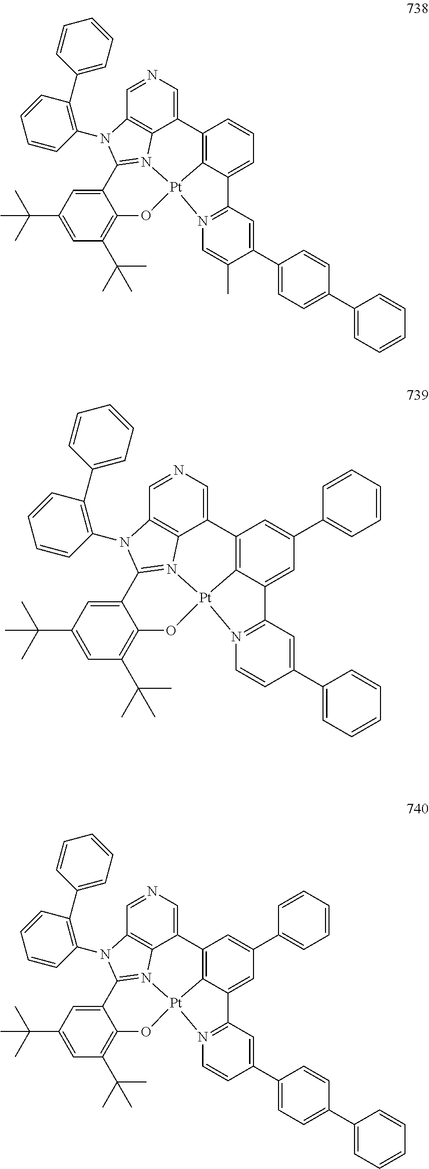

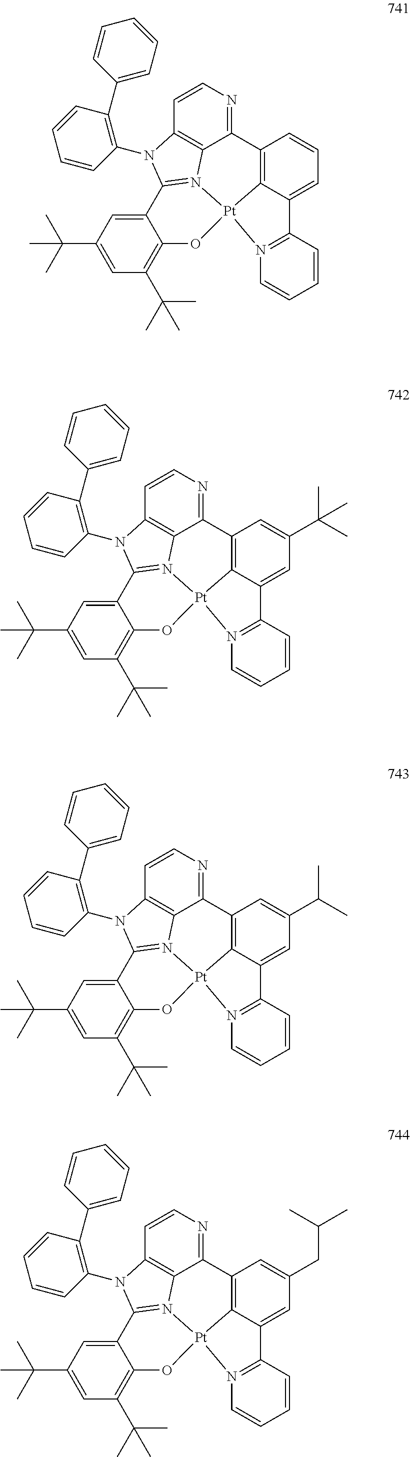

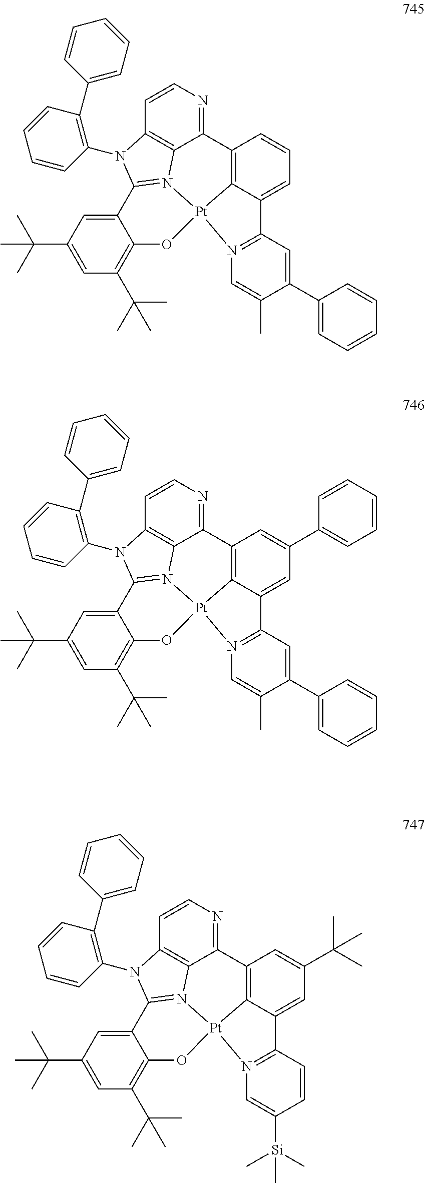

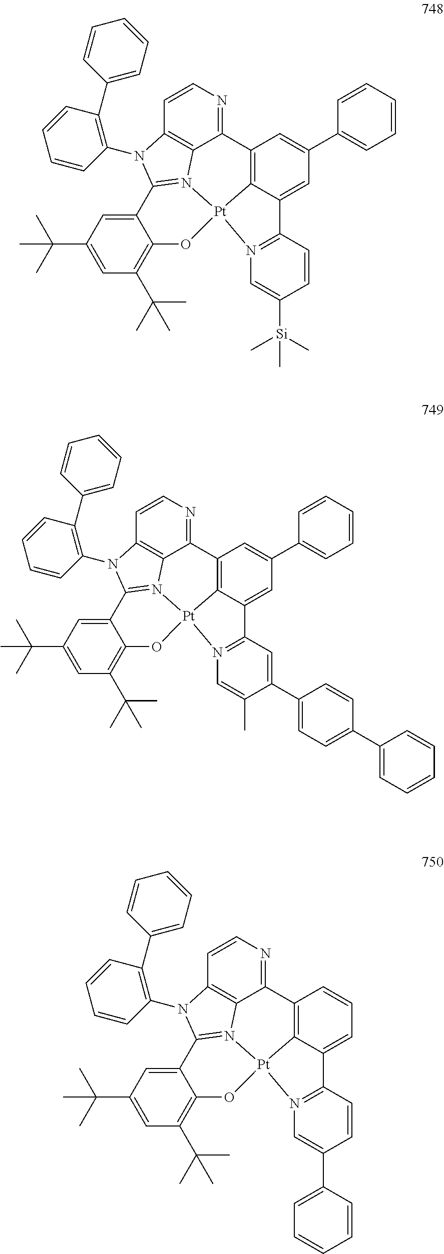

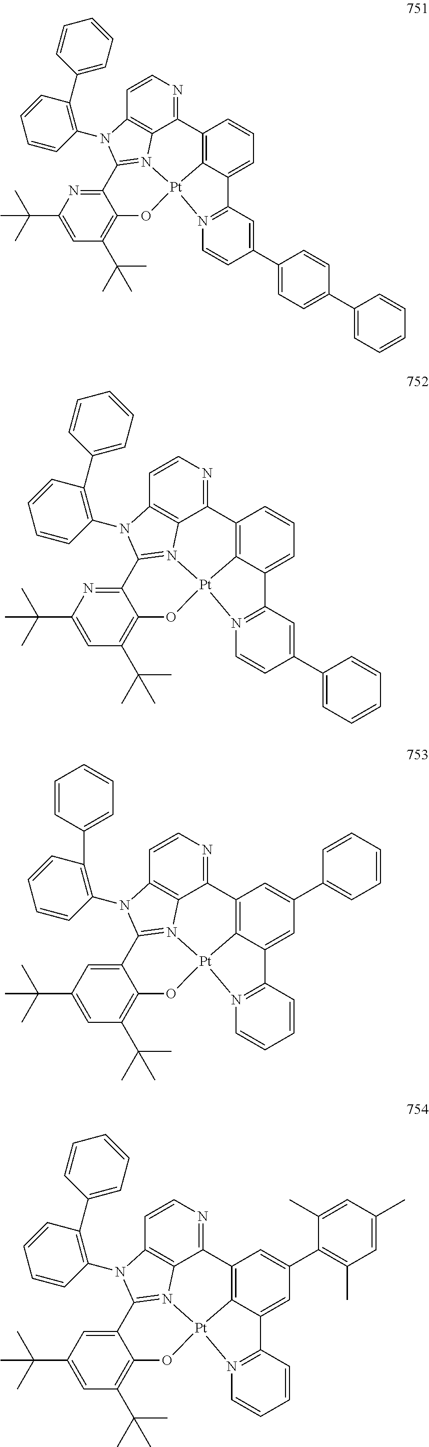

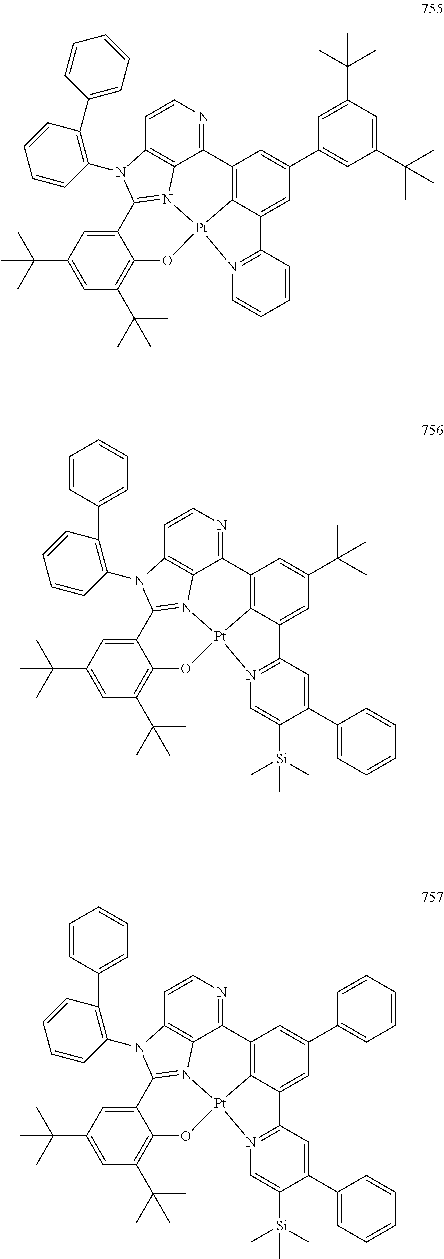

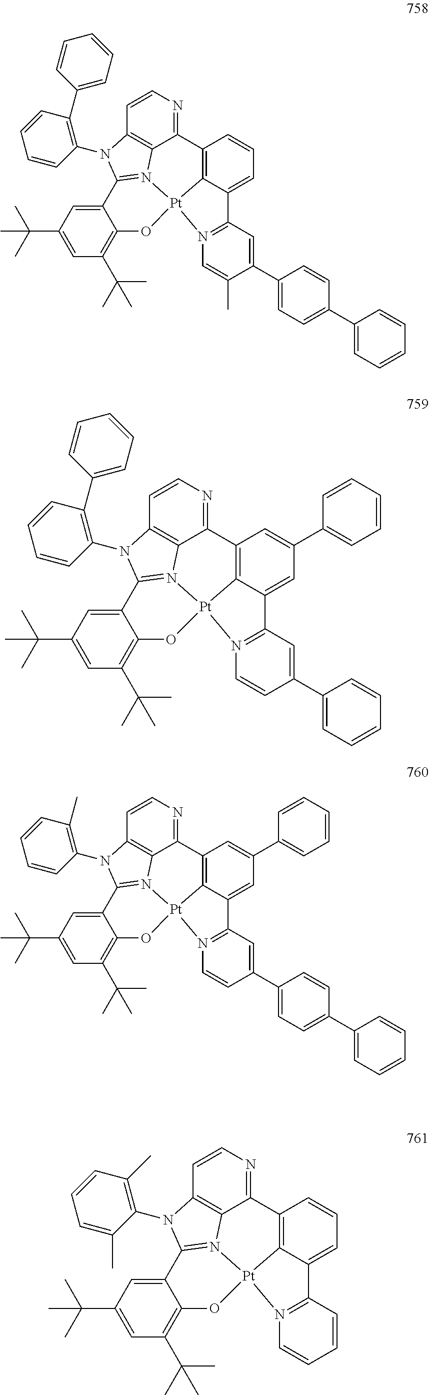

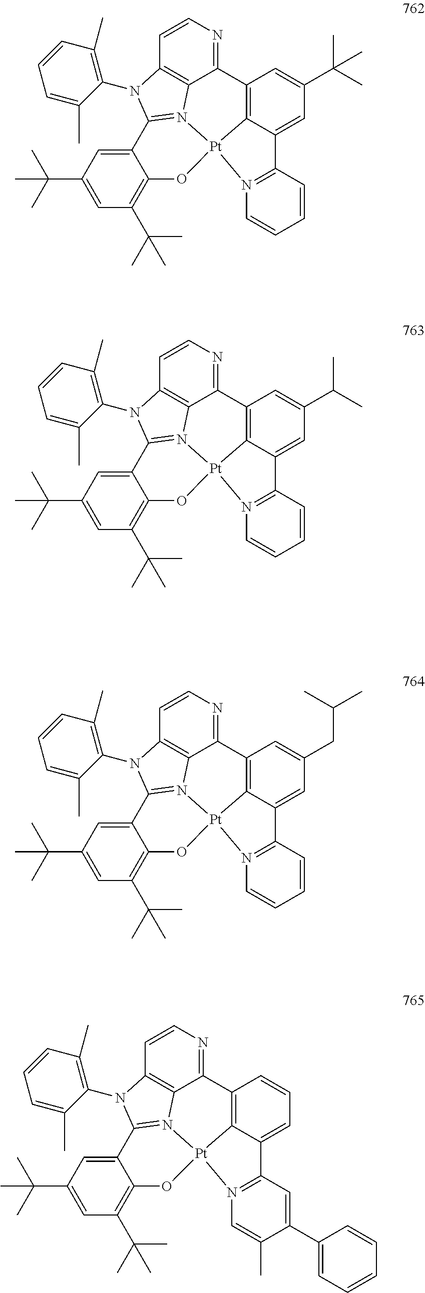

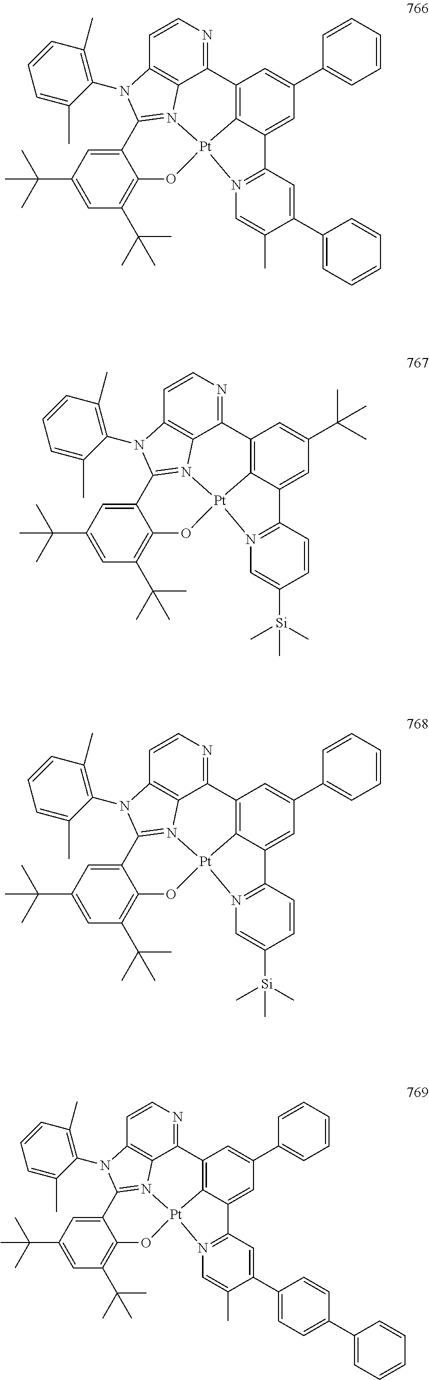

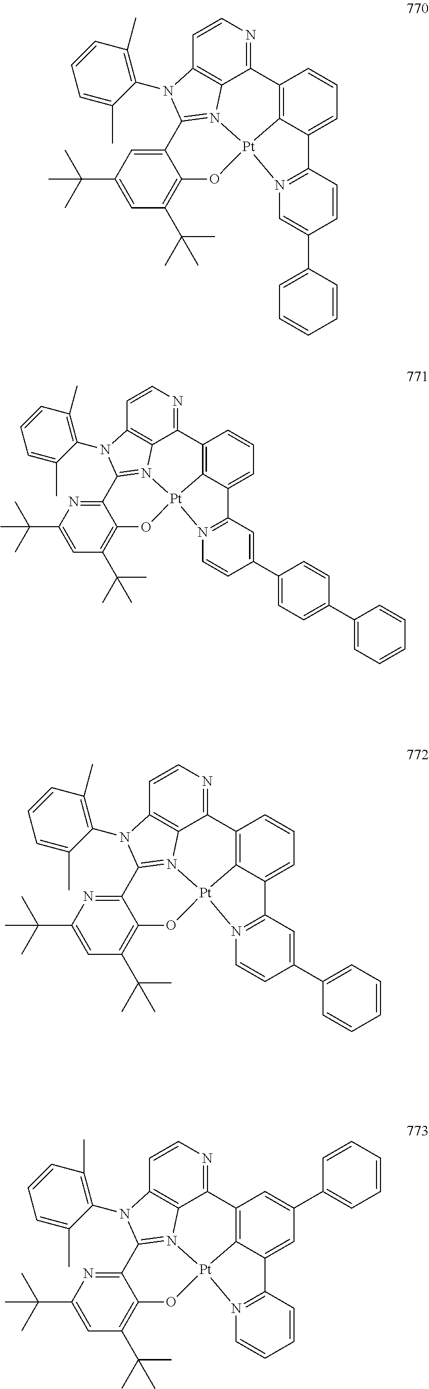

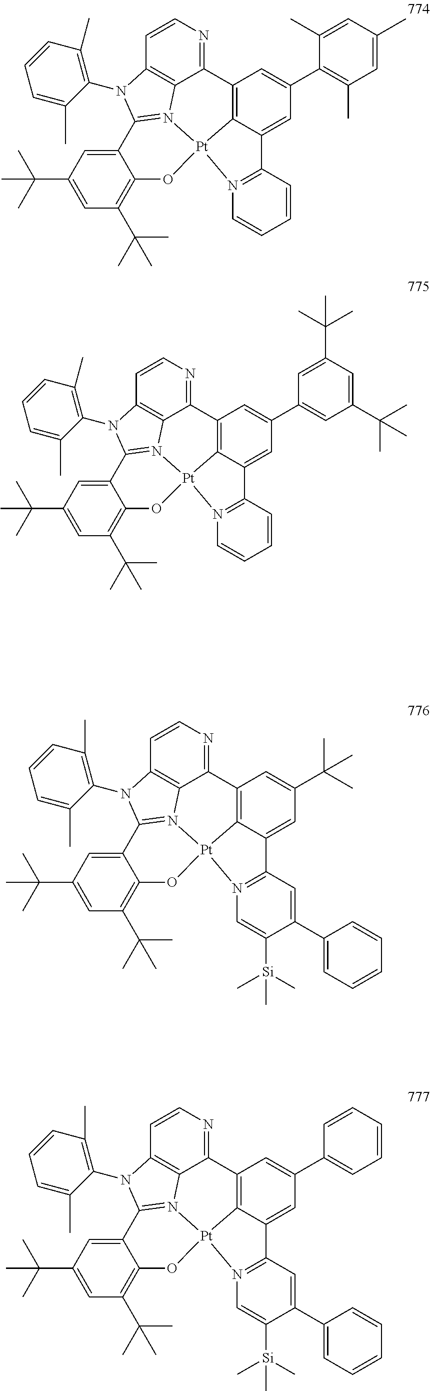

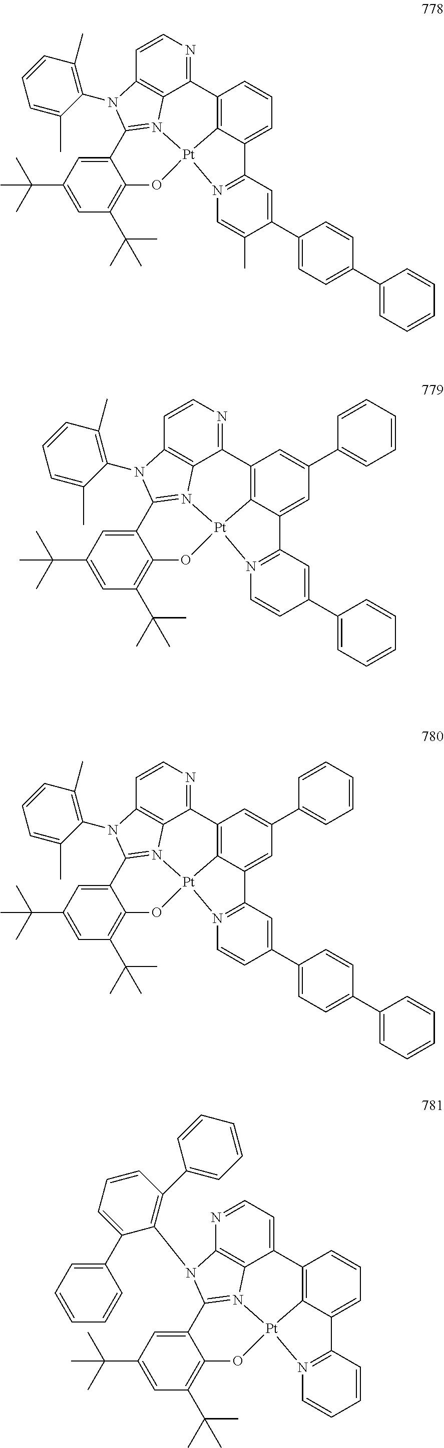

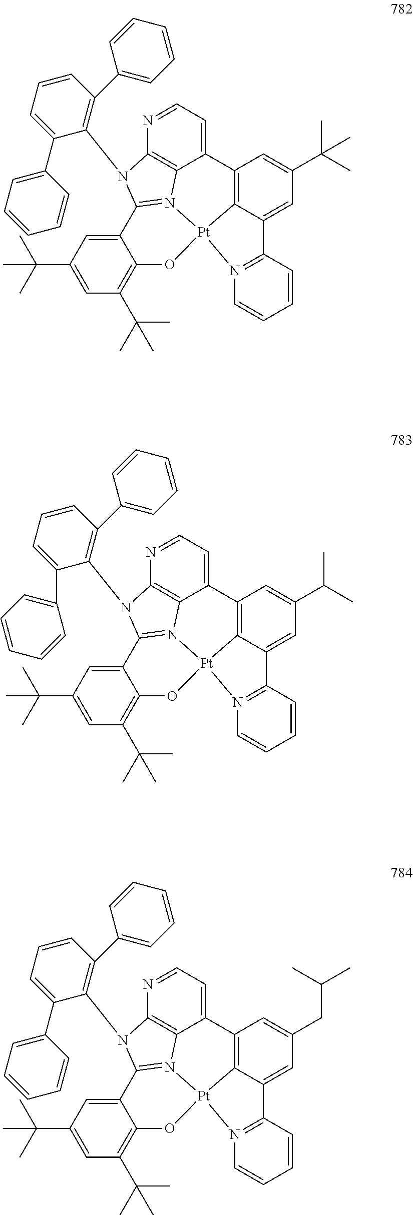

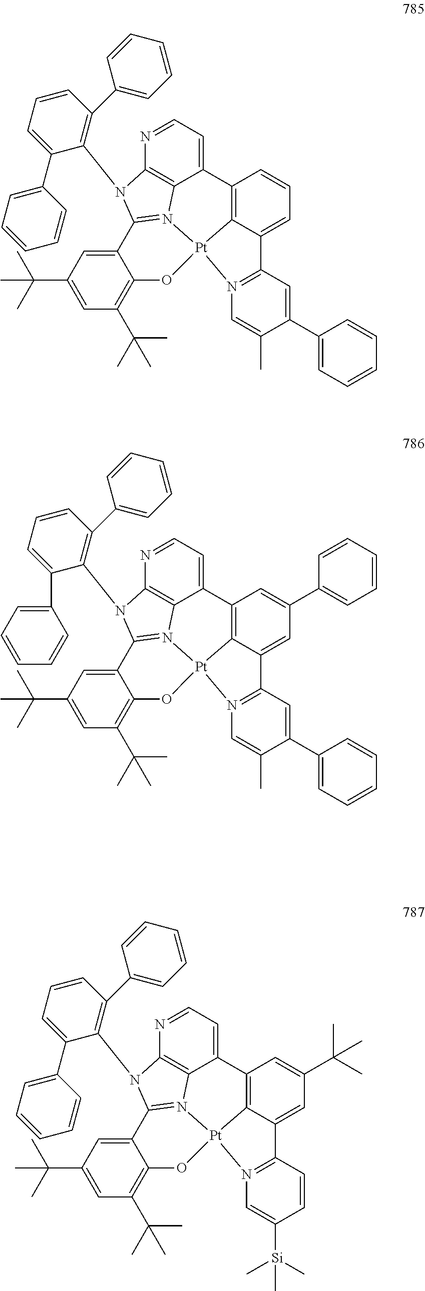

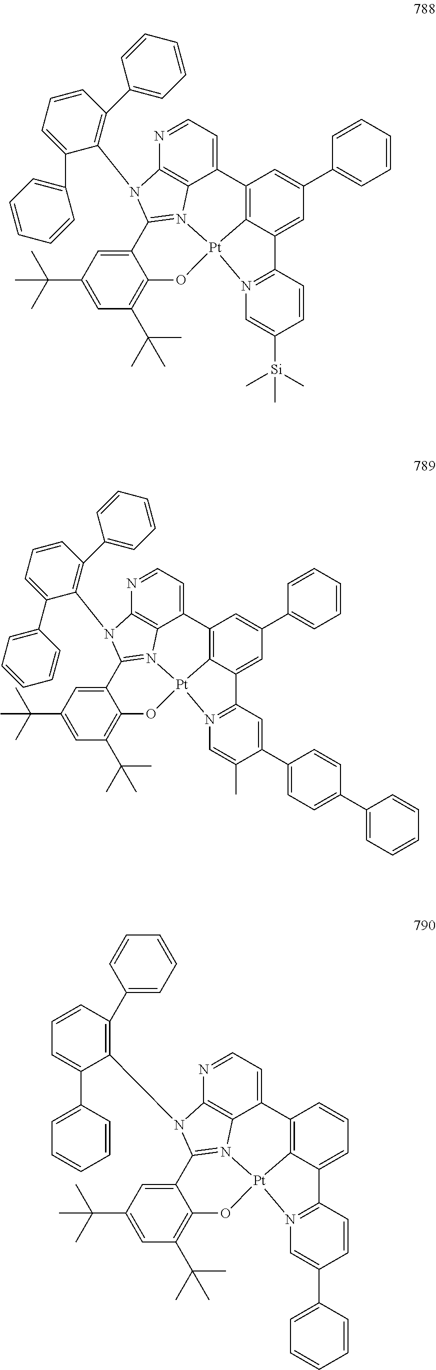

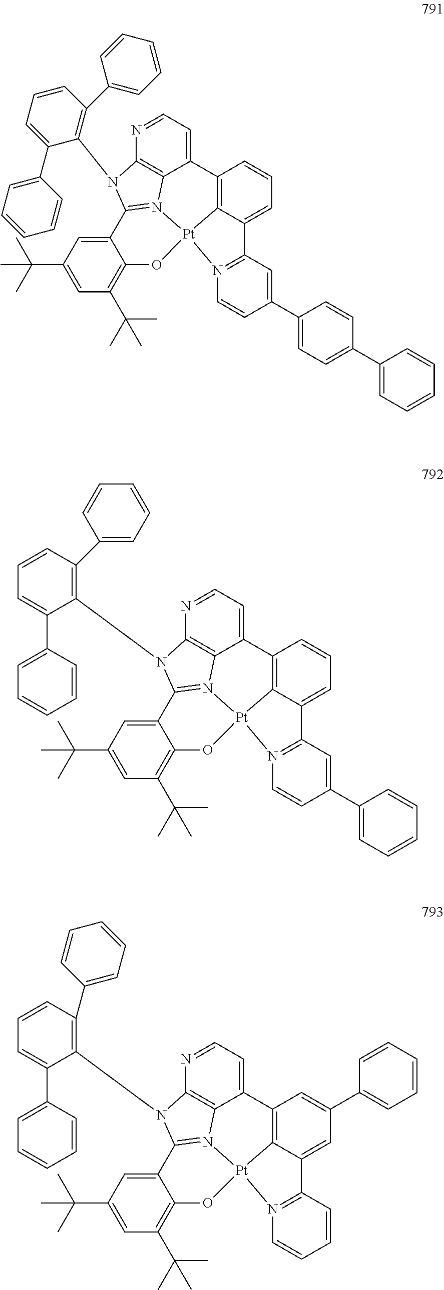

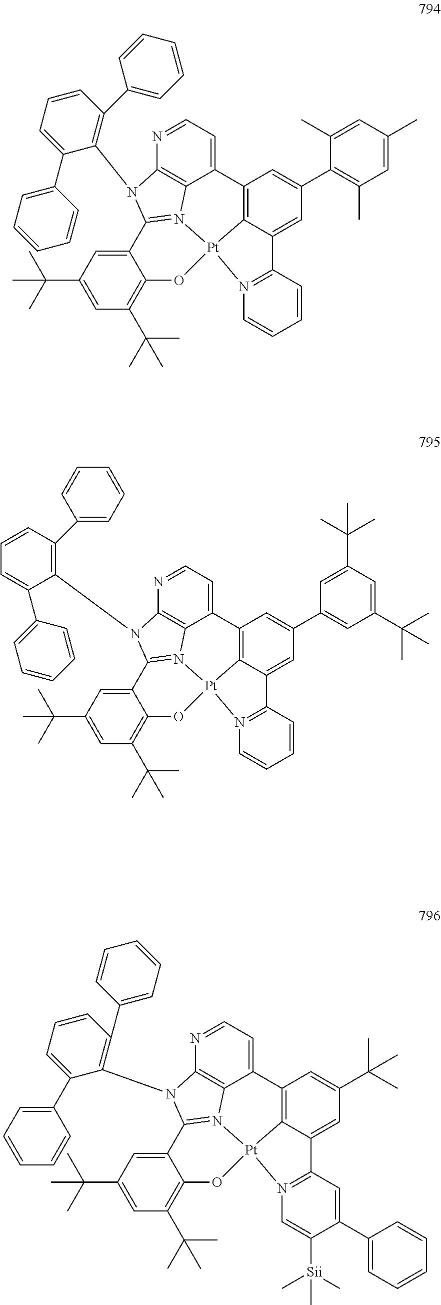

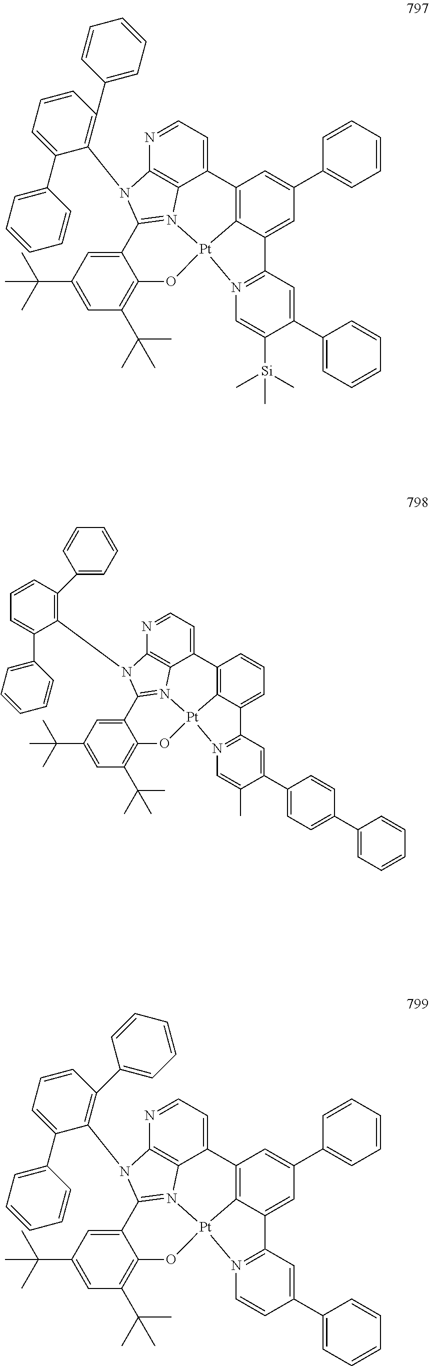

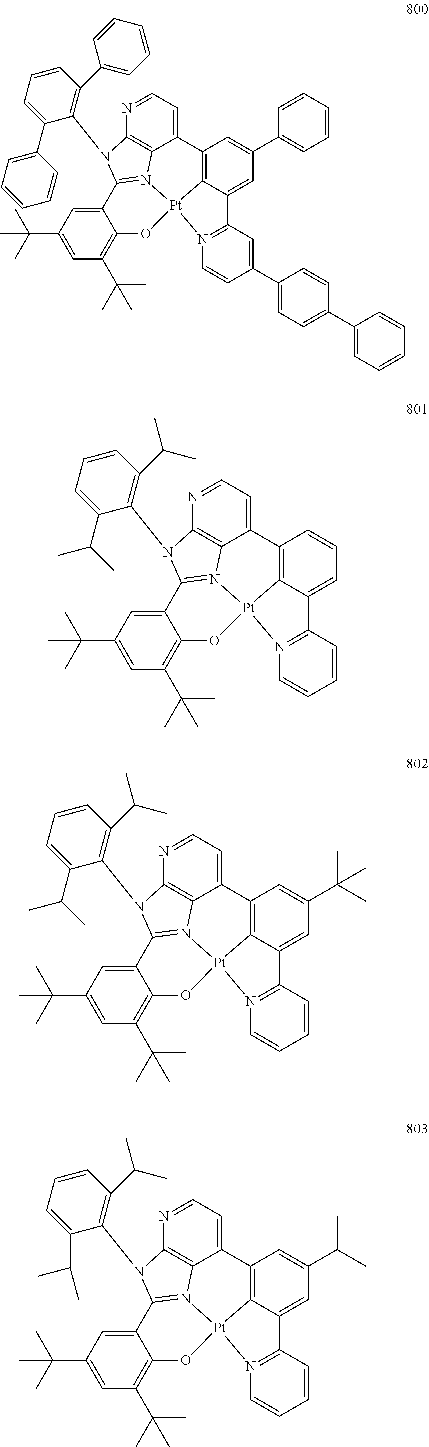

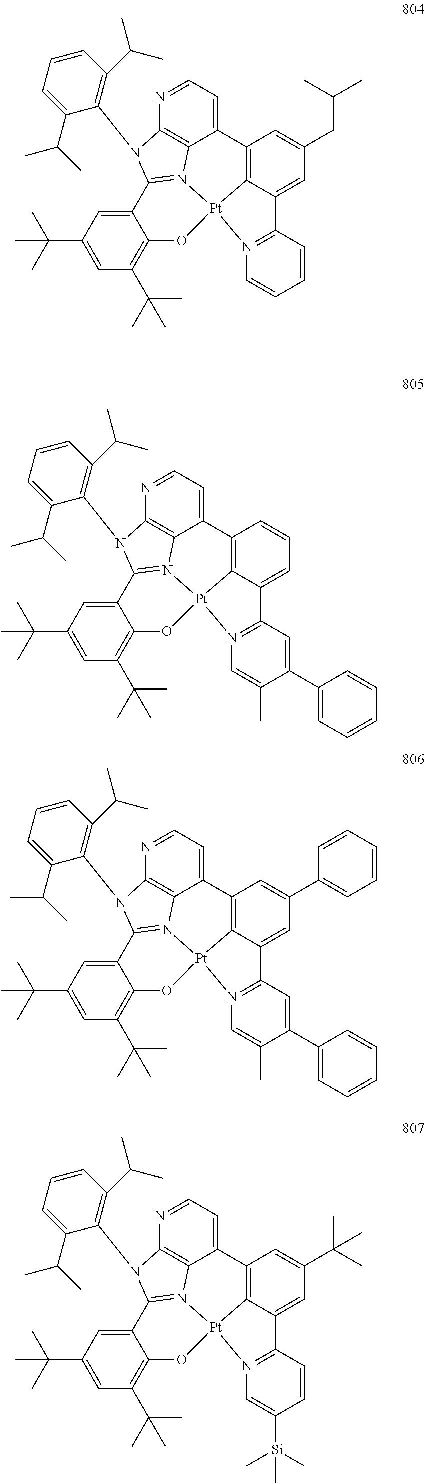

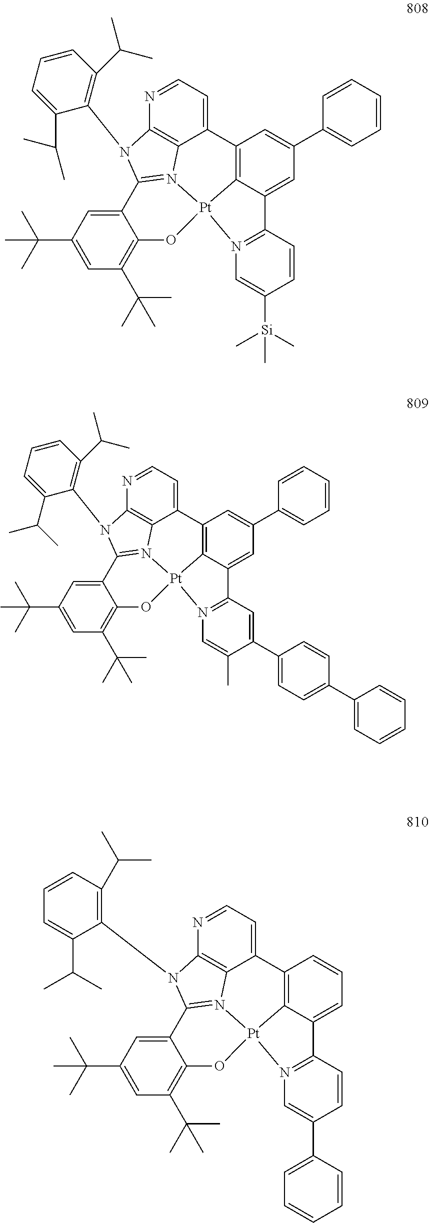

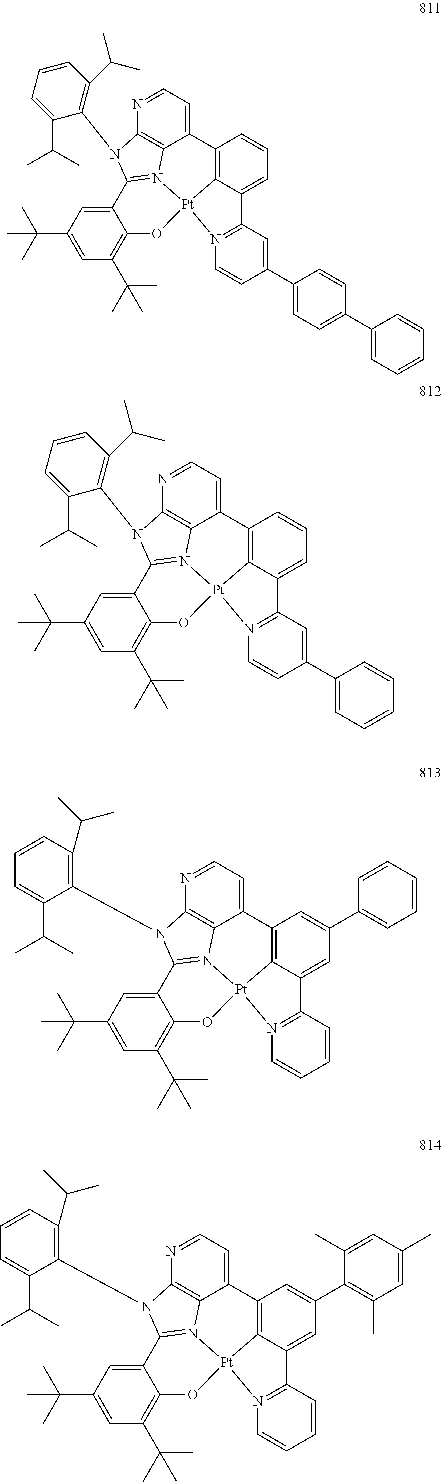

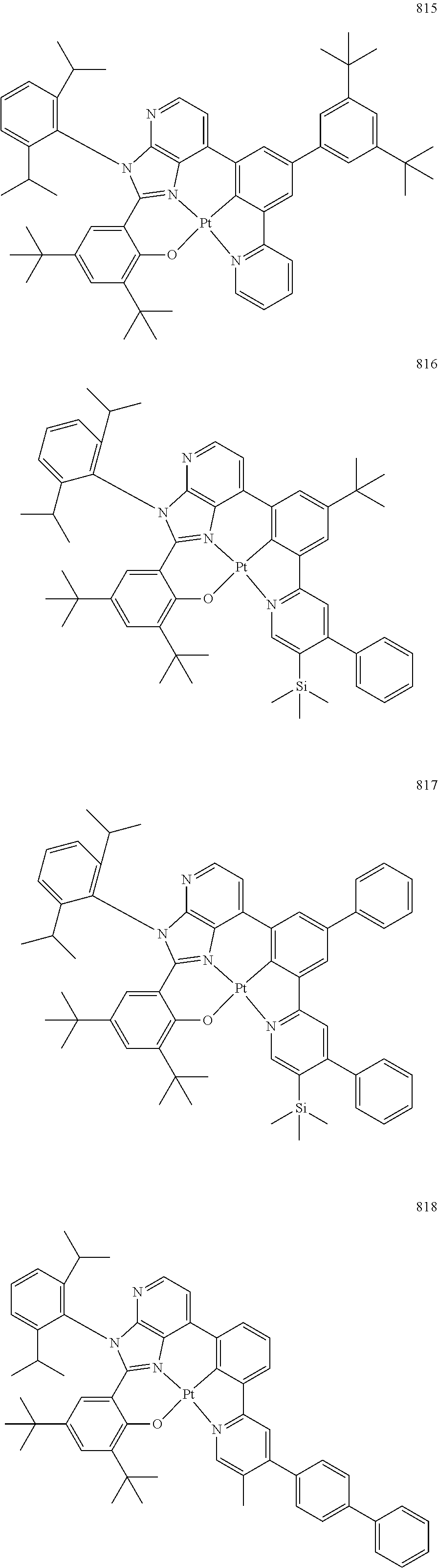

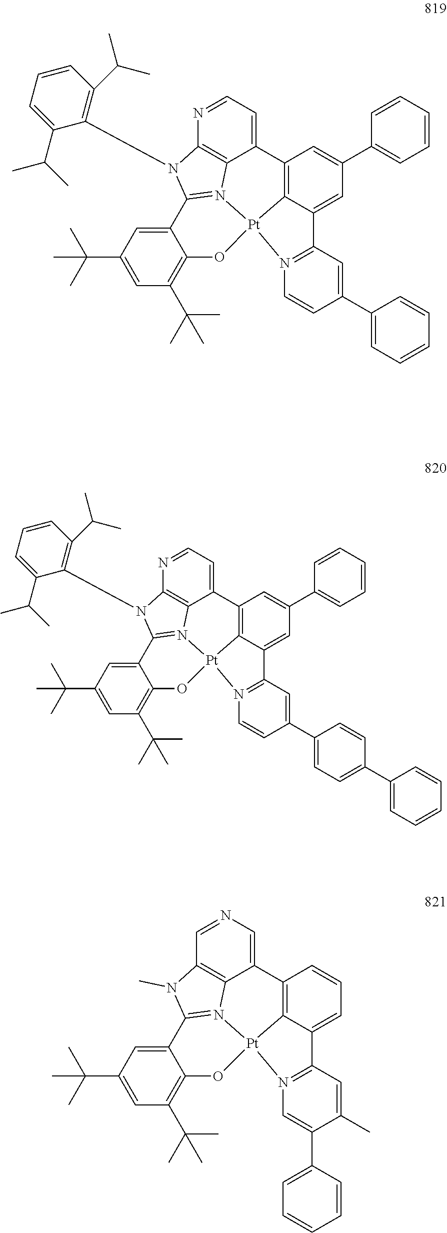

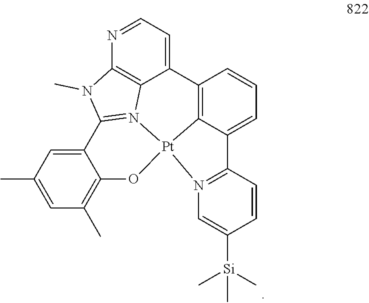

An electronic device, for example, an organic light-emitting device which includes the organometallic compound represented by Formula 1, may have improved characteristics in terms of a driving voltage, a current density, luminance, current efficiency, power efficiency, color purity, a roll-off ratio, and/or a lifespan.

For example, a highest occupied molecular orbital (HOMO) energy level, a lowest unoccupied molecular orbital (LUMO) energy level, a singlet (Si) energy level, a triplet (T₁) energy level, and a maximum emission wavelength of each of Compounds 2, 153, 821, 822, 261, and 241 were evaluated by a density functional theory (DFT) method of a Gaussian program (the structure was optimized at a B3LYP, 6-31G(d,p) level). Evaluation results are shown in Table 1.

TABLE 1

| | | | S₁ | T₁ | Maximum |
|---|---|---|---|---|---|
| | | | energy | energy | emission |
| Compound | HOMO | LUMO | level | level | wavelength |
| No. | (eV) | (eV) | (eV) | (eV) | (nm) |
| 2 | −5.032 | −1.549 | 2.893 | 2.555 | 485 |
| 153 | −5.028 | −1.652 | 2.796 | 2.497 | 497 |
| 821 | −4.903 | −1.691 | 2.716 | 2.410 | 515 |
| 822 | −4.828 | −1.717 | 2.620 | 2.318 | 535 |
| 261 | −4.982 | −1.587 | 2.857 | 2.430 | 510 |
| 241 | −5.041 | −1.612 | 2.886 | 2.472 | 502 |

From Table 1, it has been determined that the organometallic compound represented by Formula 1 has electrical characteristics that are suitable for use in an electronic device, for example, for use as a dopant for an organic light-emitting device.

Synthesis methods of the organometallic compound represented by Formula 1 may be recognizable by those of ordinary skill in the art by referring to Synthesis Examples provided below.

The organometallic compound represented by Formula 1 may be suitable for use in an organic layer of an organic light-emitting device, for example, for use as a dopant of an emission layer in the organic layer. Thus, another aspect of the present disclosure provides an organic light-emitting device including:
a first electrode;
a second electrode; and
an organic layer disposed between the first electrode and the second electrode,
wherein the organic layer includes an emission layer and at least one organometallic compound represented by Formula 1.

The organic light-emitting device may have, due to the inclusion of the organic layer including the organometallic compound represented by Formula 1, a low driving voltage, high luminescent efficiency, high power efficiency, high quantum efficiency, a long lifespan, a low roll-off ratio, and excellent color purity.

The organometallic compound of Formula 1 may be used between a pair of electrodes of an organic light-emitting device. For example, the organometallic compound represented by Formula 1 may be included in the emission layer. In this regard, the organometallic compound may act as a dopant, and the emission layer may further include a host (that is, an amount of the organometallic compound represented by Formula 1 is smaller than an amount of the host).

The expression “(an organic layer) includes at least one of organometallic compounds” as used herein may include an embodiment in which “(an organic layer) includes identical organometallic compounds represented by Formula 1” and an embodiment in which “(an organic layer) includes two or more different organometallic compounds represented by Formula 1”.

For example, the organic layer may include, as the organometallic compound, only Compound 1. In this regard, Compound 1 may be included in an emission layer of the organic light-emitting device. In one or more embodiments, the organic layer may include, as the organometallic compound, Compound 1 and Compound 2. In this regard, Compound 1 and Compound 2 may be included in an identical layer (for example, Compound 1 and Compound 2 all may be included in an emission layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

In an embodiment, in the organic light-emitting device, the first electrode is an anode, the second electrode is a cathode, and the organic layer further includes a hole transport region disposed between the first electrode and the emission layer and an electron transport region disposed between the emission layer and the second electrode, wherein the hole transport region includes a hole injection layer, a hole transport layer, an electron blocking layer, or any combination thereof, and wherein the electron transport region includes a hole blocking layer, an electron transport layer, an electron injection layer, or any combination thereof.

The term “organic layer” as used herein refers to a single layer and/or a plurality of layers disposed between the first electrode and the second electrode of the organic light-emitting device. The “organic layer” may include, in addition to an organic compound, an organometallic complex including metal.

The FIGURE is a schematic view of an organic light-emitting device 10 according to an embodiment. Hereinafter, the structure of an organic light-emitting device according to an embodiment and a method of manufacturing an organic light-emitting device according to an embodiment will be described in connection with the FIGURE. The organic light-emitting device 10 includes a first electrode 11, an organic layer 15, and a second electrode 19, which are sequentially stacked.

A substrate may be additionally disposed under the first electrode 11 or above the second electrode 19. For use as the substrate, any substrate that is used in general organic light-emitting devices may be used, and the substrate may be a glass substrate or a transparent plastic substrate, each having excellent mechanical strength, thermal stability, transparency, surface smoothness, ease of handling, and water resistance.

The first electrode 11 may be formed by depositing or sputtering a material for forming the first electrode 11 on the substrate. The first electrode 11 may be an anode. The material for forming the first electrode 11 may be selected from materials with a high work function to facilitate hole injection. The first electrode 11 may be a reflective electrode, a semi-transmissive electrode, or a transmissive electrode. The material for forming the first electrode may be, for example, indium tin oxide (ITO), indium zinc oxide (IZO), tin oxide (SnO₂), and zinc oxide (ZnO). In one or more embodiments, magnesium (Mg), aluminum (Al), aluminum-lithium (Al—Li), calcium (Ca), magnesium-indium (Mg—In), or magnesium-silver (Mg—Ag) may be used as the material for forming the first electrode.

The first electrode 11 may have a single-layered structure or a multi-layered structure including two or more layers. For example, the first electrode 11 may have a three-layered structure of ITO/Ag/ITO, but the structure of the first electrode 110 is not limited thereto.

The organic layer 15 is disposed on the first electrode 11.

The organic layer 15 may include a hole transport region, an emission layer, and an electron transport region.

The hole transport region may be disposed between the first electrode 11 and the emission layer.

The hole transport region may include a hole injection layer, a hole transport layer, an electron blocking layer, a buffer layer, or any combination thereof.

The hole transport region may include only either a hole injection layer or a hole transport layer. In one or more embodiments, the hole transport region may have a hole injection layer/hole transport layer structure or a hole injection layer/hole transport layer/electron blocking layer structure, which are sequentially stacked in this stated order from the first electrode 11.

A hole injection layer may be formed on the first electrode 11 by using one or more suitable methods selected from vacuum deposition, spin coating, casting, or Langmuir-Blodgett (LB) deposition.

When a hole injection layer is formed by vacuum deposition, the deposition conditions may vary depending on a material that is used to form the hole injection layer, and the structure and thermal characteristics of the hole injection layer. For example, the deposition conditions may include a deposition temperature of about 100²C to about 500° C., a vacuum pressure of about 10⁻⁸ torr to about 10⁻³ torr, and a deposition rate of about 0.01 Angstroms per second (A/sec) to about 100 Å/sec. However, the deposition conditions are not limited thereto.

When the hole injection layer is formed using spin coating, coating conditions may vary depending on the material used to form the hole injection layer, and the structure and thermal properties of the hole injection layer. For example, a coating speed may be from about 2,000 revolutions per minute (rpm) to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include at least one selected from m-MTDATA, TDATA, 2-TNATA, NPB, β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzene sulfonic acid (PANI/DBSA), poly(3,4-ethylenedioxythiophene)/poly(4-styrene sulfonate) (PEDOT/PSS), polyaniline/camphor sulfonic acid (PANI/CSA), polyaniline/poly(4-styrene sulfonate) (PANI/PSS), a compound represented by Formula 201 below, and a compound represented by Formula 202 below:

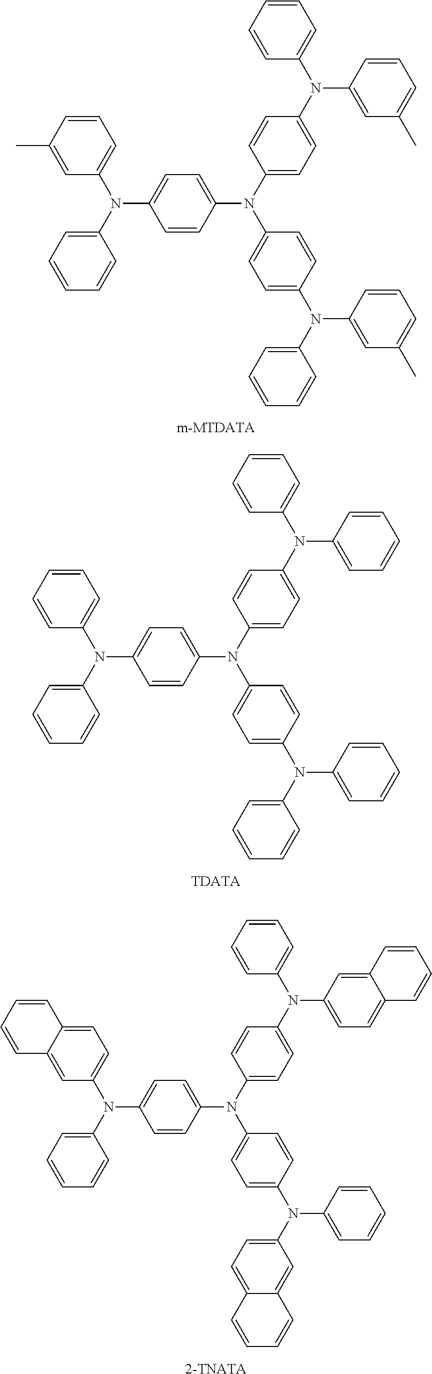

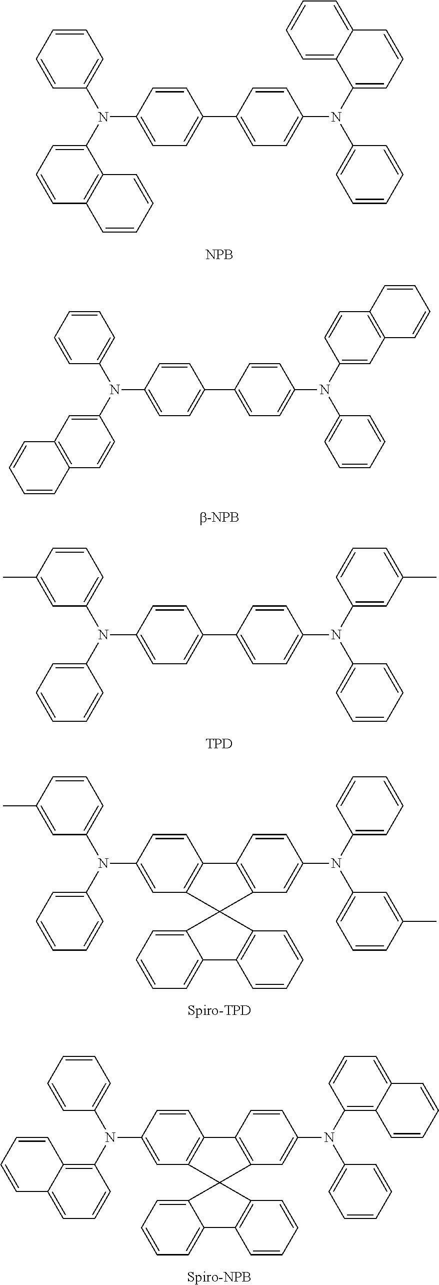

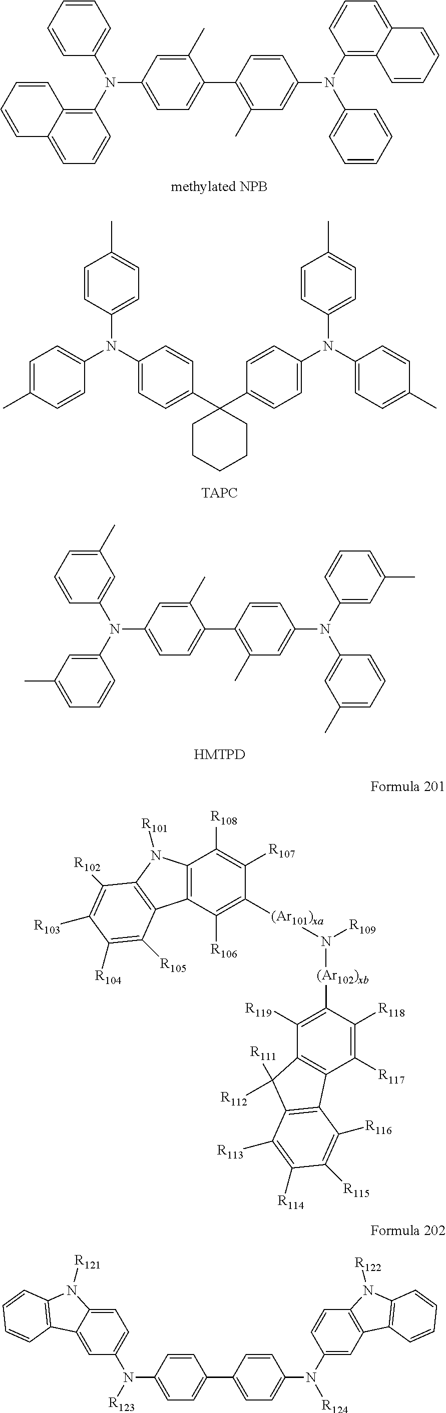

Ar₁₀₁ and Ar₁₀₂ in Formula 201 may each independently be selected from:
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group; and
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

In Formula 201, xa and xb may each independently be an integer from 0 to 5, or 0, 1, or 2. For example, xa is 1 and xb is 0, but xa and xb are not limited thereto.

R₁₀₁ to R₁₀₈, R₁₁₁ to R₁₁₉, and R₁₂₁ to R₁₂₄ in Formulae 201 and 202 may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, a pentyl group, a hexyl group, and so on), or a C₁-C₁₀ alkoxy group (for example, a methoxy group, an ethoxy group, a propoxy group, a butoxy group, a pentoxy group, and so on);

a C₁-C₁₀ alkyl group or a C₁-C₁₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group, but embodiments of the present disclosure are not limited thereto.

R₁₀₉ in Formula 201 may be selected from:

a phenyl group, a naphthyl group, an anthracenyl group, and a pyridinyl group; and

a phenyl group, a naphthyl group, an anthracenyl group, and a pyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, and a pyridinyl group.

According to an embodiment, the compound represented by Formula 201 may be represented by Formula 201A, but embodiments of the present disclosure are not limited thereto:

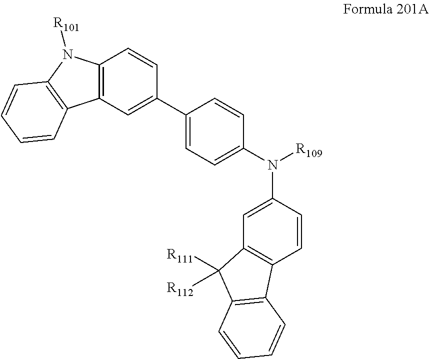

R₁₀₁, R₁₁₁, R₁₁₂, and R₁₀₉ in Formula 201A may be understood by referring to the description provided herein.

For example, the compound represented by Formula 201, and the compound represented by Formula 202 may include compounds HT1 to HT20 illustrated below, but are not limited thereto.

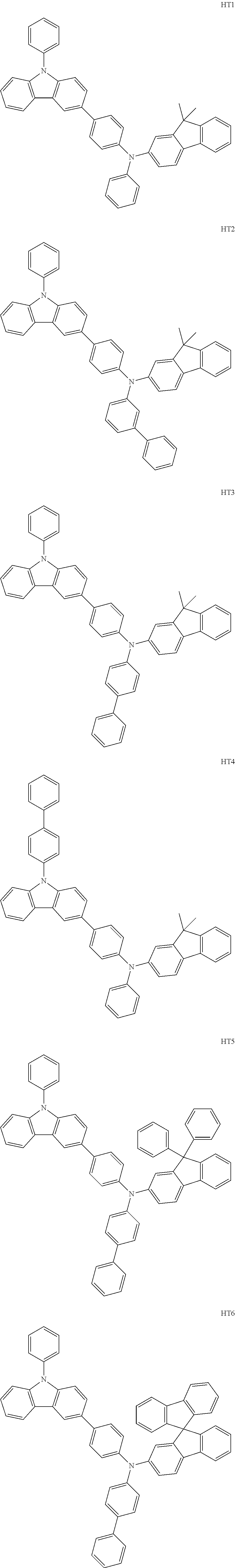

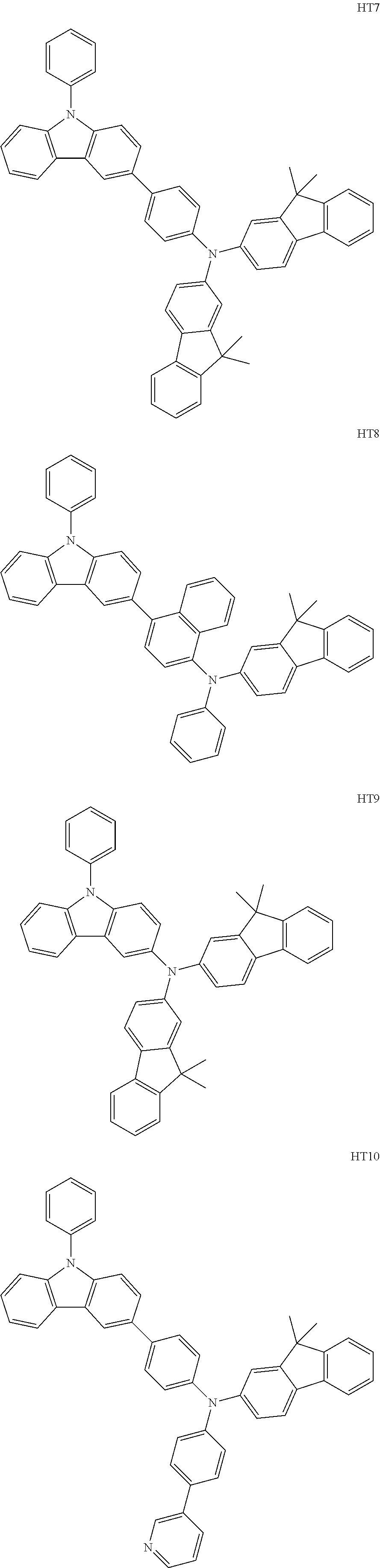 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

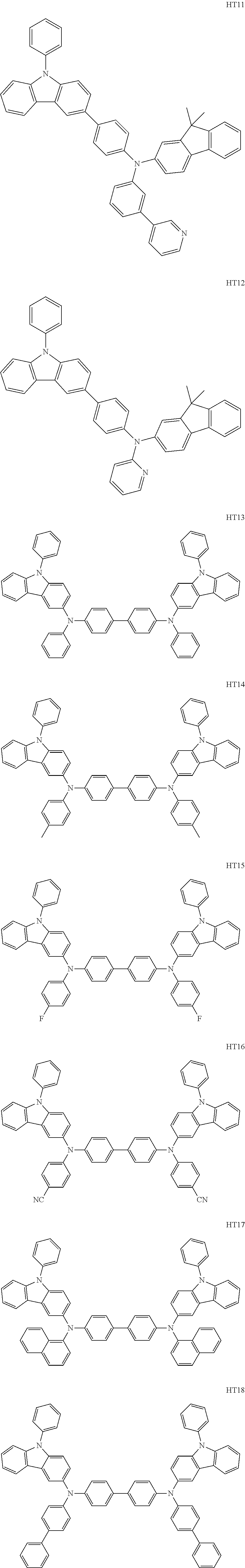

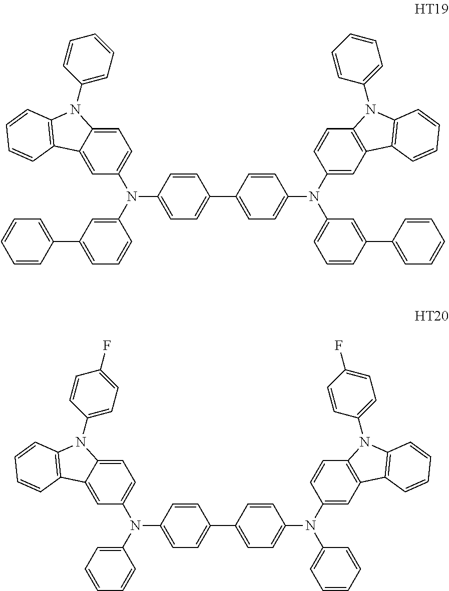 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes at least one of a hole injection layer and a hole transport layer, the thickness of the hole injection layer may be in a range of about 100 Å to about 10,000 Å, and for example, about 100 Å to about 1,000 Å, and the thickness of the hole transport layer may be in a range of about 50 Å to about 2,000 Å, and for example, about 100 Å to about 1,500 Å. While not wishing to be bound by theory, it is understood that when the thicknesses of the hole transport region, the hole injection layer and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenium oxide; and a cyano group-containing compound, such as Compound HT-D1 below, but are not limited thereto.

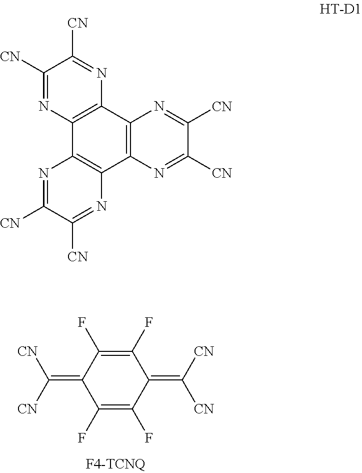

The hole transport region may include a buffer layer.

Also, the buffer layer may compensate for an optical resonance distance depending on a wavelength of light emitted from the emission layer, and thus, efficiency of a formed organic light-emitting device may be improved.

Then, an emission layer may be formed on the hole transport region by vacuum deposition, spin coating, casting, LB deposition, or the like. When the emission layer is formed by vacuum deposition or spin coating, the deposition or coating conditions may be similar to those applied to form the hole injection layer although the deposition or coating conditions may vary according to the compound that is used to form the emission layer.

Meanwhile, when the hole transport region includes an electron blocking layer, a material for the electron blocking layer may be selected from materials for the hole transport region described above and materials for a host to be explained later. However, the material for the electron blocking layer is not limited thereto. For example, when the hole transport region includes an electron blocking layer, a material for the electron blocking layer may be mCP, which will be explained later.

The emission layer may include a host and a dopant, and the dopant may include the organometallic compound represented by Formula 1.

The host may include at least one selected from TPBi, TBADN, ADN (also referred to as “DNA”), CBP, CDBP, TCP, mCP, Compound H50, and Compound H51:

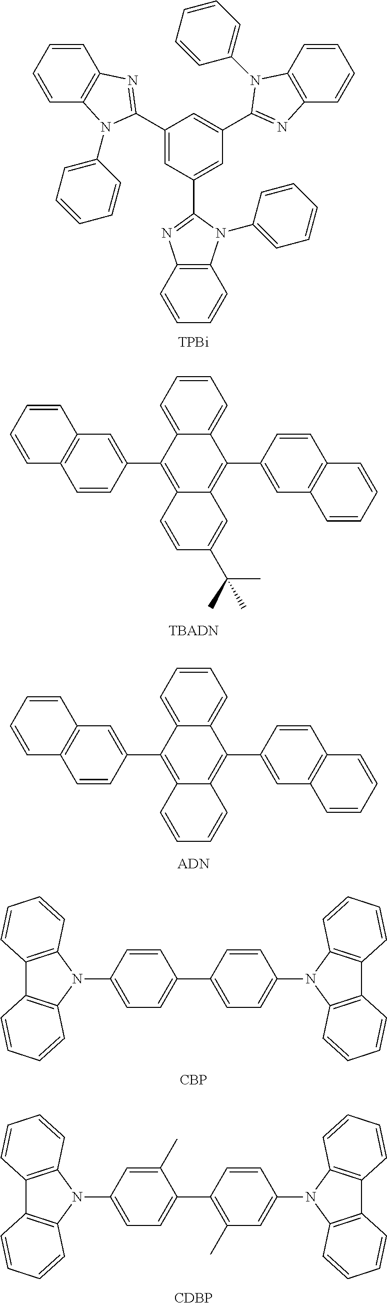

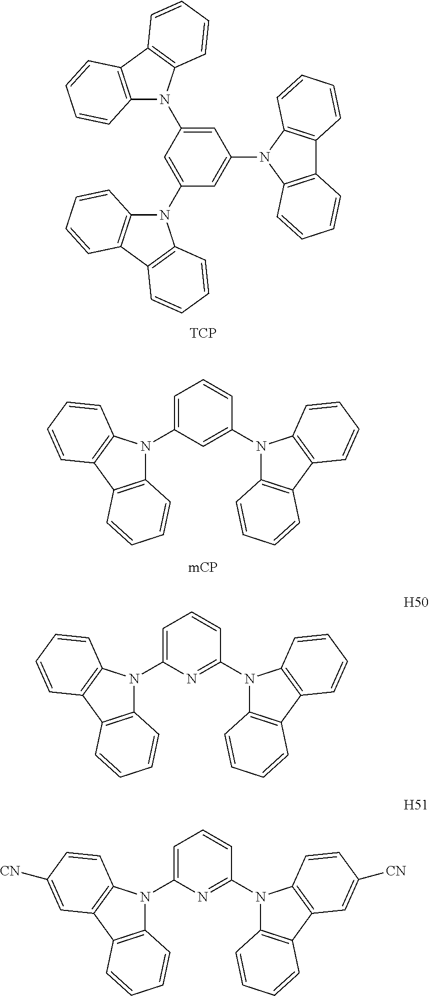

In one or more embodiments, the host may further include a compound represented by Formula 301 below.

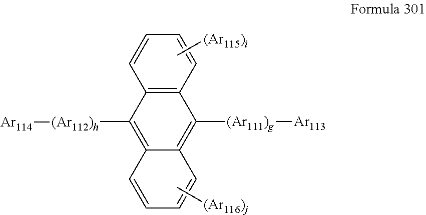

Ar₁₁₁ and Ar₁₁₂ in Formula 301 may each independently be selected from:

a phenylene group, a naphthylene group, a phenanthrenylene group, and a pyrenylene group; and

a phenylene group, a naphthylene group, a phenanthrenylene group, and a pyrenylene group, each substituted with at least one selected from a phenyl group, a naphthyl group, and an anthracenyl group.

Ar₁₁₃ to Ar₁₁₆ in Formula 301 may each independently be selected from:

a C₁-C₁₀ alkyl group, a phenyl group, a naphthyl group, a phenanthrenyl group, and a pyrenyl group; and

a phenyl group, a naphthyl group, a phenanthrenyl group, and a pyrenyl group, each substituted with at least one selected from a phenyl group, a naphthyl group, and an anthracenyl group.

g, h, i, and j in Formula 301 may each independently be an integer from 0 to 4, and may be, for example, 0, 1, or 2.

Ar₁₁₃ to Ar₁₁₆ in Formula 301 may each independently be selected from:

a C₁-C₁₀ alkyl group, the substituted with at least one selected from a phenyl group, a naphthyl group, and an anthracenyl group;

a phenyl group, a naphthyl group, an anthracenyl group, a pyrenyl, a phenanthrenyl group, and a fluorenyl group;

a phenyl group, a naphthyl group, an anthracenyl group, a pyrenyl group, a phenanthrenyl group, and a fluorenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a phenyl group, a naphthyl group, an anthracenyl group, a pyrenyl group, a phenanthrenyl group, and a fluorenyl group; and

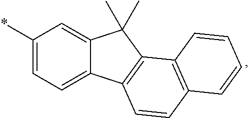 [SMILES: *c1ccc2c(c1)C(C)(C)c1c-2ccc2ccccc12]

but embodiments of the present disclosure are not limited thereto.

In one or more embodiments, the host may include a compound represented by Formula 302 below:

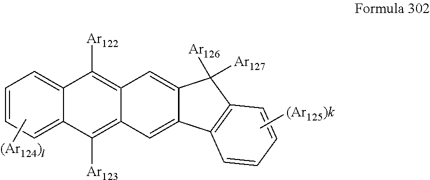

Ar₁₂₂ to Ar₁₂₅ in Formula 302 are the same as described in detail in connection with Ar₁₁₃ in Formula 301.

Ar₁₂₆ and Ar₁₂₇ in Formula 302 may each independently be a C₁-C₁₀ alkyl group (for example, a methyl group, an ethyl group, or a propyl group).

k and l in Formula 302 may each independently be an integer from 0 to 4. For example, k and l may be 0, 1, or 2.

The compound represented by Formula 301 and the compound represented by Formula 302 may include Compounds H1 to H42 illustrated below, but are not limited thereto.

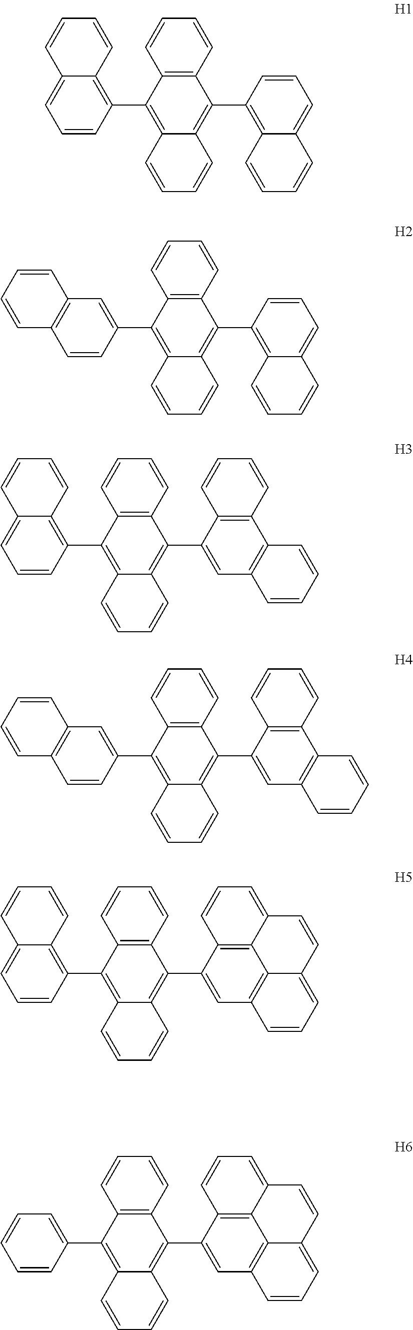

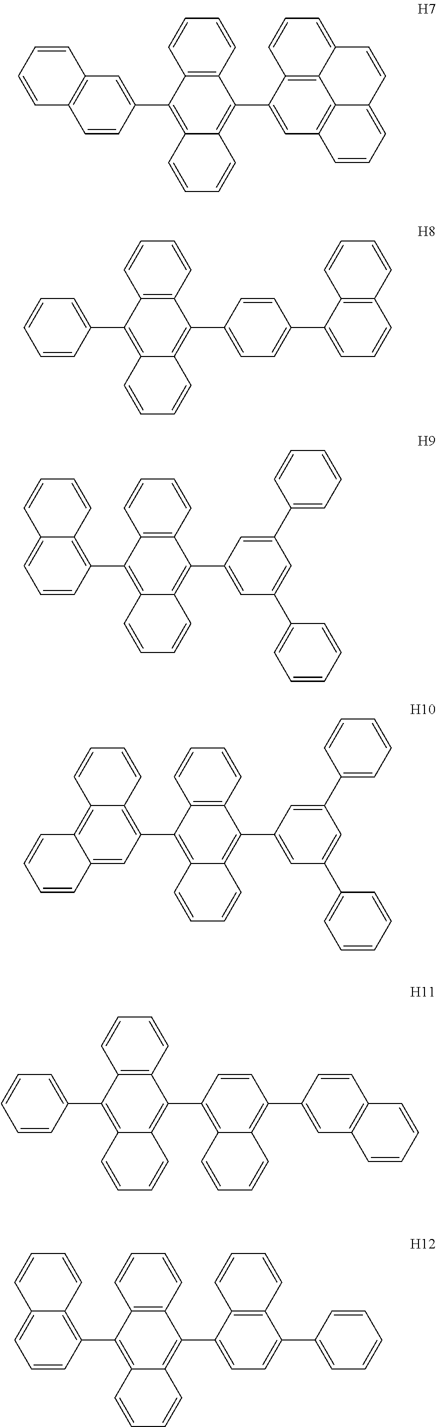

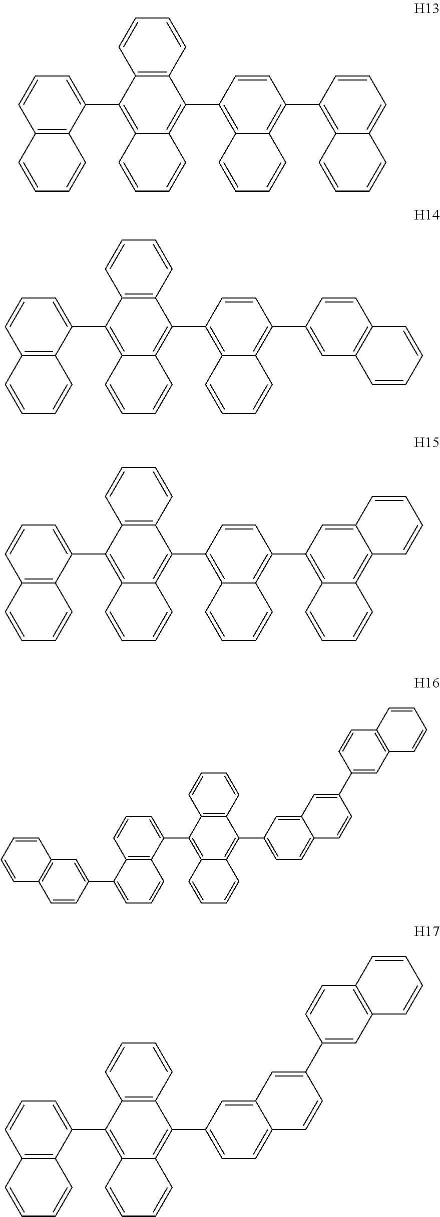

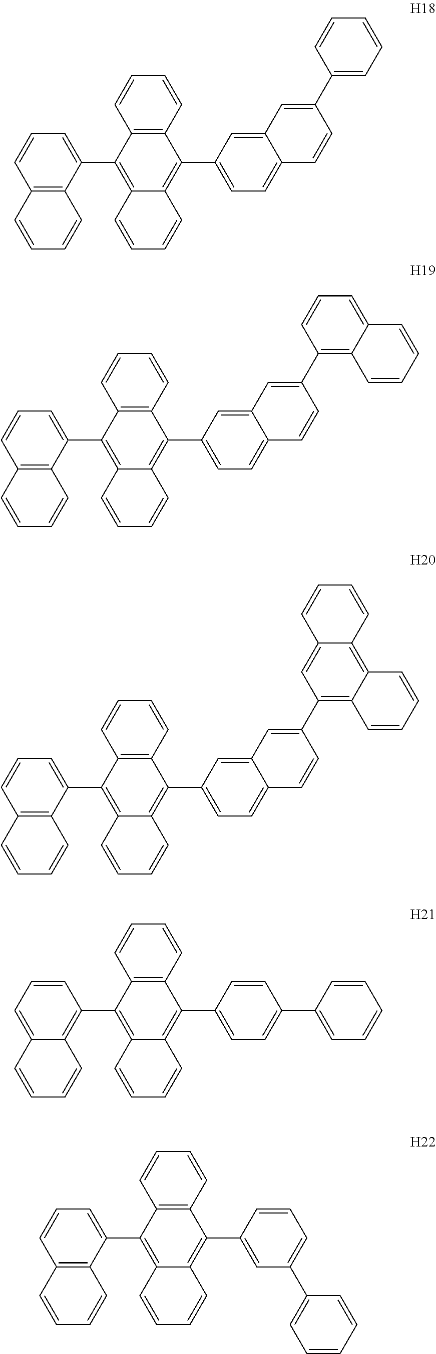

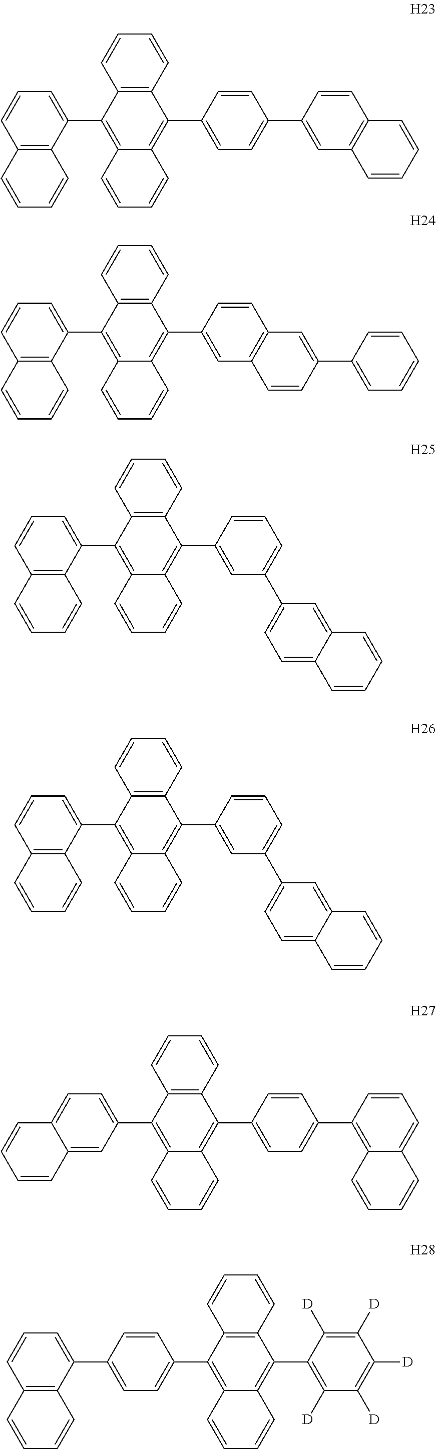

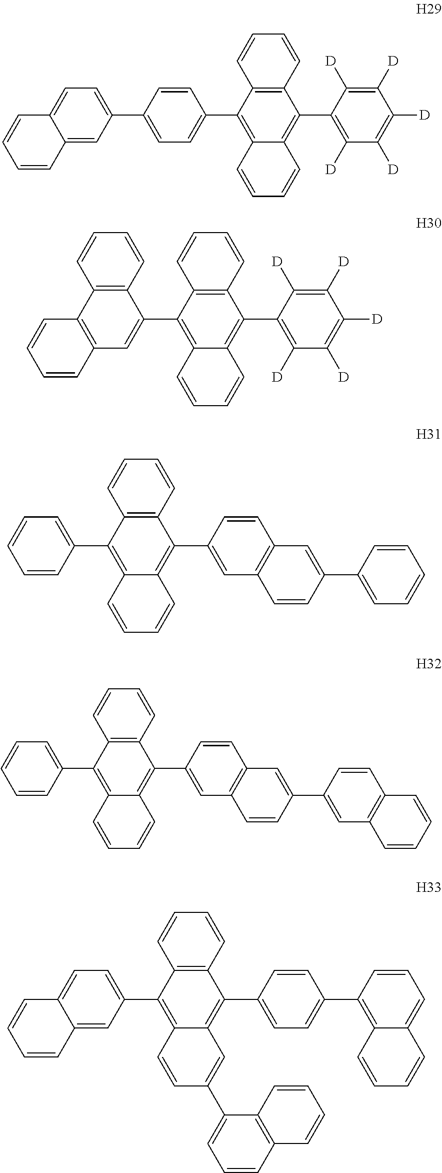

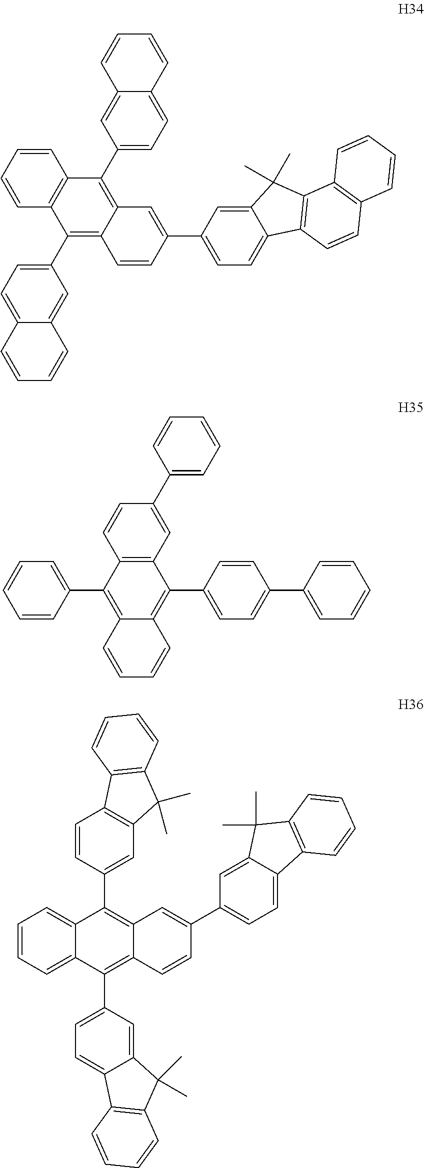

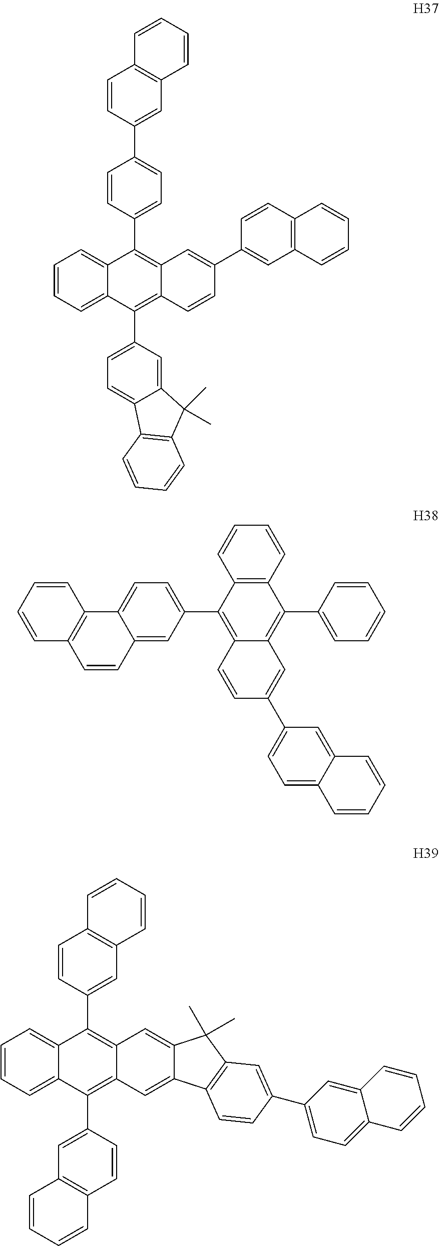

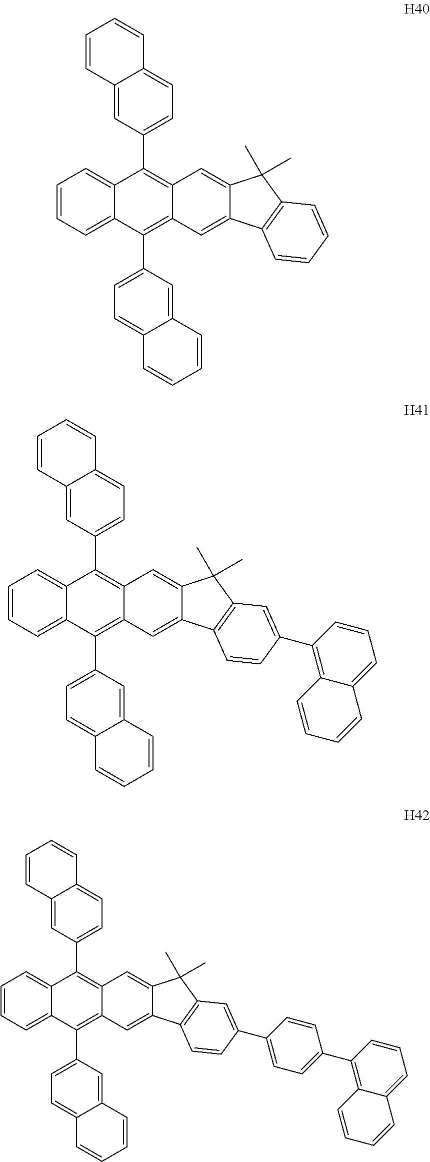

When the organic light-emitting device is a full-color organic light-emitting device, the emission layer may be patterned into a red emission layer, a green emission layer, and a blue emission layer. In one or more embodiments, due to a stack structure including a red emission layer, a green emission layer, and/or a blue emission layer, the emission layer may emit white light.

When the emission layer includes a host and a dopant, an amount of the dopant may be in a range of about 0.01 parts by weight to about 15 parts by weight based on 100 parts by weight of the host, but is not limited thereto.

A thickness of the emission layer may be in a range of about 100 Å to about 1,000 Å, for example, about 200 Å to about 600 Å. While not wishing to be bound by theory, it is understood that when the thickness of the emission layer is within this range, excellent light-emission characteristics may be obtained without a substantial increase in driving voltage.

Then, an electron transport region may be disposed on the emission layer.

The electron transport region may include a hole blocking layer, an electron transport layer, an electron injection layer, or any combination thereof.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP, Bphen, and BAlq but embodiments of the present disclosure are not limited thereto.

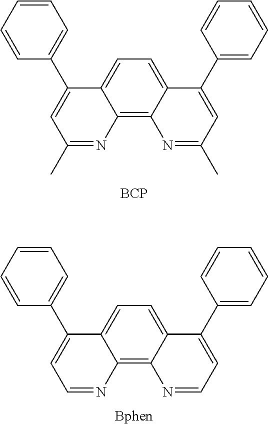

A thickness of the hole blocking layer may be in a range of about 20 Å to about 1,000 Å, for example, about 30 Å to about 300 Å. While not wishing to be bound by theory, it is understood that when the thickness of the hole blocking layer is within these ranges, the hole blocking layer may have improved hole blocking ability without a substantial increase in driving voltage.

The electron transport layer may further include at least one selected from BCP, Bphen, Alq₃, BAlq, TAZ, and NTAZ.

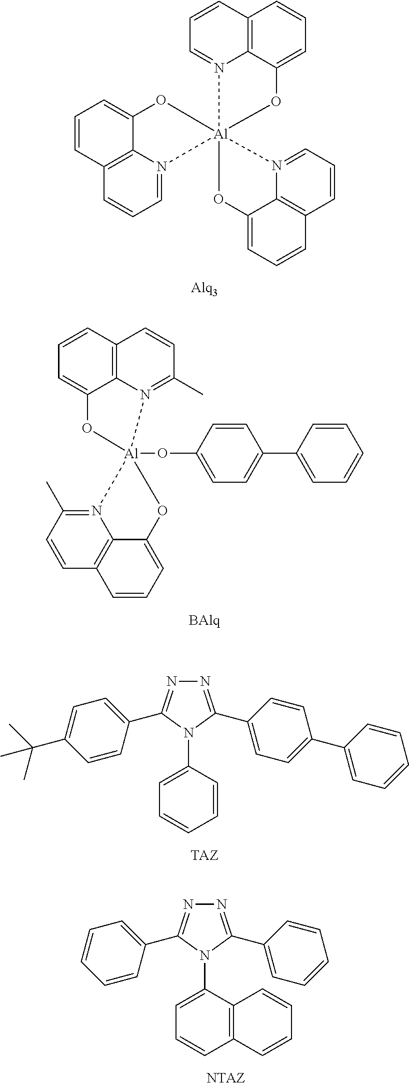

In one or more embodiments, the electron transport layer may include at least one of ET1 and ET2, but are not limited thereto:

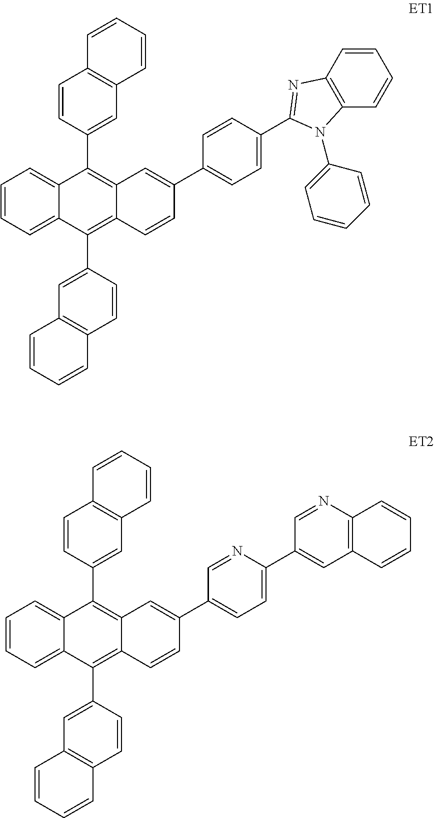 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1]

A thickness of the electron transport layer may be in a range of about 100 Å to about 1,000 Å, for example, about 150 Å to about 500 Å. While not wishing to be bound by theory, it is understood that when the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium quinolate, LiQ) or ET-D2.

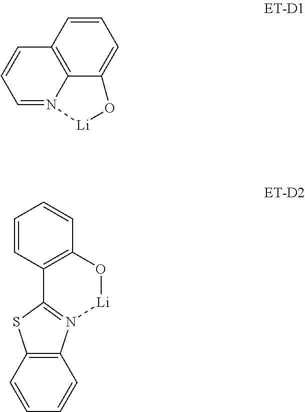

The electron transport region may also include an electron injection layer that facilitates injection of electrons from the second electrode 19.

The electron injection layer may include at least one selected from LiF, NaCl, CsF, Li₂O, and BaO.

A thickness of the electron injection layer may be in a range of about 1 Å to about 100 Å, for example, about 3 Å to about 90 Å. While not wishing to be bound by theory, it is understood that when the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be selected from metal, an alloy, an electrically conductive compound, and a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be used as a material for forming the second electrode 19. In one or more embodiments, to manufacture a top-emission type light-emitting device, a transmissive electrode formed using ITO or IZO may be used as the second electrode 19.

Hereinbefore, the organic light-emitting device has been described with reference to the FIGURE, but embodiments of the present disclosure are not limited thereto.

Another aspect of the present disclosure provides a diagnostic composition including at least one organometallic compound represented by Formula 1.

The organometallic compound represented by Formula 1 provides high luminescent efficiency. Accordingly, a diagnostic composition including the organometallic compound may have high diagnosis efficiency.

The diagnostic composition may be used in various applications including a diagnosis kit, a diagnosis reagent, a biosensor, and a biomarker.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is the C₁-C₆₀ alkyl group), and non-limiting examples thereof include a methoxy group, an ethoxy group, and an iso-propyloxy (iso-propoxy) group.

The term “C₂-C₆₀ alkenyl group” as used herein refers to a hydrocarbon group formed by including at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by including at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and non-limiting examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom selected from N, O, P, Si and S as a ring-forming atom and 1 to 10 carbon atoms, and non-limiting examples thereof include a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and that has no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom selected from N, O, P, Si, and S as a ring-forming atom, 1 to 10 carbon atoms, and at least one carbon-carbon double bond in its ring. Examples of the C₁-C₁₀ heterocycloalkenyl group are a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has at least one heteroatom selected from N, O, P, Si, and S as a ring-forming atom, and 1 to 60 carbon atoms. The term “C₁-C₆₀ heteroarylene group” as used herein refers to a divalent group having a heterocyclic aromatic system that has at least one heteroatom selected from N, O, P, Si, and S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, an isoquinolinyl group. When the C₁-C₆₀ heteroaryl group and the C₁-C₆₀ heteroarylene group each include two or more rings, the rings may be fused to each other.

The term “C₆-C₆₀ aryloxy group” as used herein indicates —OA₁₀₂ (wherein A₁₀₂ is the C₆-C₆₀ aryl group), the term a C₆-C₆₀ arylthio group as used herein indicates —SA₁₀₃ (wherein A₁₀₃ is the C₆-C₆₀ aryl group), and the term “C₇-C₆₀ arylalkyl group” as used herein indicates -A₁₀₄A₁₀₅ (wherein A₁₀₄ is the C₆-C₅₉ aryl group and A₁₀₅ is the C₁-C₅₃ alkyl group).

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group include a fluorenyl group. The term “divalent non-aromatic condensed polycyclic group,” as used herein, refers to a divalent group having the same structure as the monovalent non-aromatic condensed polycyclic group.

The term “monovalent non-aromatic condensed heteropolycyclic group” as used herein refers to a monovalent group (for example, having 2 to 60 carbon atoms) having two or more rings condensed to each other, a heteroatom selected from N, O, P, Si, and S, other than carbon atoms, as a ring-forming atom, and no aromaticity in its entire molecular structure. Non-limiting examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₃₀ carbocyclic group” as used herein refers to a saturated or unsaturated cyclic group having, as a ring-forming atom, 5 to 30 carbon atoms only. The C₅-C₃₀ carbocyclic group may be a monocyclic group or a polycyclic group.

The term “C₁-C₃₀ heterocyclic group” as used herein refers to a saturated or unsaturated cyclic group having, as a ring-forming atom, at least one heteroatom selected from N, O, Si, P, and S other than 1 to 30 carbon atoms. The C₁-C₃₀ heterocyclic group may be a monocyclic group or a polycyclic group.

At least one substituent of the substituted C₅-C₃₀ carbocyclic group, the substituted C₂-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₂-C₆₀ heteroaryloxy group, the substituted C₂-C₆₀ heteroarylthio group, the substituted C₃-C₆₀ heteroarylalkyl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇) and —P(═O)(Q₃₈)(Q₃₉), and

Q₁ to Q₉, Q₁₁ to Q₁₉, Q₂₁ to Q₂₉, and Q₃₁ to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

When a group containing a specified number of carbon atoms is substituted with any of the groups listed in the preceding paragraph, the number of carbon atoms in the resulting “substituted” group is defined as the sum of the carbon atoms contained in the original (unsubstituted) group and the carbon atoms (if any) contained in the substituent.

For example, when the term “substituted C₁-C₃₀ alkyl” refers to a C₁-C₃₀ alkyl group substituted with C₆-C₃₀ aryl group, the total number of carbon atoms in the resulting aryl substituted alkyl group is C₇-C₆₀.

Hereinafter, a compound and an organic light-emitting device according to embodiments are described in detail with reference to Synthesis Examples and Examples. However, the organic light-emitting device is not limited thereto. The wording “B was used instead of A” used in describing Synthesis Examples refers to that an identical molar equivalent of B was used in place of A.

### EXAMPLE

### Synthesis Example 1

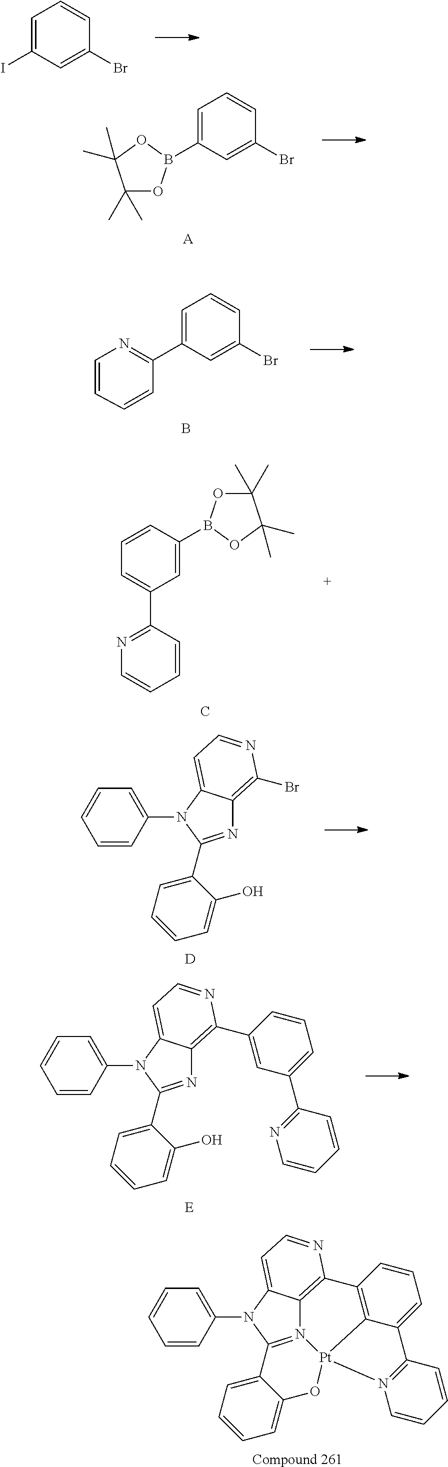

### Synthesis of Intermediate A (2-(3-bromophenyl)-4,4,5,5-tetramethyl-1,3,2-dioxaborolane)

10 grams (g) (0.035 moles, mol) of 1-bromo-3-iodobenzene and 13.5 g (0.053 mol, 1.5 equivalents, equiv.) of bis(pinacolato)diboron were added to a flask, and 6.9 g (0.071 mol, 2 equiv.) of potassium acetate and 1.44 g (0.05 equiv.) of PdCl₂(dppf) were added thereto. Then, 100 milliliters (mL) of toluene was added thereto, and the resultant mixture was refluxed at a temperature of 100° C. overnight. The refluxed mixture thus obtained was cooled to room temperature, and a precipitate was filtered therefrom. A filtrate obtained therefrom was washed by using EA/H₂O, and column chromatography was performed thereon to obtain 5.4 g (yield: 54%) of Intermediate A. The obtained product was confirmed by Mass and HPLC analysis.

HRMS (MALDI) calcd for C₁₂H₁₆BBrO₂: m/z 282.0427, Found: 282.0428.

### Synthesis of Intermediate B (2-(3-bromophenyl)pyridine)

5.4 g (0.019 mol, 1.2 equiv.) of the synthesized Intermediate A, 2.5 g (0.016 mol, 1 equiv.) of 2-bromopyridine, 1.28 g (0.001 mol, 0.07 equiv.) of tetrakis(triphenylphosphine)palladium(0), and 4.19 g (0.040 mol, 2.5 equiv.) of sodium carbonate were mixed with 60 mL of a solvent (0.6 molar, M) in which toluene, distilled water (H₂O), and ethanol (EtOH) were mixed at a ratio of 3:1:1, and then refluxed for 12 hours. The refluxed mixture thus obtained was cooled to room temperature, and a precipitate was filtered therefrom. A filtrate obtained therefrom was washed by using EA/H₂O, and column chromatography was performed thereon (while increasing a rate of MC/Hex to between 25% and 50%) to obtain 3 g (yield: 80%) of Intermediate B. The obtained product was confirmed by Mass and HPLC analysis.

HRMS (MALDI) calcd for C₁₁H₈BrN: m/z 232.9840, Found: 232.9842.

### Synthesis of Intermediate C (2-(3-(4,4,5,5-tetramethyl-1,3,2-dioxaborolan-2-yl)phenyl)pyridine)

3 g (0.013 mol) of Intermediate B (2-(3-bromophenyl)pyridine) and 4.9 g (0.019 mol, 1.5 equiv.) of bis(pinacolato)diboron were added to a flask, and 2.5 g (0.026 mol, 2 equiv.) of potassium acetate and 0.52 g (0.05 equiv.) of PdCl₂(dppf) were added thereto.

Then, 42 mL of toluene was added thereto, and the resultant mixture was refluxed at a temperature of 100° C. overnight. The refluxed mixture thus obtained was cooled to room temperature, and a precipitate was filtered therefrom. A filtrate obtained therefrom was washed by using EA/H₂O, and column chromatography was performed thereon to obtain 2.3 g (yield: 63%) of Intermediate C. The obtained product was confirmed by Mass and HPLC analysis.

HRMS (MALDI) calcd for C₁₇H₂₀BNO₂: m/z 281.1587, Found: 281.1587.

### Synthesis of Intermediate E (2-(1-phenyl-4-(3-(pyridin-2-yl)phenyl)-1H-imidazo[4,5-c]pyridin-2-yl)phenol)

2.3 g (0.008 mol, 1.2 equiv.) of the synthesized Intermediate C, 2.5 g (0.007 mol, 1 equiv.) of Intermediate D (2-(4-bromo-1-phenyl-1H-imidazo[4,5-c]pyridin-2-yl)phenol), 0.55 g (0.001 mol, 0.07 equiv.) of tetrakis(triphenylphosphine)palladium(0), and 2.8 g (0.020 mol, 3 equiv.) of potassium carbonate were mixed with 23 mL of a solvent in which tetrahydrofuran (THF) and distilled water (H₂O) were mixed at a ratio of 3:1, and then refluxed for 12 hours. The refluxed mixture thus obtained was cooled to room temperature, and a precipitate was filtered therefrom. A filtrate obtained therefrom was washed by using EA/H₂O, and column chromatography (while increasing a rate of MC/Hex to between 20% and 35%) was performed thereon to obtain 2.2 g (yield: 73%) of Intermediate E. The obtained product was confirmed by Mass and HPLC analysis.

HRMS (MALDI) calcd for C₂₉H₂₀N₄O: m/z 440.1637, Found: 440.1638.

### Synthesis of Compound 261

2.2 g (5 mmol) of Intermediate E and 2.5 g (6 mmol, 1.2 equiv.) of K₂PtCl₄ were mixed with 25 mL of a solvent in which 20 mL of AcOH and 5 mL of H₂O were mixed, and then refluxed for 16 hours. The refluxed mixture thus obtained therefrom was cooled to room temperature, and a precipitate was filtered therefrom. The precipitate was dissolved again in MC and washed by using H₂O. Then, column chromatography (MC 40%, EA 1%, Hex 59%) was performed thereon to obtain 1.2 g (purity: 99% or more) of Compound 261. The obtained product was confirmed by Mass and HPLC analysis.

HRMS (MALDI) calcd for C₂₉H₁₈N₄OPt: m/z 633.1128, Found: 633.1127.

### Synthesis Example 2

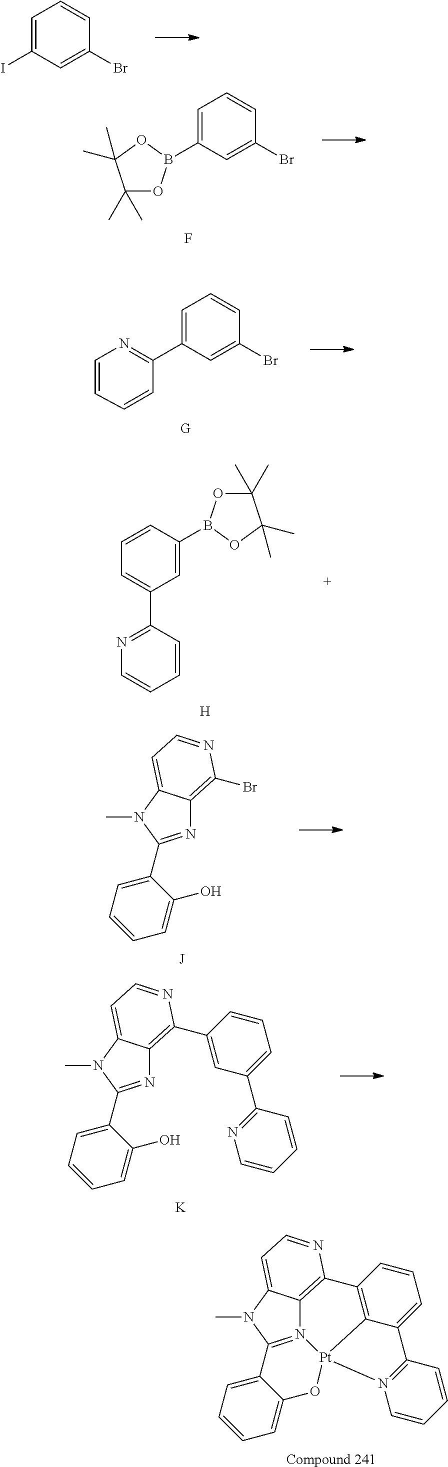

### Synthesis of Intermediate F (2-(3-bromophenyl)-4,4,5,5-tetramethyl-1,3,2-dioxaborolane)

10 g (0.035 mol) of 1-bromo-3-iodobenzene and 13.5 g (0.053 mol, 1.5 equiv.) of bis(pinacolato)diboron were added to a flask, and 6.9 g (0.071 mol, 2 equiv.) of potassium acetate and 1.44 g (0.05 equiv.) of PdCl₂(dppf) were added thereto. Then, 100 mL of toluene was added thereto, and the resultant mixture was refluxed at a temperature of 100° C. overnight. The refluxed mixture thus obtained was cooled to room temperature, and a precipitate was filtered therefrom. A filtrate obtained therefrom was washed by using EA/H₂O, and column chromatography was performed thereon to obtain 5.4 g (yield: 54%) of Intermediate F. The obtained product was confirmed by Mass and HPLC analysis.

HRMS (MALDI) calcd for C₁₂H₁₆BBrO₂: m/z 282.0427, Found: 282.0428.

### Synthesis of Intermediate G (2-(3-bromophenyl)pyridine)

5.4 g (0.019 mol, 1.2 equiv.) of the synthesized Intermediate F, 2.5 g (0.016 mol, 1 equiv.) of 2-bromopyridine, 1.28 g (0.001 mol, 0.07 equiv.) of tetrakis(triphenylphosphine)palladium(0), and 4.19 g (0.040 mol, 2.5 equiv.) of sodium carbonate were mixed with 60 mL of a solvent (0.6M) in which toluene, distilled water (H₂O), and ethanol (EtOH) were mixed at a ratio of 3:1:1, and then refluxed for 12 hours. The refluxed mixture thus obtained was cooled to room temperature, and a precipitate was filtered therefrom. A filtrate obtained therefrom was washed by using EA/H₂O, and column chromatography (while increasing a rate of MC/Hex to between 25% and 50%) was performed thereon to form 3 g (yield: 80%) of Intermediate G. The obtained product was confirmed by Mass and HPLC analysis.

HRMS (MALDI) calcd for C₁₁H₈BrN: m/z 232.9840, Found: 232.9842.

### Synthesis of Intermediate H (2-(3-(4,4,5,5-tetramethyl-1,3,2-dioxaborolan-2-yl)phenyl)pyridine)

3 g (0.013 mol) of Intermediate G (2-(3-bromophenyl)pyridine) and 4.9 g (0.019 mol, 1.5 equiv.) of bis(pinacolato)diboron were added to a flask, and 2.5 g (0.026 mol, 2 equiv.) of potassium acetate and 0.52 g (0.05 equiv.) of PdCl₂(dppf) were added thereto. Then, 42 mL of toluene was added thereto, and the resultant mixture was refluxed at a temperature of 100° C. overnight. The refluxed mixture thus obtained was cooled to room temperature, and a precipitate was filtered therefrom. A filtrate obtained therefrom was washed by using EA/H₂O, and column chromatography was performed thereon to obtain 2.3 g (yield: 63%) of Intermediate H. The obtained product was confirmed by Mass and HPLC analysis.

HRMS (MALDI) calcd for C₁₇H₂₀BNO₂: m/z 281.1587, Found: 281.1587.

### Synthesis of Intermediate K (2-(1-phenyl-4-(3-(pyridin-2-yl)phenyl)-1H-imidazo[4,5-c]pyridin-2-yl)phenol)

2.3 g (0.008 mol, 1.2 equiv.) of the synthesized Intermediate C, 2.0 g (0.007 mol, 1 equiv.) of Intermediate J (2-(4-bromo-1-methyl-1H-imidazo[4,5-c]pyridin-2-yl)phenol), 0.53 g (0.001 mol, 0.07 equiv.) of tetrakis(triphenylphosphine)palladium(0), and 2.7 g (0.020 mol, 3 equiv.) of potassium carbonate were mixed with 22 mL of a solvent in which THF and distilled water (H₂O) were mixed at a ratio of 3:1, and then refluxed for 12 hours. The refluxed mixture thus obtained was cooled to room temperature, and a precipitate was filtered therefrom. A filtrate obtained therefrom was washed by using EA/H₂O, and column chromatography was performed thereon (while increasing a rate of MC/Hex to between 20% and 35%) to obtain 1.7 g (yield: 70%) of Intermediate K. The obtained product was confirmed by Mass and HPLC analysis.

HRMS (MALDI) calcd for C₂₄H₁₈N₄O: m/z 378.1481, Found: 378.1482.

### Synthesis of Compound 241

1.7 g (3.8 mmol) of Intermediate K and 1.9 g (4.6 mmol, 1.2 equiv.) of K₂PtCl₄ were mixed with 25 mL of a solvent including 20 mL of AcOH and 5 mL of H₂O, and then refluxed for 16 hours. The refluxed mixture thus obtained was cooled to room temperature, and a precipitate was filtered therefrom. The precipitate was dissolved again in MC and washed by using H₂O. Then, column chromatography (MC 40%, EA 1%, Hex 59%) was performed thereon to obtain 1.0 g (purity: 99% or more) of Compound 241. The obtained product was confirmed by Mass and HPLC analysis.

HRMS (MALDI) calcd for C₂₄H₁₆N₄OPt: m/z 571.0972, Found: 571.0973.

Since the organometallic compound has excellent electrical characteristics and thermal stability, an organic light-emitting device including the organometallic compound has excellent driving voltage, luminescent efficiency, power efficiency, color purity, and lifespan characteristics. Also, since the organometallic compound has excellent phosphorescent emission characteristics, a diagnostic composition having high diagnostic efficiency may be provided by using the organometallic compound.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present description as defined by the following claims.

What is claimed is:

1. An organometallic compound represented by Formula 1: 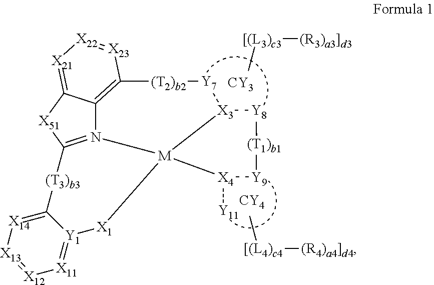
wherein, in Formula 1,
M is beryllium (Be), magnesium (Mg), aluminum (Al), calcium (Ca), titanium (Ti), manganese (Mn), cobalt (Co), copper (Cu), zinc (Zn), gallium (Ga), germanium (Ge), zirconium (Zr), ruthenium (Ru), rhodium (Rh), palladium (Pd), silver (Ag), rhenium (Re), platinum (Pt), or gold (Au),
X₁ is O or S, and a bond between X₁ and M is a covalent bond,
X₃ and X₄ are each independently N or C, a bond between N and M is a coordinate bond, and one bond selected from of a bond between X₃ and M and a bond between X₄ and M is a covalent bond and the other thereof is a coordinate bond,
Y₁, Y₇, Y₈, and Y₉ are each independently C or N,
Y₁₁ is C, N, O, or S,
a bond between X₃ and Y₇, a bond between X₃ and Y₈, a bond between X₄ and Y₉, and a bond between X₄ and Y₁₁ are each independently a single bond or a double bond,
CY₃ and CY₄ are each independently selected from a C₅-C₃₀ carbocyclic group and a C₁-C₃₀ heterocyclic group,
X₁₁ is N or C-[(L₁₁)_{c11}-(R₁₁)ₐ₁₁], X₁₂ is N or C-[(L₁₂)_{c12}-(R₁₂)ₐ₁₂], X₁₃ is N or C-[(L₁₃)_{c13}-(R₁₃)ₐ₁₃], X₁₄ is N or C-[(L₁₄)_{c14}-(R₁₄)ₐ₁₄], X₂₁ is N or C-[(L₂₁)_{c21}-(R₂₁)ₐ₂₁], X₂₂ is N or C-[(L₂₂)_{c22}-(R₂₂)ₐ₂₂], and X₂₃ is N or C-[(L₂₃)_{c23}-(R₂₃)ₐ₂₃], provided that at least one of X₁₁ to X₁₄ and X₂₁ to X₂₃ is N,
T₁ to T₃ are each independently selected from *—O—*′, *—S—*′, *—C(R₅)(R₆)—*, *—C(R₅)=*′, *═C(R₅)—*′, *—C(R₅)═C(R₆)—*′, *—C(═O)—*′, *—C(═S)—*′, *—C≡C—*′, *—N(R₅)—*′, *—Si(R₅)(R₆)—*′, and *—P(R₅)(R₆)—*,
R₅ and R₆ are optionally linked via a first linking group to form a substituted or unsubstituted C₅-C₃₀ carbocyclic group or a substituted or unsubstituted C₁-C₃₀ heterocyclic group,
b1 to b3 are each independently 0, 1, 2, or 3, wherein, when b1 is zero, *-(T₁)_{b1}-*′ is a single bond, when b1 is two or more, two or more groups T₁ are identical to or different from each other, when b2 is zero, *-(T₂)_{b2}-*′ is a single bond, when b2 is two or more, two or more groups T₂ are identical to or different from each other, when b3 is zero, *-(T₃)_{b3}-*′ is a single bond, and when b3 is two or more, two or more groups T₃ are identical to or different from each other,
X₅₁ is selected from O, S, N-[(L₇)_{c7}-(R₇)ₐ₇], C(R₇)(R₈), Si(R₇)(R₈), and C(═O),
R₇ and R₈ are optionally linked via a second linking group to form a substituted or unsubstituted C₅-C₃₀ carbocyclic group or a substituted or unsubstituted C₁-C₃₀ heterocyclic group,
L₃, L₄, L₇, L₁₁ to L₁₄, and L₂₁ to L₂₃ are each independently selected from a single bond, a substituted or unsubstituted C₅-C₃₀ carbocyclic group, and a substituted or unsubstituted C₁-C₃₀ heterocyclic group,
c3, c4, c7, cl11 to c14, and c21 to c23 are each independently an integer from 1 to 5,
R₃ to R₈, R₁₁ to R₁₄, and R₂₁ to R₂₃ are each independently selected from hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₂-C₆₀ heteroaryloxy group, a substituted or unsubstituted C₂-C₆₀ heteroarylthio group, a substituted or unsubstituted C₃-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),
a3, a4, a7, a11 to a14, a21 to a23, d3, and d4 are each independently 0, 1, 2, 3, 4, or 5,
two of *-(L₁₁)_{c11}-(R₁₁)ₐ₁₁, *-(L₁₂)_{c12}-(R₁₂)ₐ₁₂, *-(L₁₃)_{c13}-(R₁₃)ₐ₁₃, and *-(L₁₄)_{c14}-(R₁₄)ₐ₁₄ are optionally linked to form a substituted or unsubstituted C₅-C₃₀ carbocyclic group or a substituted or unsubstituted C₁-C₃₀ heterocyclic group,
two of *-(L₂₁)_{c21}-(R₂₁)ₐ₂₁, *-(L₂₂)_{c22}-(R₂₂)ₐ₂₂, and *-(L₂₃)_{c23}-(R₂₃)ₐ₂₃ are optionally linked to form a substituted or unsubstituted C₅-C₃₀ carbocyclic group or a substituted or unsubstituted C₁-C₃₀ heterocyclic group,
two of groups *-(L₃)_{c3}-(R₃)ₐ₃ in the number of d3 are optionally linked to form a substituted or unsubstituted C₅-C₃₀ carbocyclic group or a substituted or unsubstituted C₁-C₃₀ heterocyclic group,
two of groups *-(L₄)_{c4}-(R₄)ₐ₄ in the number of d4 are optionally linked to form a substituted or unsubstituted C₅-C₃₀ carbocyclic group or a substituted or unsubstituted C₁-C₃₀ heterocyclic group,
* and *′ each indicate a binding site to a neighboring atom,
at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₂-C₆₀ heteroaryloxy group, the substituted C₂-C₆₀ heteroarylthio group, the substituted C₃-C₆₀ heteroarylalkyl group, the substituted monovalent non-aromatic condensed polycyclic group, and the substituted monovalent non-aromatic condensed heteropolycyclic group is selected from:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and
—N(Q₃₁)(Q₃₂), —Si(Q₃₃)(Q₃₄)(Q₃₅), —B(Q₃₆)(Q₃₇), and —P(═O)(Q₃₈)(Q₃₉), and
Q₁ to Q₉, Q₁₁ to Q₁₉, Q₂₁ to Q₂₉, and Q₃₁ to Q₃₉ are each independently selected from hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₂-C₆₀ heteroaryloxy group, a C₂-C₆₀ heteroarylthio group, a C₃-C₆₀ heteroarylalkyl group, a monovalent non-aromatic condensed polycyclic group, and a monovalent non-aromatic condensed heteropolycyclic group.

2. The organometallic compound of claim 1, wherein
X₃ is C, X₄ is N, a bond between X₃ and M is a covalent bond, and a bond between X₄ and M is a coordinate bond, and
Y₁, Y₇, Y₈, and Y₉ are each C.

3. The organometallic compound of claim 1, wherein
CY₃ is selected from a benzene group, a naphthalene group, an anthracene group, a phenanthrene group, a triphenylene group, a pyrene group, a chrysene group, a cyclopentadiene group, an indene group, a fluorene group, a pyrrole group, an indole group, a carbazole group, a furan group, a benzofuran group, a dibenzofuran group, a thiophene group, a benzothiophene group, a dibenzothiophene group, a pyridine group, a pyrimidine group, and a 1,2,3,4-tetrahydronaphthalene group, and
CY₄ is selected from a pyridine group, a pyrimidine group, a pyrazine group, a pyridazine group, a triazine group, a quinoline group, an isoquinoline group, a quinoxaline group, a quinazoline group, a phenanthroline group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, and a 5,6,7,8-tetrahydroquinoline group.

4. The organometallic compound of claim 1, wherein
T₁ to T₃ are each independently selected from *—O—*′, *—S—*′, *—C(R₅)(R₆)—*, *—N(R₅)—*′, *—Si(R₅)(R₆)—*′, and *—P(R₅)(R₆)—*.

5. The organometallic compound of claim 1, wherein
a11 of b1, b2, and b3 are 0;
b1 is 1 and b2 and b3 are each 0;
b2 is 1 and b1 and b3 are each 0; or
b3 is 1 and b1 and b2 are each 0.

6. The organometallic compound of claim 1, wherein
L₃, L₄, L₇, L₁₁ to L₁₄, and L₂₁ to L₂₃ are each independently selected from:
a single bond, a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, and a benzothiadiazole group; and
a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, and a benzothiadiazole group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a pyridinyl group, a pyrimidinyl group, a triazinyl group, a fluorenyl group, a dimethylfluorenyl group, a diphenylfluorenyl group, a carbazolyl group, a phenylcarbazolyl group, a dibenzofuranyl group, a dibenzothiophenyl group, a dibenzosilolyl group, a dimethyldibenzosilolyl group, a diphenyldibenzosilolyl group, —N(Q₃₁)(Q₃₂), —Si(Q₃₃)(Q₃₄)(Q₃₅), —B(Q₃₆)(Q₃₇), and —P(═O)(Q₃₈)(Q₃₉), and
Q₃₁ to Q₃₉ are each independently selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group; and
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group, each substituted with at least one selected from deuterium, a C₁-C₁₀ alkyl group, and a phenyl group.

7. The organometallic compound of claim 1, wherein
R₃ to R₈, R₁₁ to R₁₄, and R₂₁ to R₂₃ are each independently selected from:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, and a C₁-C₂₀ alkoxy group;
a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and
—N(Q₁)(Q₂), —Si(Q₃)(Q₄)(Q₅), —B(Q₆)(Q₇), and —P(═O)(Q₈)(Q₉), and
Q₁ to Q₉ are each independently selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

8. The organometallic compound of claim 1, wherein
R₃ to R₈, R₁₁ to R₁₄, and R₂₁ to R₂₃ are each independently selected from:
hydrogen, deuterium, —F, a cyano group, a nitro group, —SF₅, a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group;
a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group, each substituted with at least one selected from deuterium, —F, —CD₃, —CD₂H, —CDH₂, —CF₃, —CF₂H, —CFH₂, a cyano group, a nitro group, a C₁-C₁₀ alkyl group, a C₁-C₁₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group; and
—N(Q₁)(Q₂), —Si(Q₃)(Q₄)(Q₅), —B(Q₆)(Q₇), and —P(═O)(Q₈)(Q₉), and
Q₁ to Q₉ are each independently selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an isopropyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

9. The organometallic compound of claim 1, wherein
R₃ to R₈, R₁₁ to R₁₄, and R₂₁ to R₂₃ are each independently selected from hydrogen, deuterium, —F, a cyano group, a nitro group, —SF₅, —CH₃, —CD₃, —CD₂H, —CDH₂, —CF₃, —CF₂H, —CFH₂, groups represented by Formulae 9-1 to 9-19, groups represented by Formulae 10-1 to 10-139, and —Si(Q₃)(Q₄)(Q₅): 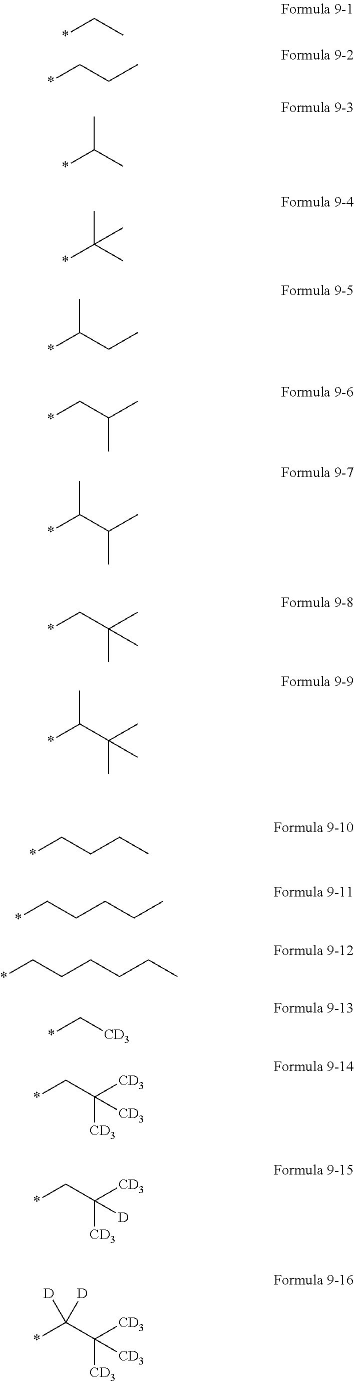 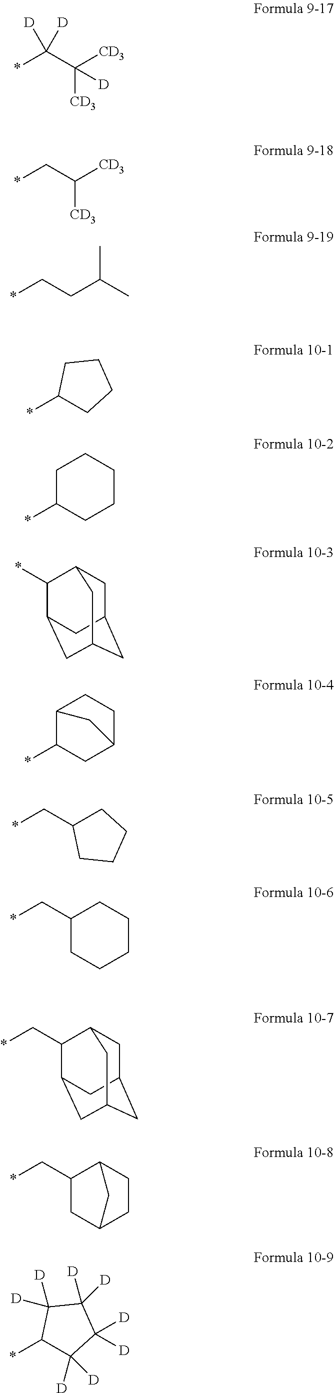 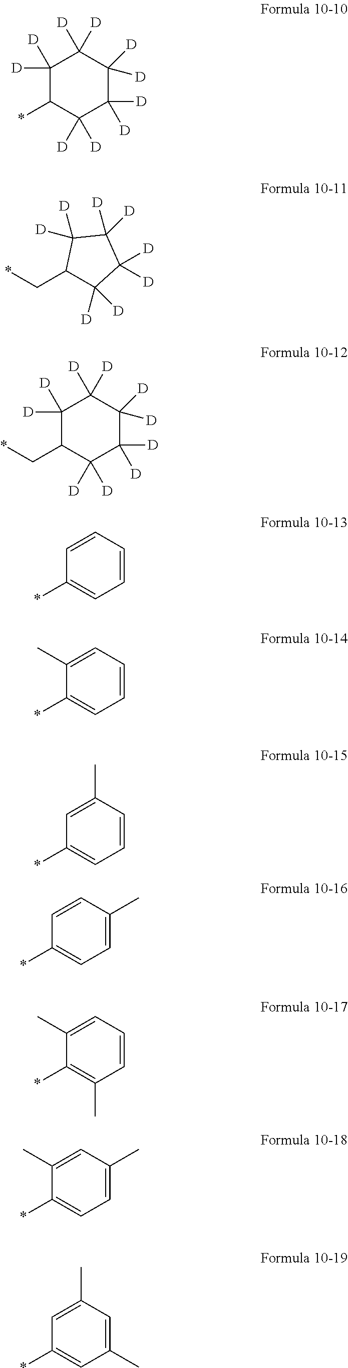 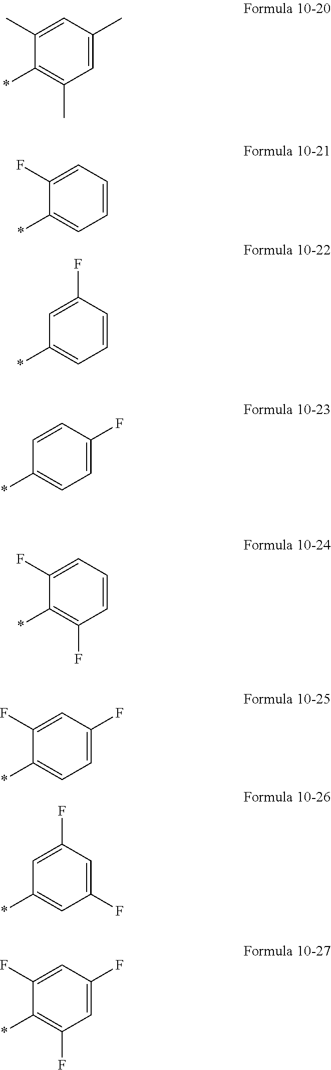 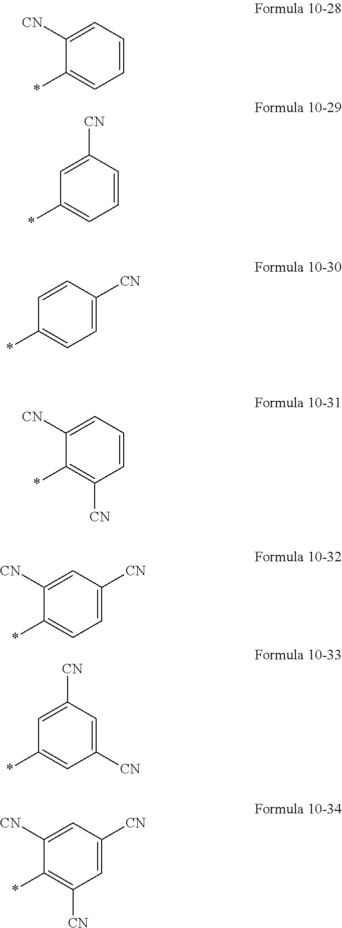 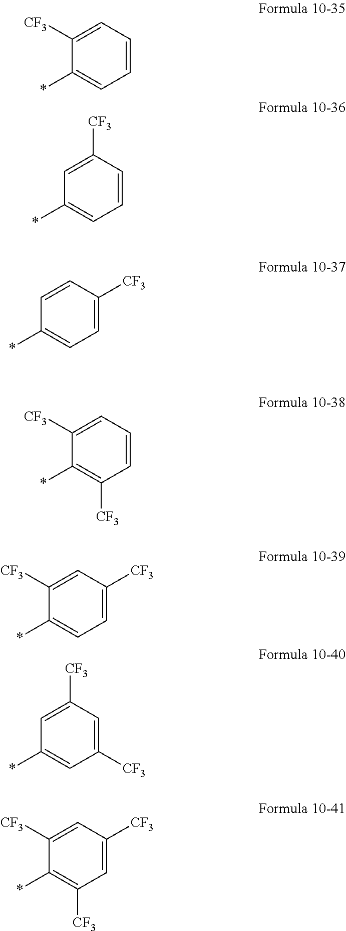 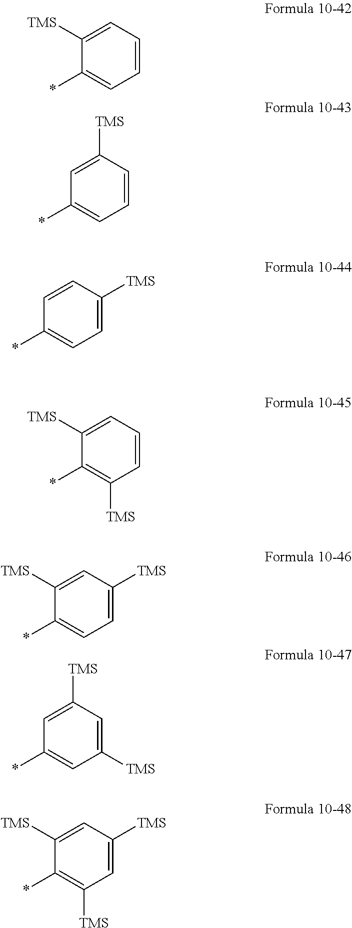 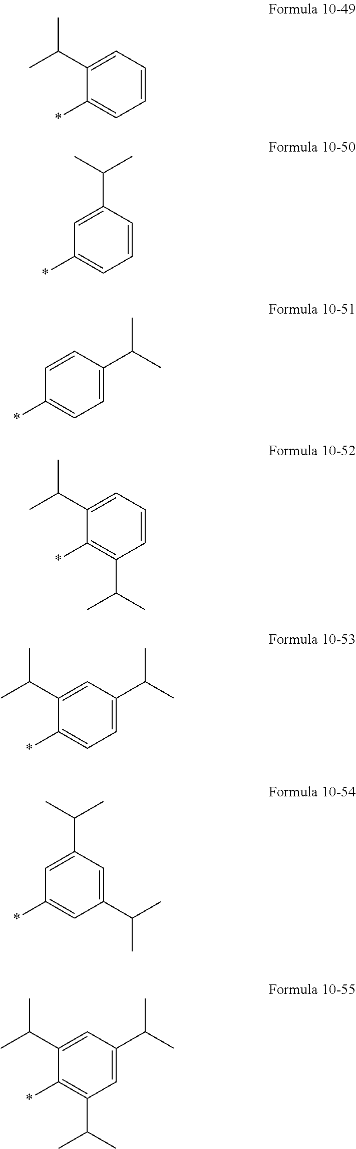 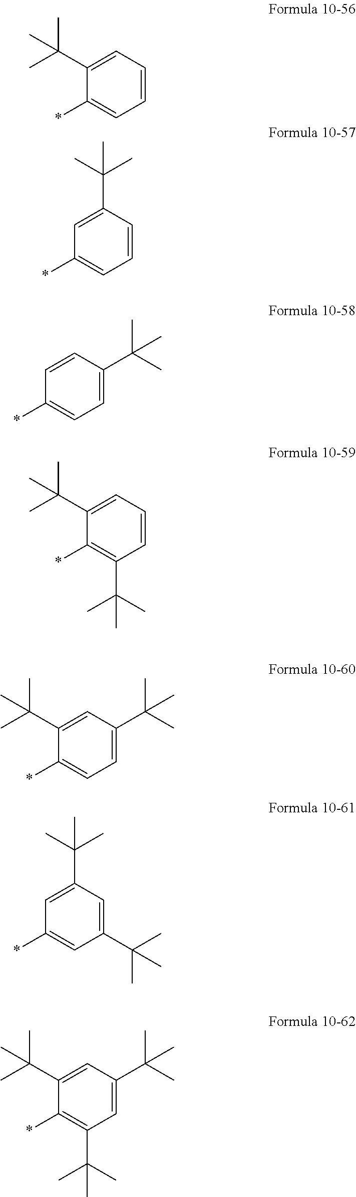 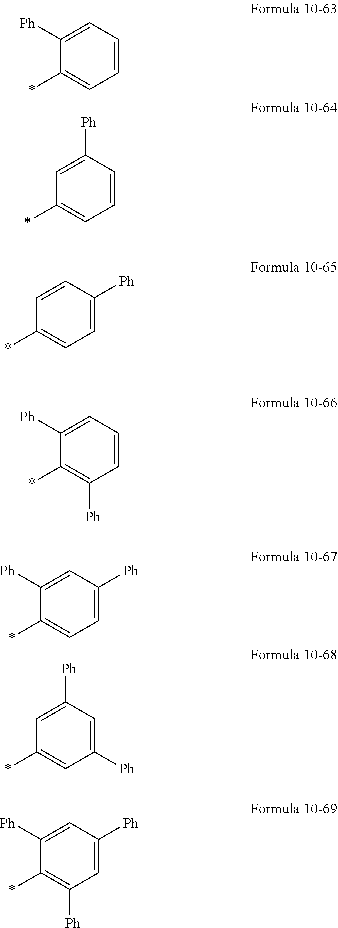 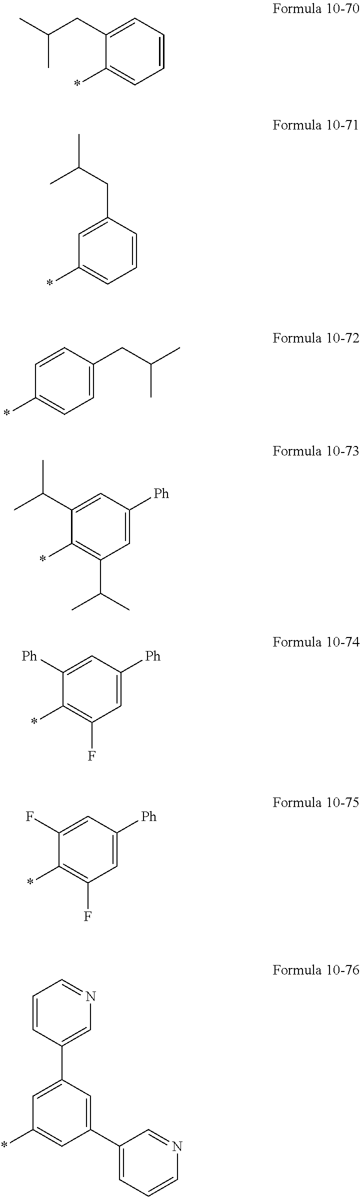 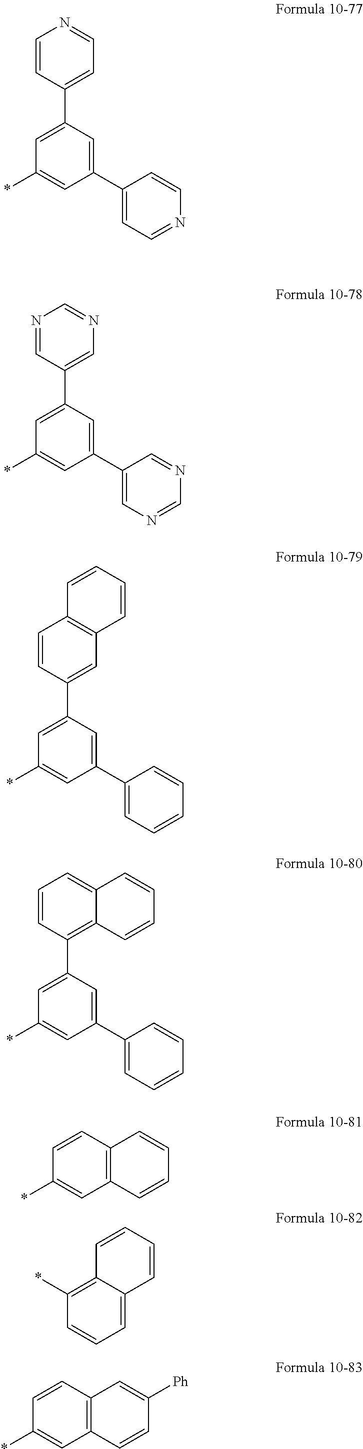 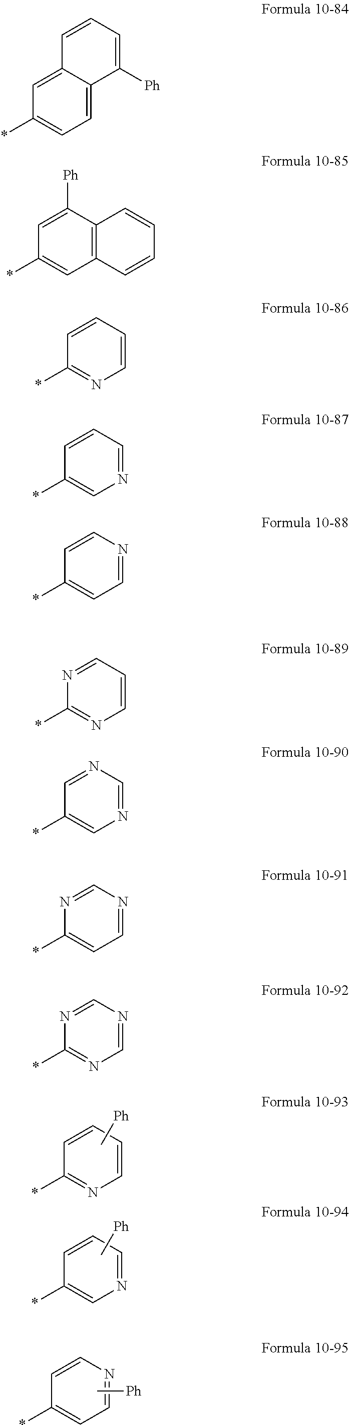 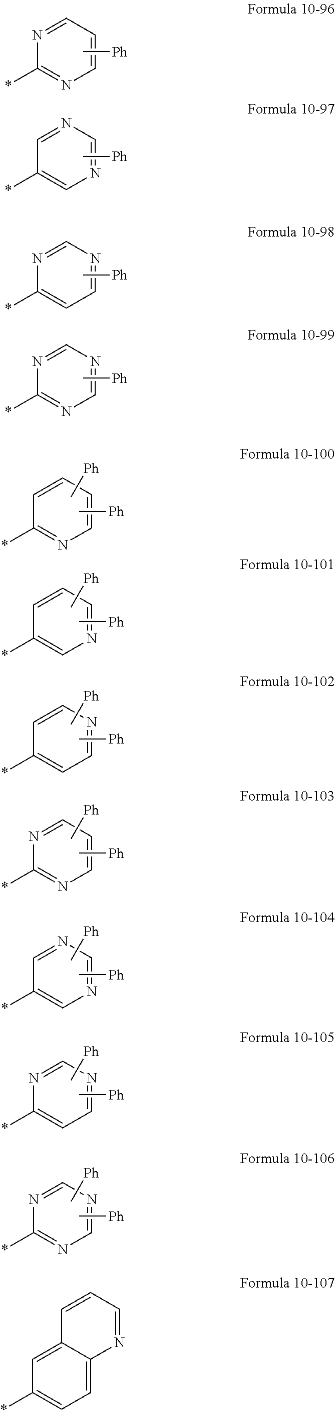 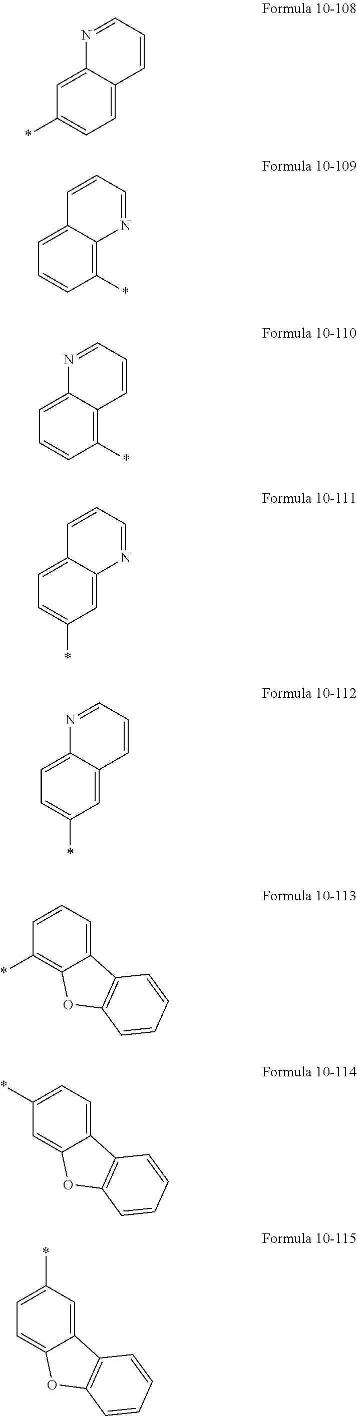 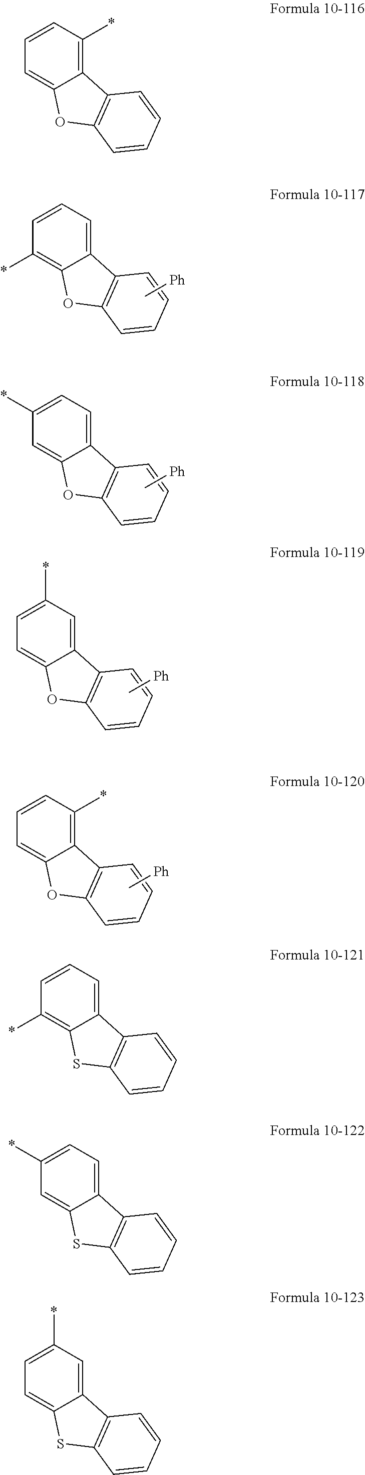 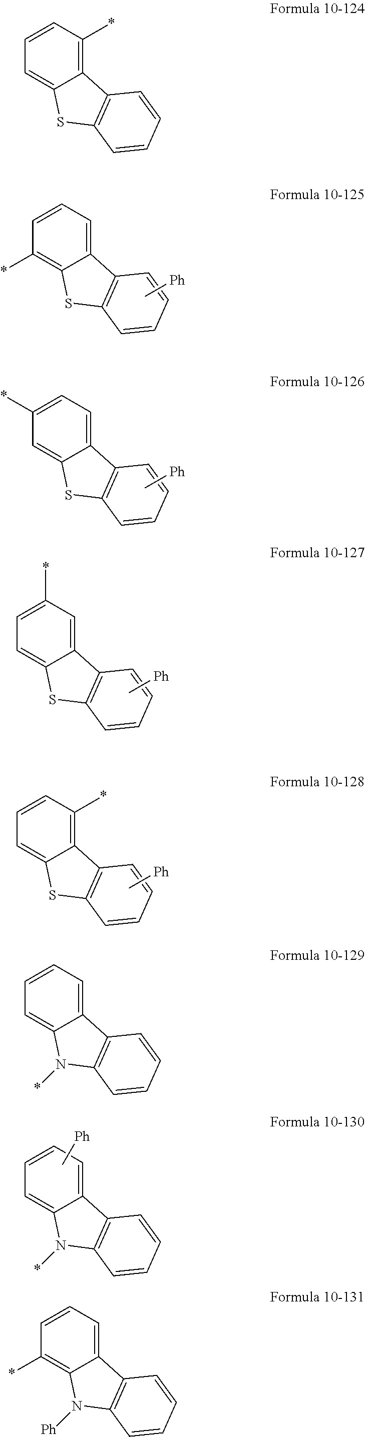 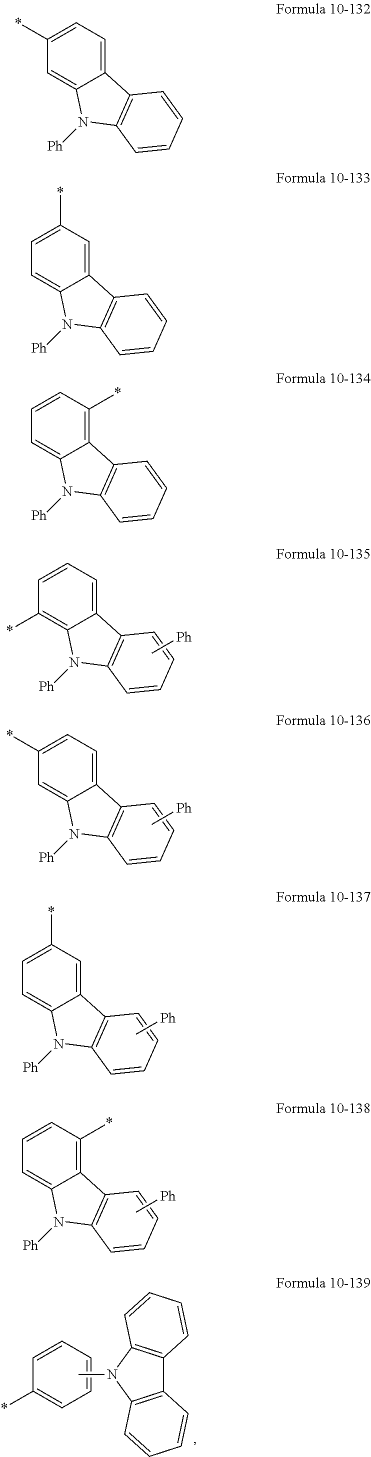
wherein Q₃ to Q₉ are each independently selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group; and
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, a biphenyl group, and a naphthyl group, each substituted with at least one selected from deuterium, a C₁-C₁₀ alkyl group, and a phenyl group,
wherein, in Formulae 9-1 to 9-19 and 10-1 to 10-139,
* indicates a binding site to a neighboring atom,
Ph indicates a phenyl group, and
TMS indicates a trimethylsilyl group.

10. The organometallic compound of claim 1, wherein
a moiety represented by 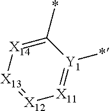
in Formula 1 is selected from groups represented by Formulae CY1-1 to CY1-16, and
a moiety represented by 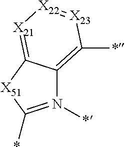
in Formula 1 is selected from Formulae CY2-1 to CY2-5: 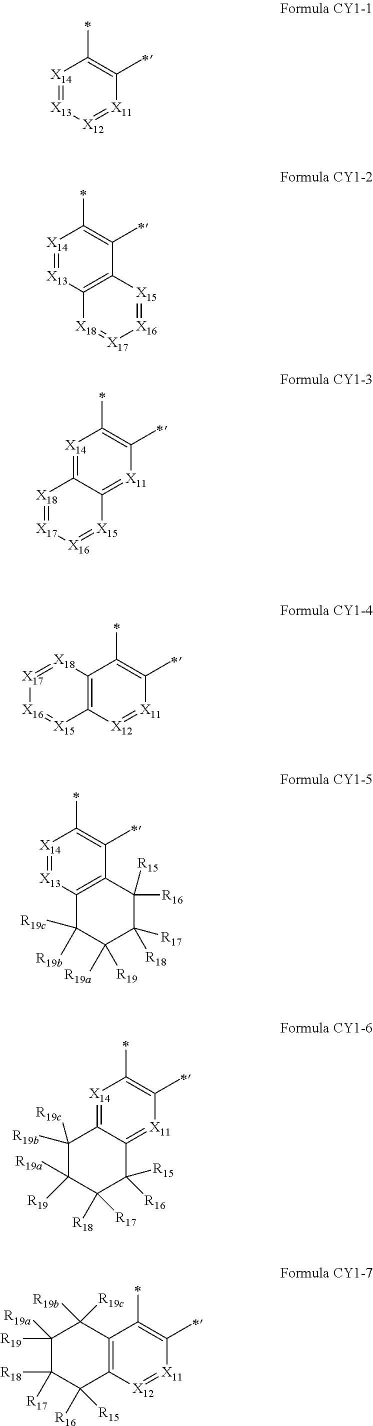 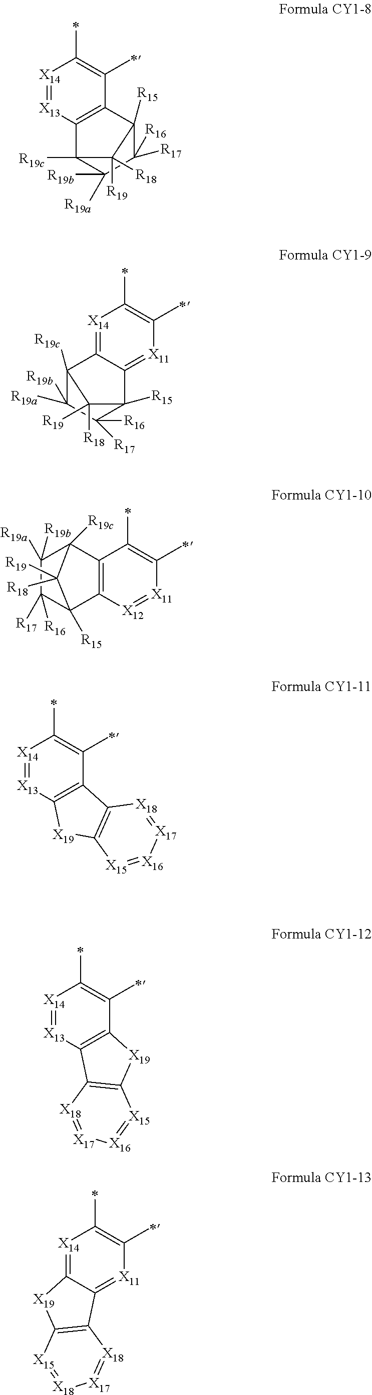 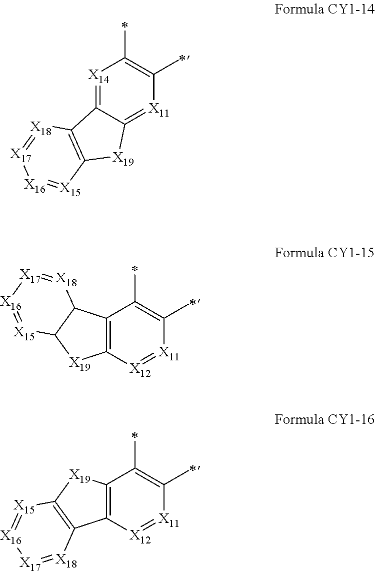
wherein, in Formulae CY1-1 to CY1-16,
X₁₁ is N or C-[(L₁₁)_{c11}-(R₁₁)ₐ₁₁], X₁₂ is N or C-[(L₁₂)_{c12}-(R₁₂)ₐ₁₂], X₁₃ is N or C-[(L₁₃)_{c13}-(R₁₃)ₐ₁₃], X₁₄ is N or C-[(L₁₄)_{c14}-(R₁₄)ₐ₁₄], X₁₅ is N or C-[(L₁₅)_{c15}-(R₁₅)ₐ₁₅], X₁₆ is N or C-[(L₁₆)_{c16}-(R₁₆)ₐ₁₆], X₁₇ is N or C-[(L₁₇)_{c17}-(R₁₇)ₐ₁₇], and X₁₈ is N or C-[(L₁₈)_{c18}-(R₁₈)ₐ₁₈],
X₁₉ is C(R₁₉ₐ)(R_{19b}), N-[(L₁₉)_{c19}-(R₁₉)ₐ₁₉], O, S, or Si(R₁₉ₐ)(R_{19b}),
L₁₁ to L₁₉ are each independently the same as described in connection with L₁₁ in claim 1,
c11 to c19 are each independently the same as described in connection with c11 in claim 1,
R₁₁ to R₁₉ and R₁₉ₐ to R_{19c} are each independently the same as described in connection with R₁₁ in claim 1,
a11 to a19 are each independently the same as described in connection with a11 in claim 1, and
* and *′ each indicate a binding site to a neighboring atom: 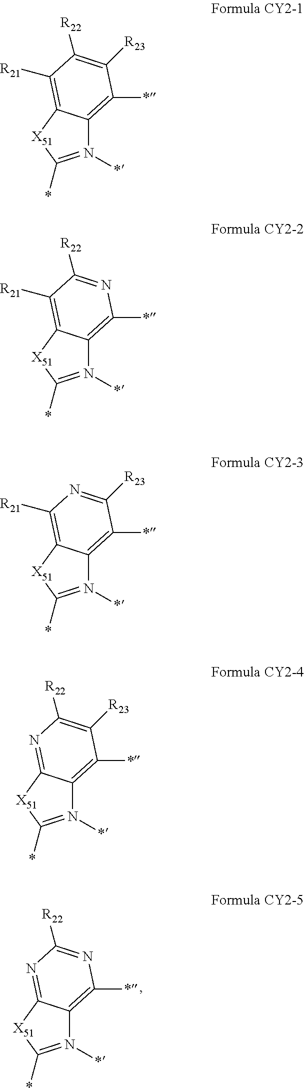
wherein, in Formulae CY2-1 to CY2-5,
X₅₁ is the same as described in connection with X₅₁ in claim 1,
R₂₁ to R₂₃ are each independently the same as described in connection with R₂₁ in claim 1, and
*, *′, and *″ each indicate a binding site to a neighboring atom.

11. The organometallic compound of claim 10, wherein
1) in Formula 1, a moiety represented by 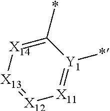
is selected from groups represented by Formulae CY1-1 to CY1-16, provided that i) at least one of X₁₁ to X₁₄ in Formula CY1-1 is N, at least one of X₁₃ and X₁₄ in Formulae CY1-2, CY1-5, CY1-8, CY1-11, and CY1-12 is N, at least one of X₁₁ and X₁₄ in Formulae CY1-3, CY1-6, CY1-9, CY1-13, and CY1-14 is N, and at least one of X₁₁ and X₁₂ in Formulae CY1-4, CY1-7, CY1-10, CY1-15, and CY1-16 is N, and ii) a moiety represented by 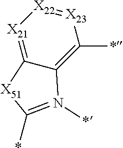
is a group represented by Formula CY2-1;
2) in Formula 1, a moiety represented by 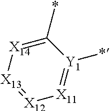
is selected from groups represented by Formulae CY1-1 to CY1-16, provided that i) all of X₁₁ to X₁₄ in Formulae CY1-1 to CY1-16 are not N, and ii) a moiety represented by 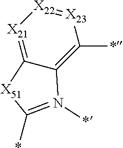
is selected from groups represented by Formulae CY2-2 to CY2-5; or
3) in Formula 1, a moiety represented by 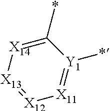
is selected from groups represented by Formulae CY1-1 to CY1-16, provided that i) at least one of X₁₁ to X₁₄ in Formula CY1-1 is N, at least one of X₁₃ and X₁₄ in Formulae CY1-2, CY1-5, CY1-8, CY1-11, and CY1-12 is N, at least one of X₁₁ and X₁₄ in Formulae CY1-3, CY1-6, CY1-9, CY1-13, and CY1-14 is N, and at least one of X₁₁ and X₁₂ in Formula CY1-4, CY1-7, CY1-10, CY1-15 and CY1-16 is N, and ii) a moiety represented by 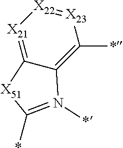
is selected from groups represented by Formulae CY2-2 to CY2-5.

12. The organometallic compound of claim 1, wherein
a moiety represented by 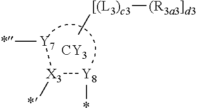
in Formula 1 is selected from groups represented by Formulae CY3-1 to CY3-22: 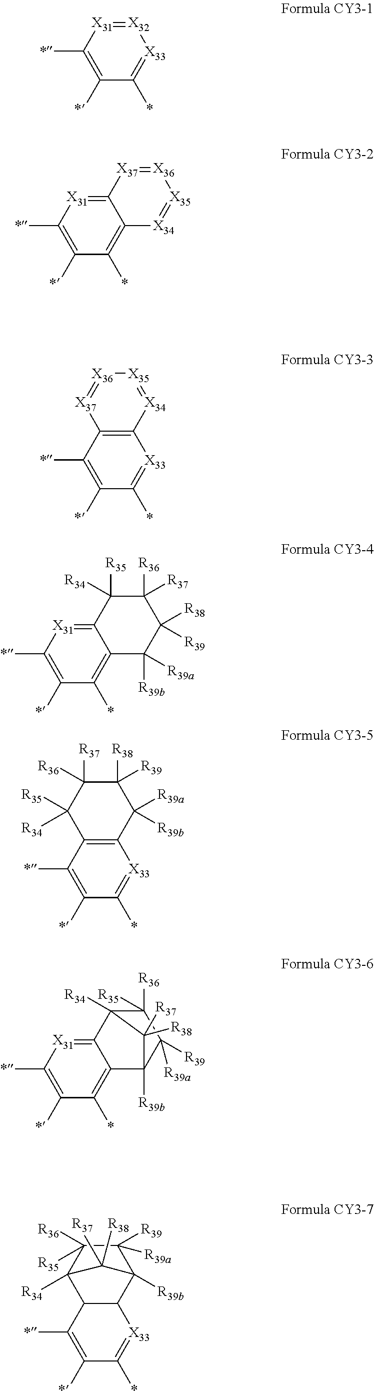 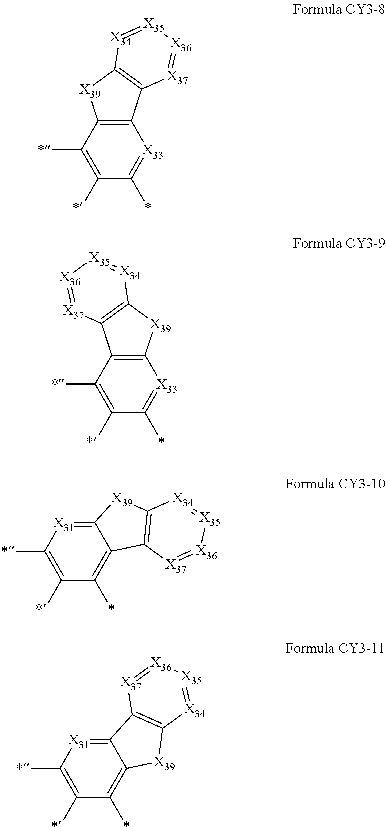 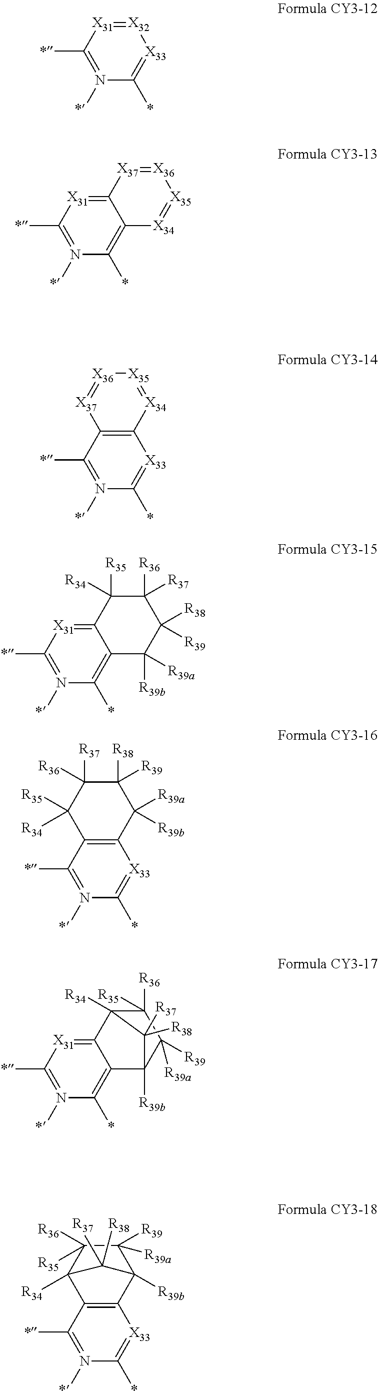 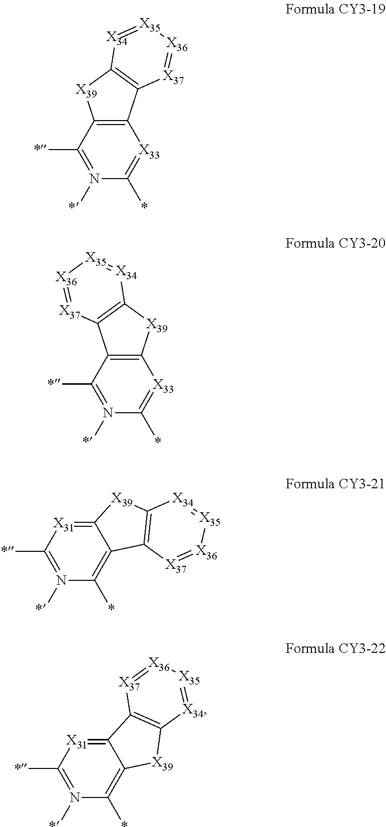
wherein, in Formulae CY3-1 to CY3-22,
X₃₁ is N or C-[(L₃₁)_{c31}-(R₃₁)ₐ₃₁], X₃₂ is N or C-[(L₃₂)_{c32}-(R₃₂)ₐ₃₂], X₃₃ is N or C-[(L₃₃)_{c33}-(R₃₃)ₐ₃₃], X₃₄ is N or C-[(L₃₄)_{c34}-(R₃₄)ₐ₃₄], X₃₅ is N or C-[(L₃₅)_{c35}-(R₃₅)ₐ₃₅], X₃₆ is N or C-[(L₃₆)_{c36}-(R₃₆)ₐ₃₆], and X₃₇ is N or C-[(L₃₇)_{c37}-(R₃₇)ₐ₃₇],
X₃₉ is C(R₃₉ₐ)(R_{39b}), N-[(L₃₉)_{c39}-(R₃₉)ₐ₃₉], O, S, or Si(R₃₉ₐ)(R_{39b}),
L₃₁ to L₃₉ are each independently the same as described in connection with L₃ in claim 1,
c31 to c39 are each independently the same as described in connection with c3 in claim 1,
R₃₁ to R₃₉ and R₃₉ₐ to R_{39b} are each independently the same as described in connection with R₃ in claim 1,
a31 to a39 are each independently the same as described in connection with a3 in claim 1, and
*, *′, and *″ each indicate a binding site to a neighboring atom.

13. The organometallic compound of claim 1, wherein
a moiety represented by 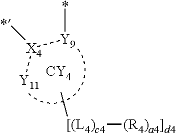
in Formula 1 is selected from groups represented by Formulae CY4-1 to CY4-8: 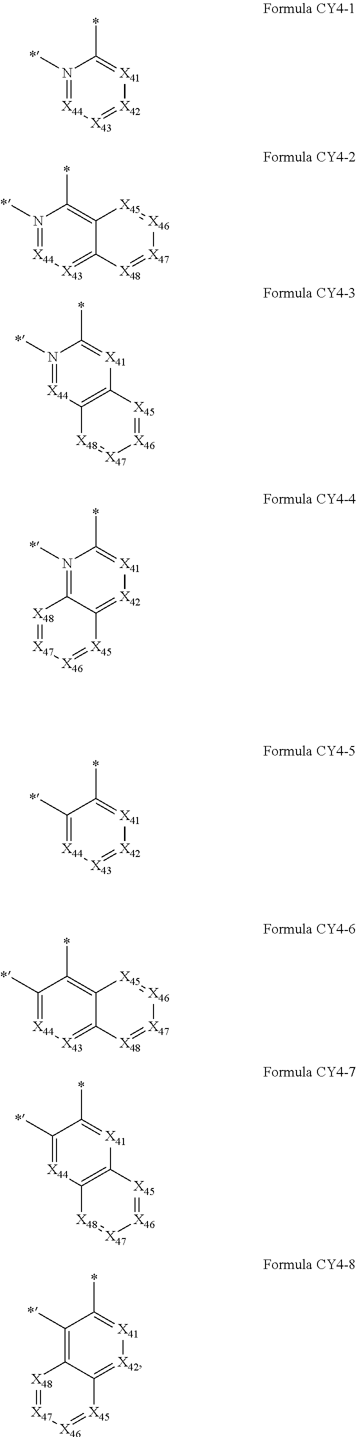
wherein, in Formulae CY4-1 to CY4-8,
X₄₁ is N or C-[(L₄₁)_{c41}-(R₄₁)ₐ₄₁], X₄₂ is N or C-[(L₄₂)_{c42}-(R₄₂)ₐ₄₂], X₄₃ is N or C-[(L₄₃)_{c43}-(R₄₃)ₐ₄₃], X₄₄ is N or C-[(L₄₄)_{c44}-(R₄₄)ₐ₄₄], X₄₅ is N or C-[(L₄₅)_{c45}-(R₄₅)ₐ₄₅], X₄₆ is N or C-[(L₄₆)_{c46}-(R₄₆)ₐ₄₆], X₄₇ is N or C-[(L₄₇)_{c47}-(R₄₇)ₐ₄₇], and X₄₈ is N or C-[(L₄₈)_{c48}-(R₄₈)ₐ₄₈],
L₄₁ to L₄₈ are each independently the same as described in connection with L₄ in claim 1,
c41 to c48 are each independently the same as described in connection with c4 in claim 1,
R₄₁ to R₄₈ are each independently the same as described in connection with R₄ in claim 1,
a41 to a48 are each independently the same as described in connection with a4 in claim 1, and
* and *′ each indicate a binding site to a neighboring atom.

14. The organometallic compound of claim 1, wherein
the organometallic compound is represented by Formula 1-1 or 1-2,
1) in Formulae 1-1 and 1-2, a moiety represented by 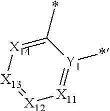
is selected from groups represented by Formulae CY1-1(2) to CY1-1(8), and a moiety represented by 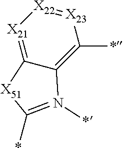
is a group represented by Formula CY2-1;
2) in Formulae 1-1 and 1-2, a moiety represented by 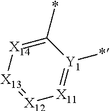
is a group represented by Formula CY1-1(1), and a moiety represented by 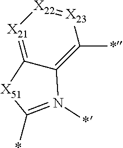
is selected from groups represented by Formulae CY2-2 to CY2-5; or
3) in Formulae 1-1 and 1-2, a moiety represented by 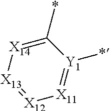
is selected from groups represented by Formulae CY1-1(2) to CY1-1(8), and a moiety represented by 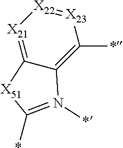
is selected from groups represented by Formulae CY2-2 to CY2-5: 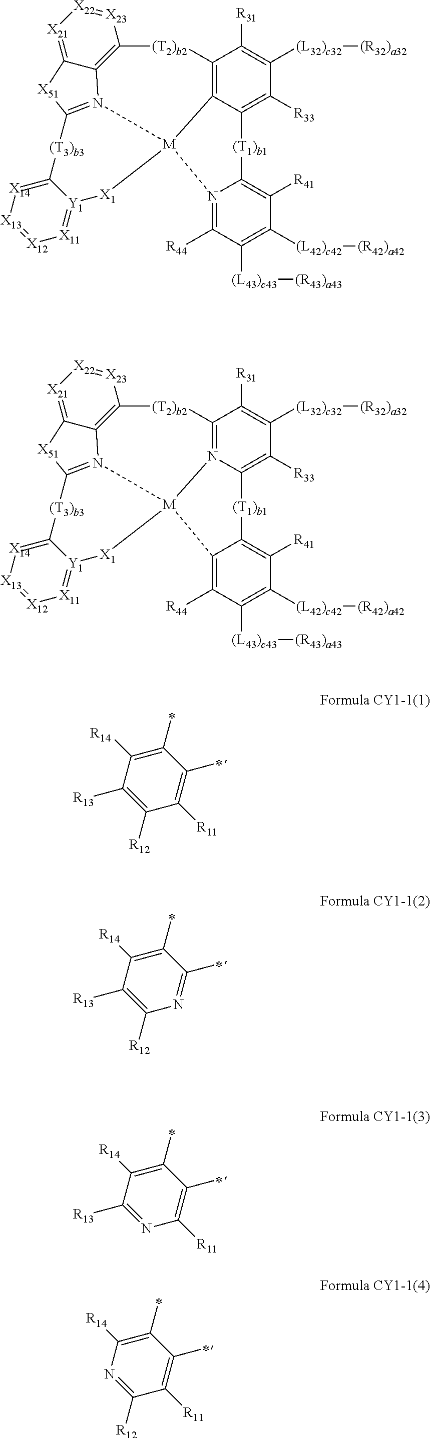 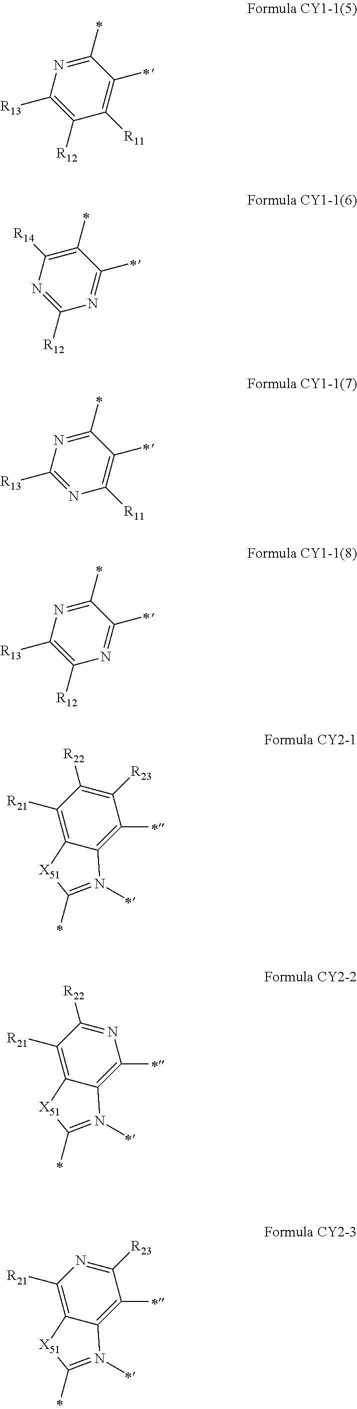 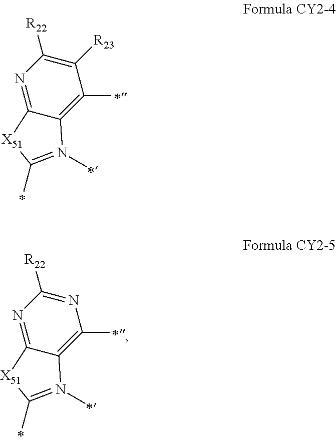
wherein, in Formulae 1-1, 1-2, CY1-1(1) to CY1-1(8), and CY2-1 to CY2-5,
M, X₁, Y₁, T₁ to T₃, b1 to b3, X₅₁, X₁₁ to X₁₄, and X₂₁ to X₂₃ are each independently the same as described in claim 1,
L₃₂ is the same as described in connection with L₃ in claim 1,
c32 is the same as described in connection with c3 in claim 1,
R₃₁ to R₃₃ are each independently the same as described in connection with R₃ in claim 1,
a32 is the same as described in connection with a3 in claim 1,
L₄₂ and L₄₃ are each independently the same as described in connection with L₄ in claim 1,
c42 and c43 are each independently the same as described in connection with c4 in claim 1,
R₄₁ to R₄₄ are each independently the same as described in connection with R₄ in claim 1,
a42 and a43 are each independently the same as described in connection with a4 in claim 1,
R₁₁ to R₁₄ are each independently the same as described in connection with R₁₁ in claim 1,
R₂₁ to R₂₃ are each independently the same as described in connection with R₂₁ in claim 1, and
*, *′, and *″ each indicate a binding site to a neighboring atom.

15. The organometallic compound of claim 1, wherein
the organometallic compound is one of Compounds 1 to 822:

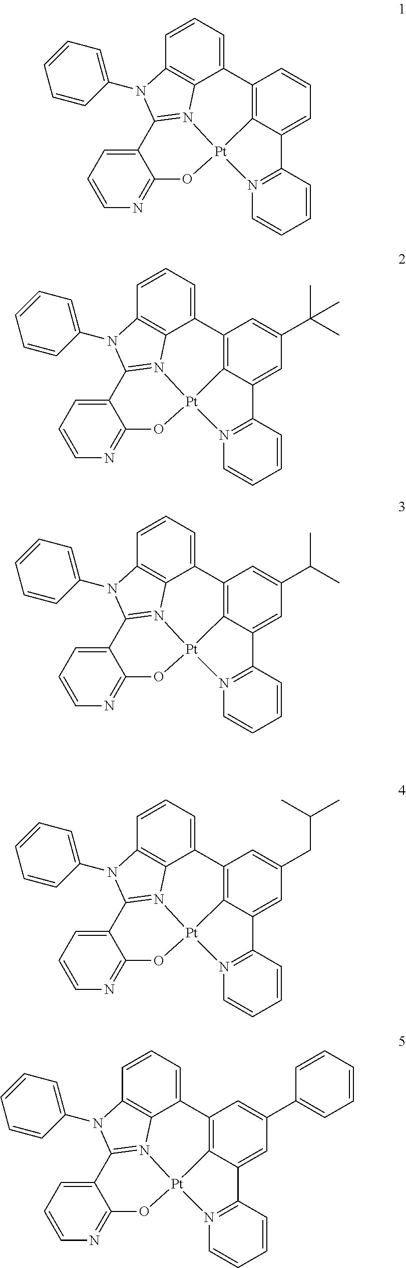

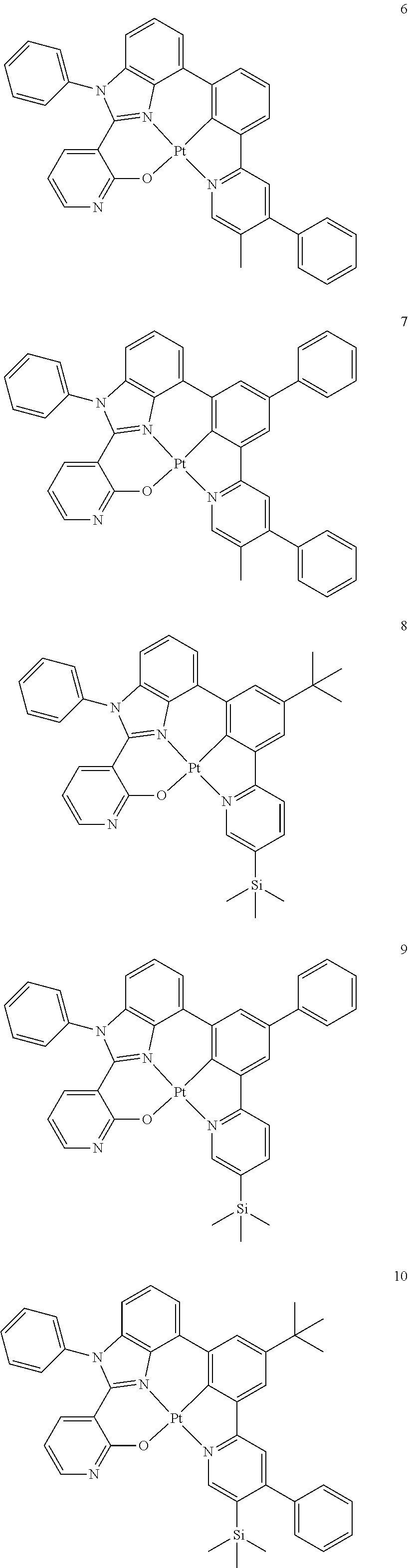

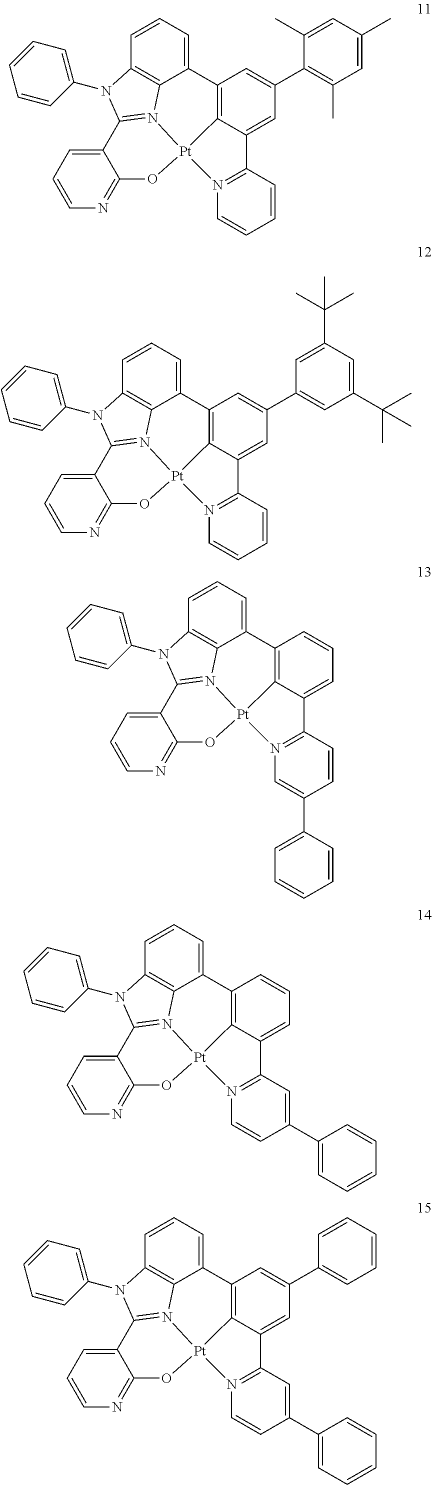

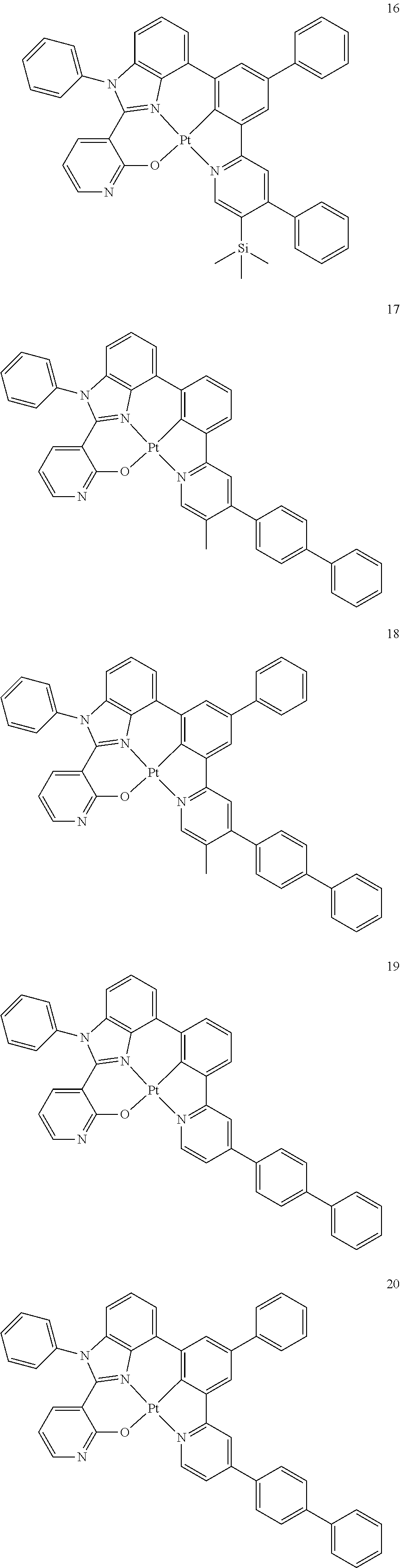

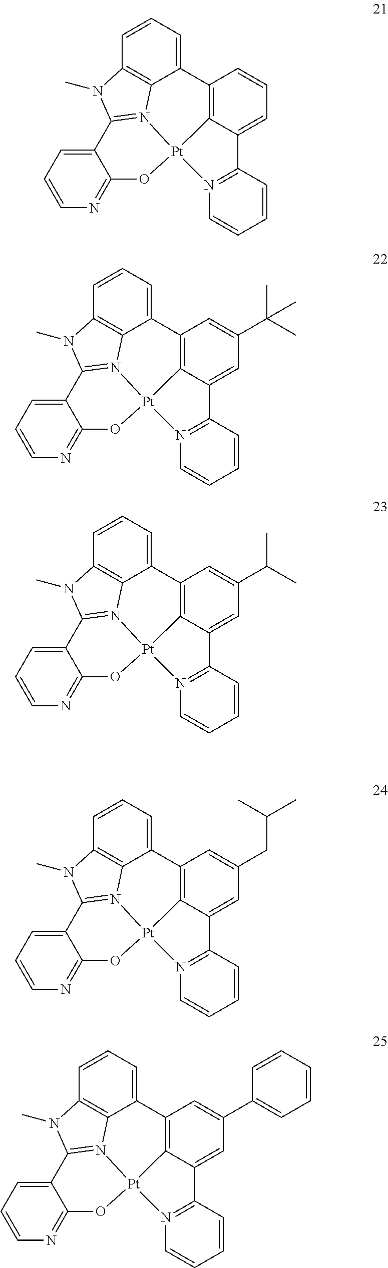

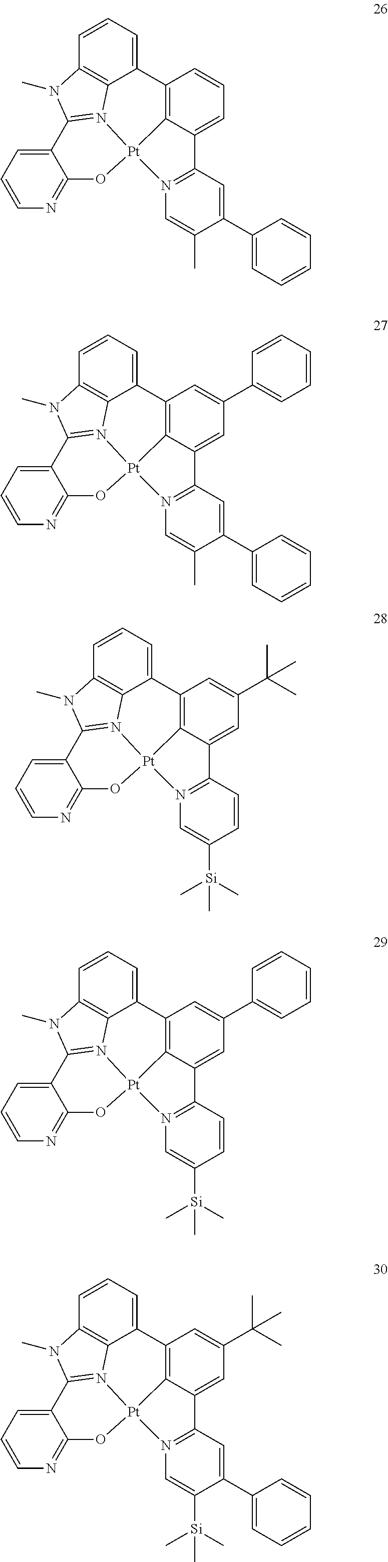

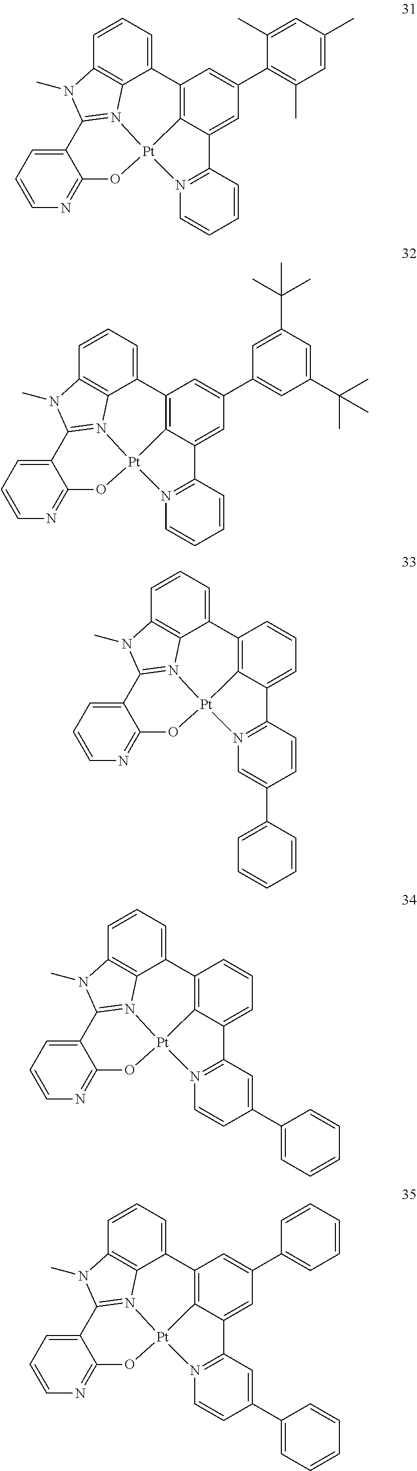

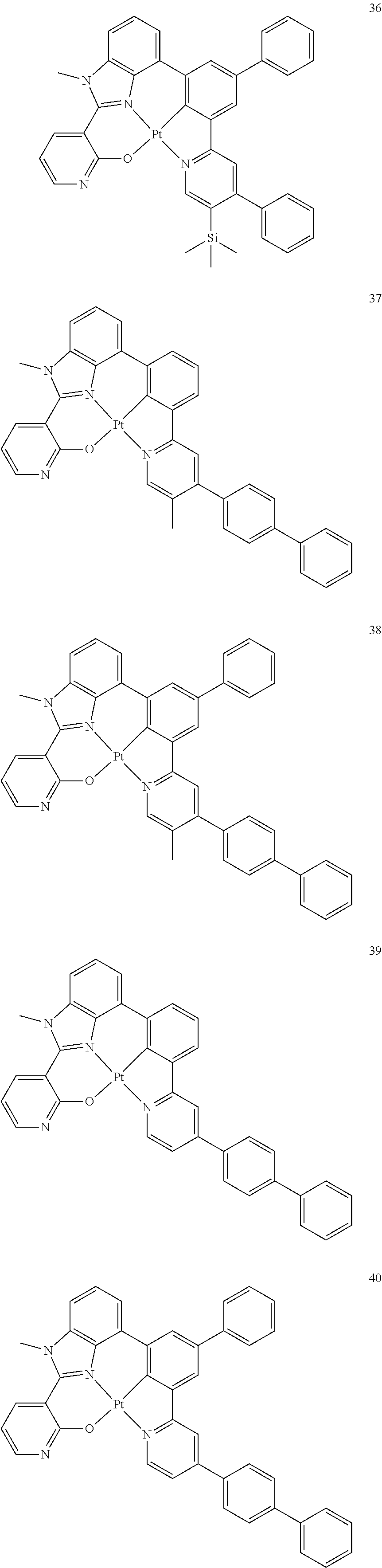

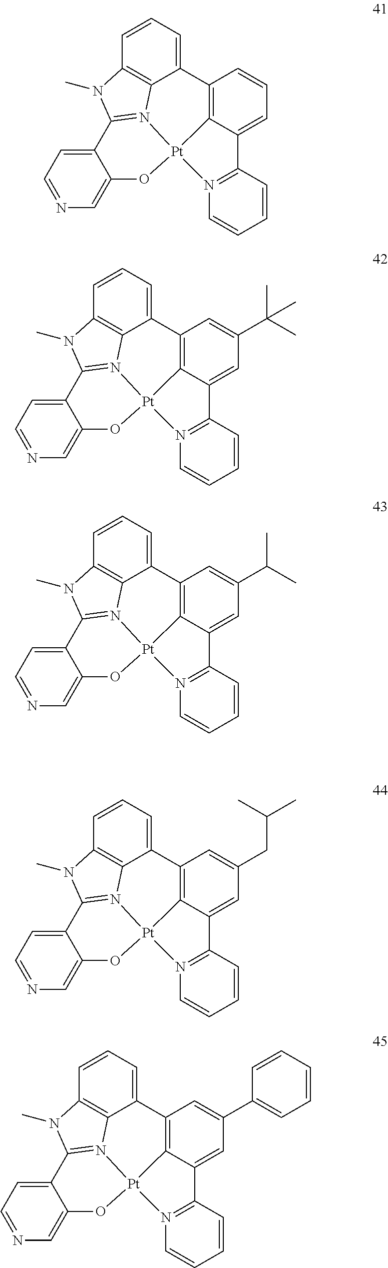

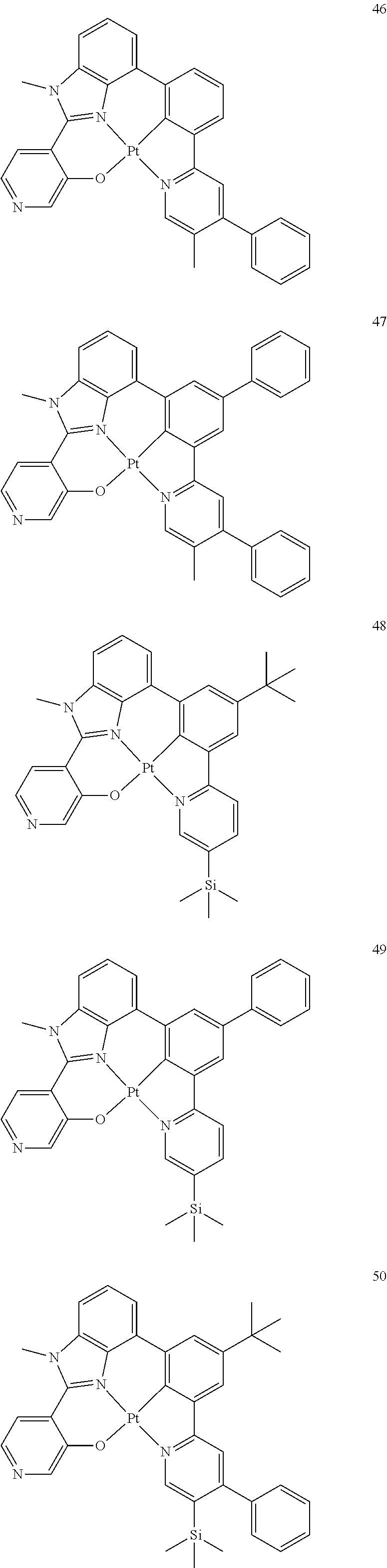

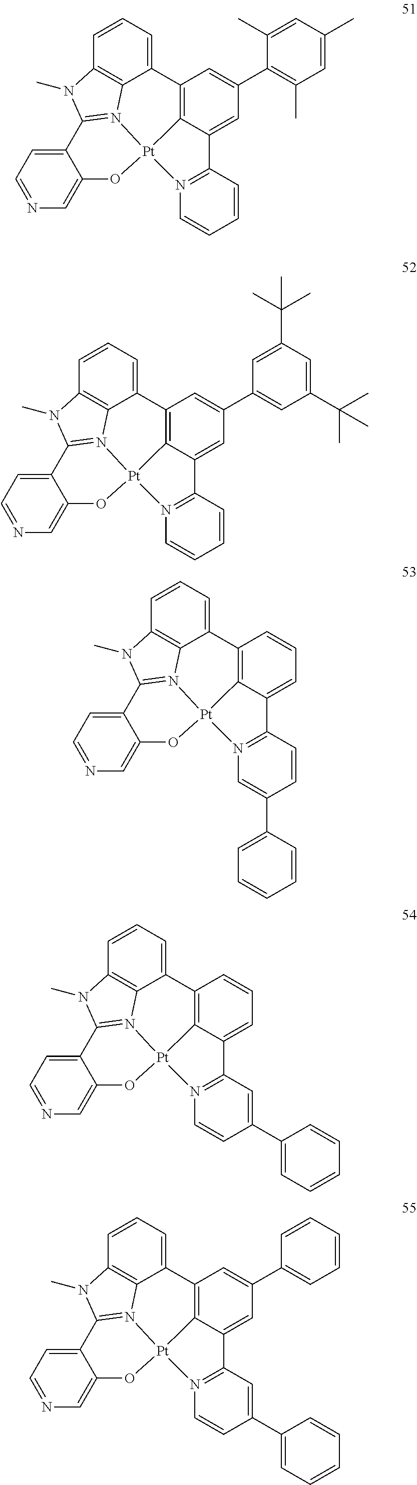

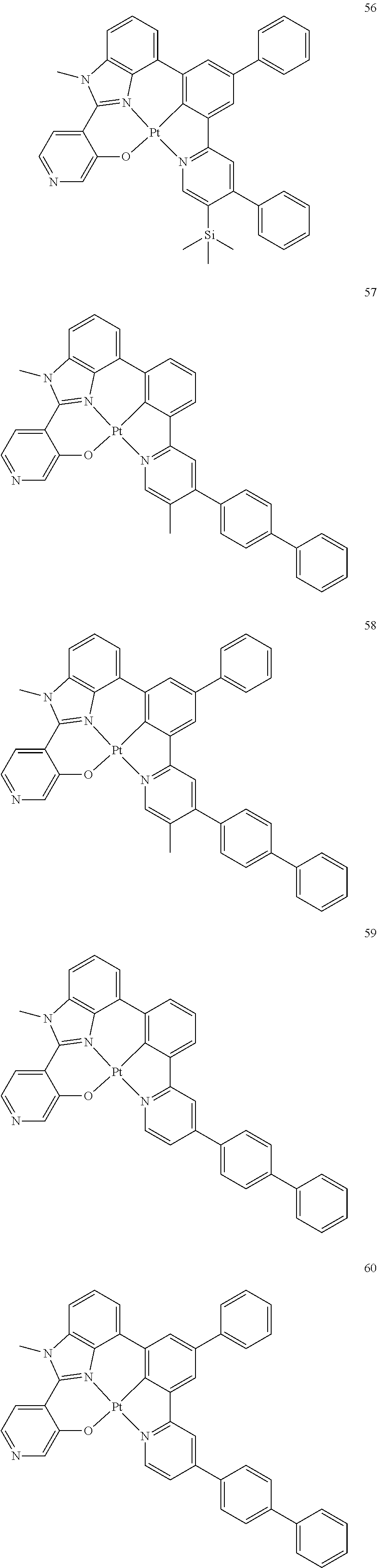

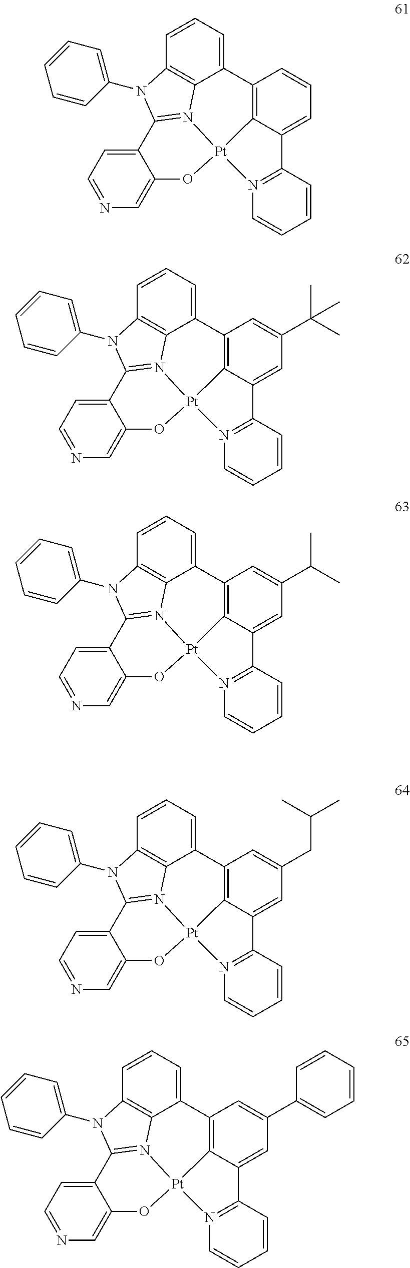

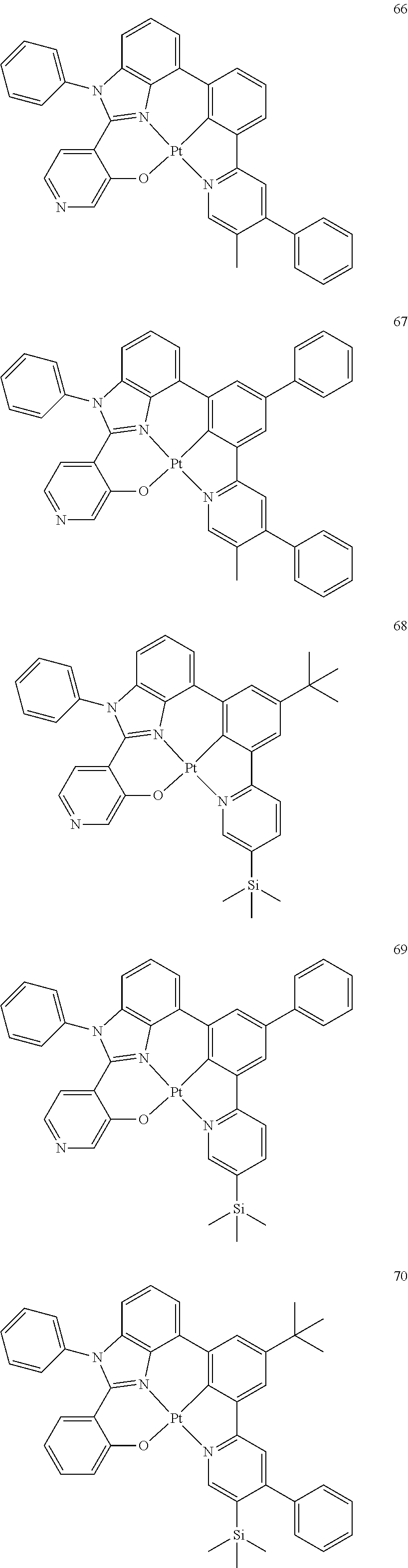

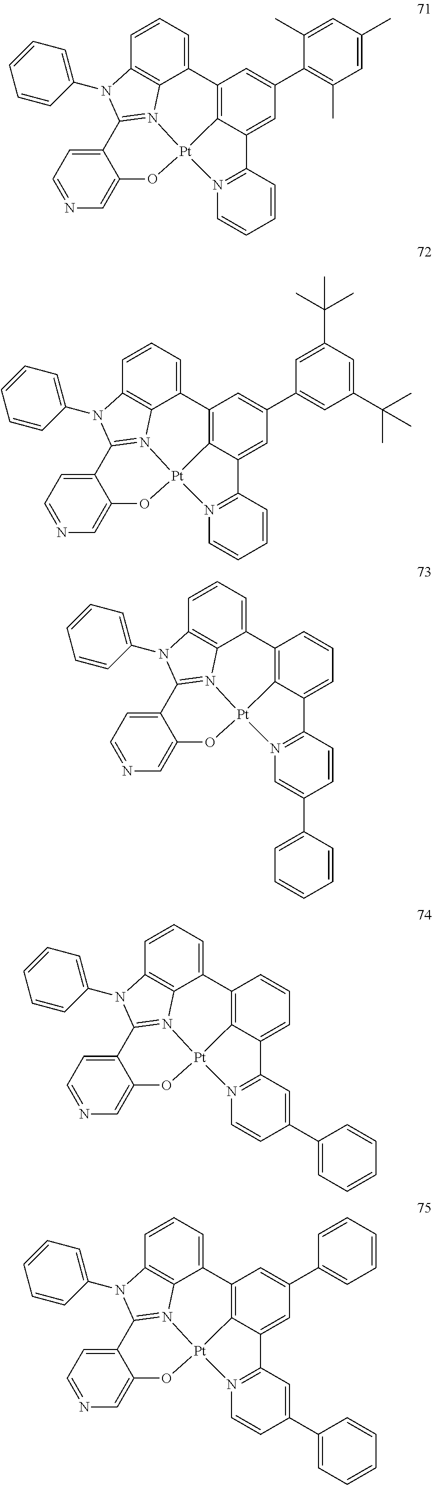

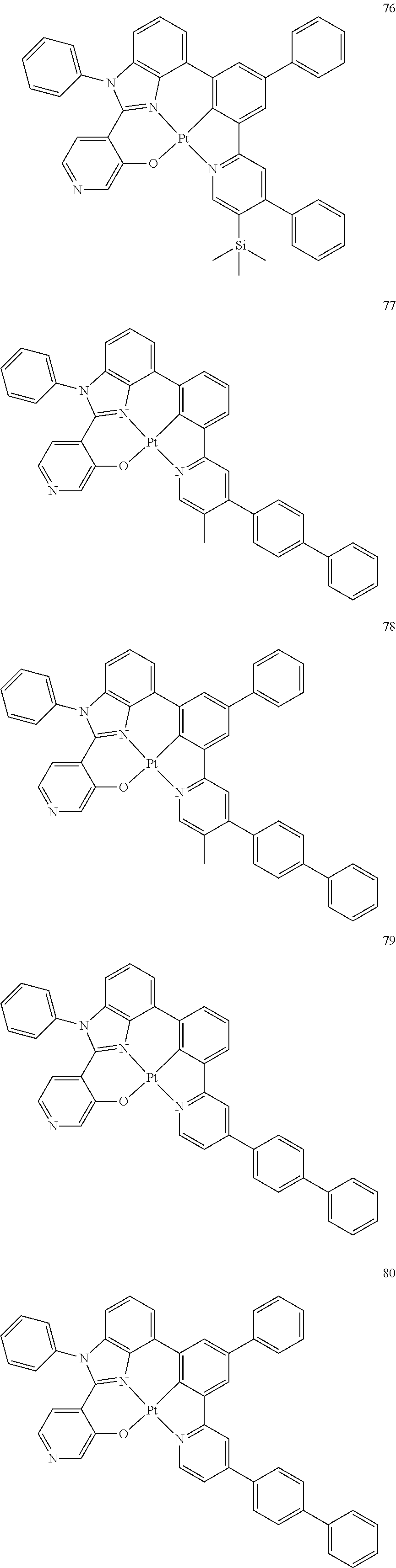

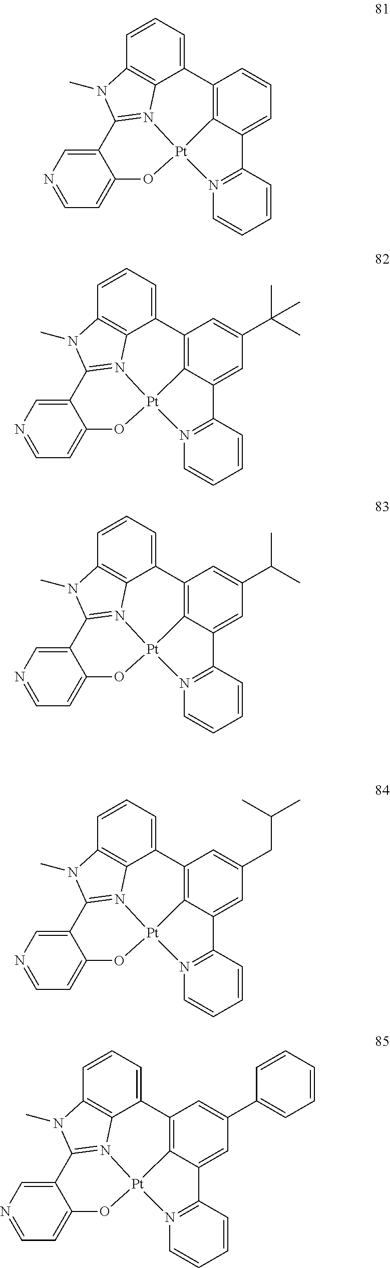

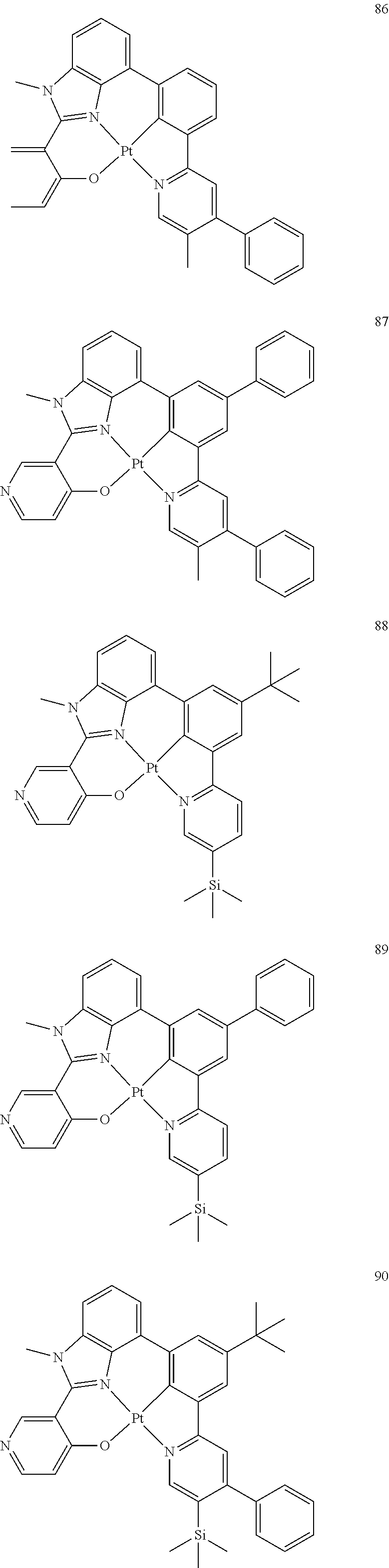

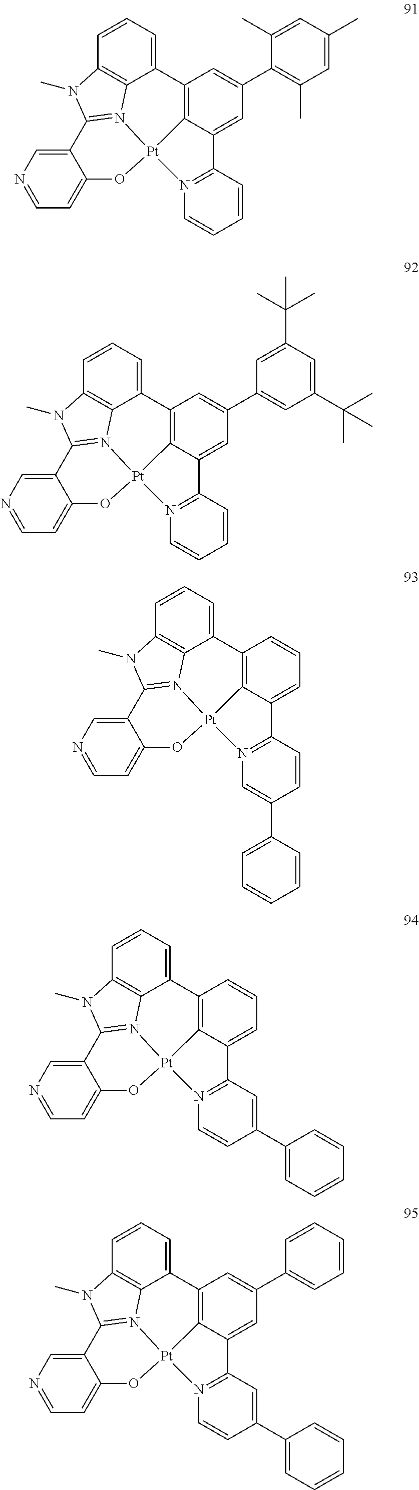

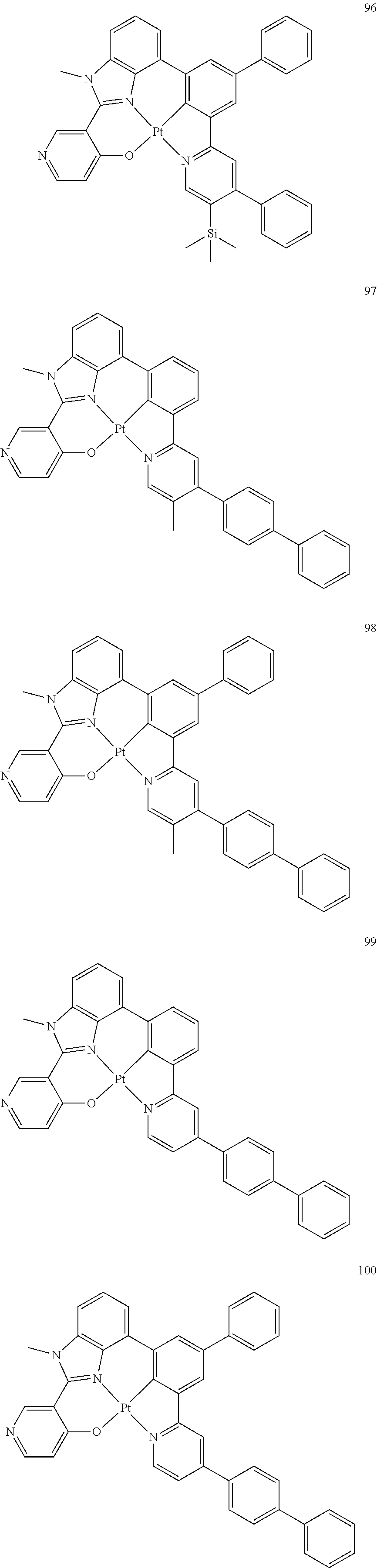

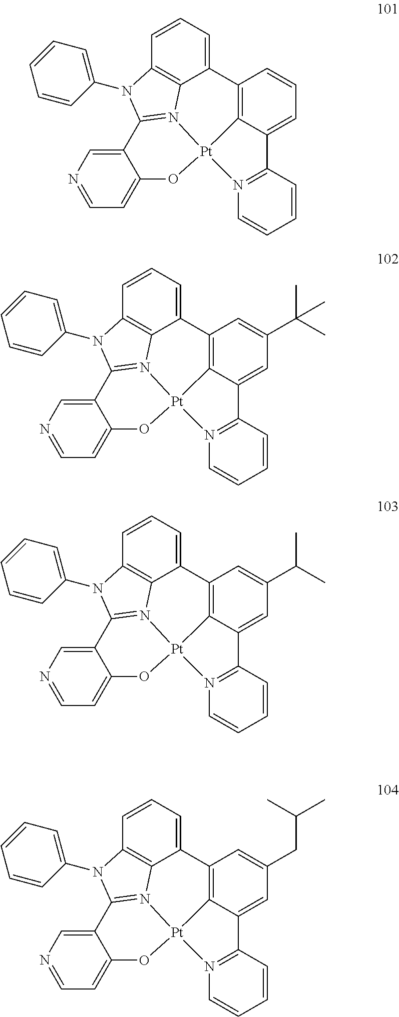

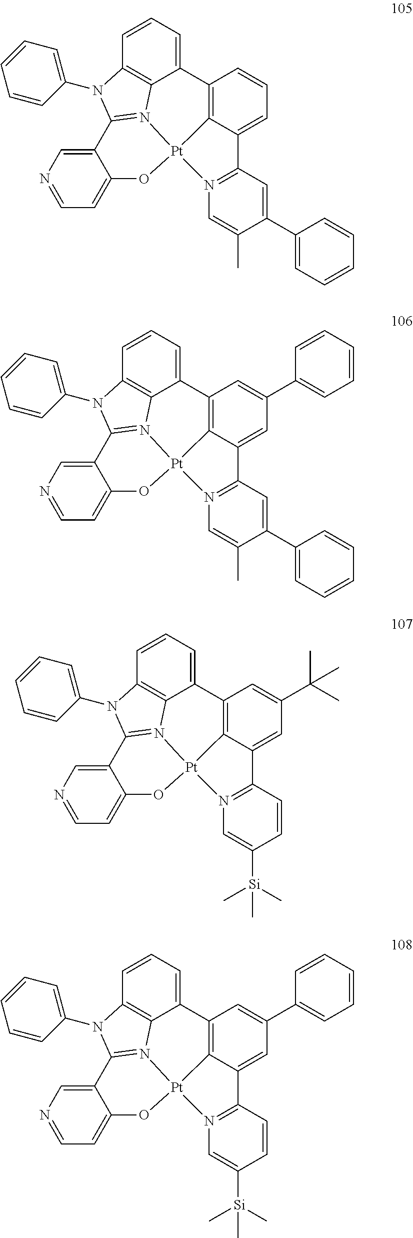

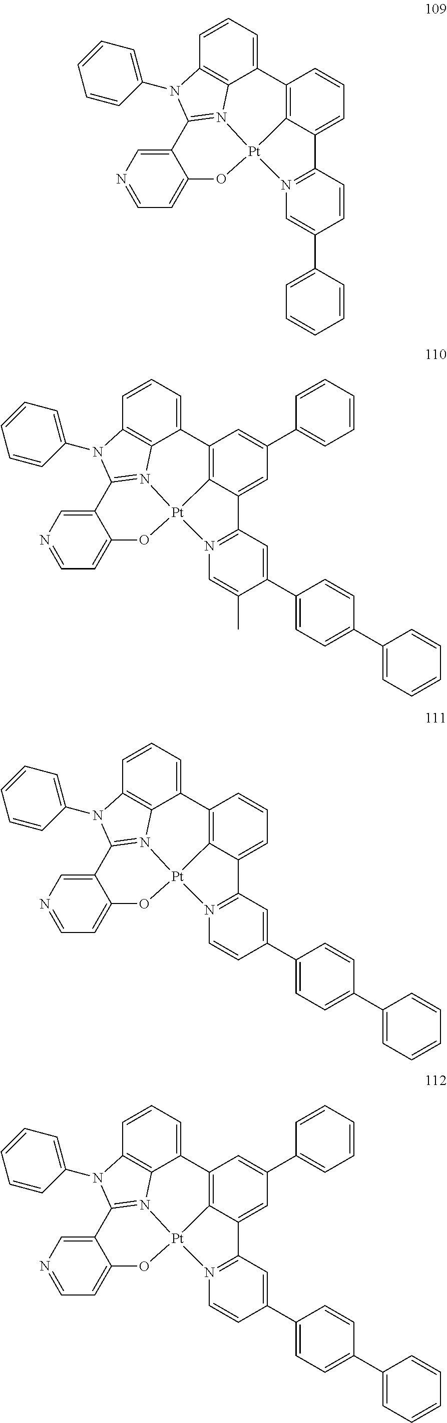

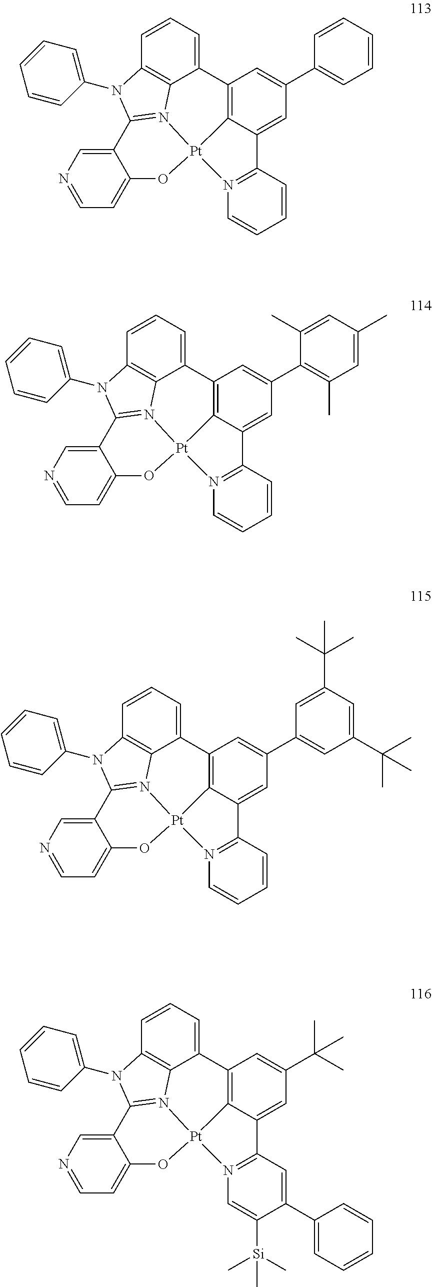

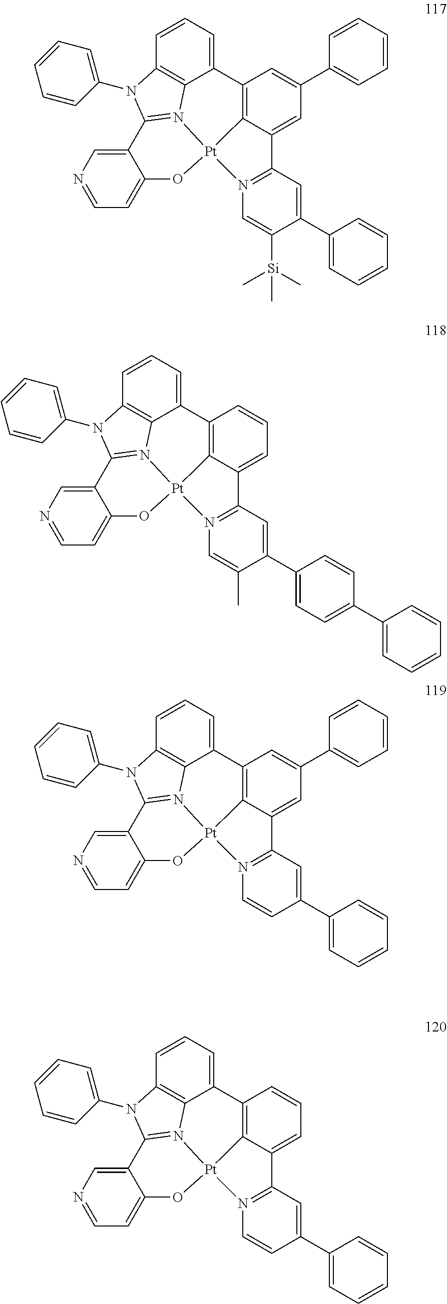

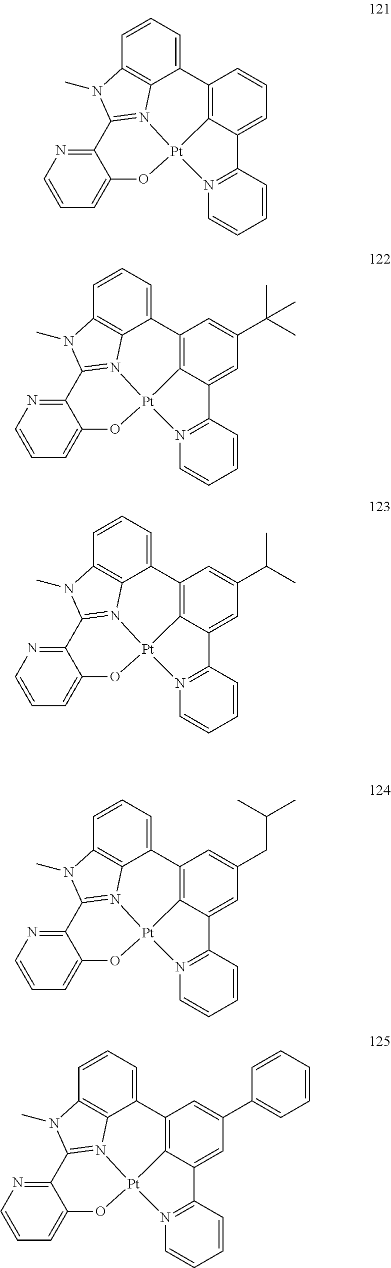

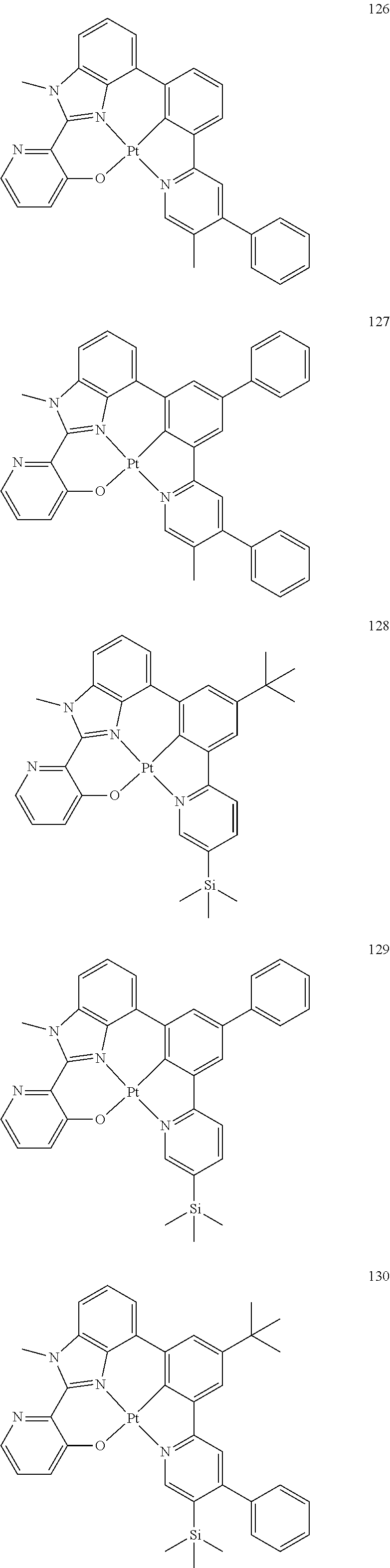

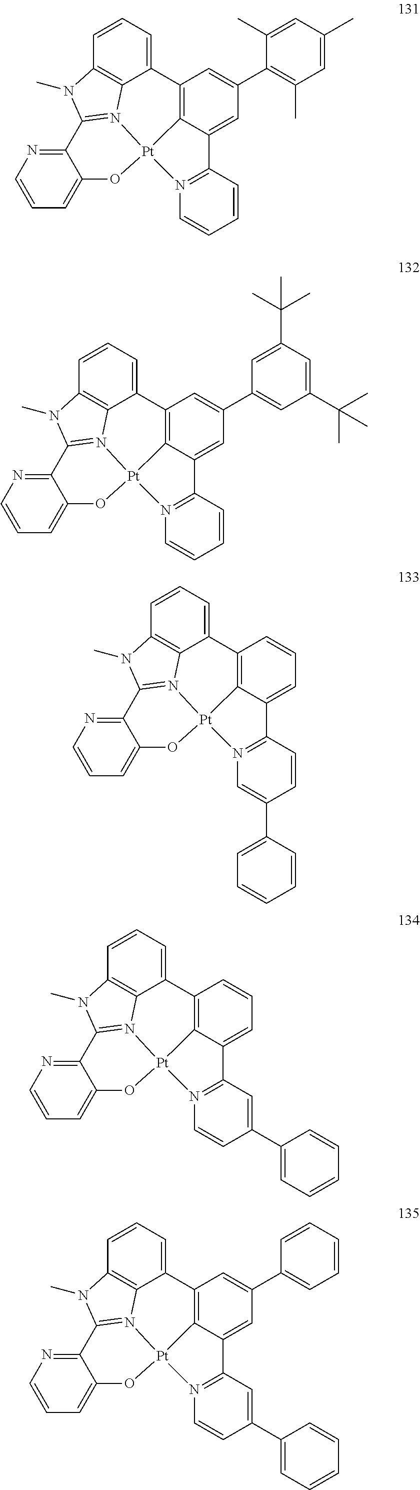

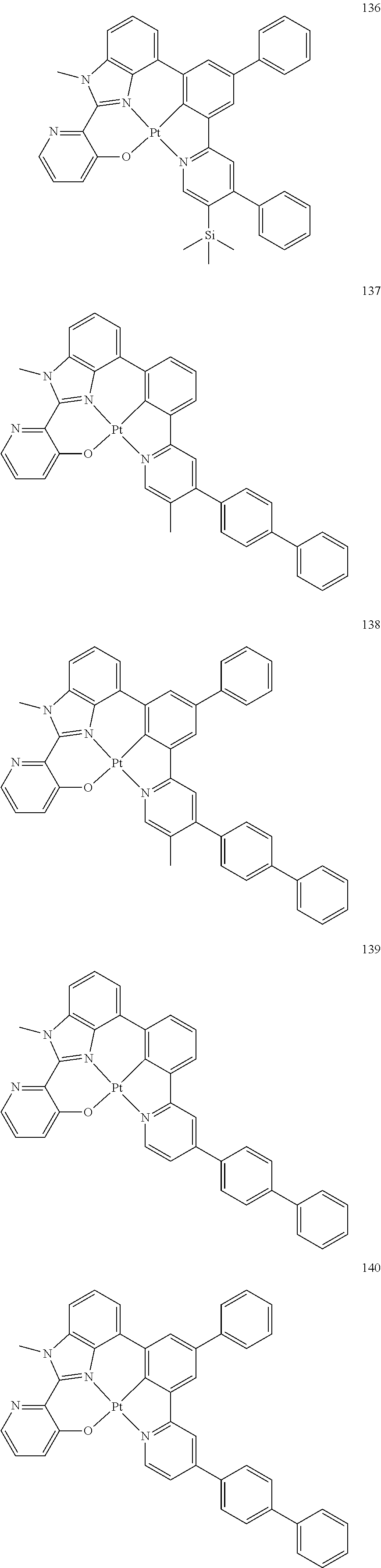

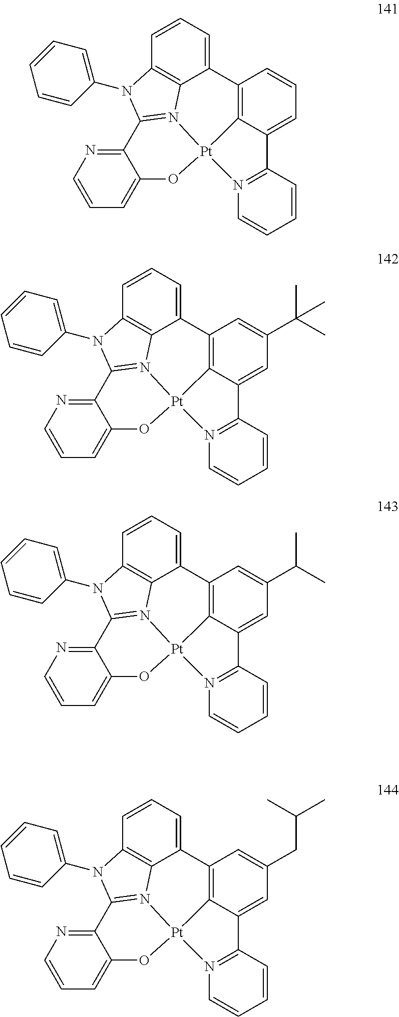

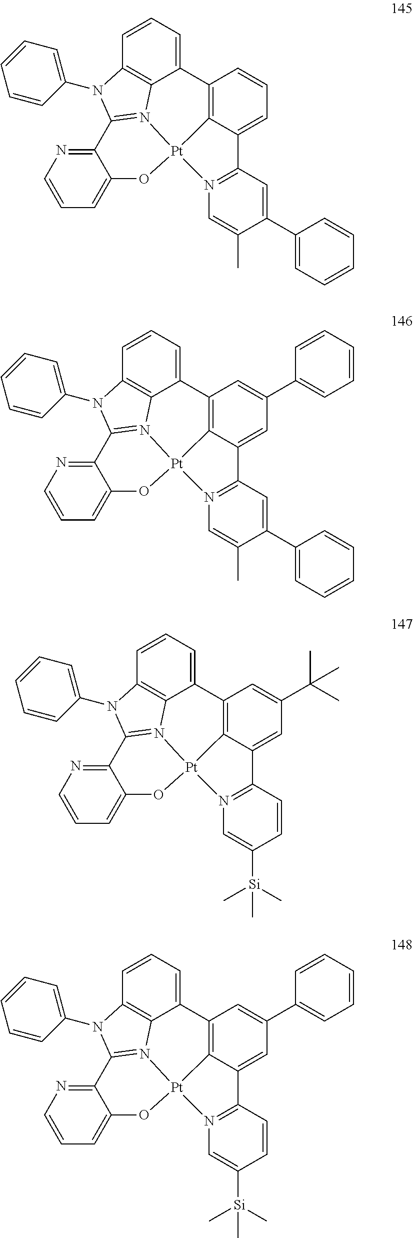

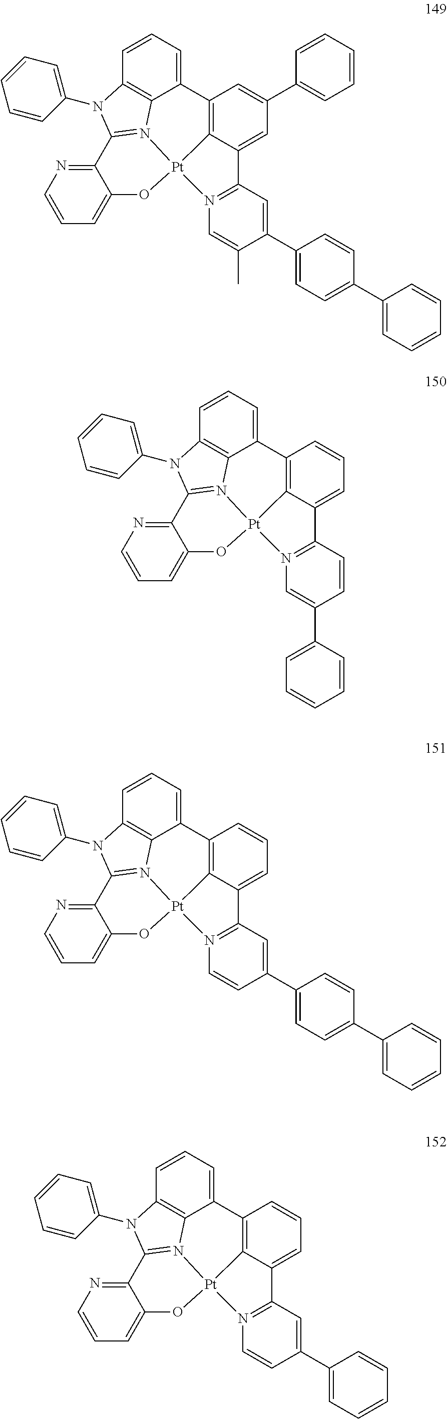

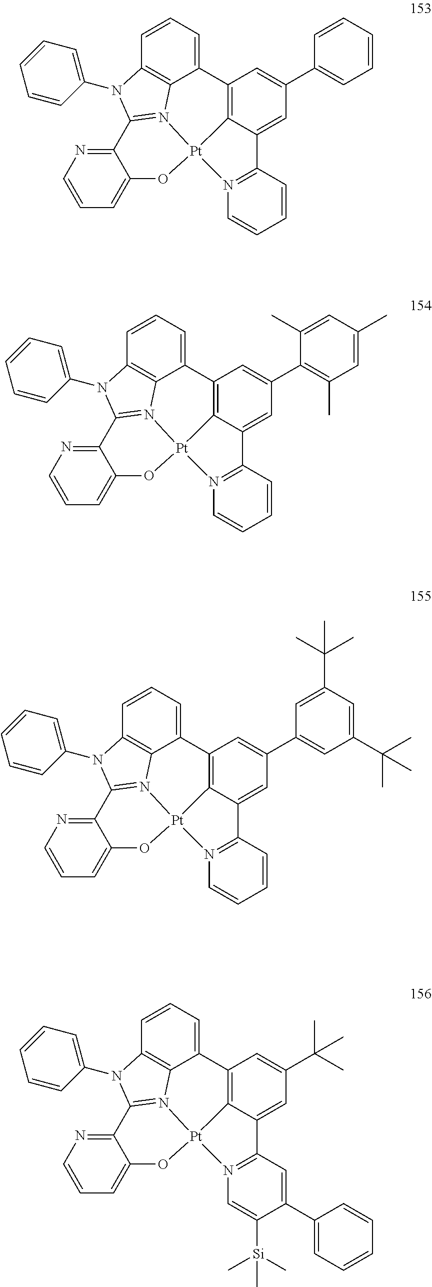

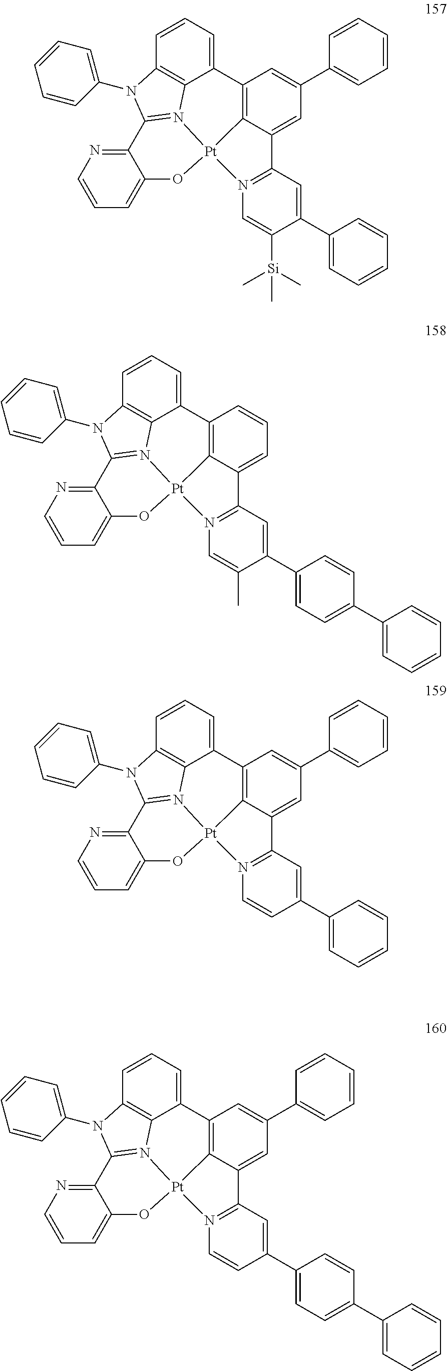

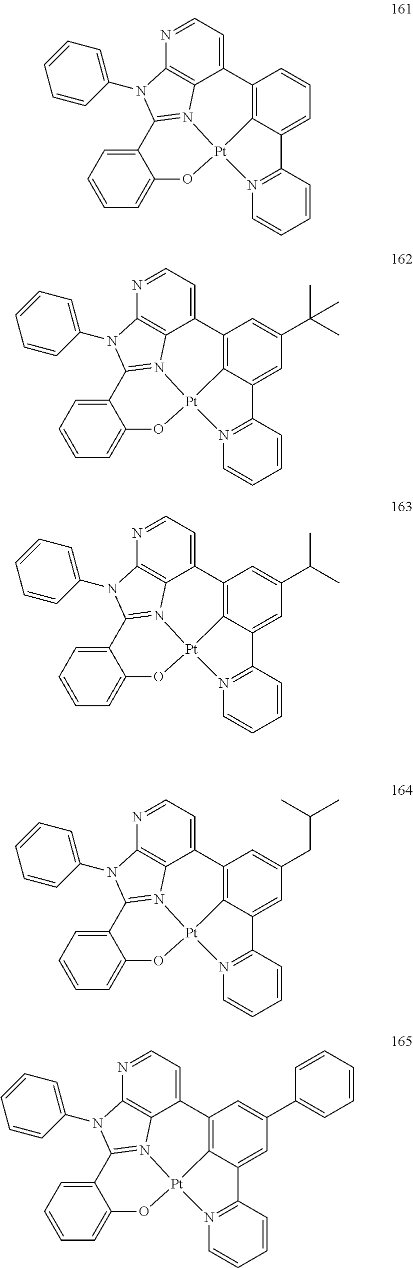

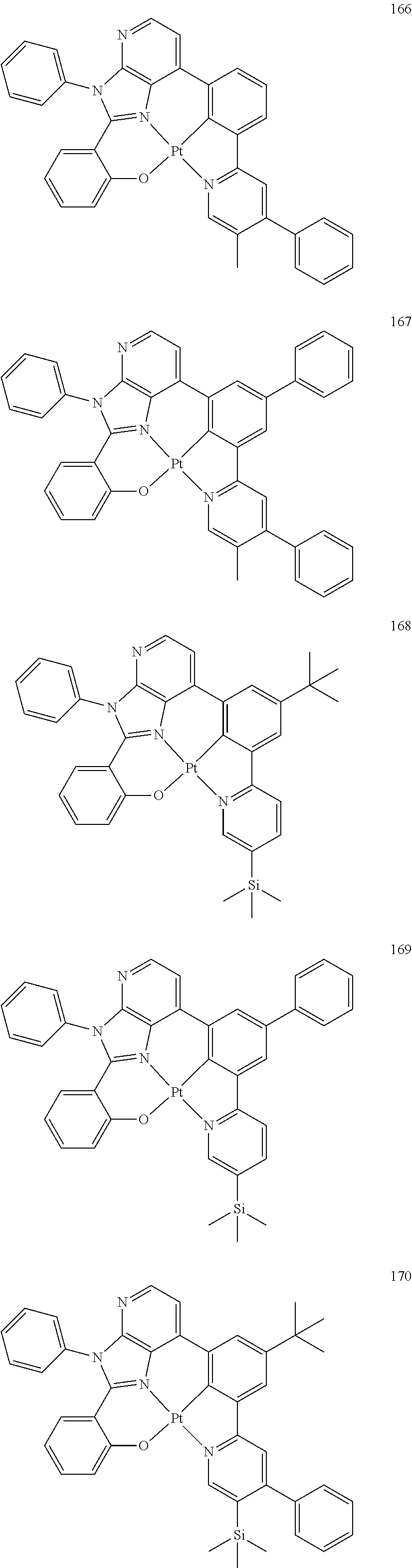

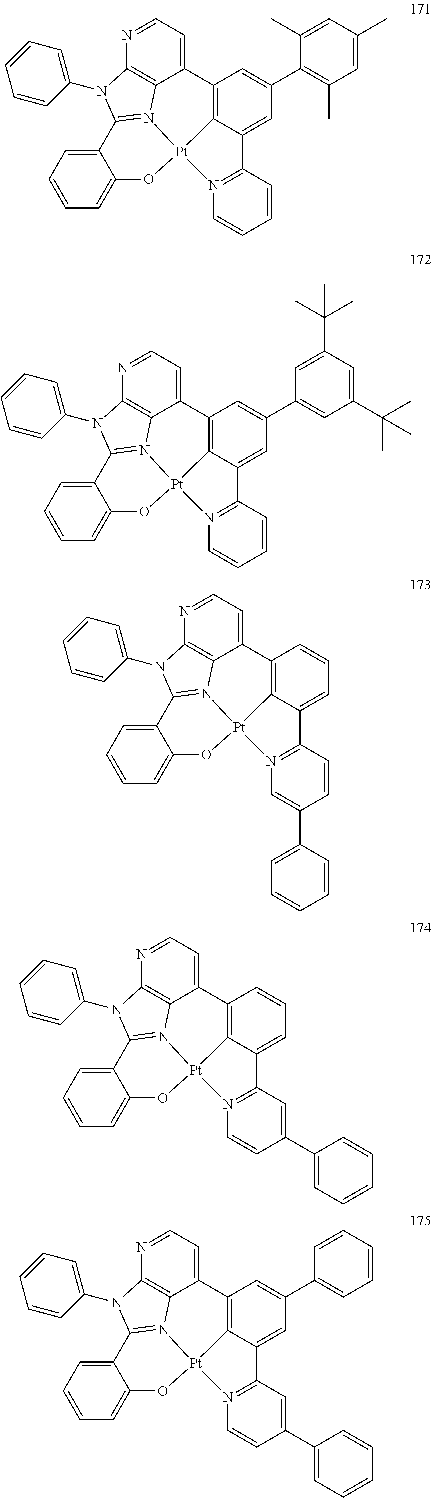

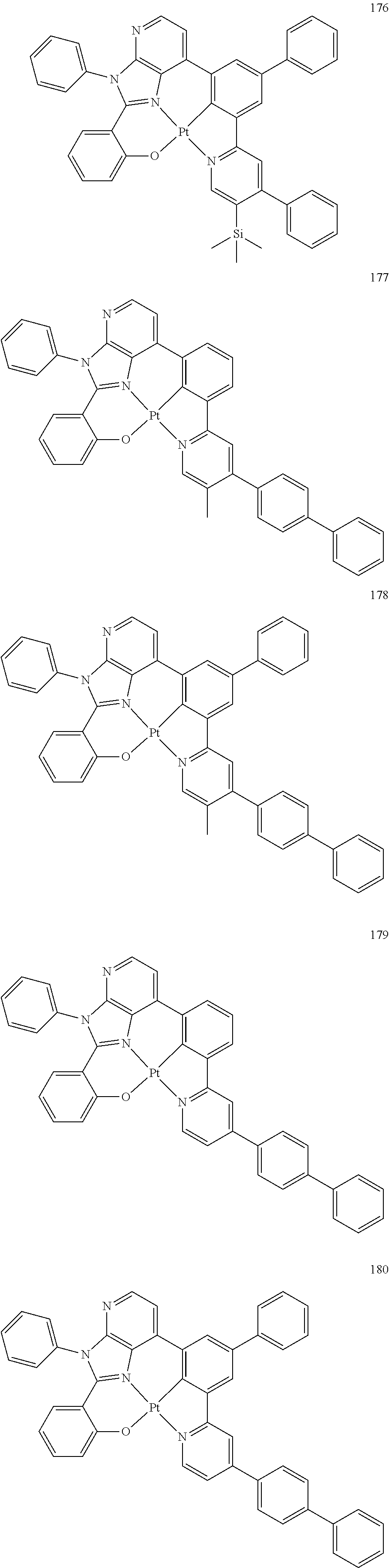

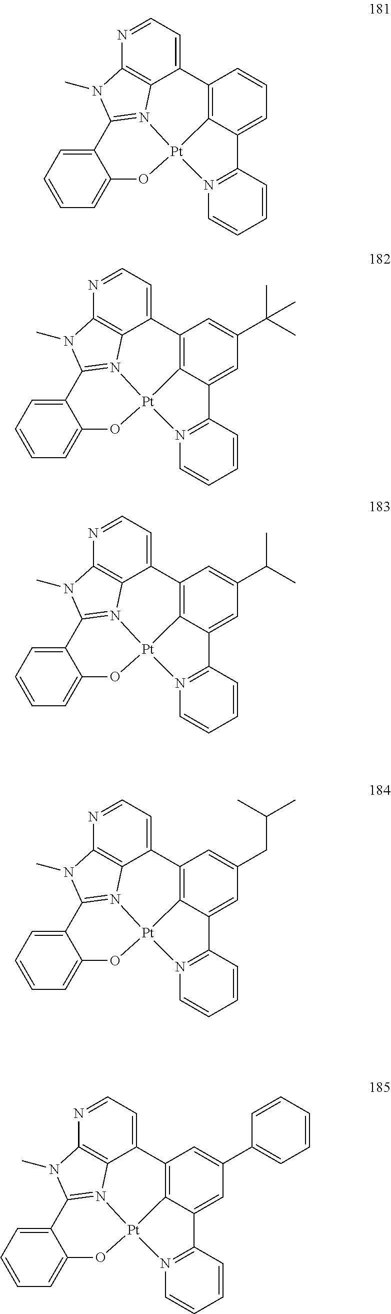

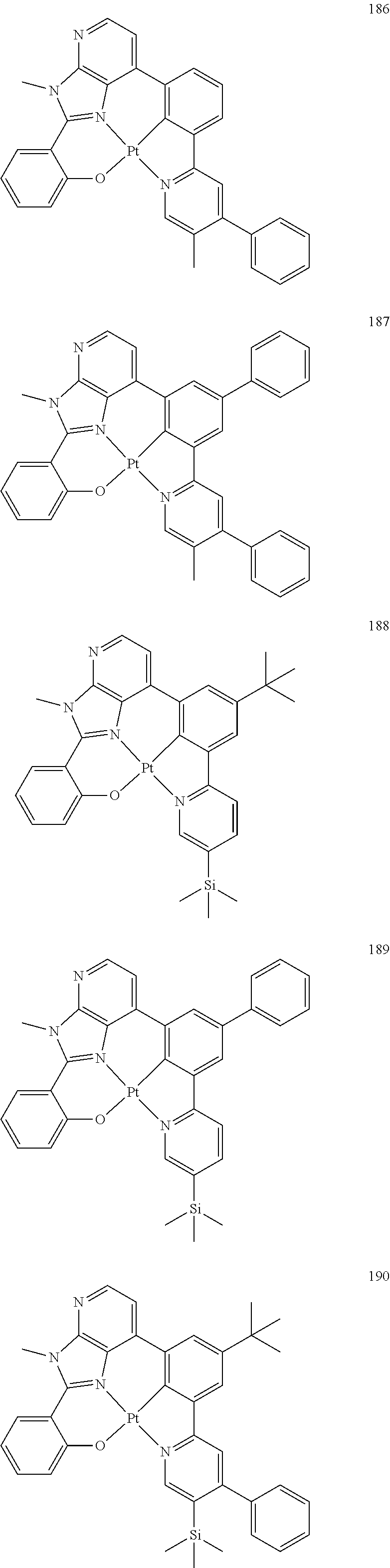

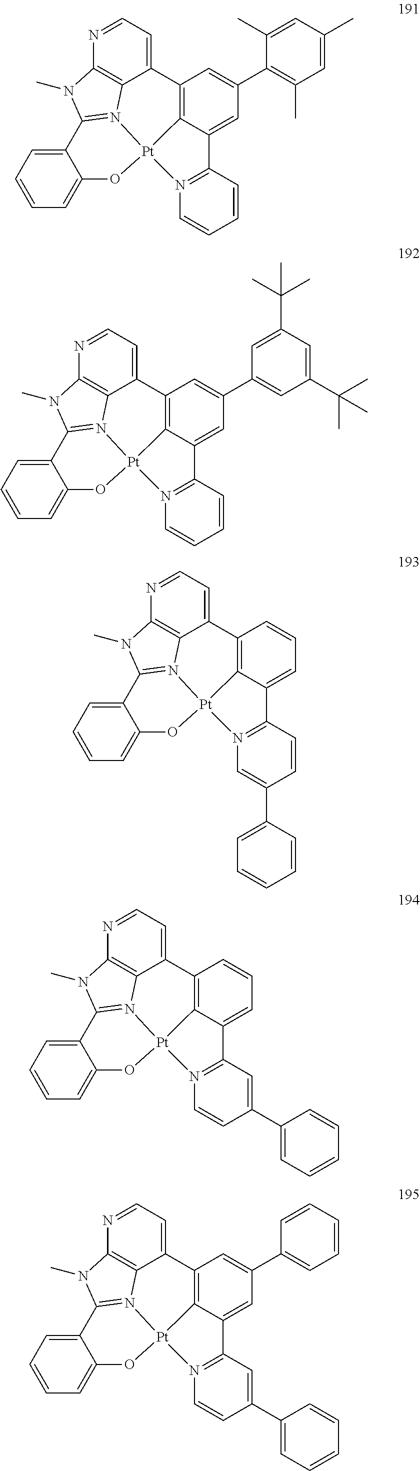

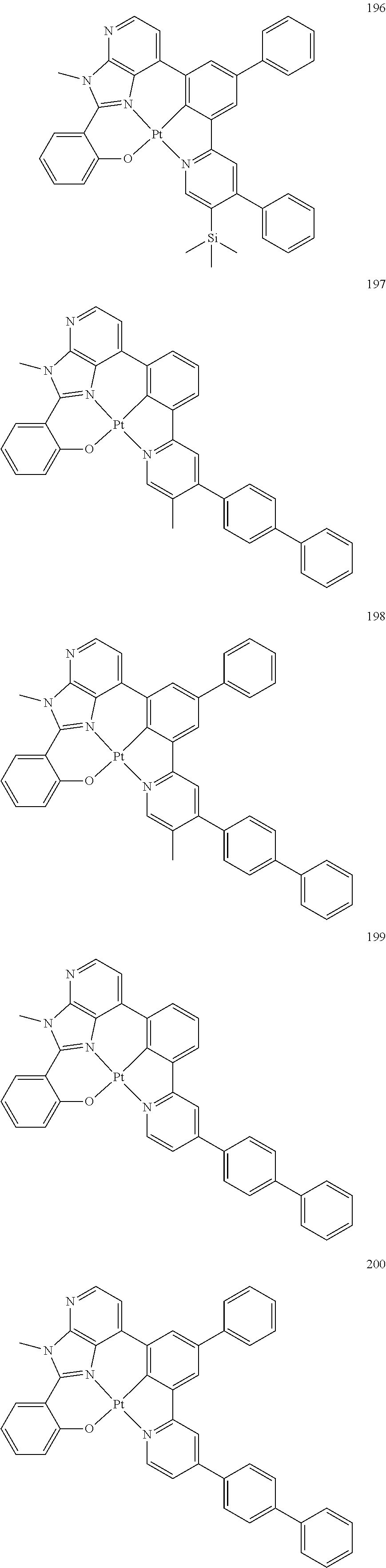

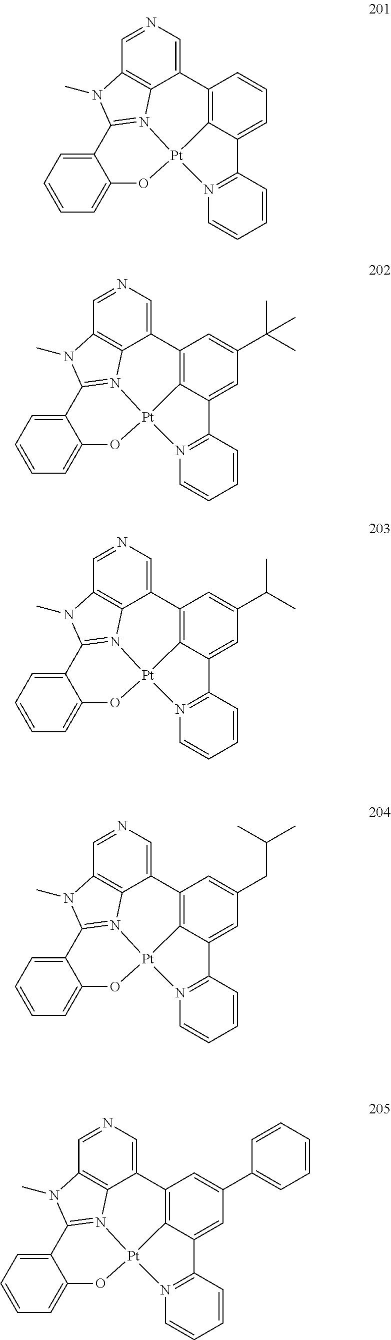

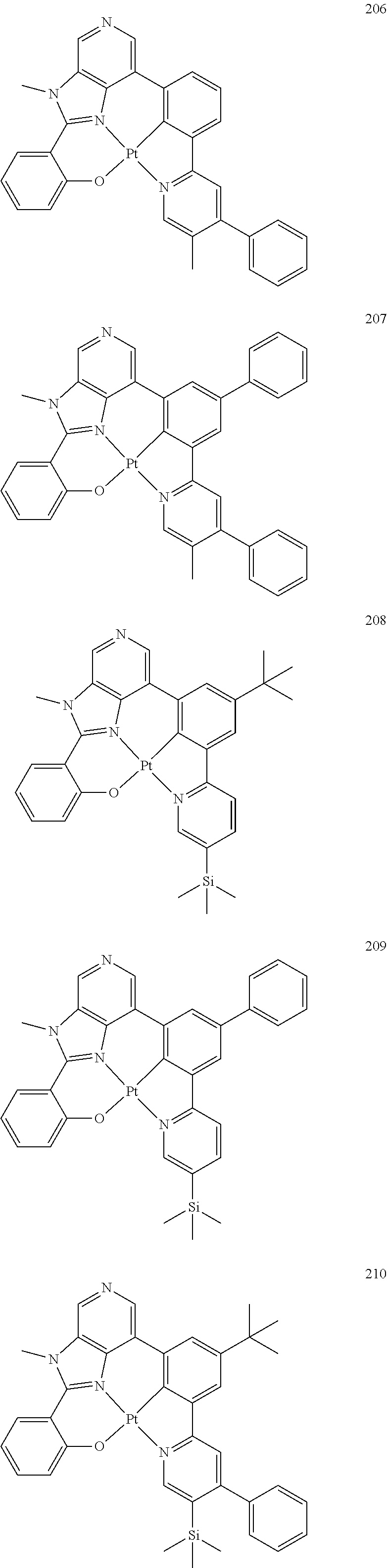

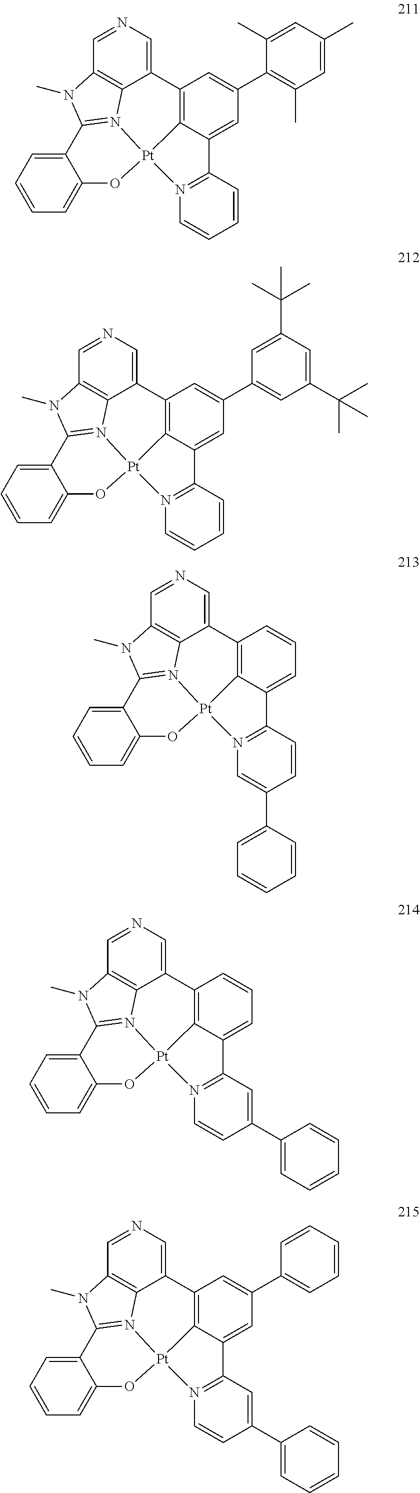

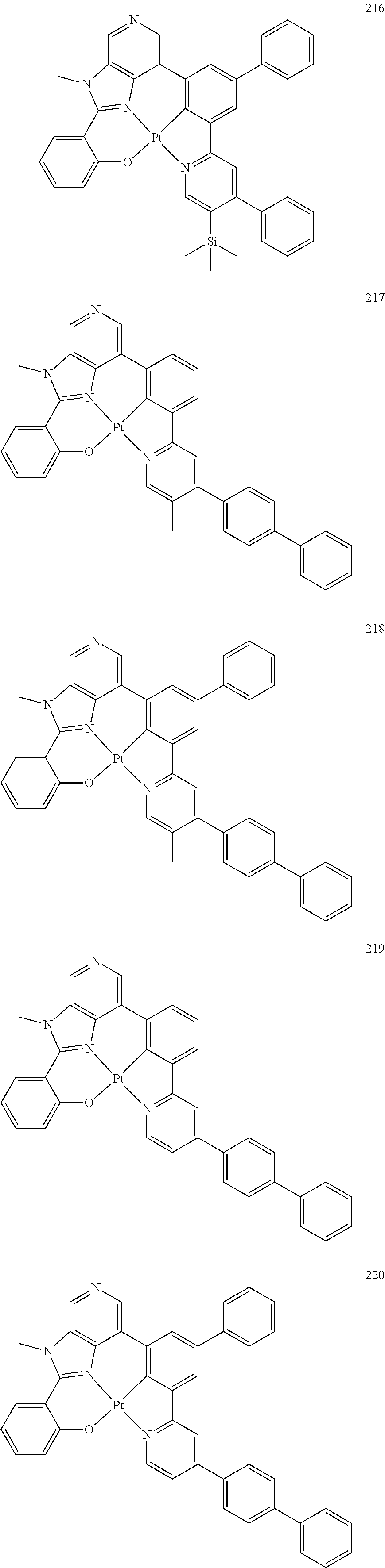

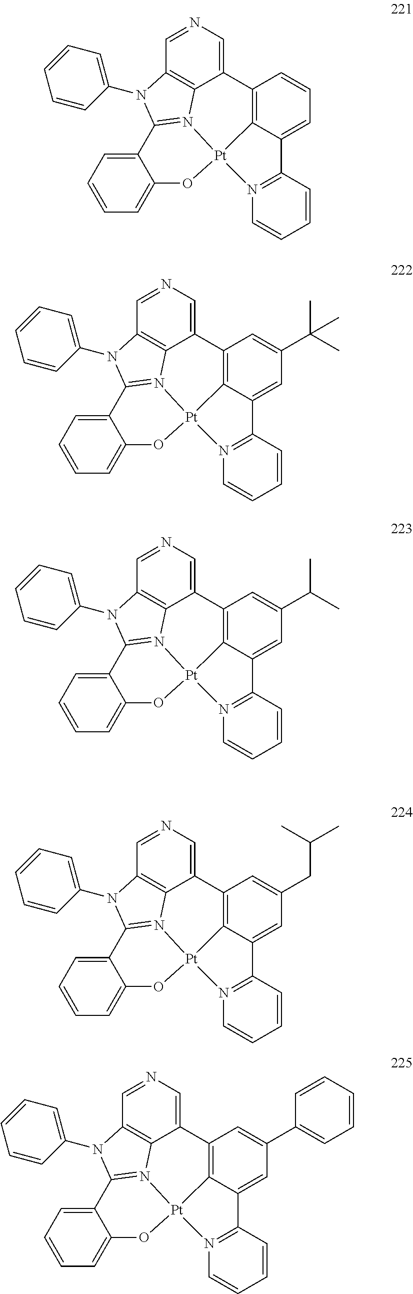

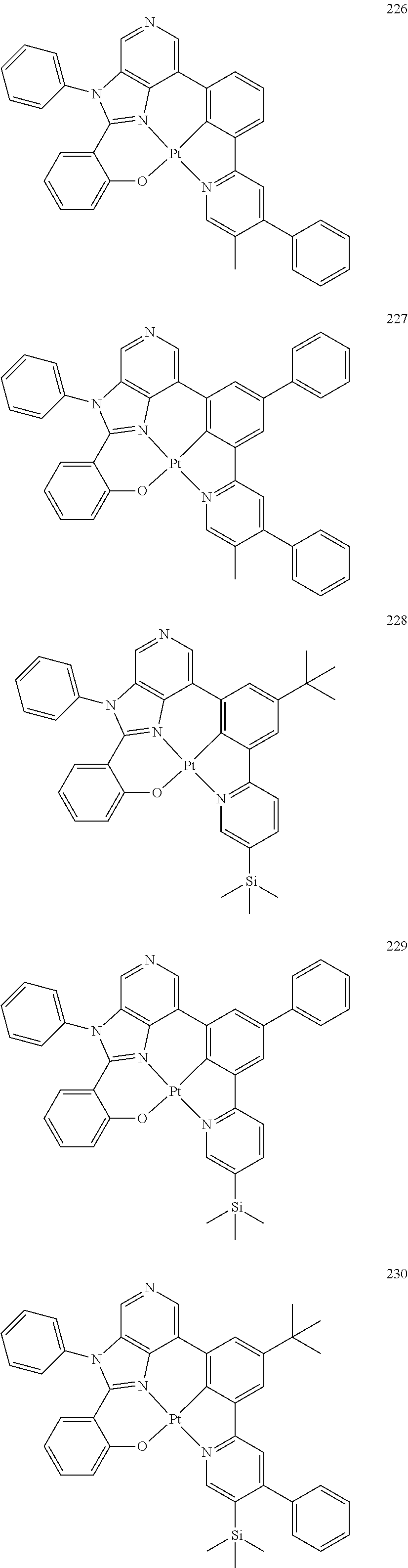

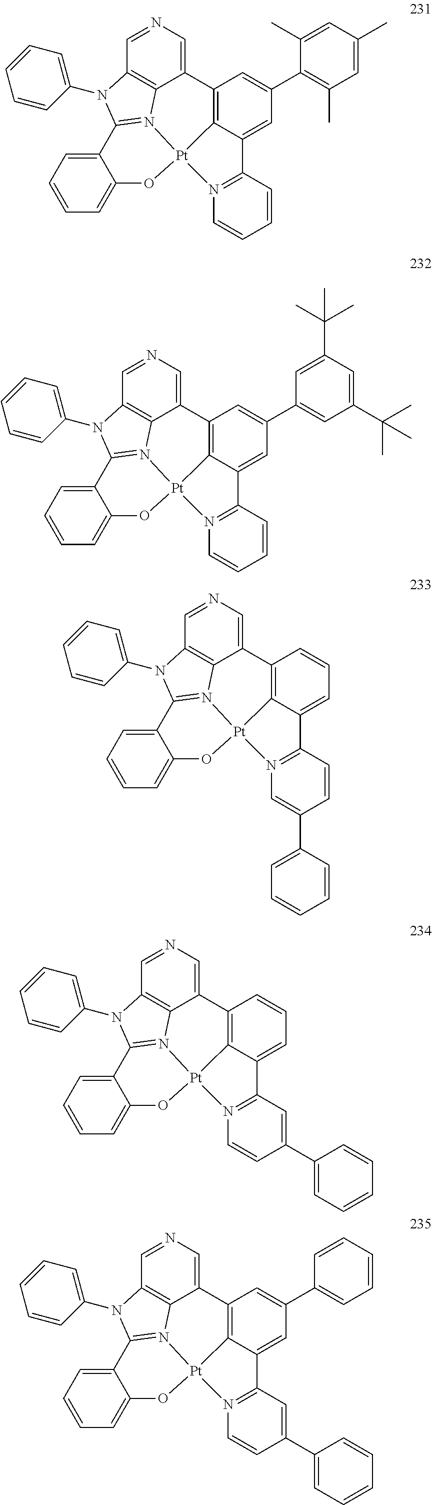

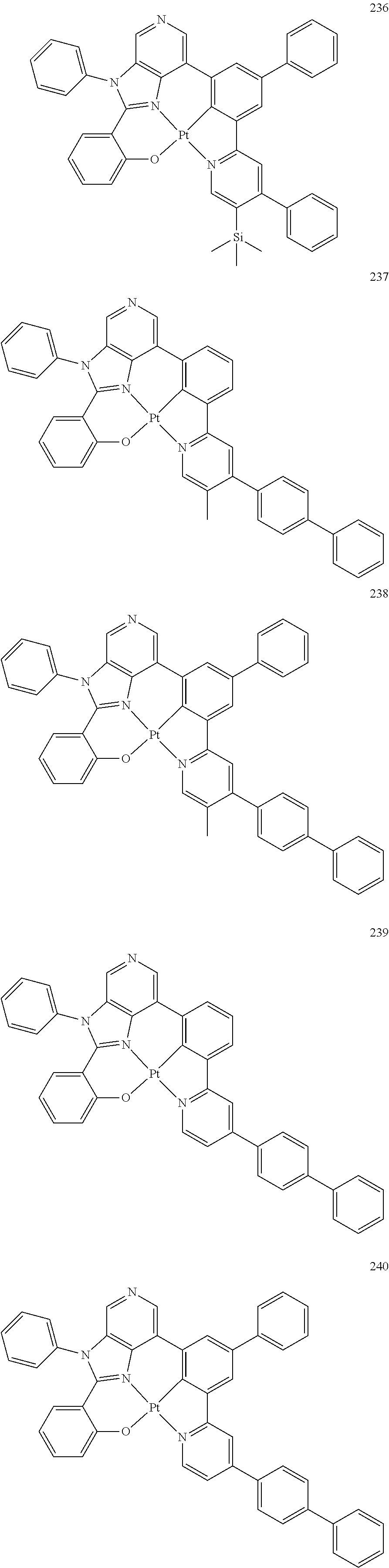

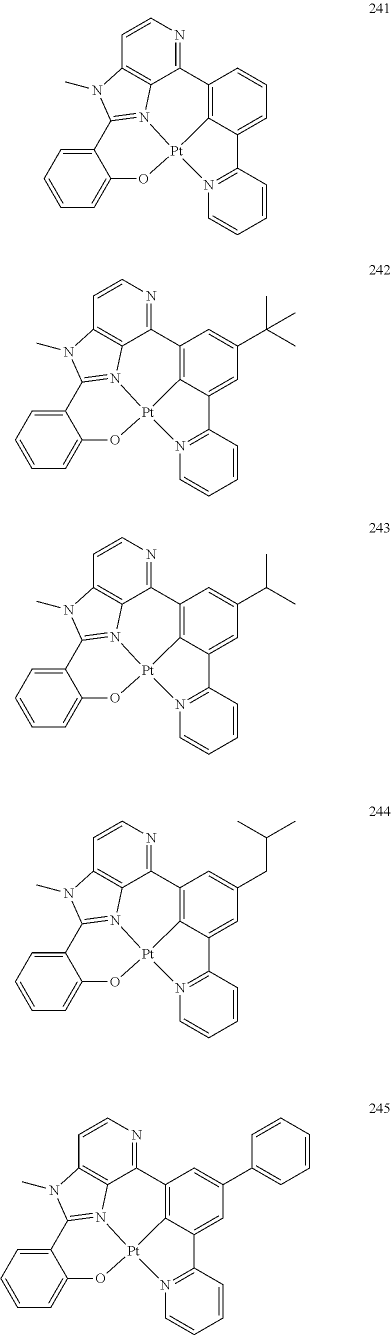

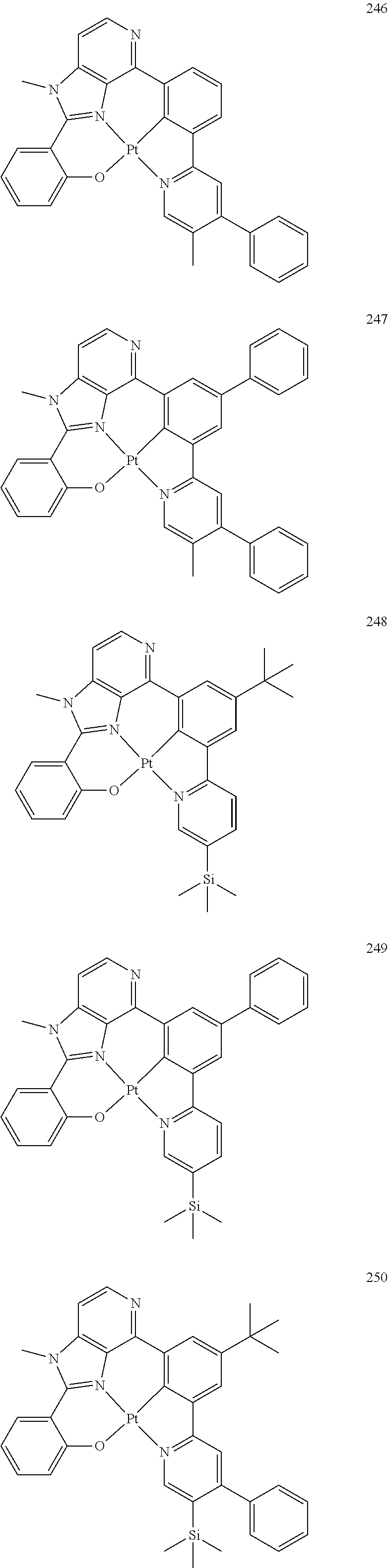

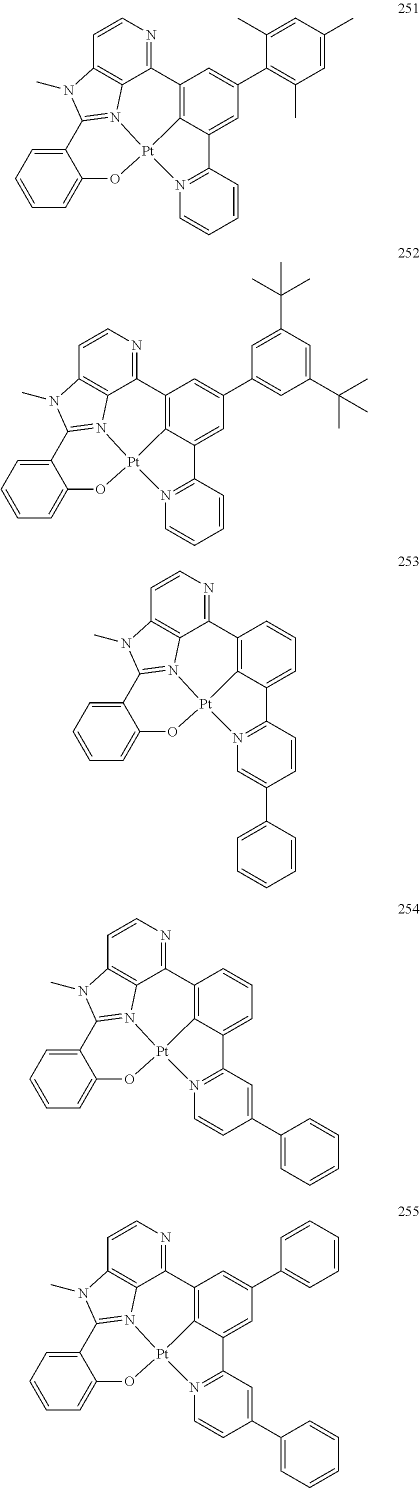

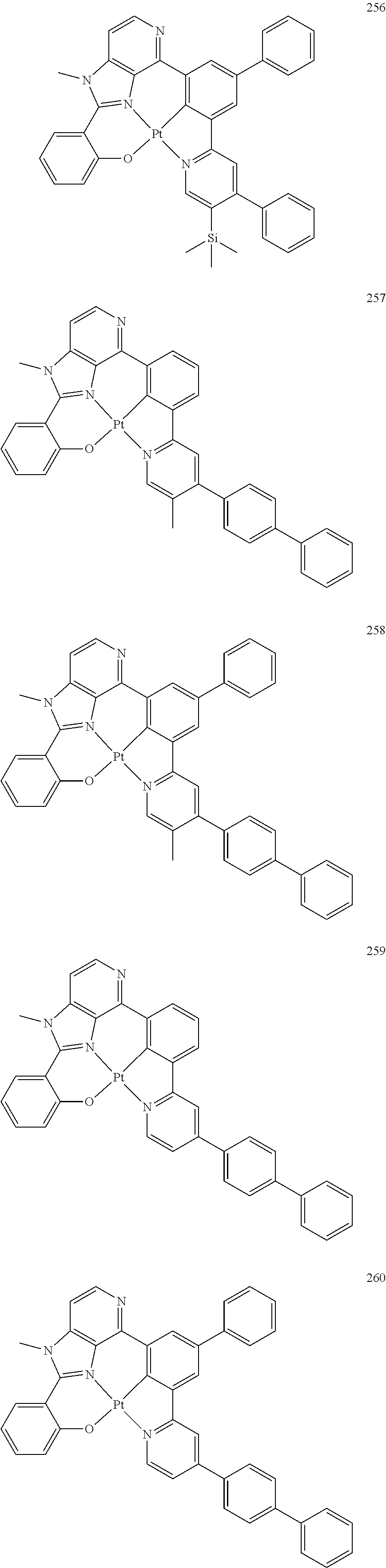

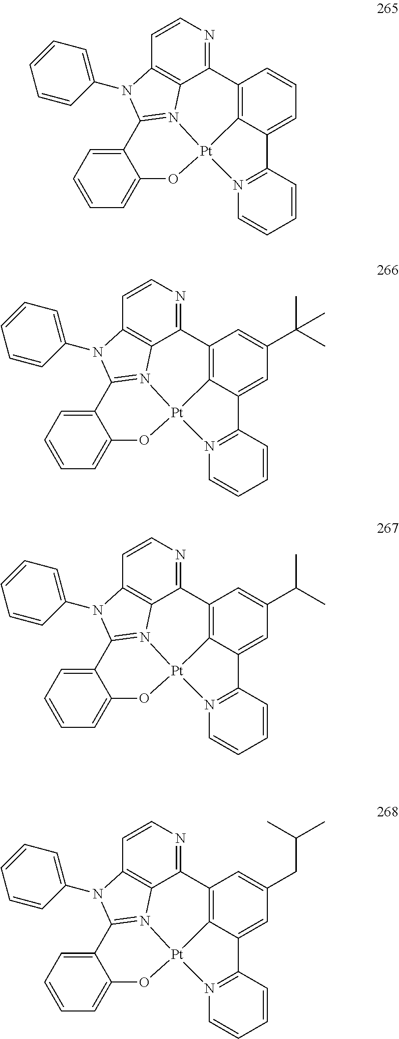

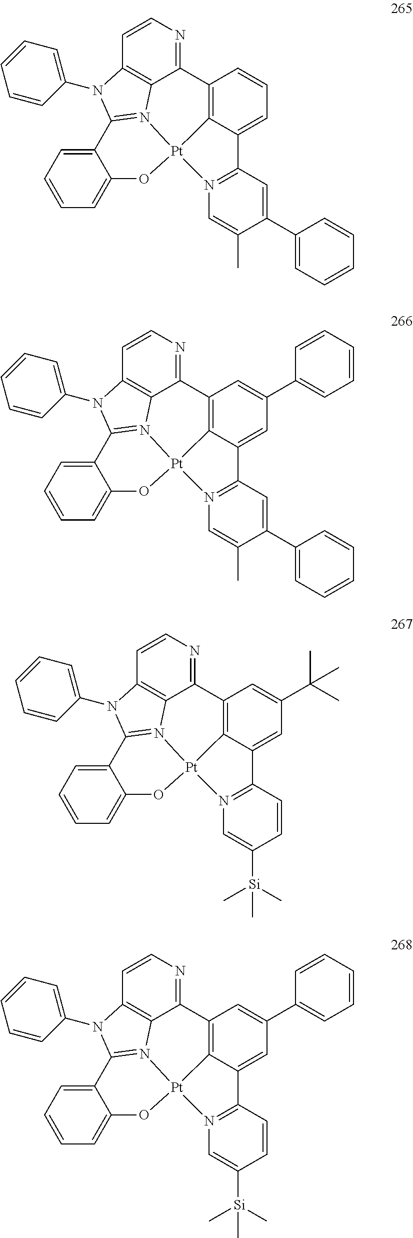

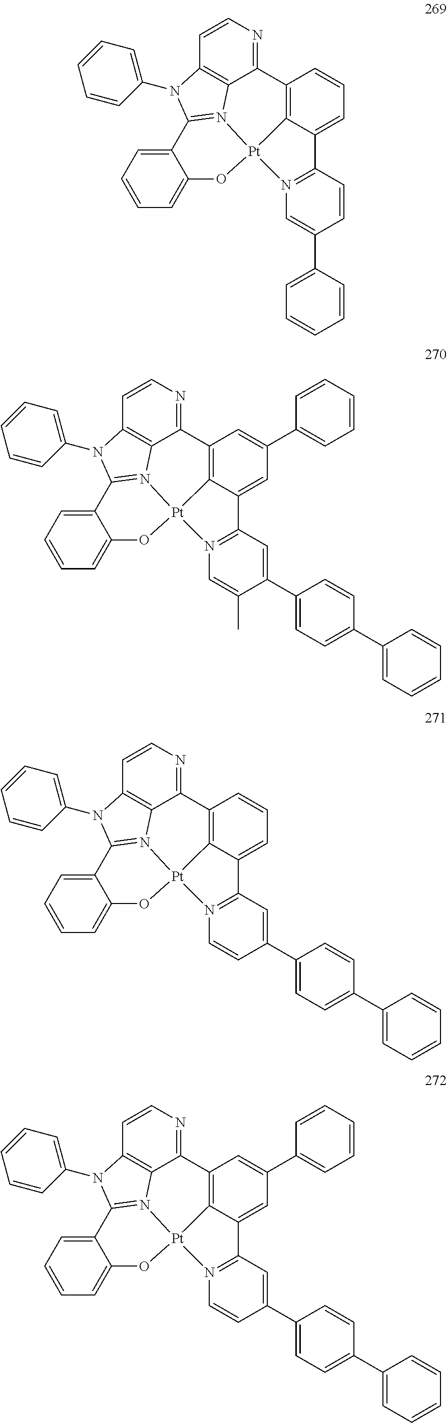

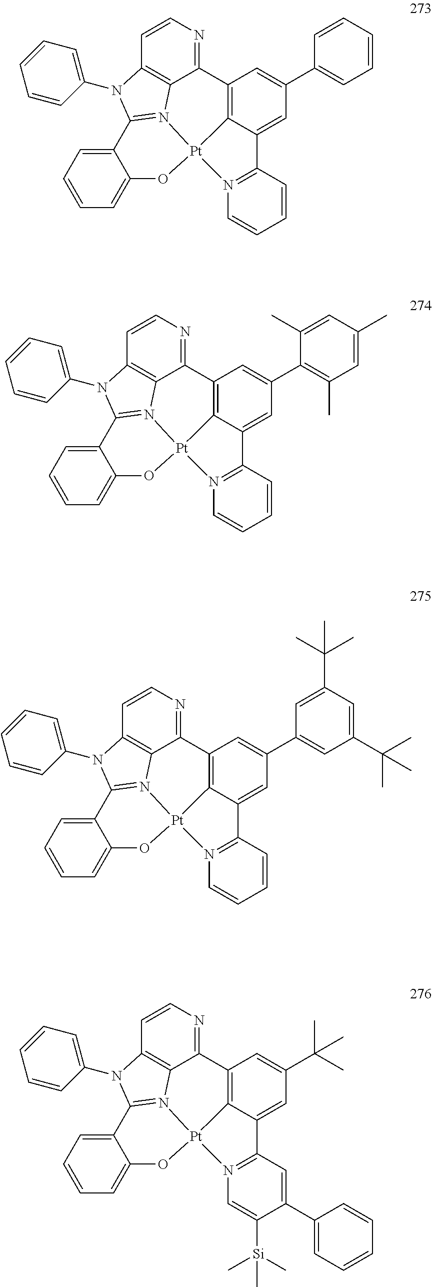

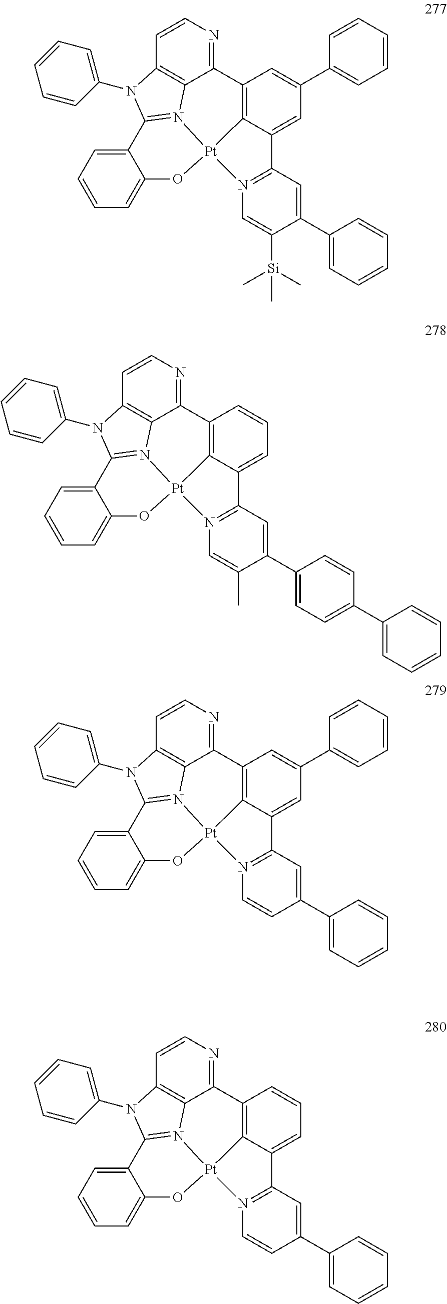

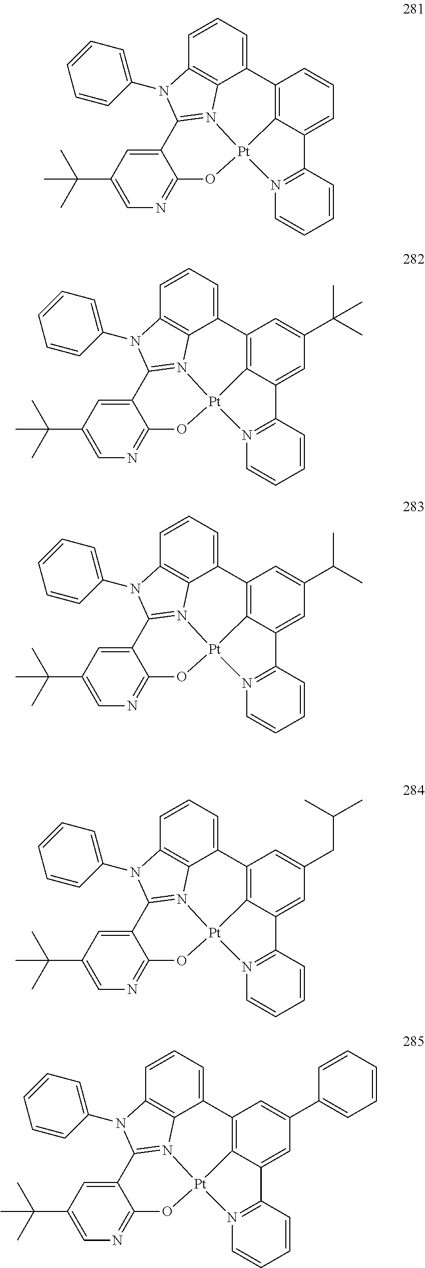

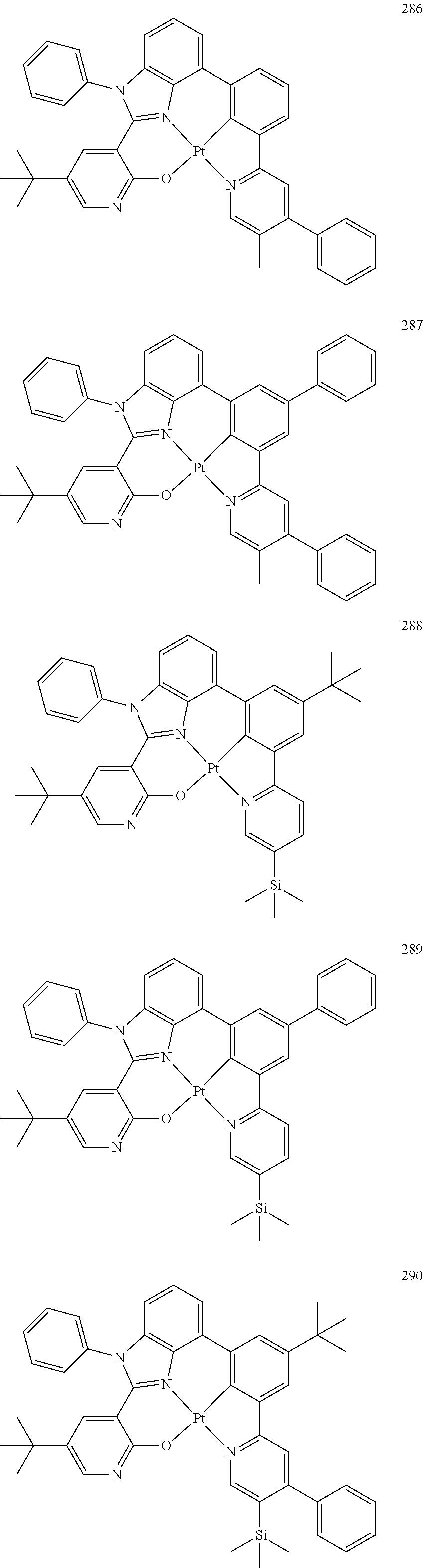

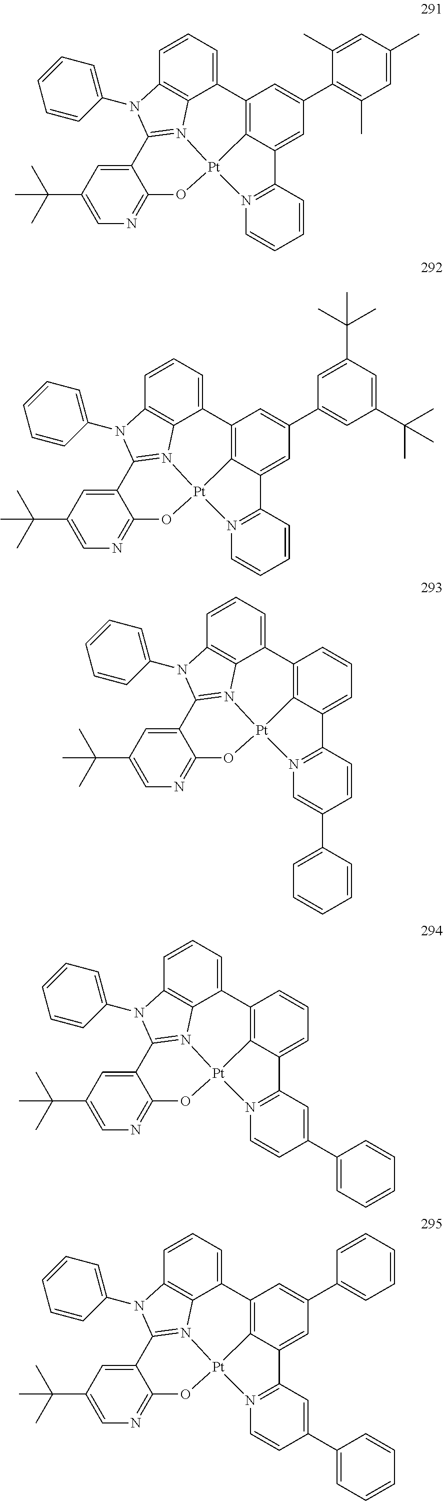

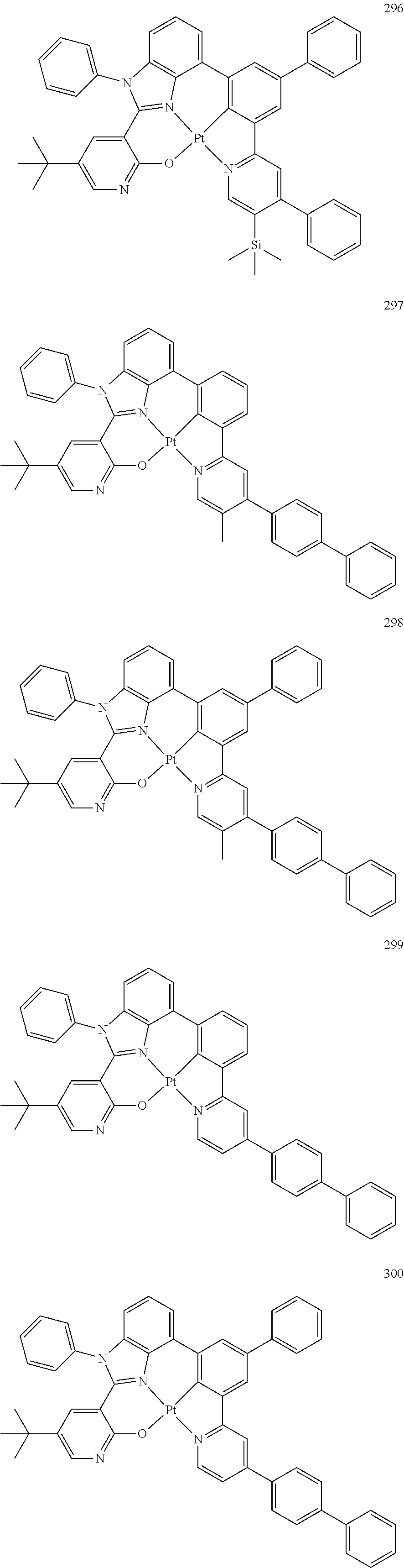

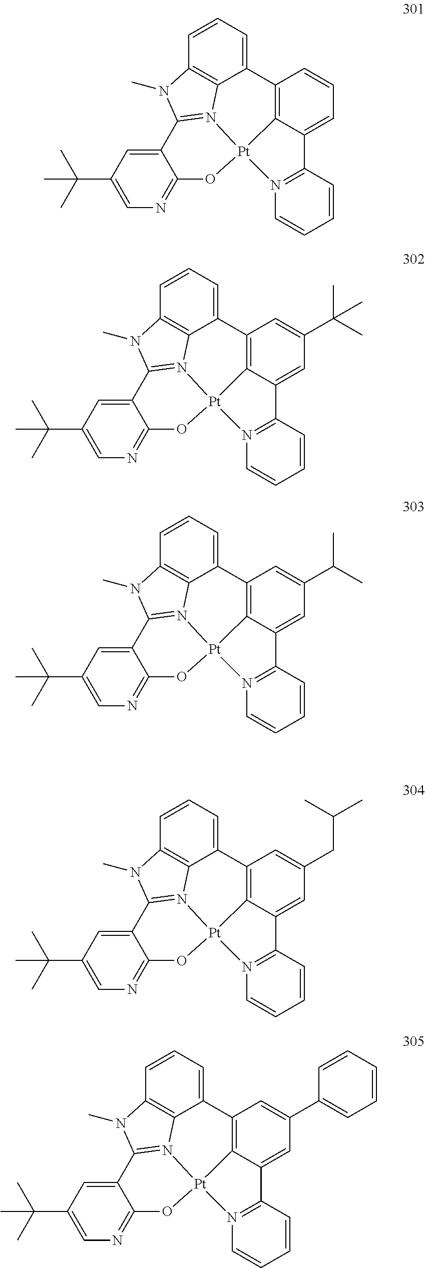

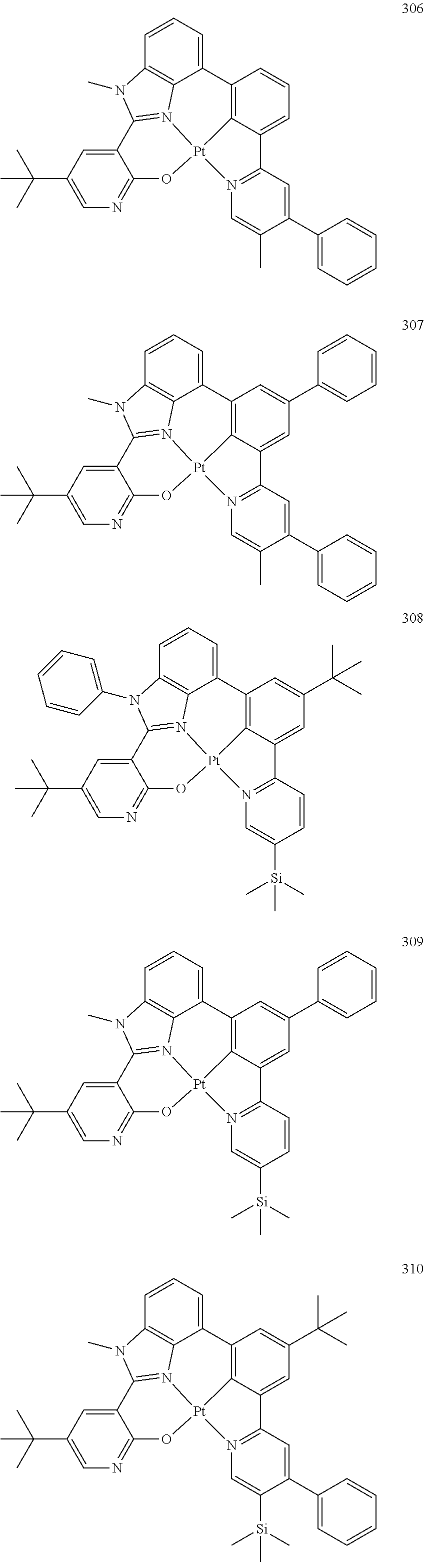

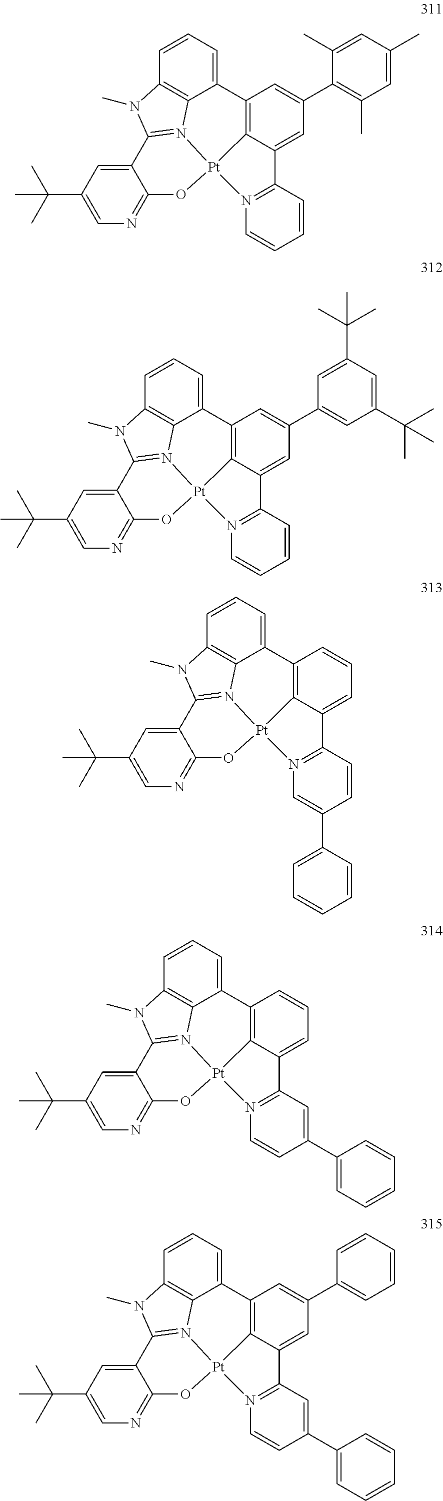

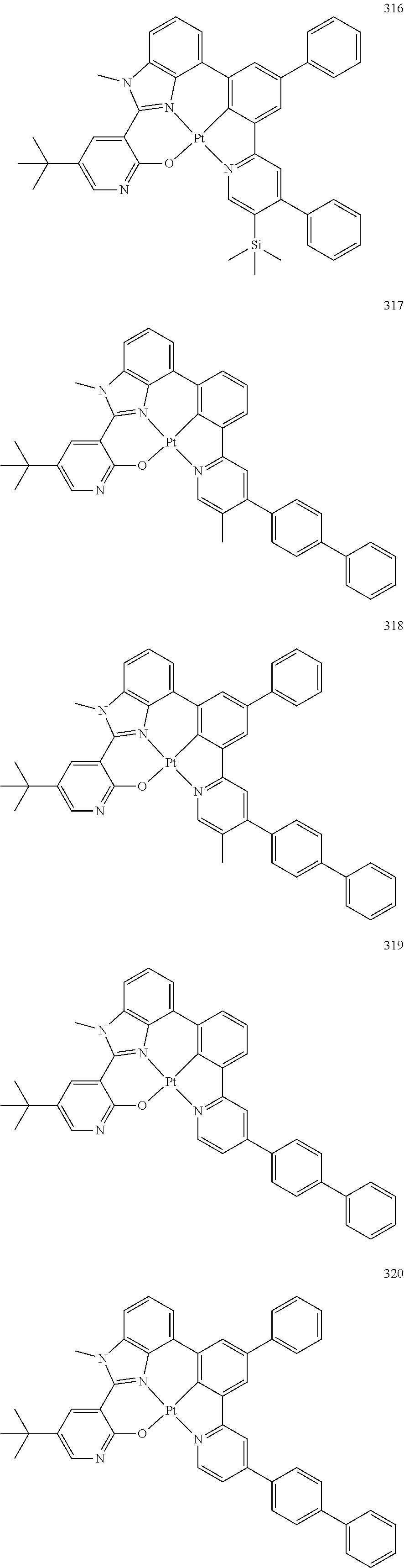

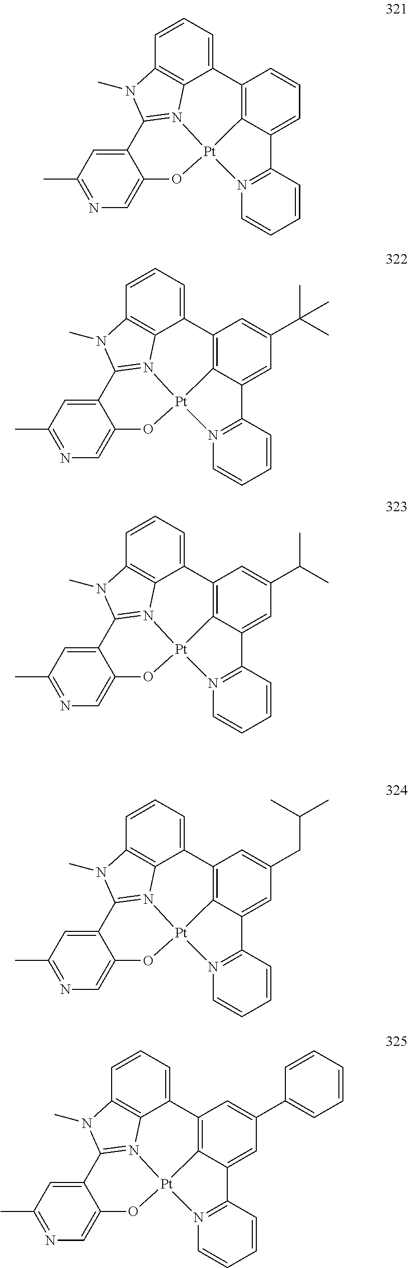

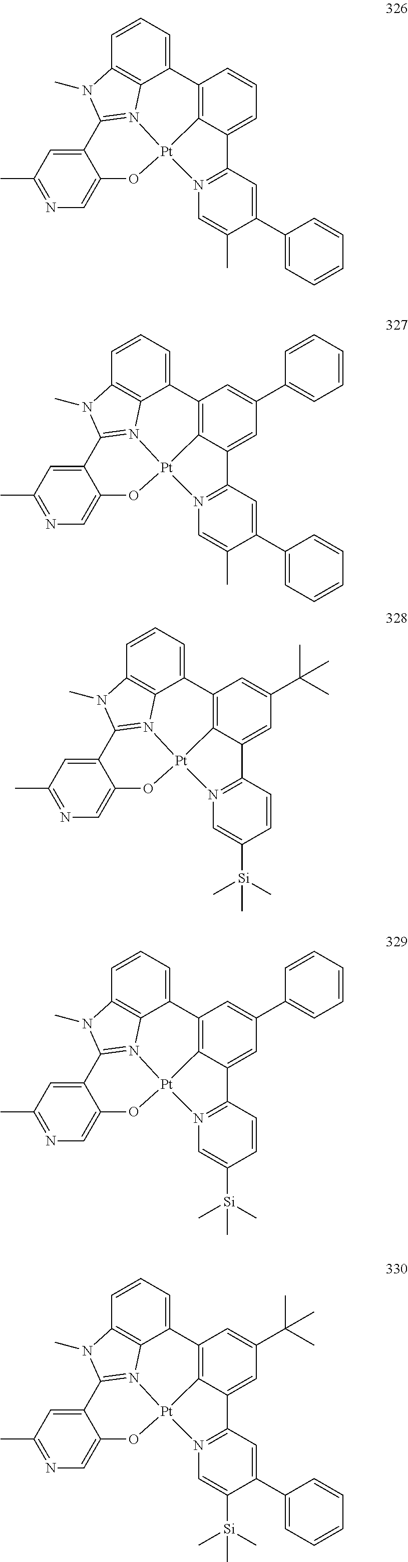

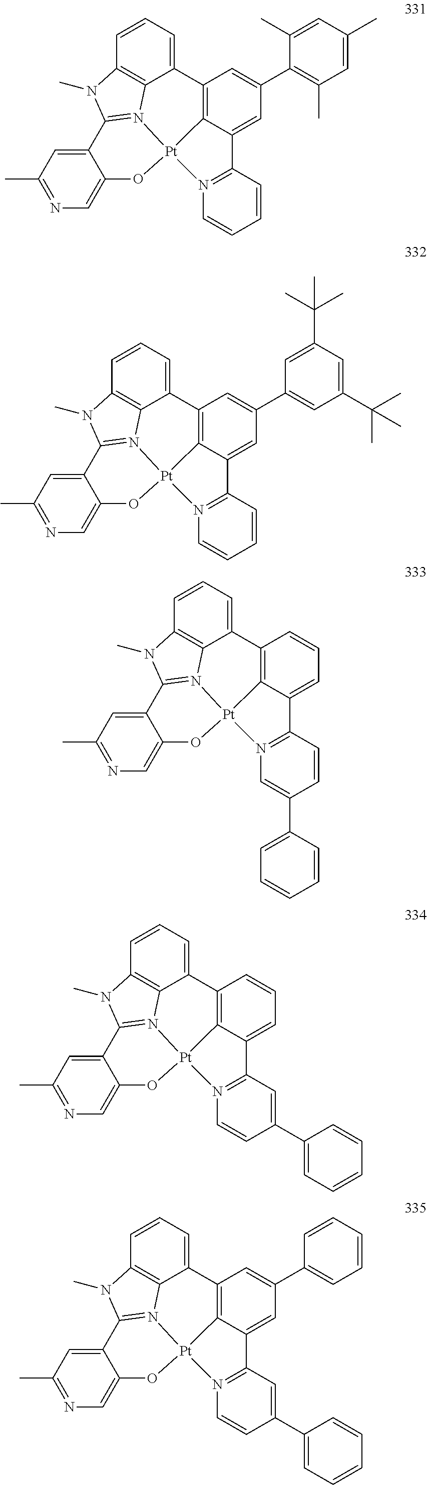

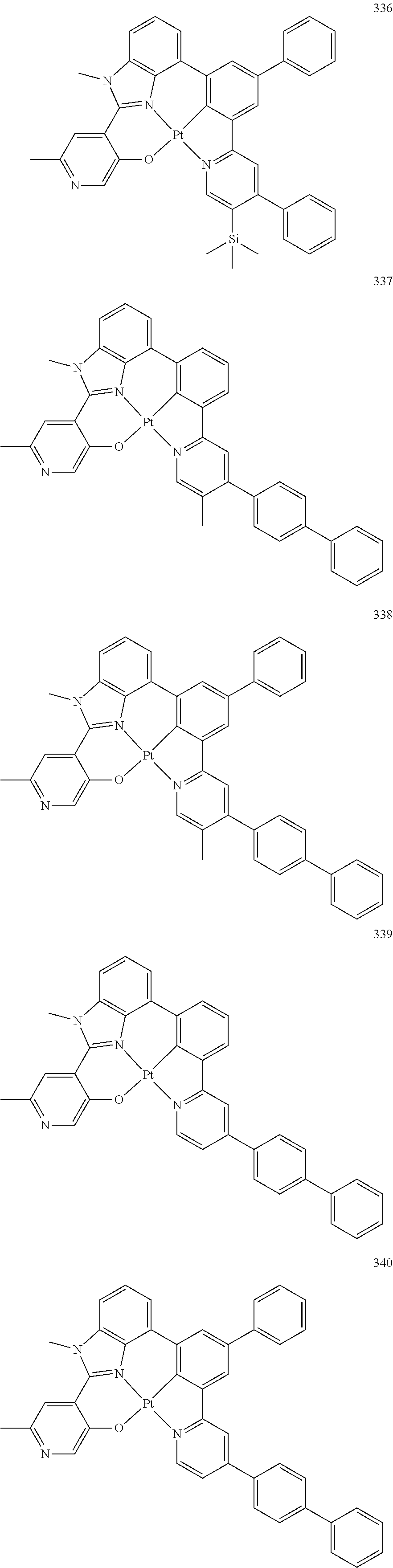

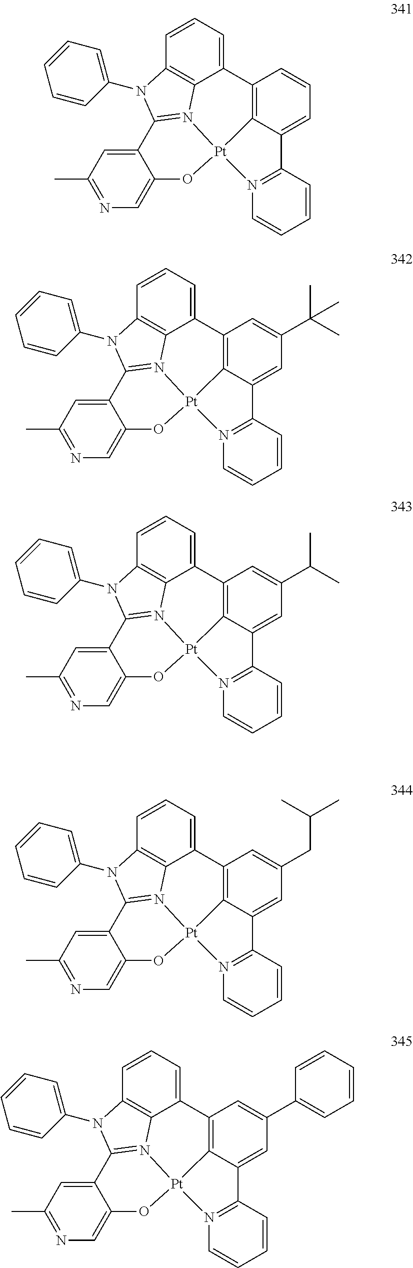

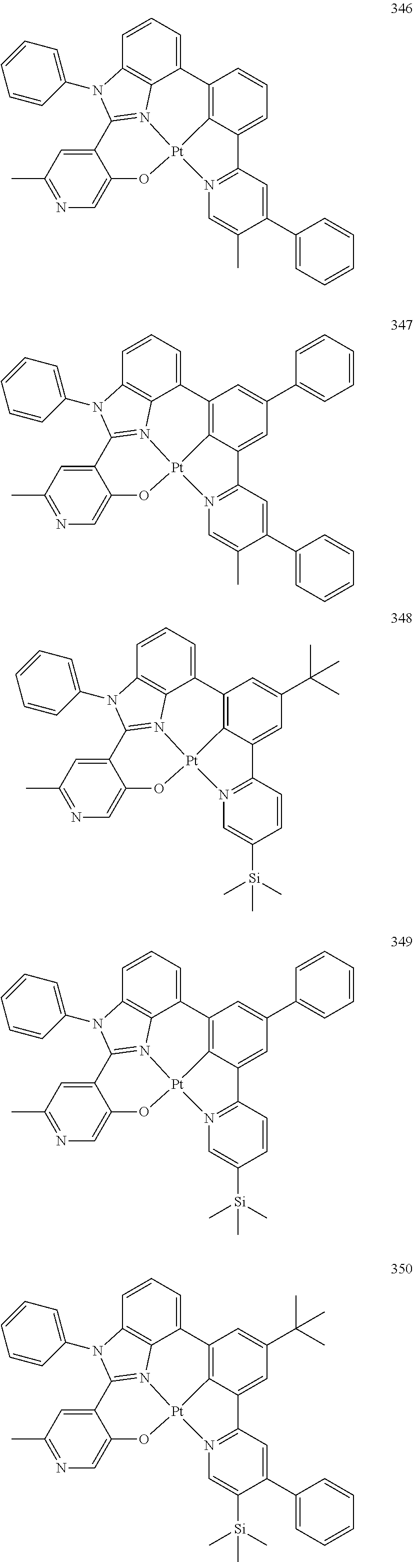

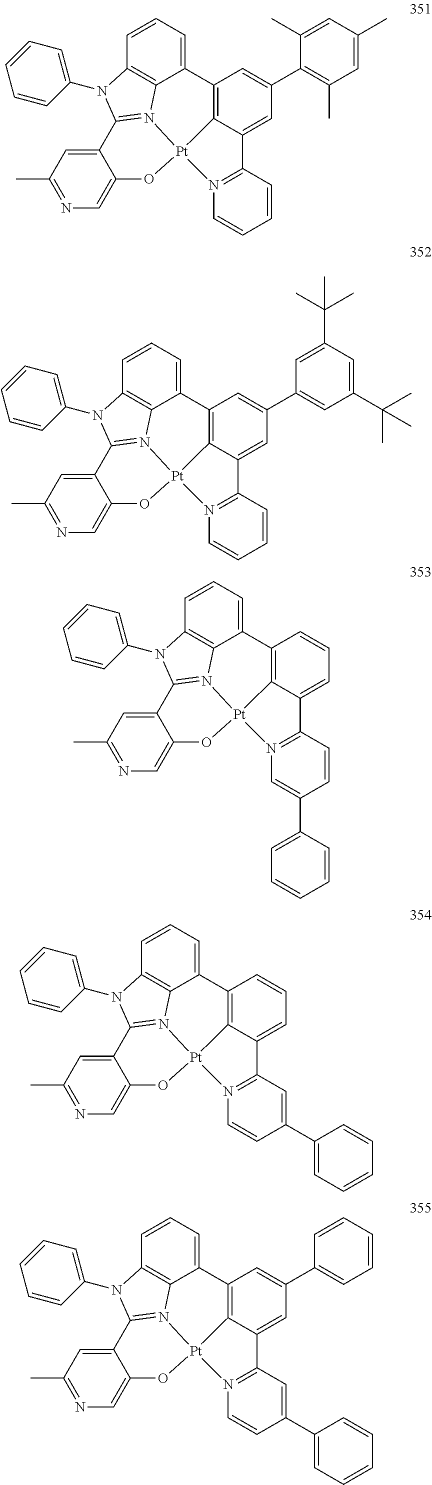

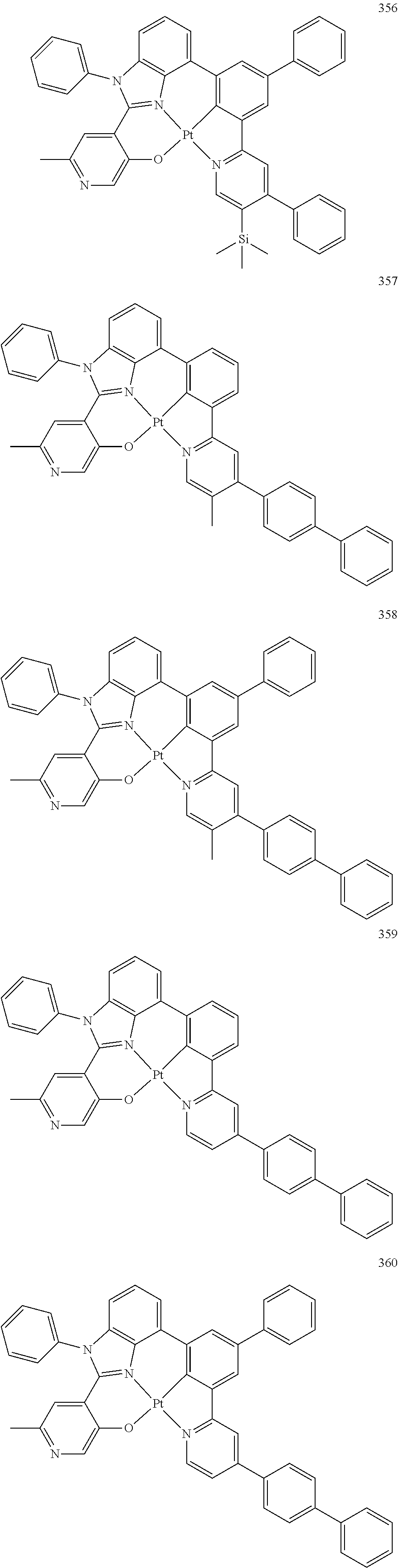

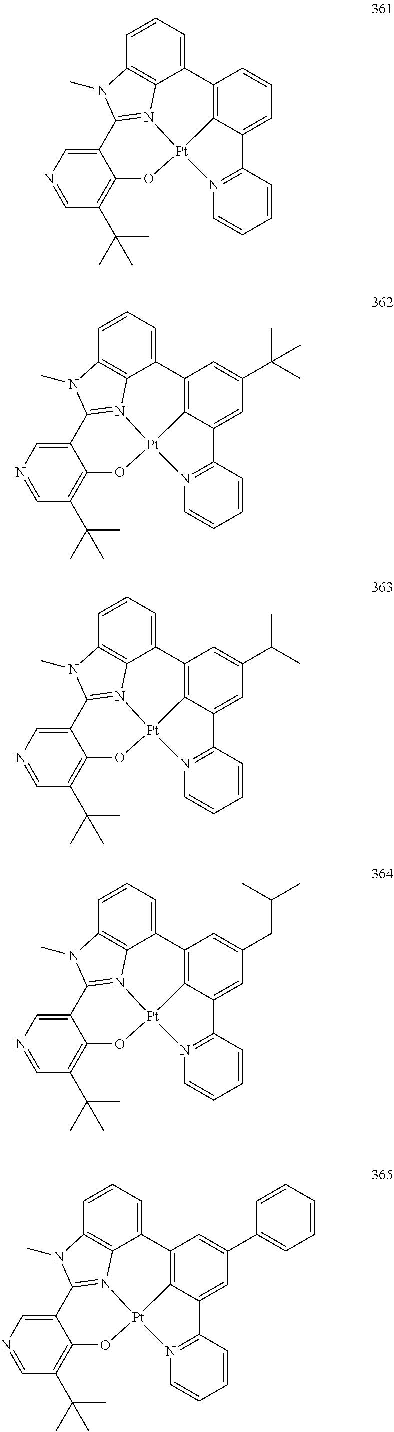

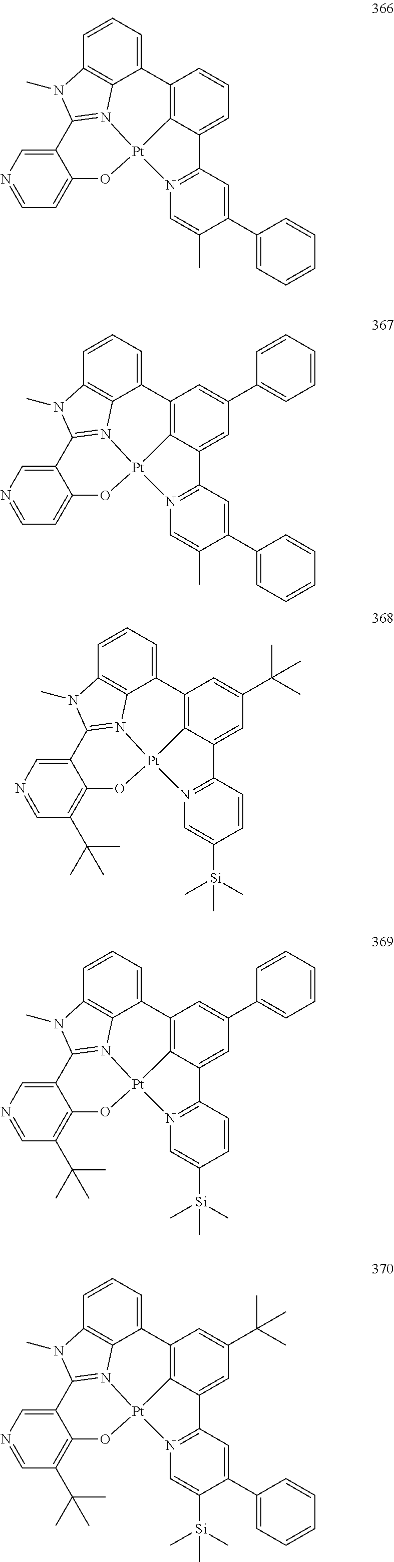

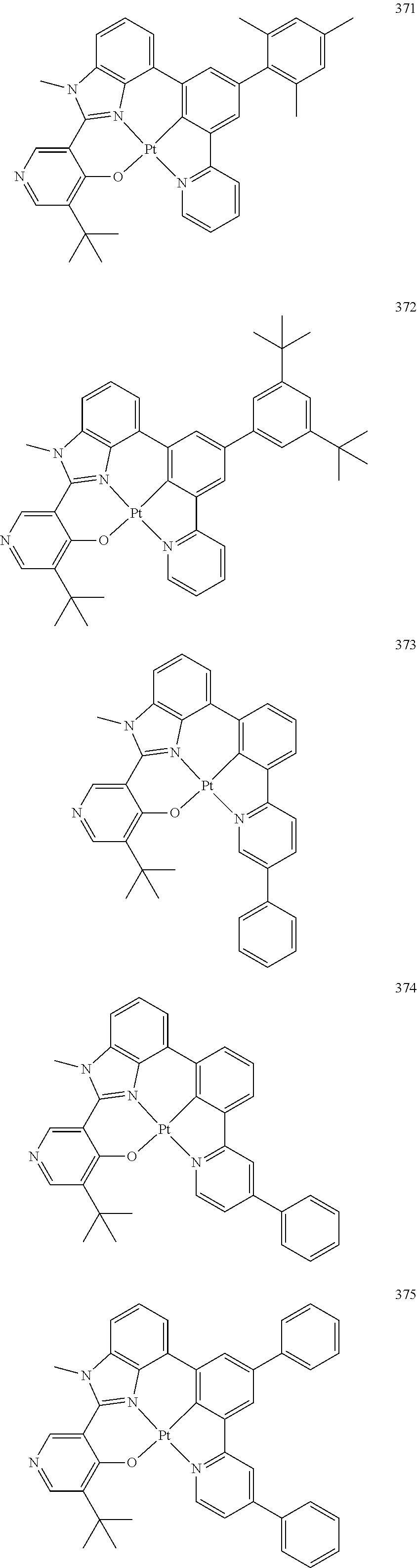

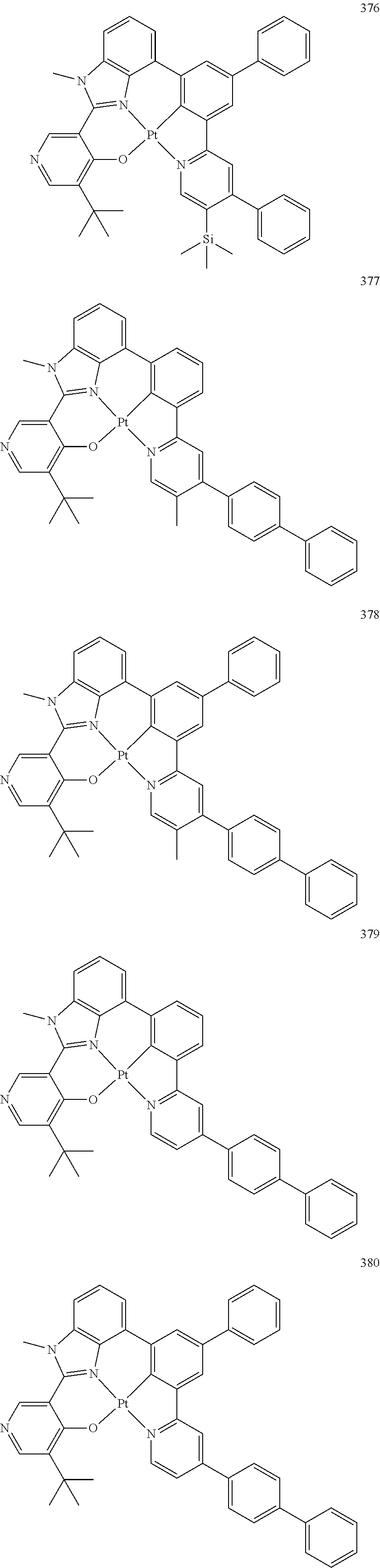

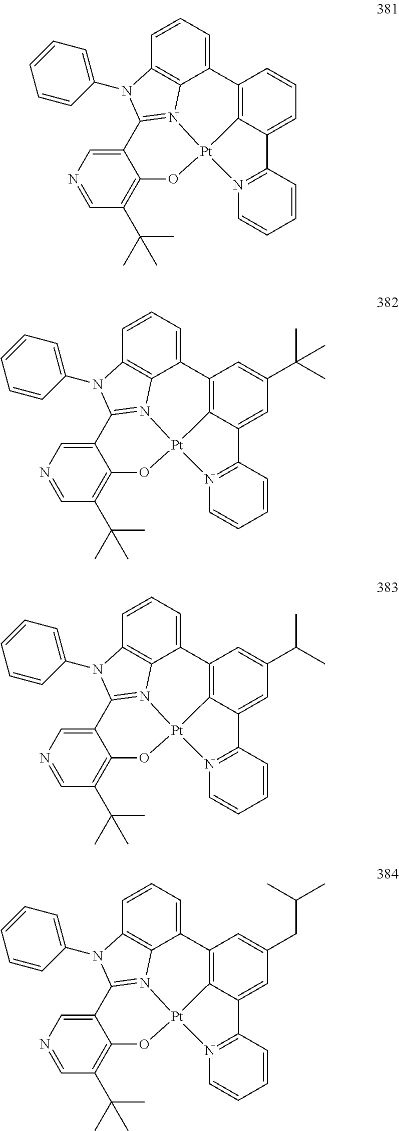

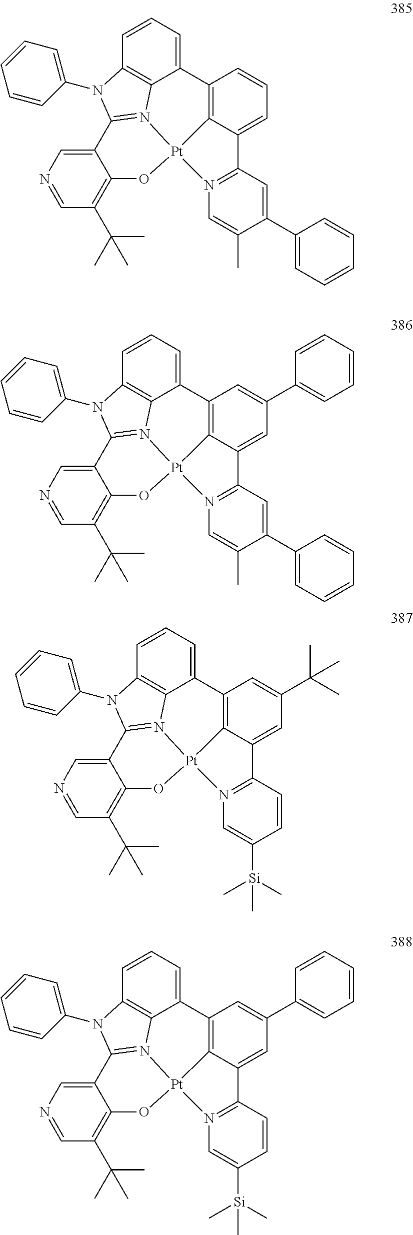

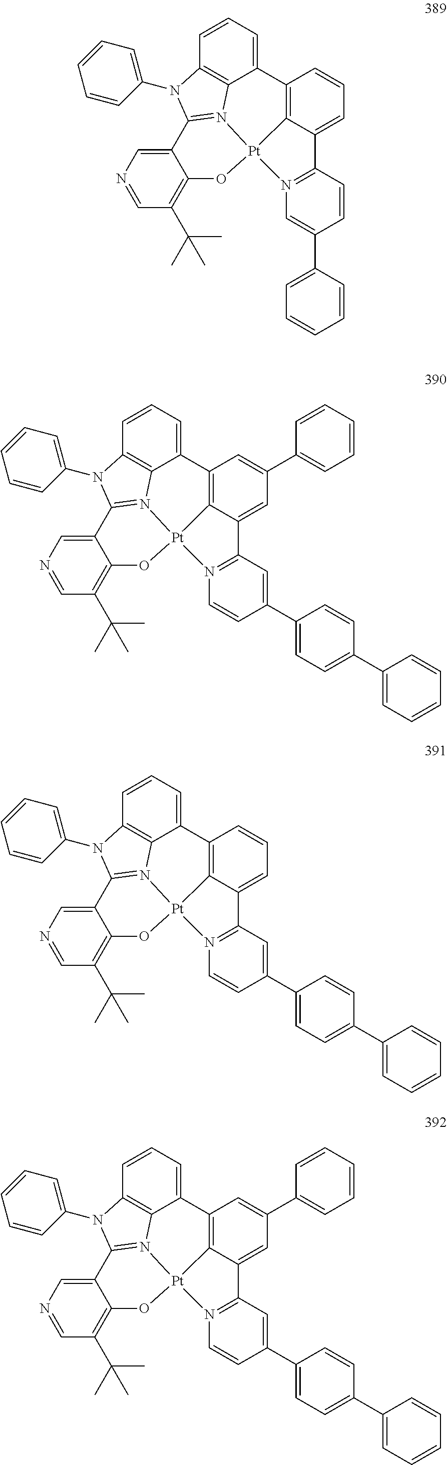

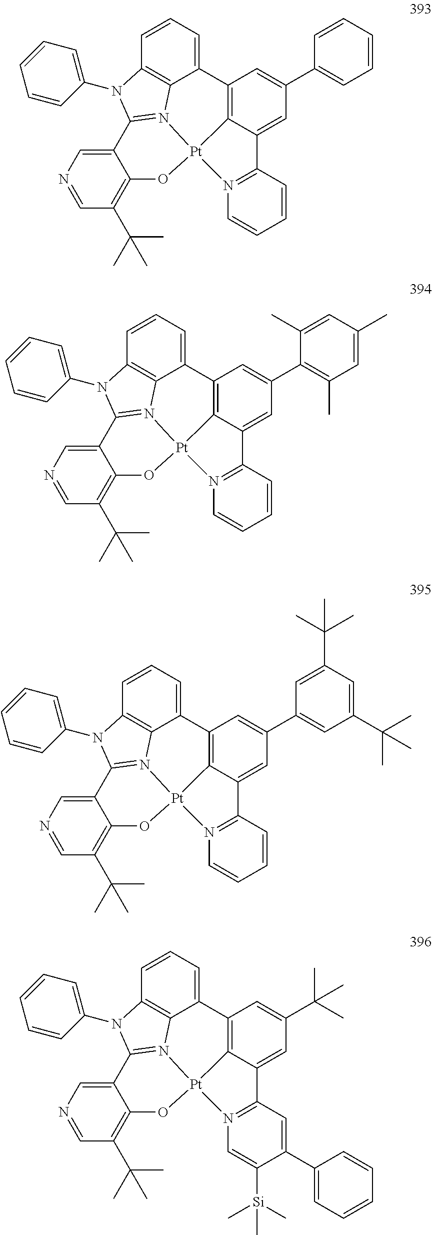

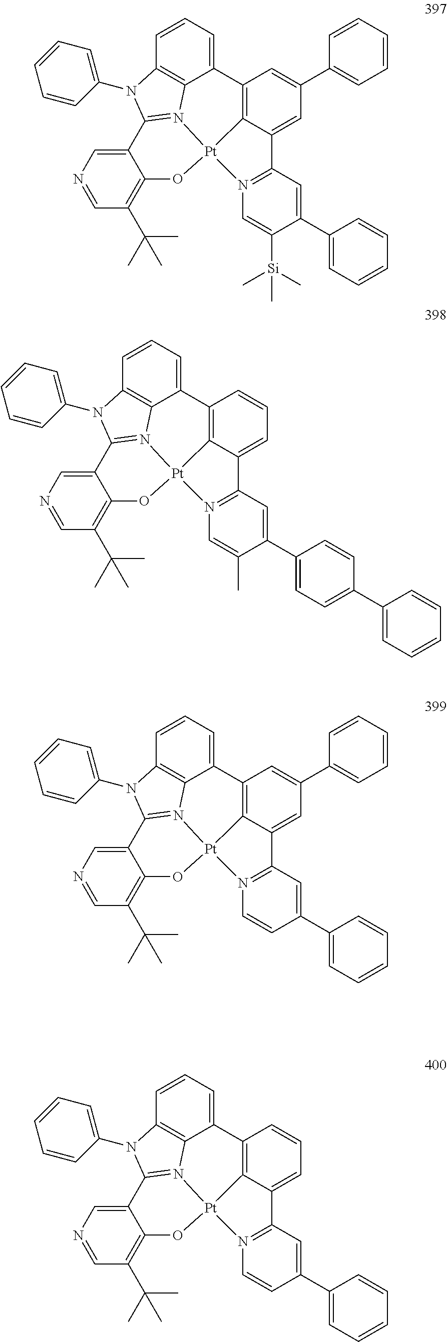

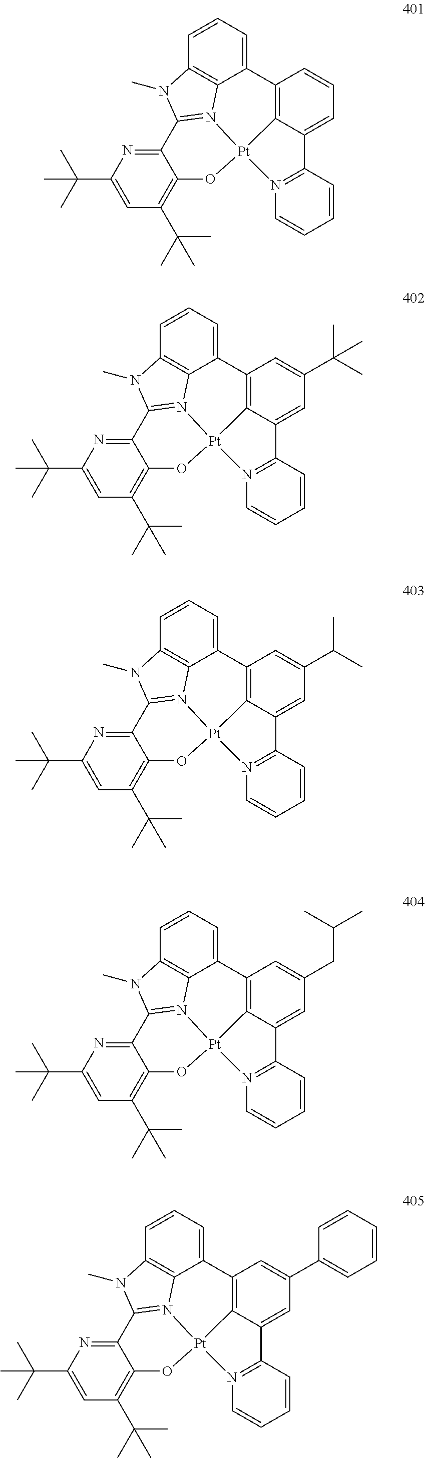

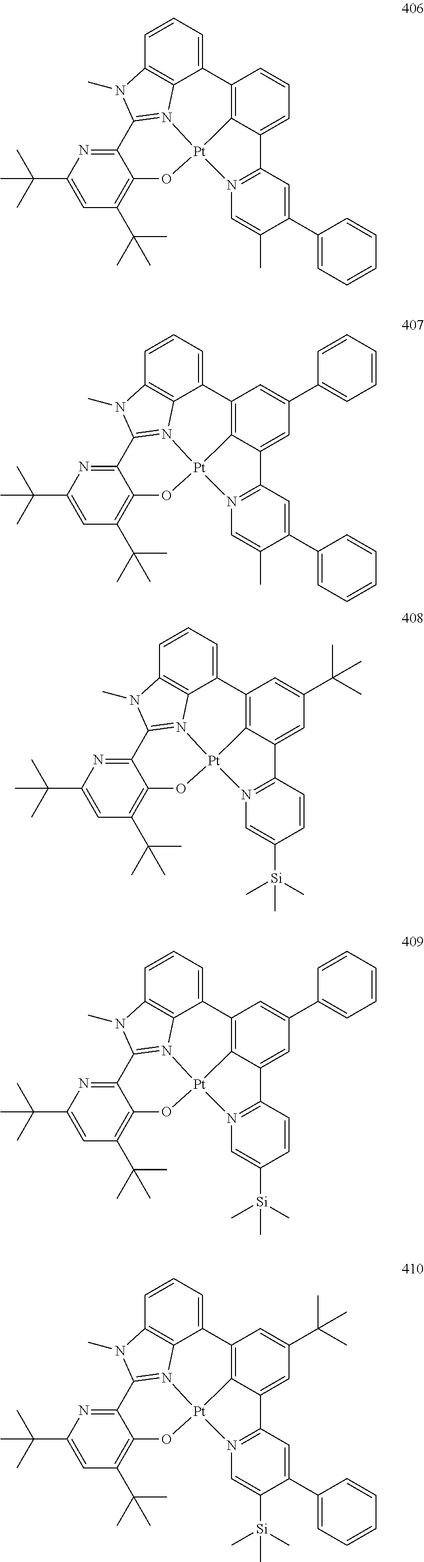

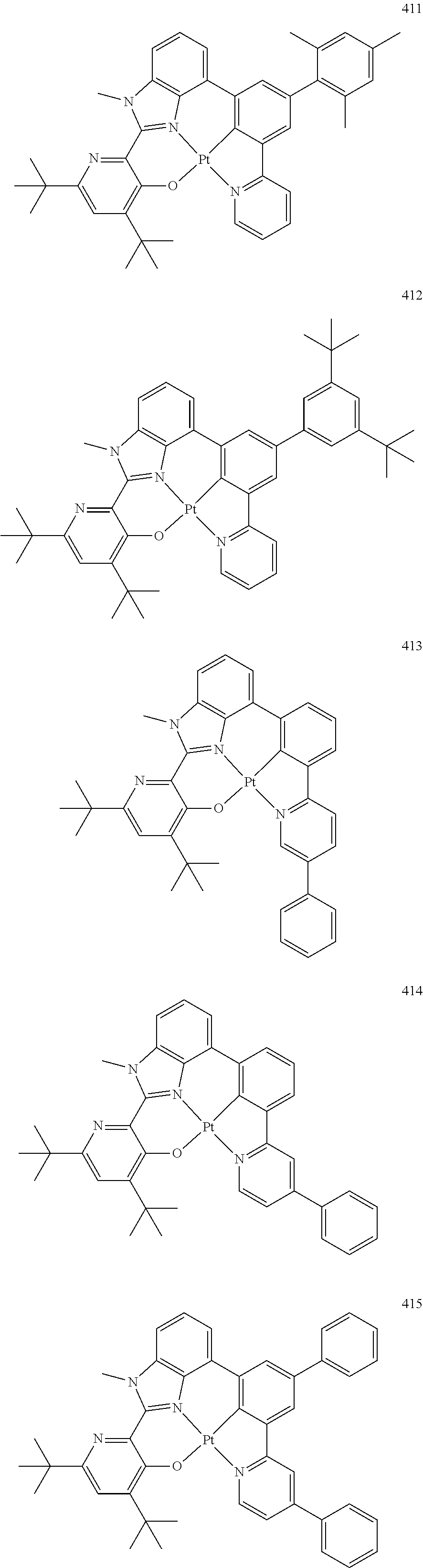

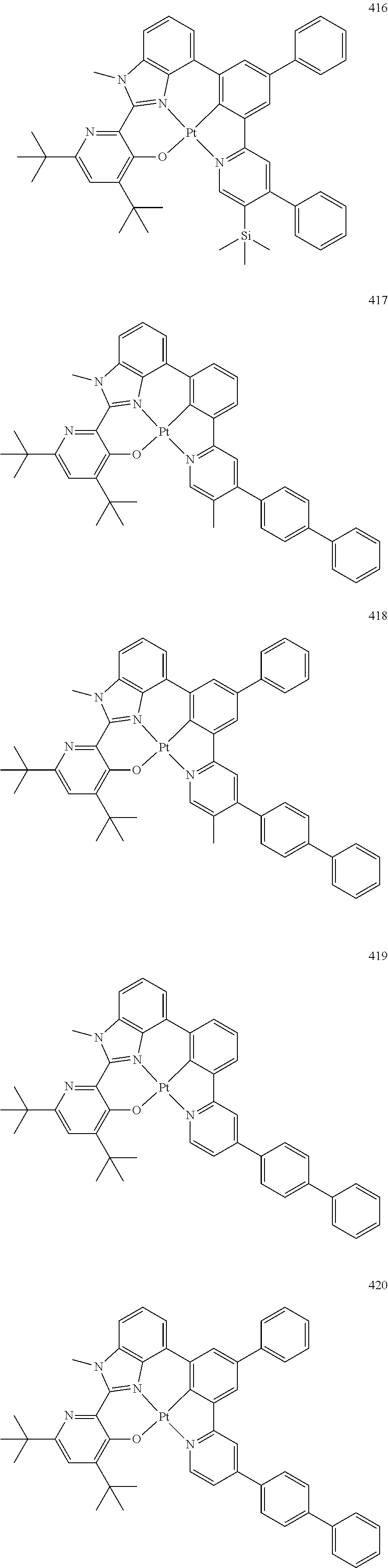

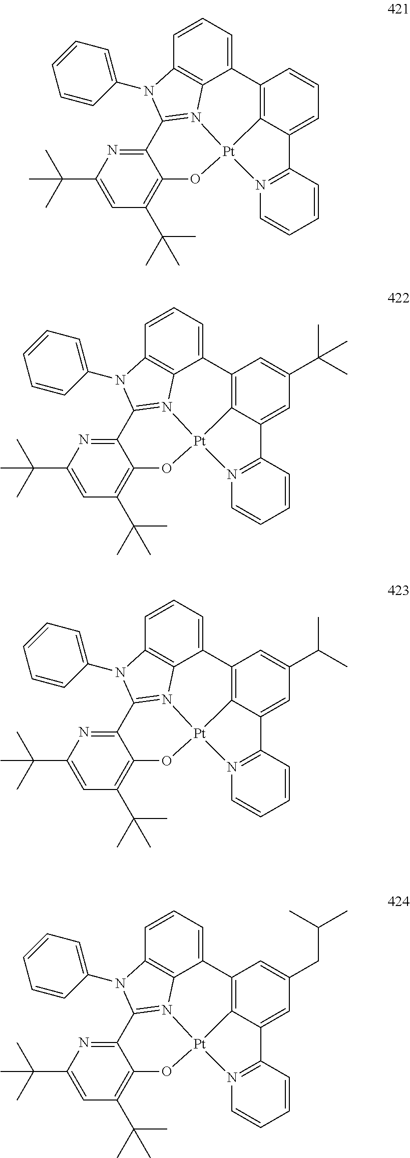

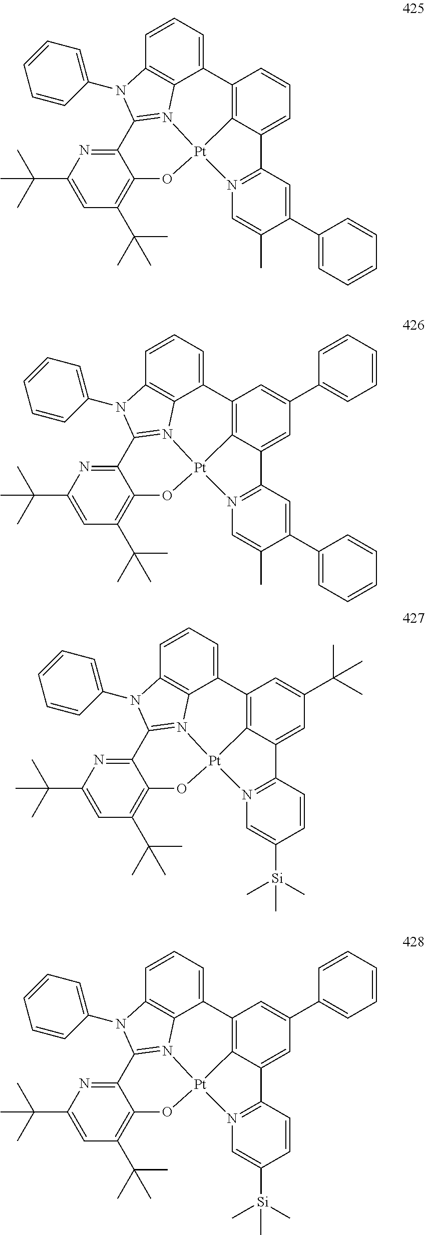

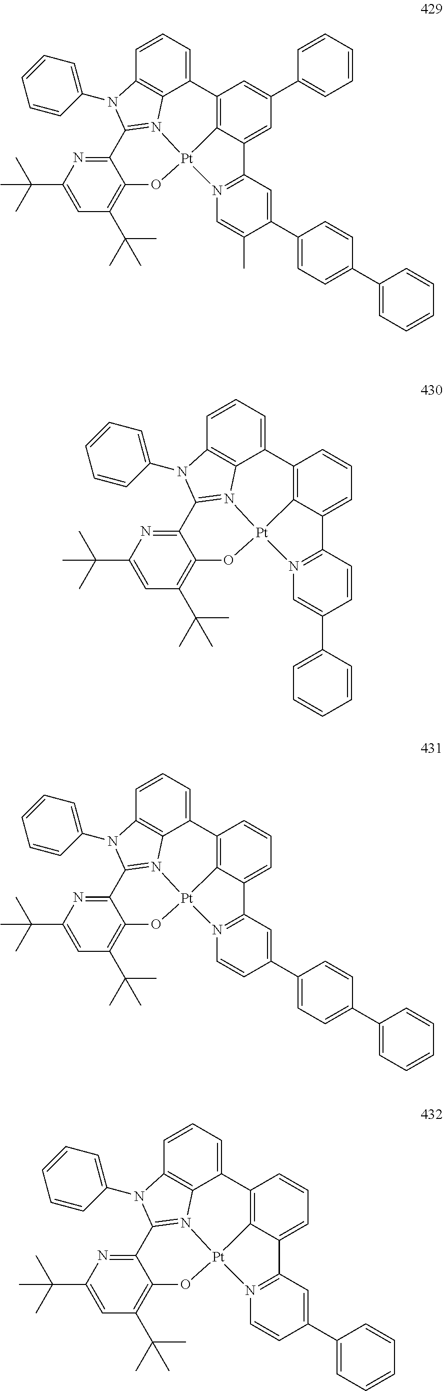

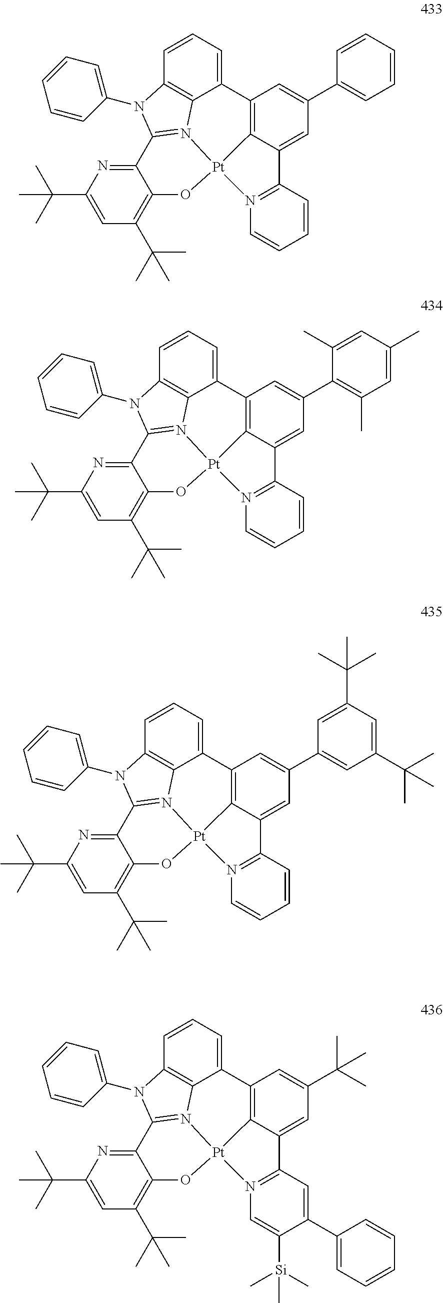

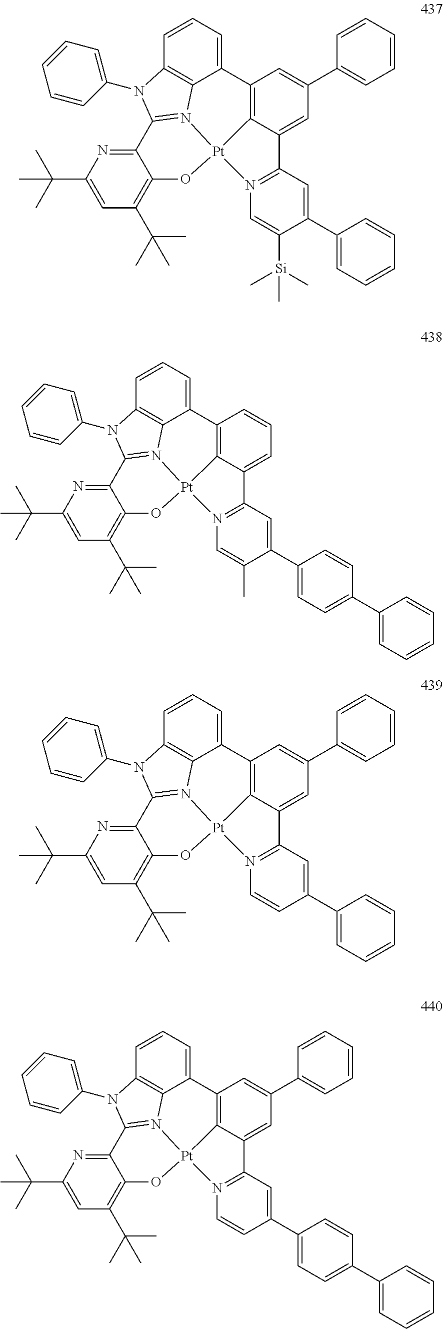

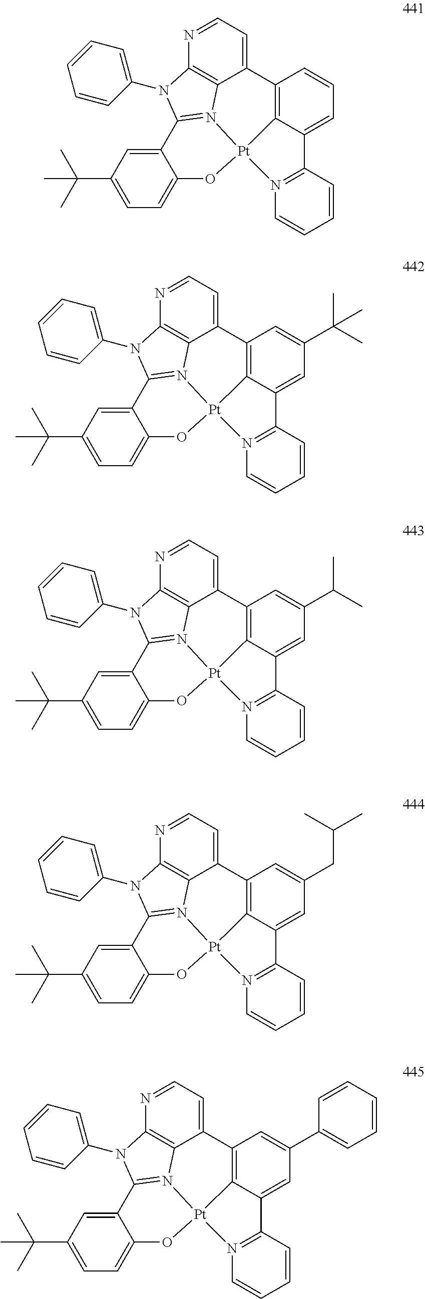

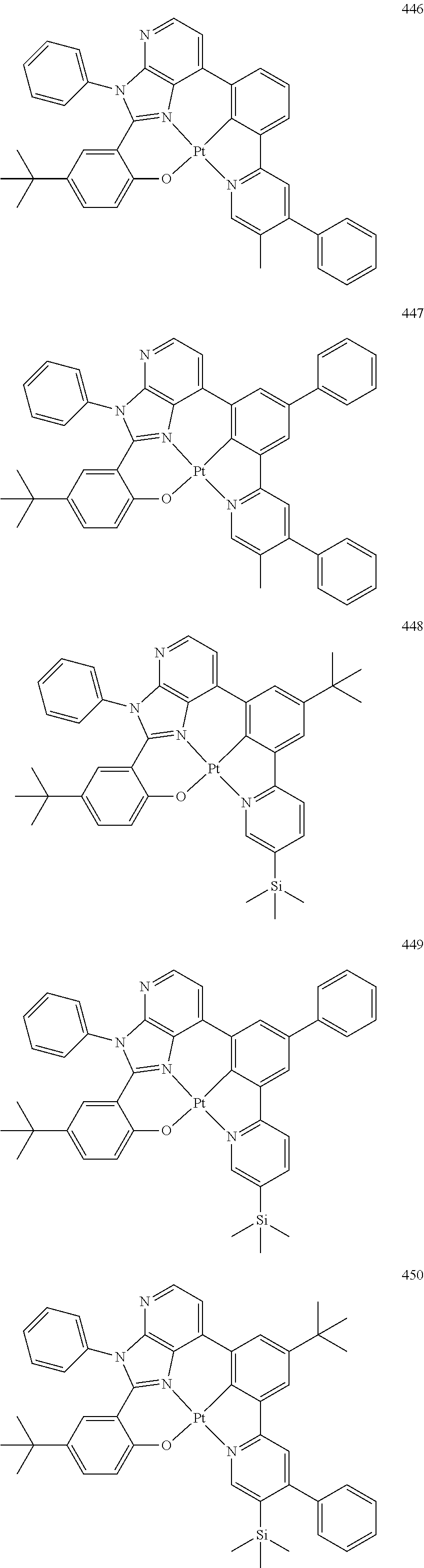

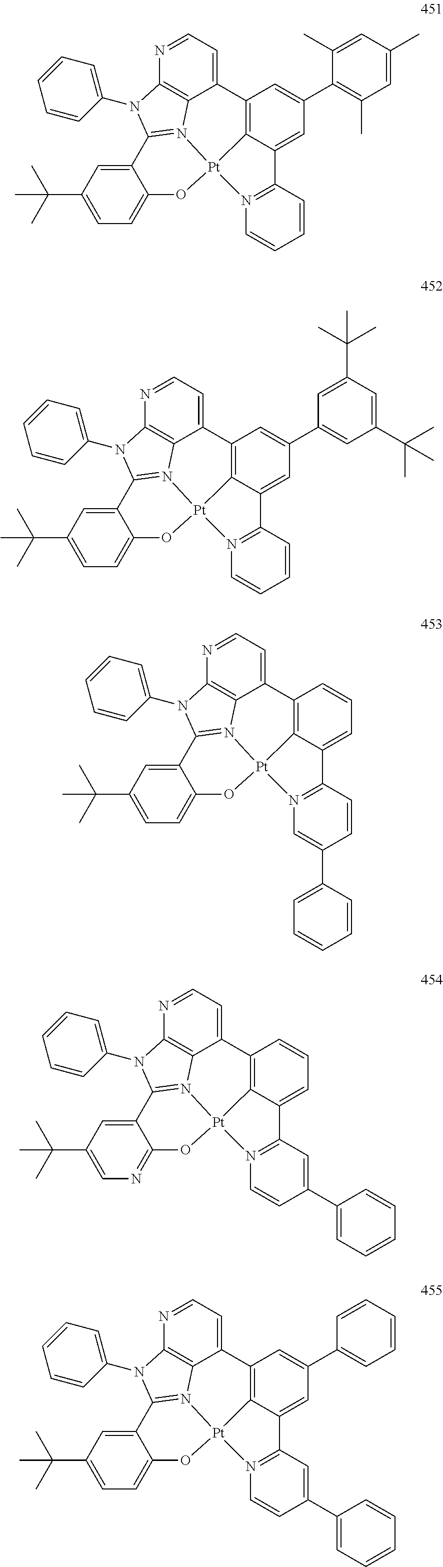

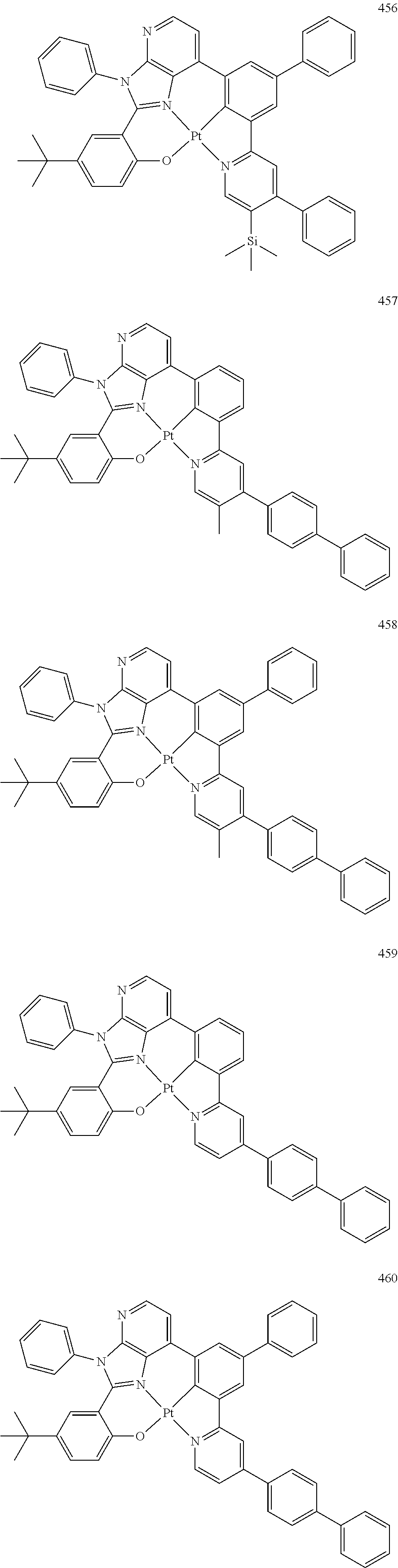

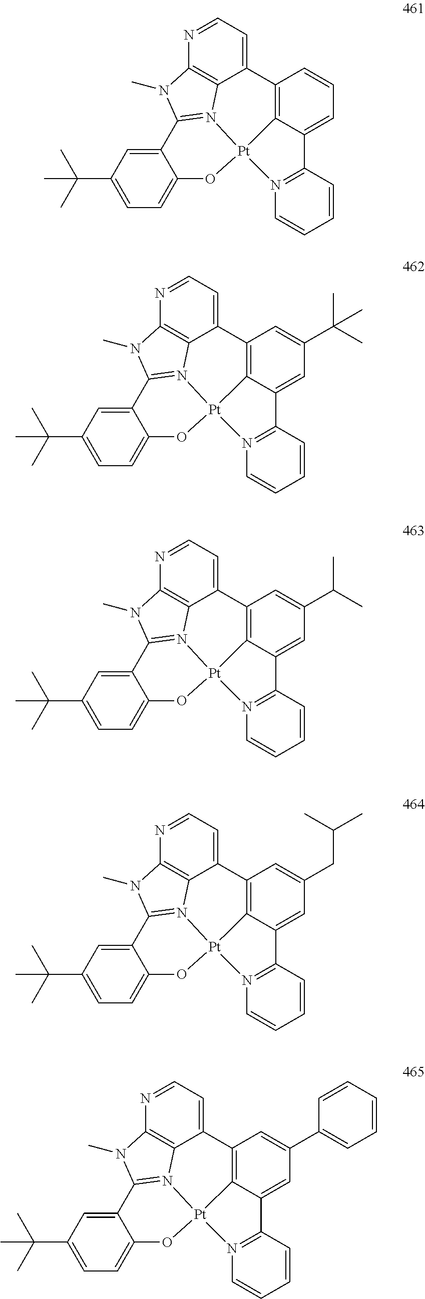

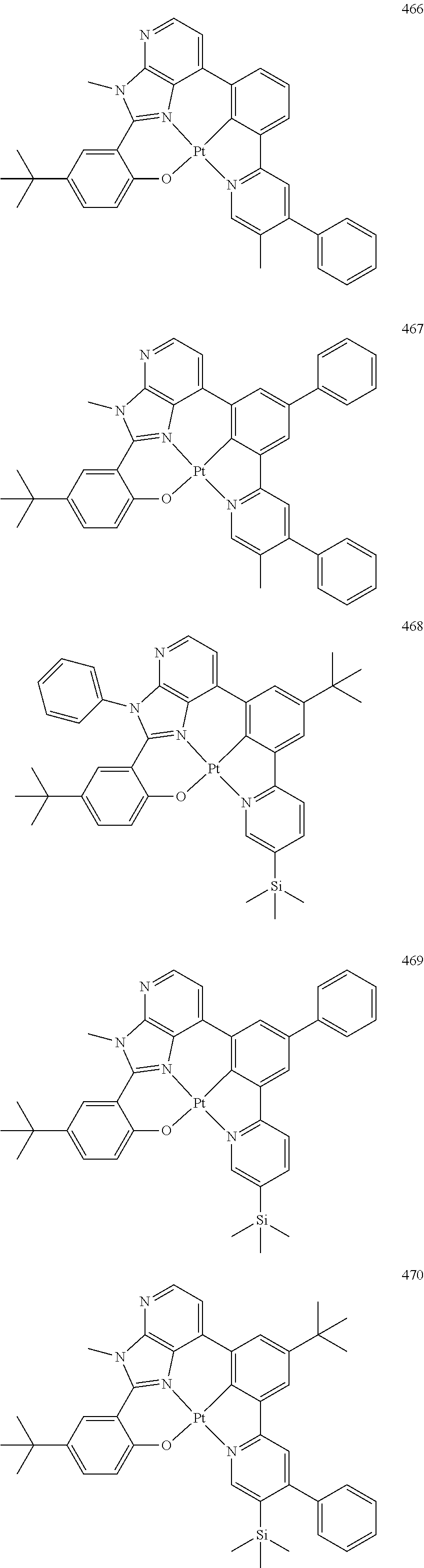

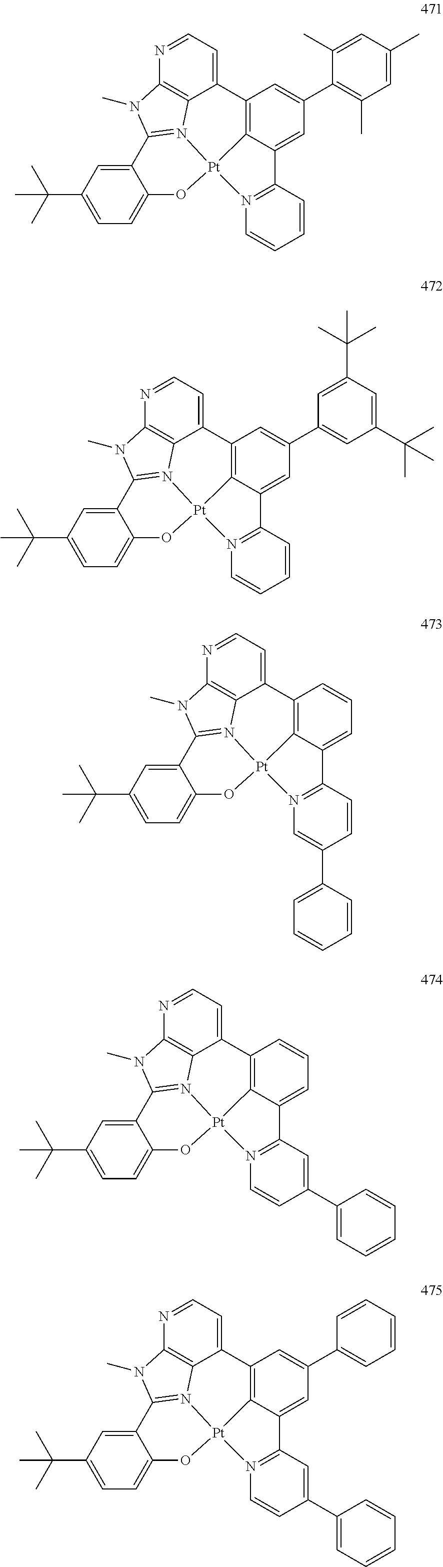

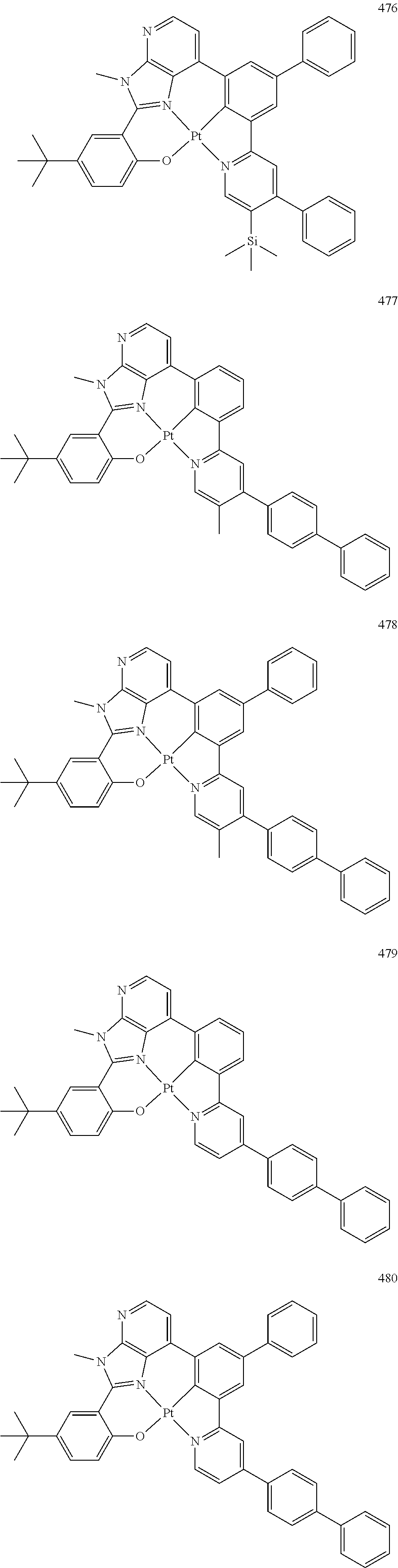

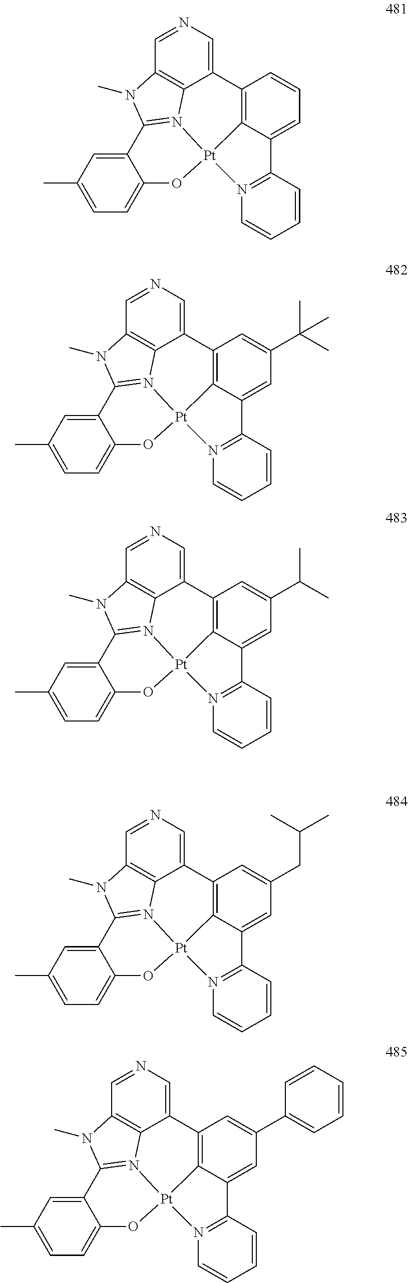

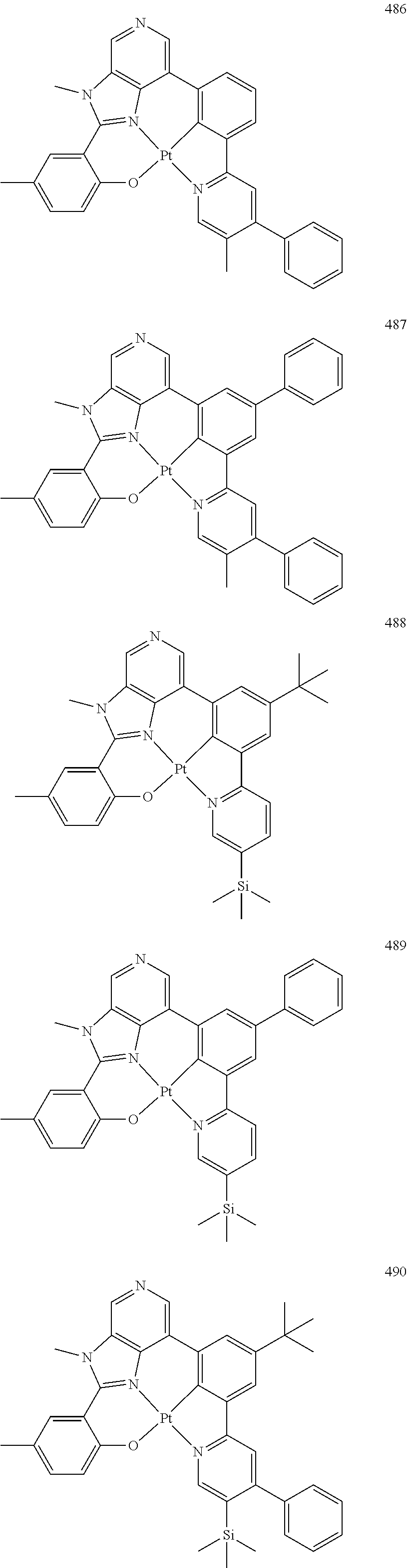

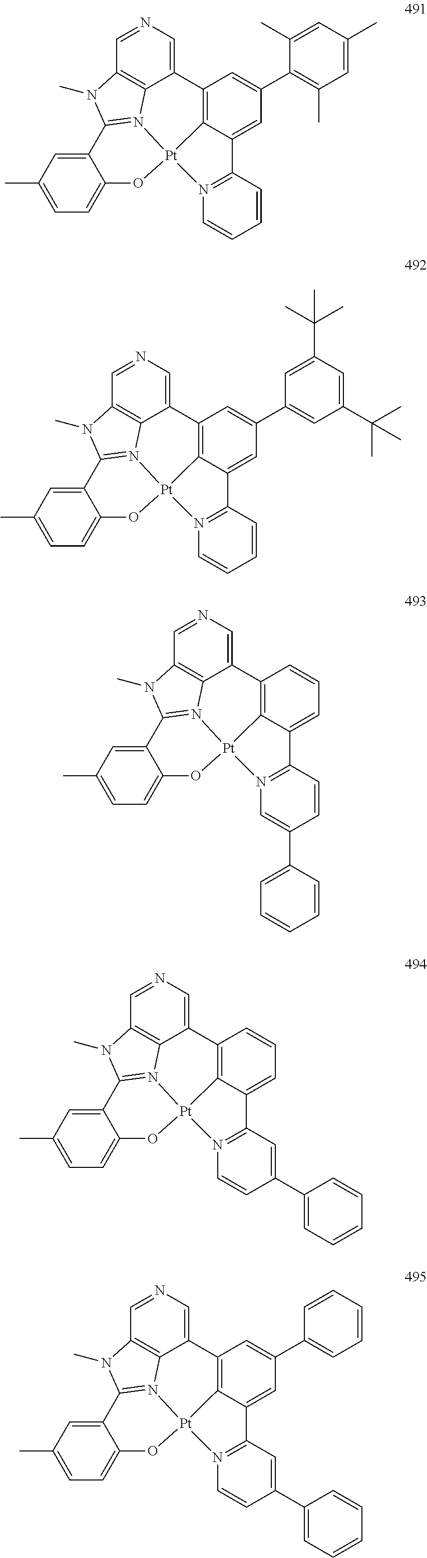

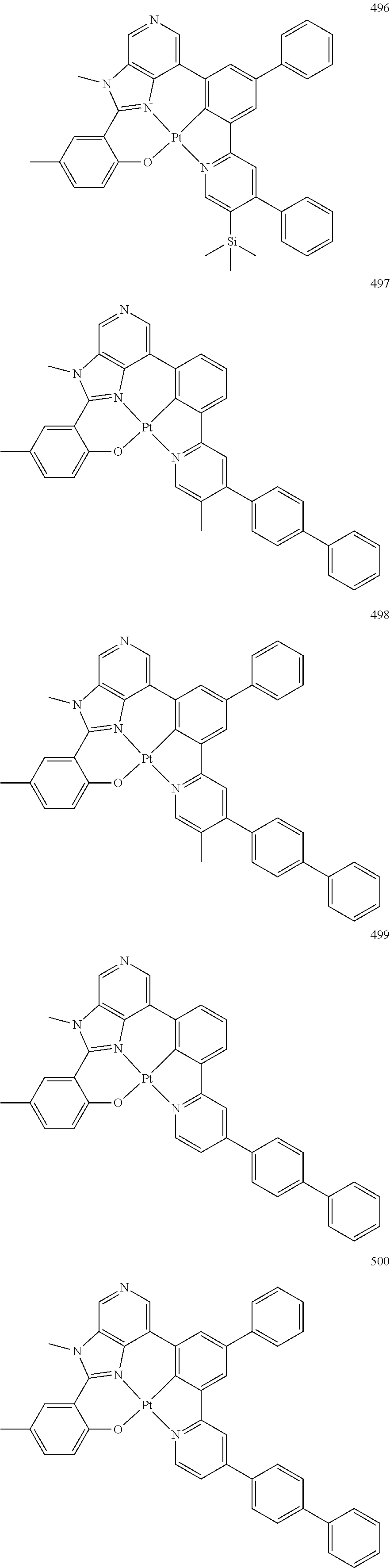

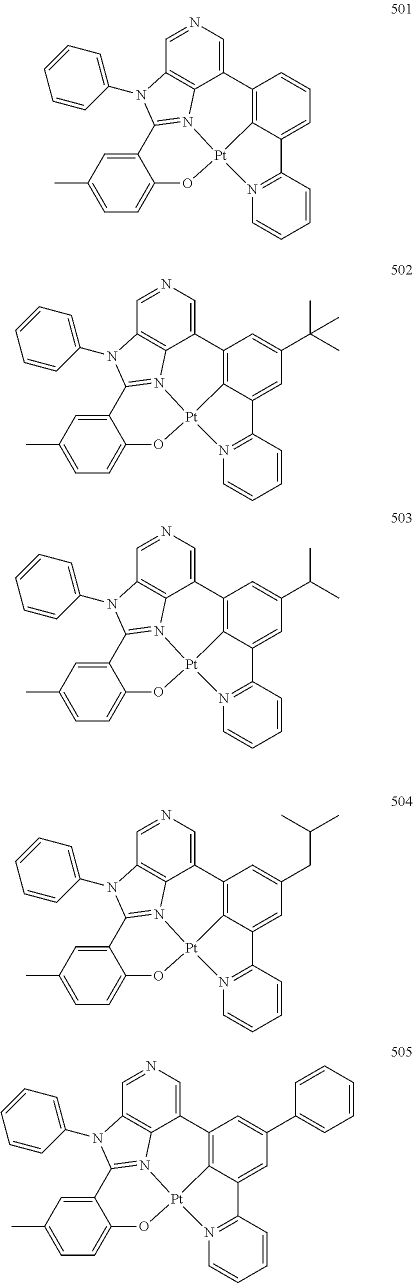

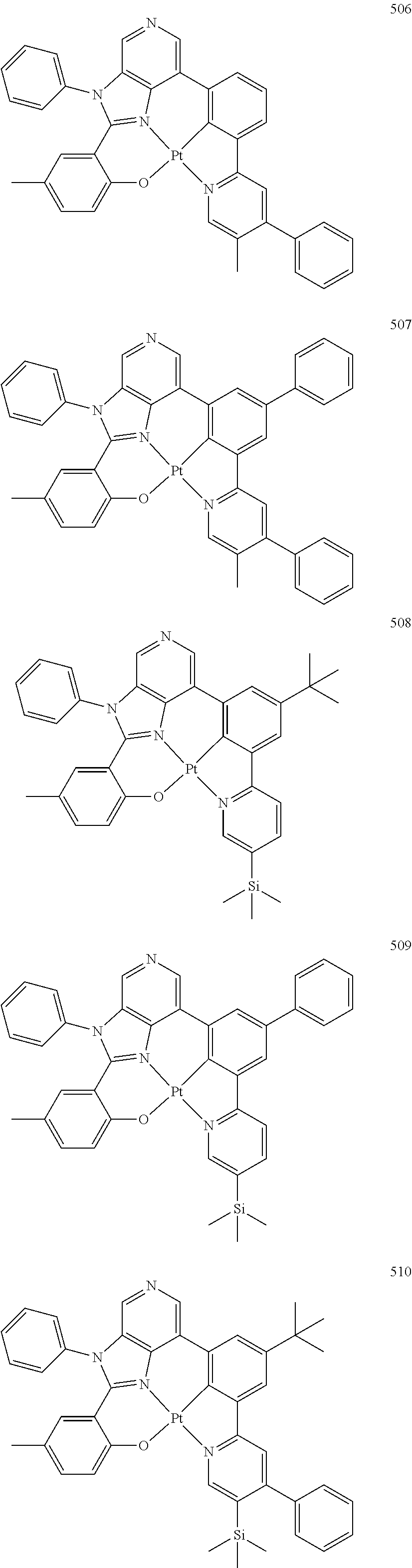

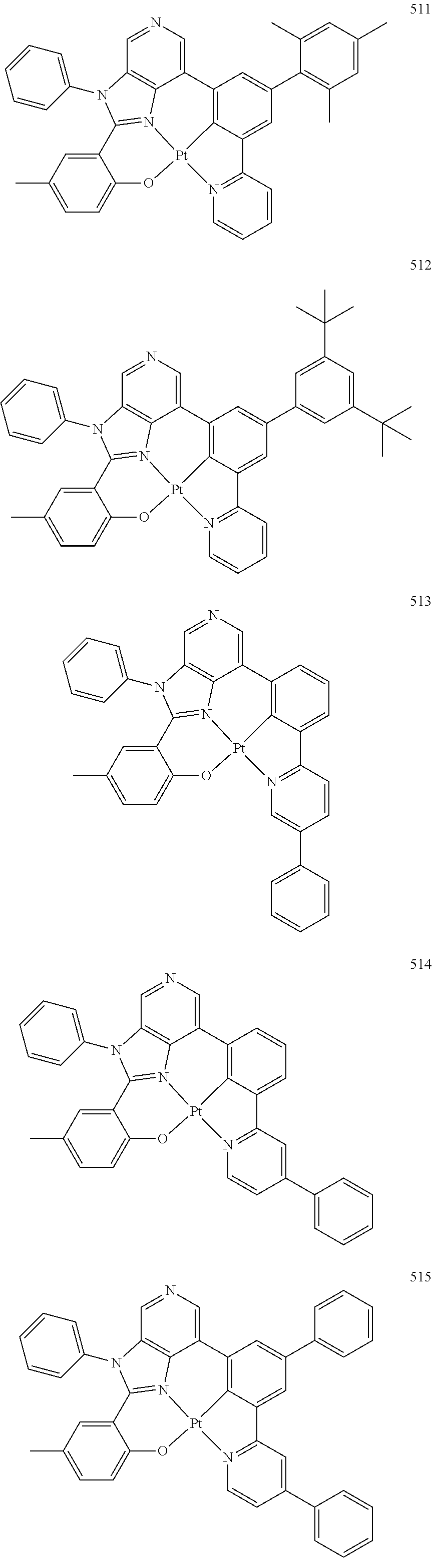

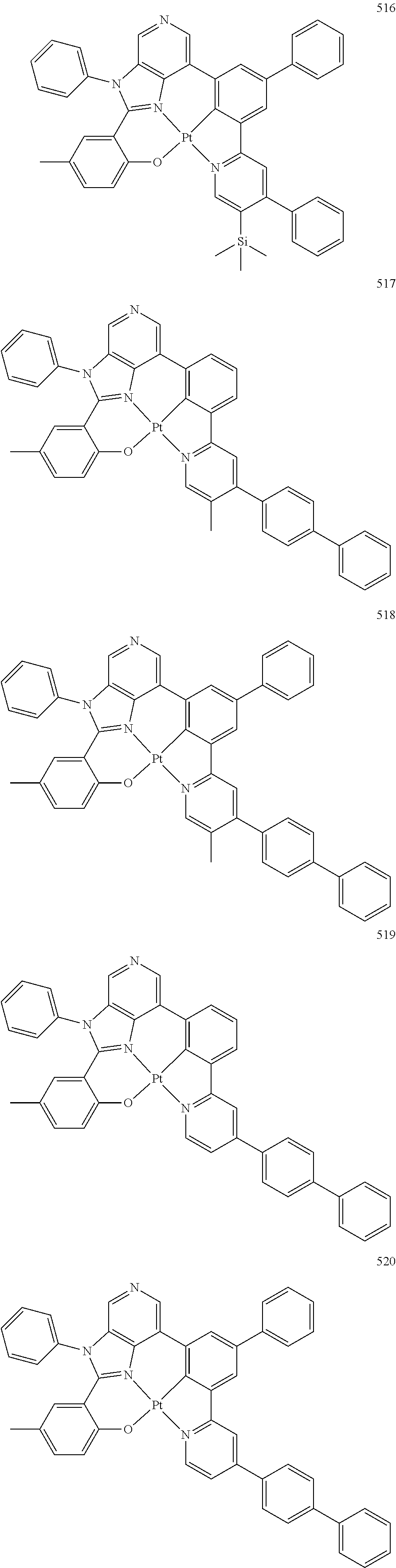

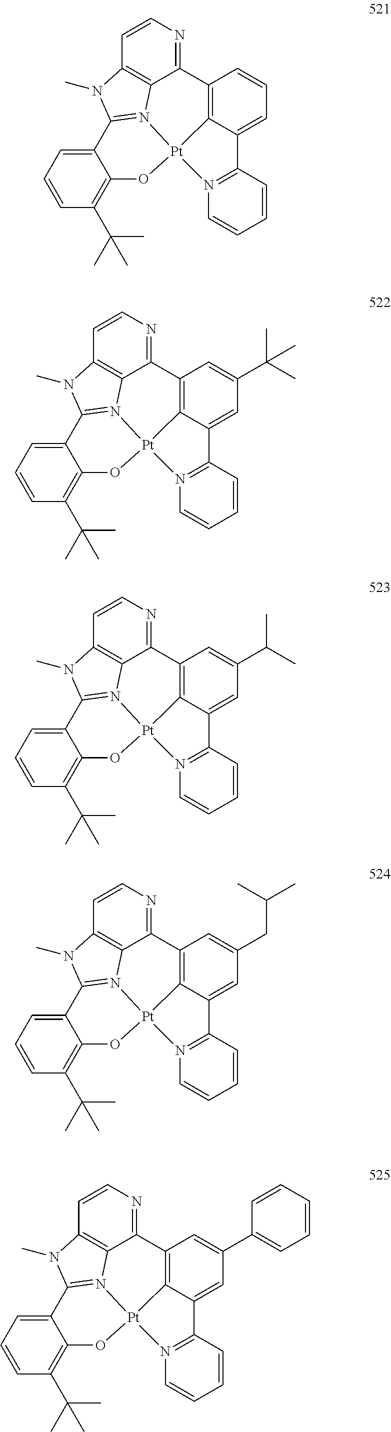

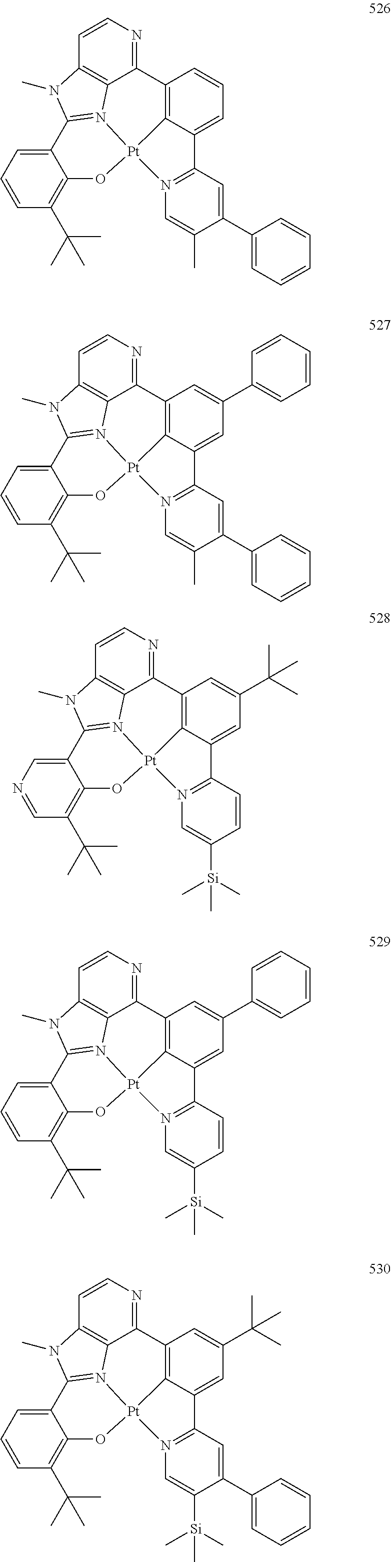

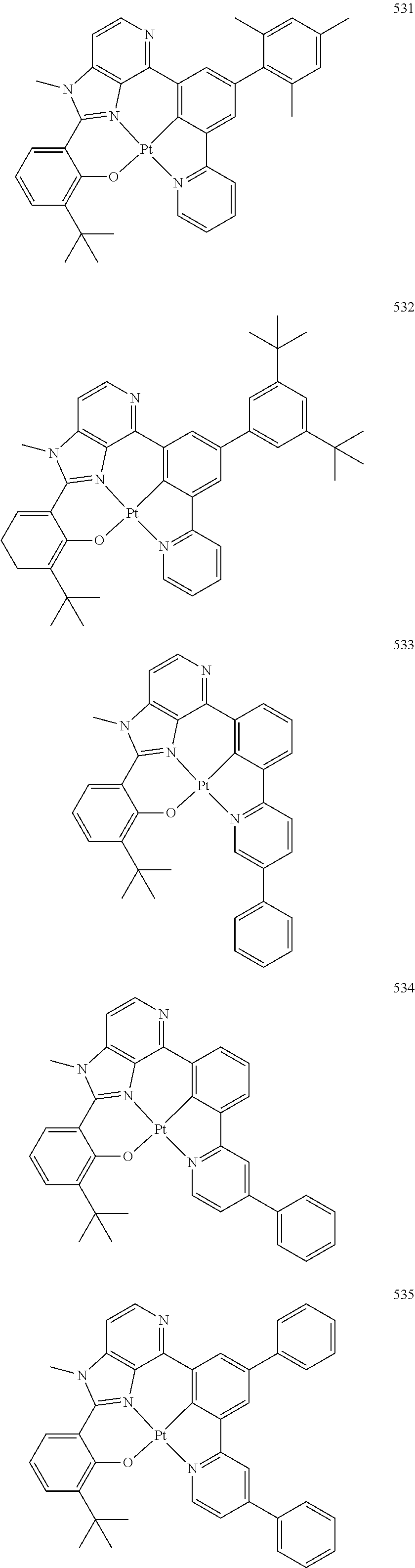

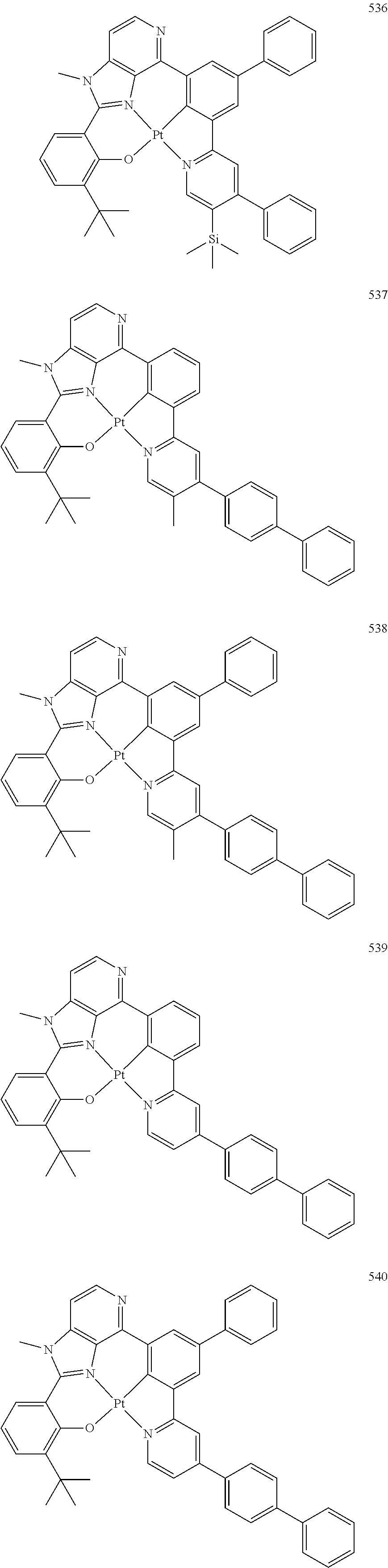

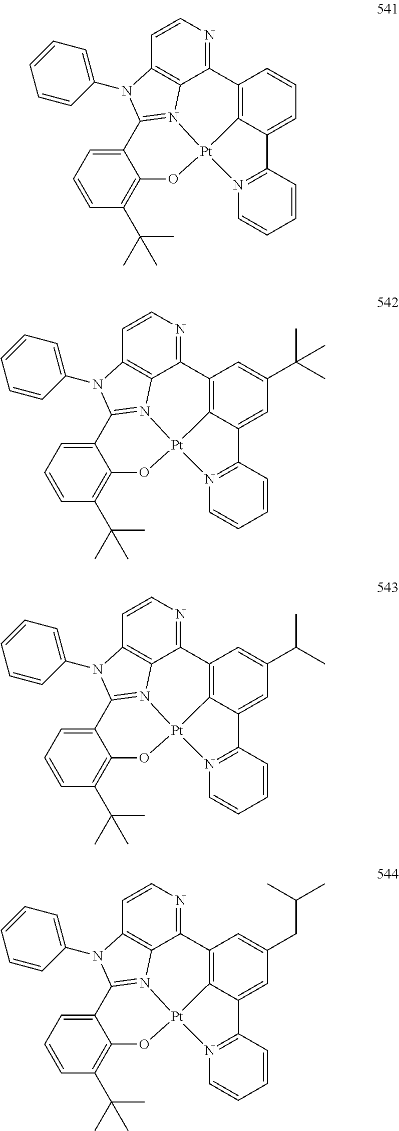

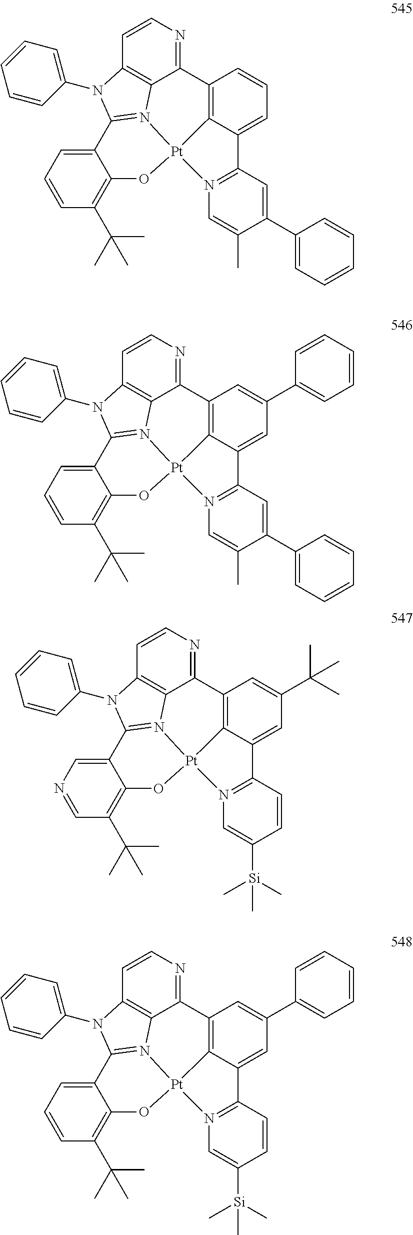

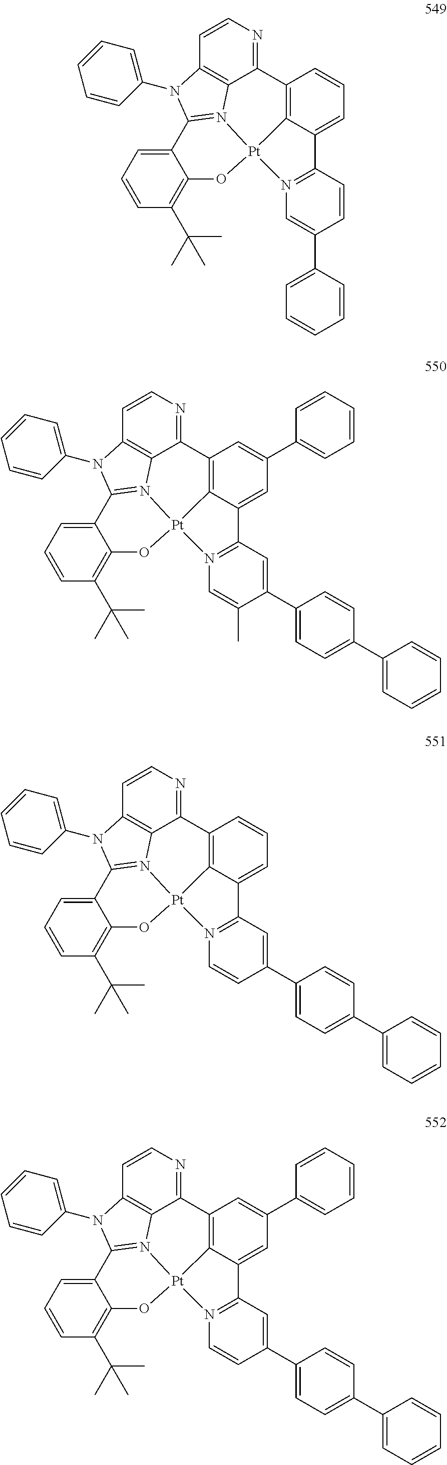

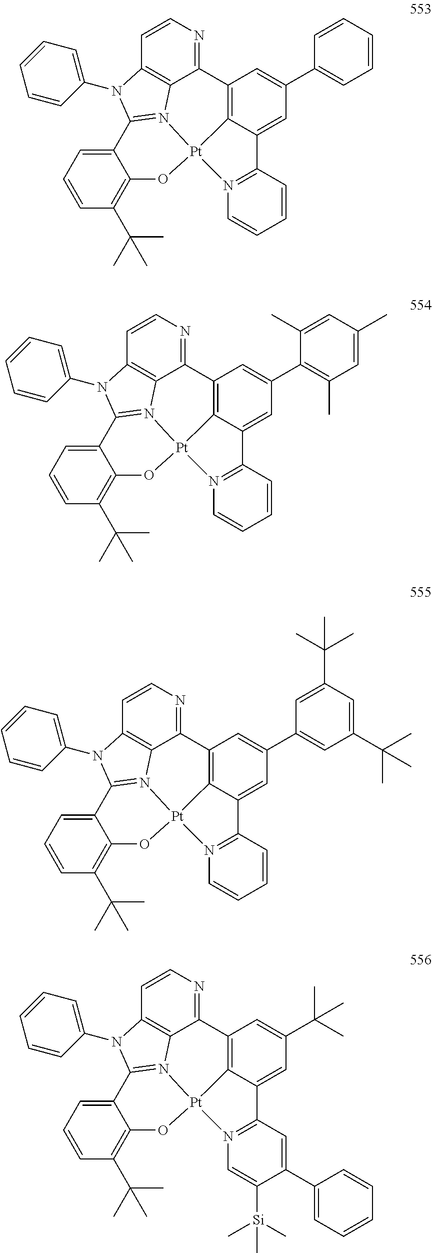

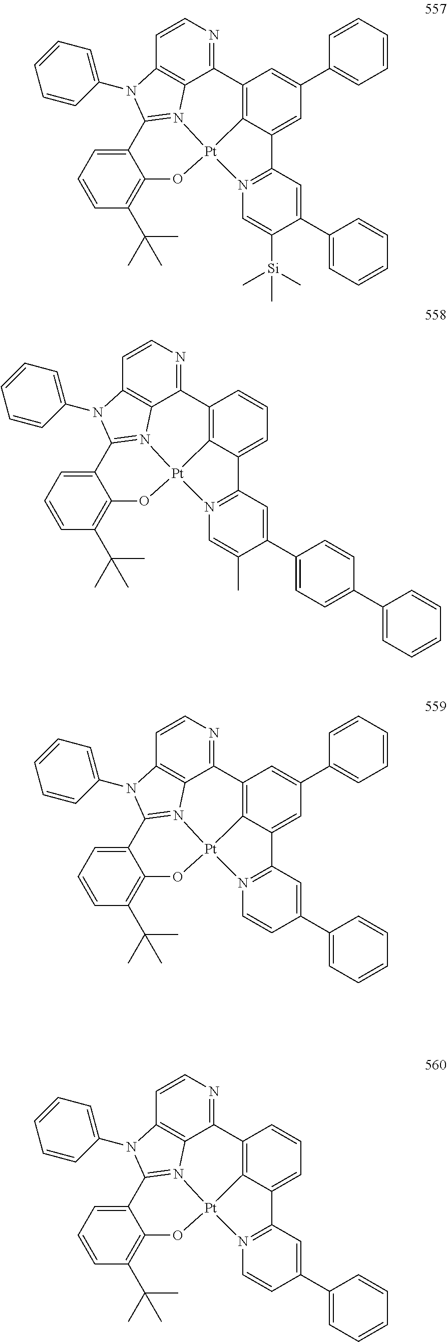

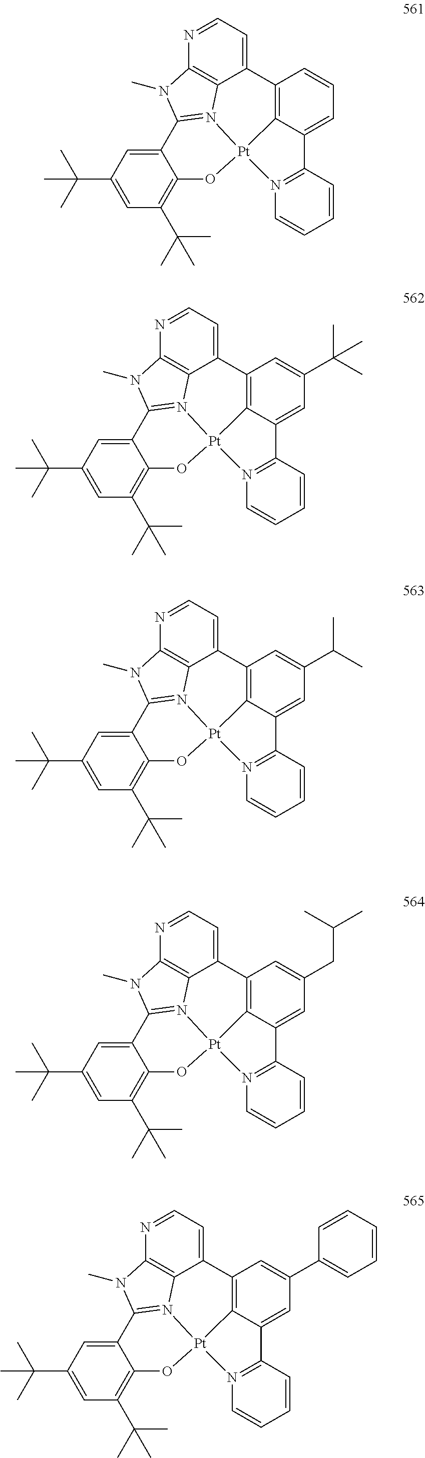

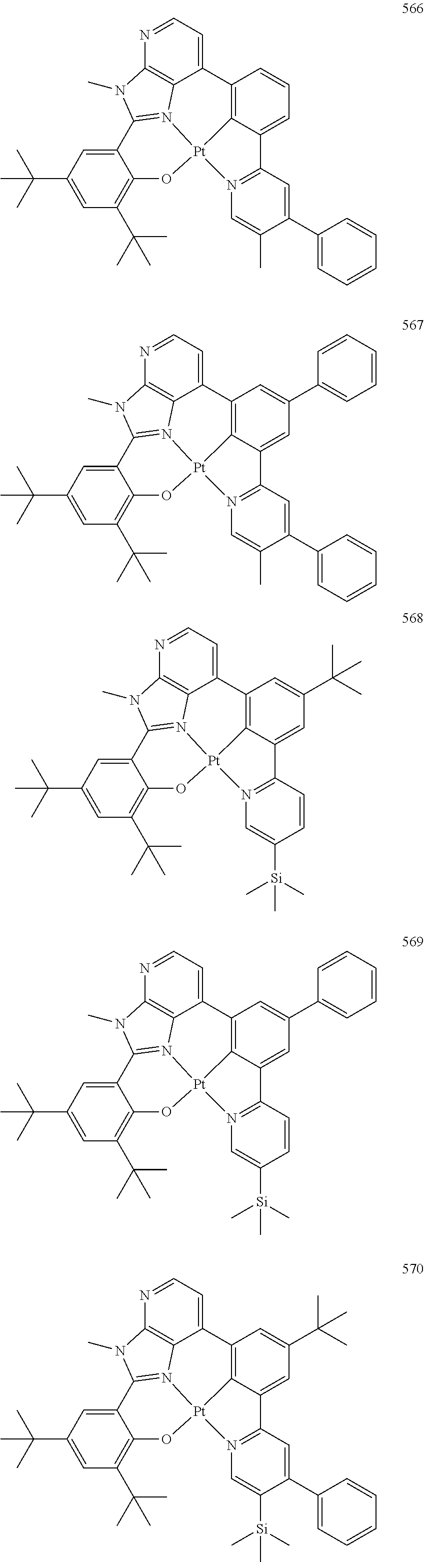

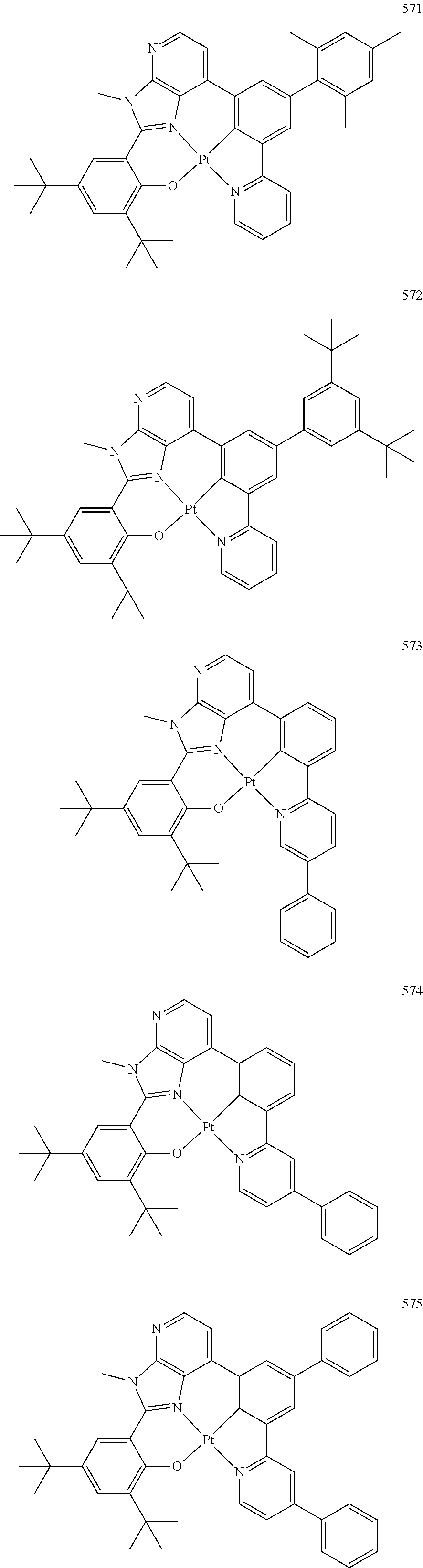

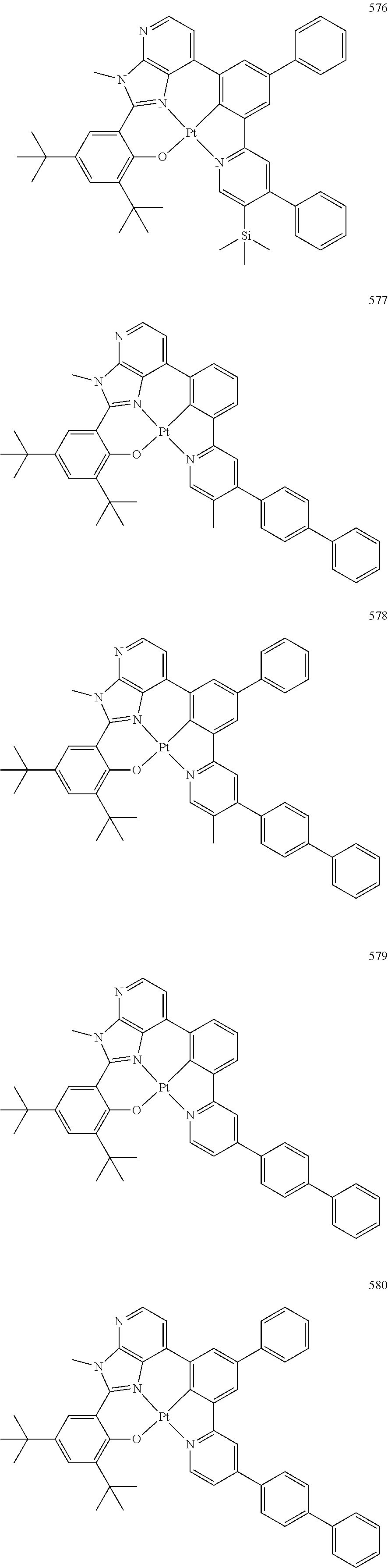

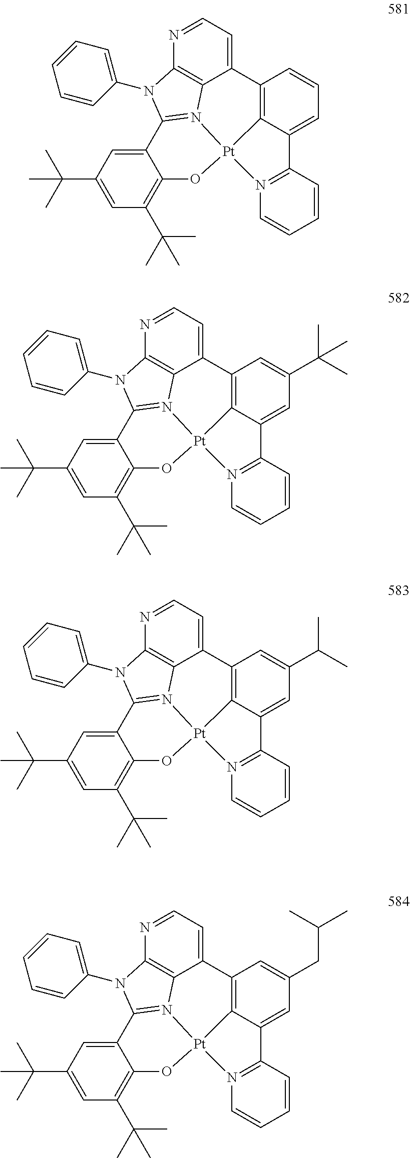

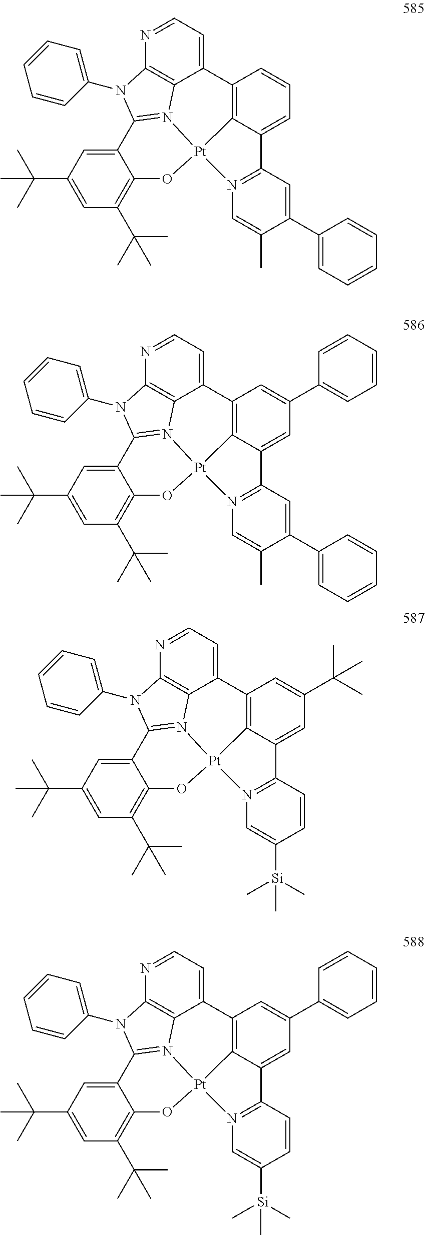

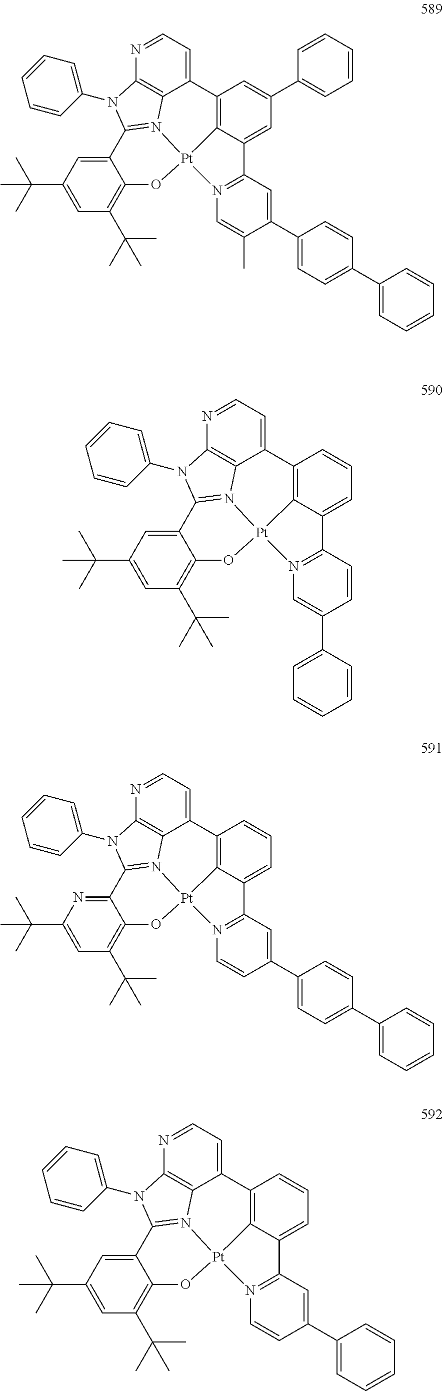

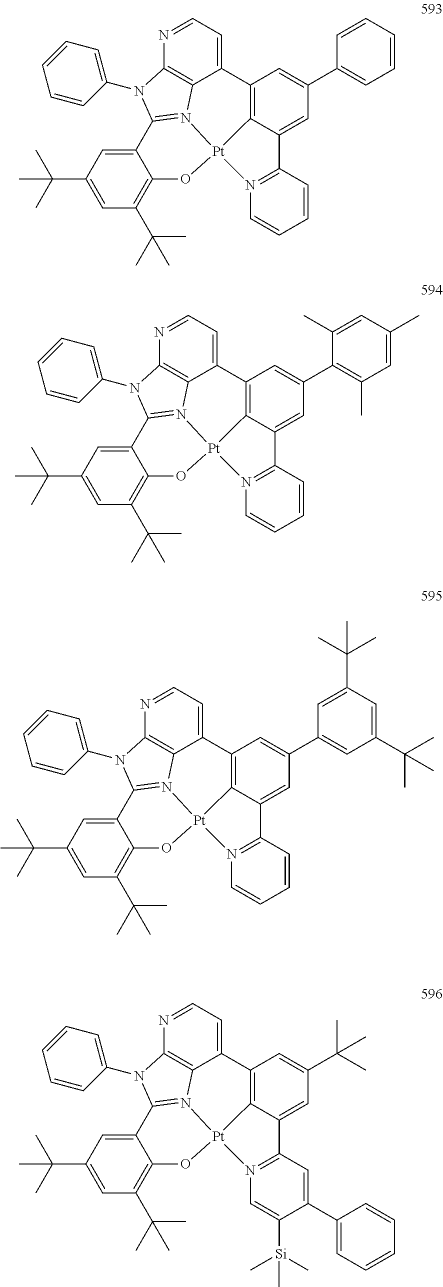

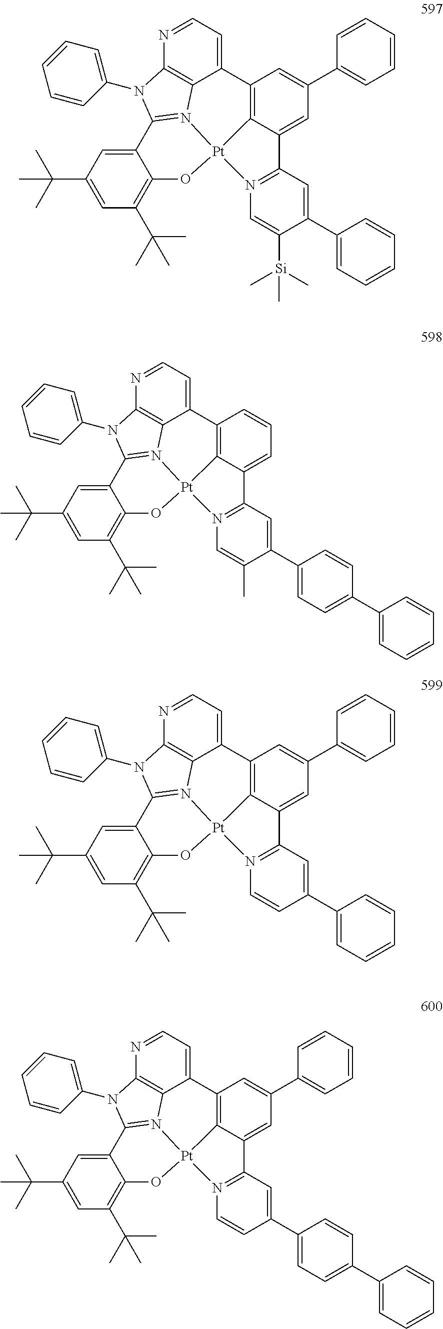

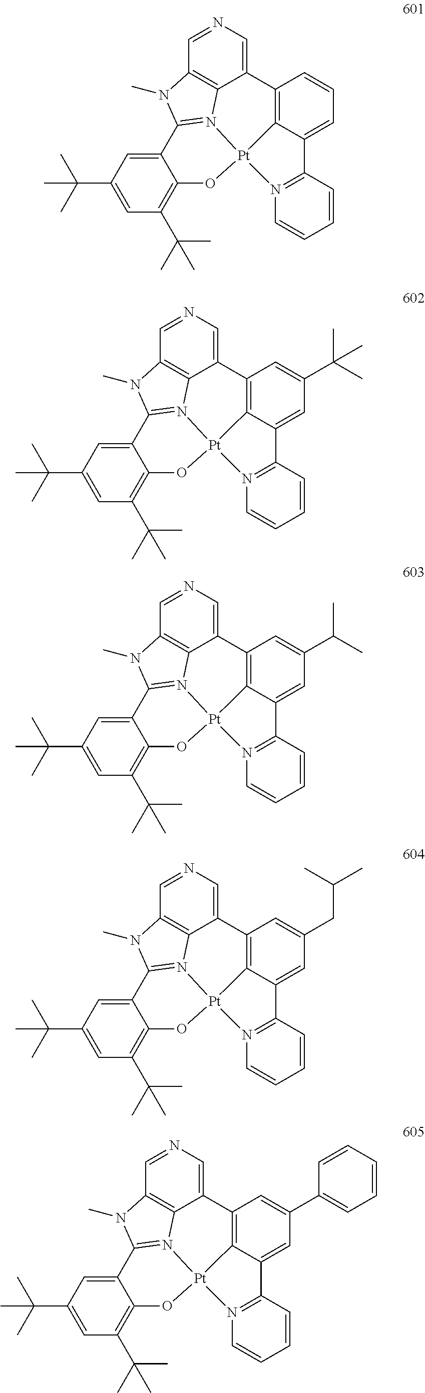

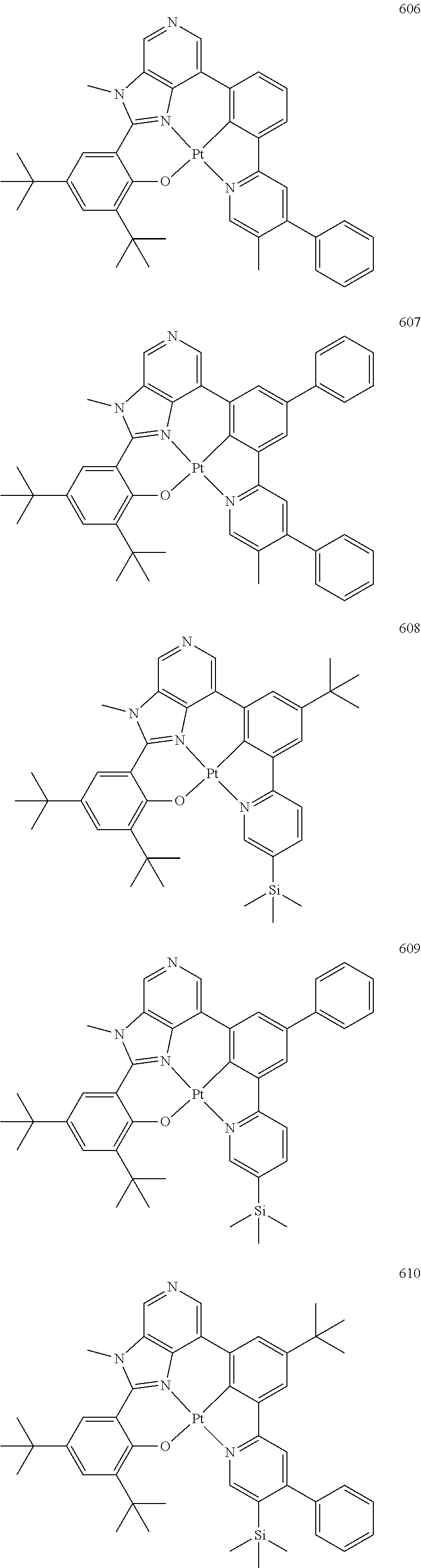

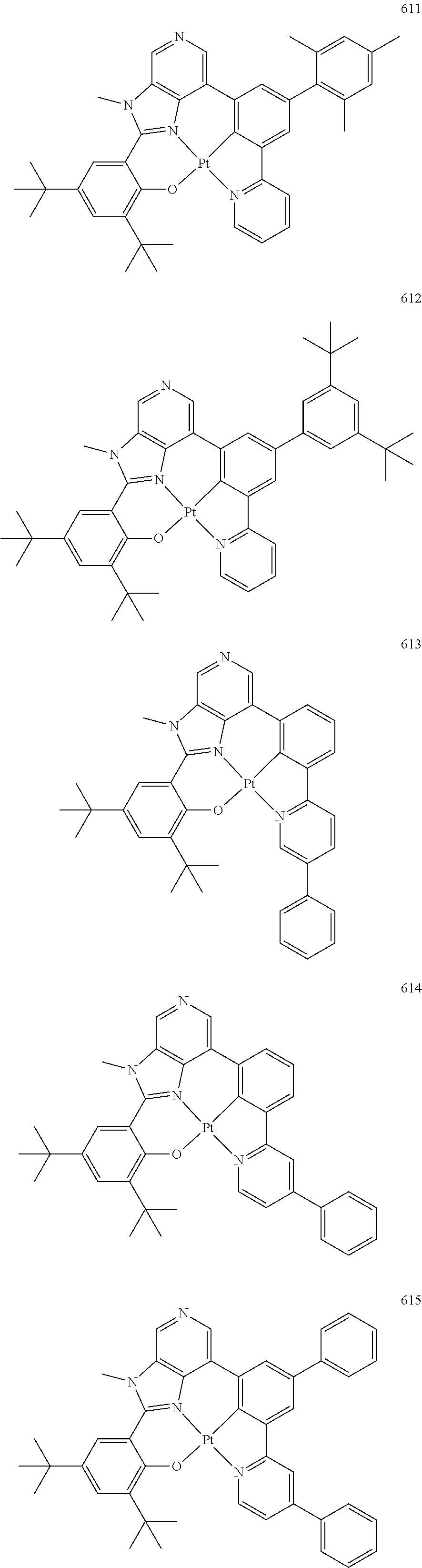

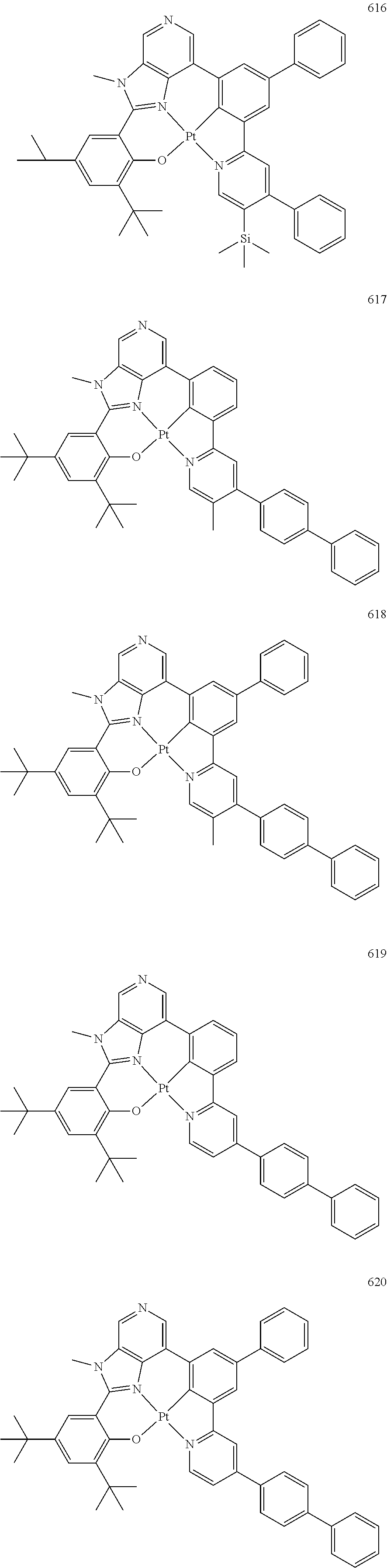

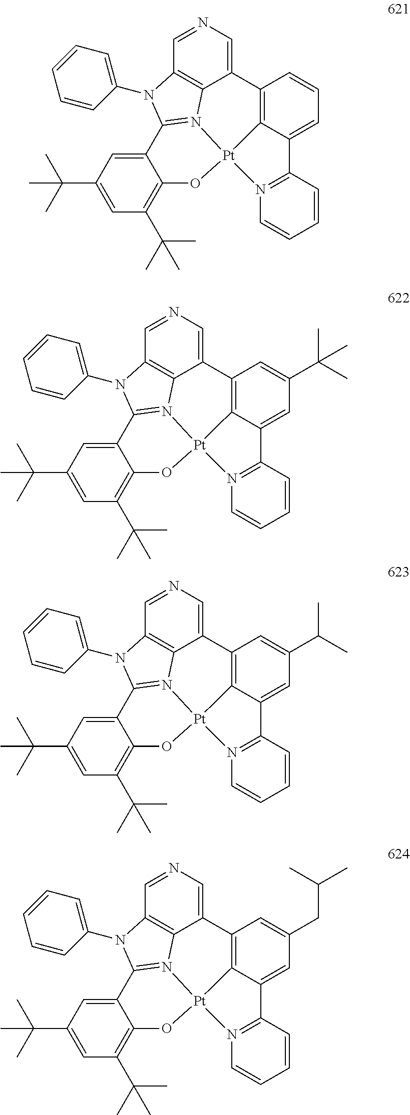

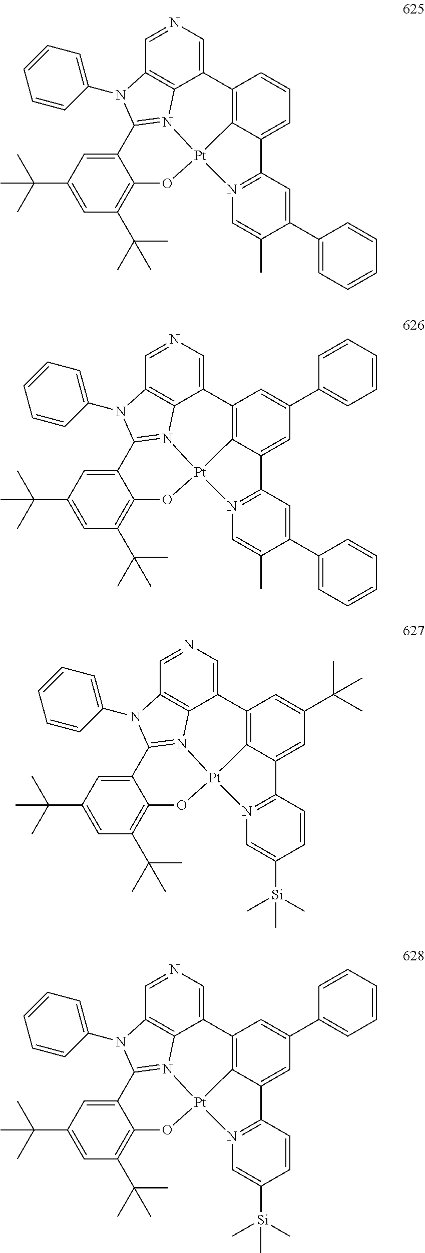

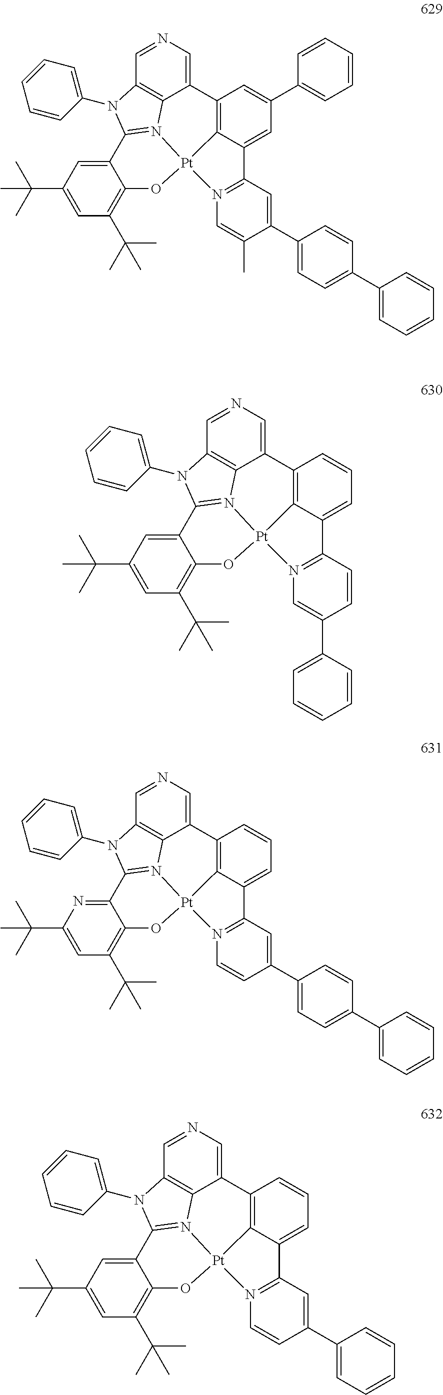

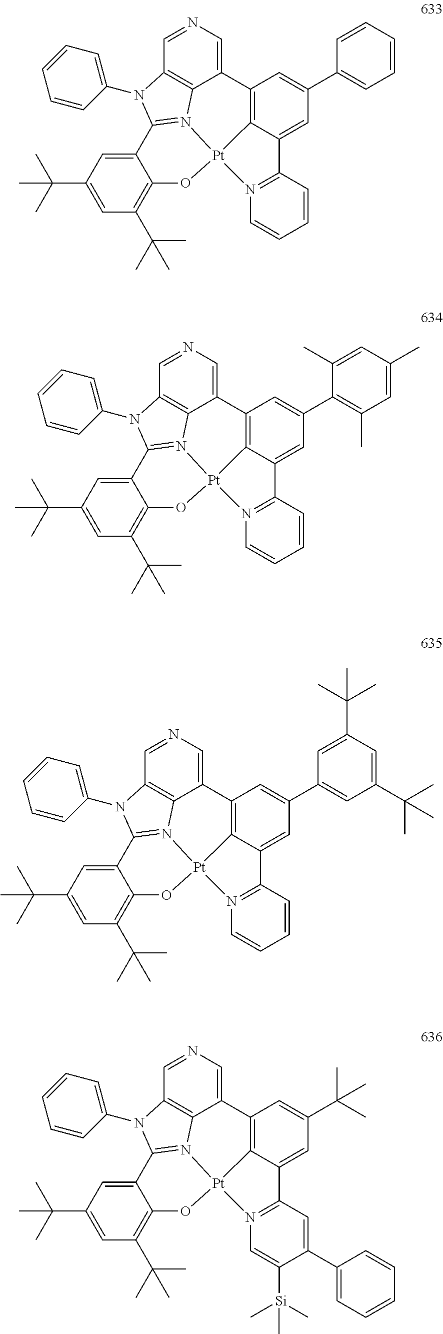

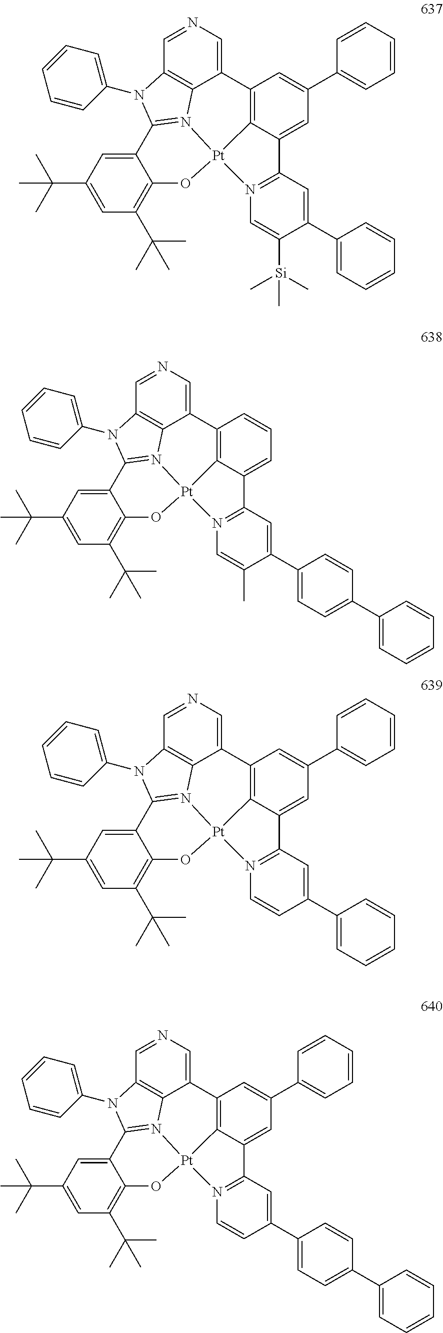

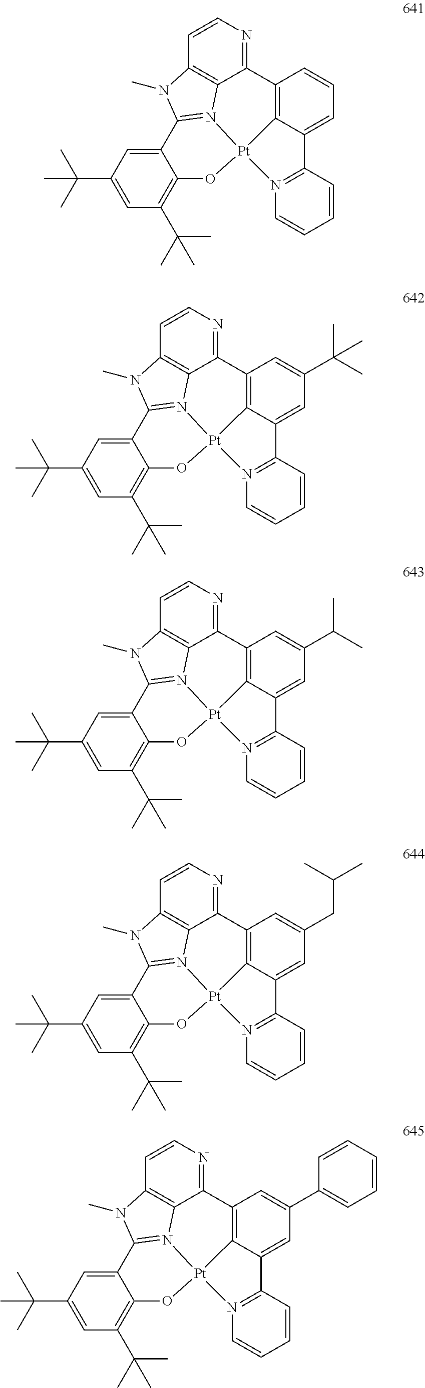

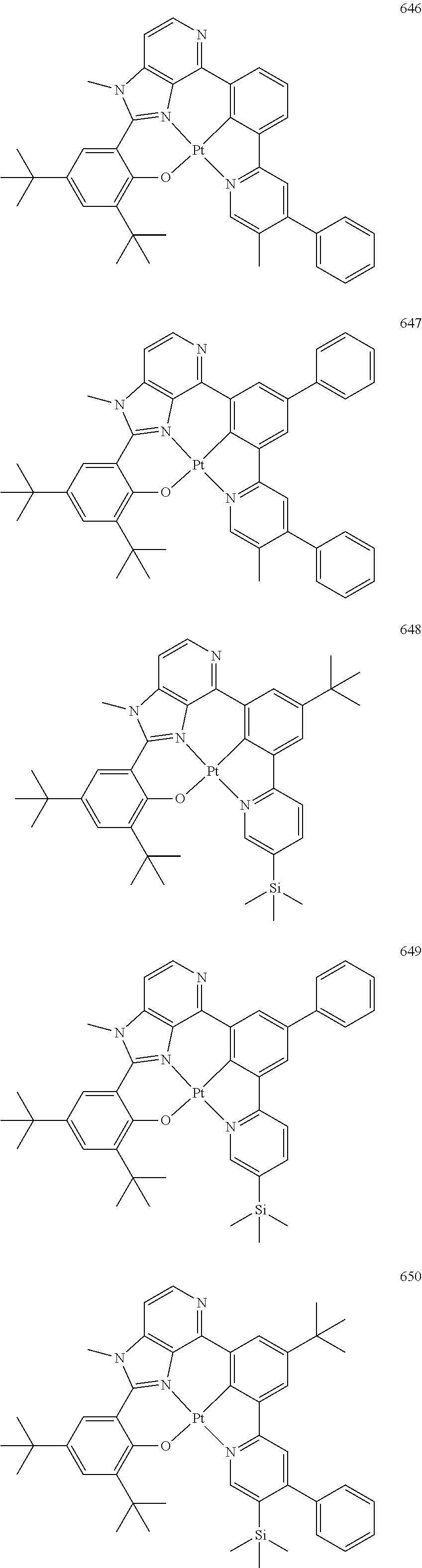

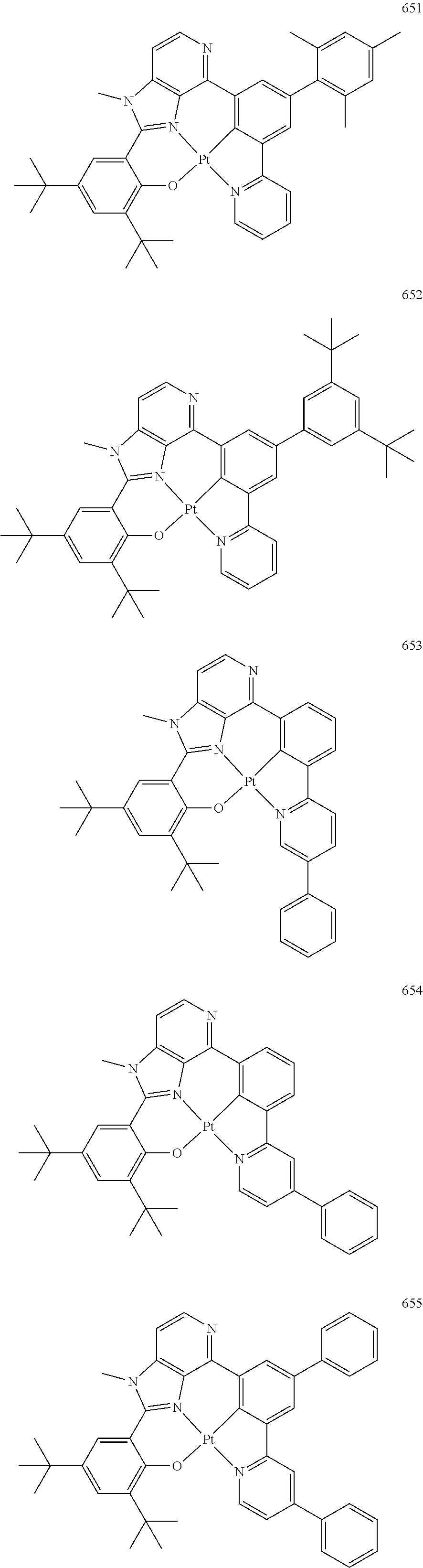

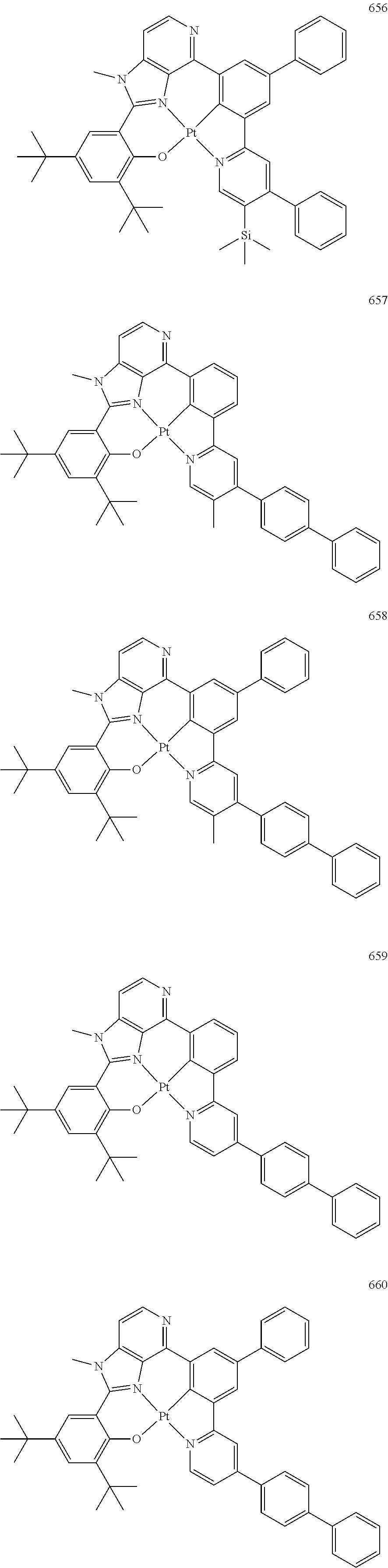

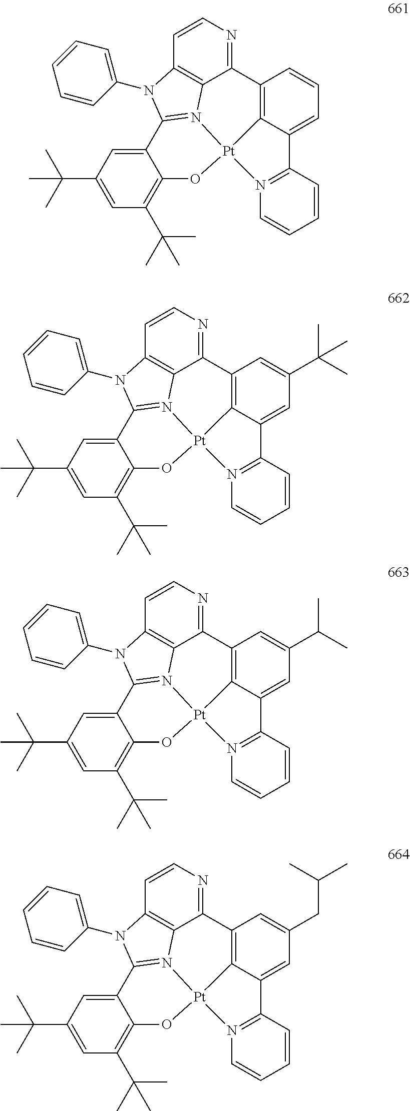

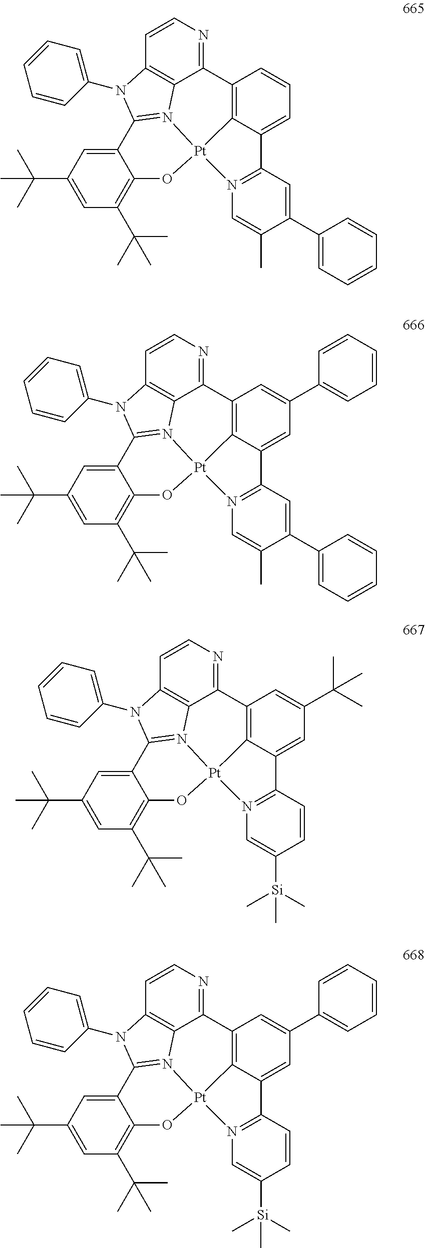

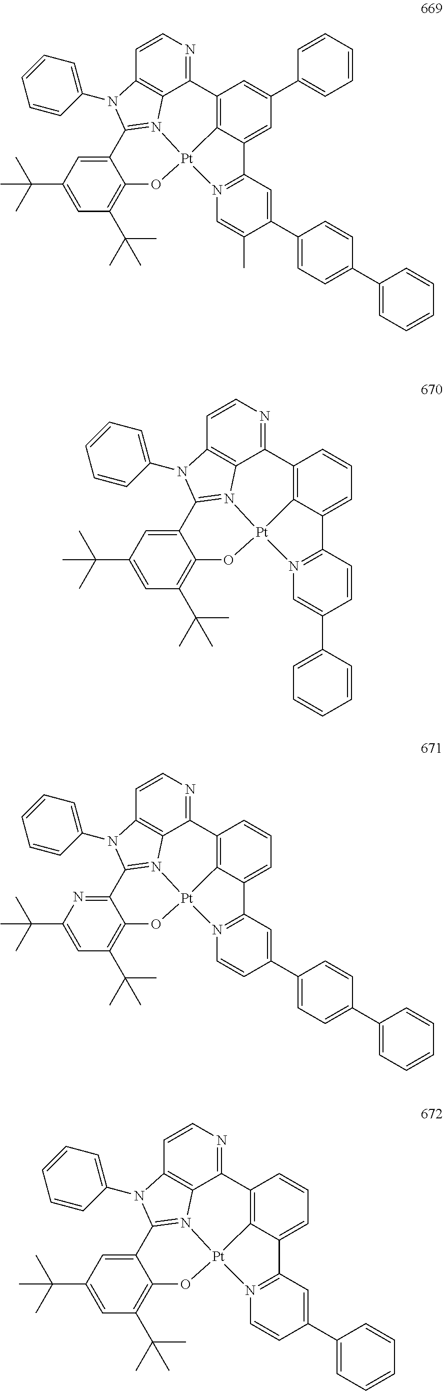

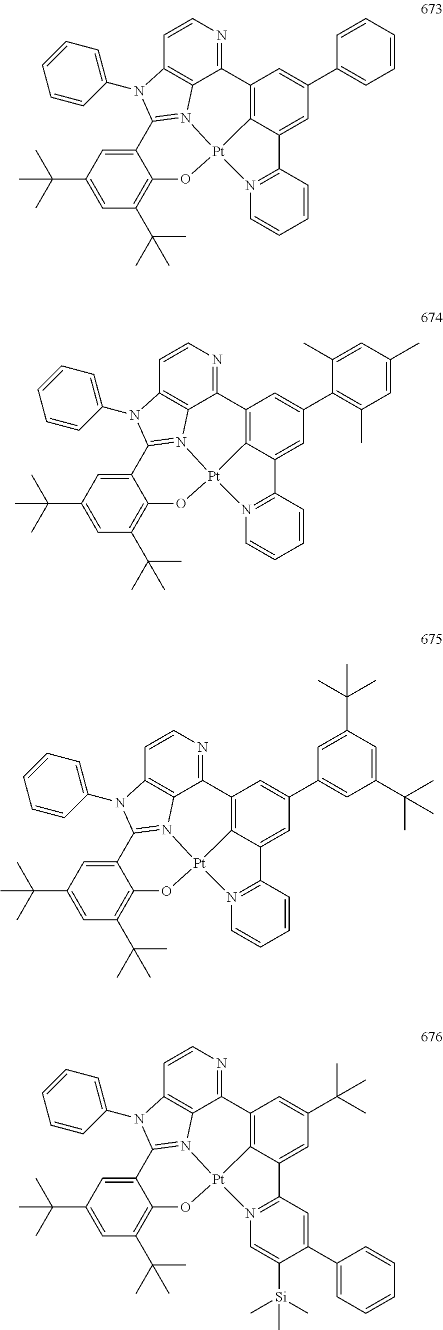

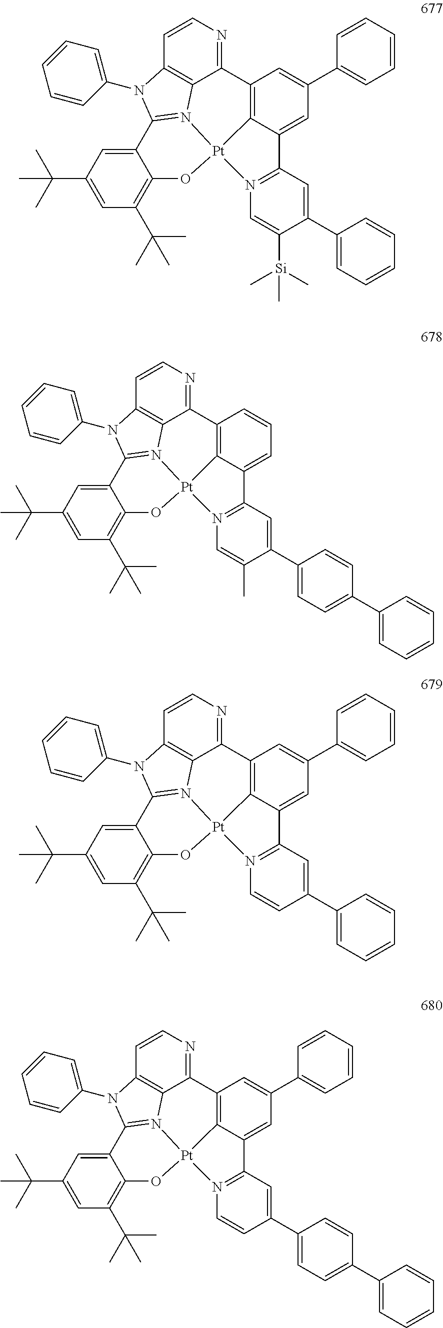

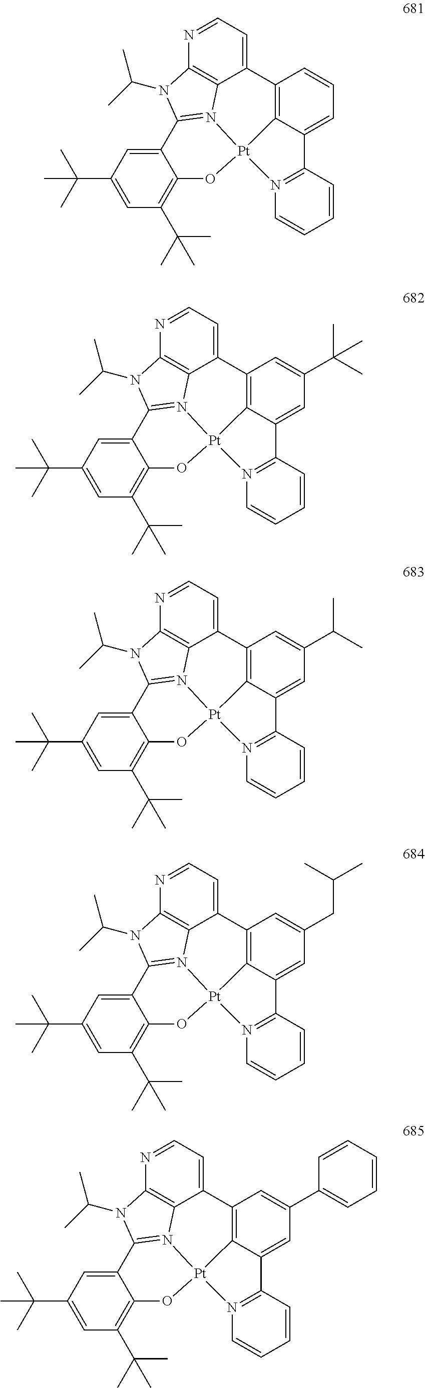

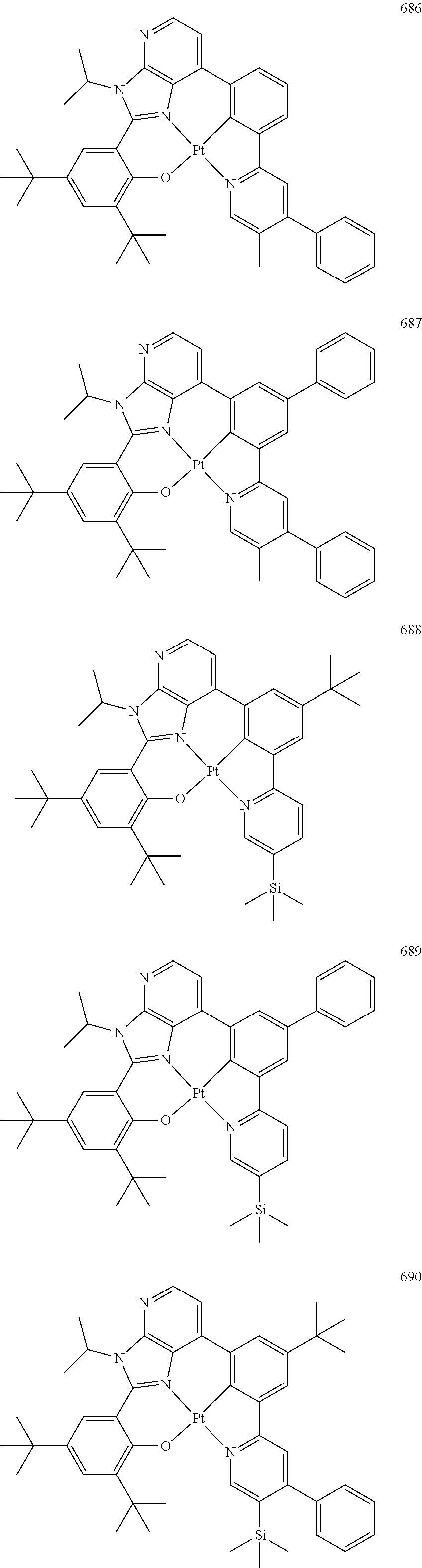

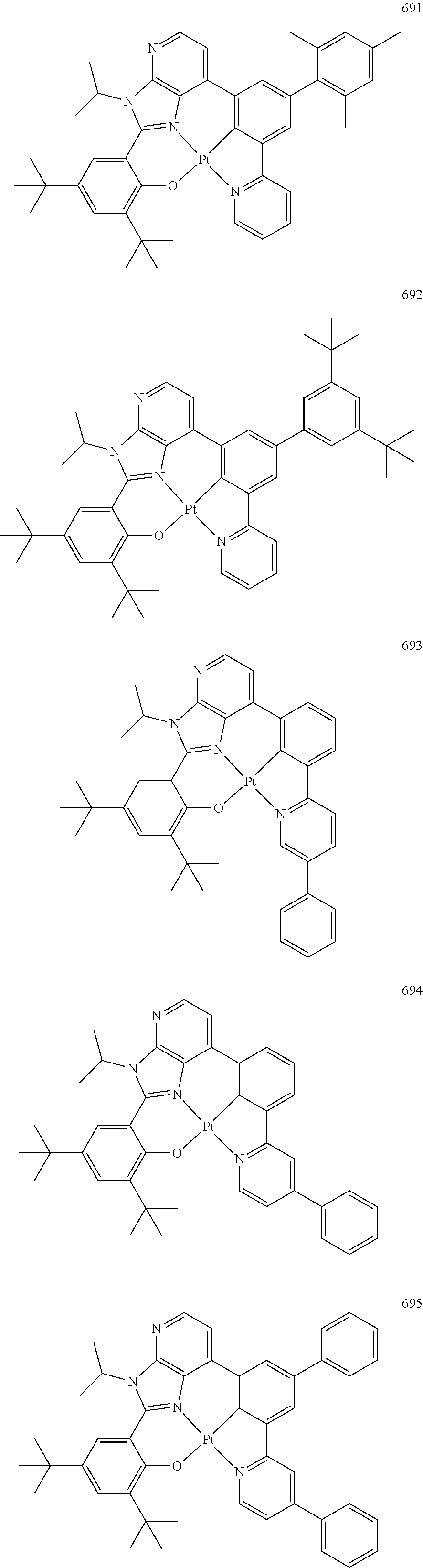

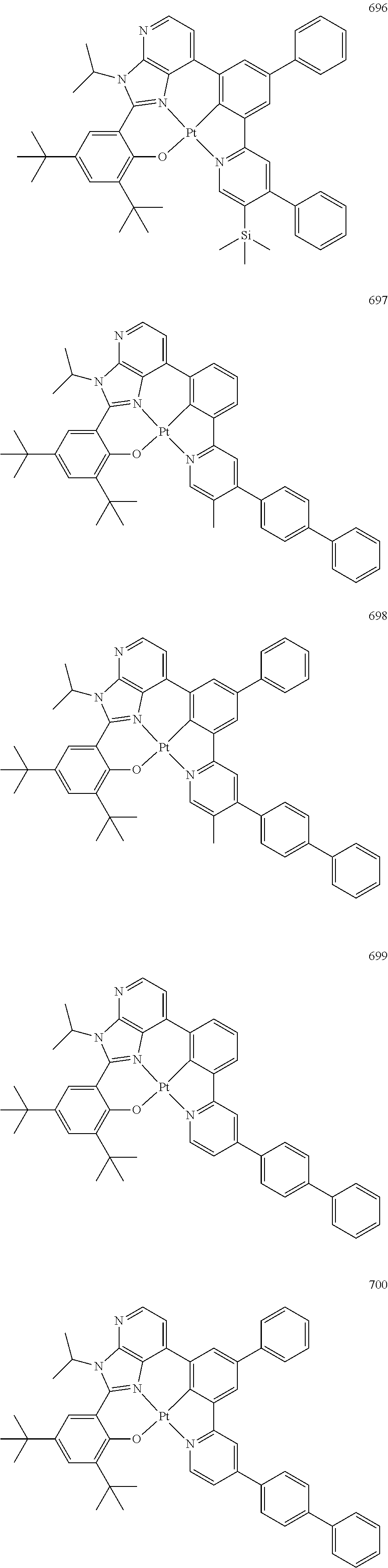

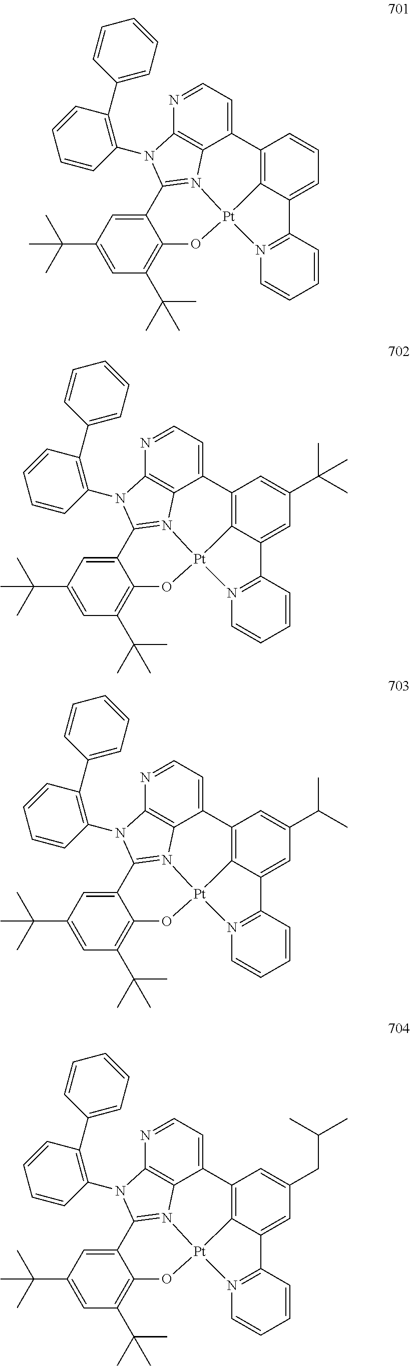

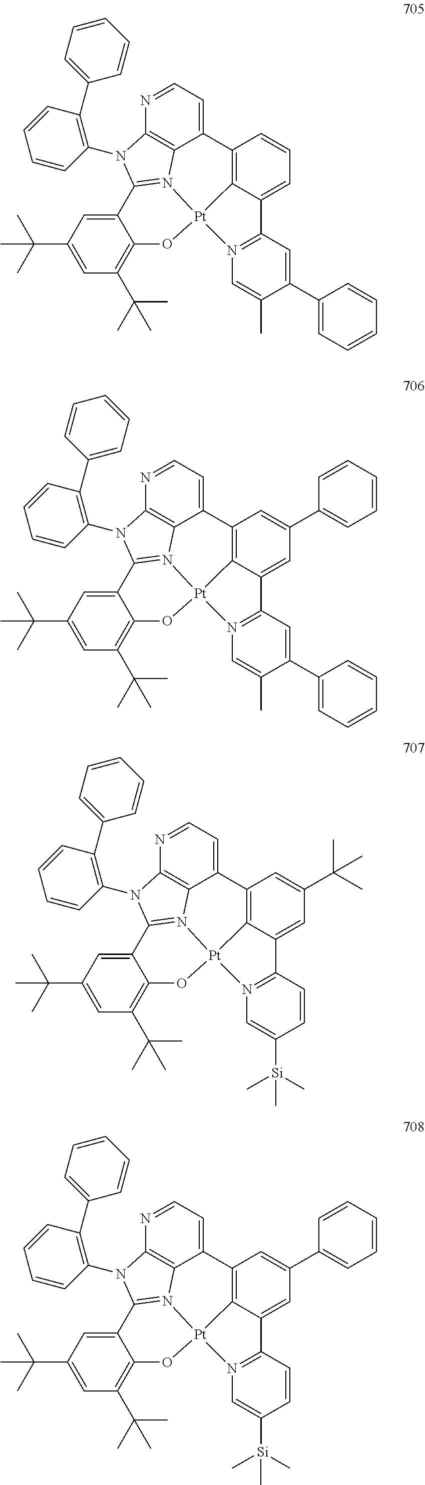

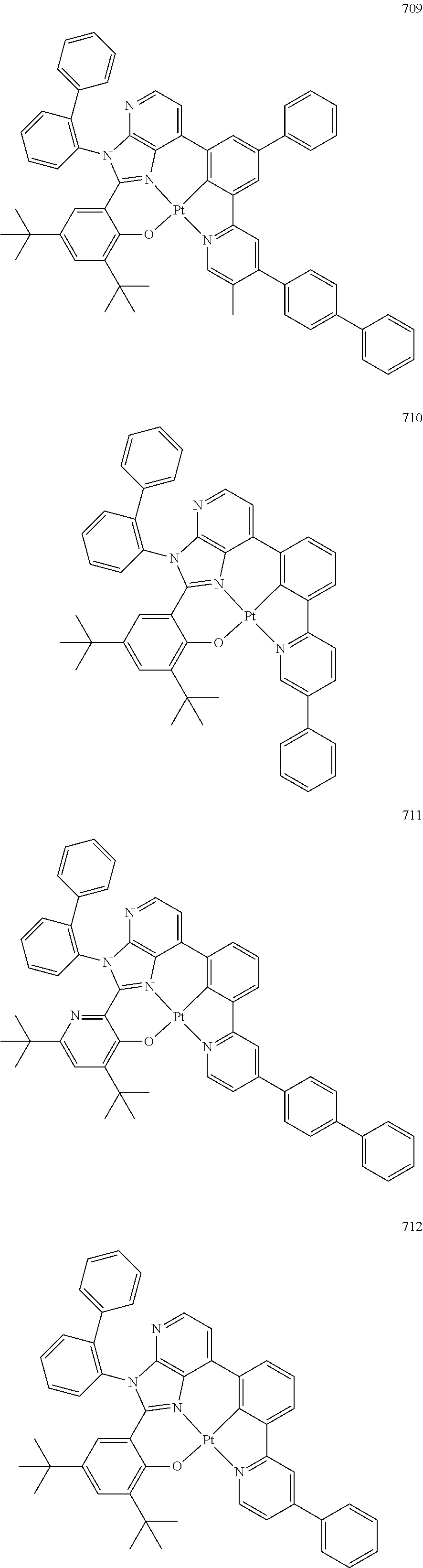

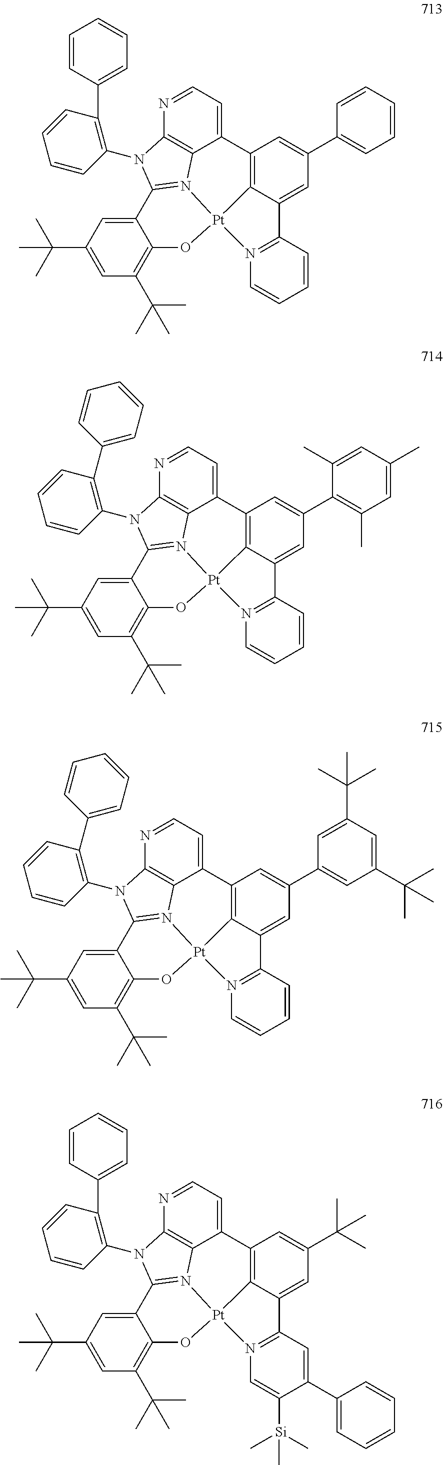

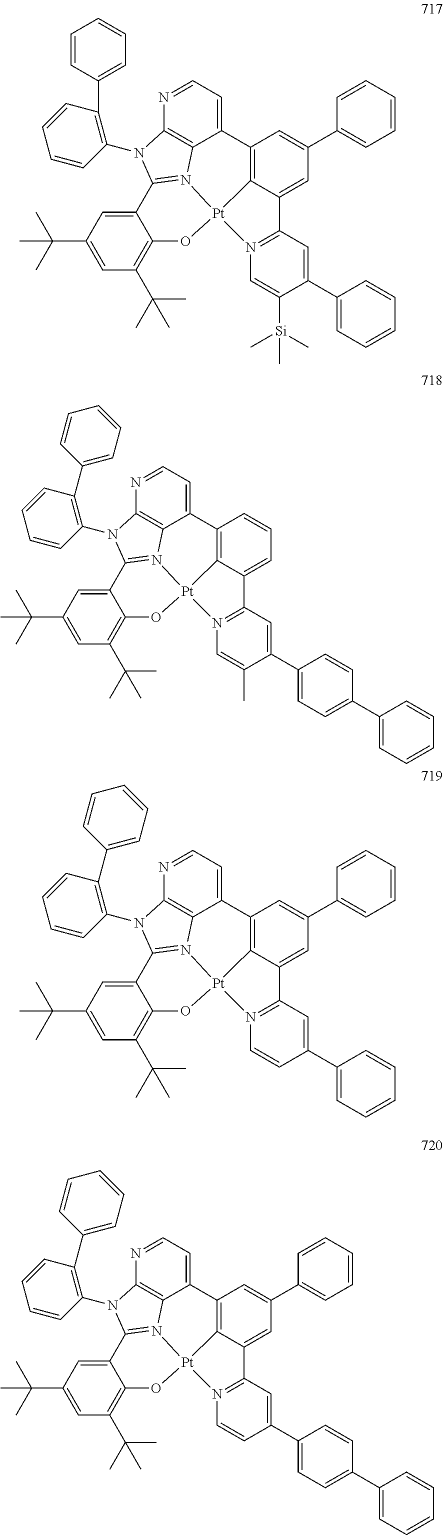

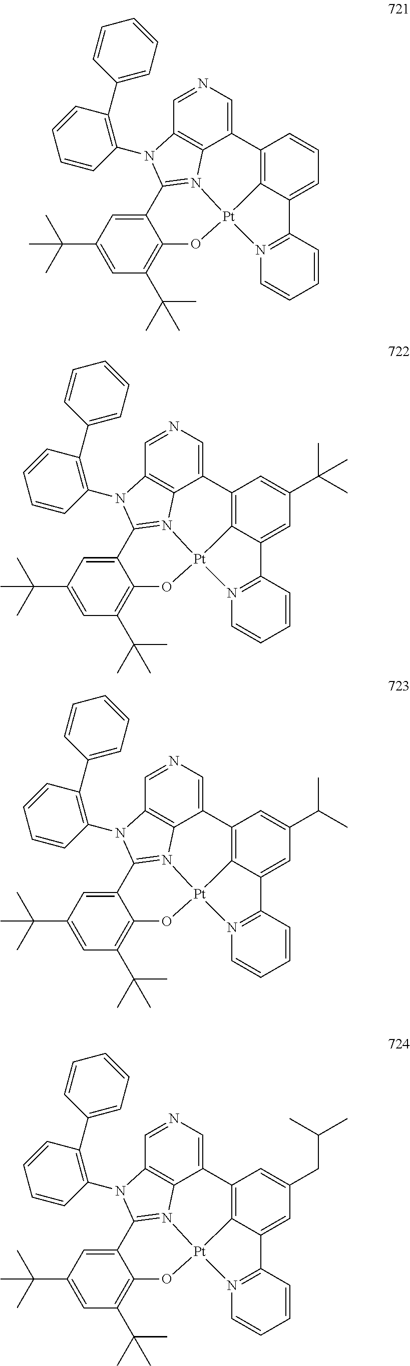

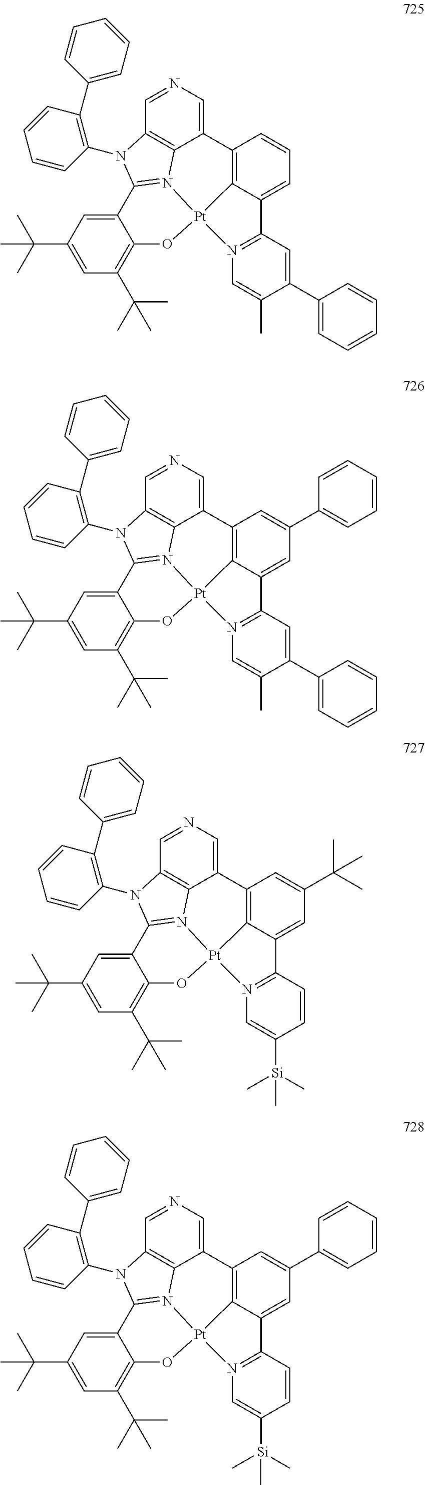

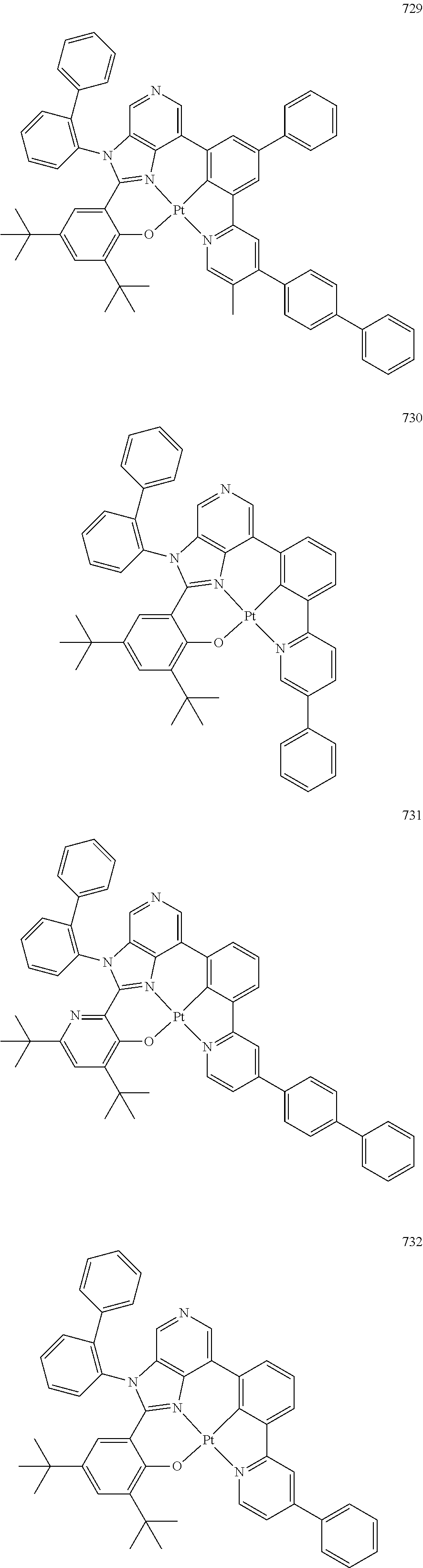

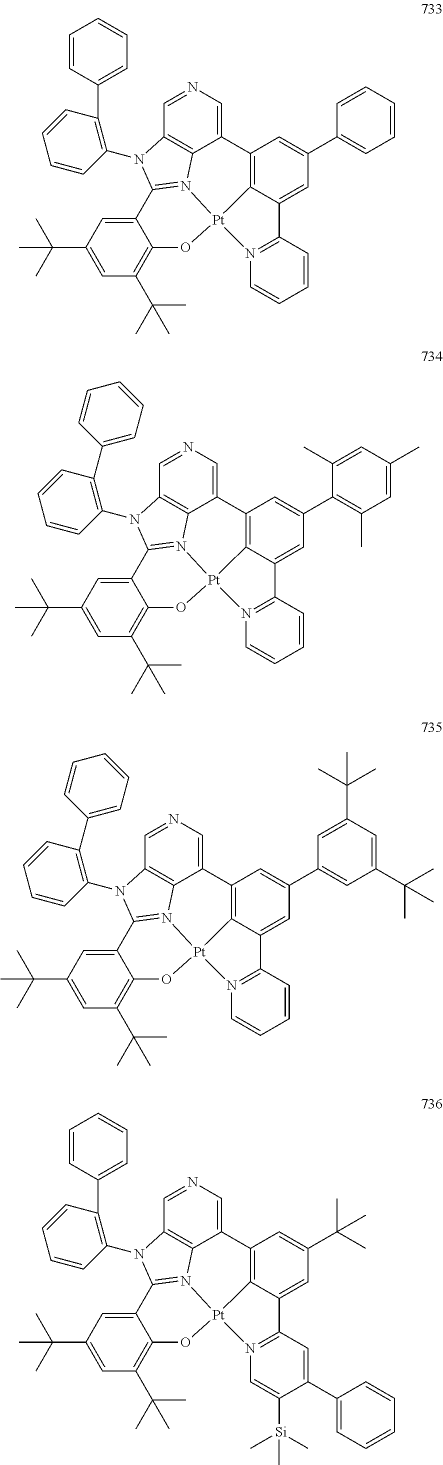

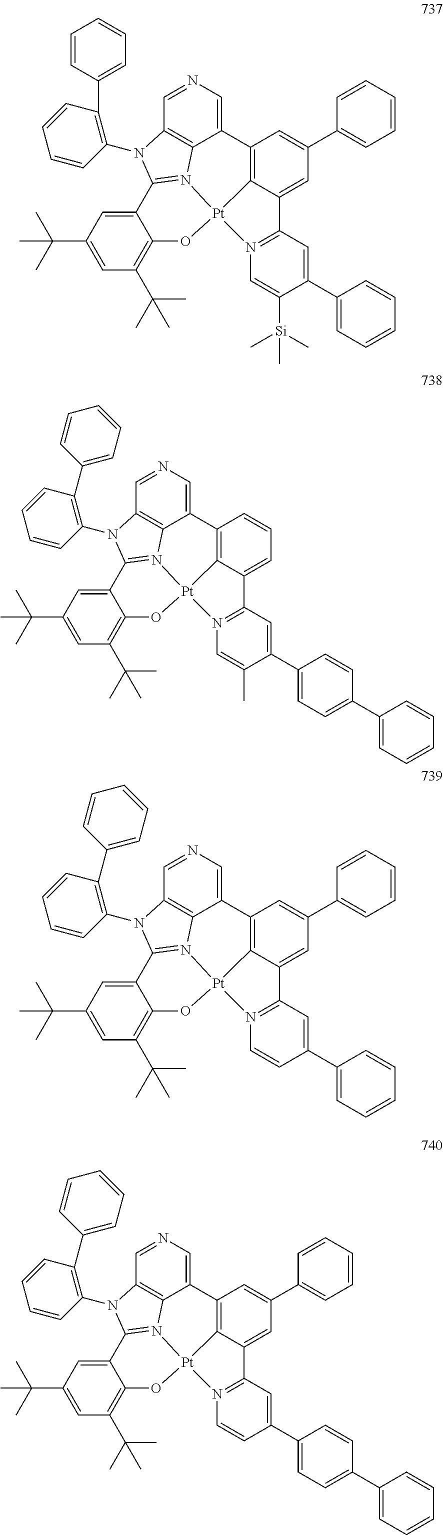

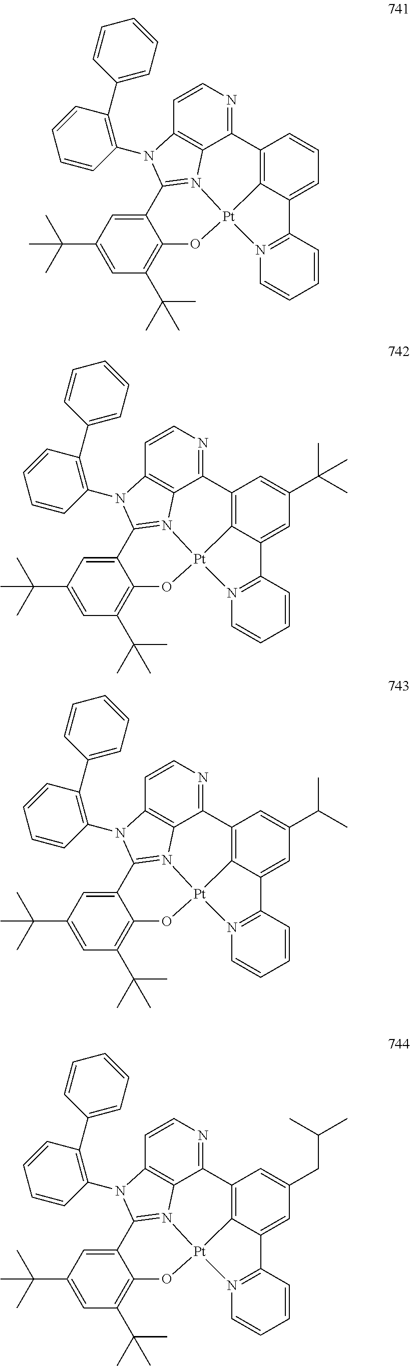

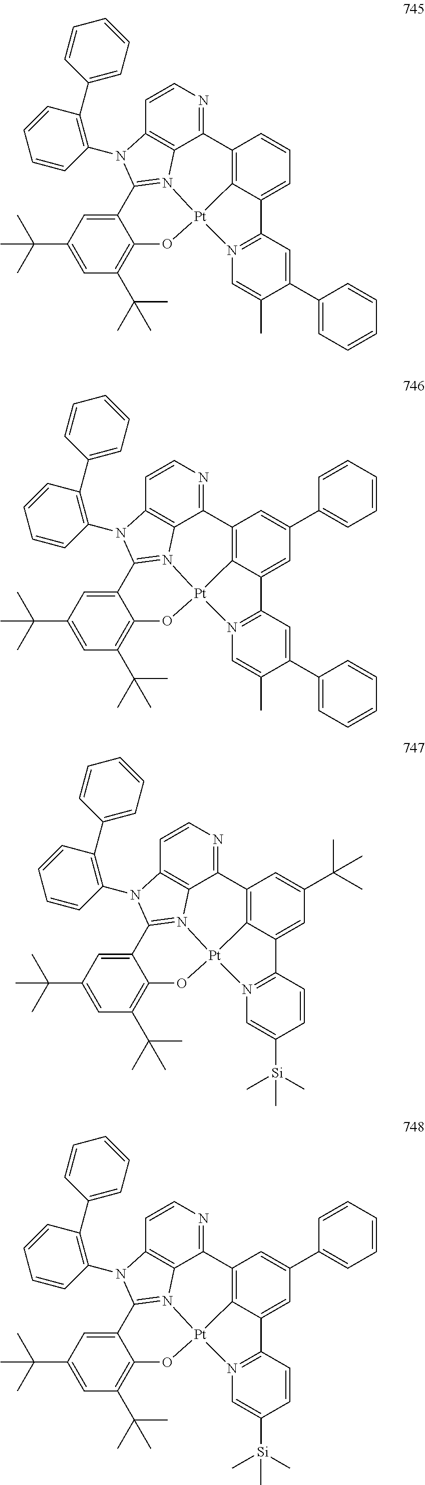

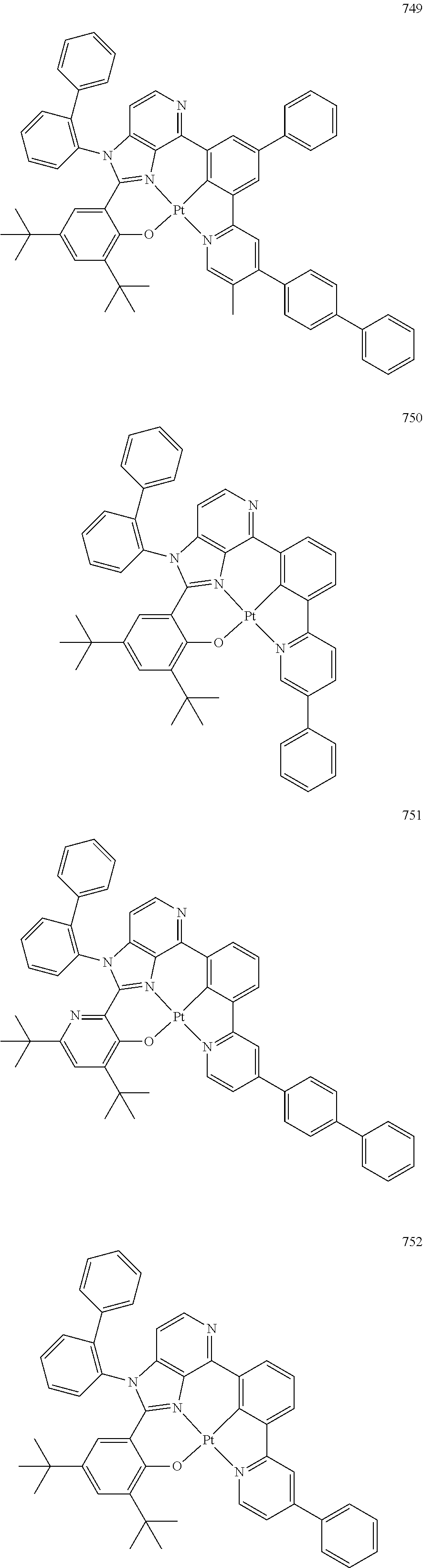

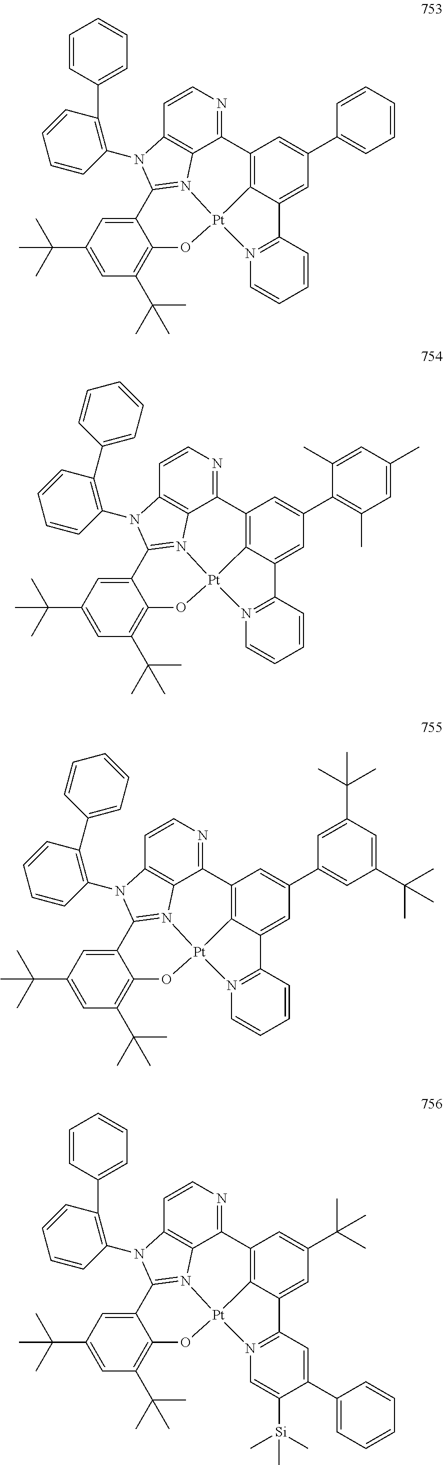

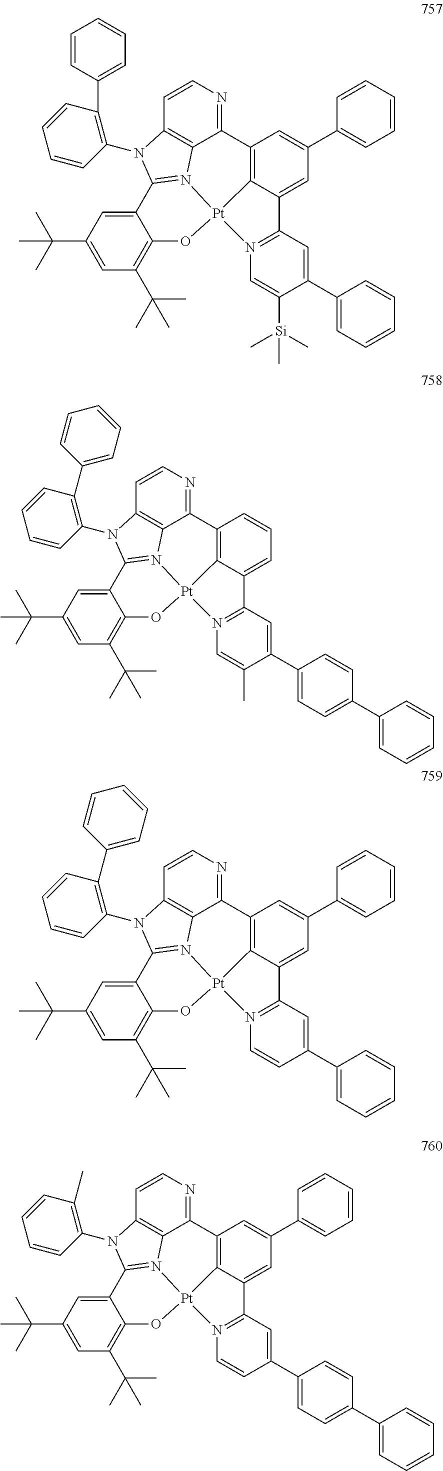

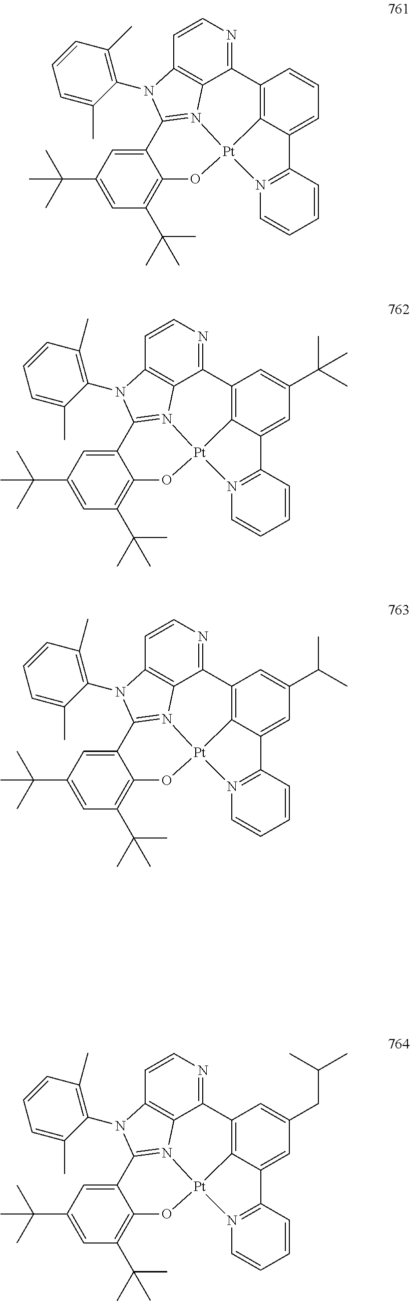

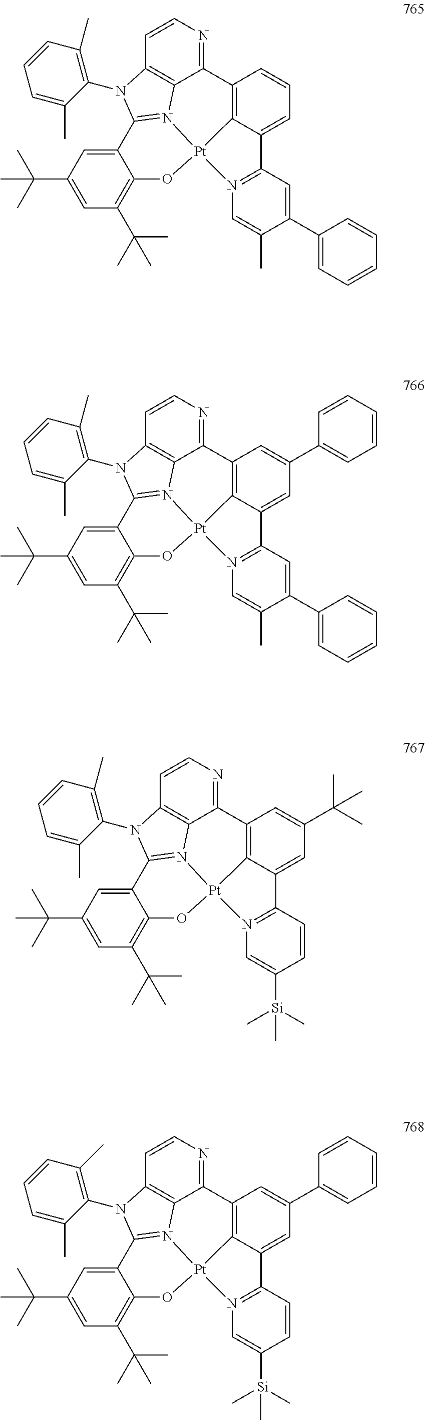

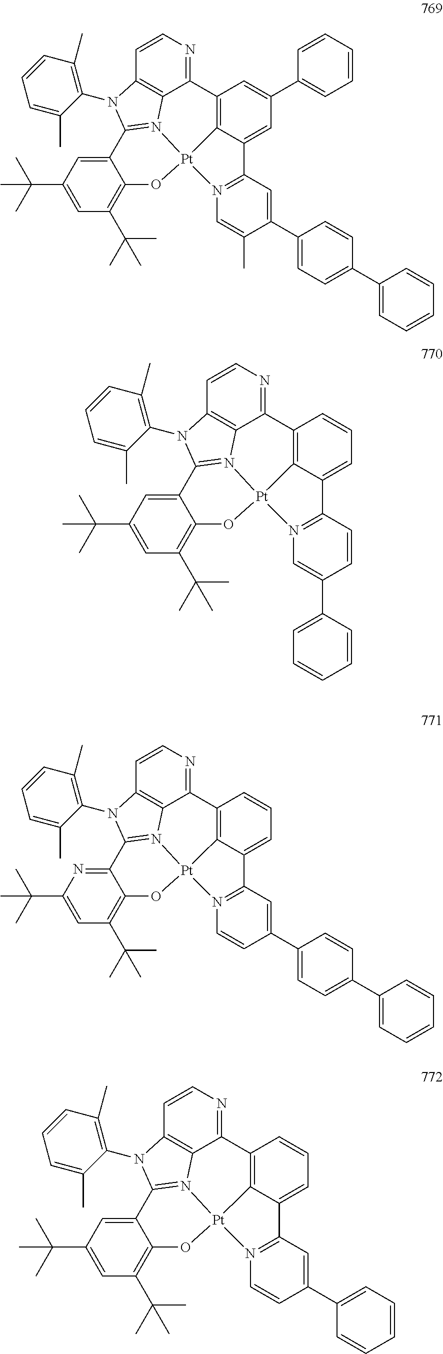

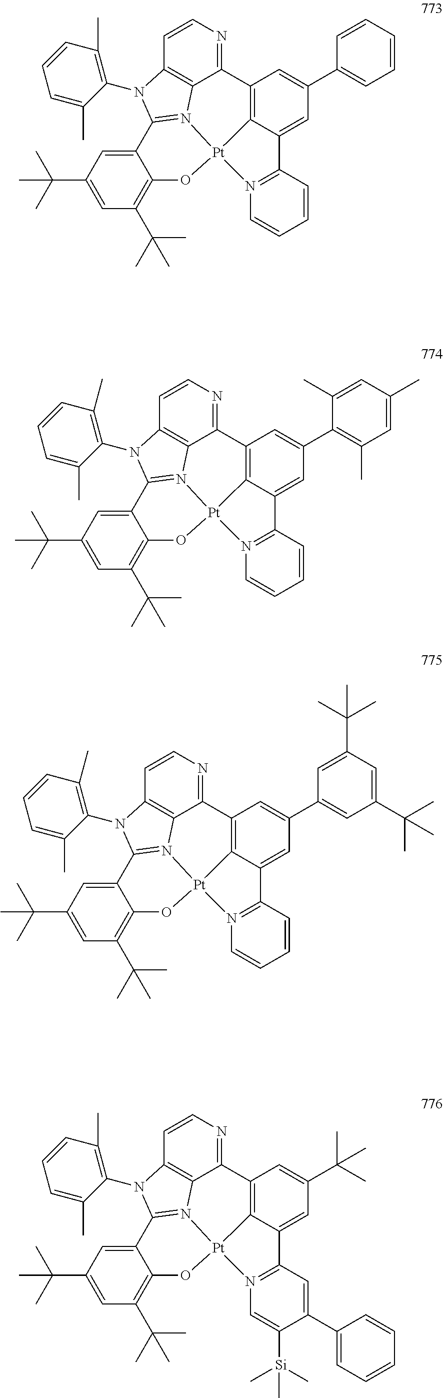

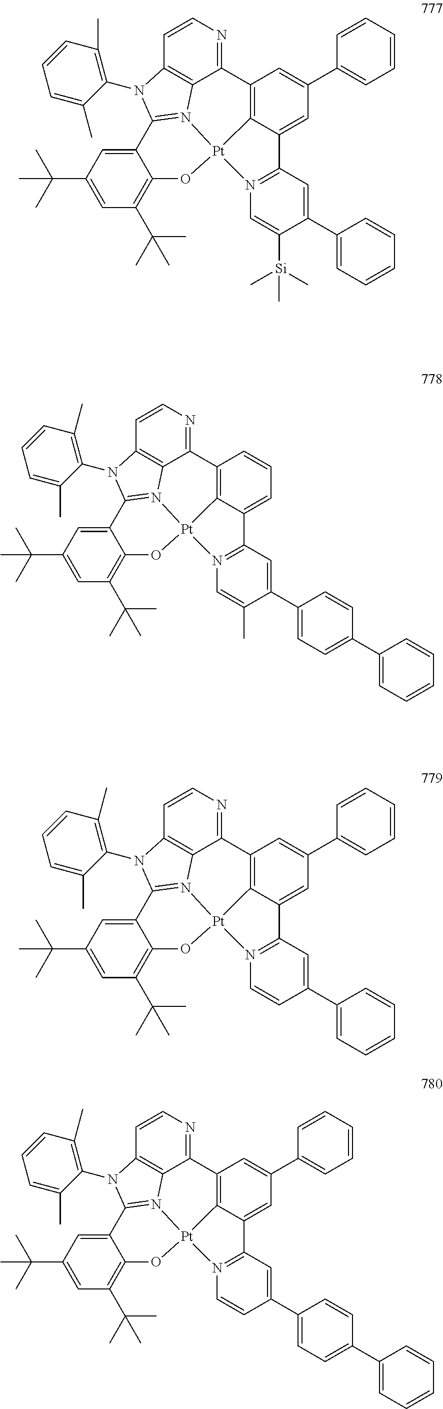

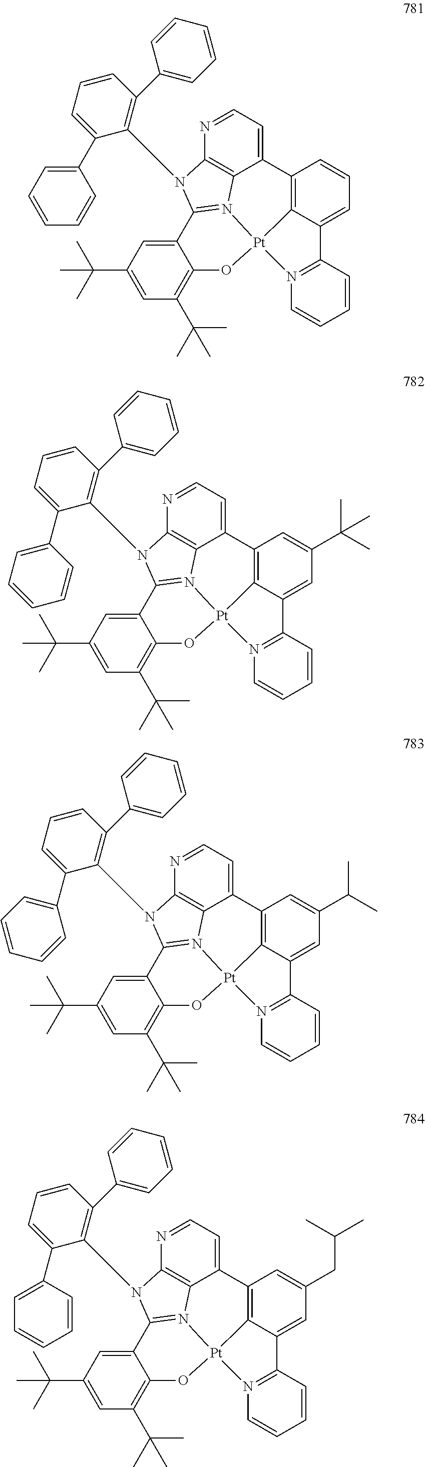

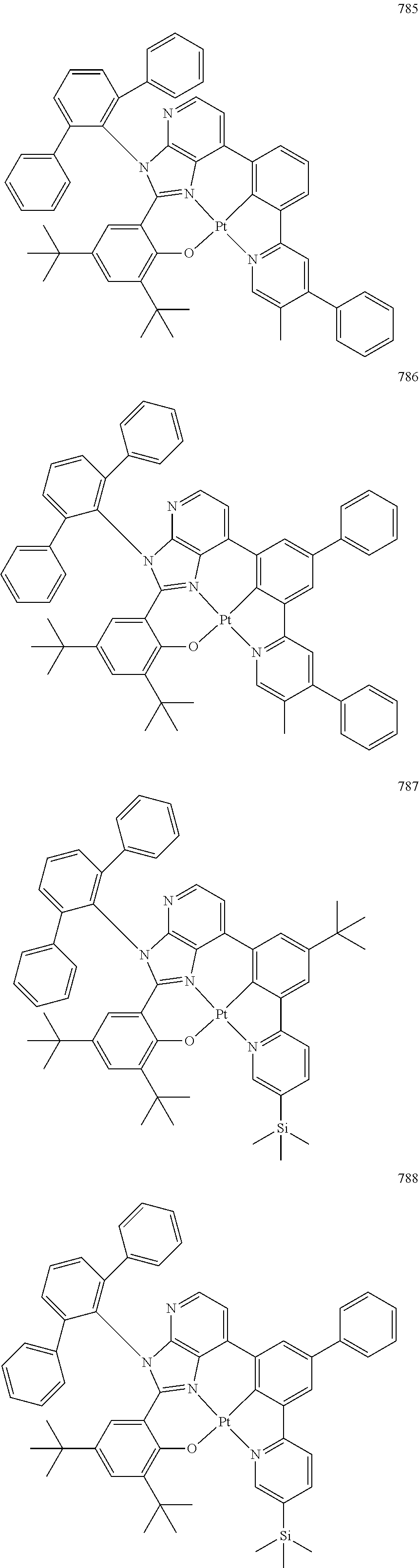

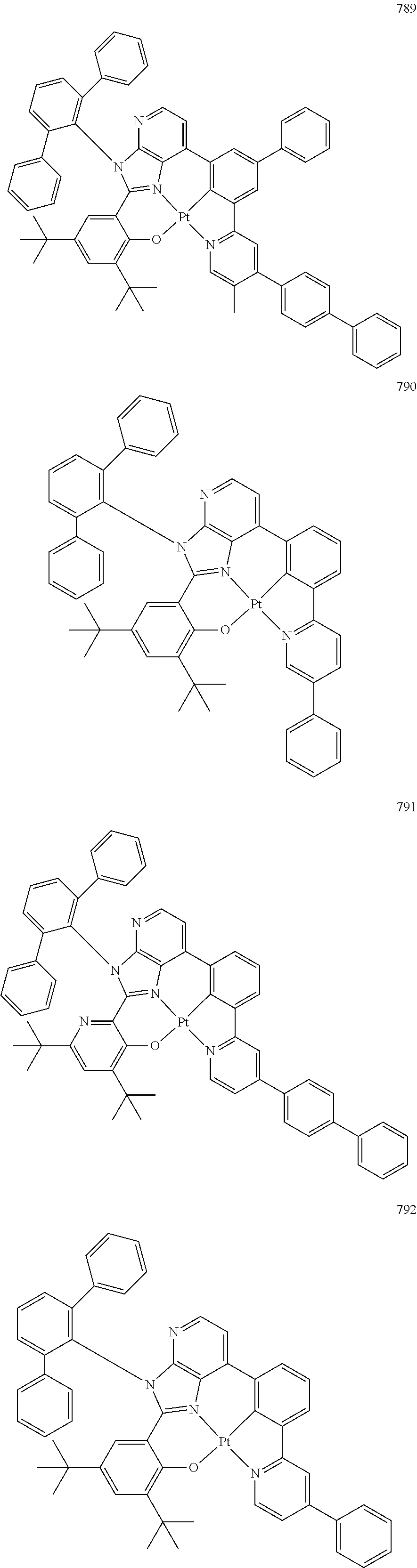

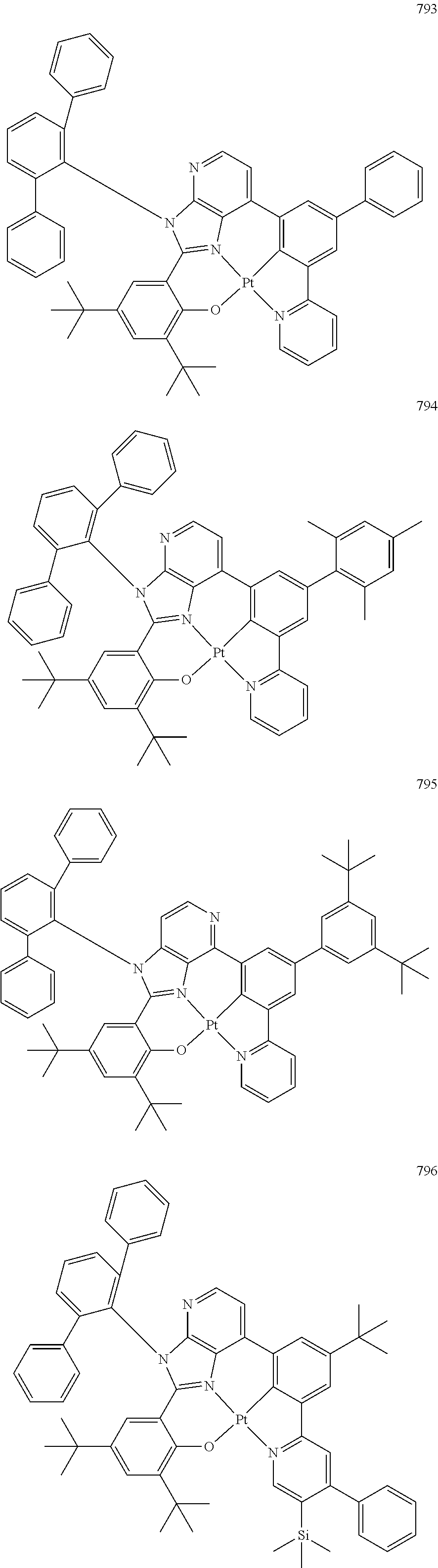

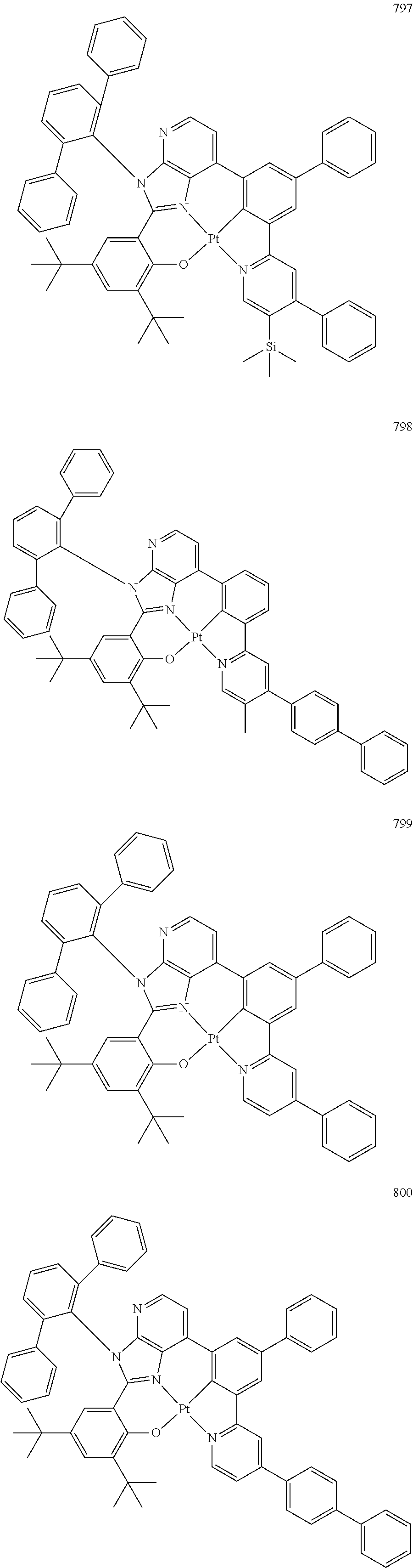

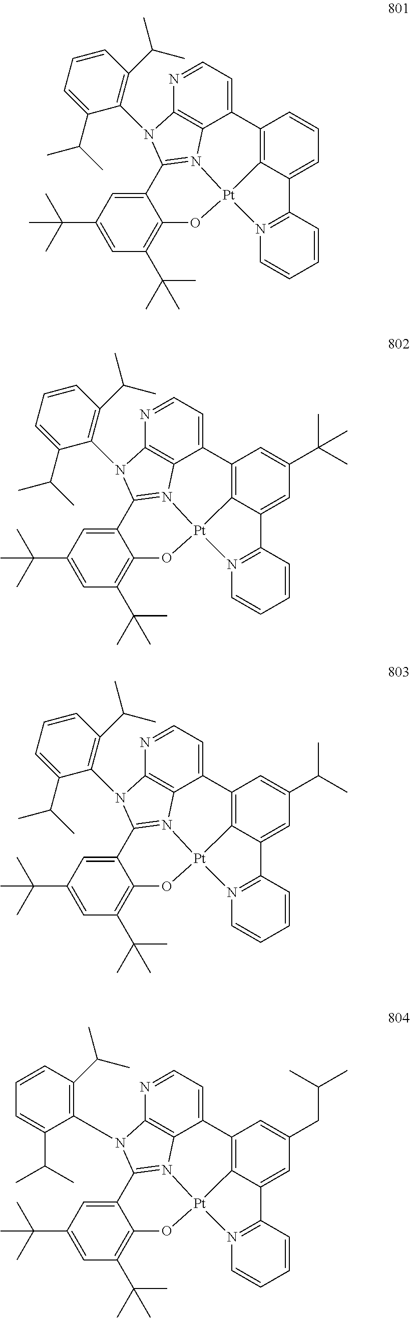

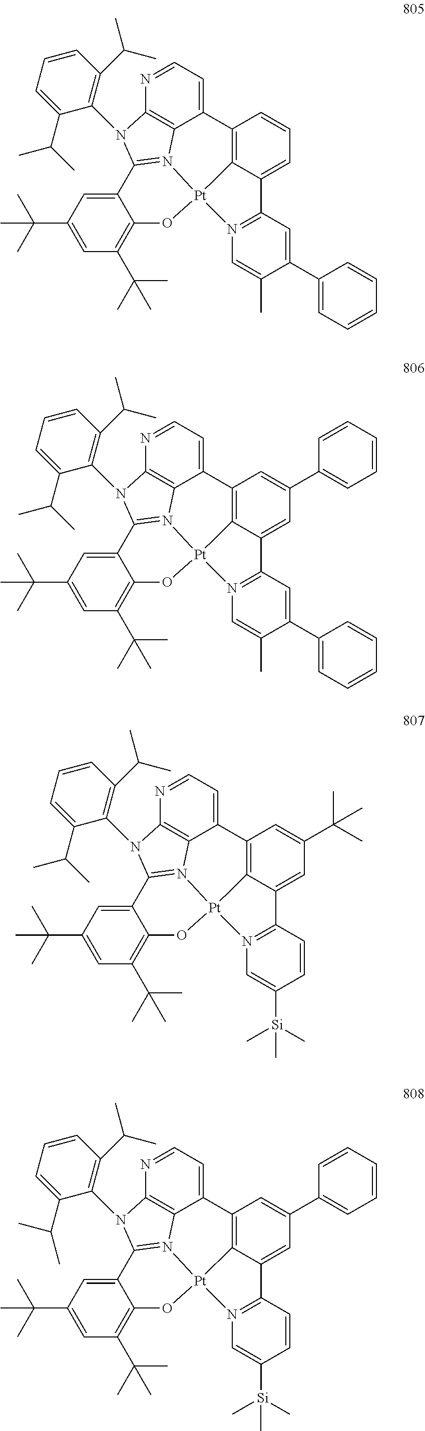

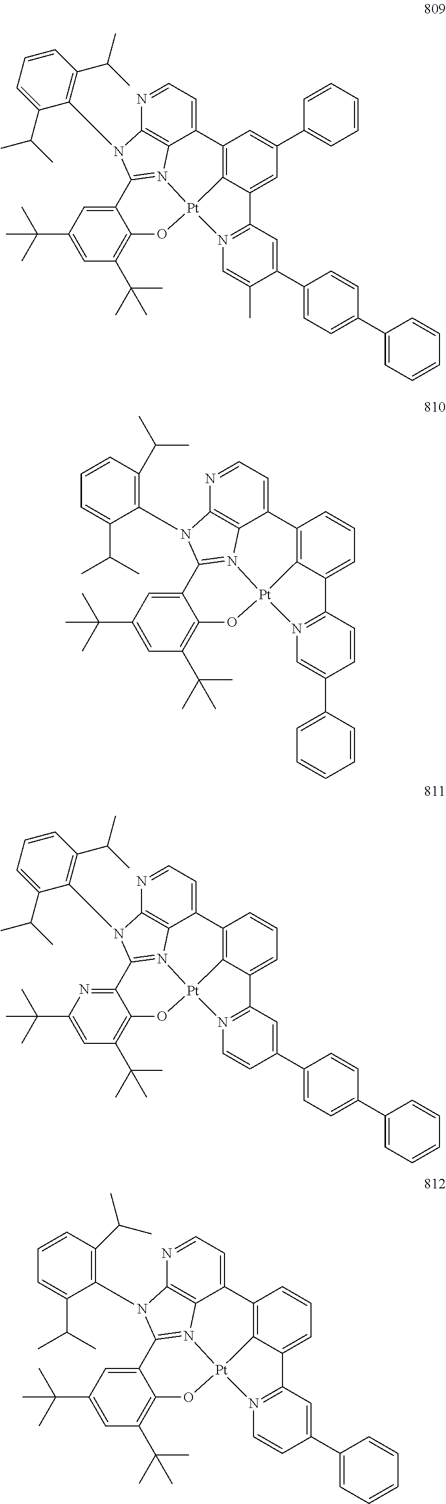

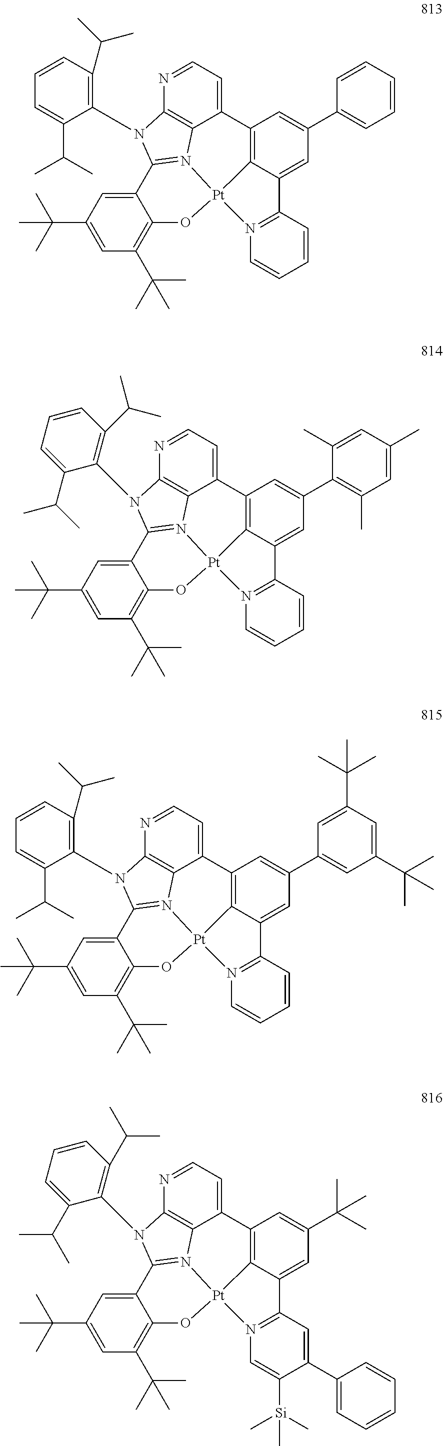

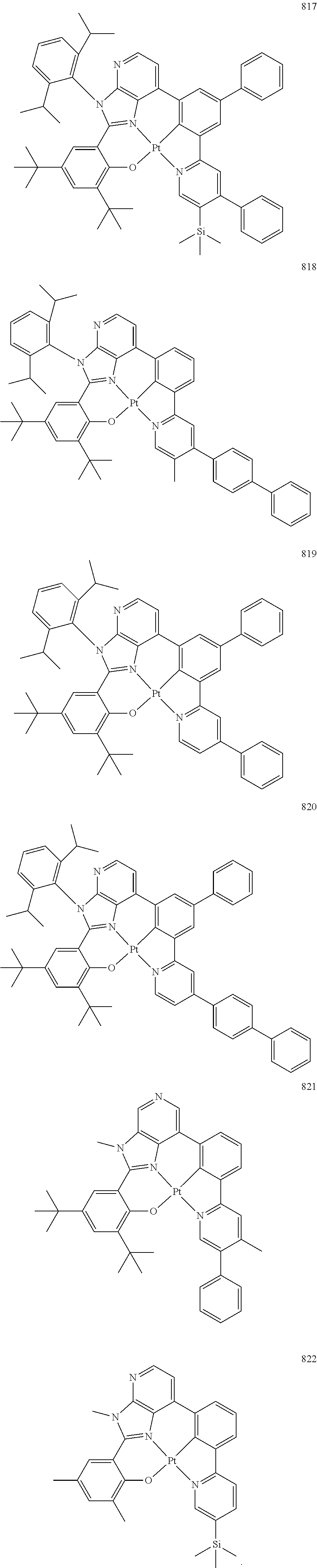

16. An organic light-emitting device comprising:
a first electrode;
a second electrode; and
an organic layer disposed between the first electrode and the second electrode,
wherein the organic layer comprises an emission layer and at least one organometallic compound of claim 1.

17. The organic light-emitting device of claim 16, wherein
the first electrode is an anode,
the second electrode is a cathode, and
the organic layer further comprises a hole transport region disposed between the first electrode and the emission layer and an electron transport region disposed between the emission layer and the second electrode,
wherein the hole transport region comprises a hole injection layer, a hole transport layer, an electron blocking layer, or any combination thereof, and
wherein the electron transport region comprises a hole blocking layer, an electron transport layer, an electron injection layer, or any combination thereof.

18. The electron transport region of claim 16, wherein
the emission layer comprises the organometallic compound.

19. The electron transport region of claim 18, wherein
the emission layer further comprises a host, wherein an amount of the host is larger than an amount of the organometallic compound.

20. A diagnostic composition comprising at least one organometallic compound of claim 1.
